(19) United States
(12) Patent Application Publication
(10) Pub. No.: US 2024/0335444 A1
(43) Pub. Date: Oct. 10, 2024

(54) MODULATORS OF BCL6 PROTEOLYSIS AND ASSOCIATED METHODS OF USE

(71) Applicant: Arvinas Operations, Inc., New Haven, CT (US)

(72) Inventors: Dan SHERMAN, Madison, CT (US); Sheryl Maxine GOUGH, Stratford, CT (US)

(21) Appl. No.: 18/407,150

(22) Filed: Jan. 8, 2024

Related U.S. Application Data
(60) Provisional application No. 63/437,994, filed on Jan. 9, 2023.
(60) Provisional application No. 63/618,208, filed on Jan. 5, 2024.

(51) Int. Cl. A61K 31/506; A61P 35/00

(52) U.S. Cl. CPC A61K 31/506; A61P 35/00

(57) ABSTRACT

This application pertains to the use of compounds, e.g., Compound A:

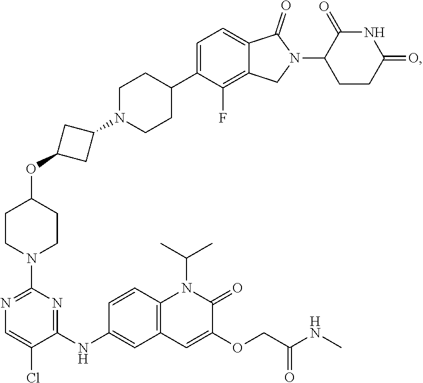 [SMILES: CNC(=O)COc1cc2cc(Nc3nc(N4CCC(O[C@H]5C[C@H](N6CCC(c7ccc8c(c7F)CN(C7CCC(=O)NC7=O)C8=O)CC6)C5)CC4)ncc3Cl)ccc2n(C(C)C)c1=O]

or a pharmaceutically acceptable salt thereof, for the treatment of various diseases or disorders, including, for example, diffuse large B-cell lymphoma (DLBCL) and angioimmunoblastic T-cell lymphoma.

### RELATED APPLICATIONS

This application claims priority to, and the benefit of, U.S. Provisional Application No. 63/437,994, filed Jan. 9, 2023, and U.S. Provisional Application No. 63/618,208, filed Jan. 5, 2024, which are incorporated by reference in their entireties for all purposes.

### FIELD OF THE INVENTION

The description provides compounds comprising a target protein binding moiety and a E3 ubiquitin ligase binding moiety, and associated methods of using those compounds. The compounds are useful as modulators of targeted ubiquitination, such as B-cell lymphoma 6 protein (BCL6), which is degraded by the compounds of the present disclosure.

### BACKGROUND

Most small molecule drugs bind enzymes or receptors in tight and well-defined pockets. On the other hand, protein-protein interactions are notoriously difficult to target using small molecules due to their large contact surfaces and the shallow grooves or flat interfaces involved. E3 ubiquitin ligases (of which hundreds are known in humans) confer substrate specificity for ubiquitination, and therefore, are more attractive therapeutic targets than general proteasome inhibitors due to their specificity for certain protein substrates. The development of ligands of E3 ligases has proven challenging, in part due to the fact that they must disrupt protein-protein interactions. However, recent developments have provided specific ligands which bind to these ligases. For example, since the discovery of nutlins, the first small molecule E3 ligase inhibitors, additional compounds have been reported that target E3 ligases but the field remains underdeveloped.

Cereblon is a protein that in humans is encoded by the CRBN gene. CRBN orthologs are highly conserved from plants to humans, which underscores its physiological importance. Cereblon forms an E3 ubiquitin ligase complex with damaged DNA binding protein 1 (DDB1), Cullin-4A (CUL4A), and regulator of cullins 1 (ROC1). This complex ubiquitinates a number of other proteins. Through a mechanism which has not been completely elucidated, cereblon ubiquitination of target proteins results in increased levels of fibroblast growth factor 8 (FGF8) and fibroblast growth factor 10 (FGF10). FGF8 in turn regulates a number of developmental processes, such as limb and auditory vesicle formation. The net result is that this ubiquitin ligase complex is important for limb outgrowth in embryos. In the absence of cereblon, DDB1 forms a complex with DDB2 that functions as a DNA damage-binding protein.

Bifunctional compounds such as those that are described in U.S. Patent Application Publications 2015-0291562 and 2014-0356322 (incorporated herein by reference), function to recruit endogenous proteins to an E3 ubiquitin ligase for degradation. In particular, the publications describe bifunctional or proteolysis targeting chimeric (PROTAC) compounds, which find utility as modulators of targeted ubiquitination of a variety of polypeptides and other proteins, which are then degraded and/or otherwise inhibited by the bifunctional compounds.

An ongoing need exists in the art for effective treatments for disease associated with (i) aberrant BCL6 expression and/or activity and/or (ii) overexpression or aggregation of B-cell lymphoma 6 protein (BCL6). However, non-specific effects, and the inability to target and modulate BCL6, remain as obstacles to the development of effective treatments. As such, small-molecule therapeutic agents that target BCL6 and that leverage or potentiate E3 ubiquitin ligase (e.g., cereblon's) substrate specificity would be very useful.

### SUMMARY

In one aspect, this application pertains to a method of treating or ameliorating diffuse large B-cell lymphoma (DLBCL), angioimmunoblastic T-cell lymphoma, transformed follicular lymphoma, high grade B-cell lymphoma, non-Hodgkin lymphoma not otherwise specified, or solid tumors in a subject in need thereof, comprising administering to the subject an effective amount of a compound, wherein the compound is:

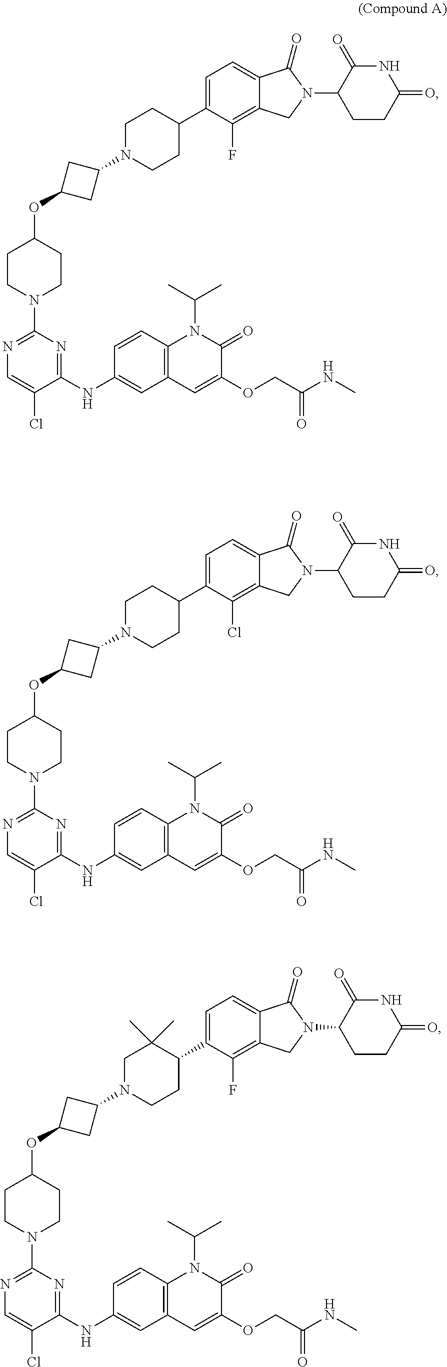 [SMILES: CNC(=O)COc1cc2cc(Nc3nc(N4CCC(O[C@H]5C[C@H](N6CCC(c7ccc8c(c7F)CN(C7CCC(=O)NC7=O)C8=O)CC6)C5)CC4)ncc3Cl)ccc2n(C(C)C)c1=O ; CNC(=O)COc1cc2cc(Nc3nc(N4CCC(O[C@H]5C[C@H](N6CCC(c7ccc8c(c7Cl)CN(C7CCC(=O)NC7=O)C8=O)CC6)C5)CC4)ncc3Cl)ccc2n(C(C)C)c1=O ; CNC(=O)COc1cc2cc(Nc3nc(N4CCC(O[C@H]5C[C@H](N6CC[C@@H](c7ccc8c(c7F)CN([C@H]7CCC(=O)NC7=O)C8=O)C(C)(C)C6)C5)CC4)ncc3Cl)ccc2n(C(C)C)c1=O]

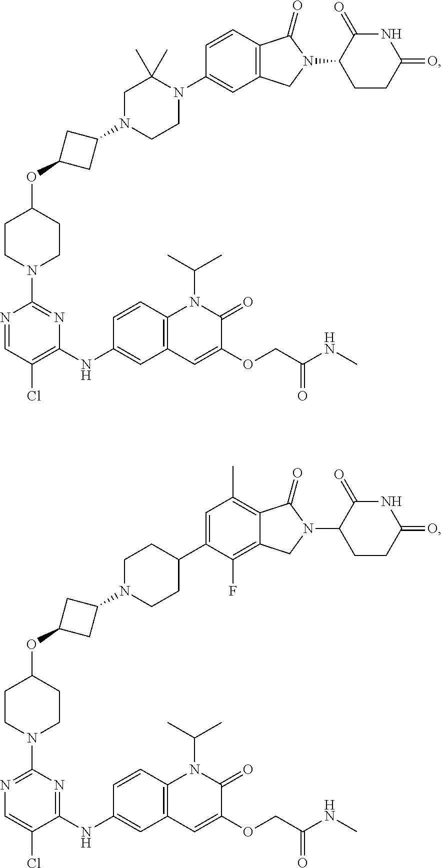 [SMILES: CNC(=O)COc1cc2cc(Nc3nc(N4CCC(O[C@H]5C[C@H](N6CCN(c7ccc8c(c7)CN([C@H]7CCC(=O)NC7=O)C8=O)C(C)(C)C6)C5)CC4)ncc3Cl)ccc2n(C(C)C)c1=O ; CNC(=O)COc1cc2cc(Nc3nc(N4CCC(O[C@H]5C[C@H](N6CCC(c7cc(C)c8c(c7F)CN(C7CCC(=O)NC7=O)C8=O)CC6)C5)CC4)ncc3Cl)ccc2n(C(C)C)c1=O]

or a pharmaceutically acceptable salt thereof.

In one aspect, this application pertains to a method of treating or ameliorating diffuse large B-cell lymphoma (DLBCL), angioimmunoblastic T-cell lymphoma, transformed follicular lymphoma, high grade B-cell lymphoma, non-Hodgkin lymphoma not otherwise specified, or solid tumors in a subject in need thereof, comprising administering to the subject an effective amount of a compound, wherein the compound is:

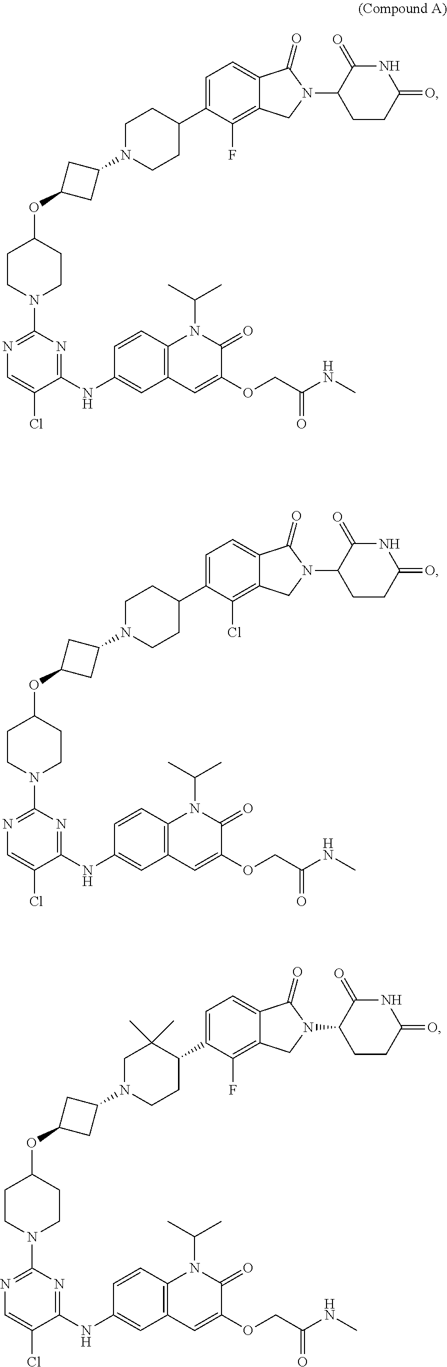 [SMILES: CNC(=O)COc1cc2cc(Nc3nc(N4CCC(O[C@H]5C[C@H](N6CCC(c7ccc8c(c7F)CN(C7CCC(=O)NC7=O)C8=O)CC6)C5)CC4)ncc3Cl)ccc2n(C(C)C)c1=O ; CNC(=O)COc1cc2cc(Nc3nc(N4CCC(O[C@H]5C[C@H](N6CCC(c7ccc8c(c7Cl)CN(C7CCC(=O)NC7=O)C8=O)CC6)C5)CC4)ncc3Cl)ccc2n(C(C)C)c1=O ; CNC(=O)COc1cc2cc(Nc3nc(N4CCC(O[C@H]5C[C@H](N6CC[C@@H](c7ccc8c(c7F)CN([C@H]7CCC(=O)NC7=O)C8=O)C(C)(C)C6)C5)CC4)ncc3Cl)ccc2n(C(C)C)c1=O]

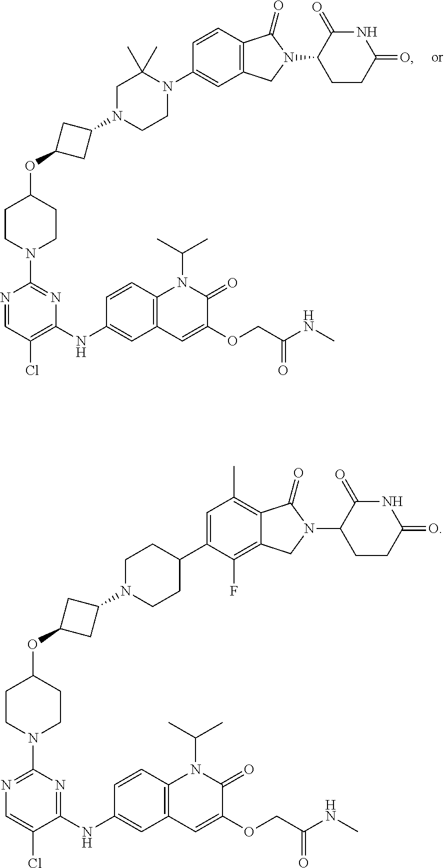 [SMILES: CNC(=O)COc1cc2cc(Nc3nc(N4CCC(O[C@H]5C[C@H](N6CCN(c7ccc8c(c7)CN([C@H]7CCC(=O)NC7=O)C8=O)C(C)(C)C6)C5)CC4)ncc3Cl)ccc2n(C(C)C)c1=O ; CNC(=O)COc1cc2cc(Nc3nc(N4CCC(O[C@H]5C[C@H](N6CCC(c7cc(C)c8c(c7F)CN(C7CCC(=O)NC7=O)C8=O)CC6)C5)CC4)ncc3Cl)ccc2n(C(C)C)c1=O]

In one aspect, this application pertains to a method of treating or ameliorating diffuse large B-cell lymphoma (DLBCL), angioimmunoblastic T-cell lymphoma, or solid tumors in a subject in need thereof, comprising administering to the subject an effective amount of a compound, wherein the compound is:

or a pharmaceutically acceptable salt thereof.

In one aspect, this application pertains to a method of treating or ameliorating diffuse large B-cell lymphoma (DLBCL), angioimmunoblastic T-cell lymphoma, or solid tumors in a subject in need thereof, comprising administering to the subject an effective amount of a compound, wherein the compound is:

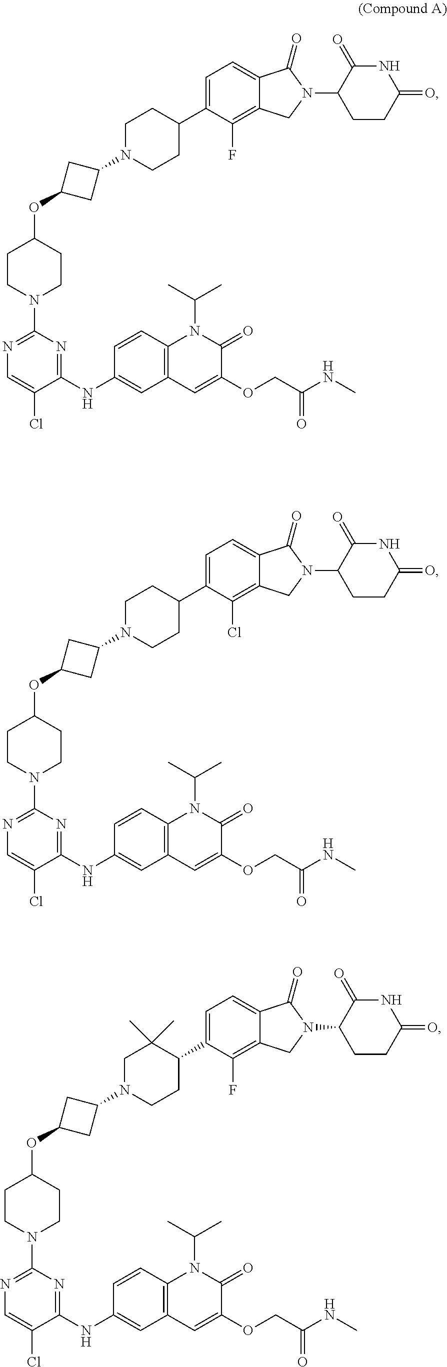 [SMILES: CNC(=O)COc1cc2cc(Nc3nc(N4CCC(O[C@H]5C[C@H](N6CCC(c7ccc8c(c7F)CN(C7CCC(=O)NC7=O)C8=O)CC6)C5)CC4)ncc3Cl)ccc2n(C(C)C)c1=O ; CNC(=O)COc1cc2cc(Nc3nc(N4CCC(O[C@H]5C[C@H](N6CCC(c7ccc8c(c7Cl)CN(C7CCC(=O)NC7=O)C8=O)CC6)C5)CC4)ncc3Cl)ccc2n(C(C)C)c1=O ; CNC(=O)COc1cc2cc(Nc3nc(N4CCC(O[C@H]5C[C@H](N6CC[C@@H](c7ccc8c(c7F)CN([C@H]7CCC(=O)NC7=O)C8=O)C(C)(C)C6)C5)CC4)ncc3Cl)ccc2n(C(C)C)c1=O]

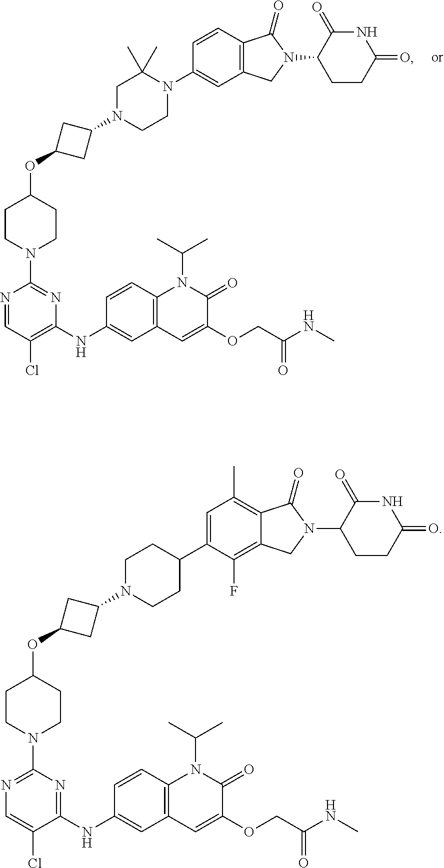 [SMILES: CNC(=O)COc1cc2cc(Nc3nc(N4CCC(O[C@H]5C[C@H](N6CCN(c7ccc8c(c7)CN([C@H]7CCC(=O)NC7=O)C8=O)C(C)(C)C6)C5)CC4)ncc3Cl)ccc2n(C(C)C)c1=O ; CNC(=O)COc1cc2cc(Nc3nc(N4CCC(O[C@H]5C[C@H](N6CCC(c7cc(C)c8c(c7F)CN(C7CCC(=O)NC7=O)C8=O)CC6)C5)CC4)ncc3Cl)ccc2n(C(C)C)c1=O]

In one aspect, this application pertains to a method of treating or ameliorating diffuse large B-cell lymphoma (DLBCL), angioimmunoblastic T-cell lymphoma, transformed follicular lymphoma, high grade B-cell lymphoma, non-Hodgkin lymphoma not otherwise specified, or solid tumors, comprising administering to the subject an effective amount of Compound A:

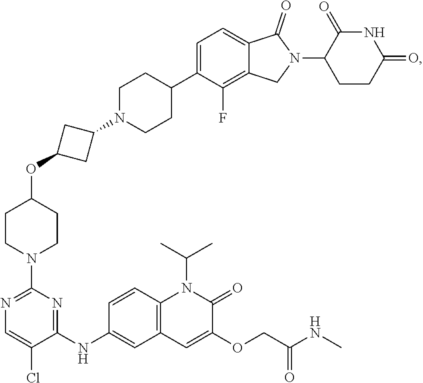 [SMILES: CNC(=O)COc1cc2cc(Nc3nc(N4CCC(O[C@H]5C[C@H](N6CCC(c7ccc8c(c7F)CN(C7CCC(=O)NC7=O)C8=O)CC6)C5)CC4)ncc3Cl)ccc2n(C(C)C)c1=O]

or a pharmaceutically acceptable salt thereof.

In one aspect, this application pertains to a method of treating or ameliorating diffuse large B-cell lymphoma (DLBCL), angioimmunoblastic T-cell lymphoma, transformed follicular lymphoma, high grade B-cell lymphoma, non-Hodgkin lymphoma not otherwise specified, or solid tumors, comprising administering to the subject an effective amount of Compound A:

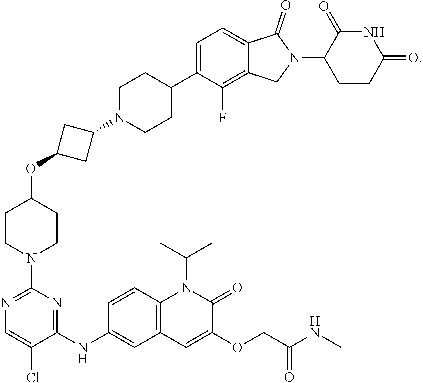 [SMILES: CNC(=O)COc1cc2cc(Nc3nc(N4CCC(O[C@H]5C[C@H](N6CCC(c7ccc8c(c7F)CN(C7CCC(=O)NC7=O)C8=O)CC6)C5)CC4)ncc3Cl)ccc2n(C(C)C)c1=O]

In one aspect, this application pertains to a method of treating or ameliorating diffuse large B-cell lymphoma (DLBCL), comprising administering to the subject an effective amount of Compound A:

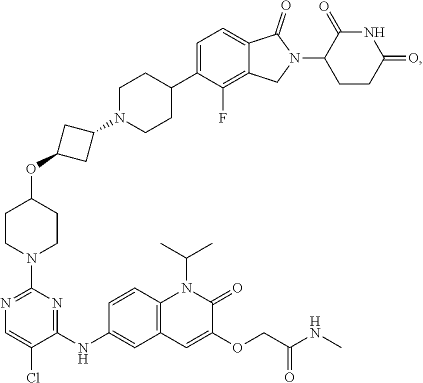 [SMILES: CNC(=O)COc1cc2cc(Nc3nc(N4CCC(O[C@H]5C[C@H](N6CCC(c7ccc8c(c7F)CN(C7CCC(=O)NC7=O)C8=O)CC6)C5)CC4)ncc3Cl)ccc2n(C(C)C)c1=O]

or a pharmaceutically acceptable salt thereof.

In one aspect, this application pertains to a method of treating or ameliorating diffuse large B-cell lymphoma (DLBCL), comprising administering to the subject an effective amount of Compound A:

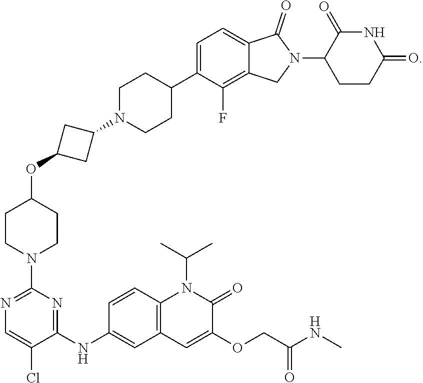 [SMILES: CNC(=O)COc1cc2cc(Nc3nc(N4CCC(O[C@H]5C[C@H](N6CCC(c7ccc8c(c7F)CN(C7CCC(=O)NC7=O)C8=O)CC6)C5)CC4)ncc3Cl)ccc2n(C(C)C)c1=O]

In one aspect, this application pertains to a method of treating or ameliorating angioimmunoblastic T-cell lymphoma in a subject in need thereof, comprising administering to the subject an effective amount of Compound A:

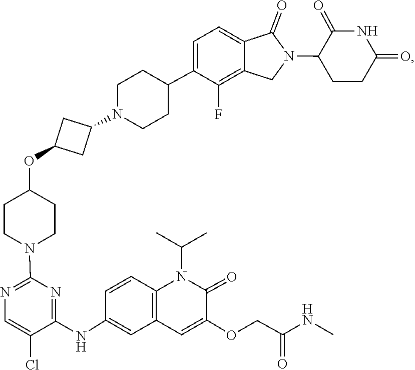 [SMILES: CNC(=O)COc1cc2cc(Nc3nc(N4CCC(O[C@H]5C[C@H](N6CCC(c7ccc8c(c7F)CN(C7CCC(=O)NC7=O)C8=O)CC6)C5)CC4)ncc3Cl)ccc2n(C(C)C)c1=O]

or a pharmaceutically acceptable salt thereof.

In one aspect, this application pertains to a method of treating or ameliorating angioimmunoblastic T-cell lymphoma in a subject in need thereof, comprising administering to the subject an effective amount of Compound A:

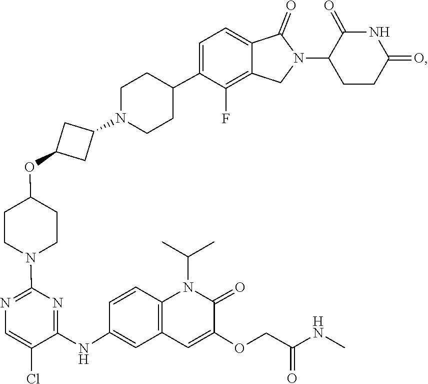 [SMILES: CNC(=O)COc1cc2cc(Nc3nc(N4CCC(O[C@H]5C[C@H](N6CCC(c7ccc8c(c7F)CN(C7CCC(=O)NC7=O)C8=O)CC6)C5)CC4)ncc3Cl)ccc2n(C(C)C)c1=O]

In one aspect, this application pertains to a method of treating solid tumors in a subject in need thereof, comprising administering to the subject an effective amount of Compound A:

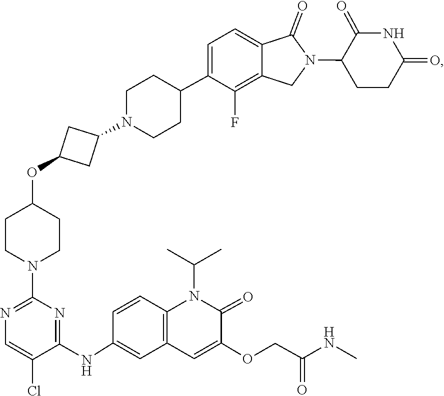 [SMILES: CNC(=O)COc1cc2cc(Nc3nc(N4CCC(O[C@H]5C[C@H](N6CCC(c7ccc8c(c7F)CN(C7CCC(=O)NC7=O)C8=O)CC6)C5)CC4)ncc3Cl)ccc2n(C(C)C)c1=O]

or a pharmaceutically acceptable salt thereof.

In one aspect, this application pertains to a method of treating solid tumors in a subject in need thereof, comprising administering to the subject an effective amount of Compound A:

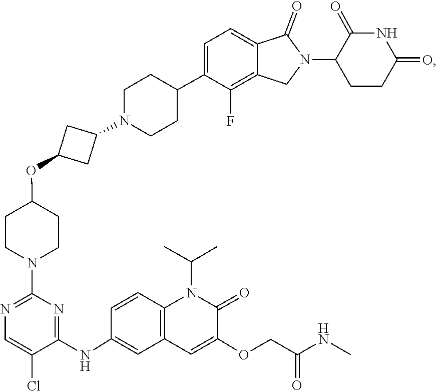 [SMILES: CNC(=O)COc1cc2cc(Nc3nc(N4CCC(O[C@H]5C[C@H](N6CCC(c7ccc8c(c7F)CN(C7CCC(=O)NC7=O)C8=O)CC6)C5)CC4)ncc3Cl)ccc2n(C(C)C)c1=O]

### BRIEF DESCRIPTION OF THE DRAWINGS

The accompanying drawings, which are incorporated into and form a part of the specification, illustrate several embodiments of the present disclosure and, together with the description, serve to explain the principles of the disclosure. The drawings are only for the purpose of illustrating an embodiment of the disclosure and are not to be construed as limiting the disclosure. Further objects, features and advantages of the disclosure will become apparent from the following detailed description taken in conjunction with the accompanying figures showing illustrative embodiments of the disclosure, in which:

FIG. 1A-1B. Illustration of general principle for the heterobifunctional degradative Compound A of the present disclosure. FIG. 1A Exemplary heterobifunctional degradative Compound A comprises a protein targeting moiety (PTM; rectangle), a ubiquitin ligase binding moiety (ULM; triangle), and optionally a linker moiety (L; bold black line) coupling or tethering the PTM to the ULM. FIG. 1B Illustrates the functional use of the heterobifunctional degradative Compound A as described herein. Briefly, the ULM recognizes and binds to a specific E3 ubiquitin ligase, and the PTM binds and recruits a target protein bringing it into close proximity to the E3 ubiquitin ligase. Typically, the E3 ubiquitin ligase is complexed with an E2 ubiquitin-conjugating protein, and either alone or via the E2 protein catalyzes attachment of ubiquitin (small filled ovals) to a lysine on the target protein via an isopeptide bond. The poly-ubiquitinated protein (far right) is then targeted for degradation by the proteasomal machinery of the cell.

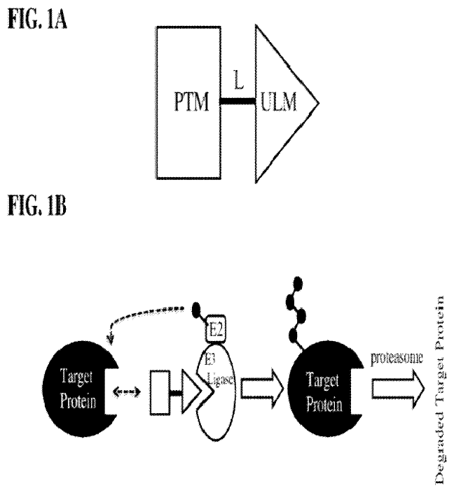

FIG. 2A-2C. Compound A degradation of BCL6 protein in germinal center B-cell (GCB) and activated B-cell (ABC) DLBCL cell lines. FIG. 2A OCI-Ly1 was treated with Compound A and its E3-binding deficient analogue for 24-hours. FIG. 2B GCB DLBCL cell lines Farage, SU-DHL-4, SU-DHL-6 and OCI-Ly7 and FIG. 2C ABC DLBCL lines SU-DHL-2 and OCI-Ly10, were treated for 24 hours with Compound A.

FIG. 3A-3B. Antiproliferative effects of Compound A on DLBCL-derived cell lines in 9-day cell growth inhibition assays. FIG. 3A GCB lines OCI-Ly1, OCI-Ly7, SU-DHL-4, and SU-DHL-6, and FIG. 3B ABC lines SU-DHL-2 and OCI-Ly10 were dosed with a 7-point 3-fold serial dilution of Compound A at a top dose of 30 nM.

FIG. 4A Average tumor growth of DLBCL cell line derived xenograft model OCI-Ly1 with a treatment of Compound A. FIG. 4B Average body weights of treated mice.

FIG. 5. Tumor lysate BCL6 protein levels in OCI-Ly1 cell line xenograft tumor tissue following a treatment time-course with Compound A.

FIG. 6. Total Compound A plasma and tumor levels (related to FIG. 5).

FIG. 7A Average tumor growth of DLBCL cell line derived xenograft model OCI-Ly1 with a treatment of Compound A. FIG. 7B Average body weights of treated mice. FIG. 7C Tumor lysate BCL6 protein levels 16-hours post-last dose analyzed by western blotting.

FIG. 8A Average tumor growth of DLBCL cell line derived xenograft model OCI-Ly7 with a treatment of Compound A. FIG. 8B Average body weights of treated mice. FIG. 8C Tumor lysate BCL6 protein levels 16-hours post-last dose analyzed by western blotting.

FIG. 9A Average tumor growth of DLBCL cell line derived xenograft model SU-DHL-2 with a treatment of Compound A. FIG. 9B Average body weights of treated mice. FIG. 9C Tumor lysate BCL6 protein levels 16-hours post-last dose analyzed by western blotting.

FIG. 10A Average tumor growth of DLBCL cell line derived xenograft model OCI-Ly10 with a treatment of Compound A. FIG. 10B Average body weights of treated mice. FIG. 10C Tumor lysate BCL6 protein levels 16-hours post-last dose analyzed by western blotting.

FIGS. 11A-11D shows change in mean tumor volume for patient-derived xenograft (PDX) mouse models of diffuse large B-cell lymphoma (DLBCL), Burkitt's lymphoma, and non-Hodgkin lymphoma (NHL) not otherwise specified (NOS) treated with Compound A or vehicle as described in Example 10. Data points represent the mean per group and error bars the standard error of the mean.

### DETAILED DESCRIPTION

The following is a detailed description provided to aid those skilled in the art in practicing the present disclosure. Those of ordinary skill in the art may make modifications and variations in the embodiments described herein without departing from the spirit or scope of the present disclosure. All publications, patent applications, patents, figures and other references mentioned herein are expressly incorporated by reference in their entirety.

Unless otherwise defined, all technical and scientific terms used herein have the same meaning as commonly understood by one of ordinary skill in the art to which this disclosure belongs. The terminology used in the description is for describing particular embodiments only, and is not intended to be limiting of the disclosure.

Where a range of values is provided, it is understood that each intervening value, to the tenth of the unit of the lower limit unless the context clearly dictates otherwise (such as in the case of a group containing a number of carbon atoms in which case each carbon atom number falling within the range is provided), between the upper and lower limit of that range and any other stated or intervening value in that stated range is encompassed within the disclosure. The upper and lower limits of these smaller ranges may independently be included in the smaller ranges is also encompassed within the disclosure, subject to any specifically excluded limit in the stated range. Where the stated range includes one or both of the limits, ranges excluding either or both of those included limits are also included in the disclosure.

The following terms are used to describe the present disclosure. In instances where a term is not specifically defined herein, that term is given an art-recognized meaning by those of ordinary skill applying that term in context to its use in describing the present disclosure.

The articles “a” and “an” as used herein and in the appended claims are used herein to refer to one or to more than one (i.e., to at least one) of the grammatical object of the article unless the context clearly indicates otherwise. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

It should also be understood that, in certain methods described herein that include more than one step or act, the order of the steps or acts of the method is not necessarily limited to the order in which the steps or acts of the method are recited unless the context indicates otherwise.

The term “ubiquitin ligase” refers to a family of proteins that facilitate the transfer of ubiquitin to a specific substrate protein, targeting the substrate protein for degradation. For example, cereblon an E3 ubiquitin ligase protein that alone or in combination with an E2 ubiquitin-conjugating enzyme causes the attachment of ubiquitin to a lysine on a target protein, and subsequently targets the specific protein substrates for degradation by the proteasome. Thus, E3 ubiquitin ligase alone or in complex with an E2 ubiquitin conjugating enzyme is responsible for the transfer of ubiquitin to targeted proteins. In general, the ubiquitin ligase is involved in polyubiquitination such that a second ubiquitin is attached to the first; a third is attached to the second, and so forth. Polyubiquitination marks proteins for degradation by the proteasome. However, there are some ubiquitination events that are limited to mono-ubiquitination, in which only a single ubiquitin is added by the ubiquitin ligase to a substrate molecule. Mono-ubiquitinated proteins are not targeted to the proteasome for degradation, but may instead be altered in their cellular location or function, for example, via binding other proteins that have domains capable of binding ubiquitin. Further complicating matters, different lysines on ubiquitin can be targeted by an E3 to make chains. The most common lysine is Lys48 on the ubiquitin chain. This is the lysine used to make polyubiquitin, which is recognized by the proteasome.

The term “patient” or “subject” is used throughout the specification to describe an animal, preferably a human or a domesticated animal, to whom treatment, including prophylactic treatment, with the compositions according to the present disclosure is provided. For treatment of those infections, conditions or disease states which are specific for a specific animal such as a human patient, the term patient refers to that specific animal, including a domesticated animal such as a dog or cat or a farm animal such as a horse, cow, sheep, etc. In general, in the present disclosure, the term patient refers to a human patient unless otherwise stated or implied from the context of the use of the term.

The term “effective” is used to describe an amount of a compound, composition or component which, when used within the context of its intended use, effects an intended result. The term effective subsumes all other effective amount or effective concentration terms, which are otherwise described or used in the present application.

### Compounds of the Disclosure

In some embodiments, the compound of the disclosure has the following chemical structure:

CLM-L-PTM

or a pharmaceutically acceptable salt thereof,
wherein:
(a) the CLM is a cereblon E3 ubiquitin ligase binding moiety represented by:

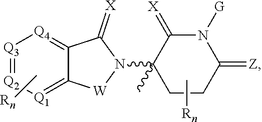

wherein:
   W is CH₂, O, CHR (e.g., CH(CH₃)), C═O, NH, or N;
   each X is independently selected from absent, O, S, and CH₂;
   Z is O, S, or CH₂;
   G is H, methyl, or OH;
   each of Q₁, Q₂, Q₃, and Q₄ independently represent a N or a C substituted with an H or an R;
   A is H, unsubstituted or substituted linear or branched alkyl, Cl, or F;
   n is an integer from 1 to 4 (e.g., 1 or 2, 1-3, 1, 2, 3, or 4);
   each R is independently bond, H, —OR′, —NR′R″, —CR′R″—, -unsubstituted or substituted linear or branched C1-C6 linear or branched alkyl (e.g. C1-C3 alkyl and/or optionally substituted with one or more halogen), unsubstituted or substituted alkoxyl group (e.g., a methoxy, ethoxy, butoxy, propoxy, pentoxy, or hexoxy; wherein the alkoxyl optionally substituted with one or more halogen, C1-C3 alkyl, haloalkyl, or C1-C3 fluoroalkyl), optionally substituted 4-6 membered cycloalkyl, optionally substituted 4-6 membered heterocycloalkyl, —Cl, —F, —Br, —I, —CF₃, —CN, or —NO₂, wherein one R is covalently joined to the L;
   R′ and R″ are each independently selected from bond H, and substituted or unsubstituted C1-C4 alkyl (e.g., methyl or ethyl); and
   [symbol] represents a bond that may be stereospecific ((R) or (S)) or non-stereospecific;
(b) the PTM is a small molecule comprising a B-cell lymphoma 6 protein (BCL6) targeting moiety selected from:

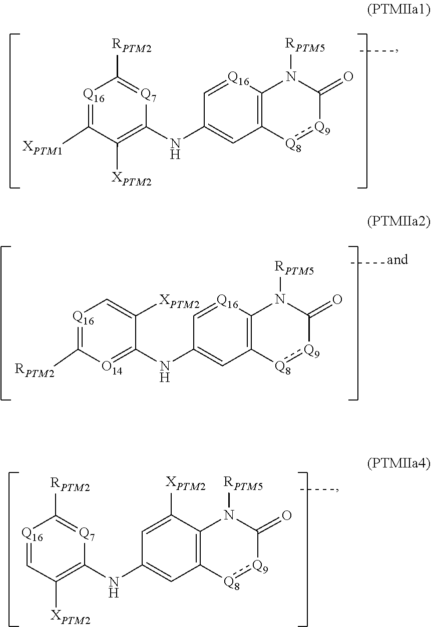

wherein:
   R_{PTM5} is H, optionally substituted linear or branched C1-C6 alkyl (e.g., methyl, ethyl, or isopropyl group), C1-C4 alkyl-O(C1-C3 alkyl), C1-C4 alkyl-O—, C1-C4 alkyl-NH(C1-C3 alkyl), C1-C4 alkyl-N(C1-C3 alkyl)₂, optionally substituted C5-C10 aryl, optionally substituted C5-C10 heteroaryl, optionally substituted C3-C10 cycloalkyl, or optionally substituted C3-C10 heterocyclyl;
   Q₆ and Q₁₆ are each independently N or CH;
   Q₇ and Q₁₄ are each independently N or CH;
   X_{PTM1} is H, Cl, or F;
   X_{PTM2} is H, Cl, F, or CN;
   [symbol] of Q₈ and Q₉ is a single bond or a double bond, wherein
      when Q₈ and Q₉ are connected by a single bond:
         Q₈ is CH₂; and
         Q₉ is CH(R_{PTM3}), or N(R_{PTM3});
      when Q₈ and Q₉ are connected by a double bond:
         Q₈ is CH; and
         Q₉ is C(R_{PTM3});
   R_{PTM3} is: —OH; —Cl; —F; —CN; optionally substituted linear or branched C1-C6 alkyl, optionally substituted C1-C6 alkoxy (e.g., —OCH₃, or —OCH₂CH₃); optionally substituted

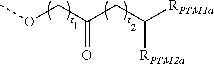

(e.g., optionally substituted with a linear or branched C1-C4 alkyl, C1-C4 alkoxy, —Cl; —F, —CN, or —OH); or optionally substituted

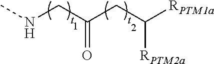

(e.g., optionally substituted with a linear or branched C1-C4 alkyl, C1-C4 alkoxy, —Cl, —F, —CN, or —OH);
each R_{PTM1}a and R_{PTM2}a is independently H, optionally substituted C1-C4 alkyl (e.g., a CH₃ or CH₂CH₃), optionally substituted C1-C4 alkoxy (e.g., —OCH₃ or —OCH₂CH₃), or CH₂OCH₃;
each t₁ is independently 1, 2, 3, 4, or 5; and
each t₂ is independently 0, 1, 2, 3, 4, or 5;
R_{PTM2} is H, OH, CN, —F, —Cl, optionally substituted linear or branched C1-C4 alkyl, optionally substituted —NH₂ (e.g., —N(C1-C3 alkyl)₂ or —NH(C1-C3 alkyl)), optionally substituted linear or branched —O—C1-C4 alkyl, an optionally substituted monocyclic or bicyclic C3-C12 heterocycloalkyl (e.g., azetidinel-yl, azetidinel-yl-3-ol, pyrrolidin-1-yl, piperidin-lyl, piperazin-1-yl, or morpholin-4-yl, homopiperazin-1-yl,

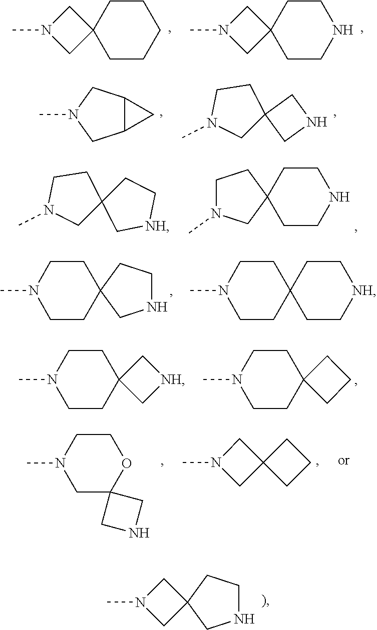

each optionally substituted with one or more of OH, a linear or branched C1-C6 alkyl, C1-C6 alkoxy —CN, —F, —Cl, or NH₂), an optionally substituted —O—C₃₋₁₂ monocyclic or bicyclic heterocycloalkyl (e.g. optionally substituted with one or more OH, a linear or branched C1-C6 alkyl, C1-C6 alkoxy, —CN, —F, —Cl, or NH₂), or an optionally substituted C3-C12 cycloalkyl (e.g., optionally substituted with one or more of OH, linear or branched C1-C6 alkyl, C1-C6 alkoxy, —CN, —F, —Cl, or NH₂), an optionally substituted C5-C6 heteroaryl (e.g. optionally substituted with one or more linear or branched C1-C6 alkyl, C1-C6 alkoxy, —CN, —F, —Cl, or NH₂), or an optionally substituted C5-C6 aryl (e.g. optionally substituted with one or more linear or branched C1-C6 alkyl, C1-C6 alkoxy, —CN, —F, —Cl, or NH₂); and
the [symbol] of the PTM indicates the point of attachment with the L; and
(c) the L is a chemical linker group covalently connecting the CLM and the PTM, that is represented by the formula:

-(A^{L})_{q}-,

wherein:
-(A^{L})_{q}- is a group which is connected to the CLM and the PTM;
q is an integer greater than or equal to 1;
each A is independently selected from CR^{L1}R^{L2}, O, S, SO, SO₂, NR^{L3} SO₂NR^{L3}, SONR^{L3}, CONR^{L3}, NR^{L3}CONR^{L4} NR^{L3}SO₂NR^{L4}, CO, CR^{L1}=CR^{L2}, C≡C, C₃₋₁₁ monocyclic or bicyclic cycloalkyl optionally substituted with 1-6 R^{L1} and/or R^{L2} groups, C₅₋₁₃ spirocycloalkyl optionally substituted with 1-9 R^{L1} and/or R^{L2} groups, C₃₋₁₁ monocyclic or bicyclic heterocyclyl optionally substituted with 1-6 R^{L1} and/or R^{L2} groups, C5-13 spiroheterocyclyl optionally substituted with 1-8 R^{L1} and/or R^{L2} groups, aryl optionally substituted with 1-6 R^{L1} and/or R^{L2} groups, and heteroaryl optionally substituted with 1-6 R^{L1} and/or R^{L2} groups; and
each R^{L1}, R^{L2}, R^{L3}, R^{L4} and R^{L5} is independently H, halogen, C₁₋₈alkyl, OC₁₋₈alkyl, SC₁₋₈alkyl, NHC₁₋₈alkyl, N(C₁₋₈alkyl)₂, C₃₋₁₁cycloalkyl, 5- or 6-membered aryl, 5- or 6-membered heteroaryl, C₃₋₁₁heterocyclyl, OC₃₋₈cycloalkyl, SC₃₋₈cycloalkyl, NHC₃₋₈cycloalkyl, N(C₃₋₈cycloalkyl)₂, N(C₃₋₈cycloalkyl)(C₁₋₈alkyl), OH, NH₂, SH, SO₂C₁₋₈alkyl, CC—C₁₋₈alkyl, CCH, CH═CH(C₁₋₈alkyl), C(C₁₋₈alkyl)═CH(C₁₋₈alkyl), C(C₁₋₈alkyl)═C(C₁₋₈alkyl)₂, COC₁₋₈alkyl, CO₂H, CN, CF₃, CHF₂, CH₂F, NO₂, SF₅, SO₂NHC₁₋₈alkyl, SO₂N(C₁₋₈alkyl)₂, SONHC₁₋₈alkyl, SON(C₁₋₈alkyl)₂, CONHC₁₋₈alkyl, CON(C₁₋₈alkyl)₂, N(C₁₋₈alkyl)CONH(C₁₋₈alkyl), N(C₁₋₈alkyl)CON(C₁₋₈alkyl)₂, NHCONH(C₁₋₈alkyl), NHCON(C₁₋₈alkyl)₂, NHCONH₂, N(C₁₋₈alkyl)SO₂NH(C₁₋₈alkyl), N(C₁₋₈alkyl) SO₂N(C₁₋₈alkyl)₂, NHSO₂NH(C₁₋₈alkyl), NHSO₂N(C₁₋₈alkyl)₂, or NHSO₂NH₂.

In some embodiments, the compound of the disclosure has a PTM that is selected from:

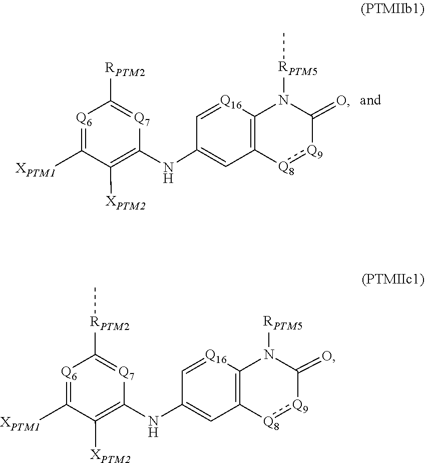

wherein the [symbol] of the PTM indicates the point of attachment with the L.

In some embodiments, the compound of the disclosure has a PTM that is selected from:

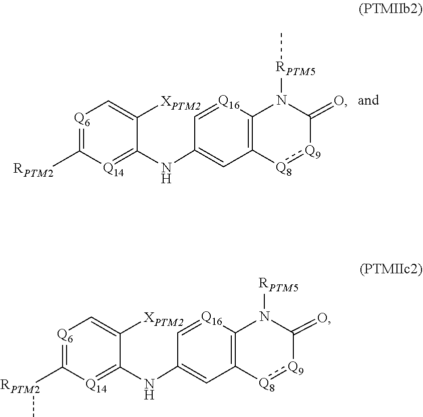

wherein the [symbol] of the PTM indicates the point of attachment with the L.

In some embodiments, the compound of the disclosure has a PTM that is selected from:

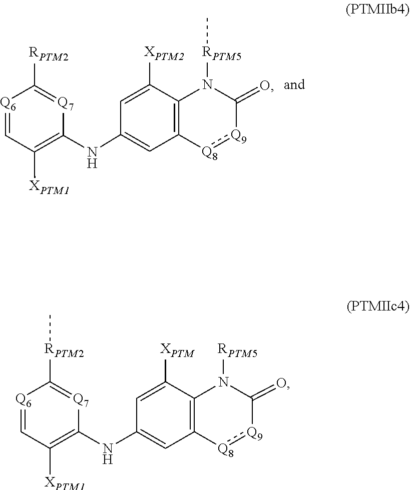

wherein the [symbol] of the PTM indicates the point of attachment with the L.

In some embodiments, the compound of the disclosure has at least one of:
(a) R_{PTM2} of PTMIIa1, PTMIIa2, PTMIIa4, PTMIIb1, PTMIIb2, PTMIIb4, PTMIIc1, PTMIIc2, and PTMIIc4 is selected from: H, OH, NH₂, —N(CH₃)₂, methyl, ethyl

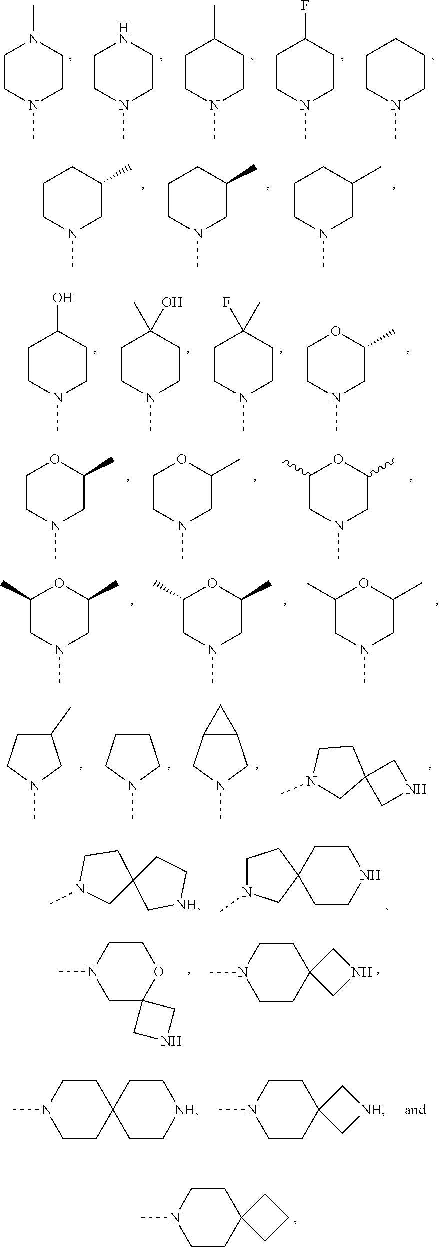

wherein [symbol] represents a bond that may be stereospecific ((R) or (S)) or non-stereospecific and [symbol] indicates the point of attachment to the aryl or heteroaryl of the PTM;
(c) R_{PTM3} of PTMIIa1, PTMIIa2, PTMIIa4, PTMIIb1, PTMIIb2, PTMIIb4, PTMIIc1, PTMIIc2, and PTMIIc4 is selected from:

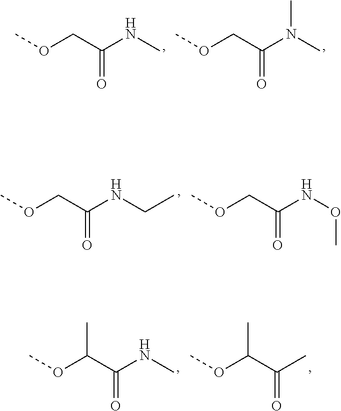

wherein [symbol] indicates the point of attachment of R_{PTM3} to the biheteroaryl or biheterocycle of the PTM; or
(d) R_{PTM5} of PTMIIa1, PTMIIa2, PTMIIa4, PTMIIb1, PTMIIb2, PTMIIb4, PTMIIc1, PTMIIc2, and PTMIIc4 is selected from: H, methyl, CFH₂, CF₂H, ethyl, propyl, isopropyl, cyclopropyl, butyl, pentyl, hexyl,

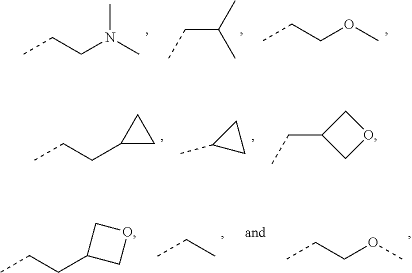

wherein [symbol] indicates the point of attachment of R_{PTM5} to the nitrogen of the biheteroaryl or biheterocycle of the PTM; or
(e) any combination of (a), (b), (c), and (d).

In some embodiments, the compound of the disclosure has a PTM that is:

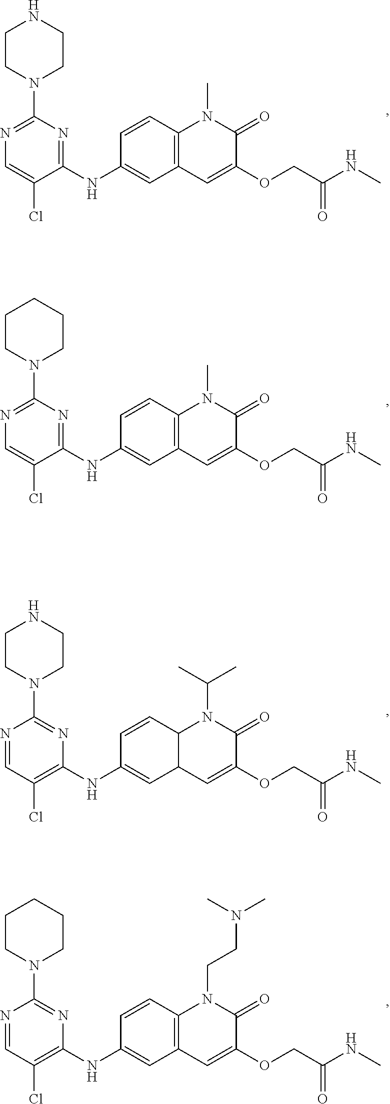 [SMILES: CNC(=O)COc1cc2cc(Nc3nc(N4CCNCC4)ncc3Cl)ccc2n(C)c1=O ; CNC(=O)COc1cc2cc(Nc3nc(N4CCCCC4)ncc3Cl)ccc2n(C)c1=O ; CNC(=O)COC1=CC2C=C(Nc3nc(N4CCNCC4)ncc3Cl)C=CC2N(C(C)C)C1=O ; CNC(=O)COc1cc2cc(Nc3nc(N4CCCCC4)ncc3Cl)ccc2n(CCN(C)C)c1=O]

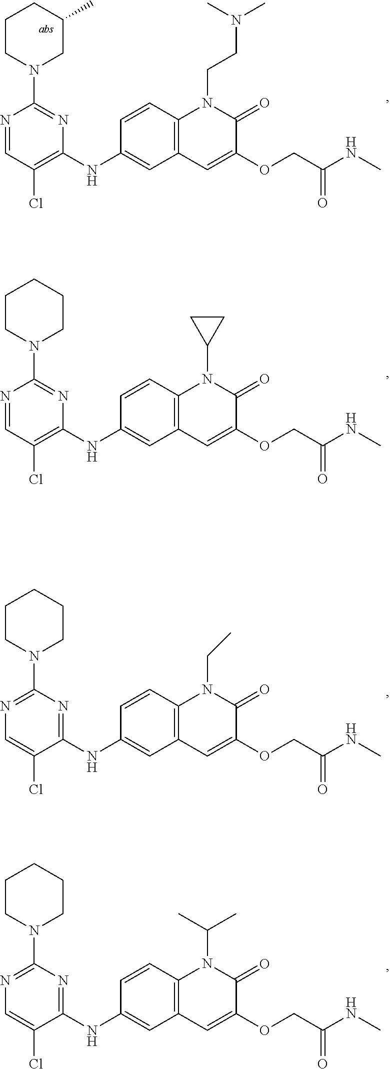 [SMILES: CNC(=O)COc1cc2cc(Nc3nc(N4CCC[C@H](C)C4)ncc3Cl)ccc2n(CCN(C)C)c1=O ; CNC(=O)COc1cc2cc(Nc3nc(N4CCCCC4)ncc3Cl)ccc2n(C2CC2)c1=O ; CCn1c(=O)c(OCC(=O)NC)cc2cc(Nc3nc(N4CCCCC4)ncc3Cl)ccc21 ; CNC(=O)COc1cc2cc(Nc3nc(N4CCCCC4)ncc3Cl)ccc2n(C(C)C)c1=O]

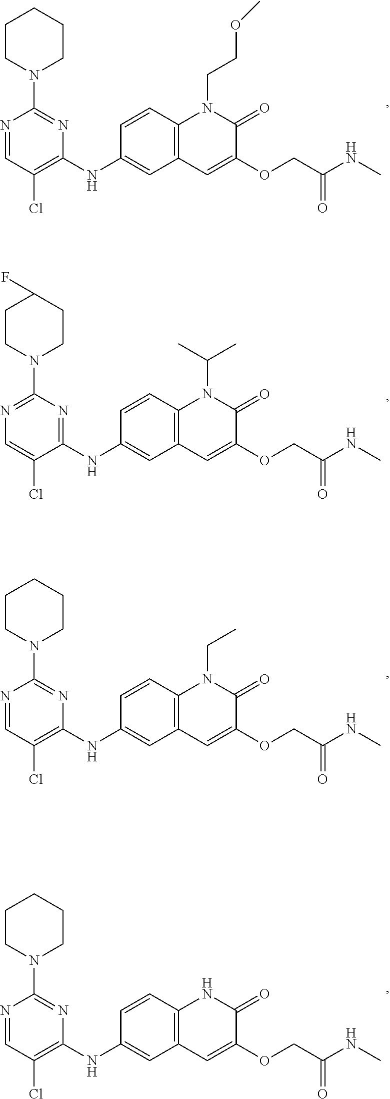 [SMILES: CNC(=O)COc1cc2cc(Nc3nc(N4CCCCC4)ncc3Cl)ccc2n(CCOC)c1=O ; CNC(=O)COc1cc2cc(Nc3nc(N4CCC(F)CC4)ncc3Cl)ccc2n(C(C)C)c1=O ; CCn1c(=O)c(OCC(=O)NC)cc2cc(Nc3nc(N4CCCCC4)ncc3Cl)ccc21 ; CNC(=O)COc1cc2cc(Nc3nc(N4CCCCC4)ncc3Cl)ccc2[nH]c1=O]

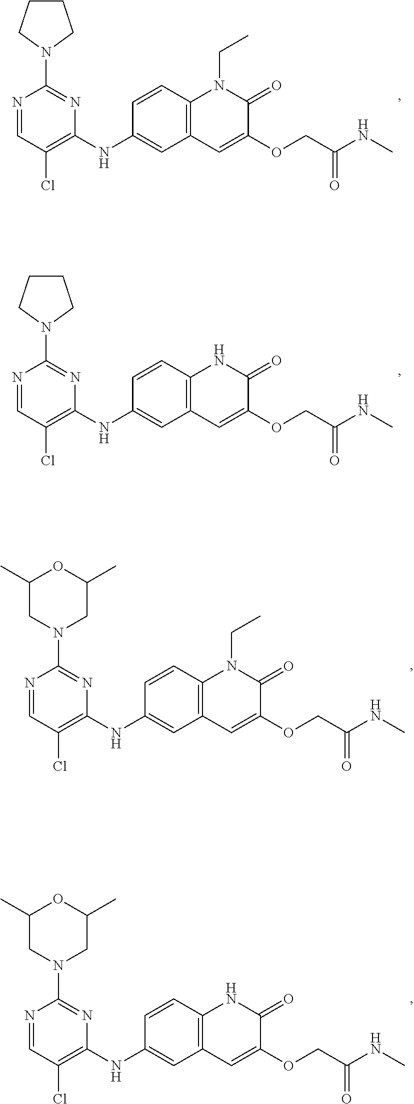

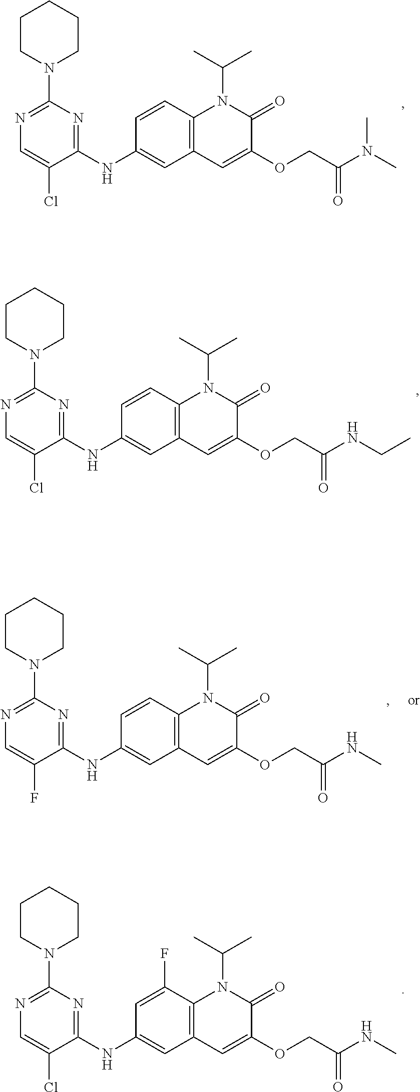 [SMILES: CC(C)n1c(=O)c(OCC(=O)N(C)C)cc2cc(Nc3nc(N4CCCCC4)ncc3Cl)ccc21 ; CCNC(=O)COc1cc2cc(Nc3nc(N4CCCCC4)ncc3Cl)ccc2n(C(C)C)c1=O ; CNC(=O)COc1cc2cc(Nc3nc(N4CCCCC4)ncc3F)ccc2n(C(C)C)c1=O ; CNC(=O)COc1cc2cc(Nc3nc(N4CCCCC4)ncc3Cl)cc(F)c2n(C(C)C)c1=O]

In some embodiments, the compound of the disclosure has a PTM that is:

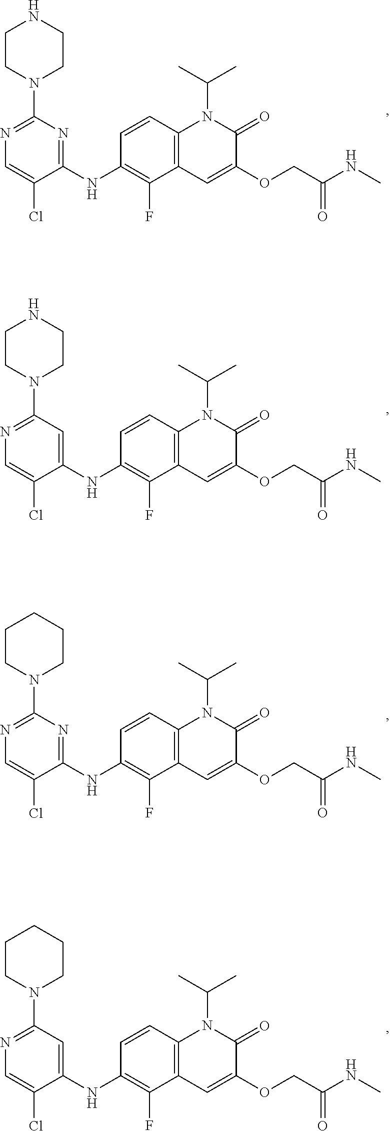 [SMILES: CNC(=O)COc1cc2c(F)c(Nc3nc(N4CCNCC4)ncc3Cl)ccc2n(C(C)C)c1=O ; CNC(=O)COc1cc2c(F)c(Nc3cc(N4CCNCC4)ncc3Cl)ccc2n(C(C)C)c1=O ; CNC(=O)COc1cc2c(F)c(Nc3nc(N4CCCCC4)ncc3Cl)ccc2n(C(C)C)c1=O ; CNC(=O)COc1cc2c(F)c(Nc3cc(N4CCCCC4)ncc3Cl)ccc2n(C(C)C)c1=O]

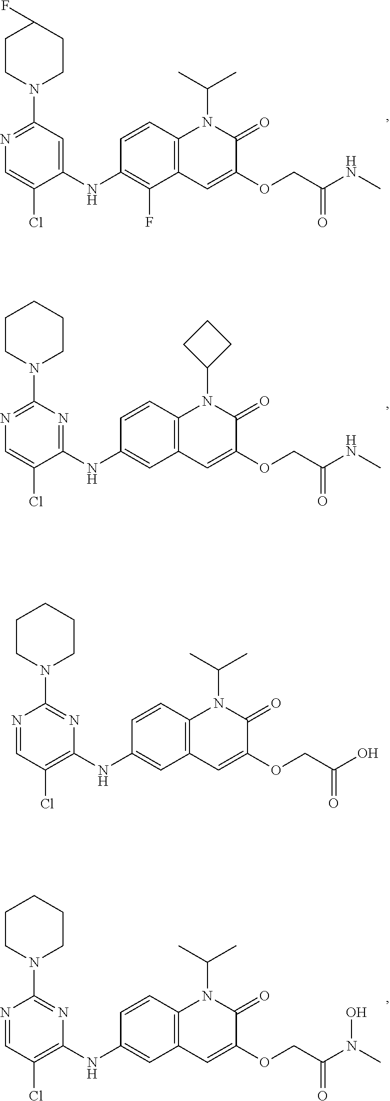 [SMILES: CNC(=O)COc1cc2c(F)c(Nc3cc(N4CCC(F)CC4)ncc3Cl)ccc2n(C(C)C)c1=O ; CNC(=O)COc1cc2cc(Nc3nc(N4CCCCC4)ncc3Cl)ccc2n(C2CCC2)c1=O ; CC(C)n1c(=O)c(OCC(=O)O)cc2cc(Nc3nc(N4CCCCC4)ncc3Cl)ccc21 ; CC(C)n1c(=O)c(OCC(=O)N(C)O)cc2cc(Nc3nc(N4CCCCC4)ncc3Cl)ccc21]

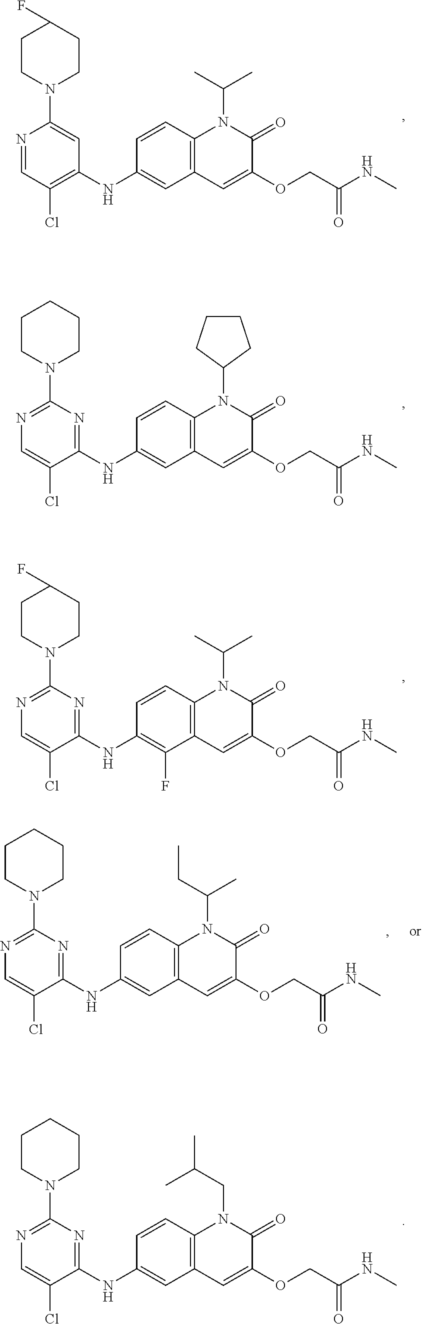

In some embodiments, the compound of the disclosure has a PTM that is:

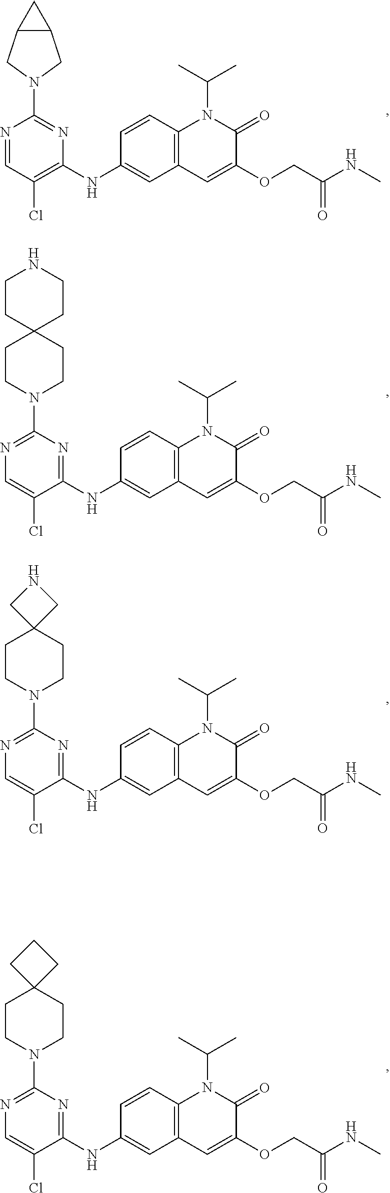 [SMILES: CNC(=O)COc1cc2cc(Nc3nc(N4CC5CC5C4)ncc3Cl)ccc2n(C(C)C)c1=O ; CNC(=O)COc1cc2cc(Nc3nc(N4CCC5(CCNCC5)CC4)ncc3Cl)ccc2n(C(C)C)c1=O ; CNC(=O)COc1cc2cc(Nc3nc(N4CCC5(CC4)CNC5)ncc3Cl)ccc2n(C(C)C)c1=O ; CNC(=O)COc1cc2cc(Nc3nc(N4CCC5(CCC5)CC4)ncc3Cl)ccc2n(C(C)C)c1=O]

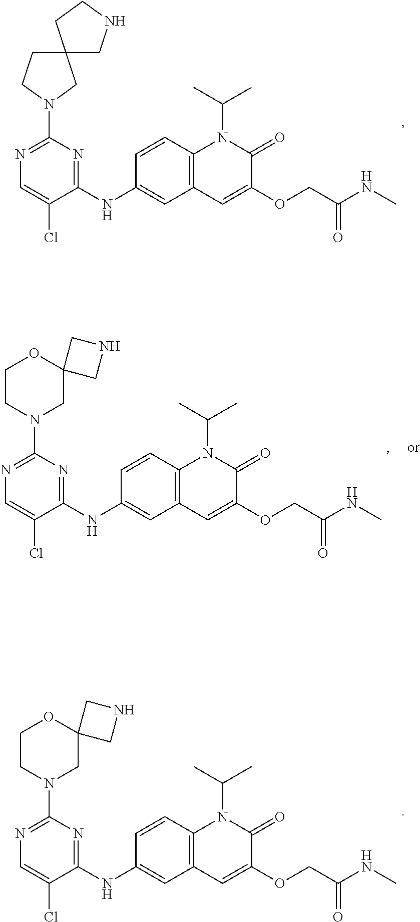 [SMILES: CNC(=O)COc1cc2cc(Nc3nc(N4CCC5(CCNC5)C4)ncc3Cl)ccc2n(C(C)C)c1=O ; CNC(=O)COc1cc2cc(Nc3nc(N4CCOC5(CNC5)C4)ncc3Cl)ccc2n(C(C)C)c1=O ; CNC(=O)COc1cc2cc(Nc3nc(N4CCOC5(CNC5)C4)ncc3Cl)ccc2n(C(C)C)c1=O]

In some embodiments, the compound of the disclosure has a PTM that is:

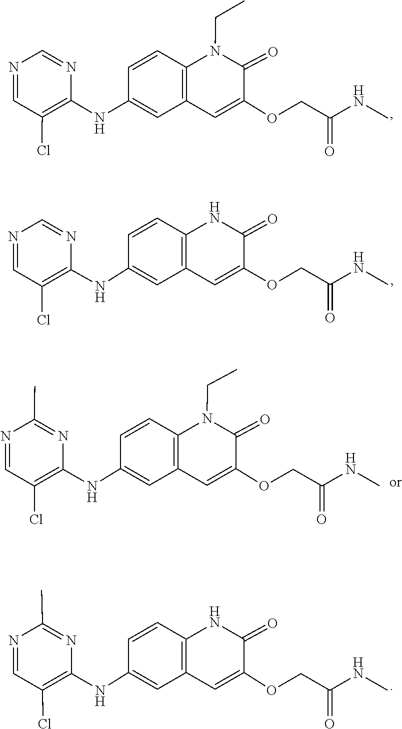 [SMILES: CCn1c(=O)c(OCC(=O)NC)cc2cc(Nc3ncncc3Cl)ccc21 ; CNC(=O)COc1cc2cc(Nc3ncncc3Cl)ccc2[nH]c1=O ; CCn1c(=O)c(OCC(=O)NC)cc2cc(Nc3nc(C)ncc3Cl)ccc21 ; CNC(=O)COc1cc2cc(Nc3nc(C)ncc3Cl)ccc2[nH]c1=O]

In some embodiments, the compound of the disclosure has a PTM that is:

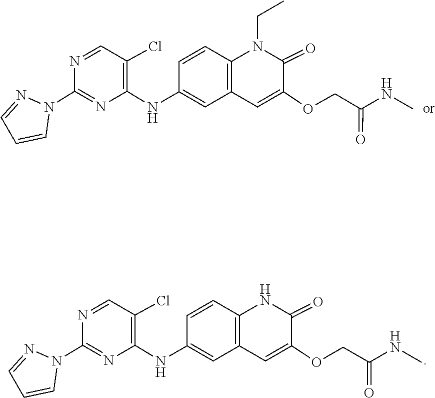 [SMILES: CCn1c(=O)c(OCC(=O)NC)cc2cc(Nc3nc(-n4cccn4)ncc3Cl)ccc21 ; CNC(=O)COc1cc2cc(Nc3nc(-n4cccn4)ncc3Cl)ccc2[nH]c1=O]

In some embodiments, the compound of the disclosure has a CLM that is:

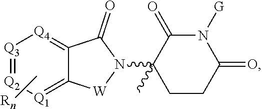

wherein:
W is CH₂, O, CH(C₁₋₃ alkyl) (e.g., CH(CH₃)), or C═O;
G is H, methyl, or OH;
each of Q₁, Q₂, Q₃, and Q₄ independently represent N, CH, or CR;
A is H, unsubstituted or substituted linear or branched alkyl, Cl, or F;
n is an integer from 1 to 4;
R is bond, H, —OR′, —NR′R″, —CR′R″—, -unsubstituted or substituted linear or branched C1-C6 linear or branched alkyl (e.g. C1-C3 alkyl and/or optionally substituted with one or more halogen), unsubstituted or substituted alkoxyl group (e.g., a methoxy, ethoxy, butoxy, propoxy, pentoxy, or hexoxy; wherein the alkoxyl optionally substituted with one or more halogen, C1-C3 alkyl, haloalkyl, or C1-C3 fluoroalkyl), optionally substituted 4-6 membered cycloalkyl, optionally substituted 4-6 membered heterocyclalkyl, —Cl, —F, —Br, —I, —CF₃, —CN, or —NO₂, wherein one R is covalently joined to the L;
R′ and R″ are each independently selected from bond H, and substituted or unsubstituted C1-C4 alkyl (e.g., methyl or ethyl);
[symbol] represents a bond that may be stereospecific ((R) or (S)) or non-stereospecific.

In some embodiments, the compound of the disclosure has a CLM that is:

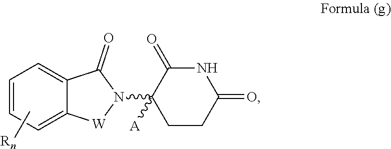

wherein:
W is CH₂, O, CH(C₁₋₃ alkyl) (e.g., CH(CH₃)), or C═O;
A is a H, methyl, or optionally substituted linear or branched alkyl;
n is an integer from 1 to 4;
R is independently selected from a H, O, OH, N, NH, NH₂, methyl, optionally substituted linear or branched alkyl (e.g., optionally substituted linear or branched C1-C6 alkyl), C1-C6 alkoxy, and -alkyl-aryl (e.g., an -alkyl-aryl comprising at least one of C1-C6 alkyl, C4-C7 aryl, or a combination thereof), aryl (e.g., C5-C7 aryl), amine, amide, or carboxy), wherein one R or W is optionally modified to be covalently joined to a chemical linker group (L) and
[symbol] of Formula (g) represents a bond that may be stereospecific ((R) or (S)) or non-stereospecific.

In some embodiments, A is H.

In some embodiments, W is CH₂ or C═O.

In some embodiments, W is CH₂.

In some embodiments, W is C═O.

In some embodiments, the compound of the disclosure has a L that is selected from:

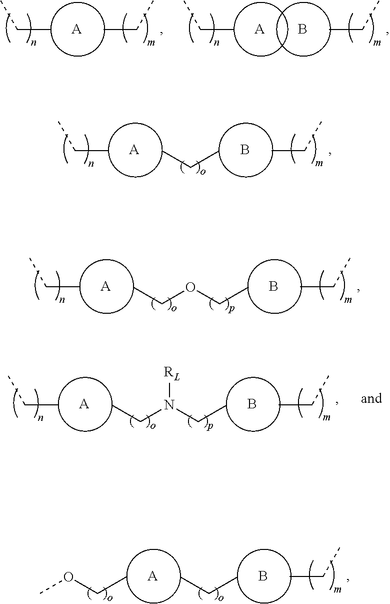

wherein:

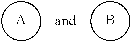

and are each independently a 3-7 membered cycloalkyl or a 3-7 membered heterocycloalkyl (e.g., 4-6 membered cycloalkyl or 4-6 membered heterocycloalkyl), wherein overlapping circles indicates spirocyclic rings;
each m, n, o, and p is independently 0, 1, 2, 3, 4, 5, or 6;
R_{L} is selected from H and C1-3 alkyl;
the linker is optionally substituted with at least one of: (i) ═O and (ii) 1-4 (e.g., 1, 2, 3, or 4) substitutions independently selected from a C1-3 alkyl (e.g., methyl) and a halogen (e.g., F, Cl, or Br); and
[symbol] indicates the attachment point to the PTM or the CLM.

In some embodiments, the compound of the disclosure has a L that is selected from:

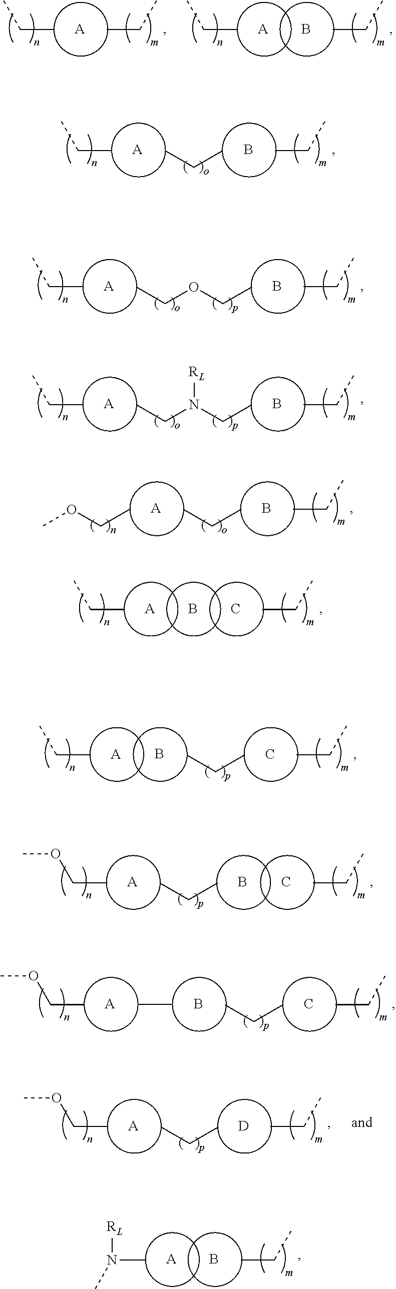

wherein:

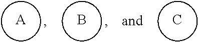

and are each independently a 3-7 membered cycloalkyl or a 3-7 membered heterocycloalkyl (e.g., 4-6 membered cycloalkyl or 4-6 membered heterocycloalkyl), wherein overlapping circles indicates spirocyclic rings;

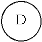

is a 8-10 membered bridged cycloalkyl, a 8-10 membered bridge heterocycloalkyl, a 3-7 membered heterocyclyl having one or two double bonds (e.g., 3-7 membered heterocyclyl having one or two double bonds), or a 7-10 membered fused bicyclic heterocycloalkyl (e.g., a 7-9 membered fused bicyclic heterocycloalkyl);
each m, n, o, and p is independently 0, 1, 2, 3, 4, 5, or 6;
R_{L} is selected from H and C₁₋₃ alkyl;
the linker is optionally substituted with at least one of: (i) ═O and (ii) 1-4 (e.g., 1, 2, 3, or 4) substitutions independently selected from a C1-3 alkyl (e.g., methyl), OH, and a halogen (e.g., F, Cl, or Br); and
[symbol] indicates the attachment point to the PTM or the CLM.

In some embodiments, the compound of the disclosure has a L that is selected from:

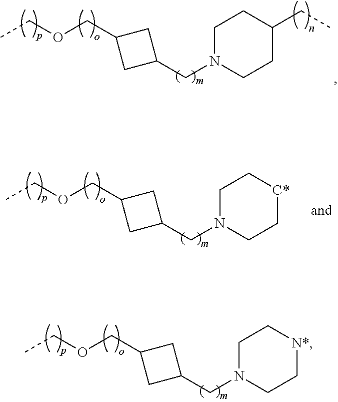

wherein:
the above chemical linker groups that do not include substitutions are optionally substituted with at least one of: (i) ═O and (ii) 1-4 (e.g., 1, 2, 3, or 4) substitutions independently selected from a C1-3 alkyl (e.g., methyl) and a halogen (e.g., F, Cl, or Br);
the * indicates an atom (e.g., a nitrogen, carbon, or oxygen) that is covalently linked to the CLM or the PTM, or that is shared with the CLM or the PTM;
[symbol] indicates the attachment point to the PTM or the CLM; and
each of m, n, o, and p is independently 0, 1, 2, 3, 4, 5, 6, 7, 8, 9, or 10 (preferably 0, 1, 2, or 3).

In some embodiments, the compound of the disclosure has a L that is selected from:

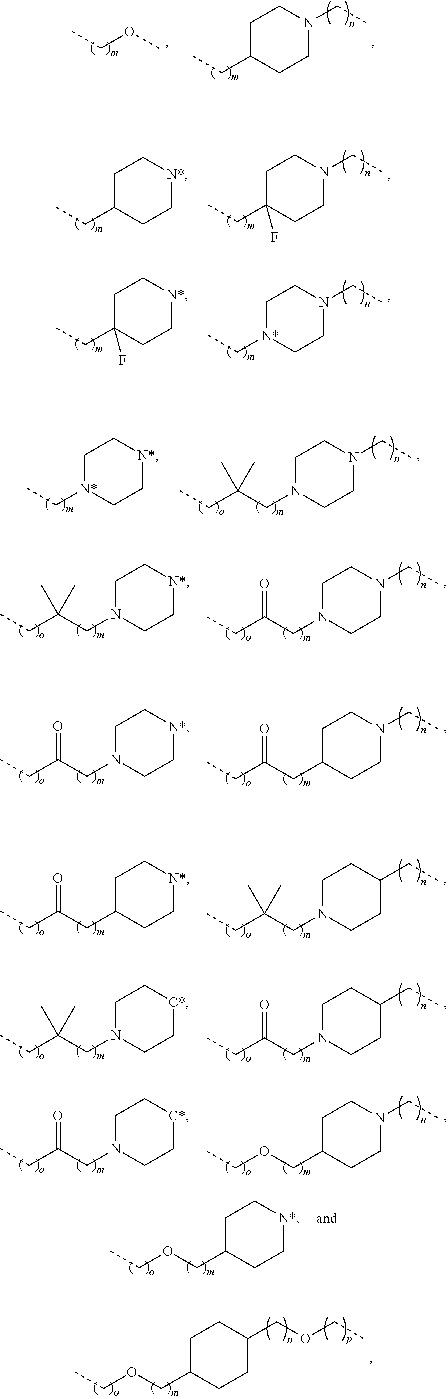

wherein:
the above chemical linker groups that do not include substitutions are optionally substituted with at least one of: (i) ═O and (ii) 1-4 (e.g., 1, 2, 3, or 4) substitutions independently selected from a C1-3 alkyl (e.g., methyl) and a halogen (e.g., F, Cl, or Br);
the * indicates an atom (e.g., a nitrogen or carbon) that is covalently linked to the CLM or the PTM, or that is shared with the CLM or the PTM;
[symbol] indicates the attachment point to the PTM or the CLM; and
each m, n, o, and p is independently 0, 1, 2, 3, 4, 5, 6, 7, 8, 9, or 10 (preferably 0, 1, 2, or 3).

In some embodiments, the compound of the disclosure has a L that is selected from:

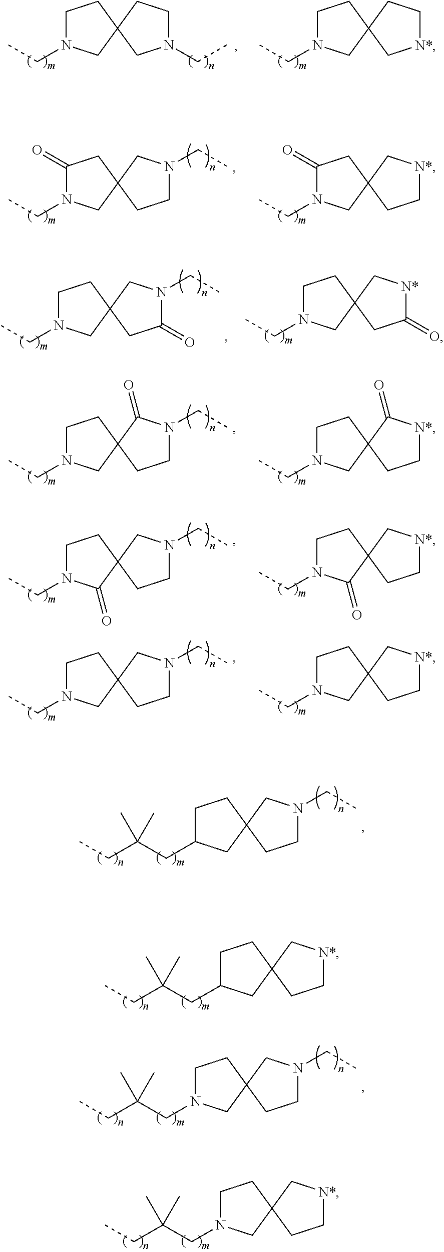

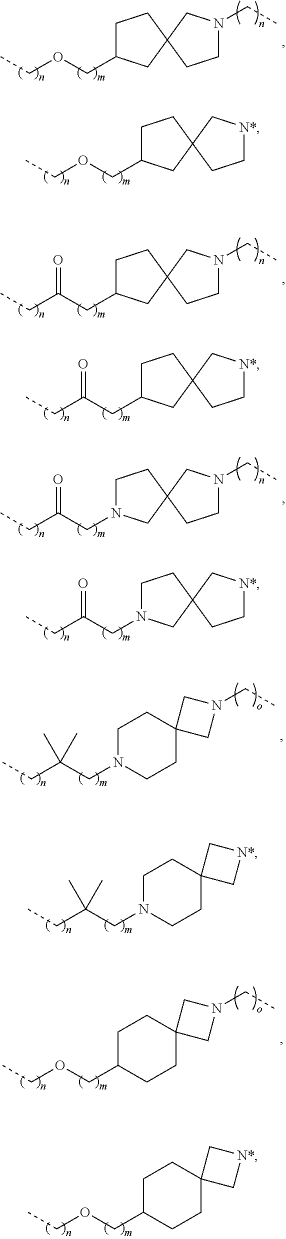

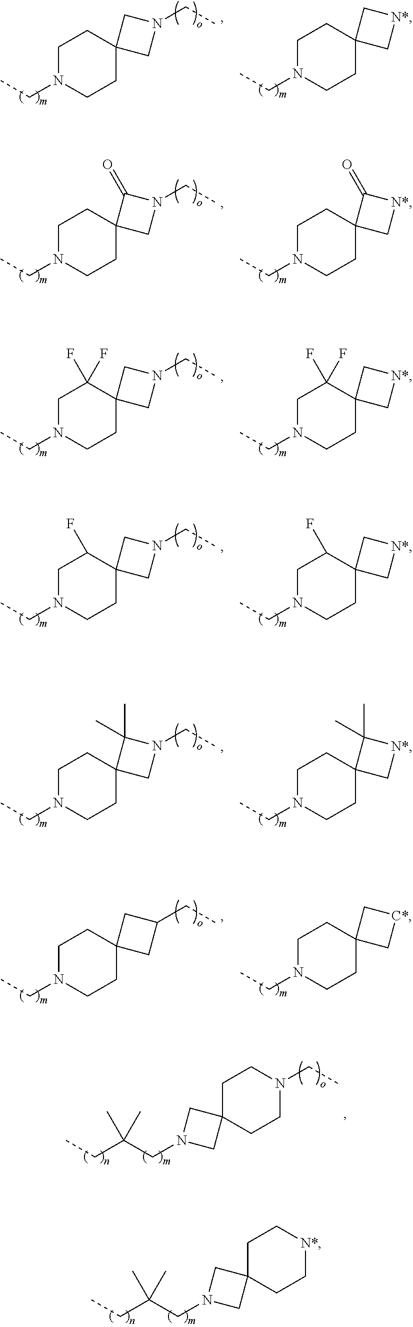

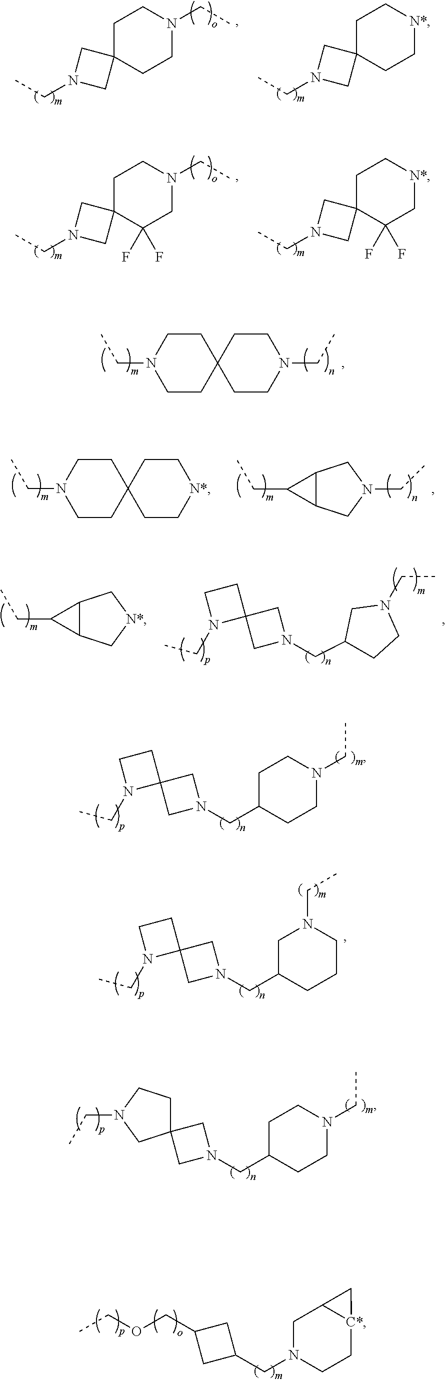

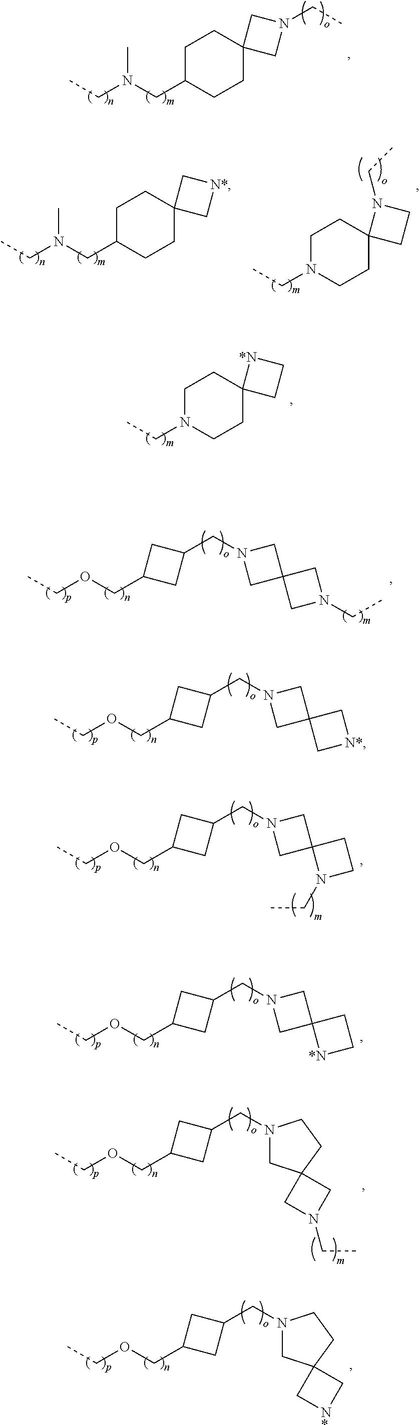

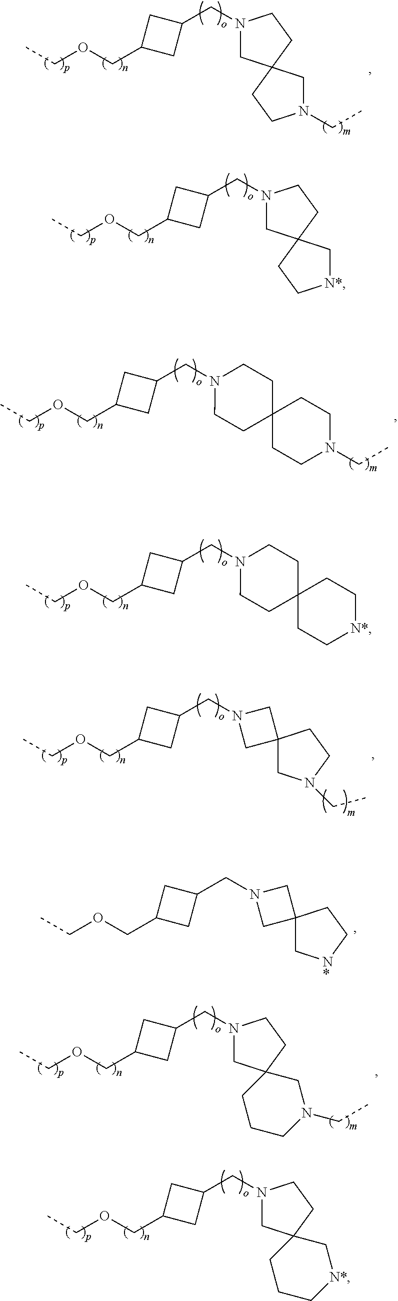

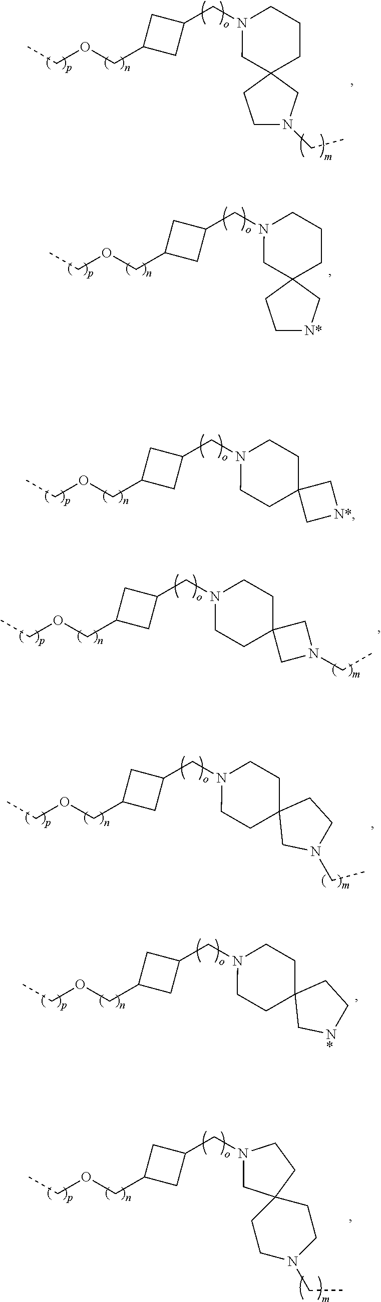

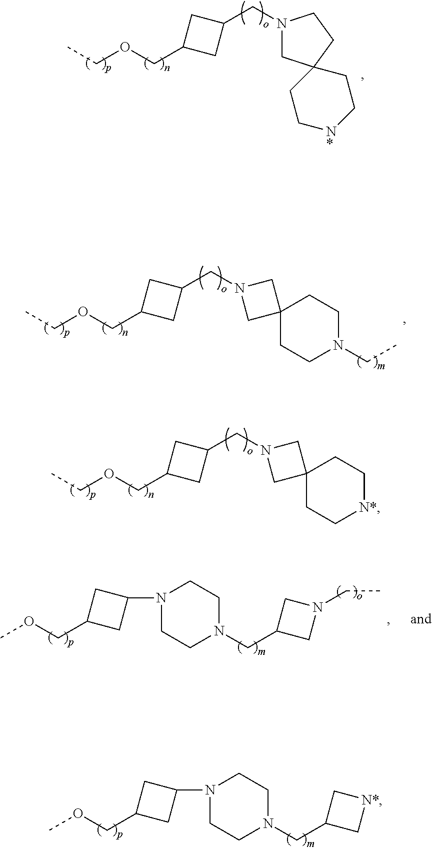

wherein:
the above chemical linker groups that do not include substitutions are optionally substituted with at least one of: (i) ═O and (ii) 1-4 (e.g., 1, 2, 3, or 4) substitutions independently selected from a C₁₋₃ alkyl (e.g., methyl) and a halogen (e.g., F, Cl, or Br);
the * indicates an atom (e.g., a nitrogen or carbon) that is covalently linked to the CLM or the PTM, or that is shared with the CLM or the PTM;
[symbol] indicates the attachment point to the PTM or the CLM; and
each m, n, and o is independently 0, 1, 2, 3, 4, 5, 6, 7, 8, 9, or 10 (preferably 0, 1, 2, or 3).

In some embodiments, the compound of the disclosure has a L that is selected from:

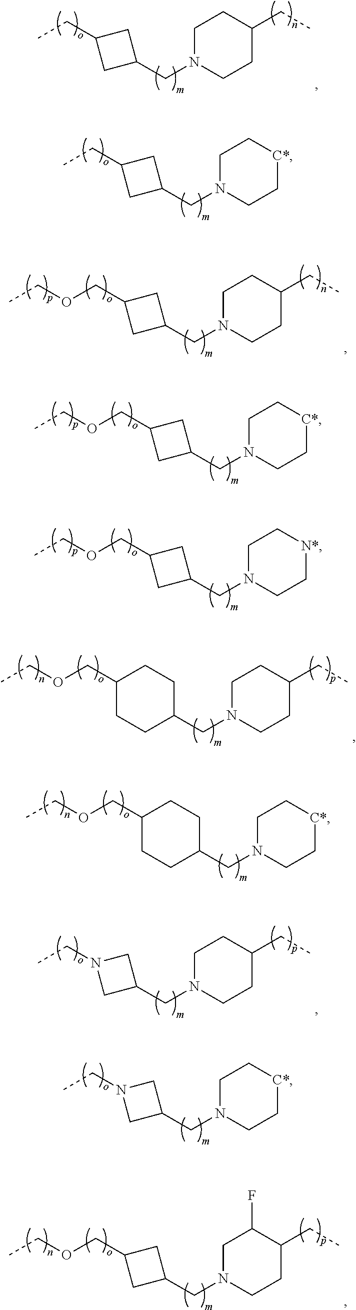

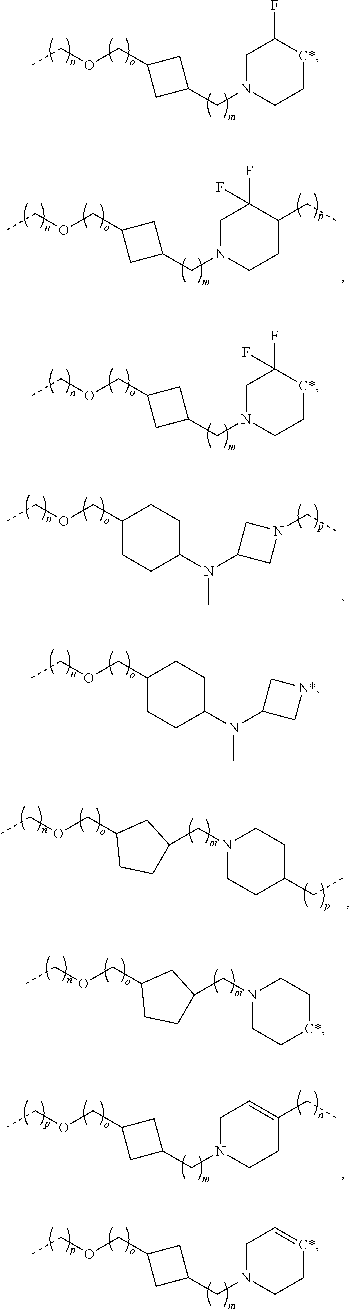

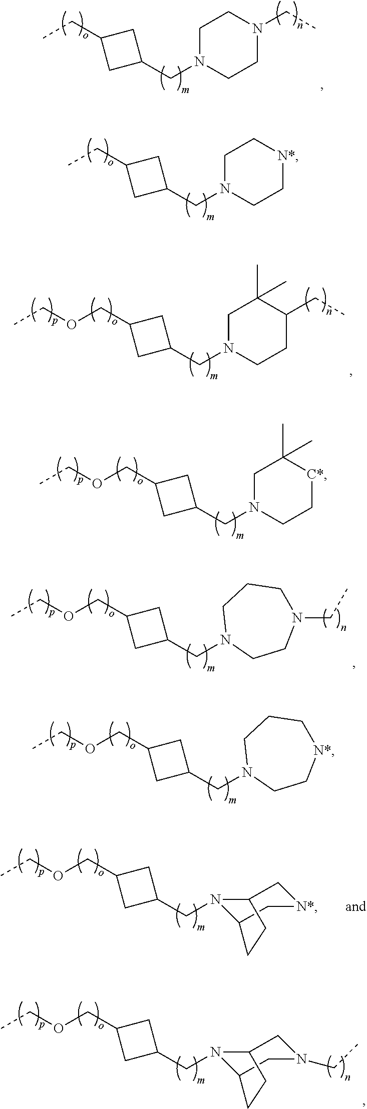

wherein:
the above chemical linker groups that do not include substitutions are optionally substituted with at least one of: (i) ═O and (ii) 1-4 (e.g., 1, 2, 3, or 4) substitutions independently selected from a C1-3 alkyl (e.g., methyl) and a halogen (e.g., F, Cl, or Br);
* indicates an atom (e.g., a nitrogen or carbon) that is shared with or covalently linked to the CLM or the PTM;
[symbol] indicates the attachment point to the PTM or the CLM; and
each m, n, o, and p is independently 0, 1, 2, 3, 4, 5, 6, 7, 8, 9, or 10 (preferably 0, 1, 2, or 3).

In some embodiments, the compound of the disclosure has a L that is selected from:

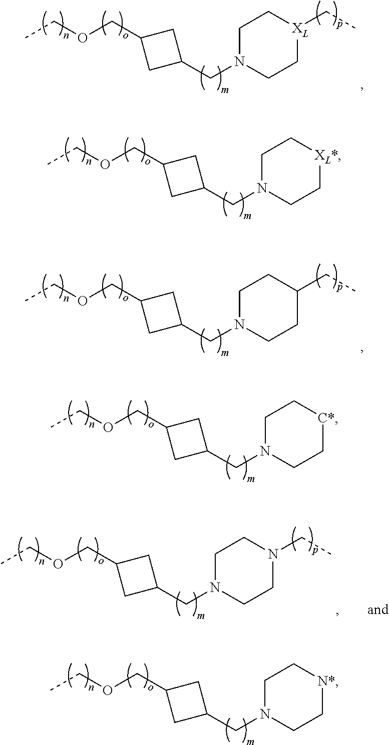

wherein:
X_{L} is a N or CH group;
each m, n, o, and p is independently 0, 1, 2, 3, 4, 5, 6, 7, 8, 9, or 10 (preferably 0, 1, 2, or 3);
* indicates an atom (e.g., a nitrogen or carbon) that is shared with or covalently linked to the CLM or the PTM;
[symbol] indicates the attachment point to the PTM or the CLM; and
the chemical linker includes 0-4 substitutions (preferably 0, 1, or 2 substitutions), each substitution independently a C1-3 alkyl (preferably, methyl).

In some embodiments, the compound of the disclosure has a L that is selected from:

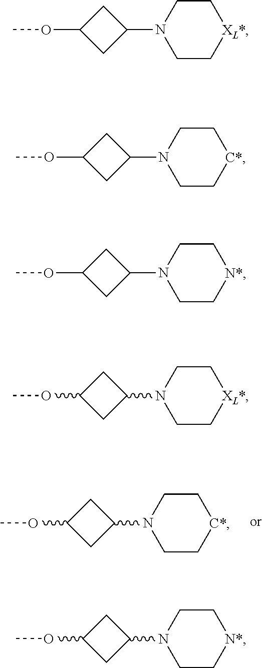

wherein:
X_{L} is N or CH group;
[symbol] represents a stereospecific bond, wherein one has an (R) configuration and the other has an (S) configuration;
* indicates an atom (e.g., a nitrogen or carbon) that is shared with or covalently linked to the CLM or the PTM;
[symbol] indicates the attachment point to the PTM or the CLM; and
the chemical linker includes 0-4 substitutions (preferably 0, 1, or 2 substitutions), each substitution independently a C₁₋₃ alkyl (preferably, methyl).

In some embodiments, the compound of the disclosure has:
(a) a CLM that is

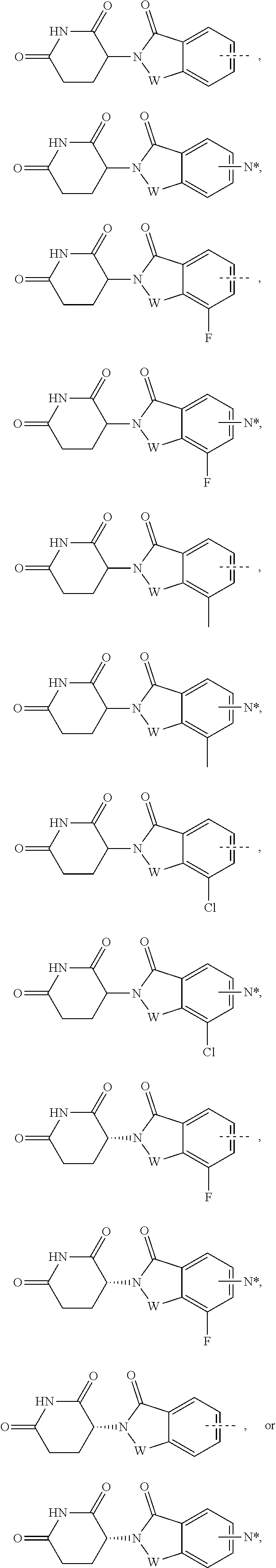

wherein:
   [symbol] of the CLM indicates the point of attachment with the L; and
   N* is a nitrogen atom that is shared with the chemical linker group;
(b) a PTM that is

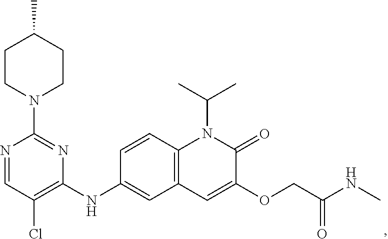

wherein [symbol] of the PTM indicates the point of attachment with the L; and/or
(c) the L is selected from

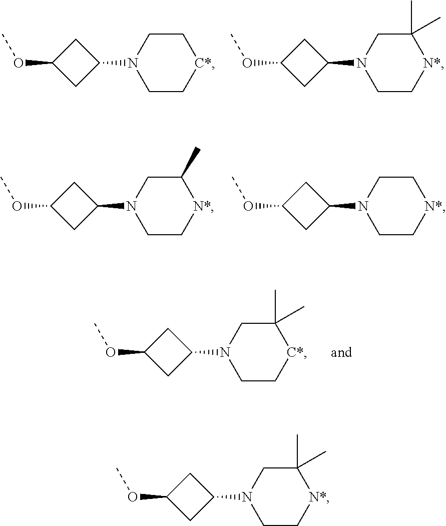

wherein * indicates an atom (e.g., a carbon or nitrogen) that is covalently linked to the CLM or PTM, or that is shared with the CLM or PTM, and each of [symbol] indicates the point of attachment with the CLM or the PTM.

In some embodiments, the compound of the disclosure has:
(a) a CLM that is

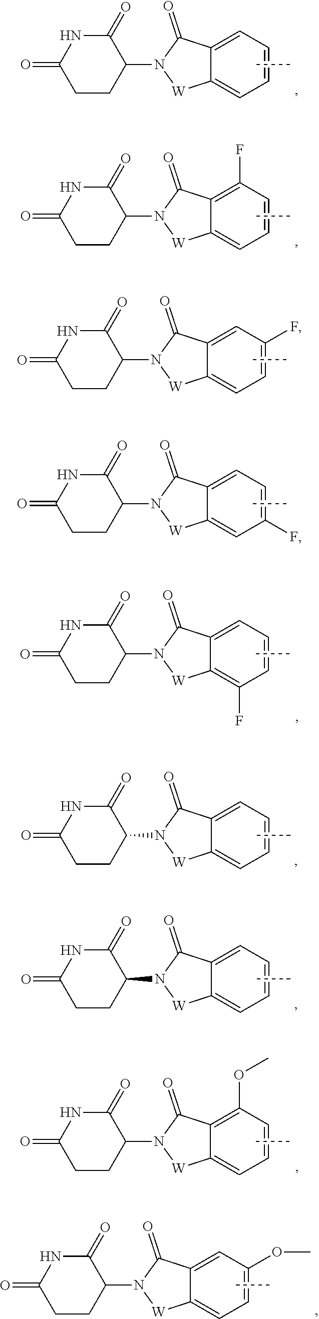

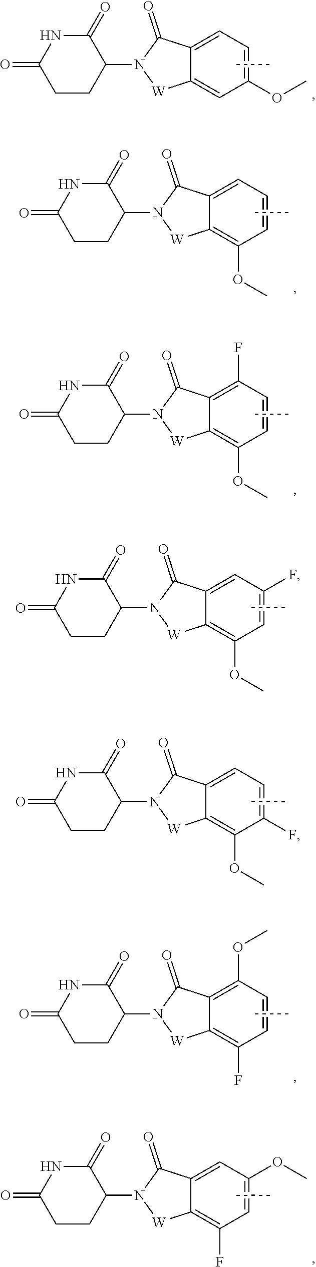

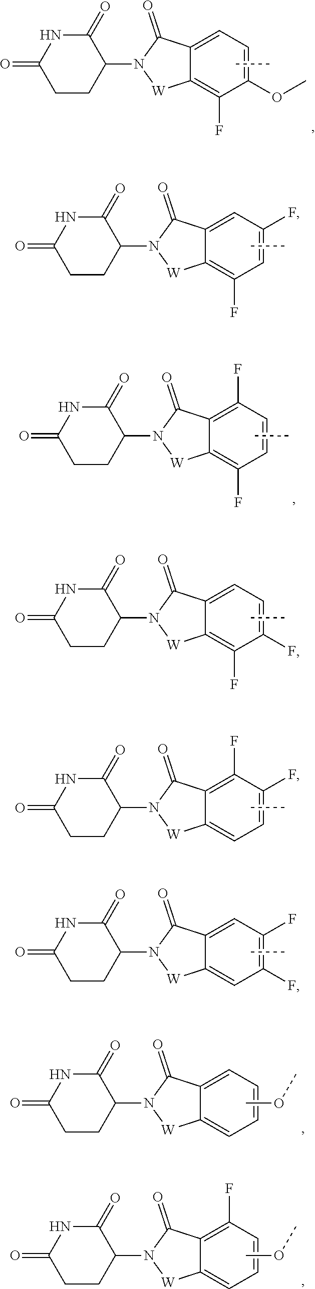

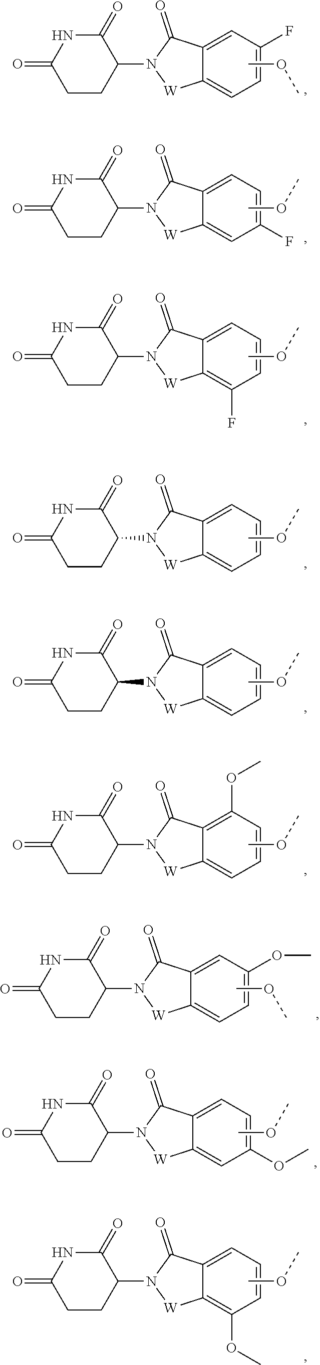

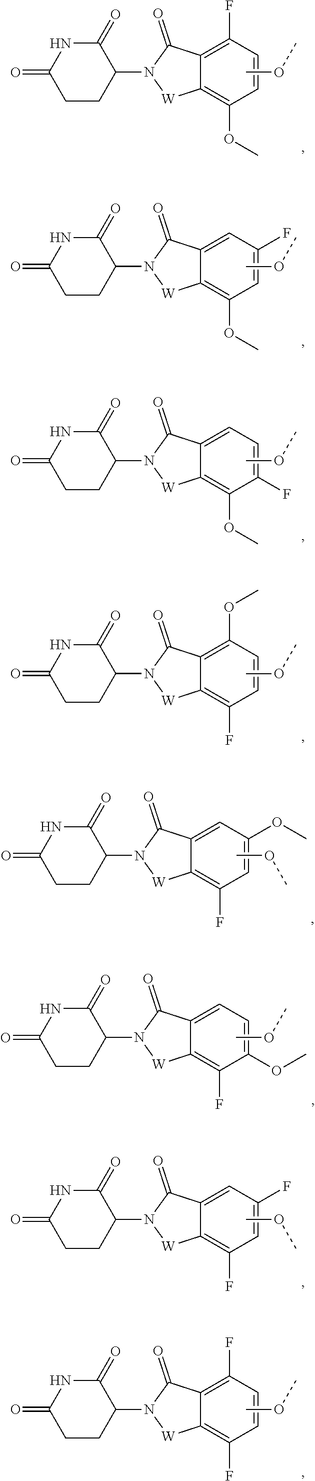

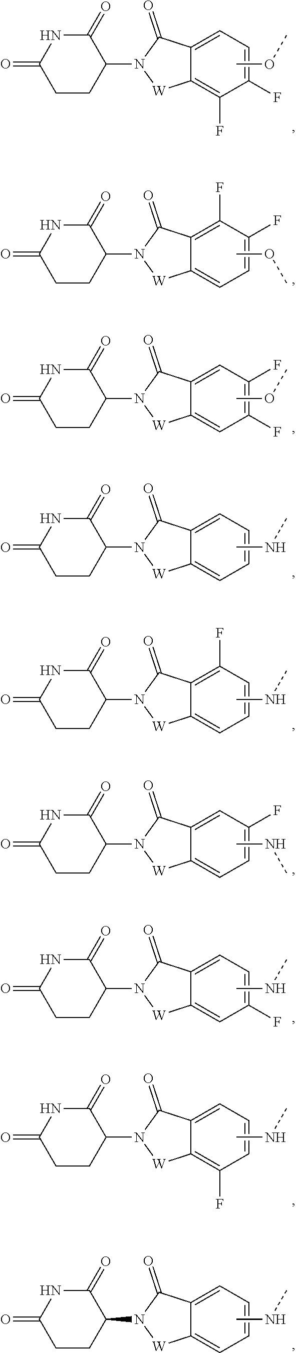

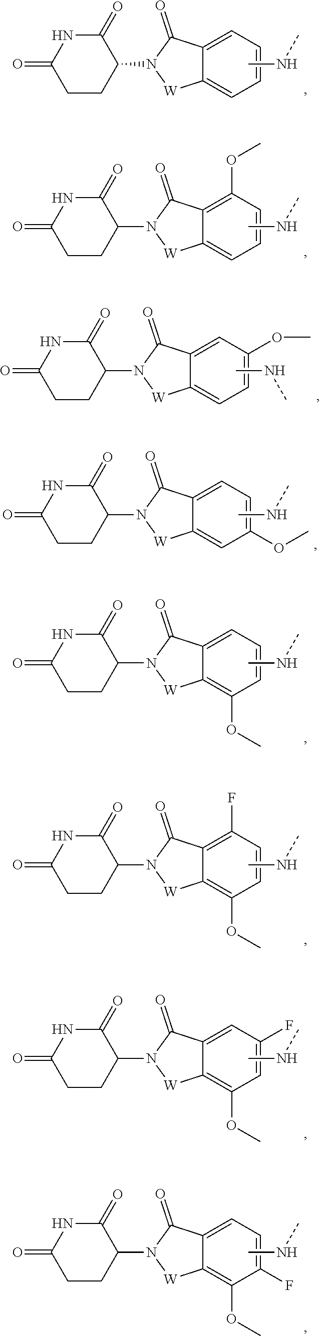

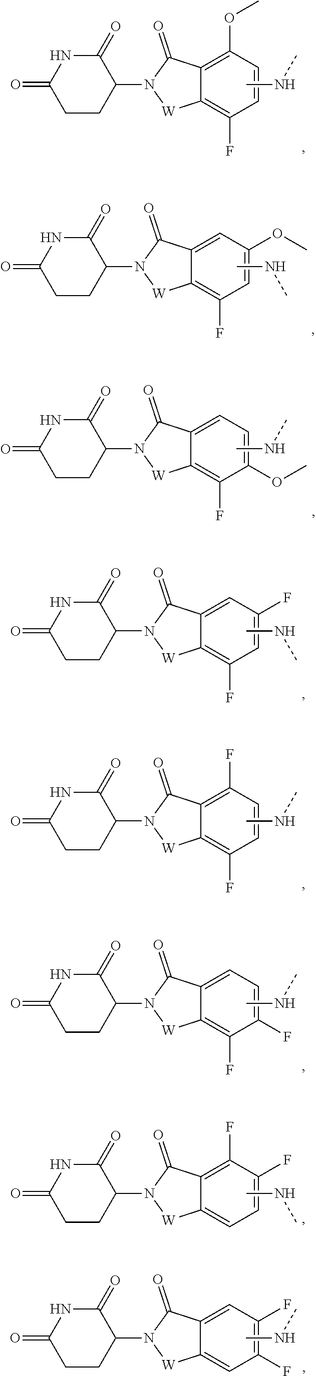

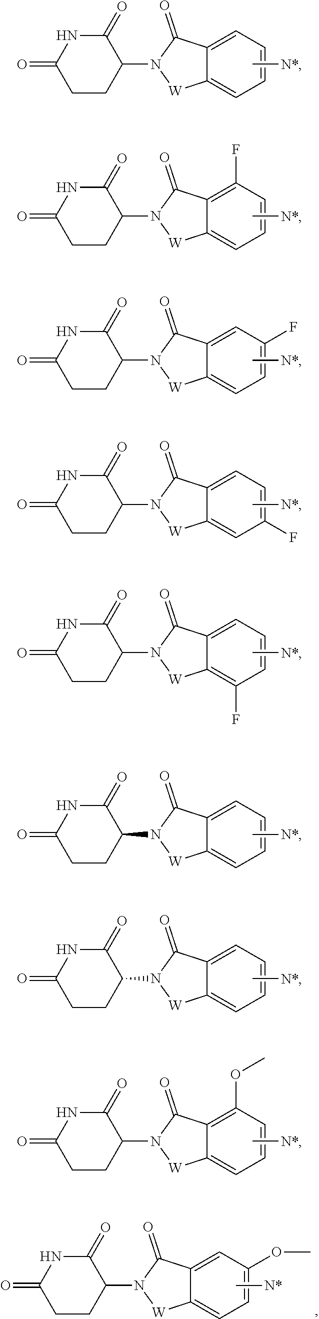

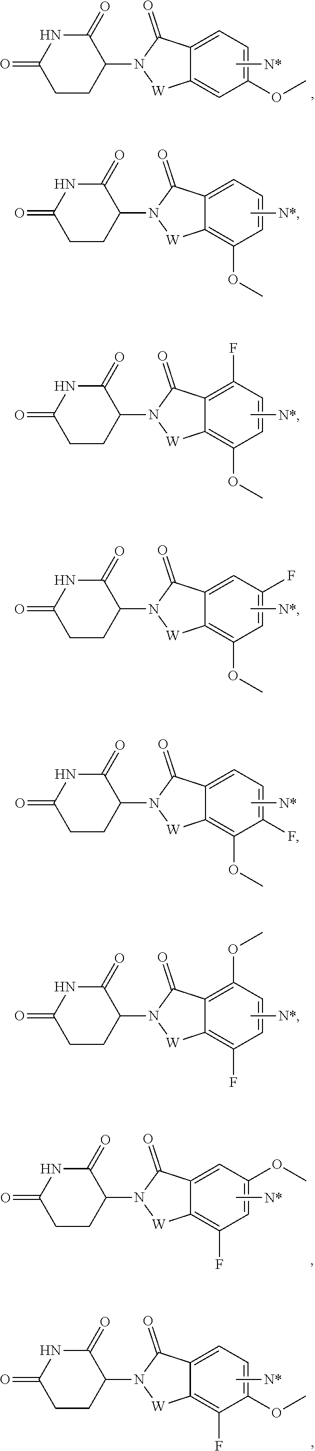

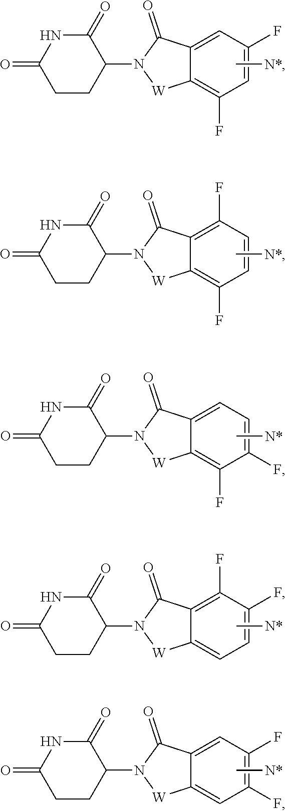

wherein:
   [symbol] of the CLM indicates the point of attachment with the L; and
   N* is a nitrogen atom that is shared with the chemical linker group;
(b) a PTM that is:

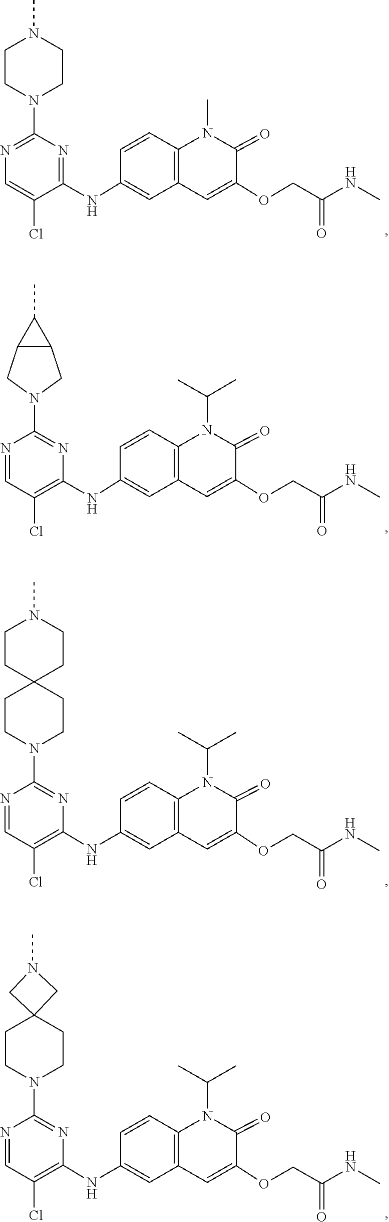

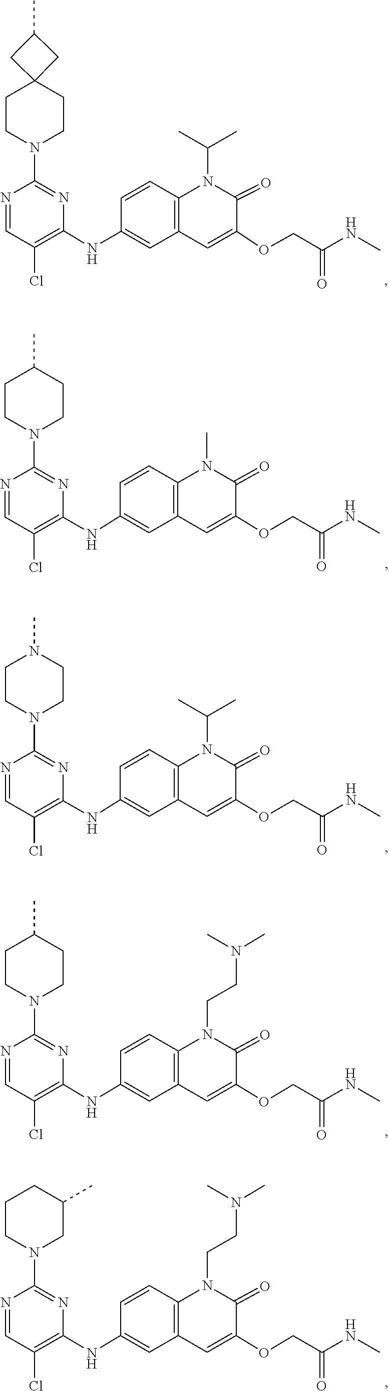

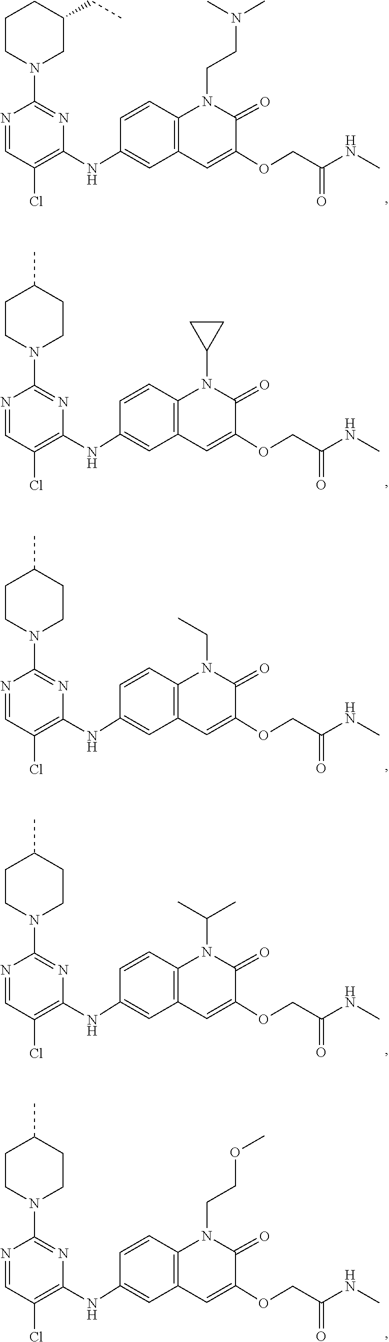

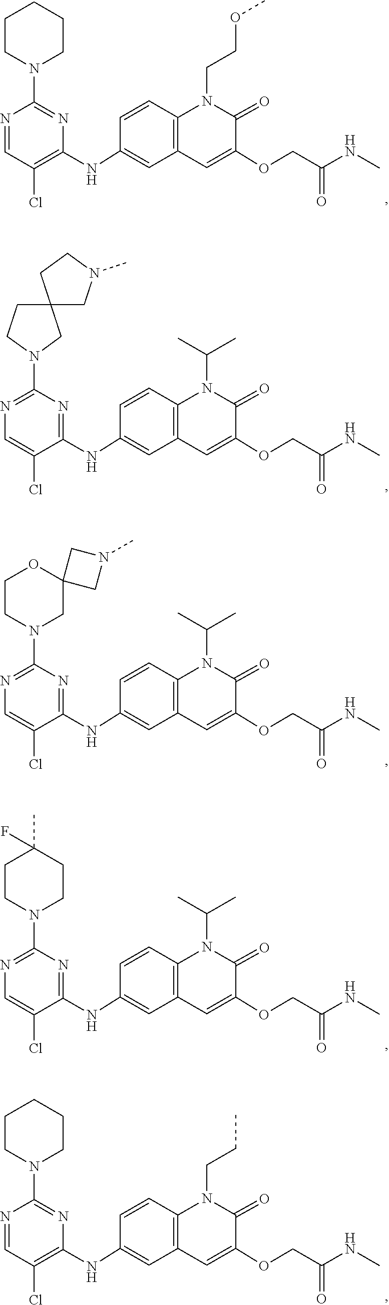

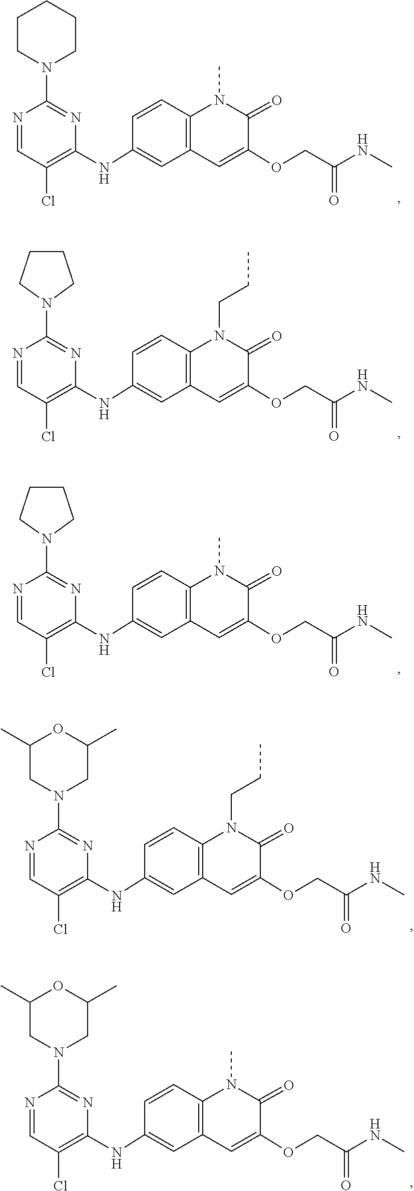

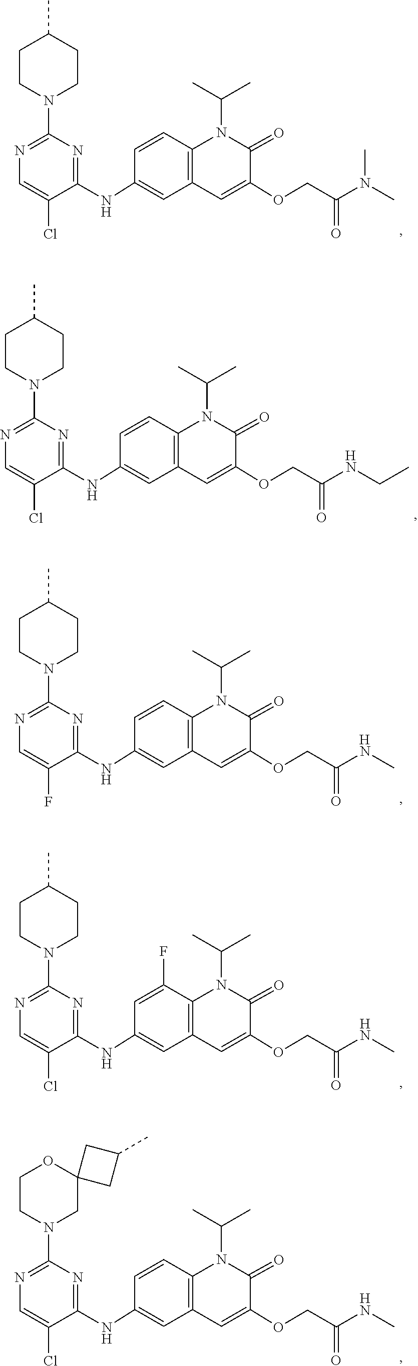

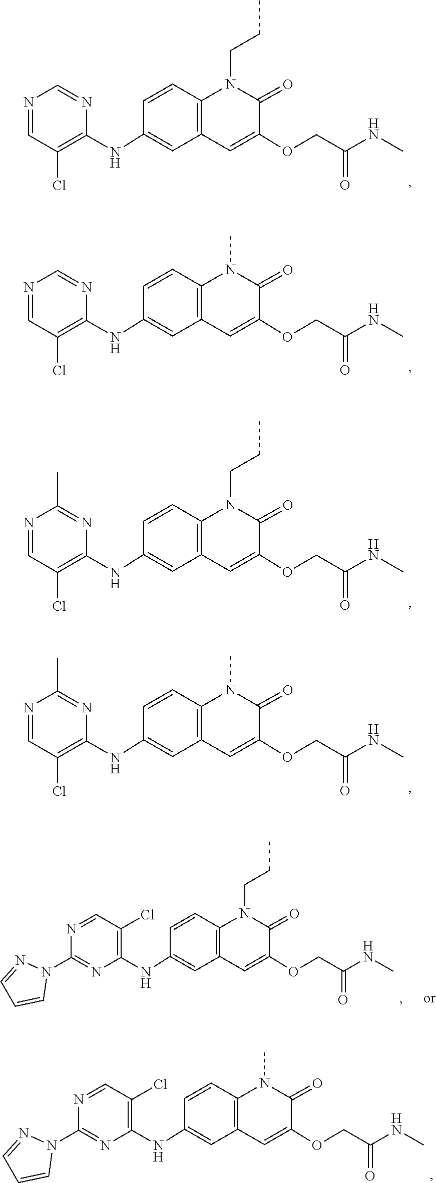

wherein [symbol] of the PTM indicates the point of attachment with the L;
(c) a L that is selected from:

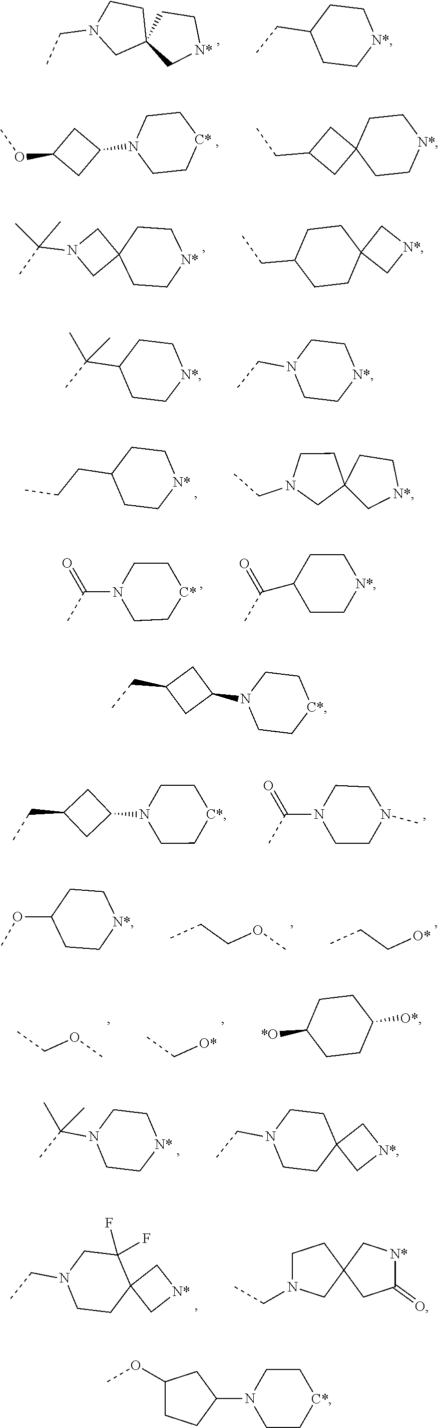

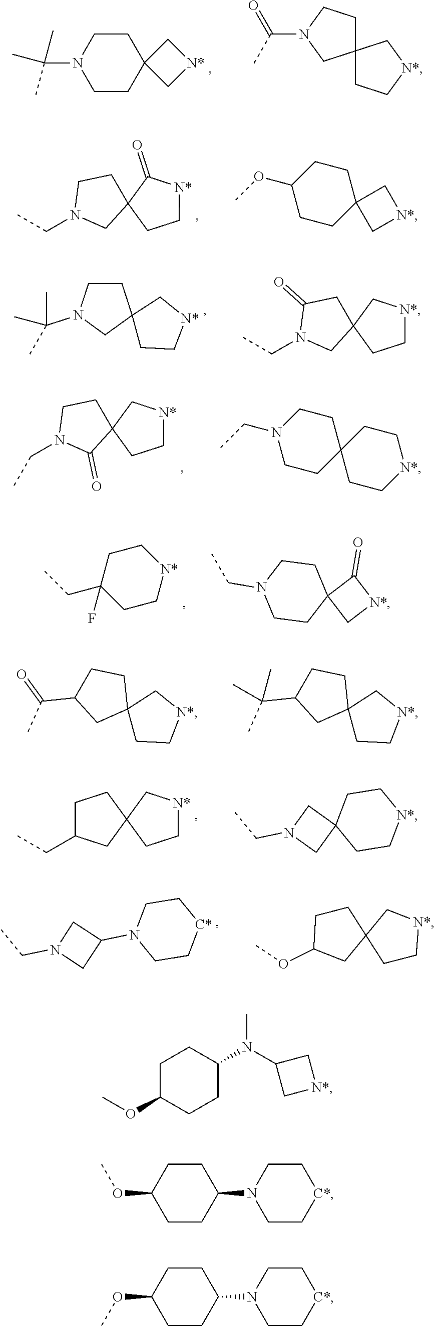

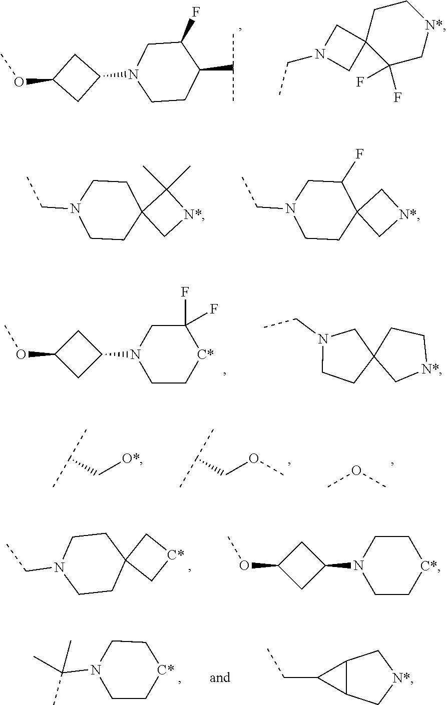

wherein * indicates an atom (e.g., a carbon, nitrogen, or oxygen) that is covalently linked to the CLM or PTM, or that is shared with the CLM or PTM, and each of [symbol] , [symbol] , [symbol] , and [symbol] indicates the point of attachment with the CLM or the PTM; or
(d) any combination of (a), (b), and (c).

In some embodiments, the compound of the disclosure has:
(a) a CLM that is:

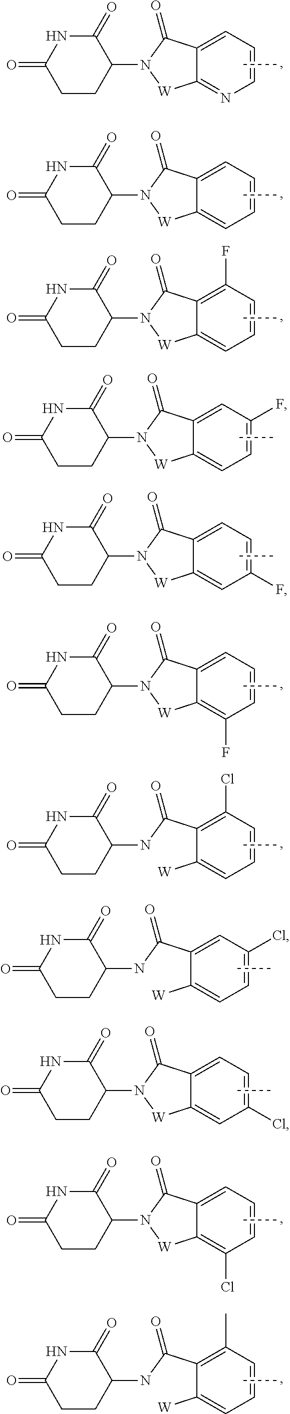

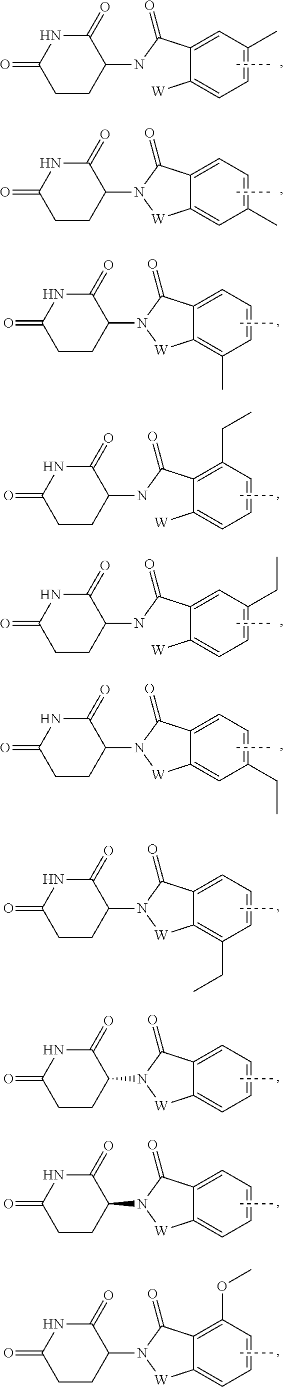

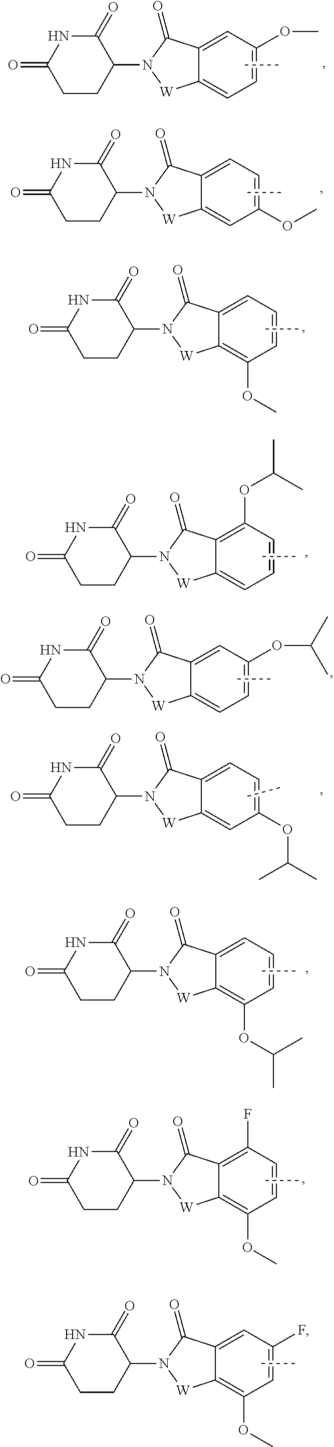

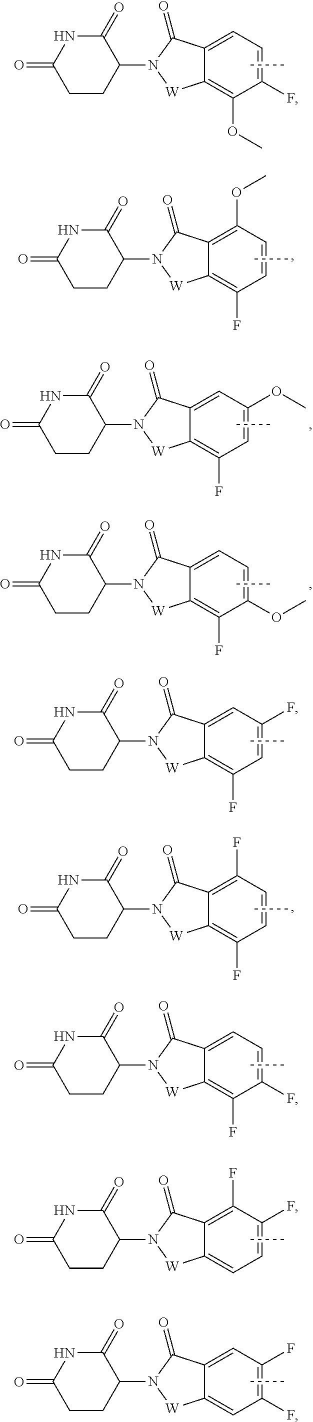

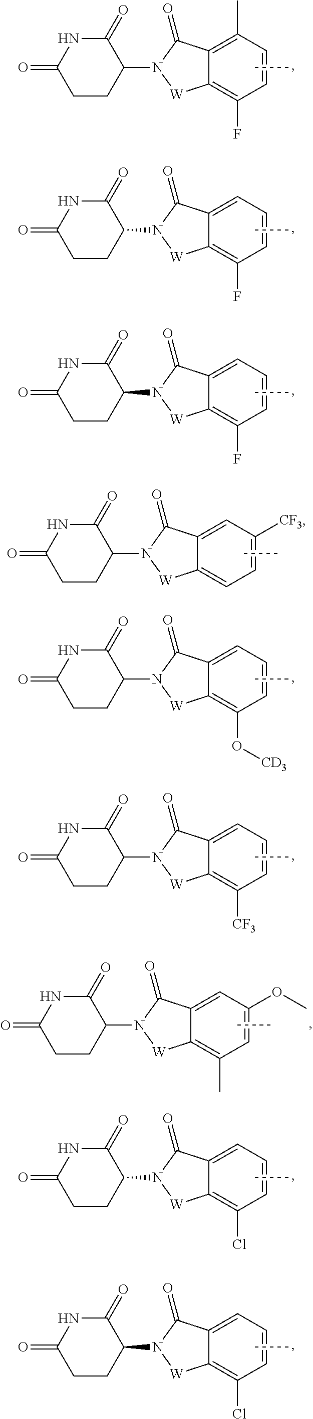

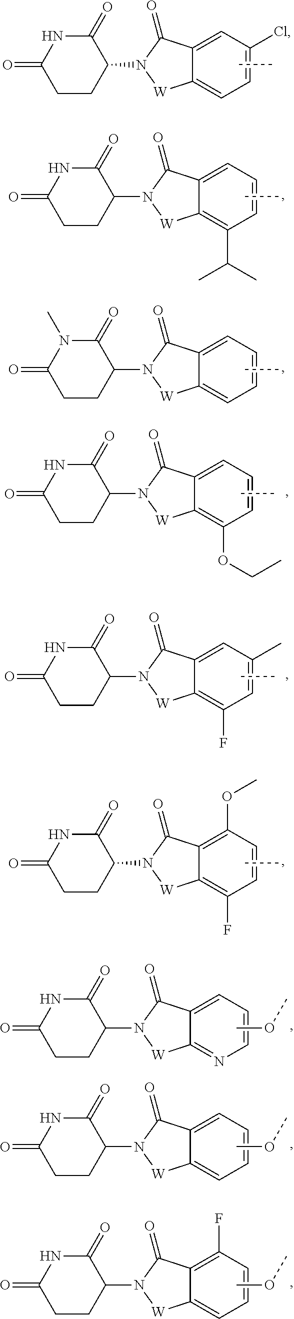

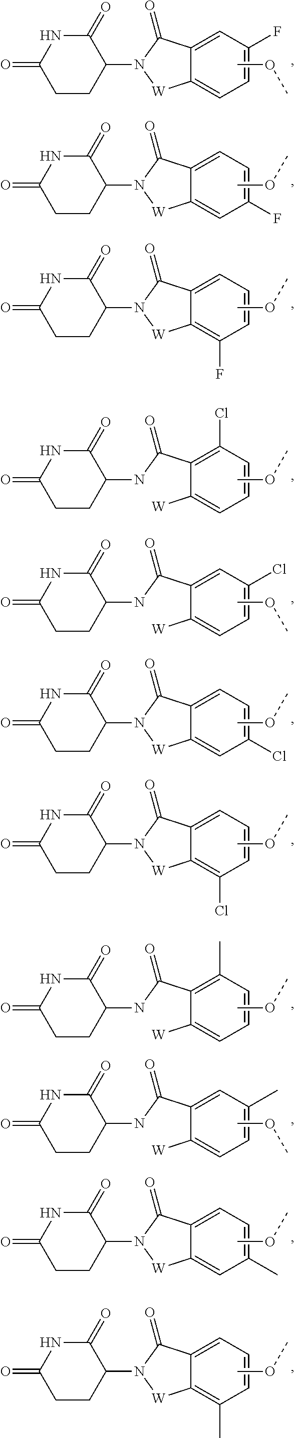

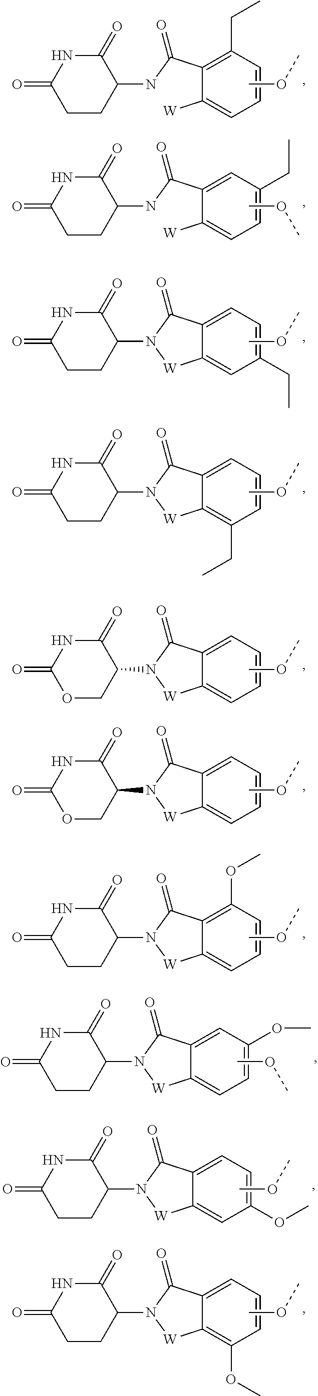

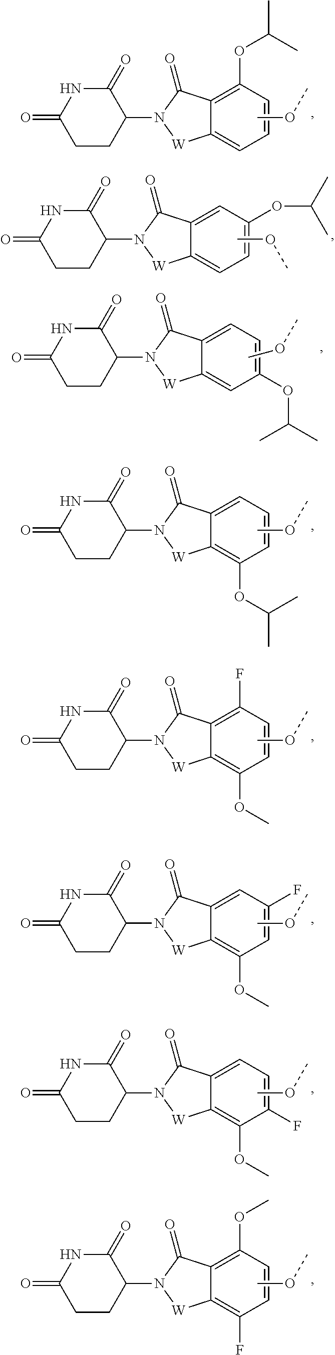

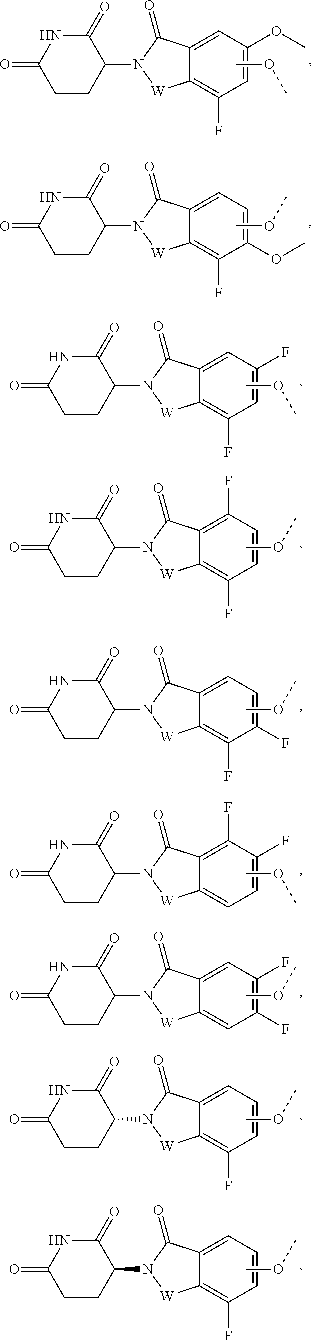

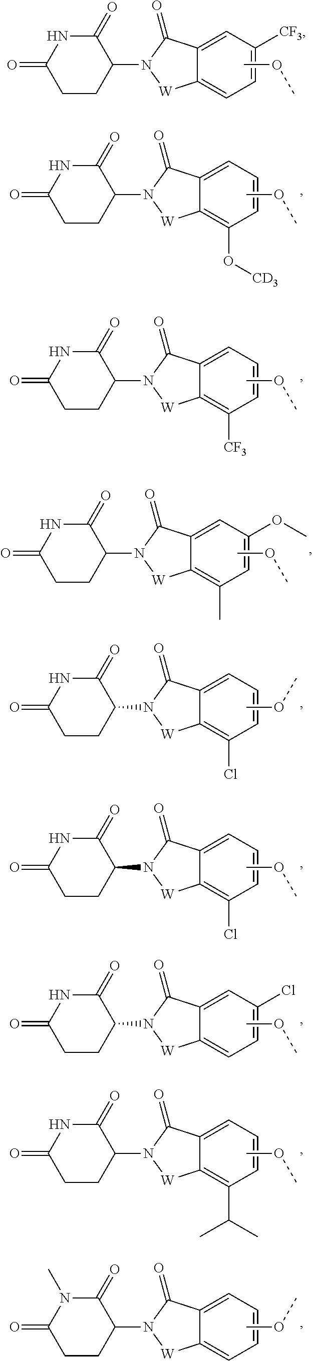

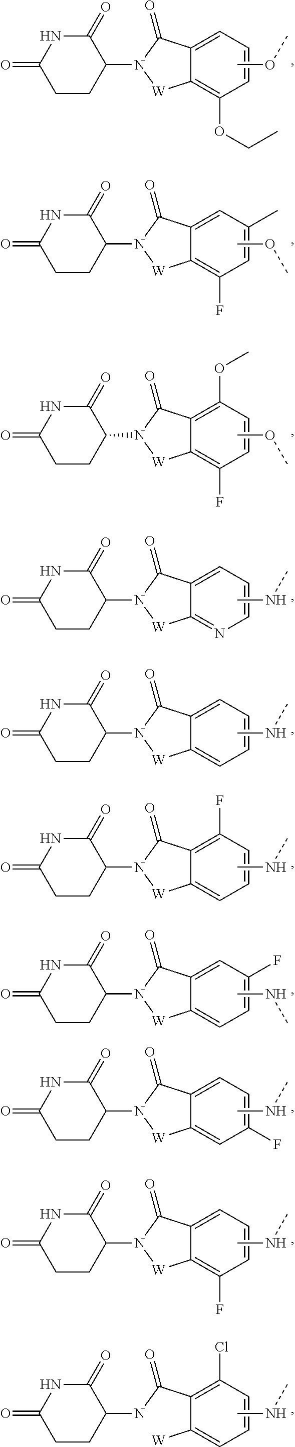

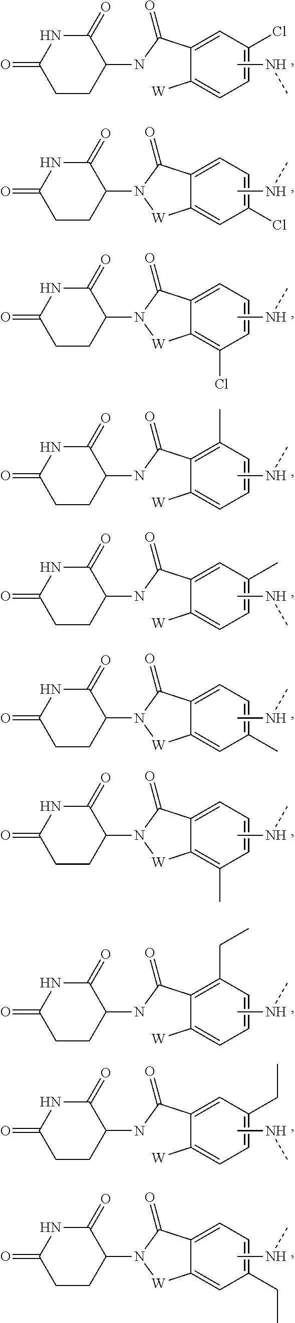

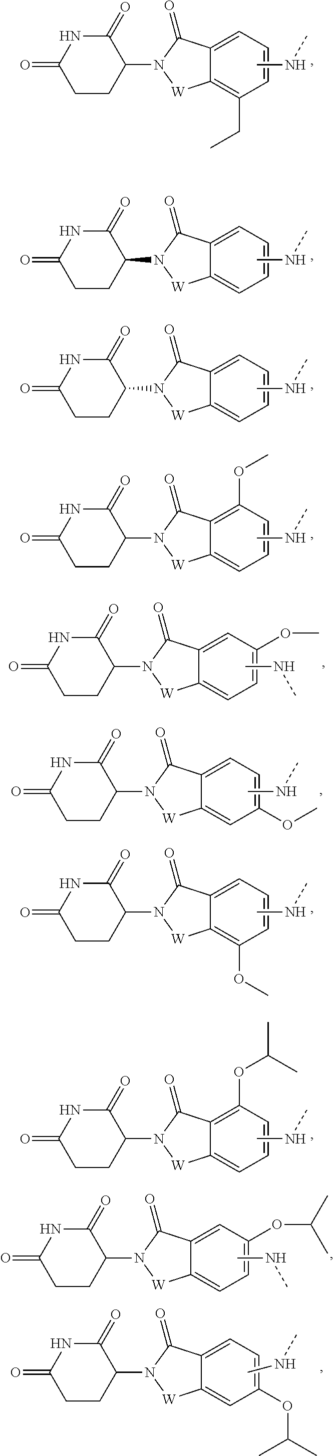

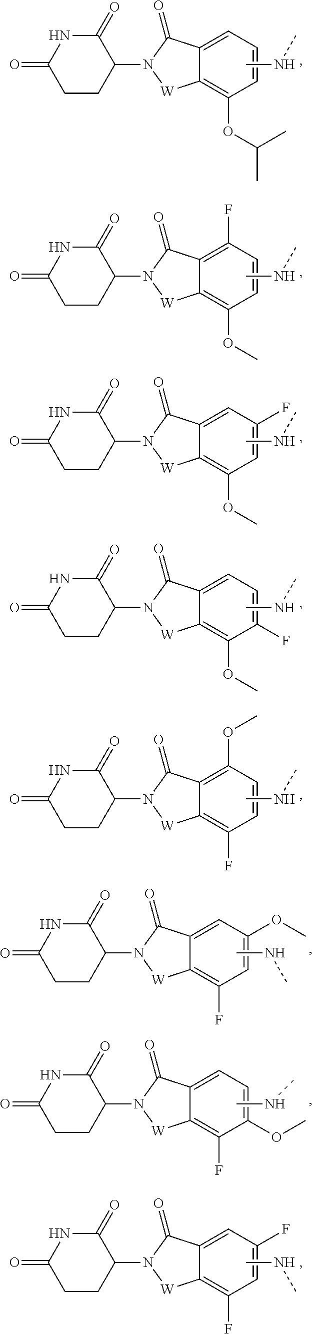

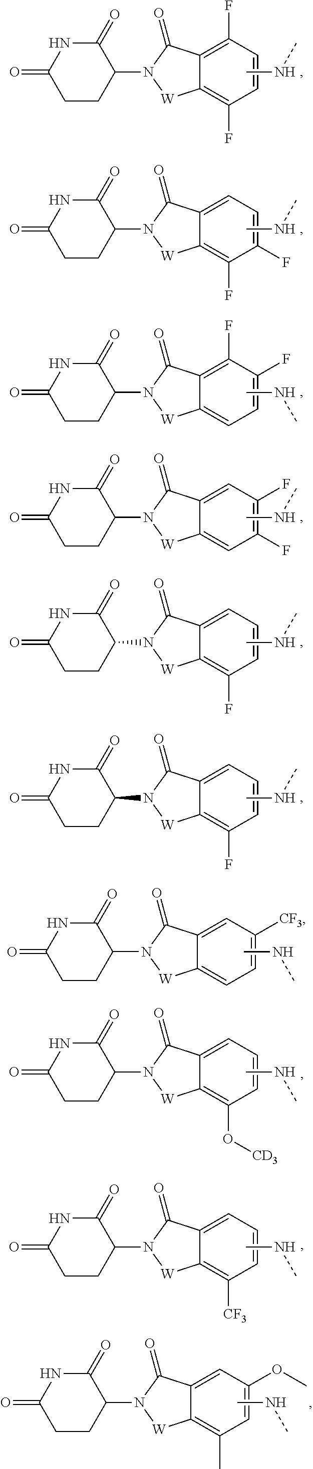

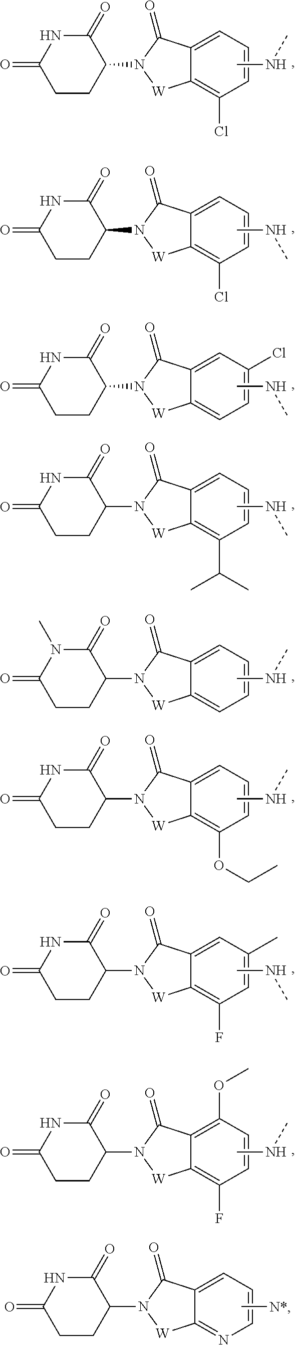

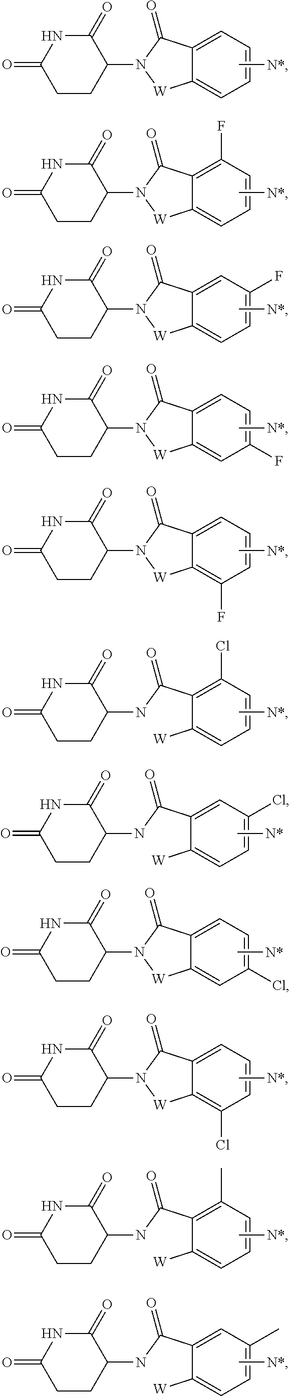

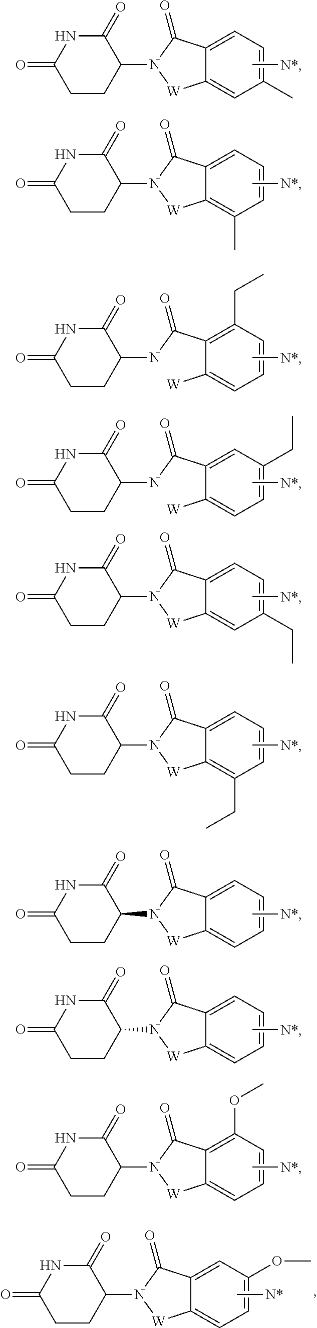

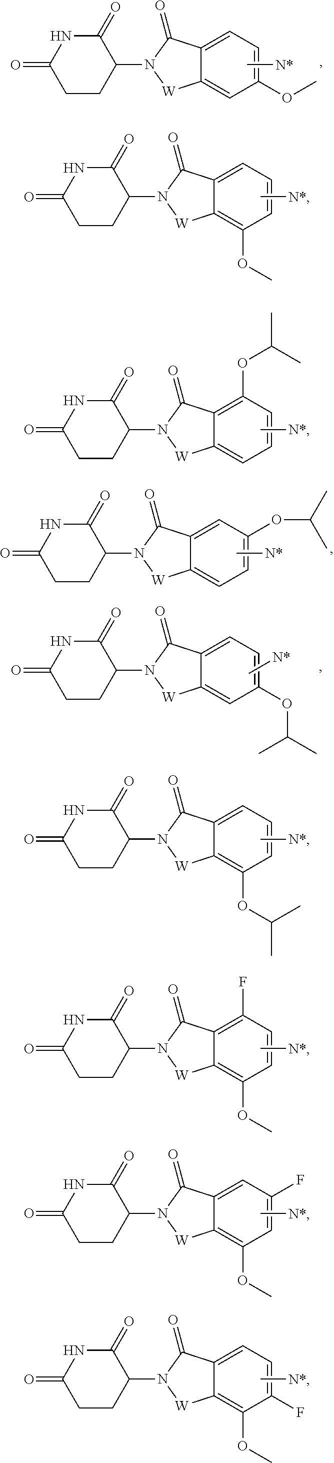

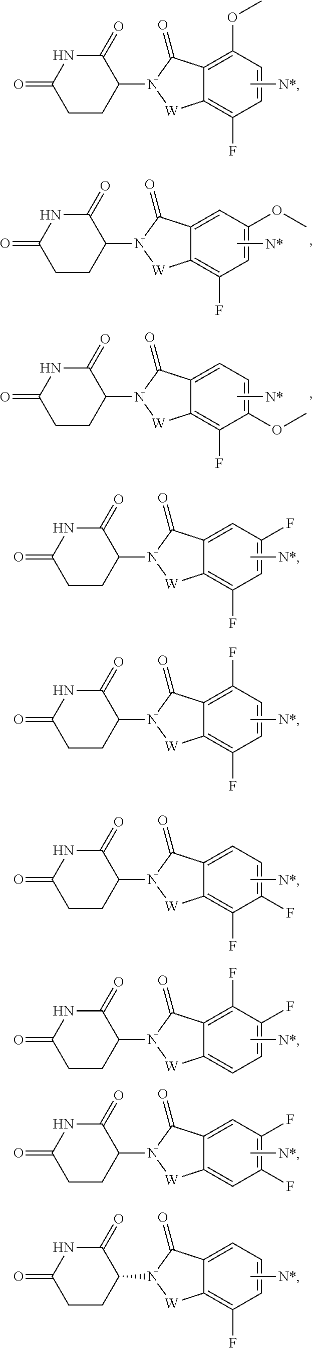

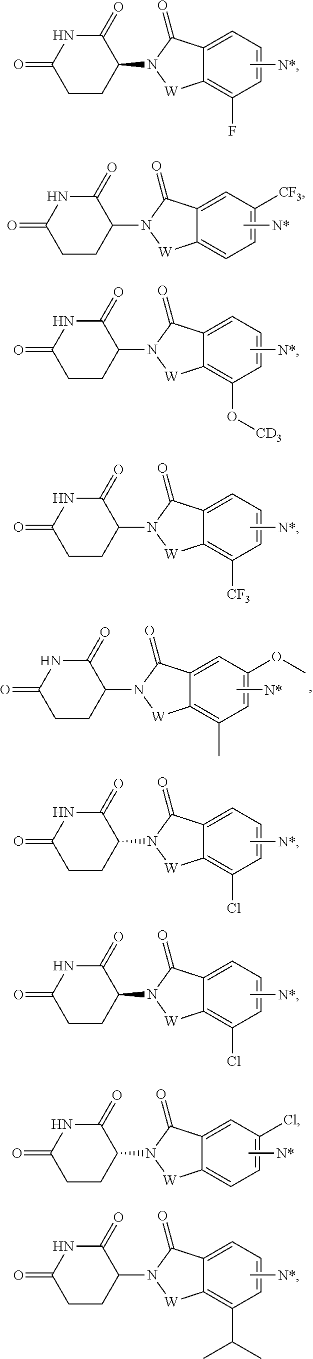

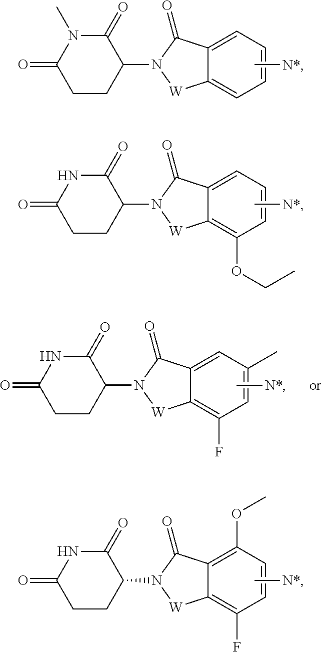

wherein:
   [symbol] of the CLM indicates the point of attachment with the L; and
   N* is a nitrogen atom that is shared with the chemical linker group;
(b) a PTM that is:

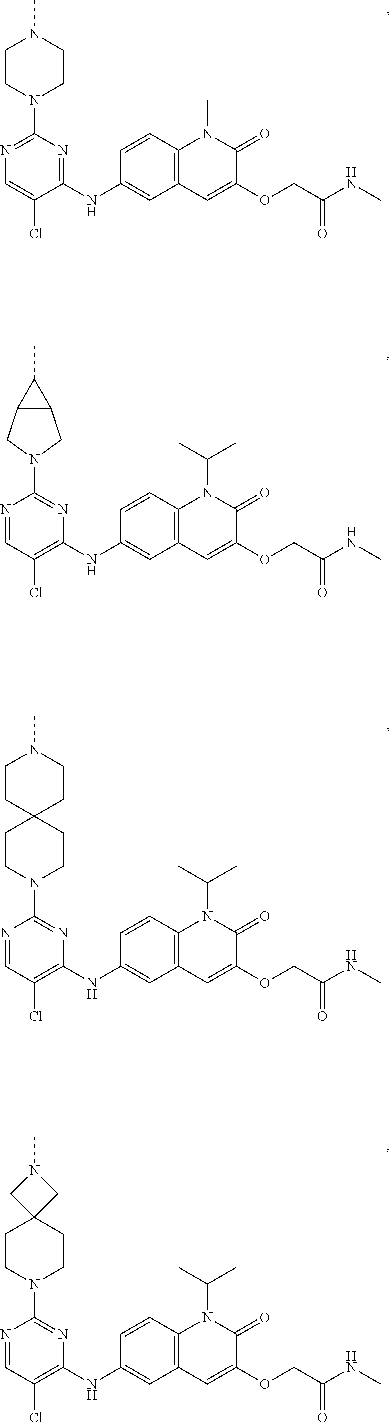

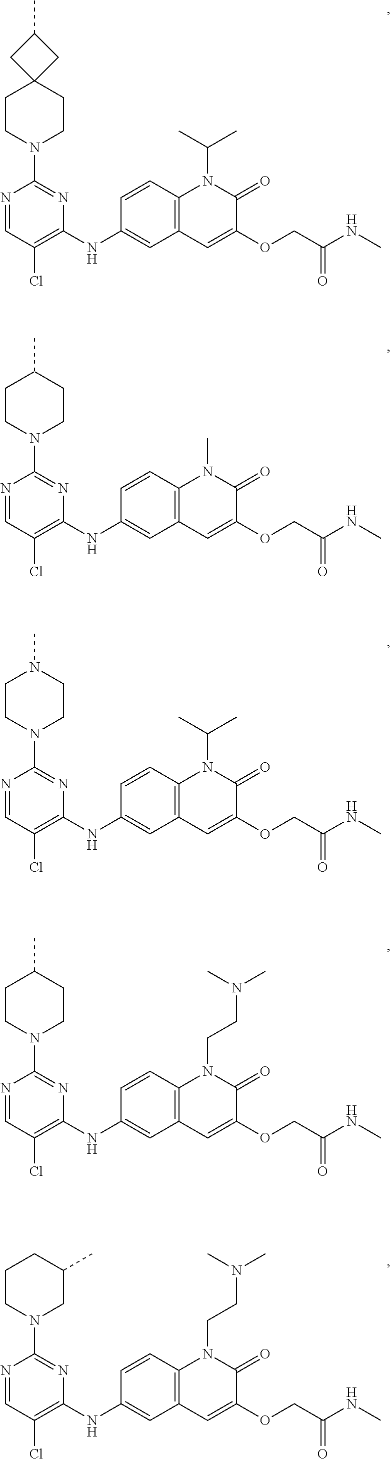

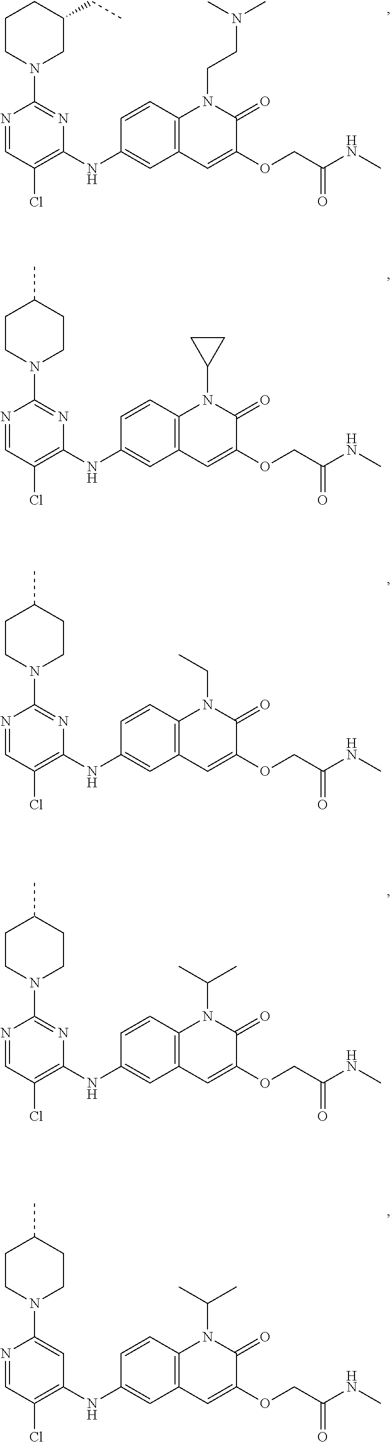

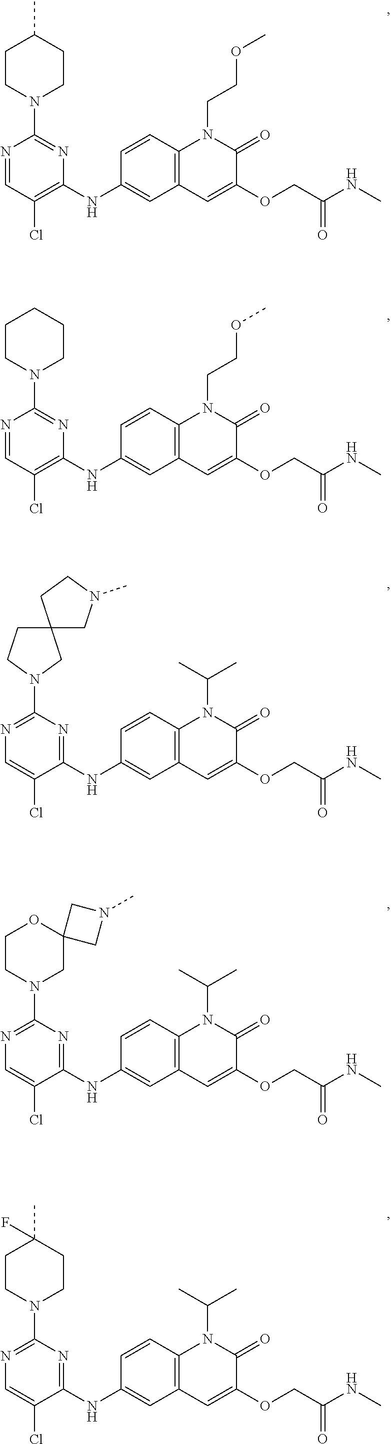

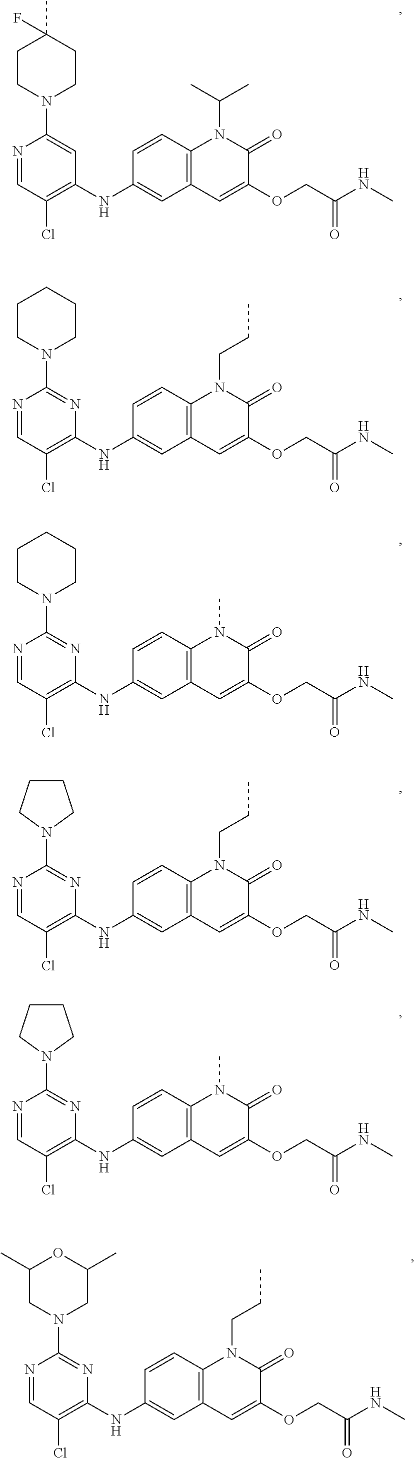

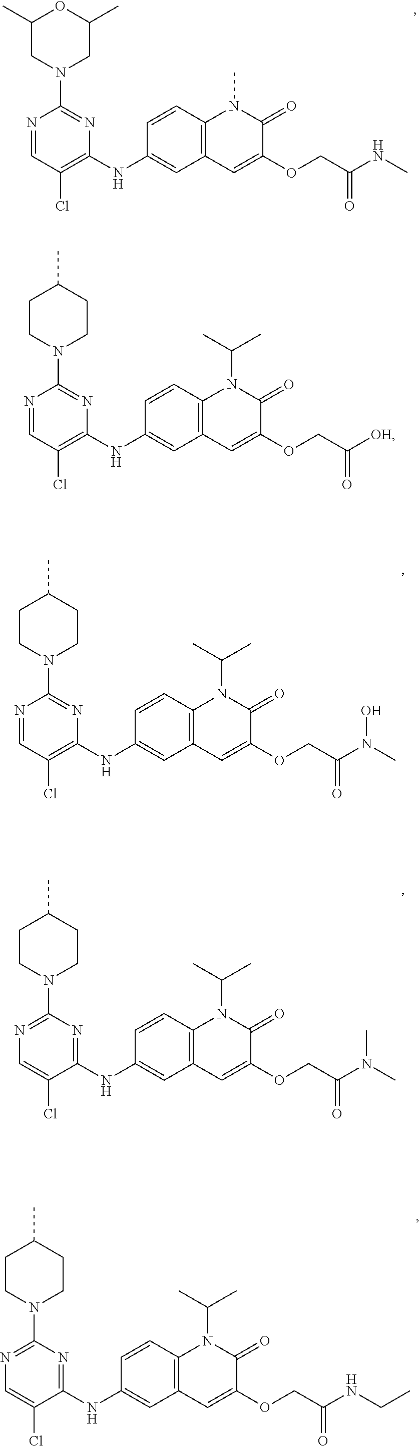

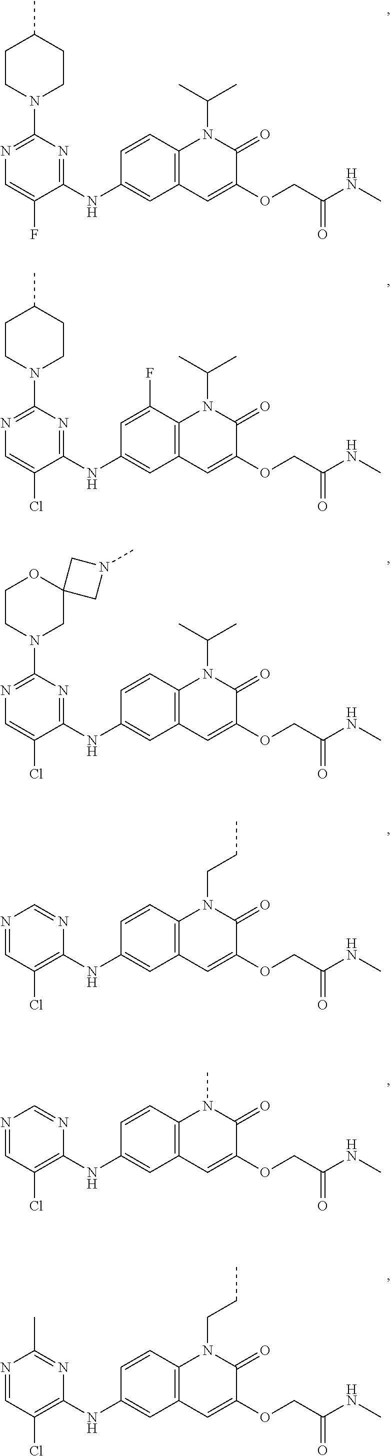

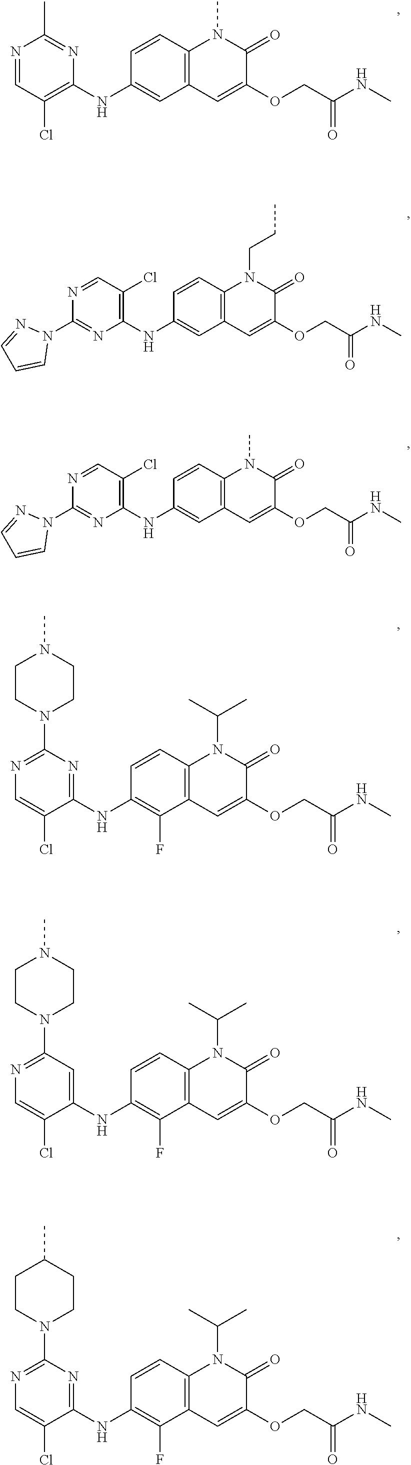

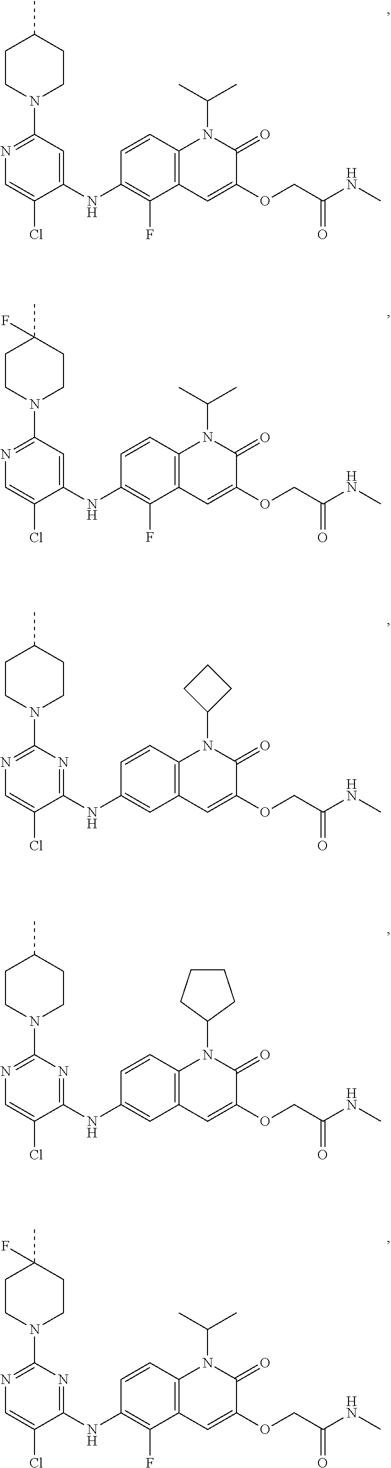

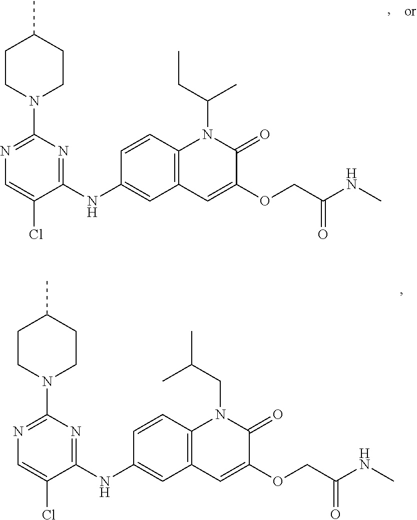

wherein [symbol] of the PTM indicates the point of attachment with the L;
(c) a L that is selected from:

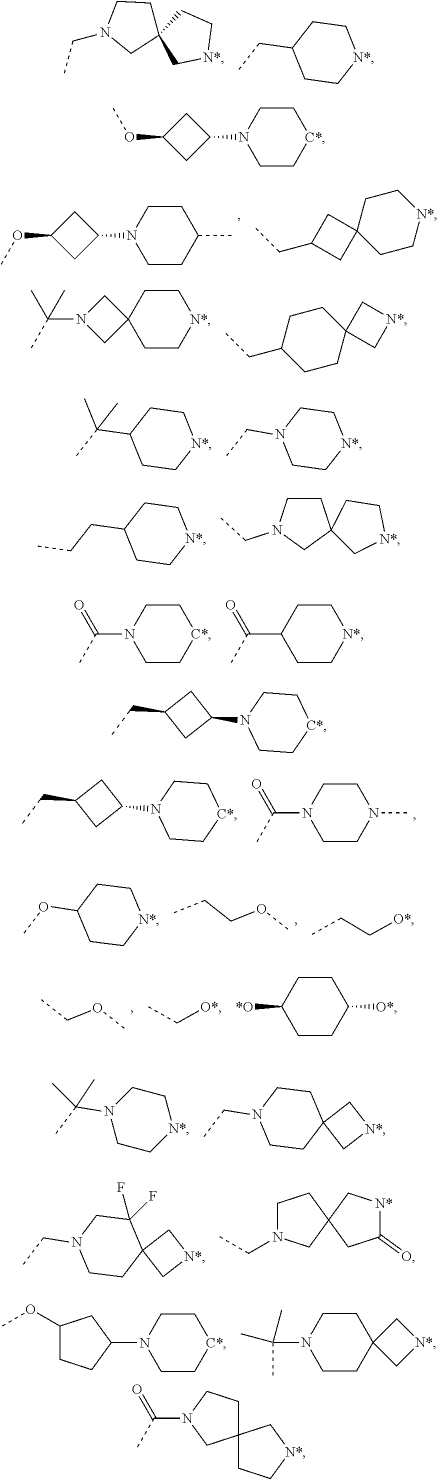

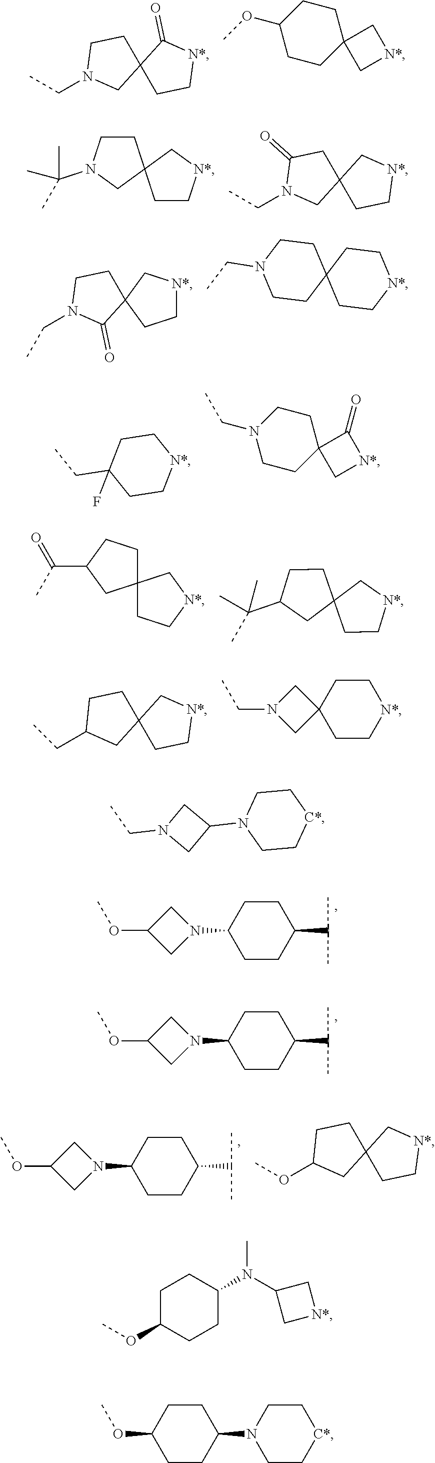

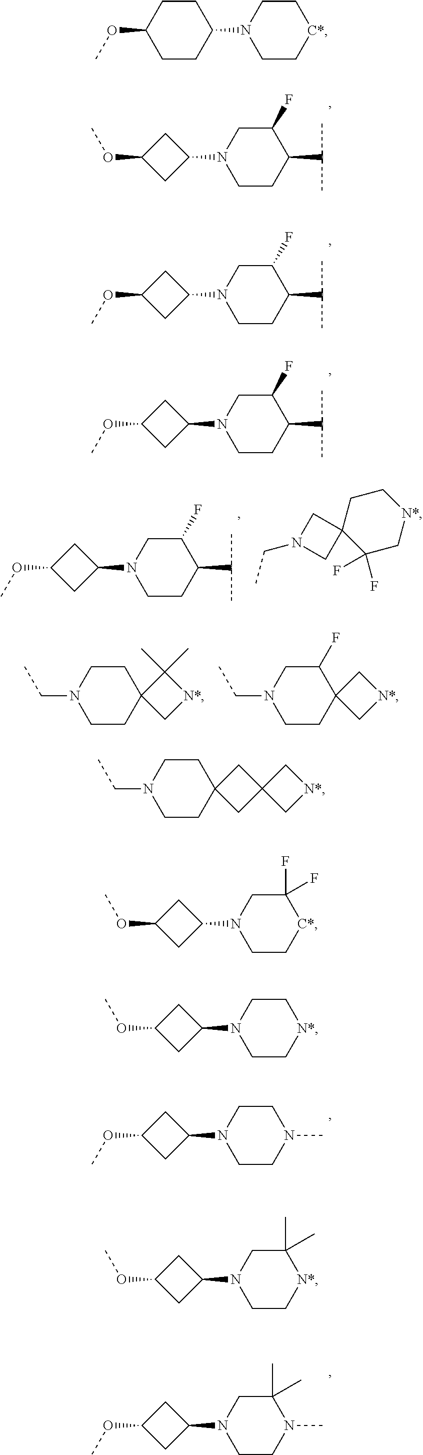

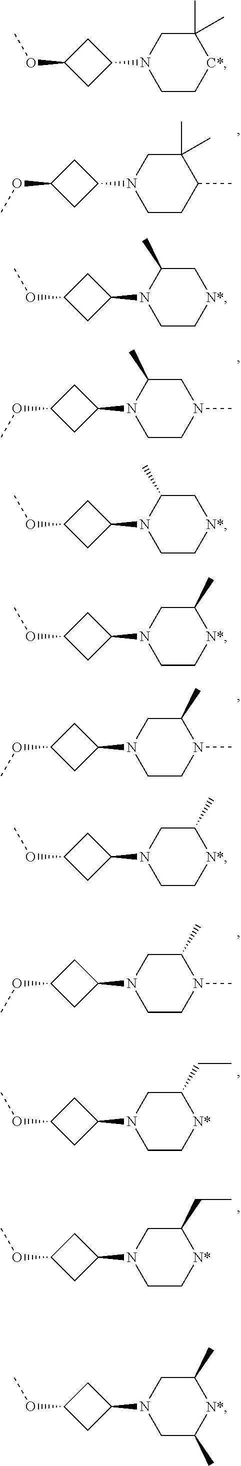

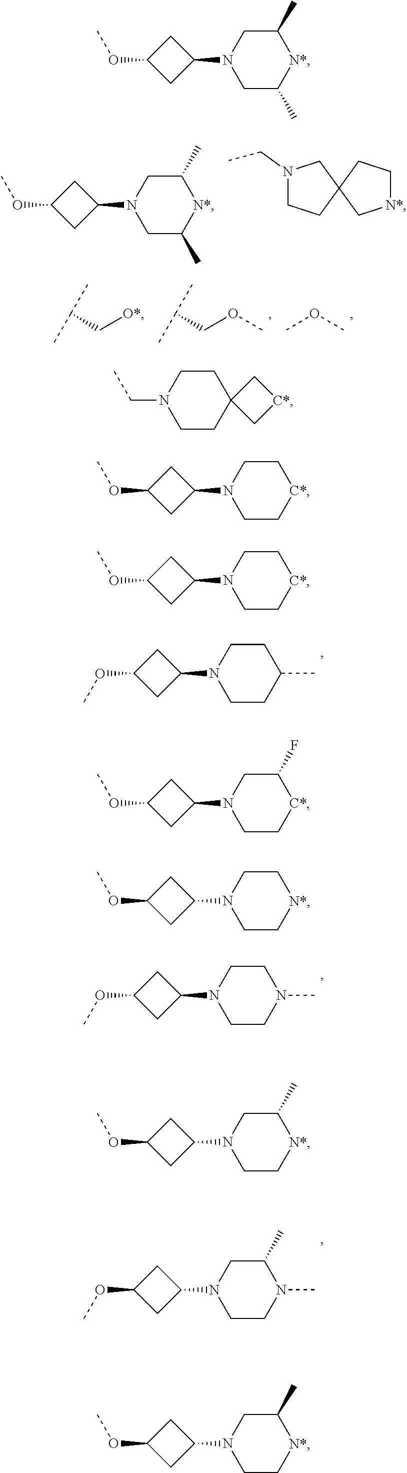

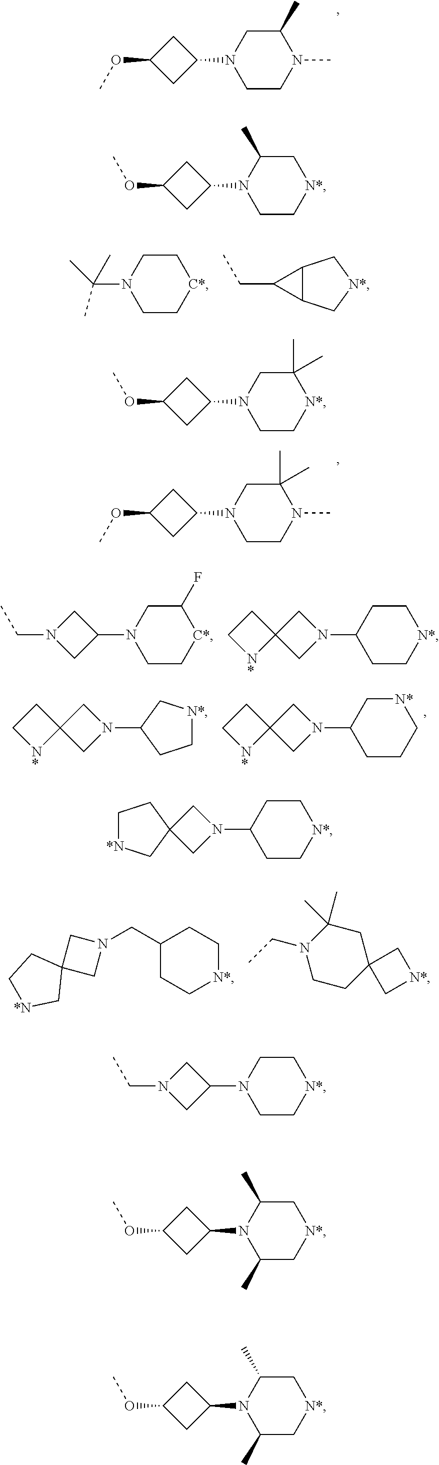

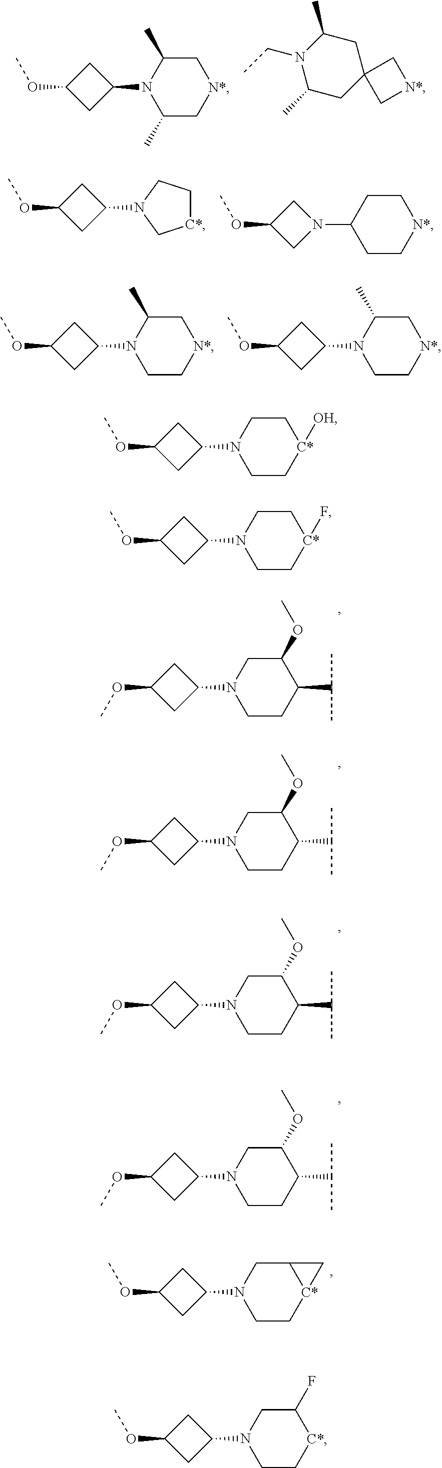

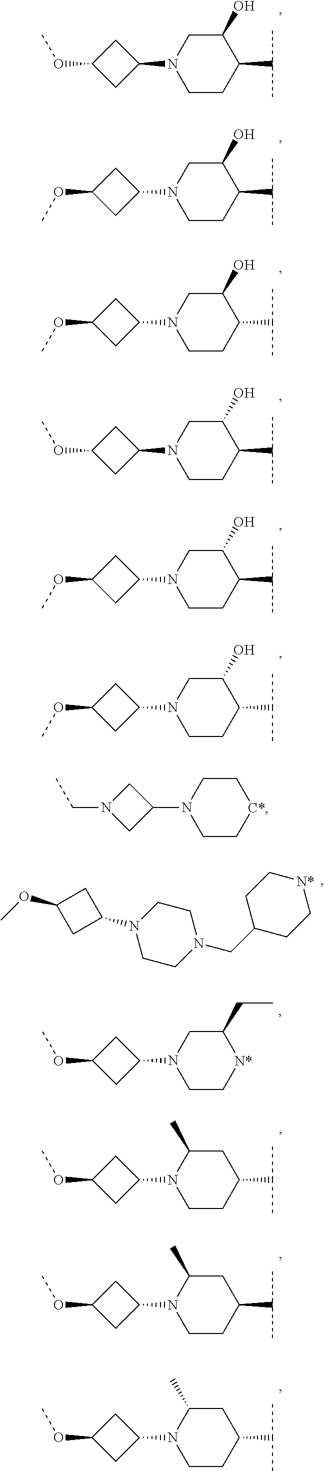

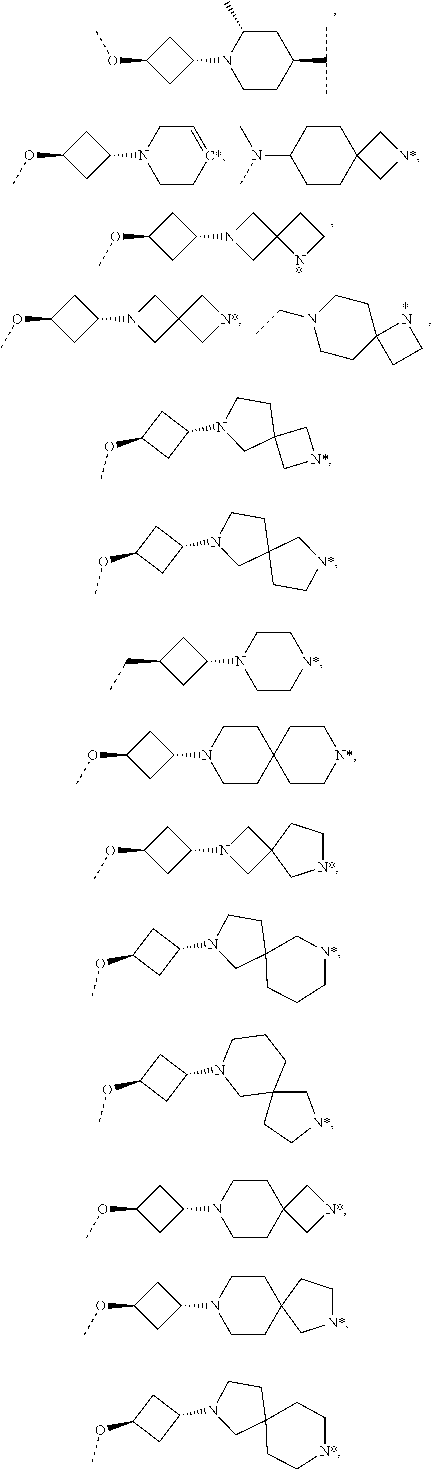

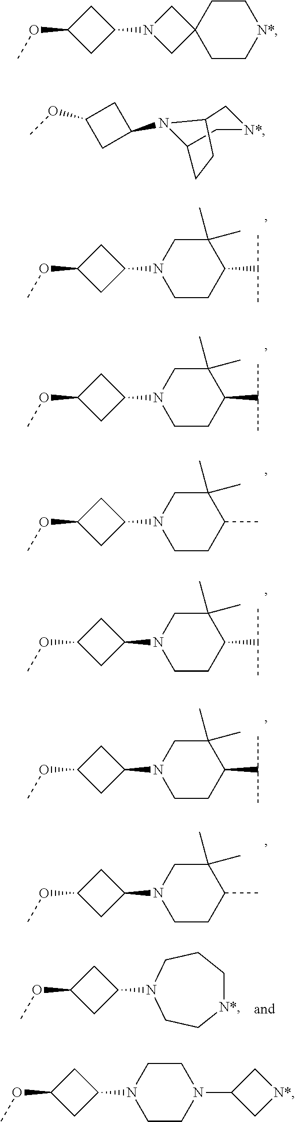

wherein * indicates an atom (e.g., a carbon, nitrogen, or oxygen) that is covalently linked to the CLM or PTM, or that is shared with the CLM or PTM, and each of [symbol] , [symbol] , [symbol] , [symbol] , [symbol] , and [symbol] indicates the point of attachment with the CLM or the PTM; or
(d) any combination of (a), (b), or (c).

In some embodiments, the compound of the disclosure is selected from the list of compounds in Table 1 or a pharmaceutically acceptable salt thereof:

TABLE 1
Compounds of the present disclosure

| Com- | | |
|---|---|---|
| pound | | |
| Num- | | |
| ber | Structure | IUPAC Name |
| 1 | 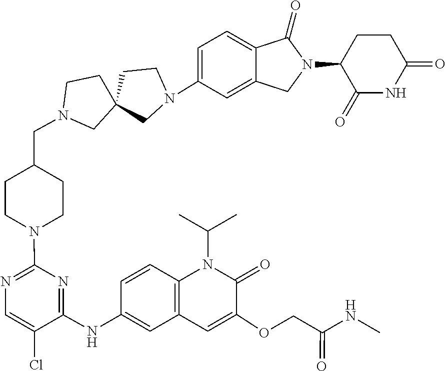 [SMILES: CNC(=O)COc1cc2cc(Nc3nc(N4CCC(CN5CC[C@]6(CCN(c7ccc8c(c7)CN([C@H]7CCC(=O)NC7=O)C8=O)C6)C5)CC4)ncc3Cl)ccc2n(C(C)C)c1=O] | 2-[(6-{[5-chloro-2-(4-{[(5S)-7-{2-[(3S)- 2,6-dioxopiperidin-3-yl]-1-oxo-2,3- dihydro-1H-isoindol-5-yl}-2,7- diazaspiro[4.4]nonan-2- yl]methyl}piperidin-1-yl)pyrimidin-4- yl]amino}-2-oxo-1-(propan-2-yl)-1,2- dihydroquinazolin-3-yl)oxy]-N- methylacetamide |
| 2 | 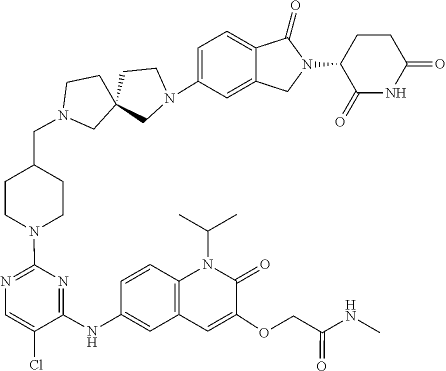 [SMILES: CNC(=O)COc1cc2cc(Nc3nc(N4CCC(CN5CC[C@]6(CCN(c7ccc8c(c7)CN([C@@H]7CCC(=O)NC7=O)C8=O)C6)C5)CC4)ncc3Cl)ccc2n(C(C)C)c1=O] | 2-[(6-{[5-chloro-2-(4-{[(5S)-7-{2-[(3R)- 2,6-dioxopiperidin-3-yl]-1-oxo-2,3- dihydro-1H-isoindol-5-yl}-2,7- diazaspiro[4.4]nonan-2- yl]methyl}piperidin-1-yl)pyrimidin-4- yl]amino}-2-oxo-1-(propan-2-yl)-1,2- dihydroquinolin-3-yl)oxy]-N- methylacetamide |
| 3 | 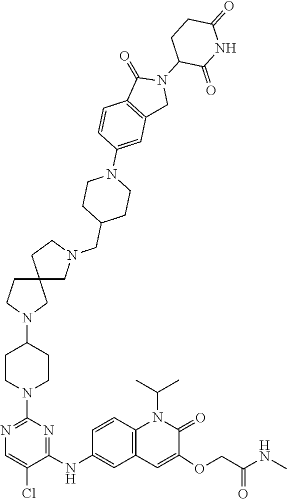 [SMILES: CNC(=O)COc1cc2cc(Nc3nc(N4CCC(N5CCC6(CCN(CC7CCN(c8ccc9c(c8)CN(C8CCC(=O)NC8=O)C9=O)CC7)C6)C5)CC4)ncc3Cl)ccc2n(C(C)C)c1=O] | 2-{[6-({5-chloro-2-[7-({1-[2-(2,6- dioxopiperidin-3-yl)-1-oxo-1,2-dihydro- 1H-isoindol-5-yl]piperidin-4-yl}methyl)- 2,7-diazaspiro[4.4]nonan-2-yl]pyrimidin- 4-yl}amino)-2-oxo-1-(propan-2-yl)-1,2- dihydroquinazolin-3-yl]oxy}-N- methylacetamide |
| 4 | 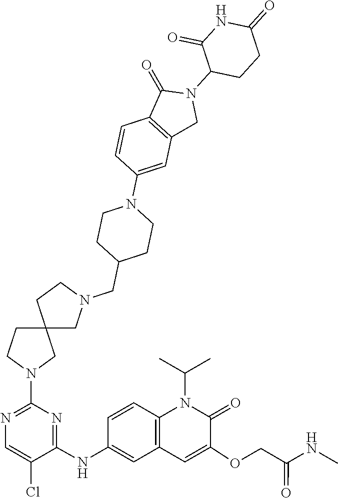 [SMILES: CNC(=O)COc1cc2cc(Nc3nc(N4CCC5(CCN(CC6CCN(c7ccc8c(c7)CN(C7CCC(=O)NC7=O)C8=O)CC6)C5)C4)ncc3Cl)ccc2n(C(C)C)c1=O] | 2-{[6-({5-chloro-2-[7-({1-[2-(2,6- dioxopiperidin-3-yl)-1,3-dioxo-2,3- dihydro-1H-isoindol-5-yl]piperidin-4- yl}methyl)-2,7-diazaspiro[4.4]nonan-2- yl]pyrimidin-4-yl}amino)-2-oxo-1- (propan-2-yl)-1,2-dihydroquinolin-3- yl]oxy}-N-methylacetamide |
| 5 | 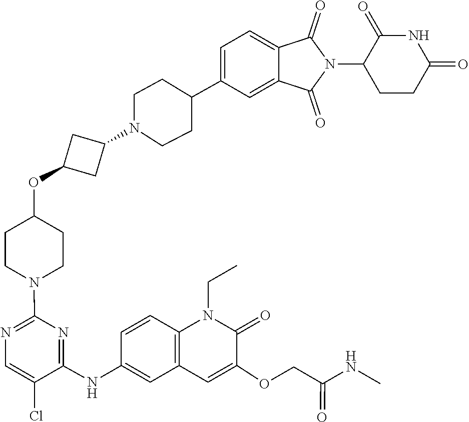 [SMILES: CCn1c(=O)c(OCC(=O)NC)cc2cc(Nc3nc(N4CCC(O[C@H]5C[C@H](N6CCC(c7ccc8c(c7)C(=O)N(C7CCC(=O)NC7=O)C8=O)CC6)C5)CC4)ncc3Cl)ccc21] | 2-({6-[(5-chloro-2-{4-[(1r,3r)-3-{4-[2- (2,6-dioxopiperidin-3-yl)-1,3-dioxo-2,3- dihydro-1H-isoindol-5-yl]piperidin-1- yl}cyclobutoxy]piperidin-1-yl}pyrimidin- 4-yl)amino]-1-ethyl-2-oxo-1,2- dihydroquinolin-3-yl}oxy)-N- methylacetamide |
| 6 | 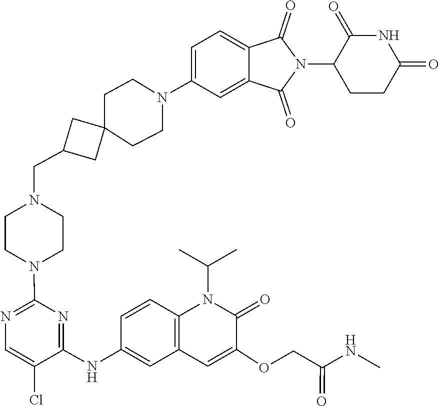 [SMILES: CNC(=O)COc1cc2cc(Nc3nc(N4CCN(CC5CC6(CCN(c7ccc8c(c7)C(=O)N(C7CCC(=O)NC7=O)C8=O)CC6)C5)CC4)ncc3Cl)ccc2n(C(C)C)c1=O] | 2-{[6-({5-chloro-2-[4-({7-[2-(2,6- dioxopiperidin-3-yl)-1-oxo-2,3-dihydro- 1H-isoindol-5-yl]-7-azaspiro[3.5]nonan-2- yl}methyl)piperazin-1-yl]pyrimidin-4- yl}amino)-2-oxo-1-(propan-2-l)-1,2- dihydroquinazolin-3-yl]oxy}-N- methylacetamide |
| 7 | 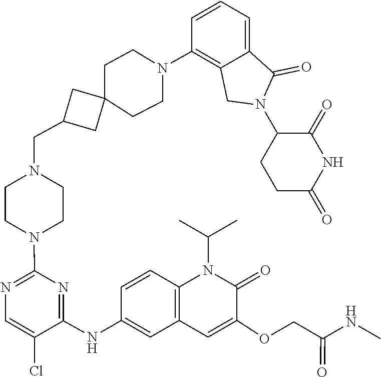 [SMILES: CNC(=O)COc1cc2cc(Nc3nc(N4CCN(CC5CC6(CCN(c7cccc8c7CN(C7CCC(=O)NC7=O)C8=O)CC6)C5)CC4)ncc3Cl)ccc2n(C(C)C)c1=O] | 2-{[6-({5-chloro-2-[4-({7-[2-(2,6- dioxopiperidin-3-yl)-1-oxo-2,3-dihydro- 1H-isoindol-4-yl]-7-azaspiro[3.5]nonan-2- yl}methyl)piperazin-1-yl]pyrimidin-4- yl}amino)-2-oxo-1-(propan-2-yl)-1,2- dihydroquinolin-3-yl]oxy}-N- methylacetamide |
| 8 | 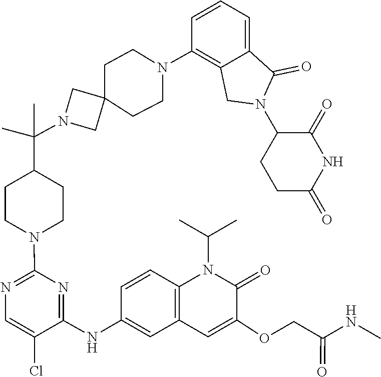 [SMILES: CNC(=O)COc1cc2cc(Nc3nc(N4CCC(C(C)(C)N5CC6(CCN(c7cccc8c7CN(C7CCC(=O)NC7=O)C8=O)CC6)C5)CC4)ncc3Cl)ccc2n(C(C)C)c1=O] | 2-{[6-({5-chloro-2-[4-(2-{7-[2-(2,6- dioxopiperidin-3-yl)-1-oxo-2,3-dihydro- 1H-isoindol-4-yl]-2,7- diazaspiro[3.5]nonan-2-yl}propan-2- yl)piperidin-1-yl]pyrimidin-4-yl}amino)- 2-oxo-1-(propan-2-yl)-1,2- dihydroquinolin-3-yl]oxy}-N- methylacetamide |
| 9 | 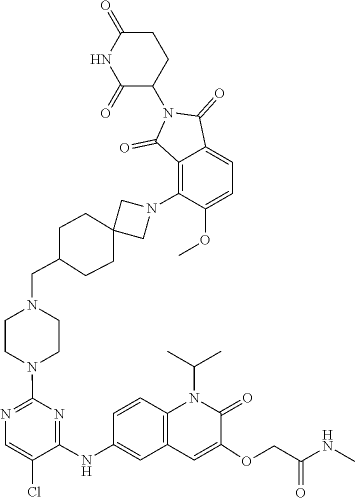 [SMILES: CNC(=O)COc1cc2cc(Nc3nc(N4CCN(CC5CCC6(CC5)CN(c5c(OC)ccc7c5C(=O)N(C5CCC(=O)NC5=O)C7=O)C6)CC4)ncc3Cl)ccc2n(C(C)C)c1=O] | 2-{[6-({5-chloro-2-[4-({2-[2-(2,6- dioxopiperidin-3-yl)-5-methoxy-1,3- dioxo-2,3-dihydro-1H-isoindol-5-yl]-2- azaspiro[3.5]nonan-7- yl}methyl)piperazin-1-yl]pyrimidin-4- yl}amino)-2-oxo-1-(propan-2-yl)-1,2- dihydroquinolin-3-yl]oxy}-N- methylacetamide |
| 10 | 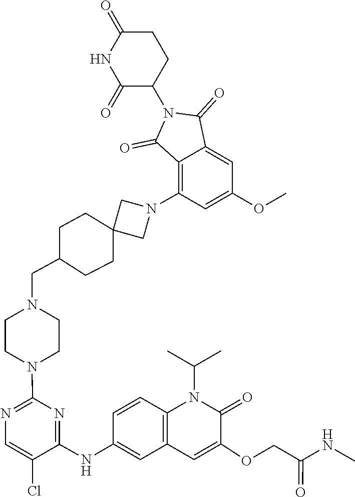 [SMILES: CNC(=O)COc1cc2cc(Nc3nc(N4CCN(CC5CCC6(CC5)CN(c5cc(OC)cc7c5C(=O)N(C5CCC(=O)NC5=O)C7=O)C6)CC4)ncc3Cl)ccc2n(C(C)C)c1=O] | 2-{[6-({5-chloro-2-[4-({2-[2-(2,6- dioxopiperidin-3-yl)-6-methoxy-1,3- dioxo-2,3-dihydro-1H-isoindol-4-yl]-2- azaspiro[3.5]nonan-7- yl}methyl)piperazin-1-yl]pyrimidin-4- yl}amino)-2-oxo-1-(propan-2-yl)-1,2- dihydroquinolin-3-yl]oxy}-N- methylacetamide |
| 11 | 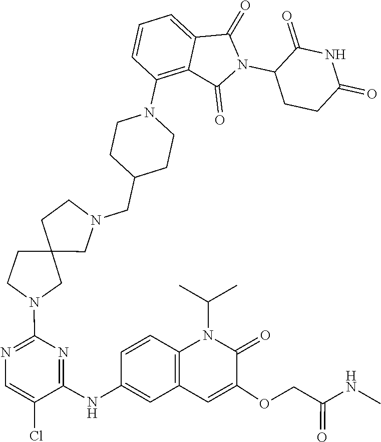 [SMILES: CNC(=O)COc1cc2cc(Nc3nc(N4CCC5(CCN(CC6CCN(c7cccc8c7C(=O)N(C7CCC(=O)NC7=O)C8=O)CC6)C5)C4)ncc3Cl)ccc2n(C(C)C)c1=O] | 2-{[6-({5-chloro-2-[7-({1-[2-(2,6- dioxopiperidin-3-yl)-1,3-dioxo-2,3- dihydro-1H-isoindol-4-yl]piperidin-4- yl}methyl)-2,7-diazaspiro[4.4]nonan-2- yl]pyrimidin-4-yl}amino)-2-oxo-1- (propan-2-yl)-1,2-dihydroquinazolin-3- yl]oxy}-N-methylacetamide |
| 12 | 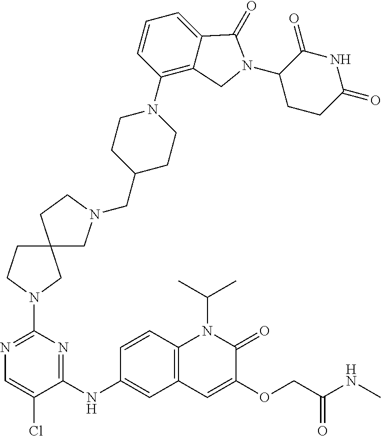 [SMILES: CNC(=O)COc1cc2cc(Nc3nc(N4CCC5(CCN(CC6CCN(c7cccc8c7CN(C7CCC(=O)NC7=O)C8=O)CC6)C5)C4)ncc3Cl)ccc2n(C(C)C)c1=O] | 2-{[6-({5-chloro-2-[7-({1-[2-(2,6- dioxopiperidin-3-yl)-1-oxo-2,3-dihydro- 1H-isoindol-4-yl]piperidin-4-yl}methyl)- 2,7-diazaspiro[4.4]nonan-2-yl]pyrimidin- 4-yl}amino)-2-oxo-1-(propan-2-yl)-1,2- dihydroquinazolin-3-yl]oxy}-N- methylacetamide |
| 13 | 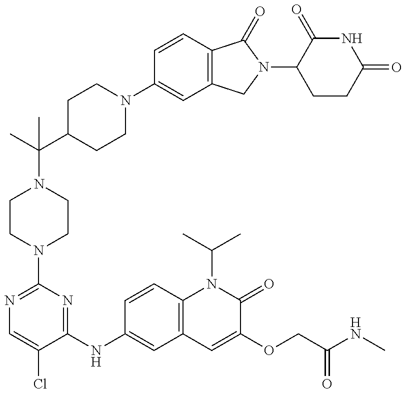 [SMILES: CNC(=O)COc1cc2cc(Nc3nc(N4CCN(C(C)(C)C5CCN(c6ccc7c(c6)CN(C6CCC(=O)NC6=O)C7=O)CC5)CC4)ncc3Cl)ccc2n(C(C)C)c1=O] | 2-{[6-({5-chloro-2-[4-(2-{1-[2-(2,6- dioxopiperidin-3-yl)-1-oxo-2,3-dihydro- 1H-isoindol-5-yl]piperidin-4-yl}propan-2- yl)piperaizn-1-yl]pyrimidin-4-yl}amino)- 2-oxo-1-(propan-2-yl)-1,2- dihydroquinazolin-3-yl]oxy}-N- methylacetamide |
| 14 | 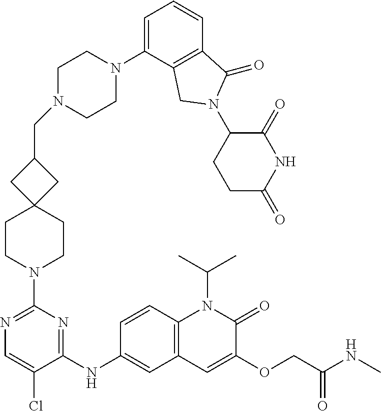 [SMILES: CNC(=O)COc1cc2cc(Nc3nc(N4CCC5(CC4)CC(CN4CCN(c6cccc7c6CN(C6CCC(=O)NC6=O)C7=O)CC4)C5)ncc3Cl)ccc2n(C(C)C)c1=O] | 2-{[6-({5-chloro-2-[2-({4-[2-(2,6- dioxopiperidin-3-yl)-1-oxo-2,3-dihydro- 1H-isoindol-4-yl]piperazin-1-yl}methyl)- 7-azaspiro[3.5]nonan-7-yl]pyrimidin-4- yl}amino)-2-oxo-1-(propan-2-yl)-1,2- dihydroquinazolin-3-yl]oxy}-N- methylacetamide |
| 15 | 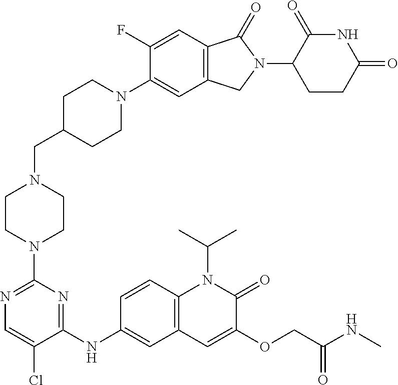 [SMILES: CNC(=O)COc1cc2cc(Nc3nc(N4CCN(CC5CCN(c6cc7c(cc6F)C(=O)N(C6CCC(=O)NC6=O)C7)CC5)CC4)ncc3Cl)ccc2n(C(C)C)c1=O] | 2-{[6-({5-chloro-2-[4-({1-[2-(2,6- dioxopiperidin-3-yl)-6-fluoro-1-oxo-2,3- dihydro-1H-isoindol-5-yl]piperidin-4- yl}methyl)piperazin-1-yl]pyrimidin-4- yl}amino)-2-oxo-1-(propan-2-yl)-1,2- dihydroquinolin-3-yl]oxy}-N- methylacetamide |
| 16 | 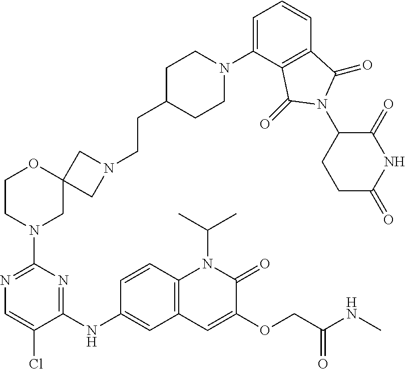 [SMILES: CNC(=O)COc1cc2cc(Nc3nc(N4CCOC5(CN(CCC6CCN(c7cccc8c7C(=O)N(C7CCC(=O)NC7=O)C8=O)CC6)C5)C4)ncc3Cl)ccc2n(C(C)C)c1=O] | |
| 17 | 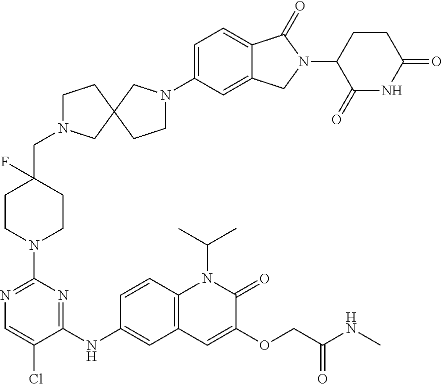 [SMILES: CNC(=O)COc1cc2cc(Nc3nc(N4CCC(F)(CN5CCC6(CCN(c7ccc8c(c7)CN(C7CCC(=O)NC7=O)C8=O)C6)C5)CC4)ncc3Cl)ccc2n(C(C)C)c1=O] | 2-{[6-({5-chloro-2-[4-({7-[2-(2,6- dioxopiperidin-3-yl)-1-oxo-2,3-dihydro- 1H-isoindol-5-yl]-2,7- diazaspiro[4.4]nonan-2-yl}methyl)-4- fluoropiperidin-1-yl]pyrimidin-4- yl}amino)-2-oxo-1-(propan-2-yl)-1,2- dihydroquindolin-3-yl]oxy}-N- methylacetamide |
| 18 | 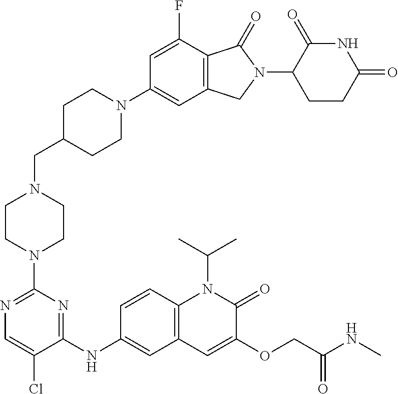 [SMILES: CNC(=O)COc1cc2cc(Nc3nc(N4CCN(CC5CCN(c6cc(F)c7c(c6)CN(C6CCC(=O)NC6=O)C7=O)CC5)CC4)ncc3Cl)ccc2n(C(C)C)c1=O] | 2-{[6-({5-chloro-2-[4-({1-[2-(2,6- dioxopiperidin-3-yl)-7-fluoro-1-oxo-2,3- dihydro-1H-isoindol-5-yl]piperidin-4- yl}methyl)piperazin-1-yl]pyrimidin-4- yl}amino)-2-oxo-1-(propan-2-yl)-1,2- dihydroquinolin-3-yl]oxy}-N- methylacetamide |
| 19 | 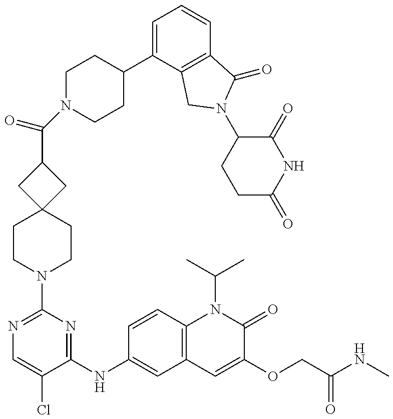 [SMILES: CNC(=O)COc1cc2cc(Nc3nc(N4CCC5(CC4)CC(C(=O)N4CCC(c6cccc7c6CN(C6CCC(=O)NC6=O)C7=O)CC4)C5)ncc3Cl)ccc2n(C(C)C)c1=O] | 2-[(6-{[5-chloro-2-(2-{4-[2-(2,6- dioxopiperidin-3-yl)-1-oxo-2,3-dihydro- 1H-isoindol-4-yl]piperidine-1-carbonyl}- 7-azaspiro[3.5]nonan-7-yl)pyrimidin-4- yl]amino}-2-oxo-1-(propan-2-yl)-1,2- dihydroquinolin-3-yl)oxy]-N- methylacetamide |
| 20 | 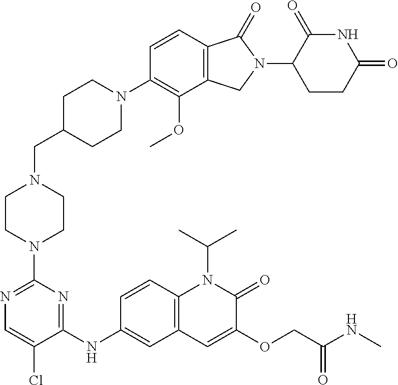 [SMILES: CNC(=O)COc1cc2cc(Nc3nc(N4CCN(CC5CCN(c6ccc7c(c6OC)CN(C6CCC(=O)NC6=O)C7=O)CC5)CC4)ncc3Cl)ccc2n(C(C)C)c1=O] | 2-{[6-({5-chloro-2-[4-({1-[2-(2,6- dioxopiperidin-3-yl)-4-methoxy-1-oxo- 2,3-dihydro-1H-isoindol-5-yl]piperidin-4- yl}methyl)piperazin-1-yl]pyrimidin-4- yl}amino)-2-oxo-1-(propan-2-yl)-1,2- dihydroquinolin-3-l]oxy}-N- methylacetamide |
| 21 | 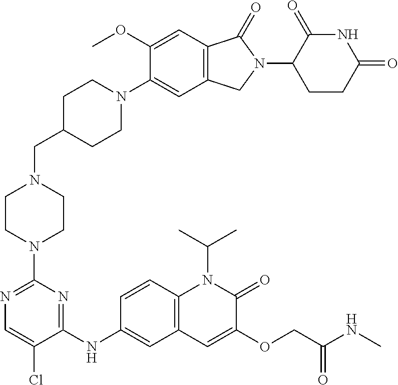 [SMILES: CNC(=O)COc1cc2cc(Nc3nc(N4CCN(CC5CCN(c6cc7c(cc6OC)C(=O)N(C6CCC(=O)NC6=O)C7)CC5)CC4)ncc3Cl)ccc2n(C(C)C)c1=O] | 2-{[6-({5-chloro-2-[4-({1-[2-(2,6- dioxopiperidin-3-yl)-6-methoxy-1-oxo- 2,3-dihydro-1H-isoindol-5-yl]piperidin-4- yl}methyl)piperazin-1-yl]pyrimidin-4- yl}amino)-2-oxo-1-(propan-2-yl)-1,2- dihydroquinolin-3-yl]oxy}-N- methylacetamide |
| 22 | 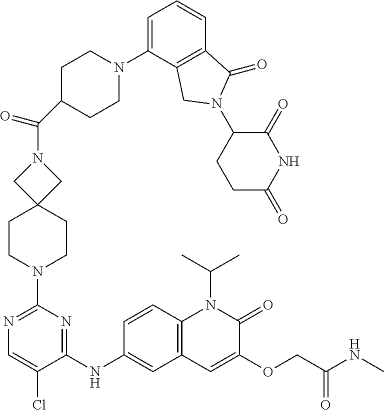 [SMILES: CNC(=O)COc1cc2cc(Nc3nc(N4CCC5(CC4)CN(C(=O)C4CCN(c6cccc7c6CN(C6CCC(=O)NC6=O)C7=O)CC4)C5)ncc3Cl)ccc2n(C(C)C)c1=O] | 2-[(6-{[5-chloro-2-(2-{1-[2-(2,6- dioxopiperidin-3-yl)-1-oxo-2,3-dihydro- 1H-isoindol-4-yl]piperidine-4-carbonyl}- 2,7-diazaspiro[3.5]nonan-7-yl)pyrimidin- 4-yl]amino}-2-oxo-1-(propan-2-yl)-1,2- dihydroquinolin-3-yl)oxy]-N- methylacetamide |
| 23 | 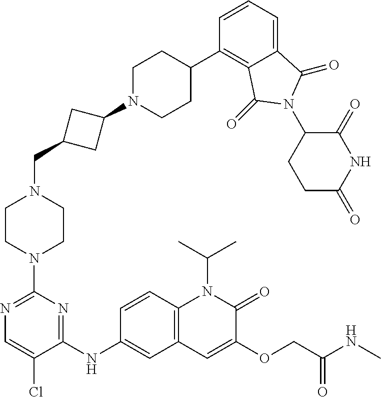 [SMILES: CNC(=O)COc1cc2cc(Nc3nc(N4CCN(C[C@H]5C[C@@H](N6CCC(c7cccc8c7C(=O)N(C7CCC(=O)NC7=O)C8=O)CC6)C5)CC4)ncc3Cl)ccc2n(C(C)C)c1=O] | 2-[(6-{[5-chloro-2-(4-{[(1s,3s)-3-{4-[2- (2,6-dioxopiperidin-3-yl)-1,3-dioxo-2,3- dihydro-1H-isoindol-4-yl]piperidin-1- yl}cyclobutyl]methyl}piperazin-1- yl)pyrimidin-4-yl]amino}-2-oxo-1- (propan-2-yl)-1,2-dihydroquinazolin-3- yl)oxy]-N-methylacetamide |
| 24 | 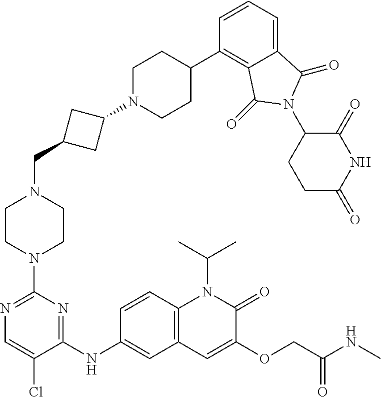 [SMILES: CNC(=O)COc1cc2cc(Nc3nc(N4CCN(C[C@H]5C[C@H](N6CCC(c7cccc8c7C(=O)N(C7CCC(=O)NC7=O)C8=O)CC6)C5)CC4)ncc3Cl)ccc2n(C(C)C)c1=O] | 2-[(6-{[5-chloro-2-(4-{[(1r,3r)-3-{4-[2- (2,6-dioxopiperidin-3-yl)-1,3-dioxo-2,3- dihydro-1H-isoindol-4-yl]piperidin-1- yl}cyclobutyl]methyl}piperazin-1- yl)pyrimidin-4-yl]amino}-2-oxo-1- (propan-2-yl)-1,2-dihydroquinolin-3- yl)oxy]-N-methylacetamide |
| 25 | 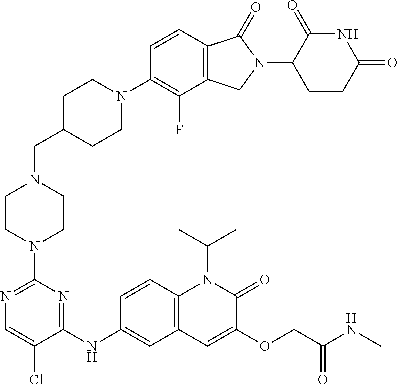 [SMILES: CNC(=O)COc1cc2cc(Nc3nc(N4CCN(CC5CCN(c6ccc7c(c6F)CN(C6CCC(=O)NC6=O)C7=O)CC5)CC4)ncc3Cl)ccc2n(C(C)C)c1=O] | 2-{[6-({5-chloro-2-[4-({1-[2-(2,6- dioxopiperidin-3-yl)-4-fluoro-1-oxo-2,3- dihydro-1H-isoindol-5-yl]piperidin-4- yl}methyl)piperazin-1-yl]pyrimidin-4- yl}amino)-2-oxo-1-(propan-2-yl)-1,2- dihydroquinolin-3-yl]oxy}-N- methylacetamide |
| 26 | 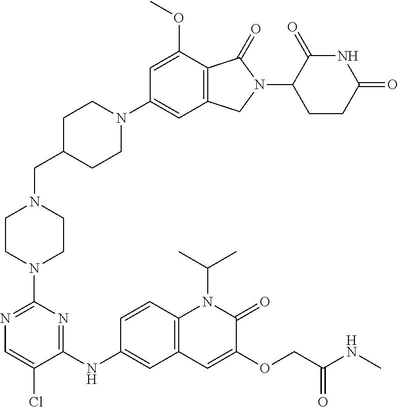 [SMILES: CNC(=O)COc1cc2cc(Nc3nc(N4CCN(CC5CCN(c6cc7c(c(OC)c6)C(=O)N(C6CCC(=O)NC6=O)C7)CC5)CC4)ncc3Cl)ccc2n(C(C)C)c1=O] | 2-{[6-({5-chloro-2-[4-({1-[2-(2,6- dioxopiperidin-3-yl)-7-methoxy-1-oxo- 2,3-dihydro-1H-isoindol-5-yl]piperidin-4- yl}methyl)piperazin-1-yl]pyrimidin-4- yl}amino)-2-oxo-1-(propan-2-yl)-1,2- dihydroquinolin-3-yl]oxy}-N- methylacetamide |
| 27 | 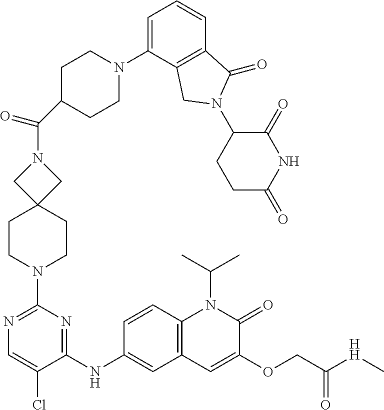 | 2-[(6-{[5-chloro-2-(2-{4-[2-(2,6- dioxopiperidin-3-yl)-1-oxo-2,3-dihydro- 1H-isoindol-4-yl]piperazine-1-carbonyl}- 7-azaspiro[3.5]nonan-7-yl)pyrimidin-4- yl]amino}-2-oxo-1-(propan-2-yl)-1,2- dihydroquinolin-3-yl)oxy]-N- methylacetamide |
| 28 | 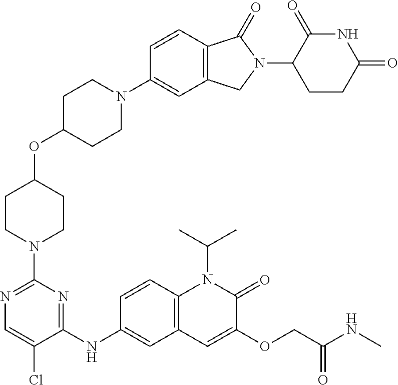 [SMILES: CNC(=O)COc1cc2cc(Nc3nc(N4CCC(OC5CCN(c6ccc7c(c6)CN(C6CCC(=O)NC6=O)C7=O)CC5)CC4)ncc3Cl)ccc2n(C(C)C)c1=O] | 2-{[6-({5-chloro-2-[4-({1-[2-(2,6- dioxopiperidin-3-yl)-1-oxo-2,3-dihydro- 1H-isoindol-5-yl]piperidin-4- yl}oxy)piperidin-1-yl]pyrimidin-4- yl}amino)-2-oxo-1-(propan-2-yl)-1,2- dihydroquinolin-3-yl]oxy}-N- methylacetamide |
| 29 | 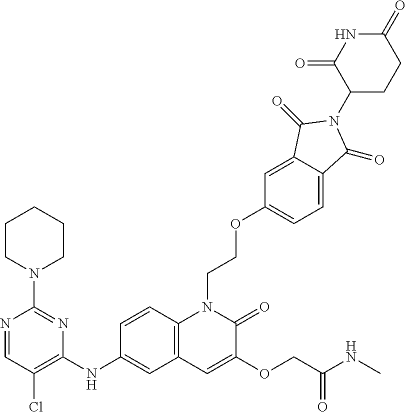 [SMILES: CNC(=O)COc1cc2cc(Nc3nc(N4CCCCC4)ncc3Cl)ccc2n(CCOc2ccc3c(c2)C(=O)N(C2CCC(=O)NC2=O)C3=O)c1=O] | 2-[(6-{[5-chloro-2-(piperidin-1- yl)pyrimidin-4-yl]amino}-1-(2-{[2-(2,6- dioxopiperidin-3-yl)-1,3-dioxo-2,3- dihydro-1H-isoindol-5-yl]oxy}ethyl)-2- oxo-1,2-dihydroquinolin-3-yl)oxy]-N- methyalcetamide |
| 30 | 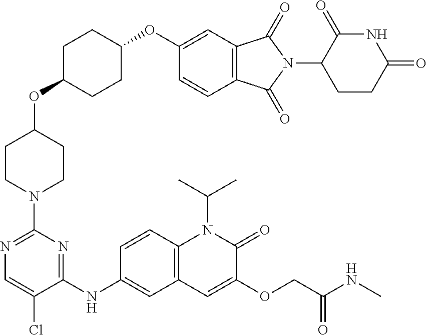 [SMILES: CNC(=O)COc1cc2cc(Nc3nc(N4CCC(O[C@H]5CC[C@H](Oc6ccc7c(c6)C(=O)N(C6CCC(=O)NC6=O)C7=O)CC5)CC4)ncc3Cl)ccc2n(C(C)C)c1=O] | 2-[6-{[5-chloro-2-(4-{[(1r,4r)-4-{[2-(2,6- dioxopiperidin-3-yl)-1,3-dioxo-2,3- dihydro-1H-isoindol-5- yl]oxy}cyclohexyl]oxy}piperidin-1- yl)pyrimidin-4-yl]amino}-2-oxo-1- (propan-2-yl)-1,2-dihydroquinolin-3- yl)oxy]-N-methylacetamide |
| 31 | 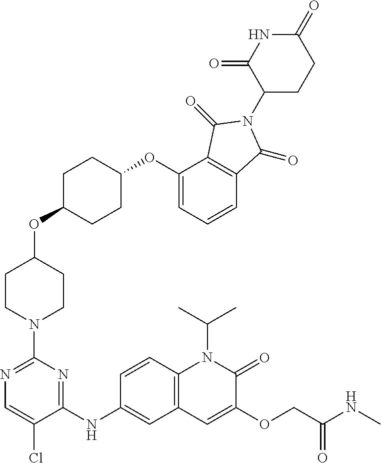 [SMILES: CNC(=O)COc1cc2cc(Nc3nc(N4CCC(O[C@H]5CC[C@H](Oc6cccc7c6C(=O)N(C6CCC(=O)NC6=O)C7=O)CC5)CC4)ncc3Cl)ccc2n(C(C)C)c1=O] | 2-[(6-{[5-choro-2-(4-{[(1r,4r)-4-{[2-(2,6- dioxopiperidin-3-yl)-1,3-dioxo-2,3- dihydro-1H-isoindol-4- yl]oxy}cyclohexyl]oxy}piperidin-1- yl)pyrimidin-4-yl]amino}-2-oxo-1- (propan-2-yl)-1,2-dihydroquinolin-3- yl)oxy]-N-methylacetamide |
| 32 | 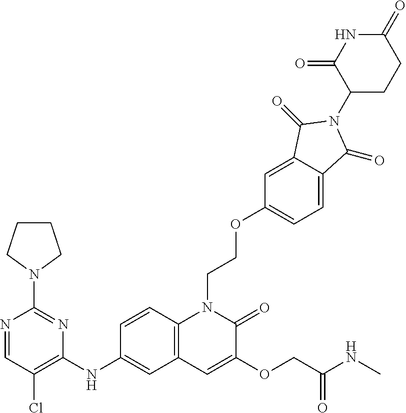 [SMILES: CNC(=O)COc1cc2cc(Nc3nc(N4CCCC4)ncc3Cl)ccc2n(CCOc2ccc3c(c2)C(=O)N(C2CCC(=O)NC2=O)C3=O)c1=O] | 2-[(6-{[5-chloro-2-(pyrrolidin-1- yl)pyrimidin-4-yl]amio}-1-(2-{[2-(2,6- dioxopiperidin-3-yl)-1,3-dioxo-2,3- dihydro-1H-isoinodol-5-yl]oxy}ethyl)-2- oxo-1,2-dihydroquinolin-3-yl)oxy]-N- methylacetamide |
| 33 | 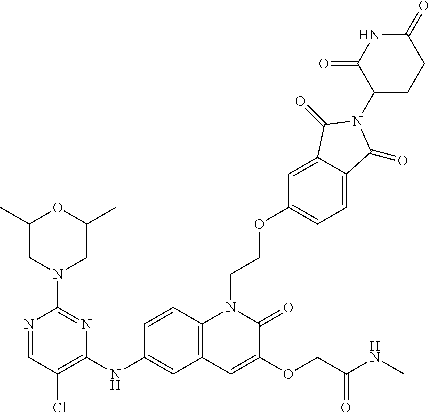 [SMILES: CNC(=O)COc1cc2cc(Nc3nc(N4CC(C)OC(C)C4)ncc3Cl)ccc2n(CCOc2ccc3c(c2)C(=O)N(C2CCC(=O)NC2=O)C3=O)c1=O] | 2-[(6-{[5-chloro-2-(2,6- dimethymorpholin-4-yl)pyrimidin-4- yl]amino}-1-(2-{[2-(2,6-dioxopiperidin-3- yl)-1,3-dioxo-2,3-dihydro-1H-isoindol-5- yl]oxy}ethyl)-2-oxo-1,2-dihydroquinolin- 3-yl)oxy]-N-methylacetamide |
| 34 | 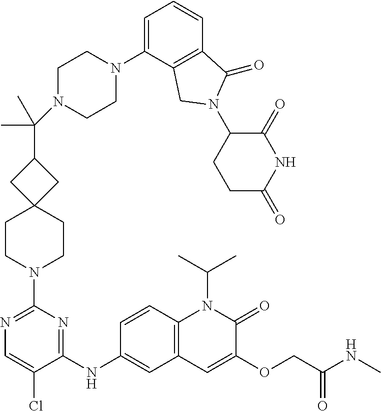 [SMILES: CNC(=O)COc1cc2cc(Nc3nc(N4CCC5(CC4)CC(C(C)(C)N4CCN(c6cccc7c6CN(C6CCC(=O)NC6=O)C7=O)CC4)C5)ncc3Cl)ccc2n(C(C)C)c1=O] | 2-{[6-({5-chloro-2-[2-(2-{4-[2-(2,6- dioxopiperidin-3-yl)-1-oxo-2,3-dihydro- 1H-isoindol-4-yl]piperazin-1-yl}propan- 2-yl)-7-azaspiro[3.5]nonan-7- yl]pyrimidin-4-yl}amino)-2-oxo-1- (propan-2-yl)-1,2-dihydroquinolin-3- yl]oxy}-N-methylacetamide |
| 35 | 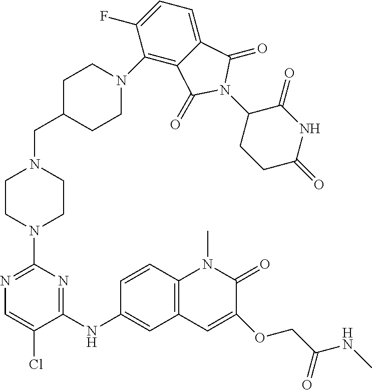 [SMILES: CNC(=O)COc1cc2cc(Nc3nc(N4CCN(CC5CCN(c6c(F)ccc7c6C(=O)N(C6CCC(=O)NC6=O)C7=O)CC5)CC4)ncc3Cl)ccc2n(C)c1=O] | 2-{[6-({5-chloro-2-[4-({1-[2-(2,6- dioxopiperidin-3-yl)-5-fluoro-1,3-dioxo- 2,3-dihydro-1H-isoindol-4-yl]piperidin-4- yl}methyl)piperazin-1-yl]pyrimidin-4- yl}amino)-1-methyl-2-oxo-1,2- dihydroquinolin-3-yl]oxy}-N- methylacetamide |
| 36 | 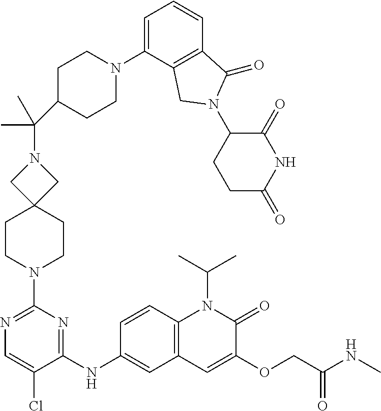 [SMILES: CNC(=O)COc1cc2cc(Nc3nc(N4CCC5(CC4)CN(C(C)(C)C4CCN(c6cccc7c6CN(C6CCC(=O)NC6=O)C7=O)CC4)C5)ncc3Cl)ccc2n(C(C)C)c1=O] | 2-{[6-({5-chloro-2-[2-(2-{1-[2-(2,6- dioxopiperidin-3-yl)-1-oxo-2,3-dihydro- 1H-isoindol-4-yl]piperidin-4-yl}propan-2- yl)-2,7-diazaspiro[3.5]nonan-7- yl]pyrimidin-4-yl}amino)-2-oxo-1- (propan-2-yl)-1,2-dihydroquinolin-3- yl]oxy}-N-methylacetamide |
| 37 | 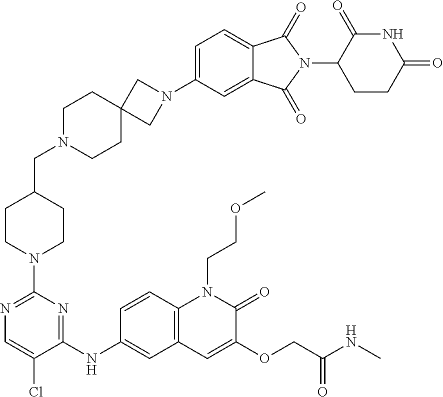 [SMILES: CNC(=O)COc1cc2cc(Nc3nc(N4CCC(CN5CCC6(CC5)CN(c5ccc7c(c5)C(=O)N(C5CCC(=O)NC5=O)C7=O)C6)CC4)ncc3Cl)ccc2n(CCOC)c1=O] | 2-{[6-({5-chloro-2-[4-({2-[2-(2,6- dioxopiperidin-3-yl)-1,3-dioxo-2,3- dihydro-1H-isoindol-5-yl]-2,7- diazaspiro[3.5]nonan-7- yl}methyl)piperidin-1-yl]pyrimidin-4- yl}amino)-1-(2-methoxyethyl)-2-oxo-1,2- dihydroquinolin-3-yl]oxy}-N- methylacetamide |
| 38 | 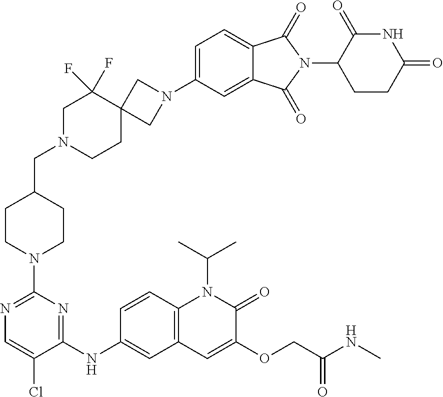 [SMILES: CNC(=O)COc1cc2cc(Nc3nc(N4CCC(CN5CCC6(CN(c7ccc8c(c7)C(=O)N(C7CCC(=O)NC7=O)C8=O)C6)C(F)(F)C5)CC4)ncc3Cl)ccc2n(C(C)C)c1=O] | 2-{[6-({5-chloro-2-[4-({2-[2-(2,6- dioxopiperidin-3-yl)-1,3-dioxo-2,3- dihydro-1H-isoindol-5-yl]-5,5-difluoro- 2,7-diazaspiro[3.5]nonan-7- yl}methyl)piperidin-1-yl]pyrimidin-4- yl}amino)-2-oxo-1-(propan-2-yl)-1,2- dihydroquinolin-3-yl]oxy}-N- methylacetamide |
| 39 | 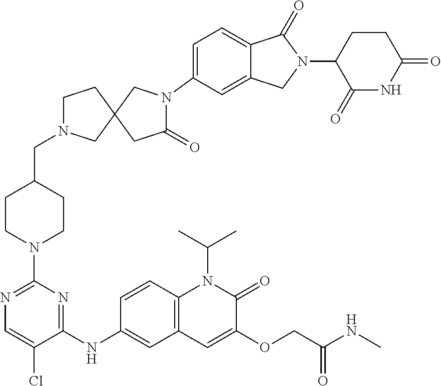 [SMILES: CNC(=O)COc1cc2cc(Nc3nc(N4CCC(CN5CCC6(CC(=O)N(c7ccc8c(c7)CN(C7CCC(=O)NC7=O)C8=O)C6)C5)CC4)ncc3Cl)ccc2n(C(C)C)c1=O] | 2-{[6-({5-chloro-2-[4-({7-[2-(2,6- dioxopiperidin-3-yl)-1-oxo-2,3-dihydro- 1H-isoindol-5-yl]-8-oxo-2,7- diazaspiro[4.4]nonan-2- yl}methyl)piperidin-1-yl]pyrimidin-4- yl}amino)-2-oxo-1-(propan-2-yl)1,2- dihydroquinolin-3-yl]oxy}-N- methylacetamide |
| 40 | 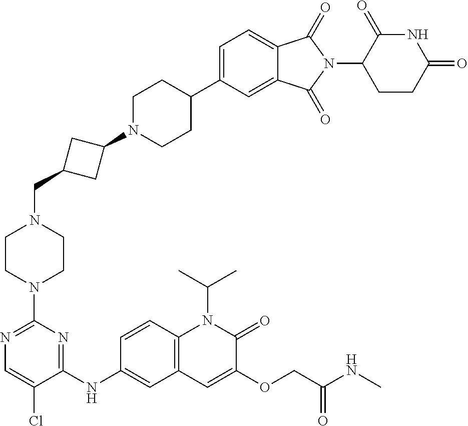 [SMILES: CNC(=O)COc1cc2cc(Nc3nc(N4CCN(C[C@H]5C[C@@H](N6CCC(c7ccc8c(c7)C(=O)N(C7CCC(=O)NC7=O)C8=O)CC6)C5)CC4)ncc3Cl)ccc2n(C(C)C)c1=O] | 2-[6-{[5-chloro-2-(4-{[(1s,3s)-3-{4-[2- (2,6-dioxopiperidin-3-yl)-1,3-dioxo-2,3- dihydro-1H-isoindol-5-yl]piperidin-1- yl}cyclobutyl]methyl}piperazin-1- yl)pyrimidin-4-yl]amino}-2-oxo-1- (propan-2-yl)-1,2-dihydroquinolin-3- yl)oxy]-N-methylacetamide |
| 41 | 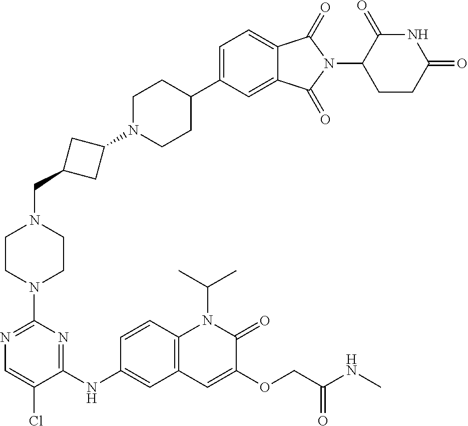 [SMILES: CNC(=O)COc1cc2cc(Nc3nc(N4CCN(C[C@H]5C[C@H](N6CCC(c7ccc8c(c7)C(=O)N(C7CCC(=O)NC7=O)C8=O)CC6)C5)CC4)ncc3Cl)ccc2n(C(C)C)c1=O] | 2-[(6-{[5-chloro-2-(4-{[(1r,3r)-3-{4-[2- (2,6-dioxopiperidin-3-yl)-1,3-dioxo-2,3- dihydro-1H-isoindol-5-yl]piperidin-1- yl}cyclobutyl]methyl}piperazin-1- yl)pyrimidin-4-yl]amino}-2-oxo-1- (propan-2-yl)-1,2-dihydroquinolin-3- yl)oxy]-N-methylacetamide |
| 42 | 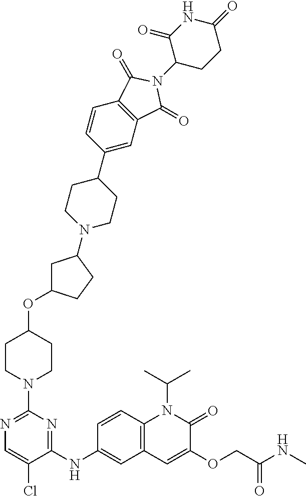 [SMILES: CNC(=O)COc1cc2cc(Nc3nc(N4CCC(OC5CCC(N6CCC(c7ccc8c(c7)C(=O)N(C7CCC(=O)NC7=O)C8=O)CC6)C5)CC4)ncc3Cl)ccc2n(C(C)C)c1=O] | 2-({6-[(5-chloro-2-{4-[(3-{4-[2-(2,6- dioxopiperidin-3-yl)-1,3-dioxo-2,3- dihydro-1H-isoindol-5-yl]piperidin-1- yl}cyclopentyl)oxy]piperidin-1- yl}pyrimidin-4-yl)amino]-2-oxo-1- (propan-2-yl)-1,2-dihydroquinolin-3- yl}oxy)-N-methylacetamide |
| 43 | 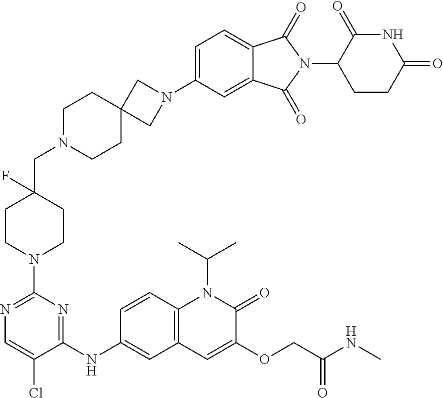 [SMILES: CNC(=O)COc1cc2cc(Nc3nc(N4CCC(F)(CN5CCC6(CC5)CN(c5ccc7c(c5)C(=O)N(C5CCC(=O)NC5=O)C7=O)C6)CC4)ncc3Cl)ccc2n(C(C)C)c1=O] | 2-{[6-({5-chloro-2-[4-({2-[2-(2,6- dioxopiperidin-3-yl)-1,3-dioxo-2,3- dihydro-1H-isoindol-5-yl]-2,7- diazaspiro[3.5]nonan-7-yl}methyl)-4- fluoropiperidin-1-yl]pyrimidin-4- yl}amino)-2-oxo-1-(propan-2-yl)-1,2- dihydroquinolin-3-yl]oxy}-N- methylacetamide |
| 44 | 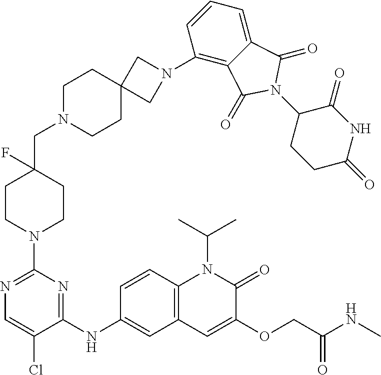 [SMILES: CNC(=O)COc1cc2cc(Nc3nc(N4CCC(F)(CN5CCC6(CC5)CN(c5cccc7c5C(=O)N(C5CCC(=O)NC5=O)C7=O)C6)CC4)ncc3Cl)ccc2n(C(C)C)c1=O] | 2-{[6-({5-chloro-2-[4-({2-[2-(2,6- dioxopiperidin-3-yl)-1,3-dioxo-2,3- dihydro-1H-isoindol-4-yl]-2,7- diazaspiro[3.5]nonan-7-yl}methyl)-4- fluoropiperidin-1-yl]pyrimidin-4- yl}amino)-2-oxo-1-(propan-2-yl)-1,2- dihydroquinolin-3-yl]oxy}-N- methylacetamide |
| 45 | 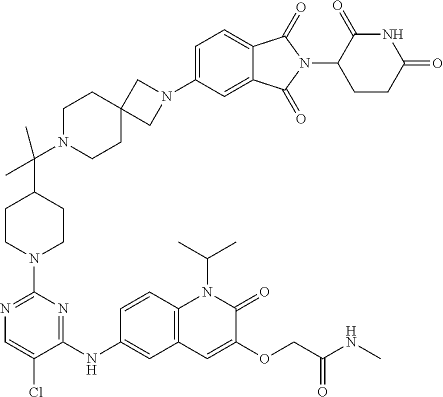 [SMILES: CNC(=O)COc1cc2cc(Nc3nc(N4CCC(C(C)(C)N5CCC6(CC5)CN(c5ccc7c(c5)C(=O)N(C5CCC(=O)NC5=O)C7=O)C6)CC4)ncc3Cl)ccc2n(C(C)C)c1=O] | 2-{[6-({5-chloro-2-[4-(2-{2-[2-(2,6- dioxopiperidin-3-yl)-1,3-dioxo-2,3- dihydro-1H-isoindol-5-yl]-2,7- diazaspior[3.5]nonan-7-yl}propan-2- yl)piperidin-1-yl]pyrimidin-4-yl}amino)- 2-oxo-1-(propan-2-yl)-1,2- dihydroquinolin-3-yl]oxy}-N- methylacetamide |
| 46 | 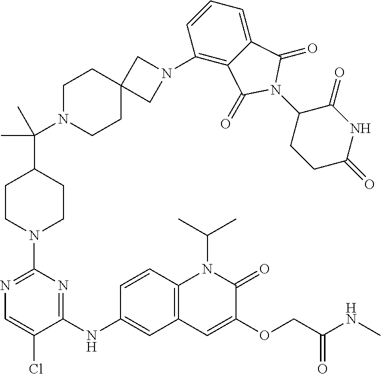 [SMILES: CNC(=O)COc1cc2cc(Nc3nc(N4CCC(C(C)(C)N5CCC6(CC5)CN(c5cccc7c5C(=O)N(C5CCC(=O)NC5=O)C7=O)C6)CC4)ncc3Cl)ccc2n(C(C)C)c1=O] | 2-{[6-({5-chloro-2-[4-(2-{2-[2-(2,6- dioxopiperidin-3-yl)-1,3-dioxo-2,3- dihydro-1H-isoindol-4-yl]-2,7- diazaspiro[3.5]nonan-7-l}propan-2- yl)piperidin-1-yl]pyrimidin-4-yl}amino)- 2-oxo-1-(propan-2-yl)-1,2- dihydroquinolin-3-yl]oxy}-N- methylacetamide |
| 47 | 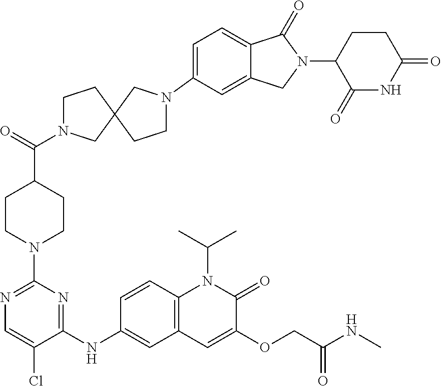 [SMILES: CNC(=O)COc1cc2cc(Nc3nc(N4CCC(C(=O)N5CCC6(CCN(c7ccc8c(c7)CN(C7CCC(=O)NC7=O)C8=O)C6)C5)CC4)ncc3Cl)ccc2n(C(C)C)c1=O] | 2-[(6-{[5-chloro-2-(4-{7-[2-(2,6- dioxopiperidin-3-yl)-1-oxo-2,3-dihydro- 1H-isoindol-5-yl]-2,7- diazaspiro[4.4]nonane-2- carbonyl}piperidin-1-yl)pyrimidin-4- yl]amino}-2-oxo-1-(propan-2-yl)-1,2- dihydroquinolin-3-yl)oxy]-N- methylacetamide |
| 48 | 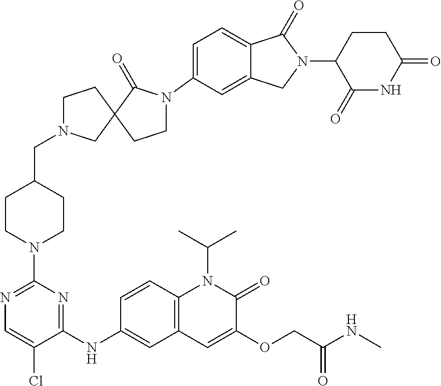 [SMILES: CNC(=O)COc1cc2cc(Nc3nc(N4CCC(CN5CCC6(CCN(c7ccc8c(c7)CN(C7CCC(=O)NC7=O)C8=O)C6=O)C5)CC4)ncc3Cl)ccc2n(C(C)C)c1=O] | 2-{[6-({5-chloro-2-[4-({7-[2-(2,6- dioxopiperidin-3-yl)-1-oxo-2,3-dihydro- 1H-isoindol-5-yl]-6-oxo-2,7- diazaspiro[4.4]nonan-2- yl}methyl)piperidin-1-yl]pyrimidin-4- yl}amino)-2-oxo-1-(propan-2-yl)-1,2- dihydroquinolin-3-yl]oxy}-N- methylacetamide |
| 49 | 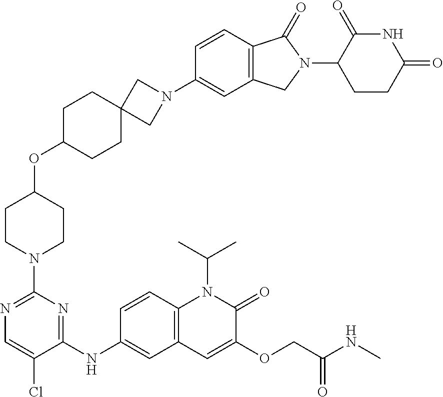 [SMILES: CNC(=O)COc1cc2cc(Nc3nc(N4CCC(OC5CCC6(CC5)CN(c5ccc7c(c5)CN(C5CCC(=O)NC5=O)C7=O)C6)CC4)ncc3Cl)ccc2n(C(C)C)c1=O] | 2-{[6-({5-chloro-2-[4-({2-[2-(2,6- dioxopiperidin-3-yl)-1-oxo-2,3-dihydro- 1H-isoindol-5-yl]-2-azaspiro[3.5]nonan-7- yl}oxy)piperidin-1-yl]pyrimidin-4- yl}amino)-2-oxo-1-(propan-2-yl)-1,2- dihydroquinolin-3-yl]oxy}-N- methylacetamide |
| 50 | 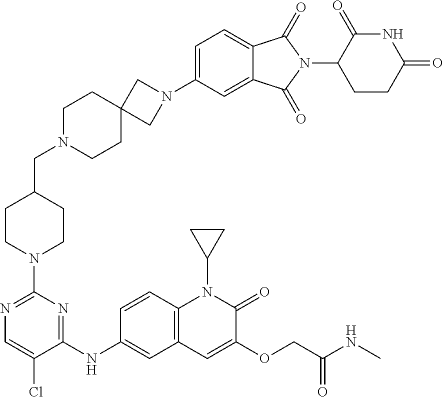 [SMILES: CNC(=O)COc1cc2cc(Nc3nc(N4CCC(CN5CCC6(CC5)CN(c5ccc7c(c5)C(=O)N(C5CCC(=O)NC5=O)C7=O)C6)CC4)ncc3Cl)ccc2n(C2CC2)c1=O] | 2-{[6-({5-chloro-2-[4-({2-[2-(2,6- dioxopiperidin-3-yl)-1,3-dioxo-2,3- dihydro-1H-isoindol-5-yl]-2,7- diazaspiro[3.5]nonan-7- yl}methyl)piperidin-1-yl]pyrimidin-4- yl}amino)-1-cyclopropyl-2-oxo-1,2- dihydroquinolin-3-yl]oxy}-N- methylacetamide |
| 51 | 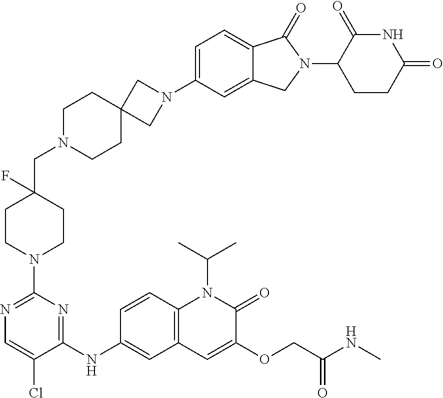 [SMILES: CNC(=O)COc1cc2cc(Nc3nc(N4CCC(F)(CN5CCC6(CC5)CN(c5ccc7c(c5)CN(C5CCC(=O)NC5=O)C7=O)C6)CC4)ncc3Cl)ccc2n(C(C)C)c1=O] | 2-{[6-({5-chloro-2-[4-({2-[2-(2,6- dioxopiperidin-3-yl)-1-oxo-2,3-dihydro- 1H-isoindol-5-yl]-2,7- diazaspiro[3.5]nonan-7-yl}methyl)-4- fluoropiperidin-1-yl]pyrimidin-4- yl}amino)-2-oxo-1-(propan-2-l)-1,2- dihydroquinolin-3-yl]oxy}-N- methylacetamide |
| 52 | 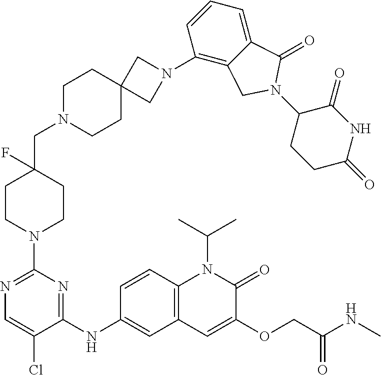 [SMILES: CNC(=O)COc1cc2cc(Nc3nc(N4CCC(F)(CN5CCC6(CC5)CN(c5cccc7c5CN(C5CCC(=O)NC5=O)C7=O)C6)CC4)ncc3Cl)ccc2n(C(C)C)c1=O] | 2-{[6-({5-chloro-2-[4-({2-[2-(2,6- dioxopiperidin-3-yl)-1-oxo-2,3-dihydro- 1H-isoindol-4-yl]-2,7- diazaspiro[3.5]nonan-7-yl}methyl)-4- fluoropiperidin-1-yl]pyrimidin-4- yl}amino)-2-oxo-1-(propan-2-yl)-1,2- dihydroquinolin-3-yl]oxy}-N- methylacetamide |
| 53 | 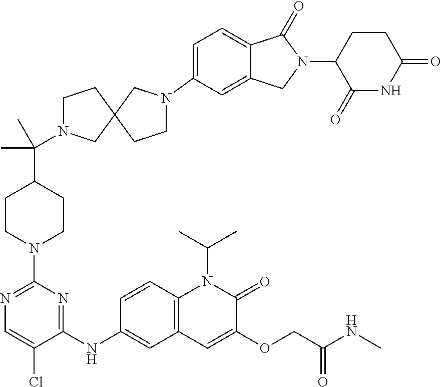 [SMILES: CNC(=O)COc1cc2cc(Nc3nc(N4CCC(C(C)(C)N5CCC6(CCN(c7ccc8c(c7)CN(C7CCC(=O)NC7=O)C8=O)C6)C5)CC4)ncc3Cl)ccc2n(C(C)C)c1=O] | 2-{[6-({5-chloro-2-[4-(2-{7-[2-(2,6- dioxopiperidin-3-yl)-1-oxo-2,3-dihydro- 1H-isoindol-5-yl]-2,7- diazaspiro[4.4]nonan-2-yl}propan-2- yl)piperidin-1-yl]pyrimidin-4-yl}amino)- 2-oxo-1-(propan-2-yl)-1,2- dihydroquinolin-3-yl]oxy}-N- methylacetamide |
| 54 | 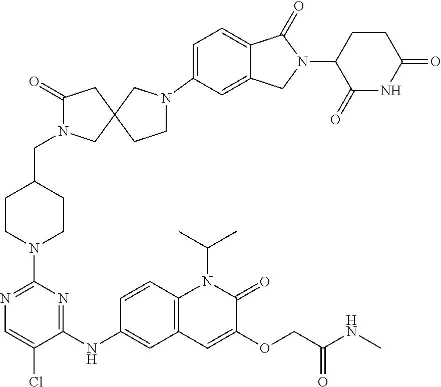 [SMILES: CNC(=O)COc1cc2cc(Nc3nc(N4CCC(CN5CC6(CCN(c7ccc8c(c7)CN(C7CCC(=O)NC7=O)C8=O)C6)CC5=O)CC4)ncc3Cl)ccc2n(C(C)C)c1=O] | 2-{[6-({5-chloro-2-[4-({7-[2-(2,6- dioxopiperidin-3-yl)-1-oxo-2,3-dihydro- 1H-isoindol-5-yl]-3-oxo-2,7- diazaspiro[4.4]nonan-2- yl}methyl)piperidin-1-yl]pyrimidin-4- yl}amino)-2-oxo-1-(propan-2-yl)-1,2- dihydroquinolin-3-yl]oxy}-N- methylacetamide |
| 55 | 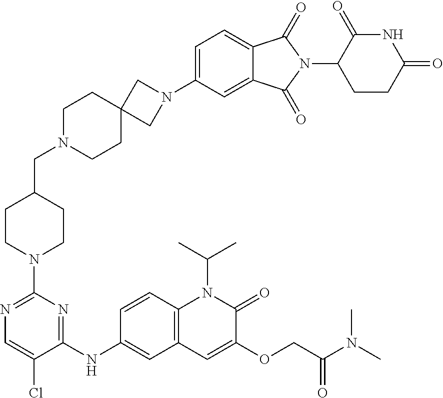 [SMILES: CC(C)n1c(=O)c(OCC(=O)N(C)C)cc2cc(Nc3nc(N4CCC(CN5CCC6(CC5)CN(c5ccc7c(c5)C(=O)N(C5CCC(=O)NC5=O)C7=O)C6)CC4)ncc3Cl)ccc21] | 2-{[6-({5-chloro-2-[4-({2-[2-(2,6- dioxopiperidin-3-yl)-1,3-dioxo-2,3- dihydro-1H-isoindol-5-yl]-2,7- diazaspiro[3.5]nonan-7- yl}methyl)piperidin-1-yl]pyrimidin-4- yl}amino)-2-oxo-1-(propan-2-yl)-1,2- dihydroquinolin-3-yl]oxy}-N,N- dimethylacetamide |
| 56 | 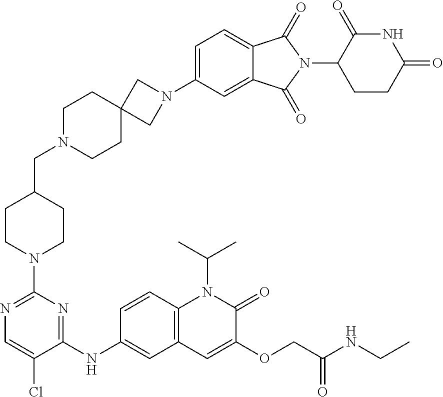 [SMILES: CCNC(=O)COc1cc2cc(Nc3nc(N4CCC(CN5CCC6(CC5)CN(c5ccc7c(c5)C(=O)N(C5CCC(=O)NC5=O)C7=O)C6)CC4)ncc3Cl)ccc2n(C(C)C)c1=O] | 2-{[6-({5-chloro-2-[4-({2-[2-(2,6- dioxopiperidin-3-yl)-1,3-dioxo-2,3- dihydro-1H-isoindol-5-yl]-2,7- diazaspiro[3.5]nonan-7- yl}methyl)piperidin-1-yl]pyrimidin-4- yl}amino)-2-oxo-1-(propan-2-yl)-1,2- dihydroquinolin-3-yl]oxy}-N- ethylacetamide |
| 57 | 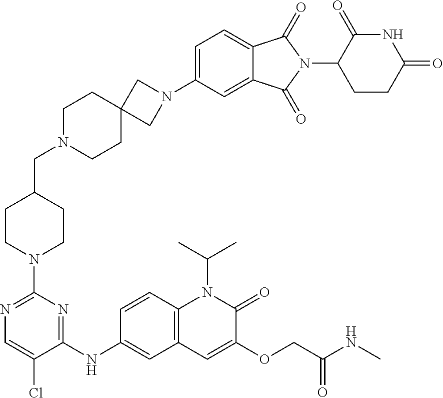 [SMILES: CNC(=O)COc1cc2cc(Nc3nc(N4CCC(CN5CCC6(CC5)CN(c5ccc7c(c5)C(=O)N(C5CCC(=O)NC5=O)C7=O)C6)CC4)ncc3Cl)ccc2n(C(C)C)c1=O] | 2-{[6-({2-[4-({2-[2-(2,6-dioxopiperidin-3- yl)-1,3-dioxo-2,3-dihydro-1H-isoindol-5- yl]-2,7-diazaspiro[3.5]nonan-7- yl}methyl)piperidin-1-yl]-5- fluoropyrimidin-4-yl}amino)-2-oxo-1- (propan-2-yl)-1,2-dihydroquinolin-3- yl]oxy}-N-methylacetamide |
| 58 | 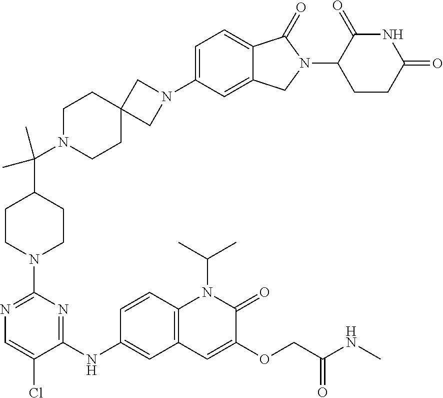 [SMILES: CNC(=O)COc1cc2cc(Nc3nc(N4CCC(C(C)(C)N5CCC6(CC5)CN(c5ccc7c(c5)CN(C5CCC(=O)NC5=O)C7=O)C6)CC4)ncc3Cl)ccc2n(C(C)C)c1=O] | 2-{[6-({5-chloro-2-[4-(2-{2-[2-(2,6- dioxopiperidin-3-yl)-1-oxo-2,3-dihydro- 1H-isoindol-5-yl]-2,7- diazaspiro[3.5]nonan-7-yl}propan-2- yl)piperidin-1-yl]pyrimidin-4-yl}amino)- 2-oxo-1-(propan-2-yl)-1,2- dihydroquinolin-3-yl]oxy}-N- methylacetamide |
| 59 | 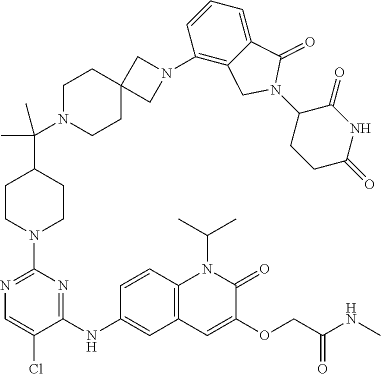 [SMILES: CNC(=O)COc1cc2cc(Nc3nc(N4CCC(C(C)(C)N5CCC6(CC5)CN(c5cccc7c5CN(C5CCC(=O)NC5=O)C7=O)C6)CC4)ncc3Cl)ccc2n(C(C)C)c1=O] | 2-{[6-({5-chloro-2-[4-(2-{2-[2-(2,6- dioxopiperidin-3-yl)-1-oxo-2,3-dihydro- 1H-isoindol-4-yl]-2,7- diazaspiro[3.5]nonan-7-yl}propan-2- yl)piperidin-1-yl]pyrimidin-4-yl}amino)- 2-oxo-1-(propan-2-yl)-1,2- dihydroquinolin-3-yl]oxy}-N- methylacetamide |
| 60 | 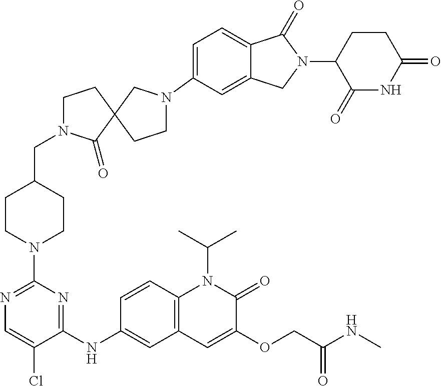 [SMILES: CNC(=O)COc1cc2cc(Nc3nc(N4CCC(CN5CCC6(CCN(c7ccc8c(c7)CN(C7CCC(=O)NC7=O)C8=O)C6)C5=O)CC4)ncc3Cl)ccc2n(C(C)C)c1=O] | 2-{[6-({5-chloro-2-[4-({7-[2-(2,6- dioxopiperidin-3-yl)-1-oxo-2,3-dihydro- 1H-isoindol-5-yl]-1-oxo-2,7- diazaspiro[4.4]nonan-2- yl}methyl)piperidin-1-yl]pyrimidin-4- yl}amino)-2-oxo-1-(propan-2-yl)-1,2- dihydroquinolin-3-yl]oxy}-N- methylacetamide |
| 61 | 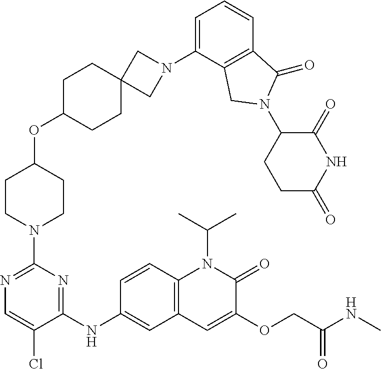 [SMILES: CNC(=O)COc1cc2cc(Nc3nc(N4CCC(OC5CCC6(CC5)CN(c5cccc7c5CN(C5CCC(=O)NC5=O)C7=O)C6)CC4)ncc3Cl)ccc2n(C(C)C)c1=O] | 2-{[6-({5-chloro-2-[4-({2-[2-(2,6- dioxopiperidin-3-yl)-1-oxo-2,3-dihydro- 1H-isoindol-4-yl]-2-azaspiro[3.5]nonan-7- yl}oxy)piperidin-1-yl]pyrimidin-4- yl}amino)-2-oxo-1-(propan-2-yl)-1,2- dihydroquinolin-3-yl]oxy}-N- methylacetamide |
| 62 | 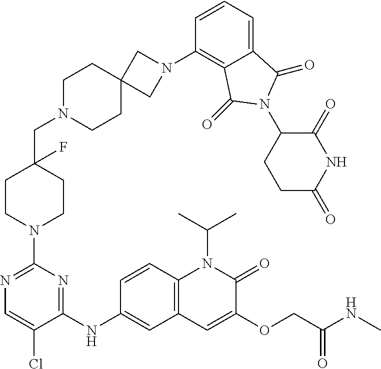 [SMILES: CNC(=O)COc1cc2cc(Nc3nc(N4CCC(F)(CN5CCC6(CC5)CN(c5cccc7c5C(=O)N(C5CCC(=O)NC5=O)C7=O)C6)CC4)ncc3Cl)ccc2n(C(C)C)c1=O] | 2-{[6-({5-chloro-2-[4-({9-[2-(2,6- dioxopiperidin-3-yl)-1,3-dioxo-2,3- dihydro-1H-isoindol-4-yl]-3,9- diazaspiro[5.5]undecan-3-yl}methyl)-4- fluoropiperidin-1-yl]pyrimidin-4- yl}amino)-2-oxo-1-(propan-2-yl)-1,2- dihydroquinolin-3-yl]oxy}-N- methylacetamide |
| 63 | 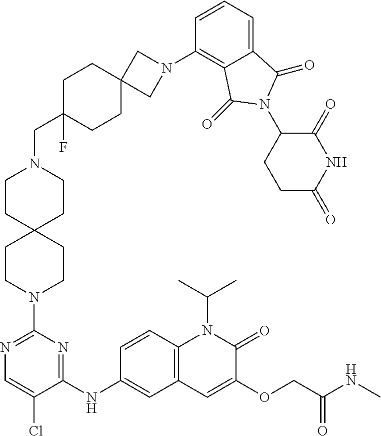 [SMILES: CNC(=O)COc1cc2cc(Nc3nc(N4CCC5(CCN(CC6(F)CCC7(CC6)CN(c6cccc8c6C(=O)N(C6CCC(=O)NC6=O)C8=O)C7)CC5)CC4)ncc3Cl)ccc2n(C(C)C)c1=O] | 2-{[6-({5-chloro-2-[9-({1-[2-(2,6- dioxopiperidin-3-yl)-1,3-dioxo-2,3- dihydro-1H-isoindol-4-yl]-4- fluoropiperidin-4-yl}methyl)-3,9- diazaspiro[5.5]undecan-3-yl]pyrimidin-4- yl}amino)-2-oxo-1-(propan-2-yl)-1,2- dihydroquinolin-3-yl]oxy}-N- methylacetamide |
| 64 | 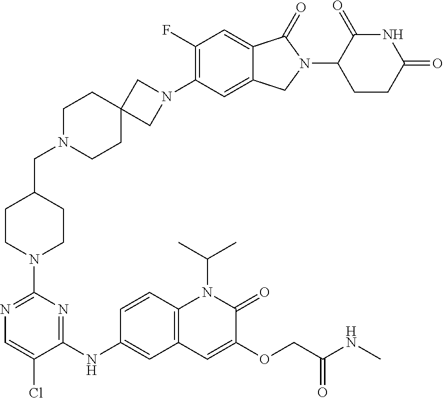 [SMILES: CNC(=O)COc1cc2cc(Nc3nc(N4CCC(CN5CCC6(CC5)CN(c5cc7c(cc5F)C(=O)N(C5CCC(=O)NC5=O)C7)C6)CC4)ncc3Cl)ccc2n(C(C)C)c1=O] | 2-{[6-({5-chloro-2-[4-({2-[2-(2,6- dioxopiperidin-3-yl)-6-fluoro-1-oxo-2,3- dihydro-1H-isoindol-5-yl]-2,7- diazaspiro[3.5]nonan-7- yl}methyl)piperidin-1-yl]pyrimidin-4- yl}amino)-2-oxo-1-(propan-2-yl)-1,2- dihydroquinolin-3-yl]oxy}-N- methylacetamide |
| 65 | 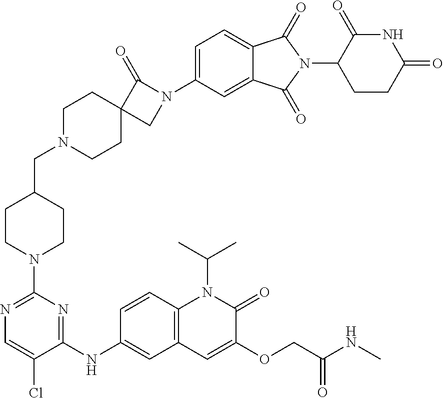 [SMILES: CNC(=O)COc1cc2cc(Nc3nc(N4CCC(CN5CCC6(CC5)CN(c5ccc7c(c5)C(=O)N(C5CCC(=O)NC5=O)C7=O)C6=O)CC4)ncc3Cl)ccc2n(C(C)C)c1=O] | 2-{[6-({5-chloro-2-[4-({2-[2-(2,6- dioxopiperidin-3-yl)-1,3-dioxo-2,3- dihydro-1H-isoindol-5-yl]-1-oxo-2,7- diazaspiro[3.5]nonan-7- yl}methyl)piperidin-1-yl]pyrimidin-4- yl}amino)-2-oxo-1-(propan-2-yl)-1,2- dihydroquinolin-3-yl]oxy}-N- methylacetamide |
| 66 | 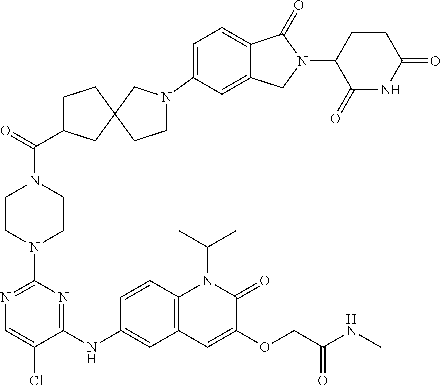 [SMILES: CNC(=O)COc1cc2cc(Nc3nc(N4CCN(C(=O)C5CCC6(CCN(c7ccc8c(c7)CN(C7CCC(=O)NC7=O)C8=O)C6)C5)CC4)ncc3Cl)ccc2n(C(C)C)c1=O] | 2-[(6-{[5-chloro-2-(4-{2-[2-(2,6- dioxopiperidin-3-yl)-1-oxo-2,3-dihydro- 1H-isoindol-5-yl]-2-azaspiro[4.4]nonane- 7-carbonyl}piperazin-1-yl)pyrimidin-4- yl]amino}-2-oxo-1-(propan-2-yl)-1,2- dihydroquinolin-3-yl)oxy]-N- methylacetamide |
| 67 | 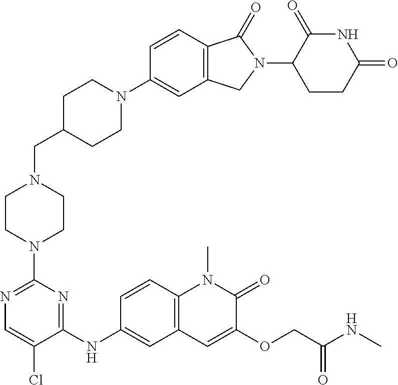 [SMILES: CNC(=O)COc1cc2cc(Nc3nc(N4CCN(CC5CCN(c6ccc7c(c6)CN(C6CCC(=O)NC6=O)C7=O)CC5)CC4)ncc3Cl)ccc2n(C)c1=O] | 2-{[6-({5-chloro-2-[4-({1-[2-(2,6- dioxopiperidin-3-yl)-1-oxo-2,3-dihydro- 1H-isoindol-5-yl]piperidin-4- yl}methyl)piperazin-1-yl]pyrimidin-4- yl}amino)-1-methyl-2-oxo-1,2- dihydroquinolin-3-yl]oxy}-N- methylacetamide |
| 68 | 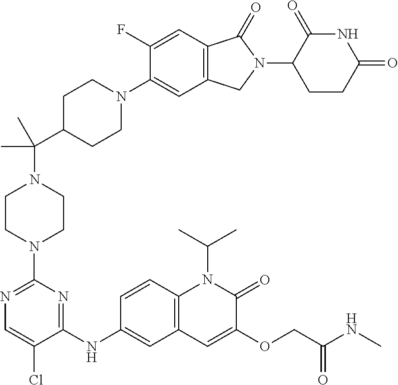 [SMILES: CNC(=O)COc1cc2cc(Nc3nc(N4CCN(C(C)(C)C5CCN(c6cc7c(cc6F)C(=O)N(C6CCC(=O)NC6=O)C7)CC5)CC4)ncc3Cl)ccc2n(C(C)C)c1=O] | 2-{[6-({5-chloro-2-[4-(2-{1-[2-(2,6- dioxopiperidin-3-yl)-6-fluoro-1-oxo-2,3- dihydro-1H-isoindol-5-yl]piperidin-4- yl}propan-2-yl)piperazin-1-yl]pyrimidin- 4-yl}amino)-2-oxo-1-(propan-2-yl)-1,2- dihydroquinolin-3-yl]oxy}-N- methylacetamide |
| 69 | 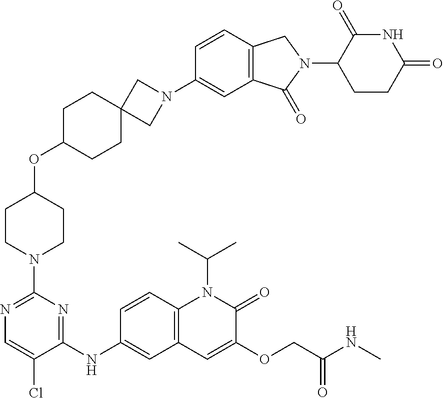 [SMILES: CNC(=O)COc1cc2cc(Nc3nc(N4CCC(OC5CCC6(CC5)CN(c5ccc7c(c5)C(=O)N(C5CCC(=O)NC5=O)C7)C6)CC4)ncc3Cl)ccc2n(C(C)C)c1=O] | 2-{[6-({5-chloro-2-[4-({2-[2-(2,6- dioxopiperidin-3-yl)-3-oxo-2,3-dihydro- 1H-isoindol-5-yl]-2-azaspiro[3.5]nonan-7- yl}oxy)piperidin-1-yl]pyrimidin-4- yl}amino)-2-oxo-1-(propan-2-yl)-1,2- dihydroquinolin-3-yl]oxy}-N- methylacetamide |
| 70 | 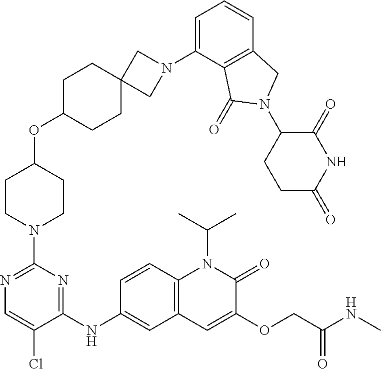 [SMILES: CNC(=O)COc1cc2cc(Nc3nc(N4CCC(OC5CCC6(CC5)CN(c5cccc7c5C(=O)N(C5CCC(=O)NC5=O)C7)C6)CC4)ncc3Cl)ccc2n(C(C)C)c1=O] | 2-{[6-({5-chloro-2-[4-({2-[2-(2,6- dioxopiperidin-3-yl)-3-oxo-2,3-dihydro- 1H-isoindol-4-yl]-2-azaspiro[3.5]nonan-7- yl}oxy)piperidin-1-yl]pyrimidin-4- yl}amino)-2-oxo-1-(propan-2-yl)-1,2- dihydroquinolin-3-yl]oxy}-N- methylacetamide |
| 71 | 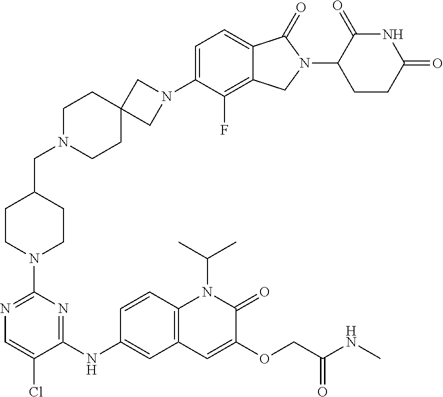 [SMILES: CNC(=O)COc1cc2cc(Nc3nc(N4CCC(CN5CCC6(CC5)CN(c5ccc7c(c5F)CN(C5CCC(=O)NC5=O)C7=O)C6)CC4)ncc3Cl)ccc2n(C(C)C)c1=O] | 2-{[6-({5-chloro-2-[4-({2-[2-(2,6- dioxopiperidin-3-yl)-4-fluoro-1-oxo-2,3- dihydro-1H-isoindol-5-yl]-27- diazaspiro[3.5]nonan-7- yl}methyl)piperidin-1-yl]pyrimidin-4- yl}amino)-2-oxo-1-(propan-2-yl)-1,2- dihydroquinolin-3-yl]oxy}-N- methylacetamide |
| 72 | 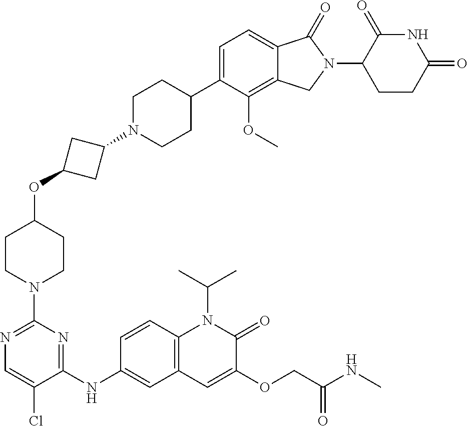 [SMILES: CNC(=O)COc1cc2cc(Nc3nc(N4CCC(O[C@H]5C[C@H](N6CCC(c7ccc8c(c7OC)CN(C7CCC(=O)NC7=O)C8=O)CC6)C5)CC4)ncc3Cl)ccc2n(C(C)C)c1=O] | 2-({6-[(5-chloro-2-{4-[(1r,3r)-3-{4-[2- (2,6-dioxopiperidin-3-yl)-4-methoxy-1- oxo-2,3-dihydro-1H-isoindol-5- yl]piperidin-1-yl}cyclobutoxy]piperidin- 1-yl}pyrimidin-4-yl)amino]-2-oxo-1- (propan-2-yl)-1,2-dihydroquinolin-3- yl}oxy)-N-methylacetamide |
| 73 | 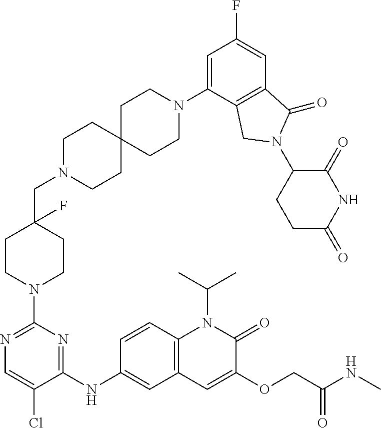 [SMILES: CNC(=O)COc1cc2cc(Nc3nc(N4CCC(F)(CN5CCC6(CC5)CCN(c5cc(F)cc7c5CN(C5CCC(=O)NC5=O)C7=O)CC6)CC4)ncc3Cl)ccc2n(C(C)C)c1=O] | 2-{[6-({5-chloro-2-[4-({9-[2-(2,6- dioxopiperidin-3-yl)-6-fluoro-1-oxo-2,3- dihydro-1H-isoindol-4-yl]-3,9- diazaspiro[5.5]undecan-3-yl}methyl)-4- fluoropiperidin-1-yl]pyrimidin-4- yl}amino)-2-oxo-1-(propan-2-yl)-1,2- dihydroquinolin-3-yl]oxy}-N- methylacetamide |
| 74 | 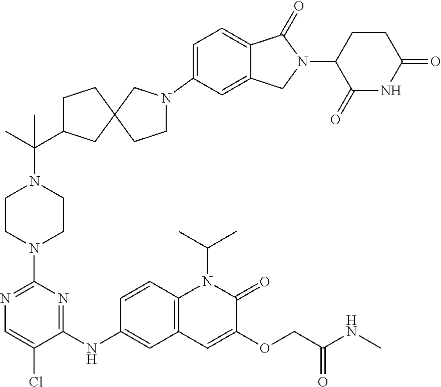 [SMILES: CNC(=O)COc1cc2cc(Nc3nc(N4CCN(C(C)(C)C5CCC6(CCN(c7ccc8c(c7)CN(C7CCC(=O)NC7=O)C8=O)C6)C5)CC4)ncc3Cl)ccc2n(C(C)C)c1=O] | 2-{[6-({5-chloro-2-[4-(2-{2-[2-(2,6- dioxopiperidin-3-yl)-1-oxo-2,3-dihydro- 1H-isoindol-5-yl]-2-azaspiro[4.4]nonan-7- yl}propan-2-yl)piperazin-1-yl]pyrimidin- 4-yl}amino)-2-oxo-1-(propan-2-yl)-1,2- dihydroquinolin-3-yl]oxy}-N- methylacetamide |
| 75 | 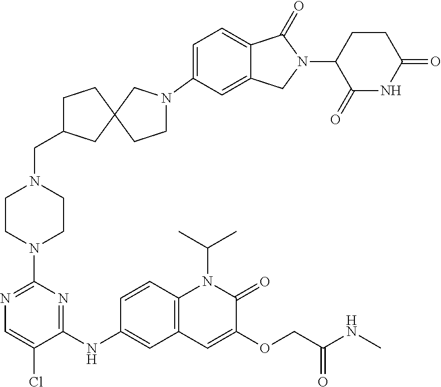 [SMILES: CNC(=O)COc1cc2cc(Nc3nc(N4CCN(CC5CCC6(CCN(c7ccc8c(c7)CN(C7CCC(=O)NC7=O)C8=O)C6)C5)CC4)ncc3Cl)ccc2n(C(C)C)c1=O] | 2-{[6-({5-chloro-2-[4-({2-[2-(2,6- dioxopiperidin-3-yl)-1-oxo-2,3-dihydro- 1H-isoindol-5-yl]-2-azaspiro[4.4]nonan-7- yl}methyl)piperazin-1-yl]pyrimidin-4- yl}amino)-2-oxo-1-(propan-2-yl)-1,2- dihydroquinolin-3-yl]oxy}-N- methylacetamide |
| 76 | 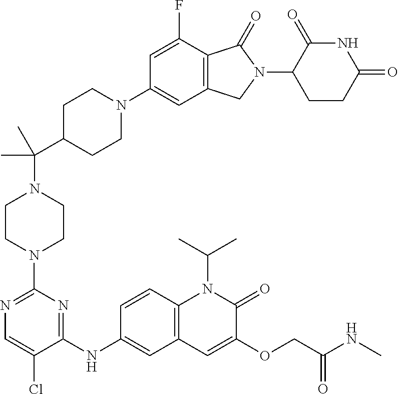 [SMILES: CNC(=O)COc1cc2cc(Nc3nc(N4CCN(C(C)(C)C5CCN(c6cc(F)c7c(c6)CN(C6CCC(=O)NC6=O)C7=O)CC5)CC4)ncc3Cl)ccc2n(C(C)C)c1=O] | 2-{[6-({5-chloro-2-[4-(2-{1-[2-(2,6- dioxopiperidin-3-yl)-7-fluoro-1-oxo-2,3- dihydro-1H-isoindol-5-yl]piperidin-4- yl}propan-2-yl)piperazin-1-yl]pyrimidin- 4-yl}amino)-2-oxo-1-(propan-2-yl)-1,2- dihydroquinolin-3-yl]oxy}-N- methylacetamide |
| 77 | 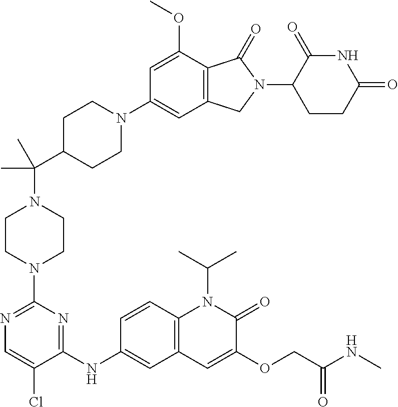 [SMILES: CNC(=O)COc1cc2cc(Nc3nc(N4CCN(C(C)(C)C5CCN(c6cc7c(c(OC)c6)C(=O)N(C6CCC(=O)NC6=O)C7)CC5)CC4)ncc3Cl)ccc2n(C(C)C)c1=O] | 2-{[6-({5-chloro-2-[4-(2-{1-[2-(2,6- dioxopiperidin-3-yl)-7-methoxy-1-oxo- 2,3-dihydro-1H-isoindol-5-yl]piperidin-4- yl}propan-2-yl)piperazin-1-yl]pyrimidin- 4-yl}amino)-2-oxo-1-(propan-2-yl)-1,2- dihydroquinolin-3-yl]oxy}-N- methylacetamide |
| 78 | 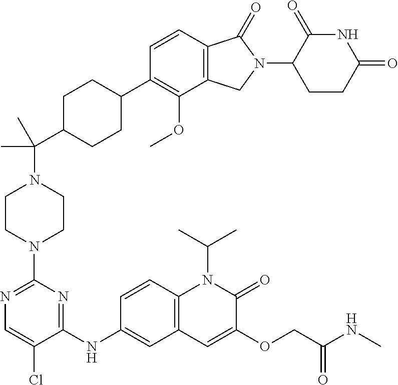 [SMILES: CNC(=O)COc1cc2cc(Nc3nc(N4CCN(C(C)(C)C5CCC(c6ccc7c(c6OC)CN(C6CCC(=O)NC6=O)C7=O)CC5)CC4)ncc3Cl)ccc2n(C(C)C)c1=O] | 2-{[6-({5-chloro-2-[4-(2-{1-[2-(2,6- dioxopiperidin-3-yl)-4-methoxy-1-oxo- 2,3-dihydro-1H-isoindol-5-yl]piperidin-4- yl}propan-2-yl)piperazin-1-yl]pyrimidin- 4-yl}amino)-2-oxo-1-(propan-2-yl)-1,2- dihydroquinolin-3-yl]oxy}-N- methylacetamide |
| 79 | 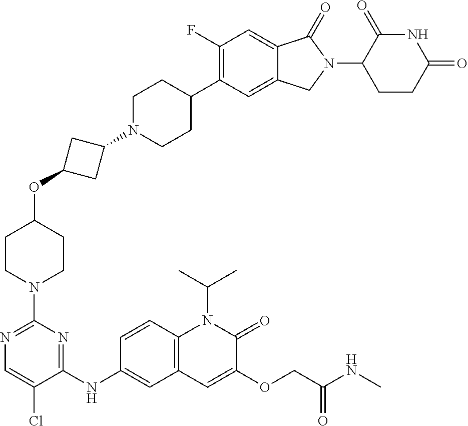 [SMILES: CNC(=O)COc1cc2cc(Nc3nc(N4CCC(O[C@H]5C[C@H](N6CCC(c7cc8c(cc7F)C(=O)N(C7CCC(=O)NC7=O)C8)CC6)C5)CC4)ncc3Cl)ccc2n(C(C)C)c1=O] | 2-({6-[(5-chloro-2-{4-[(1r,3r)-3-{4-[2- (2,6-dioxopiperidin-3-yl)-6-fluoro-1-oxo- 2,3-dihydro-1H-isoindol-5-yl]piperidin-1- yl}cyclobutoxy]piperidin-1-yl}pyrimidin- 4-yl)amino]-2-oxo-1-(propan-2-yl)-1,2- dihydroquinolin-3-yl}oxy)-N- methylacetamide |
| 80 | 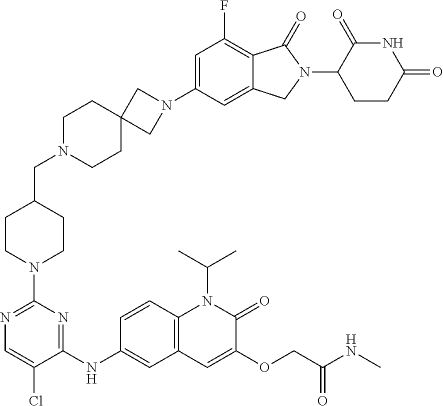 [SMILES: CNC(=O)COc1cc2cc(Nc3nc(N4CCC(CN5CCC6(CC5)CN(c5cc(F)c7c(c5)CN(C5CCC(=O)NC5=O)C7=O)C6)CC4)ncc3Cl)ccc2n(C(C)C)c1=O] | 2-{[6-({5-chloro-2-[4-({2-[2-(2,6- dioxopiperidin-3-yl)-7-fluoro-1-oxo-2,3- dihydro-1H-isoindol-5-yl]-2,7- diazaspiro[3.5]nonan-7- yl}methyl)piperidin-1-yl]pyrimidin-4- yl}amino)-2-oxo-1-(propan-2-yl)-1,2- dihydroquinolin-3-yl]oxy}-N- methylacetamide |
| 81 (Com- pound A) | 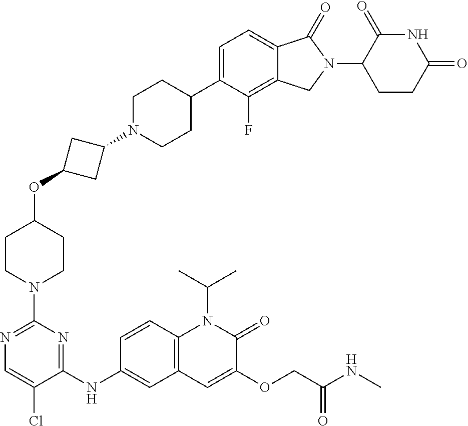 [SMILES: CNC(=O)COc1cc2cc(Nc3nc(N4CCC(O[C@H]5C[C@H](N6CCC(c7ccc8c(c7F)CN(C7CCC(=O)NC7=O)C8=O)CC6)C5)CC4)ncc3Cl)ccc2n(C(C)C)c1=O] | 2-({6-[(5-chloro-2-{4-[(1r,3r)-3-{4-[2- (2,6-dioxopiperidin-3-yl)-4-fluoro-1-oxo- 2,3-dihydro-1H-isoindol-5-yl]piperidin-1- yl}cyclobutoxy]piperidin-1-yl}pyrimidin- 4-yl)amino]-2-oxo-1-(propan-2-yl)-1,2- dihydroquinolin-3-yl}oxy)-N- methylacetamide |
| 82 | 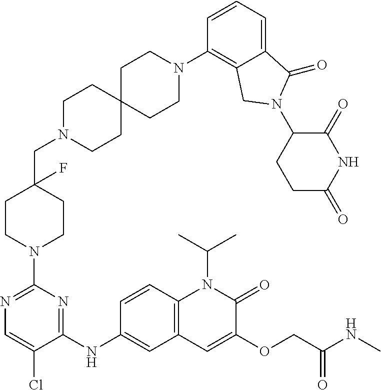 [SMILES: CNC(=O)COc1cc2cc(Nc3nc(N4CCC(F)(CN5CCC6(CC5)CCN(c5cccc7c5CN(C5CCC(=O)NC5=O)C7=O)CC6)CC4)ncc3Cl)ccc2n(C(C)C)c1=O] | 2-{[6-({5-chloro-2-[4-({9-[2-(2,6- dioxopiperidin-3-yl)-1-oxo-2,3-dihydro- 1H-isoindol-4-yl]-3,9- diazaspiro[5.5]undecan-3-yl}methyl)-4- fluoropiperidin-1-yl]pyrimidin-4- yl}amino)-2-oxo-1-(propan-2-yl)-1,2- dihydroquinolin-3-yl]oxy}-N- methylacetamide |
| 83 | 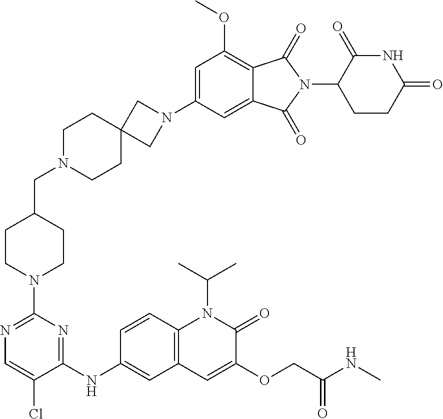 [SMILES: CNC(=O)COc1cc2cc(Nc3nc(N4CCC(CN5CCC6(CC5)CN(c5cc(OC)c7c(c5)C(=O)N(C5CCC(=O)NC5=O)C7=O)C6)CC4)ncc3Cl)ccc2n(C(C)C)c1=O] | 2-{[6-({5-chloro-2-[4-({2-[2-(2,6- dioxopiperidin-3-yl)-7-methoxy-1,3- dioxo-2,3-dihydro-1H-isoindol-5-yl]-2,7- diazaspiro[3.5]nonan-7- yl}methyl)piperidin-1-yl]pyrimidin-4- yl}amino)-2-oxo-1-(propan-2-yl)-1,2- dihydroquinolin-3-yl]oxy}-N- methylacetamide |
| 84 | 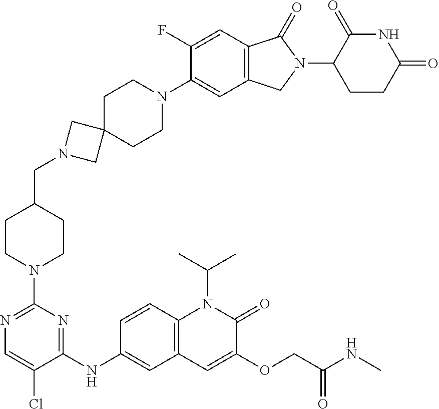 [SMILES: CNC(=O)COc1cc2cc(Nc3nc(N4CCC(CN5CC6(CCN(c7cc8c(cc7F)C(=O)N(C7CCC(=O)NC7=O)C8)CC6)C5)CC4)ncc3Cl)ccc2n(C(C)C)c1=O] | 2-{[6-({5-chloro-2-[4-({7-[2-(2,6- dioxopiperidin-3-yl)-6-fluoro-1-oxo-2,3- dihydro-1H-isoindol-5-yl]-2,7- diazaspiro[3.5]nonan-2- yl}methyl)piperidin-1-yl]pyrimidin-4- yl}amino)-2-oxo-1-(propan-2-yl)-1,2- dihydroquinolin-3-yl]oxy}-N- methylacetamide |
| 85 | 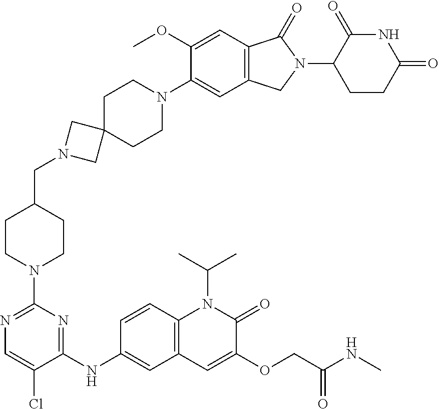 [SMILES: CNC(=O)COc1cc2cc(Nc3nc(N4CCC(CN5CC6(CCN(c7cc8c(cc7OC)C(=O)N(C7CCC(=O)NC7=O)C8)CC6)C5)CC4)ncc3Cl)ccc2n(C(C)C)c1=O] | 2-{[6-({5-chloro-2-[4-({7-[2-(2,6- dioxopiperidin-3-yl)-6-methoxy-1-oxo- 2,3-dihydro-1H-isoindol-5-yl]-2,7- diazaspiro[3.5]nonan-2- yl}methyl)piperidin-1-yl]pyrimidin-4- yl}amino)-2-oxo-1-(propan-2-yl)-1,2- dihydroquinolin-3-yl]oxy}-N- methylacetamide |
| 86 | 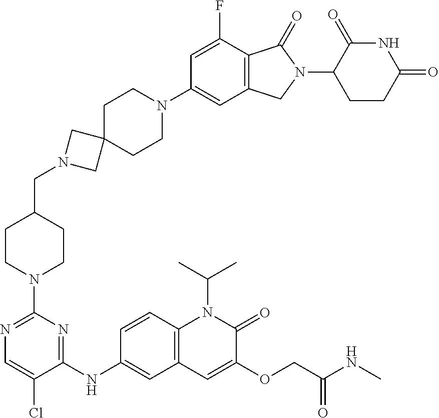 [SMILES: CNC(=O)COc1cc2cc(Nc3nc(N4CCC(CN5CC6(CCN(c7cc(F)c8c(c7)CN(C7CCC(=O)NC7=O)C8=O)CC6)C5)CC4)ncc3Cl)ccc2n(C(C)C)c1=O] | 2-{[6-({5-chloro-2-[4-({7-[2-(2,6- dioxopiperidin-3-yl)-7-fluoro-1-oxo-2,3- dihydro-1H-isoindol-5-yl]-2,7- diazaspiro[3.5]nonan-2- yl}methyl)piperidin-1-yl]pyrimidin-4- yl}amino)-2-oxo-1-(propan-2-yl)-1,2- dihydroquinolin-3-yl]oxy}-N- methylacetamide |
| 87 | 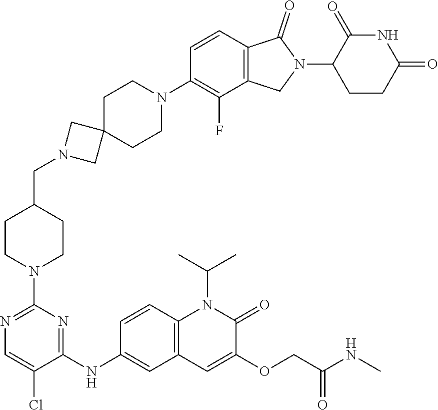 [SMILES: CNC(=O)COc1cc2cc(Nc3nc(N4CCC(CN5CC6(CCN(c7ccc8c(c7F)CN(C7CCC(=O)NC7=O)C8=O)CC6)C5)CC4)ncc3Cl)ccc2n(C(C)C)c1=O] | 2-{[6-({5-chloro-2-[4-({7-[2-(2,6- dioxopiperidin-3-yl)-4-fluoro-1-oxo-2,3- dihydro-1H-isoindol-5-yl]-2,7- diazaspiro[3.5]nonan-2- yl}methyl)piperidin-1-yl]pyrimidin-4- yl}amino)-2-oxo-1-(propan-2-yl)-1,2- dihydroquinolin-3-yl]oxy}-N- methylacetamide |
| 88 | 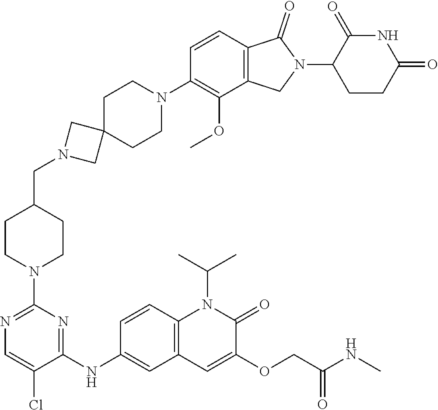 [SMILES: CNC(=O)COc1cc2cc(Nc3nc(N4CCC(CN5CC6(CCN(c7ccc8c(c7OC)CN(C7CCC(=O)NC7=O)C8=O)CC6)C5)CC4)ncc3Cl)ccc2n(C(C)C)c1=O] | 2-{[6-({5-chloro-2-[4-({7-[2-(2,6- dioxopiperidin-3-yl)-4-methoxy-1-oxo- 2,3-dihydro-1H-isoindol-5-yl]-2,7- diazaspiro[3.5]nonan-2- yl}methyl)piperidin-1-yl]pyrimidin-4- yl}amino)-2-oxo-1-(propan-2-yl)-1,2- dihydroquinolin-3-yl]oxy}-N- methylacetamide |
| 89 | 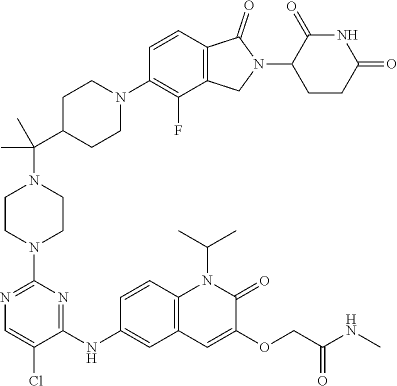 [SMILES: CNC(=O)COc1cc2cc(Nc3nc(N4CCN(C(C)(C)C5CCN(c6ccc7c(c6F)CN(C6CCC(=O)NC6=O)C7=O)CC5)CC4)ncc3Cl)ccc2n(C(C)C)c1=O] | 2-{[6-({5-chloro-2-[4-(2-{1-[2-(2,6- dioxopiperidin-3-yl)-4-fluoro-1-oxo-2,3- dihydro-1H-isoindol-5-yl]piperidin-4- yl}propan-2-yl)piperazin-1-yl]pyrimidin- 4-yl}amino)-2-oxo-1-(propan-2-yl)-1,2- dihydroquinolin-3-yl]oxy}-N- methylacetamide |
| 90 | 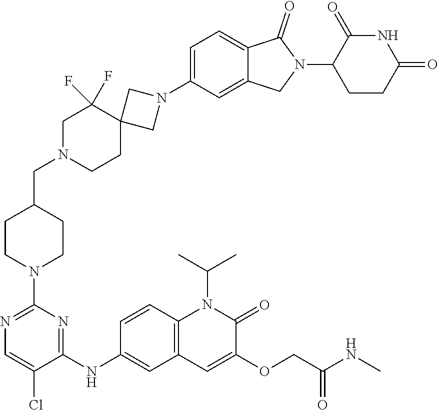 [SMILES: CNC(=O)COc1cc2cc(Nc3nc(N4CCC(CN5CCC6(CN(c7ccc8c(c7)CN(C7CCC(=O)NC7=O)C8=O)C6)C(F)(F)C5)CC4)ncc3Cl)ccc2n(C(C)C)c1=O] | 2-{[6-({5-chloro-2-[4-({2-[2-(2,6- dioxopiperidin-3-yl)-1-oxo-2,3-dihydro- 1H-isoindol-5-yl]-5,5-difluoro-2,7- diazaspiro[3.5]nonan-7- yl}methyl)piperidin-1-yl]pyrimidin-4- yl}amino)-2-oxo-1-(propan-2-yl)-1,2- dihydroquinolin-3-yl]oxy}-N- methylacetamide |
| 91 | 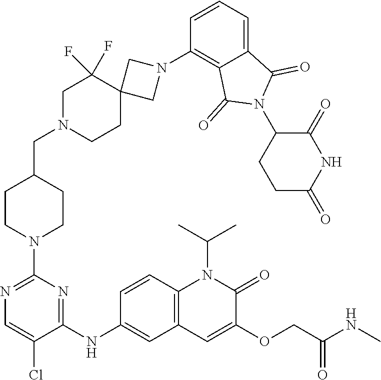 [SMILES: CNC(=O)COc1cc2cc(Nc3nc(N4CCC(CN5CCC6(CN(c7cccc8c7C(=O)N(C7CCC(=O)NC7=O)C8=O)C6)C(F)(F)C5)CC4)ncc3Cl)ccc2n(C(C)C)c1=O] | 2-{[6-({5-chloro-2-[4-({2-[2-(2,6- dioxopiperidin-3-yl)-1,3-dioxo-2,3- dihydro-1H-isoindol-4-yl]-5,5-difluroo- 2,7-diazaspiro[3.5]nonan-7- yl}methyl)piperidin-1-yl]pyrimidin-4- yl}amino)-2-oxo-1-(propan-2-yl)-1,2- dihydroquinolin-3-yl]oxy}-N- methylacetamide |
| 92 | 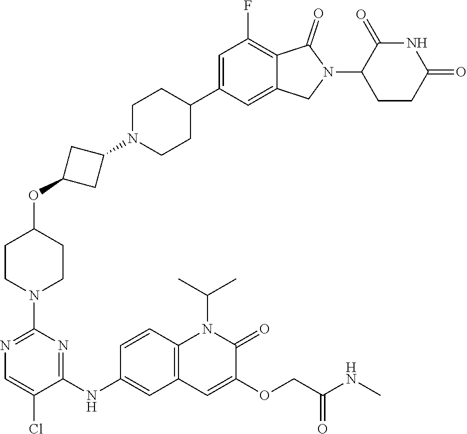 [SMILES: CNC(=O)COc1cc2cc(Nc3nc(N4CCC(O[C@H]5C[C@H](N6CCC(c7cc(F)c8c(c7)CN(C7CCC(=O)NC7=O)C8=O)CC6)C5)CC4)ncc3Cl)ccc2n(C(C)C)c1=O] | 2-({6-[(5-chloro-2-{4-[(1r,3r)-3-{4-[2- (2,6-dioxopiperidin-3-yl)-7-fluoro-1-oxo- 2,3-dihydro-1H-isoindol-5-yl]piperidin-1- yl}cyclobutoxy]piperidin-1-yl}pyrimidin- 4-yl)amino]-2-oxo-1-(propan-2-yl)-1,2- dihydroquinolin-3-yl}oxy)-N- methylacetamide |
| 93 | 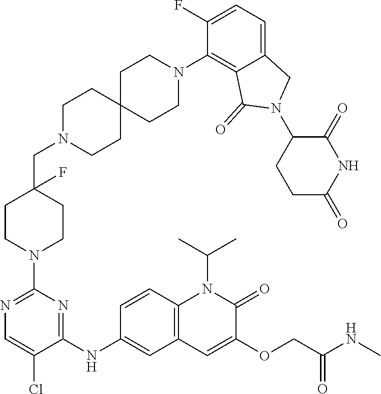 [SMILES: CNC(=O)COc1cc2cc(Nc3nc(N4CCC(F)(CN5CCC6(CC5)CCN(c5c(F)ccc7c5C(=O)N(C5CCC(=O)NC5=O)C7)CC6)CC4)ncc3Cl)ccc2n(C(C)C)c1=O] | 2-{[6-({5-chloro-2-[4-({9-[2-(2,6- dioxopiperidin-3-yl)-5-fluoro-3-oxo-2,3- dihydro-1H-isoindol-4-yl]-3,9- diazaspiro[5.5]undecan-3-yl}methyl)-4- fluoropiperidin-1-yl]pyrimidin-4- yl}amino)-2-oxo-1-(propan-2-yl)-1,2- dihydroquinolin-3-yl]oxy}-N- methylacetamide |
| 94 | 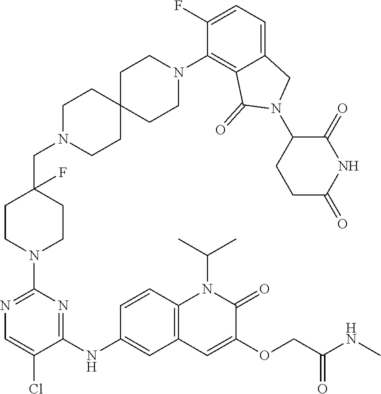 [SMILES: CNC(=O)COc1cc2cc(Nc3nc(N4CCC(F)(CN5CCC6(CC5)CCN(c5c(F)ccc7c5C(=O)N(C5CCC(=O)NC5=O)C7)CC6)CC4)ncc3Cl)ccc2n(C(C)C)c1=O] | 2-{[6-({5-chloro-2-[4-({9-[2-(2,6- dioxopiperidin-3-yl)-7-fluoro-1-oxo-2,3- dihydro-1H-isoindol-4-yl]-3,9- diazaspiro[5.5]undecan-3-yl}methyl)-4- fluoropiperidin-1-yl]pyrimidin-4- yl}amino)-2-oxo-1-(propan-2-yl)-1,2- dihydroquinolin-3-yl]oxy}-N- methylacetamide |
| 95 | 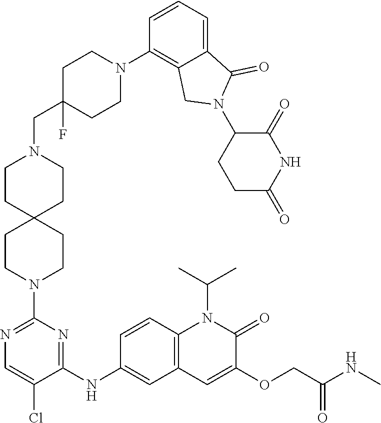 [SMILES: CNC(=O)COc1cc2cc(Nc3nc(N4CCC5(CCN(CC6(F)CCN(c7cccc8c7CN(C7CCC(=O)NC7=O)C8=O)CC6)CC5)CC4)ncc3Cl)ccc2n(C(C)C)c1=O] | 2-{[6-({5-chloro-2-[9-({1-[2-(2,6- dioxopiperidin-3-yl)-1-oxo-2,3-dihydro- 1H-isoindol-4-yl]-4-fluoropiperidin-4- yl}methyl)-3,9-diazaspiro[5.5]undecan-3- yl]pyrimidin-4-yl}amino)-2-oxo-1- (propan-2-yl)-1,2-dihydroquinolin-3- yl]oxy}-N-methylacetamide |
| 96 | 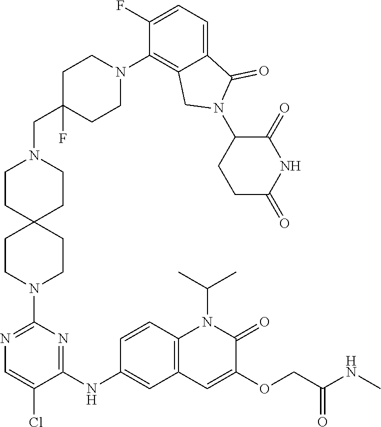 [SMILES: CNC(=O)COc1cc2cc(Nc3nc(N4CCC5(CCN(CC6(F)CCN(c7c(F)ccc8c7CN(C7CCC(=O)NC7=O)C8=O)CC6)CC5)CC4)ncc3Cl)ccc2n(C(C)C)c1=O] | 2-{[6-({5-chloro-2-[9-({1-[2-(2,6- dioxopiperidin-3-yl)-5-fluoro-1-oxo-2,3- dihydro-1H-isoindol-4-yl]-4- fluoropiperidin-4-yl}methyl)-3,9- diazaspiro[5.5]undecan-3-yl]pyrimidin-4- yl}amino)-2-oxo-1-(propan-2-yl)-1,2- dihydroquinolin-3-yl]oxy}-N- methylacetamide |
| 97 | 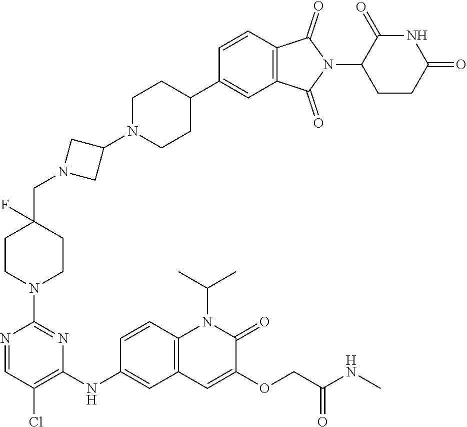 [SMILES: CNC(=O)COc1cc2cc(Nc3nc(N4CCC(F)(CN5CC(N6CCC(c7ccc8c(c7)C(=O)N(C7CCC(=O)NC7=O)C8=O)CC6)C5)CC4)ncc3Cl)ccc2n(C(C)C)c1=O] | 2-({6-[(5-chloro-2-{4-[(3-{4-[2-(2,6- dioxopiperidin-3-yl)-1,3-dioxo-2,3- dihydro-1H-isoindol-5-yl]piperidin-1- yl}azetidin-1-yl)methyl]-4- fluoropiperidin-1-yl}pyrimidin-4- yl)amino]-2-oxo-1-(propan-2-yl)-1,2- dihydroquinolin-3-yl}oxy)-N- methylacetamide |
| 98 | 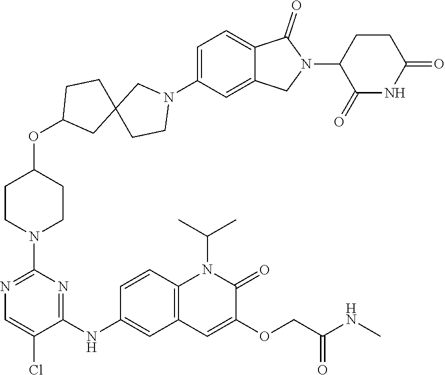 [SMILES: CNC(=O)COc1cc2cc(Nc3nc(N4CCC(OC5CCC6(CCN(c7ccc8c(c7)CN(C7CCC(=O)NC7=O)C8=O)C6)C5)CC4)ncc3Cl)ccc2n(C(C)C)c1=O] | 2-{[6-({5-chloro-2-[4-({2-[2-(2,6- dioxopiperidin-3-yl)-1-oxo-2,3-dihydro- 1H-isoindol-5-yl]-2-azaspiro[4.4]nonan-7- yl}oxy)piperidin-1-yl]pyrimidin-4- yl}amino)-2-oxo-1-(propan-2-yl)-1,2- dihydroquinolin-3-yl]oxy}-N- methylacetamide |
| 99 | 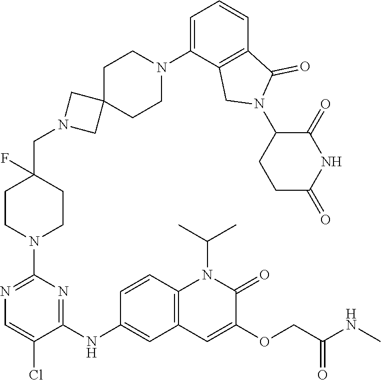 [SMILES: CNC(=O)COc1cc2cc(Nc3nc(N4CCC(F)(CN5CC6(CCN(c7cccc8c7CN(C7CCC(=O)NC7=O)C8=O)CC6)C5)CC4)ncc3Cl)ccc2n(C(C)C)c1=O] | 2-{[6-({5-chloro-2-[4-({7-[2-(2,6- dioxopiperidin-3-yl)-1-oxo-2,3-dihydro- 1H-isoindol-4-yl]-2,7- diazaspiro[3.5]nonan-2-yl}methyl)-4- fluoropiperidin-1-yl]pyrimidin-4- yl}amino)-2-oxo-1-(propan-2-yl)-1,2- dihydroquinolin-3-yl]oxy}-N- methylacetamide |
| 100 | 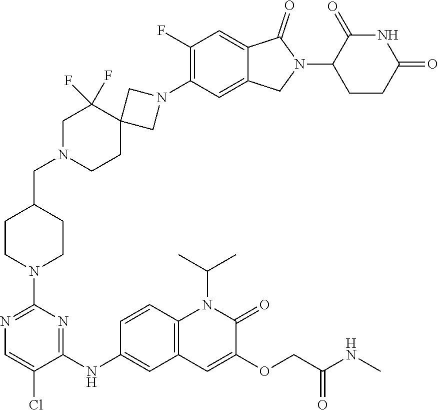 [SMILES: CNC(=O)COc1cc2cc(Nc3nc(N4CCC(CN5CCC6(CN(c7cc8c(cc7F)C(=O)N(C7CCC(=O)NC7=O)C8)C6)C(F)(F)C5)CC4)ncc3Cl)ccc2n(C(C)C)c1=O] | 2-{[6-({5-chloro-2-[4-({2-[2-(2,6- dioxopiperidin-3-yl)-6-fluoro-1-oxo-2,3- dihydro-1H-isoindol-5-yl]-5,5-difluoro- 2,7-diazaspiro[3.5]nonan-7- yl}methyl)piperidin-1-yl]pyrimidin-4- yl}amino)-2-oxo-1-(propan-2-yl)-1,2- dihydroquinolin-3-yl]oxy}-N- methylacetamide |
| 101 | 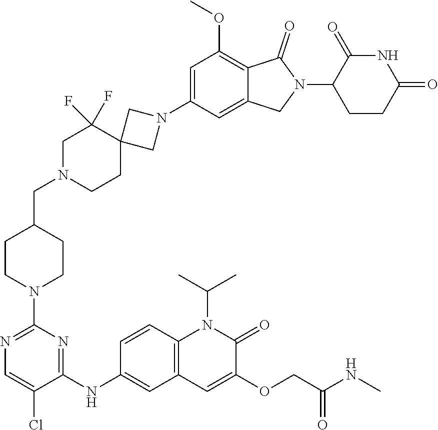 [SMILES: CNC(=O)COc1cc2cc(Nc3nc(N4CCC(CN5CCC6(CN(c7cc8c(c(OC)c7)C(=O)N(C7CCC(=O)NC7=O)C8)C6)C(F)(F)C5)CC4)ncc3Cl)ccc2n(C(C)C)c1=O] | 2-{[6-({5-chloro-2-[4-({2-[2-(2,6- dioxopiperidin-3-yl)-7-methoxy-1-oxo- 2,3-dihydro-1H-isoindol-5-yl]-5,5- difluoro-2,7-diazaspiro[3.5]nonan-7- yl}methyl)piperidin-1-yl]pyrimidin-4- yl}amino)-2-oxo-1-(propan-2-yl)-1,2- dihydroquinolin-3-yl]oxy}-N- methylacetamide |
| 102 | 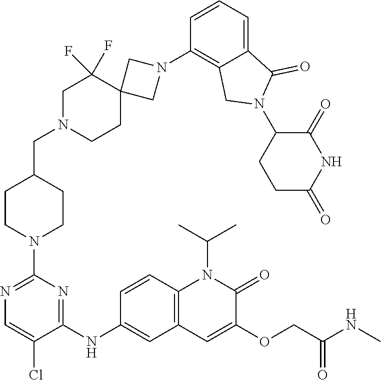 [SMILES: CNC(=O)COc1cc2cc(Nc3nc(N4CCC(CN5CCC6(CN(c7cccc8c7CN(C7CCC(=O)NC7=O)C8=O)C6)C(F)(F)C5)CC4)ncc3Cl)ccc2n(C(C)C)c1=O] | 2-{[6-({5-chloro-2-[4-({2-[2-(2,6- dioxopiperidin-3-yl)-1-oxo-2,3-dihydro- 1H-isoindol-4-yl]-5,5-difluoro-2,7- diazaspiro[3.5]nonan-7- yl}methyl)piperidin-1-yl]pyrimidin-4- yl}amino)-2-oxo-1-(propan-2-yl)-1,2- dihydroquinolin-3-yl]oxy}-N- methylacetamide |
| 103 | 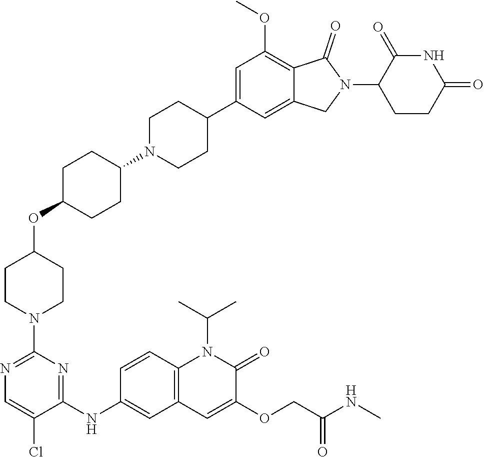 [SMILES: CNC(=O)COc1cc2cc(Nc3nc(N4CCC(O[C@H]5CC[C@H](N6CCC(c7cc8c(c(OC)c7)C(=O)N(C7CCC(=O)NC7=O)C8)CC6)CC5)CC4)ncc3Cl)ccc2n(C(C)C)c1=O] | 2-({6-[(5-chloro-2-{4-[(1r,3r)-3-{4-[2- (2,6-dioxopiperidin-3-yl)-7-methoxy-1- oxo-2,3-dihydro-1H-isoindol-5- yl]piperidin-1-yl}cyclobutoxy]piperidin- 1-yl}pyrimidin-4-yl)amino]-2-oxo-1- (propan-2-yl)-1,2-dihydroquinolin-3- yl}oxy)-N-methylacetamide |
| 104 | 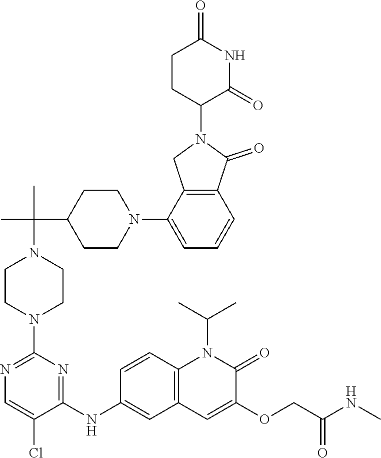 [SMILES: CNC(=O)COc1cc2cc(Nc3nc(N4CCN(C(C)(C)C5CCN(c6cccc7c6CN(C6CCC(=O)NC6=O)C7=O)CC5)CC4)ncc3Cl)ccc2n(C(C)C)c1=O] | 2-{[6-({5-chlroo-2-[4-(2-{1-[2-(2,6- dioxopiperidin-3-yl)-1-oxo-2,3-dihydro- 1H-isoindol-4-yl]piperidin-4-yl}propan-2- yl)piperazin-1-yl]pyrimidin-4-yl}amino)- 2-oxo-1-(propan-2-yl)-1,2- dihydroquinolin-3-yl]oxy}-N- methylacetamide |
| 105 | 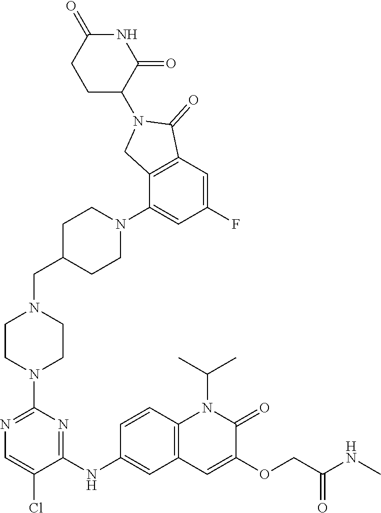 [SMILES: CNC(=O)COc1cc2cc(Nc3nc(N4CCN(CC5CCN(c6cc(F)cc7c6CN(C6CCC(=O)NC6=O)C7=O)CC5)CC4)ncc3Cl)ccc2n(C(C)C)c1=O] | 2-{[6-({5-chloro-2-[4-({1-[2-(2,6- dioxopiperidin-3-yl)-6-fluoro-1-oxo-2,3- dihydro-1H-isoindol-4-yl]piperidin-4- yl}methyl)piperazin-1-yl]pyrimidin-4- yl}amino)-2-oxo-1-(propan-2-yl)-1,2- dihydroquinolin-3-yl]oxy}-N- methylacetamide |
| 106 | 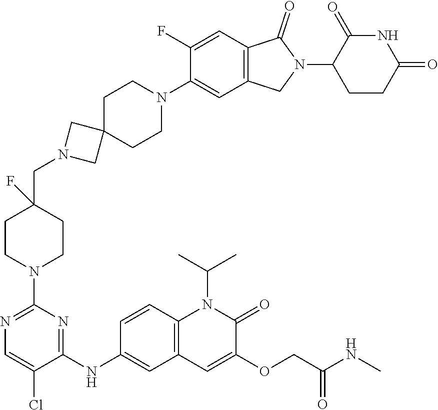 [SMILES: CNC(=O)COc1cc2cc(Nc3nc(N4CCC(F)(CN5CC6(CCN(c7cc8c(cc7F)C(=O)N(C7CCC(=O)NC7=O)C8)CC6)C5)CC4)ncc3Cl)ccc2n(C(C)C)c1=O] | 2-{[6-({5-chloro-2-[4-({7-[2-(2,6- dioxopiperidin-3-yl)-6-fluoro-1-oxo-2,3- dihydro-1H-isoindol-5-yl]-2,7- diazaspiro[3.5]nonan-2-yl}methyl)-4- fluoropiperidin-1-yl]pyrimidin-4- yl}amino)-2-oxo-1-(propan-2-yl)-1,2- dihydroquinolin-3-yl]oxy}-N- methylacetamide |
| 107 | 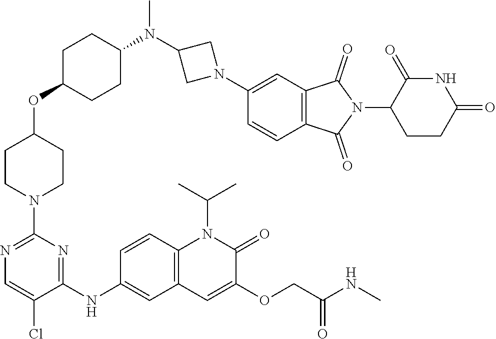 [SMILES: CNC(=O)COc1cc2cc(Nc3nc(N4CCC(O[C@H]5CC[C@H](N(C)C6CN(c7ccc8c(c7)C(=O)N(C7CCC(=O)NC7=O)C8=O)C6)CC5)CC4)ncc3Cl)ccc2n(C(C)C)c1=O] | 2-[(6-{[5-chloro-2-(4-{[(1r,4r)-4-({1-[2- (2,6-dioxopiperidin-3-yl)-1,3-dioxo-2,3- dihydro-1H-isoindol-5-yl]azetidin-3- yl}(methyl)amino)cyclohexyl]oxy}piperidin- 1-yl)pyrimidin-4-yl]amino}-2-oxo-1- (propan-2-yl)-1,2-dihydroquinolin-3- yl)oxy]-N-methylacetamide |
| 108 | 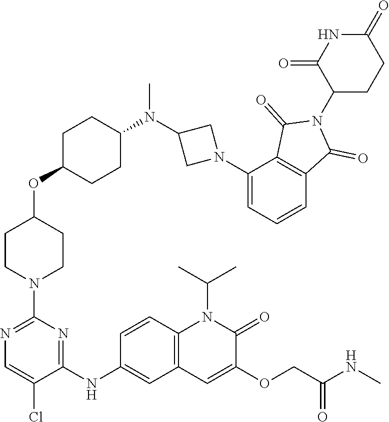 [SMILES: CNC(=O)COc1cc2cc(Nc3nc(N4CCC(O[C@H]5CC[C@H](N(C)C6CN(c7cccc8c7C(=O)N(C7CCC(=O)NC7=O)C8=O)C6)CC5)CC4)ncc3Cl)ccc2n(C(C)C)c1=O] | 2-[(6-{[5-chloro-2-(4-{[(1r,4r)-4-({1-[2- (2,6-dioxopiperidin-3-yl)-1,3-dioxo-2,3- dihydro-1H-isoindol-4-yl]azetidin-3- yl}(methyl)amino)cyclohexyl]oxy}piperidin- 1-yl)pyrimidin-4-yl]amino}-2-oxo-1- (propan-2-yl)-1,2-dihydroquinolin-3- yl)oxy]-N-methylacetamide |
| 109 | 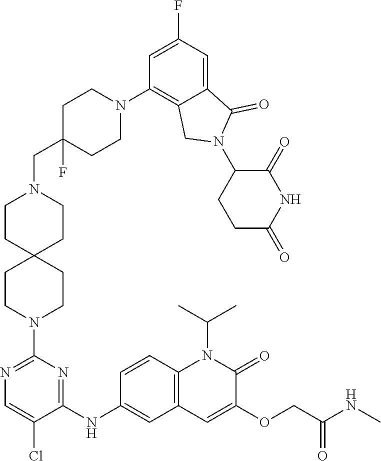 [SMILES: CNC(=O)COc1cc2cc(Nc3nc(N4CCC5(CCN(CC6(F)CCN(c7cc(F)cc8c7CN(C7CCC(=O)NC7=O)C8=O)CC6)CC5)CC4)ncc3Cl)ccc2n(C(C)C)c1=O] | 2-{[6-({5-chloro-2-[9-({1-[2-(2,6- dioxopiperidin-3-yl)-6-fluoro-1-oxo-2,3- dihydro-1H-isoindol-4-yl]-4- fluoropiperidin-4-yl}methyl)-3,9- diazaspiro[5.5]undecan-3-yl]pyrimidin-4- yl}amino)-2-oxo-1-(propan-2-yl)-1,2- dihydroquinolin-3-yl]oxy}-N- methylacetamide |
| 110 | 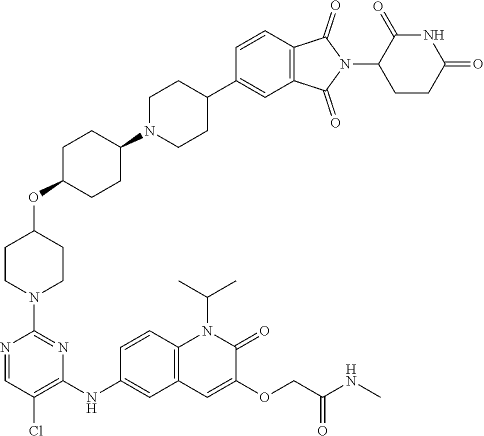 [SMILES: CNC(=O)COc1cc2cc(Nc3nc(N4CCC(O[C@H]5CC[C@@H](N6CCC(c7ccc8c(c7)C(=O)N(C7CCC(=O)NC7=O)C8=O)CC6)CC5)CC4)ncc3Cl)ccc2n(C(C)C)c1=O] | 2-[(6-{[5-chloro-2-(4-{[(1s,4s)-4-{4-[2- (2,6-dioxopiperidin-3-yl)-1,3-dioxo-2,3- dihydro-1H-isoindol-5-yl]piperidin-1- yl}cyclohexyl]oxy}piperidin-1- yl)pyrimidin-4-yl]amino}-2-oxo-1- (propan-2-yl)-1,2-dihydroquinolin-3- yl)oxy]-N-methylacetamide |
| 111 | 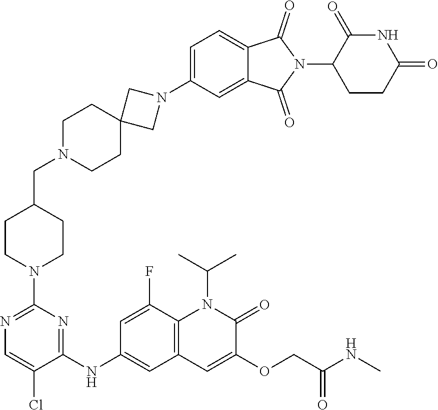 [SMILES: CNC(=O)COc1cc2cc(Nc3nc(N4CCC(CN5CCC6(CC5)CN(c5ccc7c(c5)C(=O)N(C5CCC(=O)NC5=O)C7=O)C6)CC4)ncc3Cl)cc(F)c2n(C(C)C)c1=O] | 2-{[6-({5-chloro-2-[4-({2-[2-(2,6- dioxopiperidin-3-yl)-1,3-dioxo-2,3- dihydro-1H-isoindol-5-yl]-2,7- diazaspiro[3.5]nonan-7- yl}methyl)piperidin-1-yl]pyrimidin-4- yl}amino)-8-fluoro-2-oxo-1-(propan-2- yl)-1,2-dihydroquinolin-3-yl]oxy}-N- methylacetamide |
| 112 | 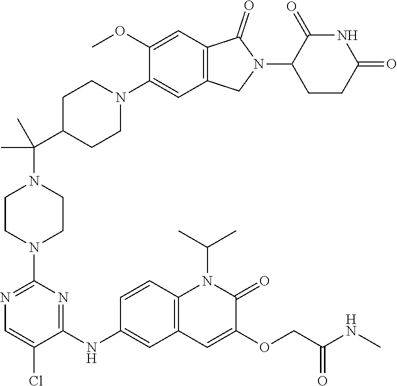 [SMILES: CNC(=O)COc1cc2cc(Nc3nc(N4CCN(C(C)(C)C5CCN(c6cc7c(cc6OC)C(=O)N(C6CCC(=O)NC6=O)C7)CC5)CC4)ncc3Cl)ccc2n(C(C)C)c1=O] | 2-{[6-({5-chloro-2-[4-(2-{1-[2-(2,6- dioxopiperidin-3-yl)-6-methoxy-1-oxo- 2,3-dihydro-1H-isoindol-5-yl]piperidin-4- yl}propan-2-yl)piperazin-1-yl]pyrimidin- 4-yl}amino)-2-oxo-1-(propan-2-yl)-1,2- dihydroquinolin-3-yl]oxy}-N- methylacetamide |
| 113 | 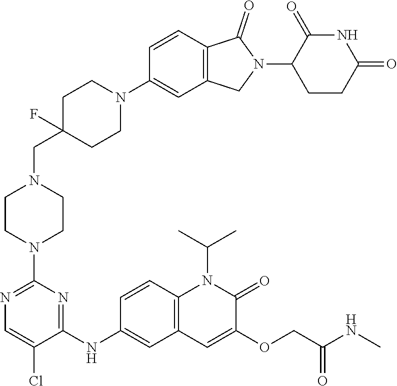 [SMILES: CNC(=O)COc1cc2cc(Nc3nc(N4CCN(CC5(F)CCN(c6ccc7c(c6)CN(C6CCC(=O)NC6=O)C7=O)CC5)CC4)ncc3Cl)ccc2n(C(C)C)c1=O] | 2-{[6-({5-chloro-2-[4-({1-[2-(2,6- dioxopiperidin-3-yl)-1-oxo-2,3-dihydro- 1H-isoindol-5-yl]-4-fluoropiperidin-4- yl}methyl)piperazin-1-yl]pyrimidin-4- yl}amino)-2-oxo-1-(propan-2-yl)-1,2- dihydroquinolin-3-yl]oxy}-N- methylacetamide |
| 114 | 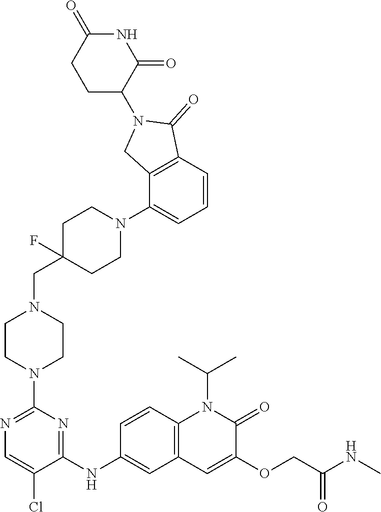 [SMILES: CNC(=O)COc1cc2cc(Nc3nc(N4CCN(CC5(F)CCN(c6cccc7c6CN(C6CCC(=O)NC6=O)C7=O)CC5)CC4)ncc3Cl)ccc2n(C(C)C)c1=O] | 2-{[6-({5-chloro-2-[4-({1-[2-(2,6- dioxopiperidin-3-yl)-1-oxo-2,3-dihydro- 1H-isoindol-4-yl]-4-fluoropiperidin-4- yl}methyl)piperazin-1-yl]pyrimidin-4- yl}amino)-2-oxo-1-(propan-2-yl)-1,2- dihydroquinolin-3-yl]oxy}-N- methylacetamide |
| 115 | 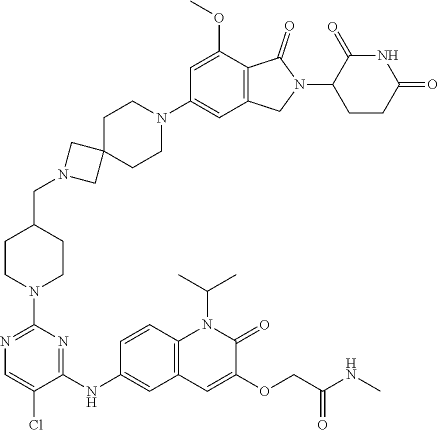 [SMILES: CNC(=O)COc1cc2cc(Nc3nc(N4CCC(CN5CC6(CCN(c7cc8c(c(OC)c7)C(=O)N(C7CCC(=O)NC7=O)C8)CC6)C5)CC4)ncc3Cl)ccc2n(C(C)C)c1=O] | 2-{[6-({5-chloro-2-[4-({7-[2-(2,6- dioxopiperidin-3-yl)-7-methoxy-1-oxo- 2,3-dihydro-1H-isoindol-5-yl]-2,7- diazaspiro[3.5]nonan-2- yl}methyl)piperidin-1-yl]pyrimidin-4- yl}amino)-2-oxo-1-(propan-2-yl)-1,2- dihydroquinolin-3-yl]oxy}-N- methylacetamide |
| 116 | 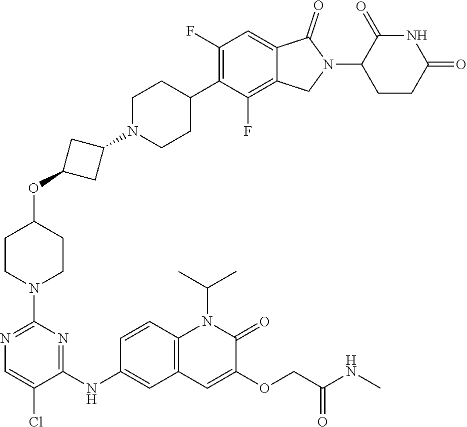 [SMILES: CNC(=O)COc1cc2cc(Nc3nc(N4CCC(O[C@H]5C[C@H](N6CCC(c7c(F)cc8c(c7F)CN(C7CCC(=O)NC7=O)C8=O)CC6)C5)CC4)ncc3Cl)ccc2n(C(C)C)c1=O] | 2-({6-[(5-chloro-2-{4-[(1r,3r)-3-{4-[2- (2,6-dioxopiperidin-3-yl)-4,6-difluoro-1- oxo-2,3-dihydro-1H-isoindol-5- yl]piperidin-1-yl}cyclobutoxy]piperidin- 1-yl}pyrimidin-4-yl)amino]-2-oxo-1- (propan-2-yl)-1,2-dihydroquinolin-3- yl}oxy)-N-methylacetamide |
| 117 | 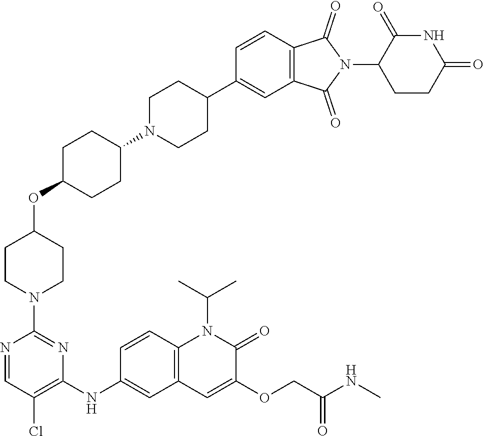 [SMILES: CNC(=O)COc1cc2cc(Nc3nc(N4CCC(O[C@H]5CC[C@H](N6CCC(c7ccc8c(c7)C(=O)N(C7CCC(=O)NC7=O)C8=O)CC6)CC5)CC4)ncc3Cl)ccc2n(C(C)C)c1=O] | 2-[(6-{[5-chloro-2-(4-{[(1r,4r)-4-{4-[2- (2,6-dioxopiperidin-3-yl)-1,3-dioxo-2,3- dihydro-1H-isoindol-5-yl]piperidin-1- yl}cyclohexyl]oxy}piperidin-1- yl)pyrimidin-4-yl]amino}-2-oxo-1- (propan-2-yl)-1,2-dihydroquinolin-3- yl)oxy]-N-methylacetamide |
| 118 | 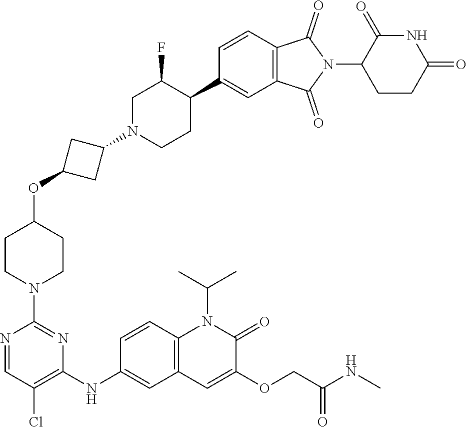 [SMILES: CNC(=O)COc1cc2cc(Nc3nc(N4CCC(O[C@H]5C[C@H](N6CC[C@H](c7ccc8c(c7)C(=O)N(C7CCC(=O)NC7=O)C8=O)[C@H](F)C6)C5)CC4)ncc3Cl)ccc2n(C(C)C)c1=O] | 2-({6-[(5-chloro-2-{4-[(1r,3r)-3-[(3S,4R)- 4-[2-(2,6-dioxopiperidin-3-yl)-1,3-dioxo- 2,3-dihydro-1H-isoindol-5-yl]-3- fluoropiperidin-1- yl]cyclobutoxy]piperidin-1-yl}pyrimidin- 4-yl)amino]-2-oxo-1-(propan-2-yl)-1,2- dihydroquinolin-3-yl}oxy)-N- methylacetamide |
| 119 | 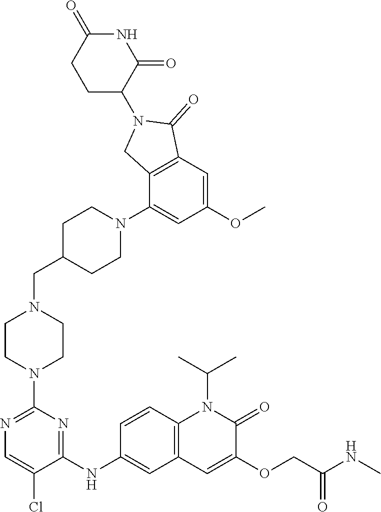 [SMILES: CNC(=O)COc1cc2cc(Nc3nc(N4CCN(CC5CCN(c6cc(OC)cc7c6CN(C6CCC(=O)NC6=O)C7=O)CC5)CC4)ncc3Cl)ccc2n(C(C)C)c1=O] | 2-{[6-({5-chloro-2-[4-({1-[2-(2,6- dioxopiperidin-3-yl)-6-methoxy-1-oxo- 2,3-dihydro-1H-isoindol-4-yl]piperidin-4- yl}methyl)piperazin-1-yl]pyrimidin-4- yl}amino)-2-oxo-1-(propan-2-yl)-1,2- dihydroquinolin-3-yl]oxy}-N- methylacetamide |
| 120 | 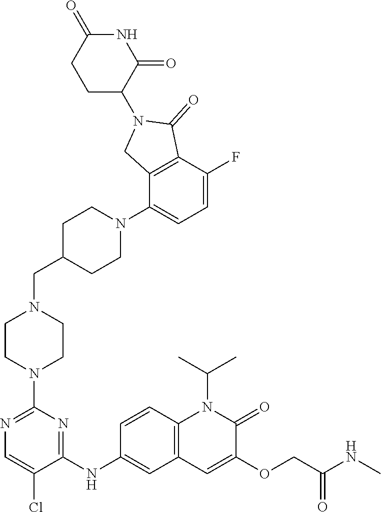 [SMILES: CNC(=O)COc1cc2cc(Nc3nc(N4CCN(CC5CCN(c6ccc(F)c7c6CN(C6CCC(=O)NC6=O)C7=O)CC5)CC4)ncc3Cl)ccc2n(C(C)C)c1=O] | 2-{[6-({5-chloro-2-[4-({1-[2-(2,6- dioxopiperidin-3-yl)-7-fluoro-1-oxo-2,3- dihydro-1H-isoindol-4-yl]piperidin-4- yl}methyl)piperazin-1-yl]pyrimidin-4- yl}amino)-2-oxo-1-(propan-2-yl)-1,2- dihydroquinolin-3-yl]oxy}-N- methylacetamide |
| 121 | 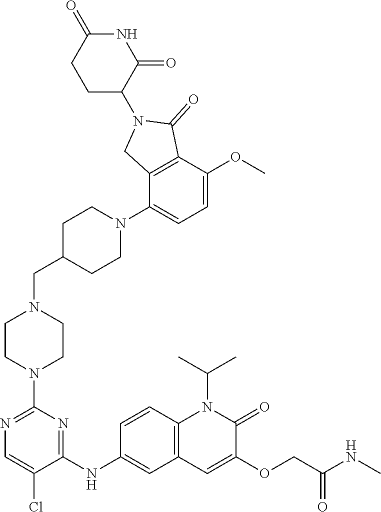 [SMILES: CNC(=O)COc1cc2cc(Nc3nc(N4CCN(CC5CCN(c6ccc(OC)c7c6CN(C6CCC(=O)NC6=O)C7=O)CC5)CC4)ncc3Cl)ccc2n(C(C)C)c1=O] | 2-{[6-({5-chloro-2-[4-({1-[2-(2,6- dioxopiperidin-3-yl)-7-methoxy-1-oxo- 2,3-dihydro-1H-isoindol-4-yl]piperidin-4- yl}methyl)piperazin-1-yl]pyrimidin-4- yl}amino)-2-oxo-1-(propan-2-yl)-1,2- dihydroquinolin-3-yl]oxy}-N- methylacetamide |
| 122 | 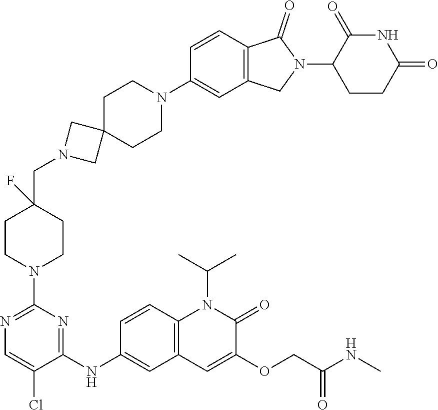 [SMILES: CNC(=O)COc1cc2cc(Nc3nc(N4CCC(F)(CN5CC6(CCN(c7ccc8c(c7)CN(C7CCC(=O)NC7=O)C8=O)CC6)C5)CC4)ncc3Cl)ccc2n(C(C)C)c1=O] | 2-{[6-({5-chloro-2-[4-({7-[2-(2,6- dioxopiperidin-3-yl)-1-oxo-2,3-dihydro- 1H-isoindol-5-yl]-2,7- diazaspiro[3.5]nonan-2-yl}methyl)-4- fluoropiperidin-1-yl]pyrimidin-4- yl}amino)-2-oxo-1-(propan-2-yl)-1,2- dihydroquinolin-3-yl]oxy}-N- methylacetamide |
| 123 | 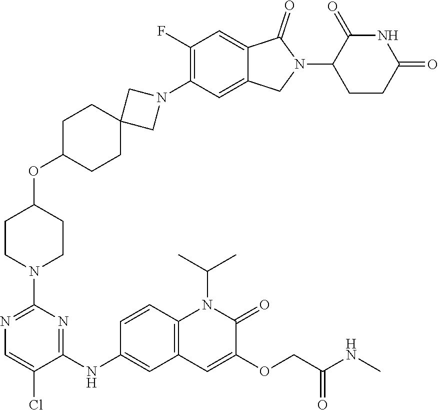 [SMILES: CNC(=O)COc1cc2cc(Nc3nc(N4CCC(OC5CCC6(CC5)CN(c5cc7c(cc5F)C(=O)N(C5CCC(=O)NC5=O)C7)C6)CC4)ncc3Cl)ccc2n(C(C)C)c1=O] | 2-{[6-({5-chloro-2-[4-({2-[2-(2,6- dioxopiperidin-3-yl)-6-fluoro-1-oxo-2,3- dihydro-1H-isoindol-5-yl]-2- azaspiro[3.5]nonan-7-yl}oxy)piperidin-1- yl]pyrimidin-4-yl}amino)-2-oxo-1- (propan-2-yl)-1,2-dihydroquinolin-3- yl]oxy}-N-methylacetamide |
| 124 | 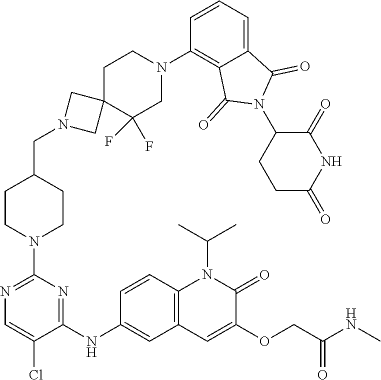 [SMILES: CNC(=O)COc1cc2cc(Nc3nc(N4CCC(CN5CC6(CCN(c7cccc8c7C(=O)N(C7CCC(=O)NC7=O)C8=O)CC6(F)F)C5)CC4)ncc3Cl)ccc2n(C(C)C)c1=O] | 2-{[6-({5-chloro-2-[4-({7-[2-(2,6- dioxopiperidin-3-yl)-1,3-dioxo-2,3- dihydro-1H-isoindol-4-yl]-5,5-difluoro- 2,7-diazaspiro[3.5]nonan-2- yl}methyl)piperidin-1-yl]pyrimidin-4- yl}amino)-2-oxo-1-(propan-2-yl)-1,2- dihydroquinolin-3-yl]oxy}-N- methylacetamide |
| 125 | 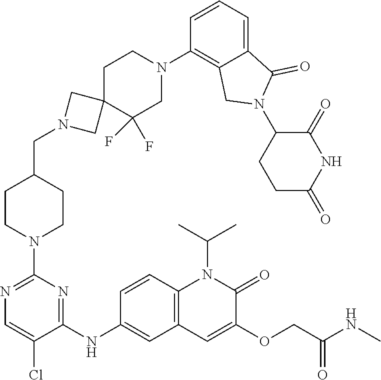 [SMILES: CNC(=O)COc1cc2cc(Nc3nc(N4CCC(CN5CC6(CCN(c7cccc8c7CN(C7CCC(=O)NC7=O)C8=O)CC6(F)F)C5)CC4)ncc3Cl)ccc2n(C(C)C)c1=O] | 2-{[6-({5-chloro-2-[4-({7-[2-(2,6- dioxopiperidin-3-yl)-1-oxo-2,3-dihydro- 1H-isoindol-4-yl]-5,5-difluoro-2,7- diazaspiro[3.5]nonan-2- yl}methyl)piperidin-1-yl]pyrimidin-4- yl}amino)-2-oxo-1-(propan-2-yl)-1,2- dihydroquinolin-3-yl]oxy}-N- methylacetamide |
| 126 | 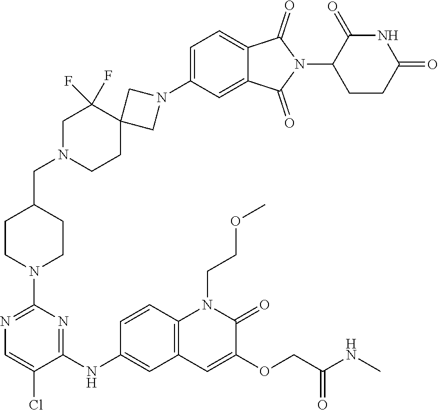 [SMILES: CNC(=O)COc1cc2cc(Nc3nc(N4CCC(CN5CCC6(CN(c7ccc8c(c7)C(=O)N(C7CCC(=O)NC7=O)C8=O)C6)C(F)(F)C5)CC4)ncc3Cl)ccc2n(CCOC)c1=O] | 2-{[6-({5-chloro-2-[4-({2-[2-(2,6- dioxopiperidin-3-yl)-1,3-dioxo-2,3- dihydro-1H-isoindol-5-yl]-5,5-difluroo- 2,7-diazaspiro[3.5]nonan-7- yl}methyl)piperidin-1-yl]pyrimidin-4- yl}amino)-1-(2-methoxyethyl)-2-oxo-1,2- dihydroquinolin-3-yl]oxy}-N- methylacetamide |
| 127 | 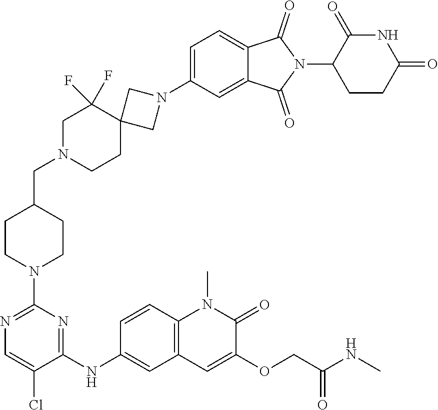 [SMILES: CNC(=O)COc1cc2cc(Nc3nc(N4CCC(CN5CCC6(CN(c7ccc8c(c7)C(=O)N(C7CCC(=O)NC7=O)C8=O)C6)C(F)(F)C5)CC4)ncc3Cl)ccc2n(C)c1=O] | 2-{[6-({5-chloro-2-[4-({2-[2-(2,6- dioxopiperidin-3-yl)-1,3-dioxo-2,3- dihydro-1H-isoindol-5-yl]-5,5-difluroo- 2,7-diazaspiro[3.5]nonan-7- yl}methyl)piperidin-1-yl]pyrimidin-4- yl}amino)-1-methyl-2-oxo-1,2- dihydroquinolin-3-yl]oxy}-N- methylacetamide |
| 128 | 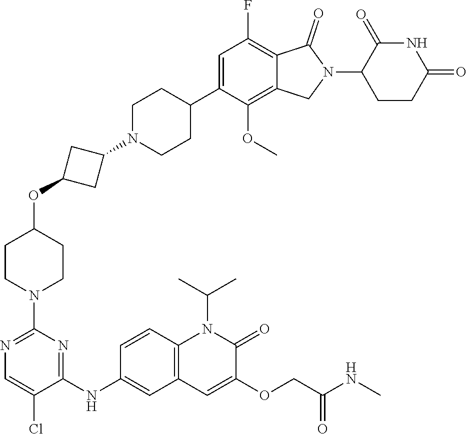 [SMILES: CNC(=O)COc1cc2cc(Nc3nc(N4CCC(O[C@H]5C[C@H](N6CCC(c7cc(F)c8c(c7OC)CN(C7CCC(=O)NC7=O)C8=O)CC6)C5)CC4)ncc3Cl)ccc2n(C(C)C)c1=O] | 2-({6-[(5-chloro-2-{4-[(1r,3r)-3-{4-[2- (2,6-dioxopiperidin-3-yl)-7-fluoro-4- methoxy-1-oxo-2,3-dihydro-1H-isoindol- 5-yl]piperidin-1- yl}cyclobutoxy]piperidin-1-yl}pyrimidin- 4-yl)amino]-2-oxo-1-(propan-2-yl)-1,2- dihydroquinolin-3-yl}oxy)-N- methylacetamide |
| 129 | 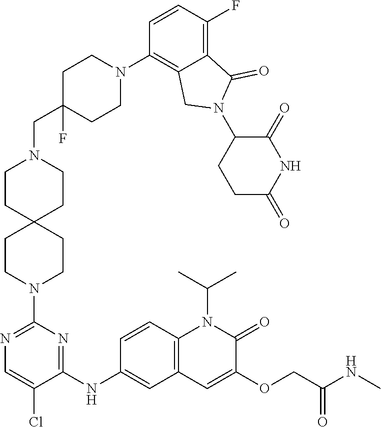 [SMILES: CNC(=O)COc1cc2cc(Nc3nc(N4CCC5(CCN(CC6(F)CCN(c7ccc(F)c8c7CN(C7CCC(=O)NC7=O)C8=O)CC6)CC5)CC4)ncc3Cl)ccc2n(C(C)C)c1=O] | 2-{[6-({5-chloro-2-[9-({1-[2-(2,6- dioxopiperidin-3-yl)-7-fluoro-1-oxo-2,3- dihydro-1H-isoindol-4-yl]-4- fluoropiperidin-4-yl}methyl)-3,9- diazaspiro[5.5]undecna-3-yl]pyrimidin-4- yl}amino)-2-oxo-1-(propan-2-yl)-1,2- dihydroquinolin-3-yl]oxy}-N- methylacetamide |
| 130 | 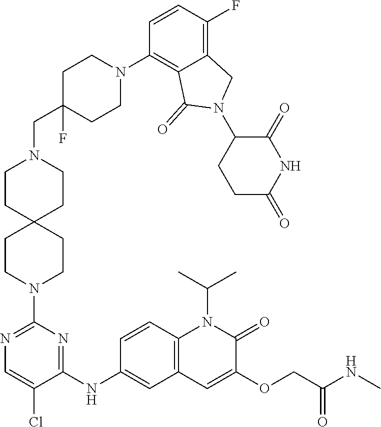 [SMILES: CNC(=O)COc1cc2cc(Nc3nc(N4CCC5(CCN(CC6(F)CCN(c7ccc(F)c8c7C(=O)N(C7CCC(=O)NC7=O)C8)CC6)CC5)CC4)ncc3Cl)ccc2n(C(C)C)c1=O] | 2-{[6-({5-chloro-2-[9-({1-[2-(2,6- dioxopiperidin-3-yl)-7-fluoro-3-oxo-2,3- dihydro-1H-isoindol-4-yl]-4- fluoropiperidin-4-yl}methyl)-3,9- diazaspiro[5.5]undecan-3-yl]pyrimdiin-4- yl}amino)-2-oxo-1-(propan-2-yl)-1,2- dihydroquinolin-3-yl]oxy}-N- methylacetamide |
| 131 | 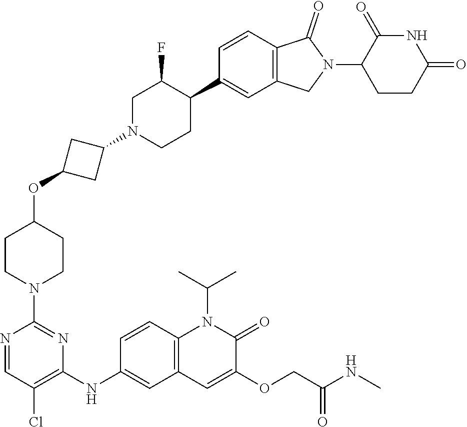 [SMILES: CNC(=O)COc1cc2cc(Nc3nc(N4CCC(O[C@H]5C[C@H](N6CC[C@H](c7ccc8c(c7)CN(C7CCC(=O)NC7=O)C8=O)[C@H](F)C6)C5)CC4)ncc3Cl)ccc2n(C(C)C)c1=O] | 2-({6-[(5-chloro-2-{4-[(1r,3r)-3-[(3S,4R)- 4-[2-(2,6-dioxopiperidin-3-yl)-1-oxo-2,3- dihydro-1H-isoindol-5-yl]-3- fluoropiperidin-1- yl]cyclobutoxy]piperidin-1-yl}pyrimidin- 4-yl)amino]-2-oxo-1-(propan-2-yl)-1,2- dihydroquinolin-3-yl}oxy)-N- methylacetamide |
| 132 | 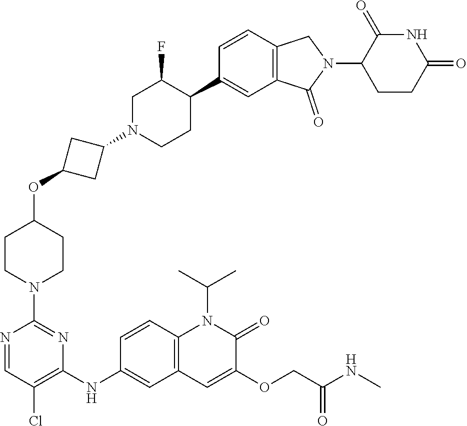 [SMILES: CNC(=O)COc1cc2cc(Nc3nc(N4CCC(O[C@H]5C[C@H](N6CC[C@H](c7ccc8c(c7)C(=O)N(C7CCC(=O)NC7=O)C8)[C@H](F)C6)C5)CC4)ncc3Cl)ccc2n(C(C)C)c1=O] | 2-({6-[(5-chlroo-2-{4-[(1r,3r)-3-[(3S,4R)- 4-[2-(2,6-dioxopiperidin-3-yl)-3-oxo-2,3- dihydro-1H-isoindol-5-yl]-3- fluoropiperidin-1- yl]cyclobutoxy]piperidin-1-yl}pyrimidin- 4-yl)amino]-2-oxo-1-(propan-2-yl)-1,2- dihydroquinolin-3-yl}oxy)-N- methylacetamide |
| 133 | 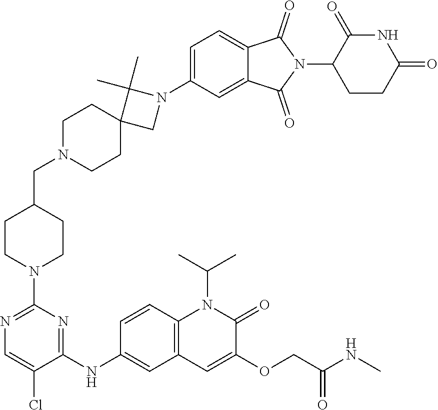 [SMILES: CNC(=O)COc1cc2cc(Nc3nc(N4CCC(CN5CCC6(CC5)CN(c5ccc7c(c5)C(=O)N(C5CCC(=O)NC5=O)C7=O)C6(C)C)CC4)ncc3Cl)ccc2n(C(C)C)c1=O] | 2-{[6-({5-chloro-2-[4-({2-[2-(2,6- dioxopiperidin-3-yl)-1,3-dioxo-2,3- dihydro-1H-isoindol-5-yl]-1,1-dimethyl- 2,7-diazaspiro[3.5]nonan-7- yl}methyl)piperidin-1-yl]pyrimidin-4- yl}amino)-2-oxo-1-(propan-2-yl)-1,2- dihydroquinolin-3-yl]oxy}-N- methylacetamide |
| 134 | 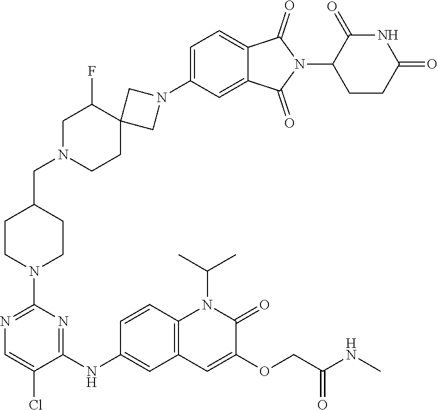 [SMILES: CNC(=O)COc1cc2cc(Nc3nc(N4CCC(CN5CCC6(CN(c7ccc8c(c7)C(=O)N(C7CCC(=O)NC7=O)C8=O)C6)C(F)C5)CC4)ncc3Cl)ccc2n(C(C)C)c1=O] | 2-{[6-({5-chloro-2-[4-({2-[2-(2,6- dioxopiperidin-3-yl)-1,3-dioxo-2,3- dihydro-1H-isoindol-5-yl]-5-fluoro-2,7- diazaspiro[3.5]nonan-7- yl}methyl)piperidin-1-yl]pyrimidin-4- yl}amino)-2-oxo-1-(propan-2-yl)-1,2- dihydroquinolin-3-yl]oxy}-N- methylacetamide |
| 135 | 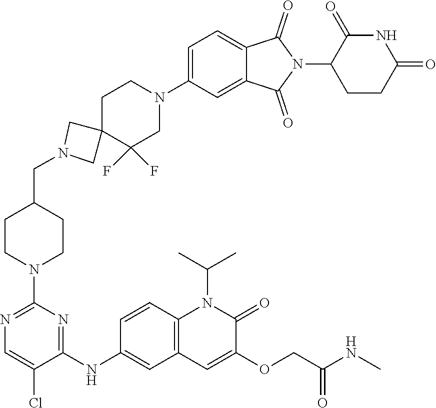 [SMILES: CNC(=O)COc1cc2cc(Nc3nc(N4CCC(CN5CC6(CCN(c7ccc8c(c7)C(=O)N(C7CCC(=O)NC7=O)C8=O)CC6(F)F)C5)CC4)ncc3Cl)ccc2n(C(C)C)c1=O] | 2-{[6-({5-chloro-2-[4-({7-[2-(2,6- dioxopiperidin-3-yl)-1,3-dioxo-2,3- dihydro-1H-isoindol-5-yl]-5,5-difluoro- 2,7-diazaspiro[3.5]nonan-2- yl}methyl)piperidin-1-yl]pyrimidin-4- yl}amino)-2-oxo-1-(propan-2-yl)-1,2- dihydroquinolin-3-yl]oxy}-N- methylacetamide |
| 136 | 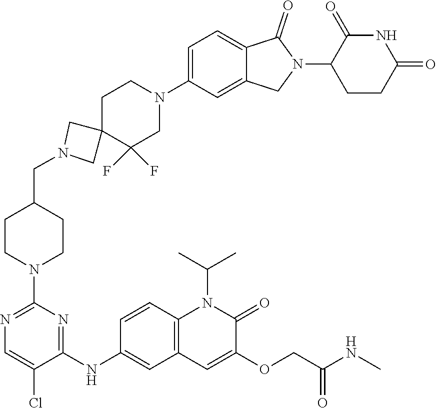 [SMILES: CNC(=O)COc1cc2cc(Nc3nc(N4CCC(CN5CC6(CCN(c7ccc8c(c7)CN(C7CCC(=O)NC7=O)C8=O)CC6(F)F)C5)CC4)ncc3Cl)ccc2n(C(C)C)c1=O] | 2-{[6-({5-chloro-2-[4-({7-[2-(2,6- dioxopiperidin-3-yl)-1-oxo-2,3-dihydro- 1H-isoindol-5-yl]-5,5-difluoro-2,7- diazaspiro[3.5]nonan-2- yl}methyl)piperidin-1-yl]pyrimidin-4- yl}amino)-2-oxo-1-(propan-2-yl)-1,2- dihydroquinolin-3-yl]oxy}-N- methylacetamide |
| 137 | 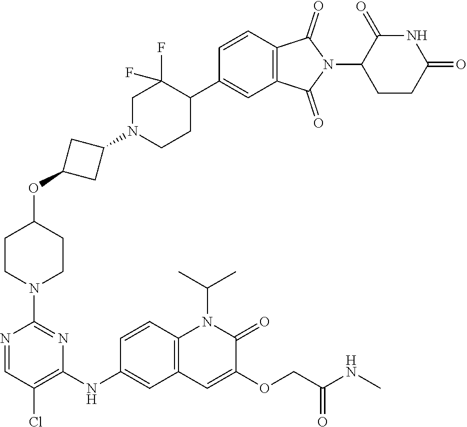 [SMILES: CNC(=O)COc1cc2cc(Nc3nc(N4CCC(O[C@H]5C[C@H](N6CCC(c7ccc8c(c7)C(=O)N(C7CCC(=O)NC7=O)C8=O)C(F)(F)C6)C5)CC4)ncc3Cl)ccc2n(C(C)C)c1=O] | 2-({6-[(5-chloro-2-{4-[(1r,3r)-3-{4-[2- (2,6-dioxopiperidin-3-yl)-1,3-dioxo-2,3- dihydro-1H-isoindol-5-yl]-3,3- difluoropiperiidn-1- yl}cyclobutoxy]piperidin-1-yl}pyrimidin- 4-yl)amino]-2-oxo-1-(propan-2-yl)-1,2- dihydroquinolin-3-yl}oxy)-N- methylacetamide |
| 138 | 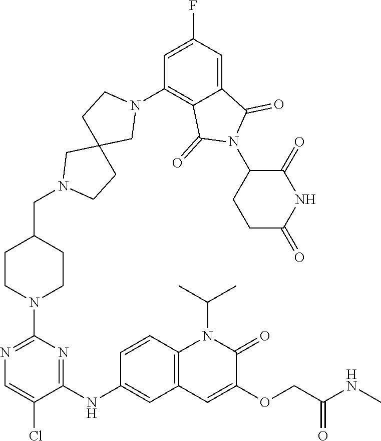 [SMILES: CNC(=O)COc1cc2cc(Nc3nc(N4CCC(CN5CCC6(CCN(c7cc(F)cc8c7C(=O)N(C7CCC(=O)NC7=O)C8=O)C6)C5)CC4)ncc3Cl)ccc2n(C(C)C)c1=O] | 2-{[6-({5-chloro-2-[4-({7-[2-(2,6- dioxopiperidin-3-yl)-6-fluoro-1,3-dioxo- 2,3-dihydro-1H-isoindol-4-yl]-2,7- diazaspiro[4.4]nonan-2- yl}methyl)piperidiin-1-yl]pyrimidin-4- yl}amino)-2-oxo-1-(propan-2-yl)-1,2- dihydroquinolin-3-yl]oxy}-N- methylacetamide |
| 139 | 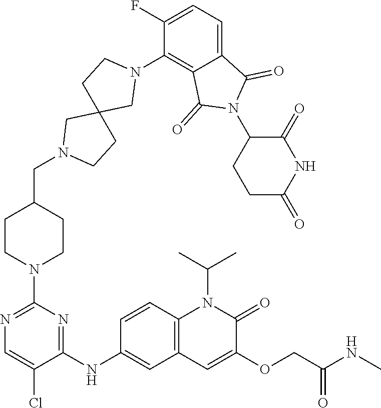 [SMILES: CNC(=O)COc1cc2cc(Nc3nc(N4CCC(CN5CCC6(CCN(c7c(F)ccc8c7C(=O)N(C7CCC(=O)NC7=O)C8=O)C6)C5)CC4)ncc3Cl)ccc2n(C(C)C)c1=O] | 2-{[6-({5-chloro-2-[4-({7-[2-(2,6- dioxopiperidin-3-yl)-5-fluoro-1,3-dioxo- 2,3-dihydro-1H-isoindol-4-yl]-2,7- diazaspiro[4.4]nonan-2- yl}methyl)piperidin-1-yl]pyrimidin-4- yl}amino)-2-oxo-1-(propan-2-yl)-1,2- dihydroquinolin-3-yl]oxy}-N- methylacetamide |
| 140 | 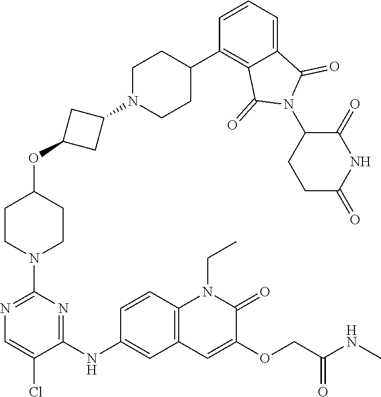 [SMILES: CCn1c(=O)c(OCC(=O)NC)cc2cc(Nc3nc(N4CCC(O[C@H]5C[C@H](N6CCC(c7cccc8c7C(=O)N(C7CCC(=O)NC7=O)C8=O)CC6)C5)CC4)ncc3Cl)ccc21] | 2-({6-[(5-chloro-2-{4-[(1r,3r)-3-{4-[2- (2,6-dioxopiperidin-3-yl)-1,3-dioxo-2,3- dihydro-1H-isoindol-4-yl]piperidin-1- yl}cyclobutoxy]piperidin-1-yl}pyrimidin- 4-yl)amino]-1-ethyl-2-oxo-1,2- dihydroquinolin-3-yl}oxy)-N- methylacetamide |
| 141 | 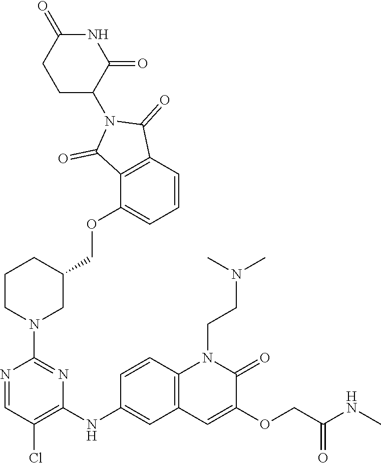 [SMILES: CNC(=O)COc1cc2cc(Nc3nc(N4CCC[C@H](COc5cccc6c5C(=O)N(C5CCC(=O)NC5=O)C6=O)C4)ncc3Cl)ccc2n(CCN(C)C)c1=O] | 2-{[6-({5-chloro-2-[(3S)-3-({[2-(2,6- dioxopiperidin-3-yl)-1,3-dioxo-2,3- dihydro-1H-isoindol-4- yl]oxy}methyl)piperidin-1-yl]pyrimidin- 4-yl}amino)-1-[2-(dimethylamino)ethyl]- 2-oxo-1,2-dihydroquinolin-3-yl]oxy}-N- methylacetamide |
| 142 | 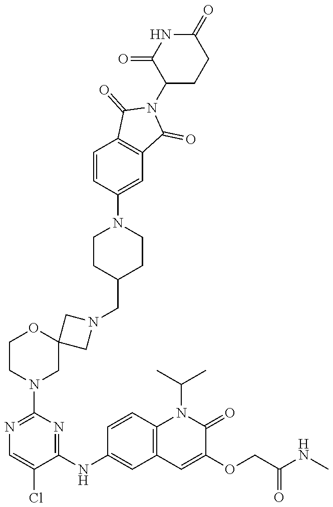 [SMILES: CNC(=O)COc1cc2cc(Nc3nc(N4CCOC5(CN(CC6CCN(c7ccc8c(c7)C(=O)N(C7CCC(=O)NC7=O)C8=O)CC6)C5)C4)ncc3Cl)ccc2n(C(C)C)c1=O] | 2-{[6-({5-chloro-2-[2-({1-[2-(2,6- dioxopiperidin-3-yl)-1,3-dioxo-2,3- dihydro-1H-isoindol-5-yl]piperidin-4- yl}methyl)-5-oxa-2,8- diazaspiro[3.5]nonan-8-yl]pyrimidin-4- yl}amino)-2-oxo-1-(propan-2-yl)-1,2- dihydroquinolin-3-yl]oxy}-N- methylacetamide |
| 143 | 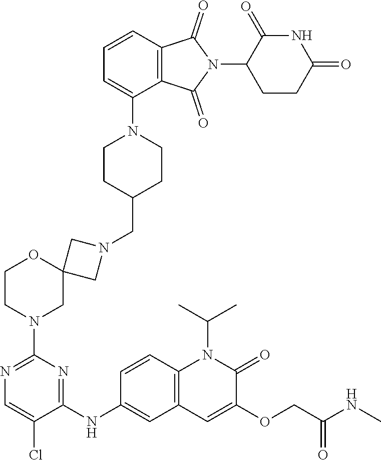 [SMILES: CNC(=O)COc1cc2cc(Nc3nc(N4CCOC5(CN(CC6CCN(c7cccc8c7C(=O)N(C7CCC(=O)NC7=O)C8=O)CC6)C5)C4)ncc3Cl)ccc2n(C(C)C)c1=O] | 2-{[6-({5-chloro-2-[2-({1-[2-(2,6- dioxopiperidin-3-yl)-1,3-dioxo-2,3- dihydro-1H-isoindol-4-yl]piperidin-4- yl}methyl)-5-oxa-2,8- diazaspiro[3.5]nonan-8-yl]pyrimidin-4- yl}amino)-2-oxo-1-(propan-2-yl)-1,2- dihydroquinolin-3-yl]oxy}-N- methylacetamide |
| 144 | 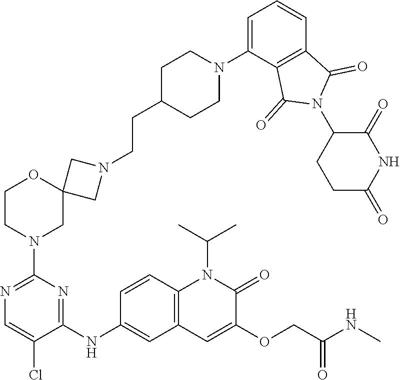 [SMILES: CNC(=O)COc1cc2cc(Nc3nc(N4CCOC5(CN(CCC6CCN(c7cccc8c7C(=O)N(C7CCC(=O)NC7=O)C8=O)CC6)C5)C4)ncc3Cl)ccc2n(C(C)C)c1=O] | 2-{[6-({5-chloro-2-[2-(2-{1-[2-(2,6- dioxopiperidin-3-yl)-1,3-dioxo-2,3- dihydro-1H-isoindol-4-yl]piperidin-4- yl}ethyl)-5-oxa-2,8-diazaspiro[3.5]nonan- 8-yl]pyrimidin-4-yl}amino)-2-oxo-1- (propan-2-yl)-1,2-dihydroquinolin-3- yl]oxy}-N-methylacetamide |
| 145 | 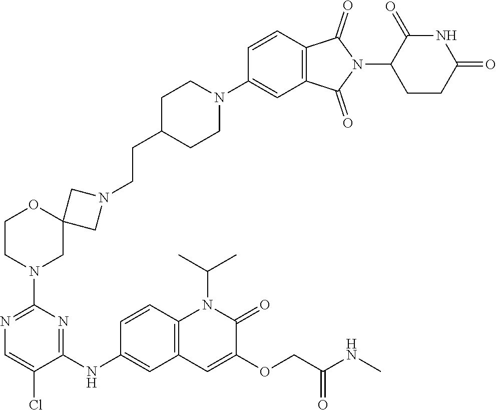 [SMILES: CNC(=O)COc1cc2cc(Nc3nc(N4CCOC5(CN(CCC6CCN(c7ccc8c(c7)C(=O)N(C7CCC(=O)NC7=O)C8=O)CC6)C5)C4)ncc3Cl)ccc2n(C(C)C)c1=O] | 2-{[6-({5-chloro-2-[2-(2-{1-[2-(2,6- dioxopiperidin-3-yl)-1,3-dioxo-2,3- dihydro-1H-isoindol-5-yl]piperidin-4- yl}ethyl)-5-oxa-2,8-diazaspiro[3.5]nonan- 8-yl]pyrimidin-4-yl}amino)-2-oxo-1- (propan-2-yl)-1,2-dihydroquinolin-3- yl]oxy}-N-methylacetamide |
| 146 | 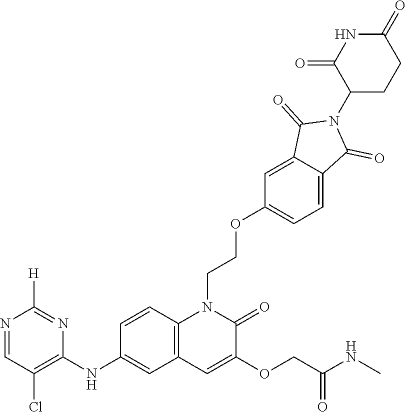 [SMILES: CNC(=O)COc1cc2cc(Nc3ncncc3Cl)ccc2n(CCOc2ccc3c(c2)C(=O)N(C2CCC(=O)NC2=O)C3=O)c1=O] | 2-({6-[(5-chloropyrimidin-4-yl)amino]-1- (2-{[2-(2,6-dioxopiperidin-3-yl)-1,3- dioxo-2,3-dihydro-1H-isoindol-5- yl]oxy}ethyl)-2-oxo-1,2-dihydroquinolin- 3-yl}oxy)-N-methylacetamide |
| 147 | 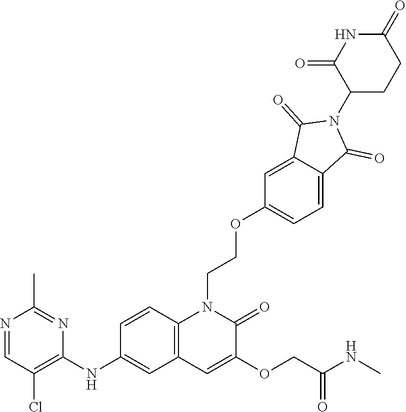 [SMILES: CNC(=O)COc1cc2cc(Nc3nc(C)ncc3Cl)ccc2n(CCOc2ccc3c(c2)C(=O)N(C2CCC(=O)NC2=O)C3=O)c1=O] | 2-({6-[(5-chloro-2-methylpyrimidin-4- yl)amino]-1-(2-{[2-(2,6-dioxopiperidin-3- yl)-1,3-dioxo-2,3-dihydro-1H-isoindol-5- yl]oxy}ethyl)-2-oxo-1,2-dihydroquinolin- 3-yl}oxy)-N-methylacetamide |
| 148 | 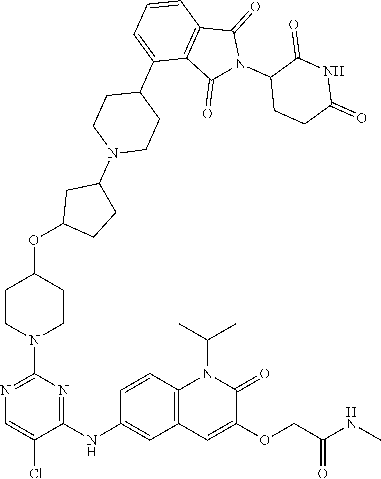 [SMILES: CNC(=O)COc1cc2cc(Nc3nc(N4CCC(OC5CCC(N6CCC(c7cccc8c7C(=O)N(C7CCC(=O)NC7=O)C8=O)CC6)C5)CC4)ncc3Cl)ccc2n(C(C)C)c1=O] | 2-({6-[(5-chloro-2-{4-[(3-{4-[2-(2,6- dioxopiperidin-3-yl)-1,3-dioxo-2,3- dihydro-1H-isoindol-4-yl]piperidin-1- yl}cyclopentyl)oxy]piperidin-1- yl}pyrimidin-4-yl)amino]-2-oxo-1- (propan-2-yl)-1,2-dihydroquinolin-3- yl}oxy)-N-methylacetamide |
| 149 | 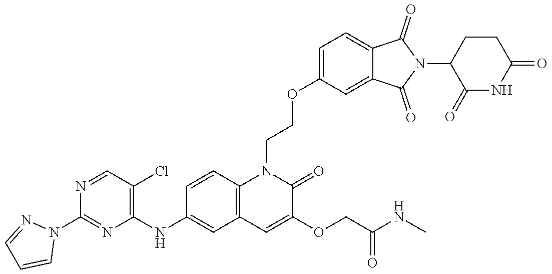 [SMILES: CNC(=O)COc1cc2cc(Nc3nc(-n4cccn4)ncc3Cl)ccc2n(CCOc2ccc3c(c2)C(=O)N(C2CCC(=O)NC2=O)C3=O)c1=O] | 2-[({[5-chloro-2-(1H-pyrazol-1- yl)pyrimidin-4-yl]amino}-1-(2-{[2-(2,6- dioxopiperidin-3-yl)-1,3-dioxo-2,3- dihydro-1H-isoindol-5-yl]oxy}ethyl)-2- oxo-1,2-dihydroquinolin-3-yl)oxy]-N- methylacetamide |
| 150 | 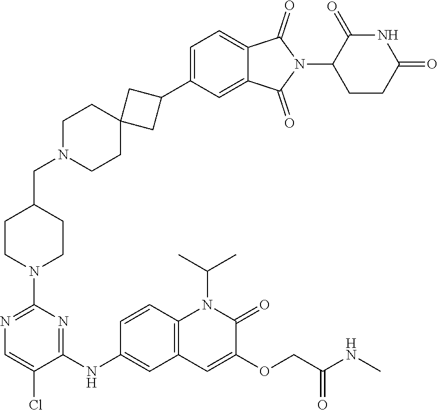 [SMILES: CNC(=O)COc1cc2cc(Nc3nc(N4CCC(CN5CCC6(CC5)CC(c5ccc7c(c5)C(=O)N(C5CCC(=O)NC5=O)C7=O)C6)CC4)ncc3Cl)ccc2n(C(C)C)c1=O] | 2-{[6-({5-chloro-2-[4-({2-[2-(2,6- dioxopiperidin-3-yl)-1,3-dioxo-2,3- dihydro-1H-isoindol-5-yl]-7- azaspiro[3.5]nonan-7- yl}methyl)piperidin-1-yl]pyrimidin-4- yl}amino)-2-oxo-1-(propan-2-yl)-1,2- dihydroquinolin-3-yl]oxy}-N- methylacetamide |
| 151 | 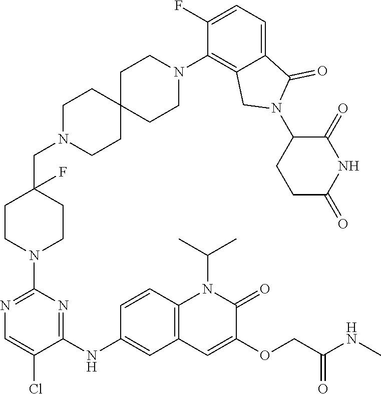 [SMILES: CNC(=O)COc1cc2cc(Nc3nc(N4CCC(F)(CN5CCC6(CC5)CCN(c5c(F)ccc7c5CN(C5CCC(=O)NC5=O)C7=O)CC6)CC4)ncc3Cl)ccc2n(C(C)C)c1=O] | 2-{[6-({5-chloro-2-[4-({9-[2-(2,6- dioxopiperiidn-3-yl)-5-fluoro-1-oxo-2,3- dihydro-1H-isoindol-4-yl]-3,9- diazaspiro[5.5]undecan-3-yl}methyl)-4- fluropiperidin-1-yl]pyrimidin-4- yl}amino)-2-oxo-1-(propan-2-yl)-1,2- dihydroquinolin-3-yl]oxy}-N- methylacetamide |
| 152 | 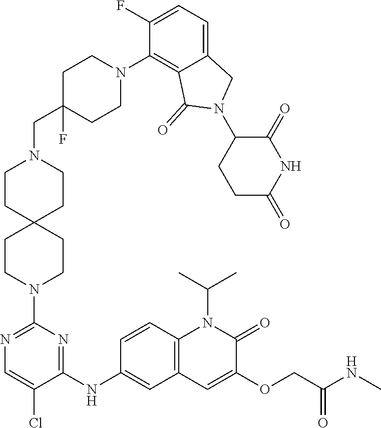 [SMILES: CNC(=O)COc1cc2cc(Nc3nc(N4CCC5(CCN(CC6(F)CCN(c7c(F)ccc8c7C(=O)N(C7CCC(=O)NC7=O)C8)CC6)CC5)CC4)ncc3Cl)ccc2n(C(C)C)c1=O] | 2-{[6-({5-chloro-2-[9-({1-[2-(2,6- dioxopiperidin-3-yl)-5-fluoro-3-oxo-2,3- dihydro-1H-isoindol-4-yl]-4- fluoropiperidin-4-yl}methyl)-3,9- diazaspior[5.5]undecan-3-yl]pyrimidin-4- yl}amino)-2-oxo-1-(propan-2-yl)-1,2- dihydroquinolin-3-yl]oxy}-N- methylacetamide |
| 153 | 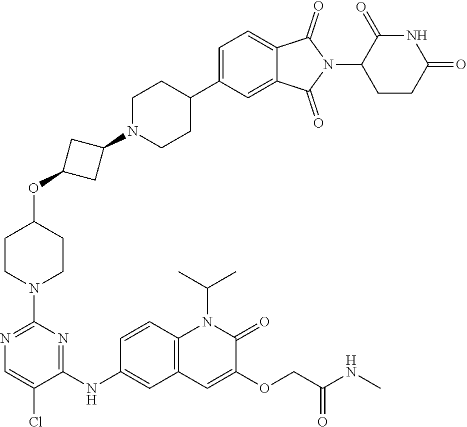 [SMILES: CNC(=O)COc1cc2cc(Nc3nc(N4CCC(O[C@H]5C[C@@H](N6CCC(c7ccc8c(c7)C(=O)N(C7CCC(=O)NC7=O)C8=O)CC6)C5)CC4)ncc3Cl)ccc2n(C(C)C)c1=O] | 2-({6-[(5-chloro-2-{4-[(1s,3s)-3-{4-[2- (2,6-dioxopiperidin-3-yl)-1,3-dioxo-2,3- dihydro-1H-isoindol-5-yl]piperidin-1- yl}cyclobutoxy]piperidin-1-yl}pyrimidin- 4-yl)amino]-2-oxo-1-(propan-2-yl)-1,2- dihydroquinolin-3-yl}oxy)-N- methylacetamide |
| 154 | 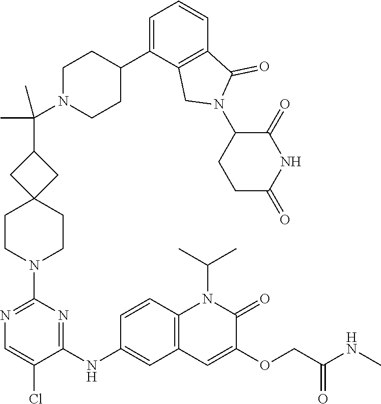 [SMILES: CNC(=O)COc1cc2cc(Nc3nc(N4CCC5(CC4)CC(C(C)(C)N4CCC(c6cccc7c6CN(C6CCC(=O)NC6=O)C7=O)CC4)C5)ncc3Cl)ccc2n(C(C)C)c1=O] | 2-{[6-({5-chloro-2-[2-(2-{4-[2-(2,6- dioxopiperidin-3-yl)-1-oxo-2,3-dihydro- 1H-isoindol-4-yl]piperidin-1-yl}propan-2- yl)-7-azaspiro[3.5]nonan-7-yl]pyrimidin- 4-yl}amino)-2-oxo-1-(propan-2-yl)-1,2- dihydroquinolin-3-yl]oxy}-N- methylacetamide |
| 155 | 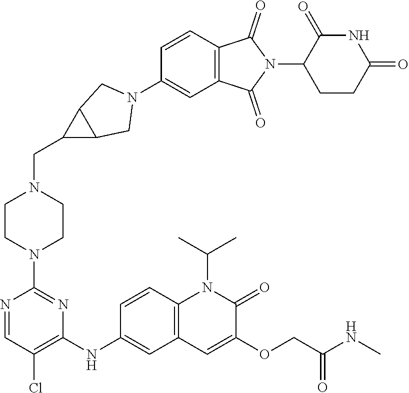 [SMILES: CNC(=O)COc1cc2cc(Nc3nc(N4CCN(CC5C6CN(c7ccc8c(c7)C(=O)N(C7CCC(=O)NC7=O)C8=O)CC56)CC4)ncc3Cl)ccc2n(C(C)C)c1=O] | 2-{[6-({5-chloro-2-[4-({3-[2-(2,6- dioxopiperidin-3-yl)-1,3-dioxo-2,3- dihydro-1H-isoindol-5-yl]-3- azabicyclo[3.1.0]hexan-6- yl}methyl)piperazin-1-yl]pyrimidin-4- yl}amino)-2-oxo-1-(propan-2-yl)-1,2- dihydroquinolin-3-yl]oxy}-N- methylacetamide |
| 156 | 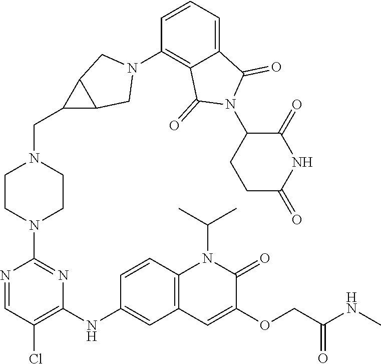 [SMILES: CNC(=O)COc1cc2cc(Nc3nc(N4CCN(CC5C6CN(c7cccc8c7C(=O)N(C7CCC(=O)NC7=O)C8=O)CC56)CC4)ncc3Cl)ccc2n(C(C)C)c1=O] | 2-{[6-({5-chloro-2-[4-({3-[2-(2,6- dioxopiperidin-3-yl)-1,3-dioxo-2,3- dihydro-1H-isoindol-4-yl]-3- azabicyclo[3.1.0]hexan-6- yl}methyl)piperazin-1-yl]pyrimidin-4- yl}amino)-2-oxo-1-(propan-2-yl)-1,2- dihydroquinolin-3-yl]oxy}-N- methylacetamide |
| 157 | 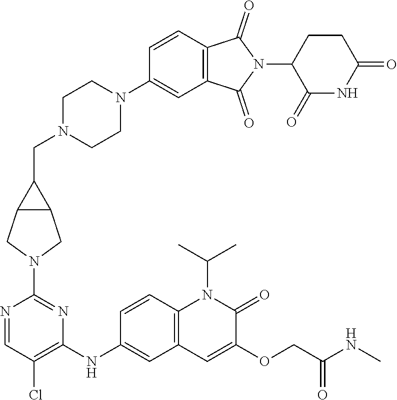 [SMILES: CNC(=O)COc1cc2cc(Nc3nc(N4CC5C(CN6CCN(c7ccc8c(c7)C(=O)N(C7CCC(=O)NC7=O)C8=O)CC6)C5C4)ncc3Cl)ccc2n(C(C)C)c1=O] | 2-{[6-({5-chloro-2-[6-({4-[2-(2,6- dioxopiperidin-3-yl)-1,3-dioxo-2,3- dihydro-1H-isoindol-5-yl]piperazin-1- yl}methyl)-3-azabicyclo[3.1.0]hexan-3- yl]pyrimidin-4-yl}amino)-2-oxo-1- (propan-2-yl)-1,2-dihydroquinolin-3- yl]oxy}-N-methylacetamide |
| 158 | 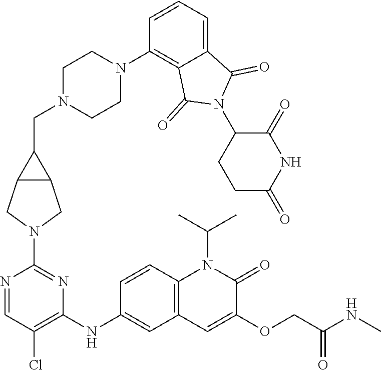 [SMILES: CNC(=O)COc1cc2cc(Nc3nc(N4CC5C(CN6CCN(c7cccc8c7C(=O)N(C7CCC(=O)NC7=O)C8=O)CC6)C5C4)ncc3Cl)ccc2n(C(C)C)c1=O] | 2-{[6-({5-chloro-2-[6-({4-[2-(2,6- dioxopiperidin-3-yl)-1,3-dioxo-2,3- dihydro-1H-isoindol-4-yl]piperazin-1- yl}methyl)-3-azabicyclo[3.1.0]hexan-3- yl]pyrimidin-4-yl}amino)-2-oxo-1- (propan-2-yl)-1,2-dihydroquinolin-3- yl]oxy}-N-methylacetamide |
| 159 | 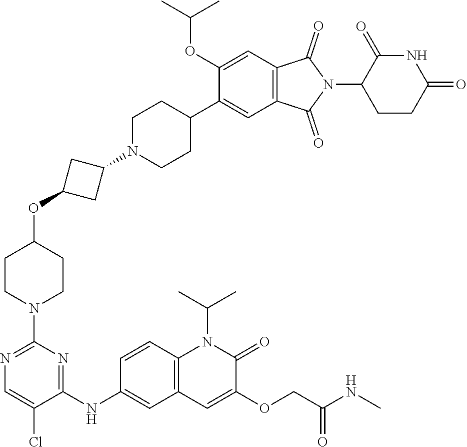 [SMILES: CNC(=O)COc1cc2cc(Nc3nc(N4CCC(O[C@H]5C[C@H](N6CCC(c7cc8c(cc7OC(C)C)C(=O)N(C7CCC(=O)NC7=O)C8=O)CC6)C5)CC4)ncc3Cl)ccc2n(C(C)C)c1=O] | 2-({6-[(5-chloro-2-{4-[(1r,3r)-3-{4-[2- (2,6-dioxopiperidin-3-yl)-1,3-dioxo-6- (propan-2-yloxy)-2,3-dihydro-1H- isoindol-5-yl]piperidin-1- yl}cyclobutoxy]piperidin-1-yl}pyrimidin- 4-yl)amino]-2-oxo-1-(propan-2-yl)-1,2- dihydroquinolin-3-yl}oxy)-N- methylacetamide |
| 160 | 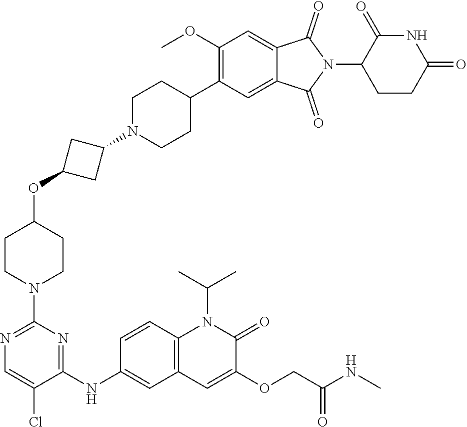 [SMILES: CNC(=O)COc1cc2cc(Nc3nc(N4CCC(O[C@H]5C[C@H](N6CCC(c7cc8c(cc7OC)C(=O)N(C7CCC(=O)NC7=O)C8=O)CC6)C5)CC4)ncc3Cl)ccc2n(C(C)C)c1=O] | 2-({6-[(5-chloro-2-{4-[(1r,3r)-3-{4-[2- (2,6-dioxopiperidin-3-yl)-6-methoxy-1,3- dioxo-2,3-dihydro-1H-isoindol-5- yl]piperidin-1-yl}cyclobutoxy]piperidin- 1-yl}pyrimidin-4-yl)amino]-2-oxo-1- (propan-2-yl)-1,2-dihydroquinolin-3- yl}oxy)-N-methylacetamide |
| 161 | 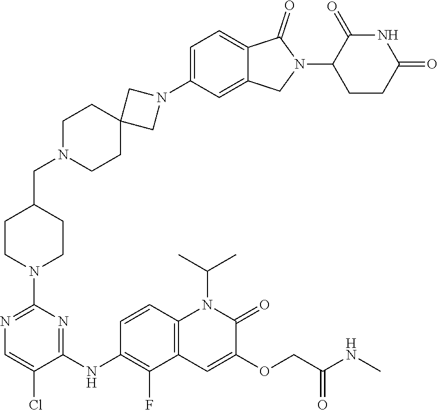 [SMILES: CNC(=O)COc1cc2c(F)c(Nc3nc(N4CCC(CN5CCC6(CC5)CN(c5ccc7c(c5)CN(C5CCC(=O)NC5=O)C7=O)C6)CC4)ncc3Cl)ccc2n(C(C)C)c1=O] | 2-{[6-({5-chloro-2-[4-({2-[2-(2,6- dioxopiperidin-3-yl)-1-oxo-2,3-dihydro- 1H-isoindol-5-yl]-2,7- diazaspiro[3.5]nonan-7- yl}methyl)pyiperidin-1-yl]pyrimdiin-4- yl}amino)-5-fluoro-2-oxo-1-(propan-2- yl)-1,2-dihydroquinolin-3-yl]oxy}-N- methylacetamide |
| 162 | 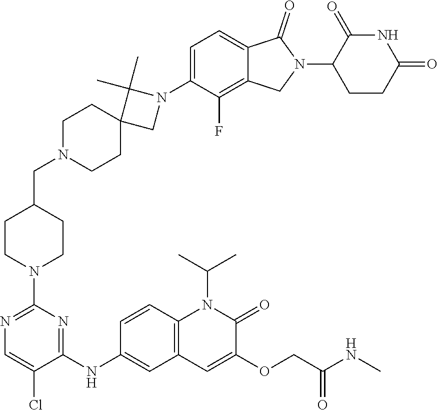 [SMILES: CNC(=O)COc1cc2cc(Nc3nc(N4CCC(CN5CCC6(CC5)CN(c5ccc7c(c5F)CN(C5CCC(=O)NC5=O)C7=O)C6(C)C)CC4)ncc3Cl)ccc2n(C(C)C)c1=O] | 2-{[6-({5-chloro-2-[4-({2-[2-(2,6- dioxopiperidin-3-yl)-4-fluoro-1-oxo-2,3- dihydro-1H-isoindol-5-yl]-1,1-dimethyl- 2,7-diazaspiro[3.5]nonan-7- yl}methyl)piperidin-1-yl]pyrimidin-4- yl}amino)-2-oxo-1-(propna-2-yl)-1,2- dihydroquinolin-3-yl]oxy}-N- methylacetamide |
| 163 | 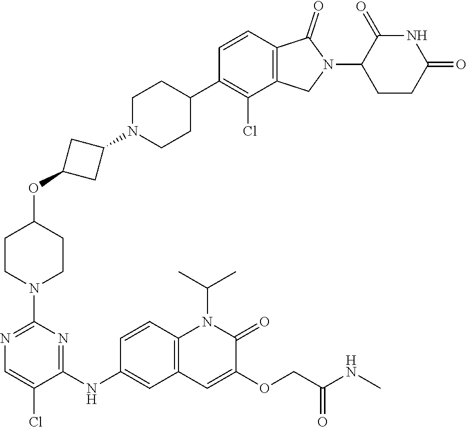 [SMILES: CNC(=O)COc1cc2cc(Nc3nc(N4CCC(O[C@H]5C[C@H](N6CCC(c7ccc8c(c7Cl)CN(C7CCC(=O)NC7=O)C8=O)CC6)C5)CC4)ncc3Cl)ccc2n(C(C)C)c1=O] | 2-({6-[(5-chloro-2-{4-[(1r,3r)-3-{4-[4- chloro-2-(2,6-dioxopiperidin-3-yl)-1-oxo- 2,3-dihydro-1H-isoindol-5-yl]piperidin-1- yl}cyclobutoxy]piperidin-1-yl}pyrimidin- 4-yl)amino]-2-oxo-1-(propan-2-yl)-1,2- dihydroquinolin-3-yl}oxy)-N- methylacetamide |
| 164 | 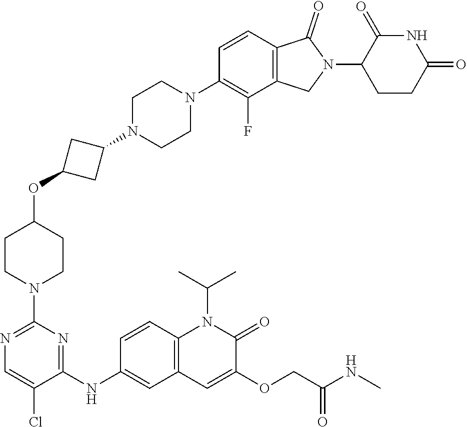 [SMILES: CNC(=O)COc1cc2cc(Nc3nc(N4CCC(O[C@H]5C[C@H](N6CCN(c7ccc8c(c7F)CN(C7CCC(=O)NC7=O)C8=O)CC6)C5)CC4)ncc3Cl)ccc2n(C(C)C)c1=O] | 2-({6-[(5-chloro-2-{4-[(1r,3r)-3-{4-[2- (2,6-dioxopiperidin-3-yl)-4-fluoro-1-oxo- 2,3-dihydro-1H-isoindol-5-yl]piperazin-1- yl}cyclobutoxy]piperidin-1-yl}pyrimidin- 4-yl)amino]-2-oxo-1-(propan-2-yl)-1,2- dihydroquinolin-3-yl}oxy)-N- methylacetamide |
| 165 | 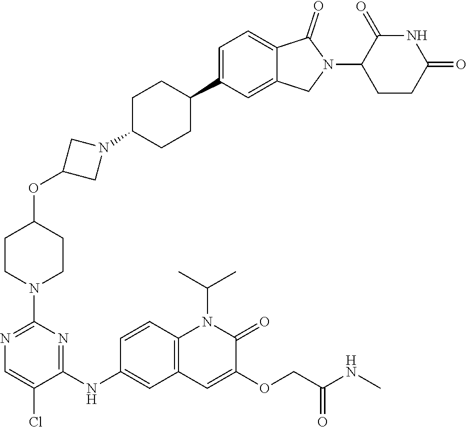 [SMILES: CNC(=O)COc1cc2cc(Nc3nc(N4CCC(OC5CN([C@H]6CC[C@H](c7ccc8c(c7)CN(C7CCC(=O)NC7=O)C8=O)CC6)C5)CC4)ncc3Cl)ccc2n(C(C)C)c1=O] | 2-{[6-({5-chloro-2-[4-({1-[(1r,4r)-4-[2- (2,6-dioxopiperidin-3-yl)-1-oxo-2,3- dihydro-1H-isoindol-5- yl]cyclohexyl]azetidin-3- yl}oxy)piperidin-1-yl]pyrimidin-4- yl}amino)-2-oxo-1-(propan-2-yl)-1,2- dihydroquinolin-3-yl]oxy}-N- methylacetamide |
| 166 | 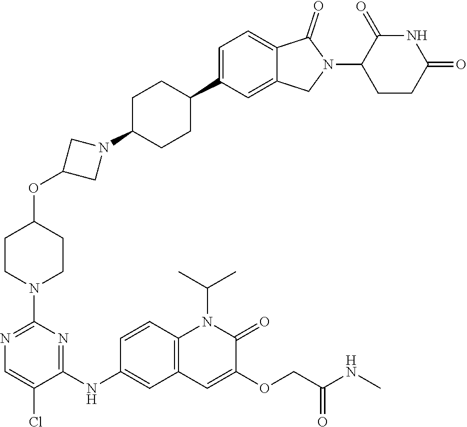 [SMILES: CNC(=O)COc1cc2cc(Nc3nc(N4CCC(OC5CN([C@H]6CC[C@@H](c7ccc8c(c7)CN(C7CCC(=O)NC7=O)C8=O)CC6)C5)CC4)ncc3Cl)ccc2n(C(C)C)c1=O] | 2-{[6-({5-chloro-2-[4-({1-[(1s,4s)-4-[2- (2,6-dioxopiperidin-3-yl)-1-oxo-2,3- dihydro-1H-isoindol-5- yl]cyclohexyl]azetidin-3- yl}oxy)piperidin-1-yl]pyrimidin-4- yl}amino)-2-oxo-1-(propan-2-yl)-1,2- dihydroquinolin-3-yl]oxy}-N- methylacetamide |
| 167 | 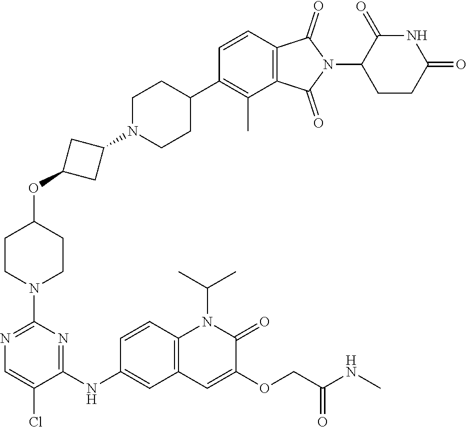 [SMILES: CNC(=O)COc1cc2cc(Nc3nc(N4CCC(O[C@H]5C[C@H](N6CCC(c7ccc8c(c7C)C(=O)N(C7CCC(=O)NC7=O)C8=O)CC6)C5)CC4)ncc3Cl)ccc2n(C(C)C)c1=O] | 2-({6-[(5-chloro-2-{4-[(1r,3r)-3-{4-[2- (2,6-dioxopiperidin-3-yl)-4-methyl-1,3- dioxo-2,3-dihydro-1H-isoindol-5- yl]piperidin-1-yl}cyclobutoxy]piperidin- 1-yl}pyrimidin-4-yl)amino]-2-oxo-1- (propan-2-yl)-1,2-dihydroquinolin-3- yl}oxy)-N-methylacetamide |
| 168 | 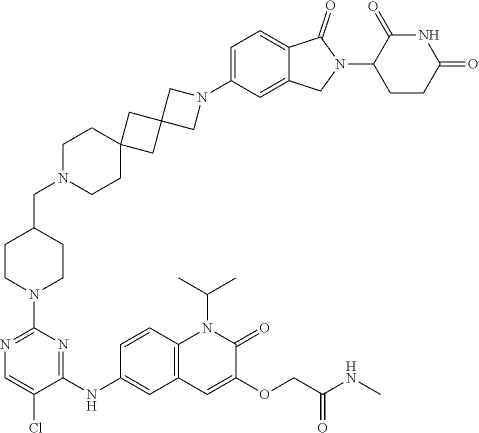 [SMILES: CNC(=O)COc1cc2cc(Nc3nc(N4CCC(CN5CCC6(CC5)CC5(CN(c7ccc8c(c7)CN(C7CCC(=O)NC7=O)C8=O)C5)C6)CC4)ncc3Cl)ccc2n(C(C)C)c1=O] | 2-{[6-({5-chloro-2-[4-({2-[2-(2,6- dioxopiperidin-3-yl)-1-oxo-2,3-dihydro- 1H-isoindol-5-yl]-2,9- diazadispio[3.1.5⁶.1⁴]dodecan-9- yl}methyl)piperidin-1-yl]pyrimidin-4- yl}amino)-2-oxo-1-(propan-2-yl)-1,2- dihydroquinolin-3-yl]oxy}-N- methylacetamide |
| 169 | 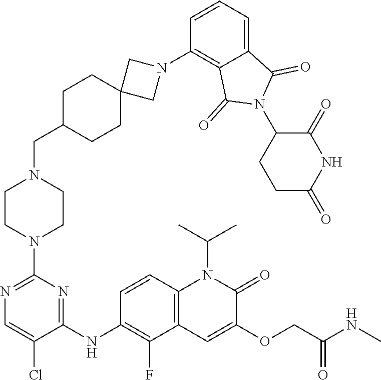 [SMILES: CNC(=O)COc1cc2c(F)c(Nc3nc(N4CCN(CC5CCC6(CC5)CN(c5cccc7c5C(=O)N(C5CCC(=O)NC5=O)C7=O)C6)CC4)ncc3Cl)ccc2n(C(C)C)c1=O] | 2-{[6-({5-chloro-2-[4-({2-[2-(2,6- dioxopiperidin-3-yl)-1,3-dioxo-2,3- dihydro-1H-isoindol-4-yl]-2- azaspiro[3.5]nonan-7- yl}methyl)piperazin-1-yl]pyrimidin-4- yl}amino)-5-fluoro-2-oxo-1-(propan-2- yl)-1,2-dihydroquinolin-3-yl]oxy}-N- methylacetamide |
| 170 | 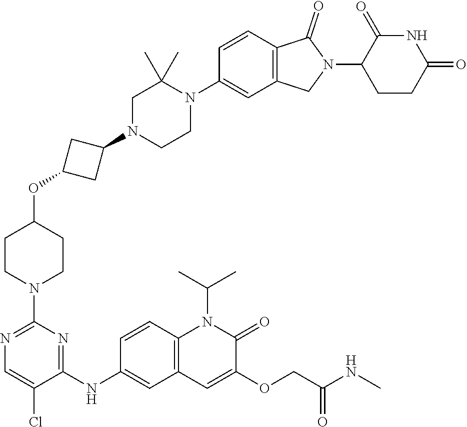 [SMILES: CNC(=O)COc1cc2cc(Nc3nc(N4CCC(O[C@H]5C[C@H](N6CCN(c7ccc8c(c7)CN(C7CCC(=O)NC7=O)C8=O)C(C)(C)C6)C5)CC4)ncc3Cl)ccc2n(C(C)C)c1=O] | 2-({6-[(5-chloro-2-{4-[(1r,3r)-3-{4-[2- (2,6-dioxopiperidin-3-yl)-1-oxo-2,3- dihydro-1H-isoindol-5-yl]-3,3- dimethylpiperazin-1- yl}cyclobutoxy]piperidin-1-yl}pyrimdiin- 4-yl)amino]-2-oxo-1-(propan-2-yl)-1,2- dihydroquinolin-3-yl}oxy)-N- methylacetamide |
| 171 | 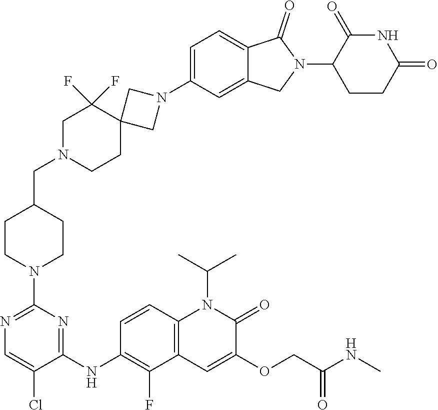 [SMILES: CNC(=O)COc1cc2c(F)c(Nc3nc(N4CCC(CN5CCC6(CN(c7ccc8c(c7)CN(C7CCC(=O)NC7=O)C8=O)C6)C(F)(F)C5)CC4)ncc3Cl)ccc2n(C(C)C)c1=O] | 2-{[6-({5-chloro-2-[4-({2-[2-(2,6- dioxopiperidin-3-yl)-1-oxo-2,3-dihydro- 1H-isoindol-5-yl]-5,5-difluoro-2,7- diazaspiro[3.5]nonan-7- yl}methyl)piperidin-1-yl]pyrimidin-4- yl}amino)-5-fluoro-2-oxo-1-(propan-2- yl)-1,2-dihydroquinolin-3-yl]oxy}-N- methylacetamide |
| 172 | 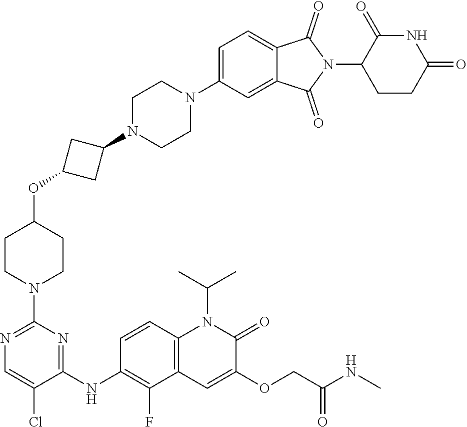 [SMILES: CNC(=O)COc1cc2c(F)c(Nc3nc(N4CCC(O[C@H]5C[C@H](N6CCN(c7ccc8c(c7)C(=O)N(C7CCC(=O)NC7=O)C8=O)CC6)C5)CC4)ncc3Cl)ccc2n(C(C)C)c1=O] | 2-({6-[(5-chloro-2-{4-[(1r,3r)-3-{4-[2- (2,6-dioxopiperidin-3-yl)-1,3-dioxo-2,3- dihydro-1H-isoindol-5-yl]piperidin-1- yl}cyclobutoxy]piperidin-1-yl}pyrimidin- 4-yl)amino]-5-fluoro-2-oxo-1-(propan-2- yl)-1,2-dihydroquinolin-3-yl}oxy)-N- methylacetamide |
| 173 | 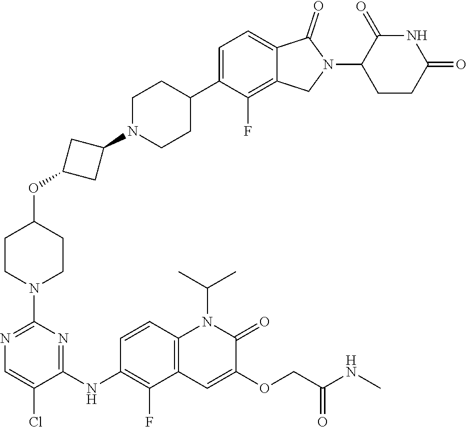 [SMILES: CNC(=O)COc1cc2c(F)c(Nc3nc(N4CCC(O[C@H]5C[C@H](N6CCC(c7ccc8c(c7F)CN(C7CCC(=O)NC7=O)C8=O)CC6)C5)CC4)ncc3Cl)ccc2n(C(C)C)c1=O] | 2-({6-[(5-chloro-2-{4-[(1r,3r)-3-{4-[2- (2,6-dioxopiperidin-3-yl)-4-fluoro-1-oxo- 2,3-dihydro-1H-isoindol-5-yl]piperidin-1- yl}cyclobutoxy]piperidin-1-yl}pyrimidin- 4-yl)amino]-5-fluoro-2-oxo-1-(propan-2- yl)-1,2-dihydroquinolin-3-yl}oxy)-N- methylacetamide |
| 174 | 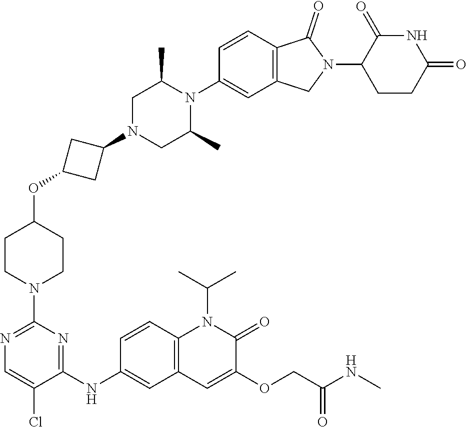 [SMILES: CNC(=O)COc1cc2cc(Nc3nc(N4CCC(O[C@H]5C[C@H](N6C[C@@H](C)N(c7ccc8c(c7)CN(C7CCC(=O)NC7=O)C8=O)[C@@H](C)C6)C5)CC4)ncc3Cl)ccc2n(C(C)C)c1=O] | 2-({6-[(5-chloro-2-{4-[(1r,3r)-3-[(3R,5S)- 4-[2-(2,6-dioxopiperidin-3-yl)-1-oxo-2,3- dihydro-1H-isoindol-5-yl]-3,5- dimethylpiperazin-1- yl]cyclobutoxy]piperidin-1-yl}pyrimidin- 4-yl)amino]-2-oxo-1-(propan-2-yl)-1,2- dihydroquinolin-3-yl}oxy)-N- methylacetamide |
| 175 | 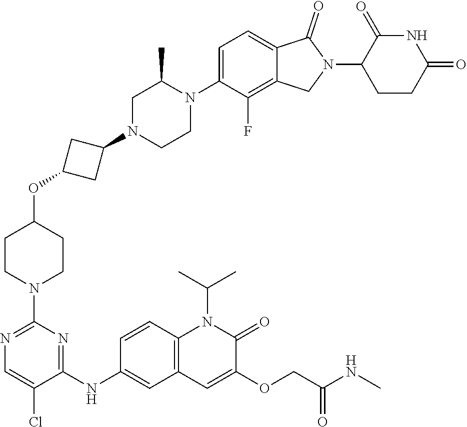 [SMILES: CNC(=O)COc1cc2cc(Nc3nc(N4CCC(O[C@H]5C[C@H](N6CCN(c7ccc8c(c7F)CN(C7CCC(=O)NC7=O)C8=O)[C@H](C)C6)C5)CC4)ncc3Cl)ccc2n(C(C)C)c1=O] | 2-({6-[(5-chloro-2-{4-[(1r,3r)-3-[(3R)-4- [2-(2,6-dioxopiperidin-3-yl)-4-fluoro-1- oxo-2,3-dihydro-1H-isoindol-5-yl]-3- methylpiperazin-1- yl]cyclobutoxy]piperidin-1-yl}pyrimidin- 4-yl)amino]-2-oxo-1-(propan-2-yl)-1,2- dihydroquinolin-3-yl}oxy)-N- methylacetamide |
| 176 | 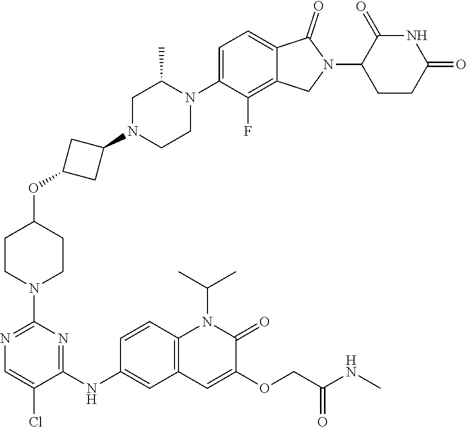 [SMILES: CNC(=O)COc1cc2cc(Nc3nc(N4CCC(O[C@H]5C[C@H](N6CCN(c7ccc8c(c7F)CN(C7CCC(=O)NC7=O)C8=O)[C@@H](C)C6)C5)CC4)ncc3Cl)ccc2n(C(C)C)c1=O] | 2-({6-[(5-chloro-2-{4-[(1r,3r)-3-[(3S)-4- [2-(2,6-dioxopiperidin-3-yl)-4-fluoro-1- oxo-2,3-dihydro-1H-isoindol-5-yl]-3- methylpiperazin-1- yl]cyclobutoxy]piperidin-1-yl}pyrimidin- 4-yl)amino]-2-oxo-1-(propan-2-yl)-1,2- dihydroquinolin-3-yl}oxy)-N- methylacetamide |
| 177 | 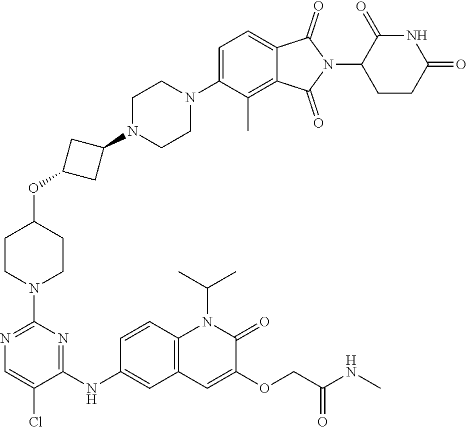 [SMILES: CNC(=O)COc1cc2cc(Nc3nc(N4CCC(O[C@H]5C[C@H](N6CCN(c7ccc8c(c7C)C(=O)N(C7CCC(=O)NC7=O)C8=O)CC6)C5)CC4)ncc3Cl)ccc2n(C(C)C)c1=O] | 2-({6-[(5-chloro-2-{4-[(1r,3r)-3-{4-[2- (2,6-dioxopiperidin-3-yl)-4-methyl-1,3- dioxo-2,3-dihydro-1H-isoindol-5- yl]piperazin-1-yl}cyclobutoxy]piperidin- 1-yl}pyrimidin-4-yl)amino]-2-oxo-1- (propan-2-yl)-1,2-dihydroquinolin-3- yl}oxy)-N-methylacetamide |
| 178 | 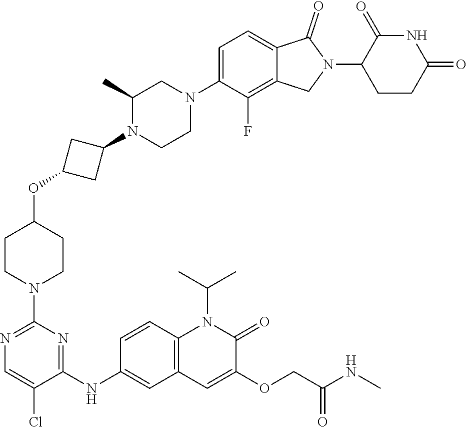 [SMILES: CNC(=O)COc1cc2cc(Nc3nc(N4CCC(O[C@H]5C[C@H](N6CCN(c7ccc8c(c7F)CN(C7CCC(=O)NC7=O)C8=O)C[C@@H]6C)C5)CC4)ncc3Cl)ccc2n(C(C)C)c1=O] | 2-({6-[(5-chloro-2-{4-[(1r,3r)-3-[(2S)-4- [2-(2,6-dioxopiperidin-3-yl)-4-fluoro-1- oxo-2,3-dihydro-1H-isoindol-5-yl]-2- methylpiperazin-1- yl]cyclobutoxy]piperidin-1-yl}pyrimidin- 4-yl)amino]-2-oxo-1-(propan-2-yl)-1,2- dihydroquinolin-3-yl}oxy)-N- methylacetamide |
| 179 | 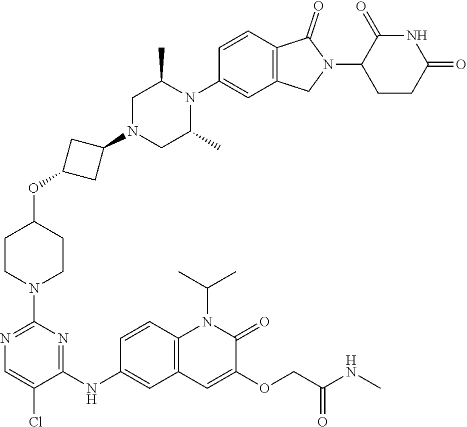 [SMILES: CNC(=O)COc1cc2cc(Nc3nc(N4CCC(O[C@H]5C[C@H](N6C[C@@H](C)N(c7ccc8c(c7)CN(C7CCC(=O)NC7=O)C8=O)[C@H](C)C6)C5)CC4)ncc3Cl)ccc2n(C(C)C)c1=O] | 2-({6-[(5-chloro-2-{4-[(1r,3r)-3- [(3R*,5R*)-4-[2-(2,6-dioxopiperidin-3- yl)-1-oxo-2,3-dihydro-1H-isoindol-5-yl]- 3,5-dimethylpiperazin-1- yl]cyclobutoxy]piperidin-1-yl}pyrimidin- 4-yl)amino]-2-oxo-1-(propan-2-yl)-1,2- dihydroquinolin-3-yl}oxy)-N- methylacetamide |
| 180 | 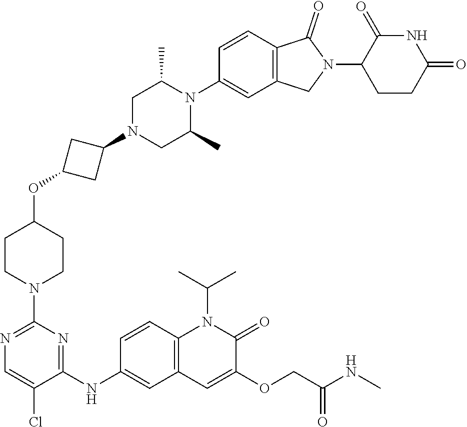 [SMILES: CNC(=O)COc1cc2cc(Nc3nc(N4CCC(O[C@H]5C[C@H](N6C[C@H](C)N(c7ccc8c(c7)CN(C7CCC(=O)NC7=O)C8=O)[C@@H](C)C6)C5)CC4)ncc3Cl)ccc2n(C(C)C)c1=O] | 2-({6-[(5-chloro-2-{4-[(1r,3r)-3- [(3R*,5R*)-4-[2-(2,6-dioxopiperidin-3- yl)-1-oxo-2,3-dihydro-1H-isoindol-5-yl]- 3,5-dimethylpiperazin-1- yl]cyclobutoxy]piperidin-1-yl}pyrimidin- 4-yl)amino]-2-oxo-1-(propan-2-yl)-1,2- dihydroquinolin-3-yl}oxy)-N- methylacetamide |
| 181 | 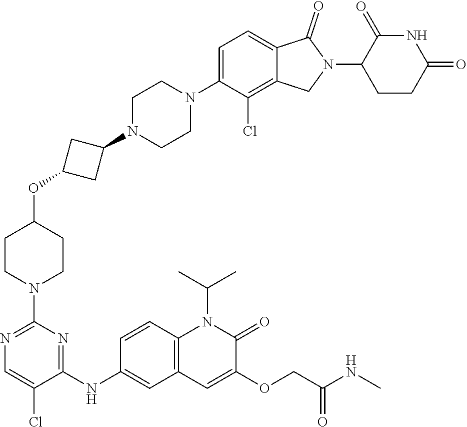 [SMILES: CNC(=O)COc1cc2cc(Nc3nc(N4CCC(O[C@H]5C[C@H](N6CCN(c7ccc8c(c7Cl)CN(C7CCC(=O)NC7=O)C8=O)CC6)C5)CC4)ncc3Cl)ccc2n(C(C)C)c1=O] | 2-({6-[(5-chloro-2-{4-[(1r,3r)-3-{4-[4- chloro-2-(2,6-dioxopiperidin-3-yl)-1-oxo- 2,3-dihydro-1H-isoindol-5-l]piperazin-1- yl}cyclobutoxy]piperidin-1-yl}pyrimidin- 4-yl)amino]-2-oxo-1-(propan-2-yl)-1,2- dihydroquinolin-3-yl}oxy)-N- methylacetamide |
| 182 | 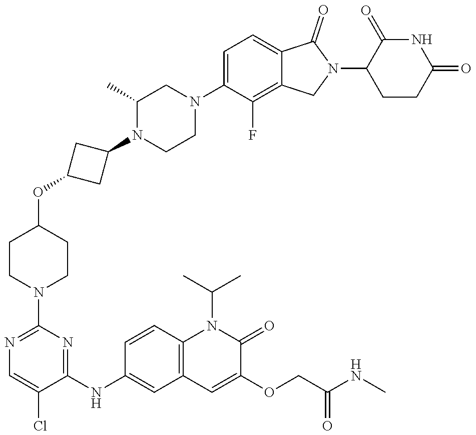 [SMILES: CNC(=O)COc1cc2cc(Nc3nc(N4CCC(O[C@H]5C[C@H](N6CCN(c7ccc8c(c7F)CN(C7CCC(=O)NC7=O)C8=O)C[C@H]6C)C5)CC4)ncc3Cl)ccc2n(C(C)C)c1=O] | 2-({6-[(5-chloro-2-{4-[(1r,3r)-3-[(2R)-4- [2-(2,6-dioxopiperidin-3-yl)-4-fluoro-1- oxo-2,3-dihydro-1H-isoindol-5-yl]-2- methylpiperazin-1- yl]cyclobutoxy]piperidin-1-yl}pyrimidin- 4-yl)amino]-2-oxo-1-(propan-2-yl)-1,2- dihydroquinolin-3-yl}oxy)-N- methylacetamide |
| 183 | 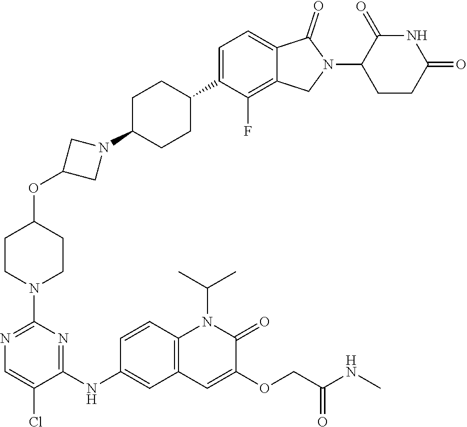 [SMILES: CNC(=O)COc1cc2cc(Nc3nc(N4CCC(OC5CN([C@H]6CC[C@H](c7ccc8c(c7F)CN(C7CCC(=O)NC7=O)C8=O)CC6)C5)CC4)ncc3Cl)ccc2n(C(C)C)c1=O] | 2-{[6-({5-chloro-2-[4-({1-[(1r,4r)-4-[2- (2,6-dioxopiperidin-3-yl)-4-fluoro-1-oxo- 2,3-dihydro-1H-isoindol-5- yl]cyclohexyl]azetidin-3- yl}oxy)piperidin-1-yl]pyrimidin-4- yl}amino)-2-oxo-1-(propan-2-yl)-1,2- dihydroquinolin-3-yl]oxy}-N- methylacetamide |
| 184 | 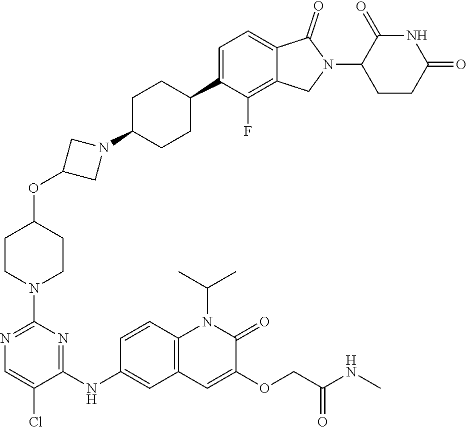 [SMILES: CNC(=O)COc1cc2cc(Nc3nc(N4CCC(OC5CN([C@H]6CC[C@@H](c7ccc8c(c7F)CN(C7CCC(=O)NC7=O)C8=O)CC6)C5)CC4)ncc3Cl)ccc2n(C(C)C)c1=O] | 2-{[6-({5-chloro-2-[4-({1-[(1s,4s)-4-[2- (2,6-dioxopiperidin-3-yl)-4-fluoro-1-oxo- 2,3-dihydro-1H-isoindol-5- yl]cyclohexyl]azetidin-3- yl}oxy)piperidin-1-yl]pyrimidin-4- yl}amino)-2-oxo-1-(propan-2-yl)-1,2- dihydroquinolin-3-yl]oxy}-N- methylacetamide |
| 185 | 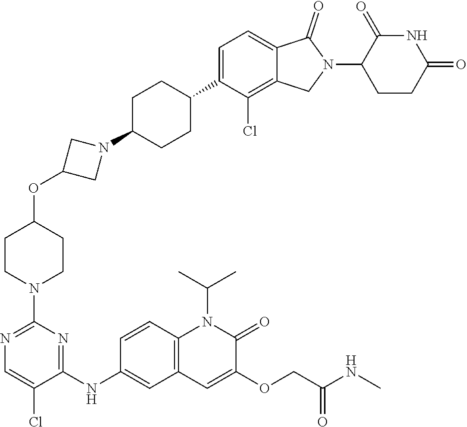 [SMILES: CNC(=O)COc1cc2cc(Nc3nc(N4CCC(OC5CN([C@H]6CC[C@H](c7ccc8c(c7Cl)CN(C7CCC(=O)NC7=O)C8=O)CC6)C5)CC4)ncc3Cl)ccc2n(C(C)C)c1=O] | 2-{[6-({5-chloro-2-[4-({1-[(1r,4r)-4-[4- chloro-2-(2,6-dioxopiperidin-3-yl)-1-oxo- 2,3-dihydro-1H-isoindol-5- yl]cyclohexyl]azetidin-3- yl}oxy)piperidin-1-yl]pyrimidin-4- yl}amino)-2-oxo-1-(propan-2-yl)-1,2- dihydroquinolin-3-yl]oxy}-N- methylacetamide |
| 186 | 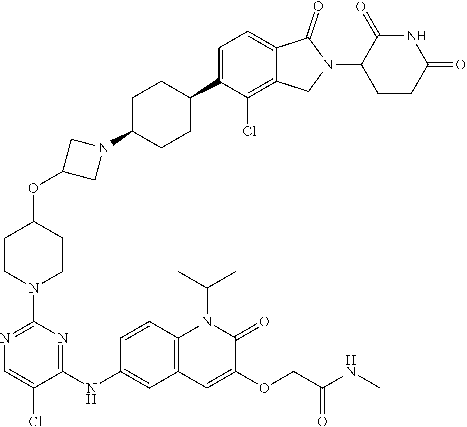 [SMILES: CNC(=O)COc1cc2cc(Nc3nc(N4CCC(OC5CN([C@H]6CC[C@@H](c7ccc8c(c7Cl)CN(C7CCC(=O)NC7=O)C8=O)CC6)C5)CC4)ncc3Cl)ccc2n(C(C)C)c1=O] | 2-{[6-({5-chloro-2-[4-({1-[(1s,4s)-4-[4- chloro-2-(2,6-dioxopiperidin-3-yl)-1-oxo- 2,3-dihydro-1H-isoindol-5- yl]cyclohexyl]azetidin-3- yl}oxy)piperidin-1-yl]pyrimidin-4- yl}amino)-2-oxo-1-(propan-2-yl)-1,2- dihydroquinolin-3-yl]oxy}-N- methylacetamide |
| 187 | 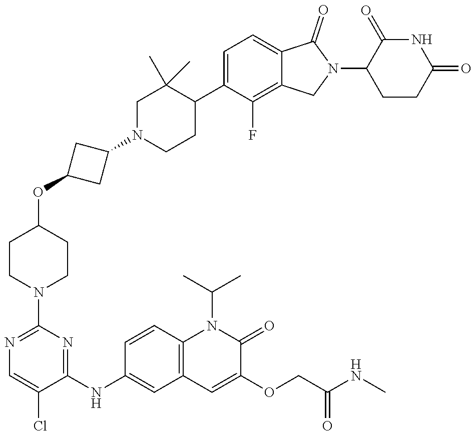 [SMILES: CNC(=O)COc1cc2cc(Nc3nc(N4CCC(O[C@H]5C[C@H](N6CCC(c7ccc8c(c7F)CN(C7CCC(=O)NC7=O)C8=O)C(C)(C)C6)C5)CC4)ncc3Cl)ccc2n(C(C)C)c1=O] | 2-({6-[(5-chloro-2-{4-[(1r,3r)-3-{4-[2- (2,6-dioxopiperidin-3-yl)-4-fluoro-1-oxo- 2,3-dihydro-1H-isoindol-5-yl]-3,3- dimethylpiperidin-1- yl}cyclobutoxy]piperidin-1-yl}pyrimidin- 4-yl)amino]-2-oxo-1-(propan-2-l)-1,2- dihydroquinolin-3-yl}oxy)-N- methylacetamide |
| 188 | 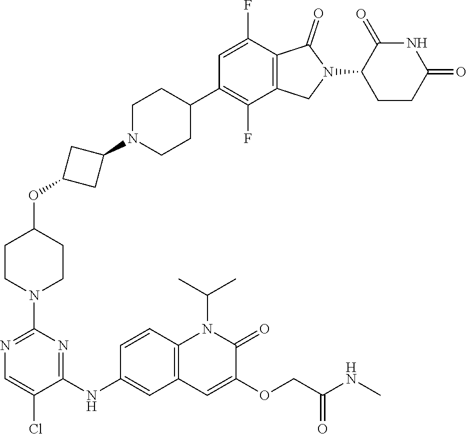 [SMILES: CNC(=O)COc1cc2cc(Nc3nc(N4CCC(O[C@H]5C[C@H](N6CCC(c7cc(F)c8c(c7F)CN([C@H]7CCC(=O)NC7=O)C8=O)CC6)C5)CC4)ncc3Cl)ccc2n(C(C)C)c1=O] | 2-({6-[(5-chloro-2-{4-[(1r,3r)-3-(4-{2- [(3S)-2,6-dioxopiperidin-3-yl]-4,7- difluoro-1-oxo-2,3-dihydro-1H-isoindol- 5-yl}piperidin-1- yl)cyclobutoxy]piperidin-1-yl}pyrimidin- 4-yl)amino]-2-oxo-1-(propan-2-yl)-1,2- dihydroquinolin-3-yl}oxy)-N- methylacetamide |
| 189 | 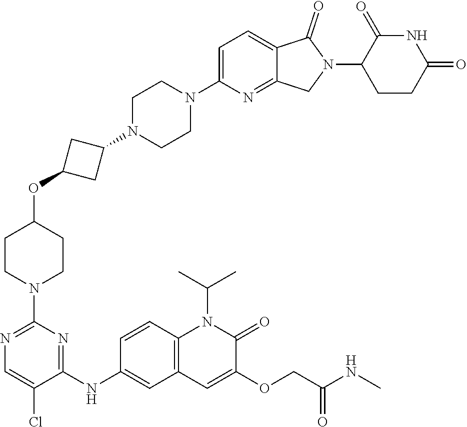 [SMILES: CNC(=O)COc1cc2cc(Nc3nc(N4CCC(O[C@H]5C[C@H](N6CCN(c7ccc8c(n7)CN(C7CCC(=O)NC7=O)C8=O)CC6)C5)CC4)ncc3Cl)ccc2n(C(C)C)c1=O] | 2-({6-[(5-chloro-2-{4-[(1r,3r)-3-{4-[6- (2,6-dioxopiperidin-3-yl)-5-oxo- 5H,6H,7H-pyrrolo[3,4-b]pyridin-2- yl]piperazin-1-yl}cyclobutoxy]piperidin- 1-yl}pyrimidin-4-yl)amino]-2-oxo-1- (propan-2-yl)-1,2-dihydroquinolin-3- yl}oxy)-N-methylacetamide |
| 190 | 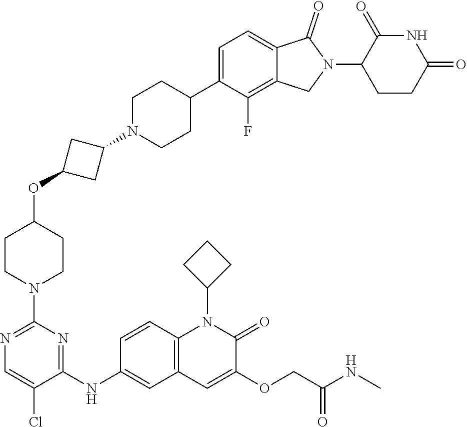 [SMILES: CNC(=O)COc1cc2cc(Nc3nc(N4CCC(O[C@H]5C[C@H](N6CCC(c7ccc8c(c7F)CN(C7CCC(=O)NC7=O)C8=O)CC6)C5)CC4)ncc3Cl)ccc2n(C2CCC2)c1=O] | 2-({6-[(5-chloro-2-{4-[(1r,3r)-3-{4-[2- (2,6-dioxopiperidin-3-yl)-4-fluoro-1-oxo- 2,3-dihydro-1H-isoindol-5-yl]piperidin-1- yl}cyclobutoxy]piperidin-1-yl}pyrimidin- 4-yl)amino]-1-cyclobutyl-2-oxo-1,2- dihydroquinolin-3-yl}oxy)-N- methylacetamide |
| 191 | 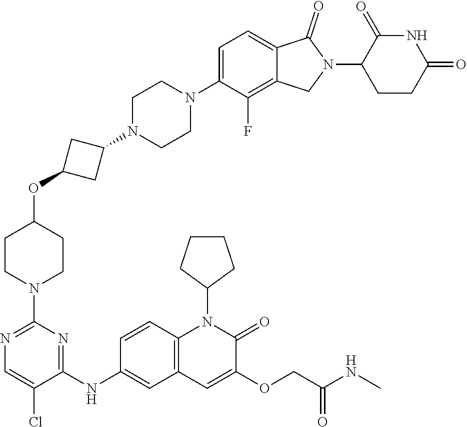 [SMILES: CNC(=O)COc1cc2cc(Nc3nc(N4CCC(O[C@H]5C[C@H](N6CCN(c7ccc8c(c7F)CN(C7CCC(=O)NC7=O)C8=O)CC6)C5)CC4)ncc3Cl)ccc2n(C2CCCC2)c1=O] | 2-({6-[(5-chloro-2-{4-[(1r,3r)-3-{4-[2- (2,6-dioxopiperidin-3-yl)-4-fluoro-1-oxo- 2,3-dihydro-1H-isoindol-5-yl]piperidin-1- yl}cyclobutoxy]piepridin-1-yl}pyrimidin- 4-yl)amino]-1-cyclopentyl-2-oxo-1,2- dihydroquinolin-3-yl}oxy)-N- methylacetamide |
| 192 | 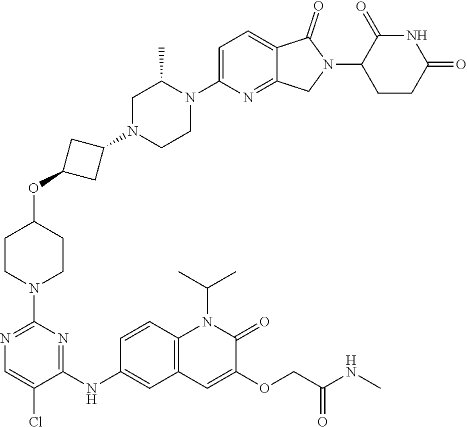 [SMILES: CNC(=O)COc1cc2cc(Nc3nc(N4CCC(O[C@H]5C[C@H](N6CCN(c7ccc8c(n7)CN(C7CCC(=O)NC7=O)C8=O)[C@@H](C)C6)C5)CC4)ncc3Cl)ccc2n(C(C)C)c1=O] | 2-({6-[(5-chloro-2-{4-[(1r,3r)-3-[(3S)-4- [6-(2,6-dioxopiperidin-3-yl)-5-oxo- 5H,6H,7H-pyrrolo[3,4-b]pyridin-2-yl]-3- methylpiperazin-1- yl]cyclobutoxy]piperidin-1-yl}pyrimidin- 4-yl)amino]-2-oxo-1-(propan-2-yl)-1,2- dihydroquinolin-3-yl}oxy)-N- methylacetamide |
| 193 | 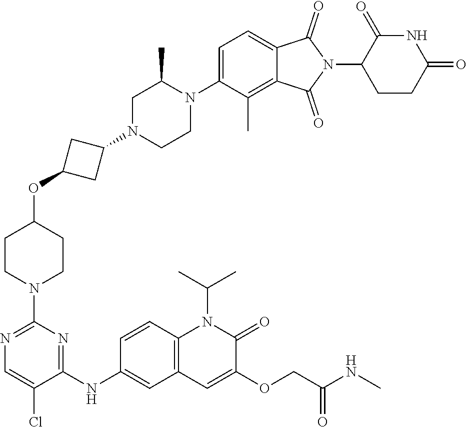 [SMILES: CNC(=O)COc1cc2cc(Nc3nc(N4CCC(O[C@H]5C[C@H](N6CCN(c7ccc8c(c7C)C(=O)N(C7CCC(=O)NC7=O)C8=O)[C@H](C)C6)C5)CC4)ncc3Cl)ccc2n(C(C)C)c1=O] | 2-({6-[(5-chloro-2-{4-[(1r,3r)-3-[(3R)-4- [2-(2,6-dioxopiperidin-3-yl)-4-methyl- 1,3-dioxo-2,3-dihydro-1H-isoindol-5-yl]- 3-methylpiperazin-1- yl]cyclobutoxy]piperidin-1-yl}pyrimidin- 4-yl)amino]-2-oxo-1-(propan-2-yl)-1,2- dihydroquinolin-3-yl}oxy)-N- methylacetamide |
| 194 | 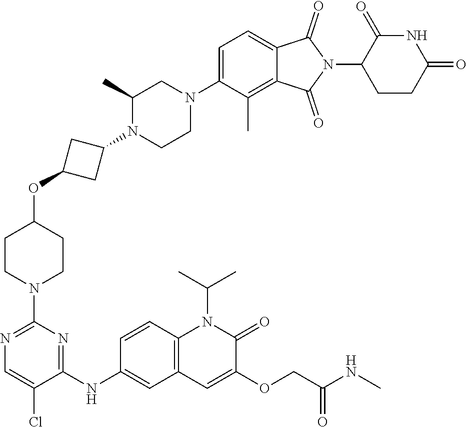 [SMILES: CNC(=O)COc1cc2cc(Nc3nc(N4CCC(O[C@H]5C[C@H](N6CCN(c7ccc8c(c7C)C(=O)N(C7CCC(=O)NC7=O)C8=O)C[C@@H]6C)C5)CC4)ncc3Cl)ccc2n(C(C)C)c1=O] | 2-({6-[(5-chloro-2-{4-[(1r,3r)-3-[(2S)-4- [2-(2,6-dioxopiperidin-3-yl)-4-methyl- 1,3-dioxo-2,3-dihydro-1H-isoindol-5-yl]- 2-methylpiperazin-1- yl]cyclobutoxy]piperidin-1-yl}pyrimdiin- 4-yl)amino]-2-oxo-1-(propan-2-yl)-1,2- dihydroquinolin-3-yl}oxy)-N- methylacetamide |
| 195 | 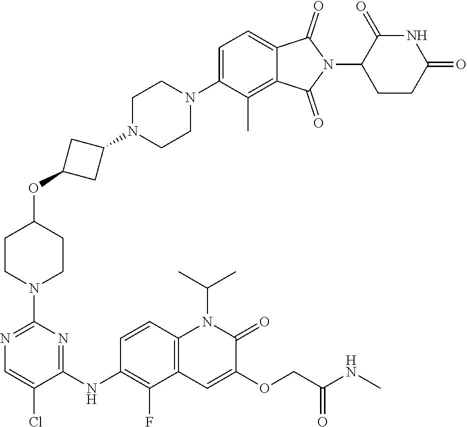 [SMILES: CNC(=O)COc1cc2c(F)c(Nc3nc(N4CCC(O[C@H]5C[C@H](N6CCN(c7ccc8c(c7C)C(=O)N(C7CCC(=O)NC7=O)C8=O)CC6)C5)CC4)ncc3Cl)ccc2n(C(C)C)c1=O] | 2-({6-[(5-chloro-2-{4-[(1r,3r)-3-{4-[2- (2,6-dioxopiperidin-3-yl)-4-methyl-1,3- dioxo-2,3-dihydro-1H-isoindol-5- yl]piperazin-1-yl}cyclobutoxy]piperidn- 1-yl}pyrimidin-4-yl)amino]-5-fluoro-2- oxo-1-(propan-2-yl)-1,2-dihydroquinolin- 3-yl}oxy)-N-methylacetamide |
| 196 | 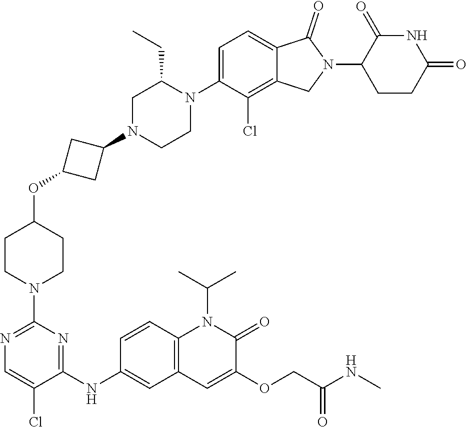 [SMILES: CC[C@H]1CN([C@H]2C[C@H](OC3CCN(c4ncc(Cl)c(Nc5ccc6c(c5)cc(OCC(=O)NC)c(=O)n6C(C)C)n4)CC3)C2)CCN1c1ccc2c(c1Cl)CN(C1CCC(=O)NC1=O)C2=O] | 2-({6-[(5-chloro-2-{4-[(1r,3r)-3-[(3S)-4- [4-chloro-2-(2,6-dioxopiperidin-3-yl)-1- oxo-2,3-dihydro-1H-isoindol-5-yl]-3- ethylpiperazin-1- yl]cyclobutoxy]piperidin-1-yl}pyrimidin- 4-yl)amino]-2-oxo-1-(proapn-2-yl)-1,2- dihydroquinolin-3-yl}oxy)-N- methylacetamide |
| 197 | 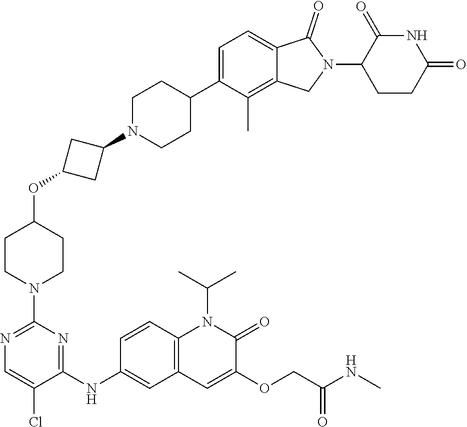 [SMILES: CNC(=O)COc1cc2cc(Nc3nc(N4CCC(O[C@H]5C[C@H](N6CCC(c7ccc8c(c7C)CN(C7CCC(=O)NC7=O)C8=O)CC6)C5)CC4)ncc3Cl)ccc2n(C(C)C)c1=O] | 2-({6-[(5-chloro-2-{4-[(1r,3r)-3-{4-[2- (2,6-dioxopiperidin-3-yl)-4-methyl-1-oxo- 2,3-dihydro-1H-isoindol-5-yl]piperidin-1- yl}cyclobutoxy]piperidin-1-yl}pyrimidin- 4-yl)amino]-2-oxo-1-(propan-2-yl)-1,2- dihydroquinolin-3-yl}oxy)-N- methylacetamide |
| 198 | 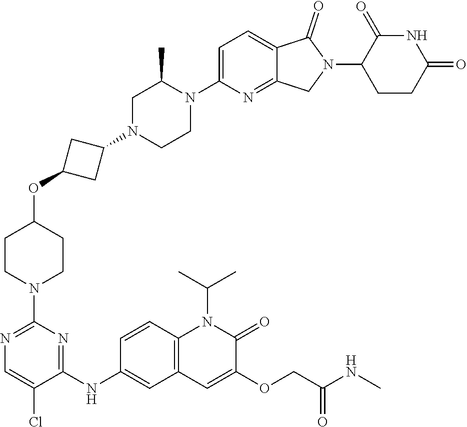 [SMILES: CNC(=O)COc1cc2cc(Nc3nc(N4CCC(O[C@H]5C[C@H](N6CCN(c7ccc8c(n7)CN(C7CCC(=O)NC7=O)C8=O)[C@H](C)C6)C5)CC4)ncc3Cl)ccc2n(C(C)C)c1=O] | 2-({6-[(5-chlroo-2-{4-[(1r,3r)-3-[(3R)-4- [6-(2,6-dioxopiperidin-3-yl)-5-oxo- 5H,6H,7H-pyrrolo[3,4-b]pyridin-2-yl]-3- methylpiperazin-1- yl]cyclobutoxy]piperidin-1-yl}pyrimidin- 4-yl)amino]-2-oxo-1-(propan-2-yl)-1,2- dihydroquinolin-3-yl}oxy)-N- methylacetamide |
| 199 | 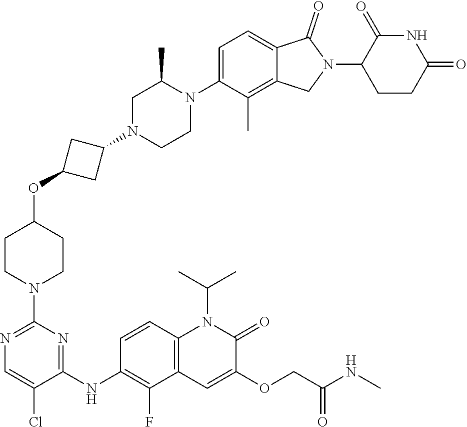 [SMILES: CNC(=O)COc1cc2c(F)c(Nc3nc(N4CCC(O[C@H]5C[C@H](N6CCN(c7ccc8c(c7C)CN(C7CCC(=O)NC7=O)C8=O)[C@H](C)C6)C5)CC4)ncc3Cl)ccc2n(C(C)C)c1=O] | 2-({6-[(5-chloro-2-{4-[(1r,3R)-3-[(3R)-4- [2-(2,6-dioxopiperidin-3-yl)-4-methyl- 1,3-dioxo-2,3-dihydro-1H-isoindol-5-yl]- 3-methylpiperazin-1- yl]cyclobutoxy]piperidin-1-yl}pyrimidin- 4-yl)amino]-5-fluoro-2-oxo-1-(propan-2- yl)-1,2-dihydroquinolin-3-yl}oxy)-N- methylacetamide |
| 200 | 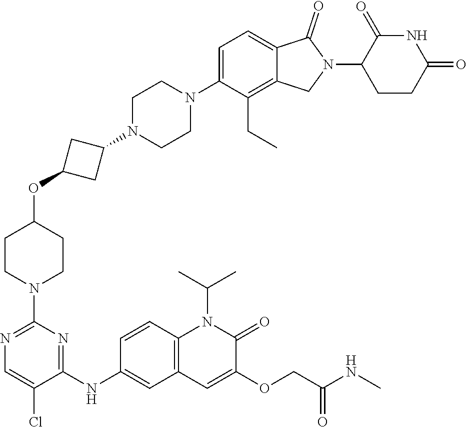 [SMILES: CCc1c(N2CCN([C@H]3C[C@H](OC4CCN(c5ncc(Cl)c(Nc6ccc7c(c6)cc(OCC(=O)NC)c(=O)n7C(C)C)n5)CC4)C3)CC2)ccc2c1CN(C1CCC(=O)NC1=O)C2=O] | 2-({6-[(5-chloro-2-{4-[(1r,3r)-3-{4-[2- (2,6-dioxopiperidin-3-yl)-4-ethyl-1,3- dioxo-2,3-dihydro-1H-isoindol-5- yl]piperazin-1-yl}cyclobutoxy]piperidin- 1-yl}pyrimidin-4-yl)amino]-2-oxo-1- (propan-2-yl)-1,2-dihydroquinolin-3- yl}oxy)-N-methylacetamide |
| 201 | 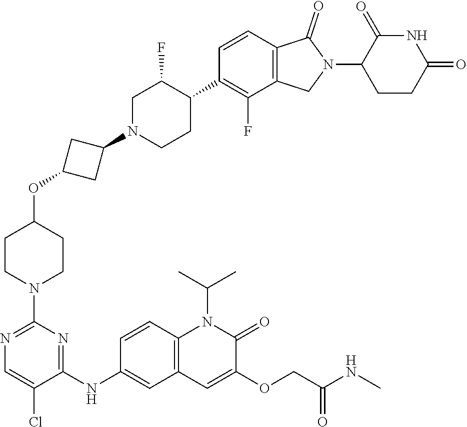 [SMILES: CNC(=O)COc1cc2cc(Nc3nc(N4CCC(O[C@H]5C[C@H](N6CC[C@@H](c7ccc8c(c7F)CN(C7CCC(=O)NC7=O)C8=O)[C@@H](F)C6)C5)CC4)ncc3Cl)ccc2n(C(C)C)c1=O] | 2-({6-[(5-chloro-2-{4-[(1r,3r)-3-[(3R,4S)- 4-[2-(2,6-dioxopiperidin-3-yl)-4-fluoro-1- oxo-2,3-dihydro-1H-isoindol-5-yl]-3- fluoropiperidin-1- yl]cyclobutoxy]piperidin-1-yl}pyrimidin- 4-yl)amino]-2-oxo-1-(propan-2-yl)-1,2- dihydroquinolin-3-yl}oxy)-N- methylacetamide |
| 202 | 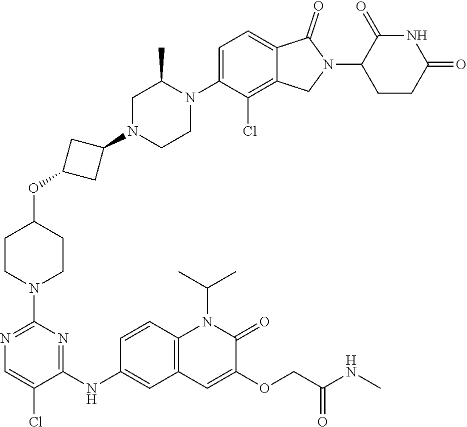 [SMILES: CNC(=O)COc1cc2cc(Nc3nc(N4CCC(O[C@H]5C[C@H](N6CCN(c7ccc8c(c7Cl)CN(C7CCC(=O)NC7=O)C8=O)[C@H](C)C6)C5)CC4)ncc3Cl)ccc2n(C(C)C)c1=O] | 2-({6-[(5-chloro-2-{4-[(1r,3r)-3-[(3R)-4- [4-chloro-2-(2,6-dioxopiperidin-3-yl)-1- oxo-2,3-dihydro-1H-isoindol-5-yl]-3- methylpiperazin-1- yl]cyclobutoxy]piperidin-1-yl}pyrimidin- 4-yl)amino]-2-oxo-1-(propan-2-yl)-1,2- dihydroquinolin-3-yl}oxy)-N- methylacetamide |
| 203 | 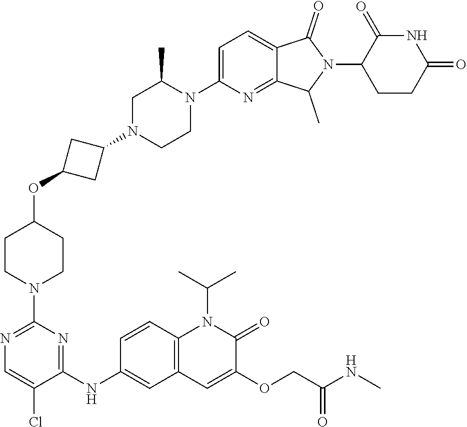 [SMILES: CNC(=O)COc1cc2cc(Nc3nc(N4CCC(O[C@H]5C[C@H](N6CCN(c7ccc8c(n7)C(C)N(C7CCC(=O)NC7=O)C8=O)[C@H](C)C6)C5)CC4)ncc3Cl)ccc2n(C(C)C)c1=O] | 2-({6-[(5-chloro-2-{4-[(1r,3r)-3-[(3R)-4- [6-(2,6-dioxopiperidin-3-yl)-7-methyl-5- oxo-5H,6H,7H-pyrrolo[3,4-b]pyridin-2- yl]-3-methylpiperazin-1- yl]cyclobutoxy]piepridin-1-yl}pyrimidin- 4-yl)amino]-2-oxo-1-(propan-2-yl)-1,2- dihydroquinolin-3-yl}oxy)-N- methylacetamide |
| 204 | 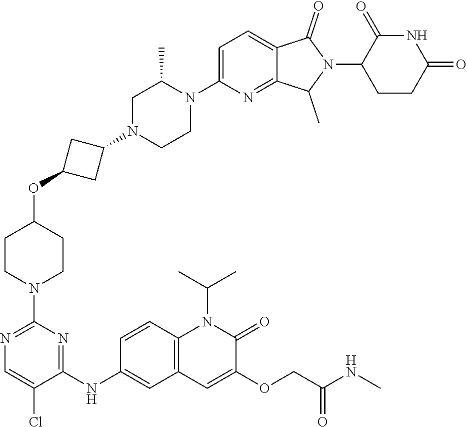 [SMILES: CNC(=O)COc1cc2cc(Nc3nc(N4CCC(O[C@H]5C[C@H](N6CCN(c7ccc8c(n7)C(C)N(C7CCC(=O)NC7=O)C8=O)[C@@H](C)C6)C5)CC4)ncc3Cl)ccc2n(C(C)C)c1=O] | 2-({6-[(5-chloro-2-{4-[(1r,3r)-3-[(3S)-4- [6-(2,6-dioxopiperidin-3-yl)-7-methyl-5- oxo-5H,6H,7H)-pyrrolo[3,4-b]pyridin-2- yl]-3-methylpiperazin-1- yl]cyclobutoxy]piperidin-1-yl}pyrimidin- 4-yl)amino]-2-oxo-1-(propan-2-yl)-1,2- dihydroquinolin-3-yl}oxy)-N- methylacetamide |
| 205 | 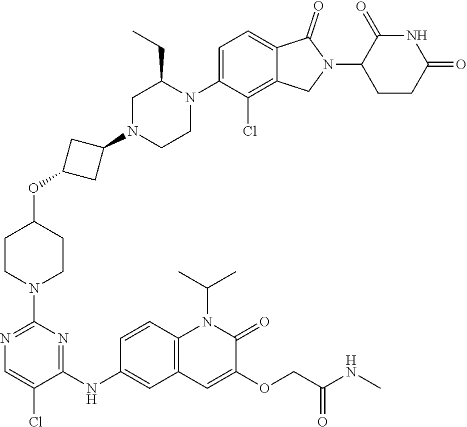 [SMILES: CC[C@@H]1CN([C@H]2C[C@H](OC3CCN(c4ncc(Cl)c(Nc5ccc6c(c5)cc(OCC(=O)NC)c(=O)n6C(C)C)n4)CC3)C2)CCN1c1ccc2c(c1Cl)CN(C1CCC(=O)NC1=O)C2=O] | 2-({6-[(5-chloro-2-{4-[(1r,3r)-3-[(3R)-4- [4-chloro-2-(2,6-dioxopiperidin-3-yl)-1- oxo-2,3-dihydro-1H-isoindol-5-yl]-3- ethylpiperazin-1- yl]cyclobutoxy]piperidin-1-yl}pyrimidin- 4-yl)amino]-2-oxo-1-(propan-2-yl)-1,2- dihydroquinolin-3-yl}oxy)-N- methylacetamide |
| 206 | 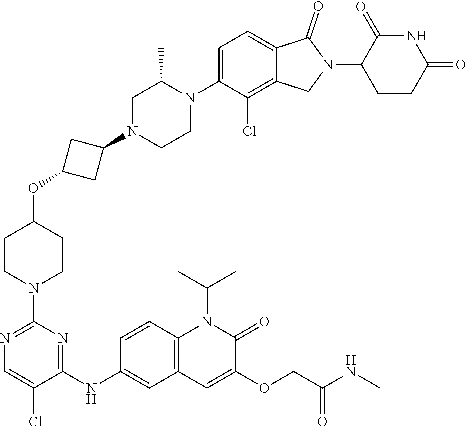 [SMILES: CNC(=O)COc1cc2cc(Nc3nc(N4CCC(O[C@H]5C[C@H](N6CCN(c7ccc8c(c7Cl)CN(C7CCC(=O)NC7=O)C8=O)[C@@H](C)C6)C5)CC4)ncc3Cl)ccc2n(C(C)C)c1=O] | 2-({6-[(5-chloro-2-{4-[(1r,3r)-3-[(3S)-4- [4-chloro-2-(2,6-dioxopiperidin-3-yl)-1- oxo-2,3-dihydro-1H-isoindol-5-yl]-3- methylpiperazin-1- yl]cyclobutoxy]piperidin-1-yl}pyrimidin- 4-yl)amino]-2-oxo-1-(propan-2-yl)-1,2- dihydroquinolin-3-yl}oxy)-N- methylacetamide |
| 207 | 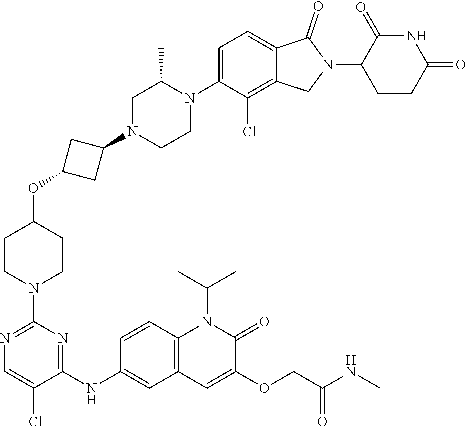 [SMILES: CNC(=O)COc1cc2cc(Nc3nc(N4CCC(O[C@H]5C[C@H](N6CCN(c7ccc8c(c7Cl)CN(C7CCC(=O)NC7=O)C8=O)[C@@H](C)C6)C5)CC4)ncc3Cl)ccc2n(C(C)C)c1=O] | 2-({6-[(5-chloro-2-{4-[(1r,3r)-3-[(2S,5R)- 4-[2-(2,6-dioxopiperidin-3-yl)-4-methyl- 1,3-dioxo-2,3-dihydro-1H-isoindol-5-yl]- 2,5-dimethylpiperazin-1- yl]cyclobutoxy]piperidin-1-yl}pyrimidin- 4-yl)amino]-2-oxo-1-(propan-2-yl)-1,2- dihydroquinolin-3-yl}oxy)-N- methylacetamide |
| 208 | 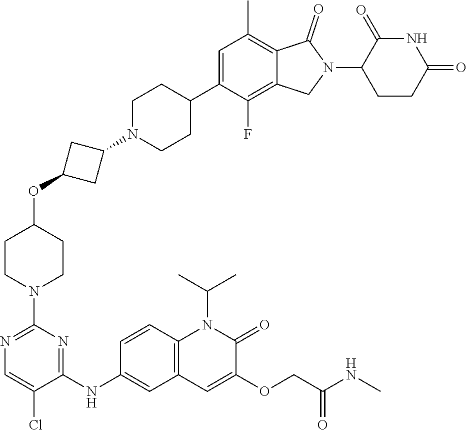 [SMILES: CNC(=O)COc1cc2cc(Nc3nc(N4CCC(O[C@H]5C[C@H](N6CCC(c7cc(C)c8c(c7F)CN(C7CCC(=O)NC7=O)C8=O)CC6)C5)CC4)ncc3Cl)ccc2n(C(C)C)c1=O] | 2-({6-[(5-chloro-2-{4-[(1r,3r)-3-{4-[2- (2,6-dioxopiperidin-3-yl)-4-fluoro-7- methyl-1-oxo-2,3-dihydro-1H-isoindol-5- yl]piperidin-1-yl}cyclobutoxy]piperidin- 1-yl}pyrimidin-4-yl)amino]-2-oxo-1- (propan-2-yl)-1,2-dihydroquinolin-3- yl}oxy)-N-methylacetamide |
| 209 | 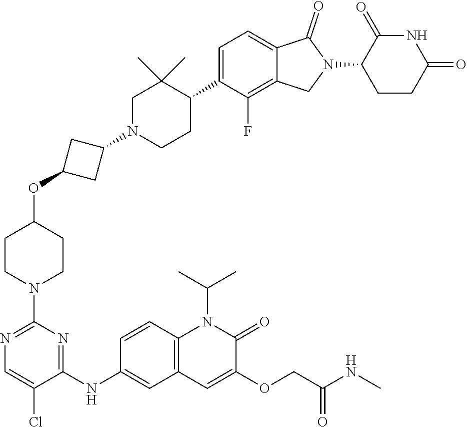 [SMILES: CNC(=O)COc1cc2cc(Nc3nc(N4CCC(O[C@H]5C[C@H](N6CC[C@@H](c7ccc8c(c7F)CN([C@H]7CCC(=O)NC7=O)C8=O)C(C)(C)C6)C5)CC4)ncc3Cl)ccc2n(C(C)C)c1=O] | 2-({6-[(5-chloro-2-{4-[(1r,3r)-3-[(4R)-4- {2-[(3S)-2,6-dioxopiperidin-3-yl]-4- fluoro-1-oxo-2,3-dihydro-1H-isoindol-5- yl}-3,3-dimethylpiperidin-1- yl]cyclobutoxy]piperiidn-1-yl}pyrimidin- 4-yl)amino]-2-oxo-1-(propan-2-yl)-1,2- dihydroquinolin-3-yl}oxy)-N- methylacetamide |
| 210 | 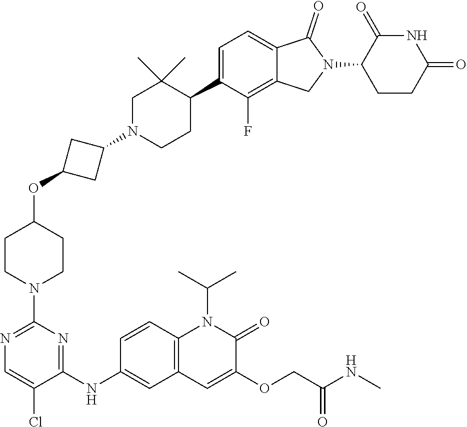 [SMILES: CNC(=O)COc1cc2cc(Nc3nc(N4CCC(O[C@H]5C[C@H](N6CC[C@H](c7ccc8c(c7F)CN([C@H]7CCC(=O)NC7=O)C8=O)C(C)(C)C6)C5)CC4)ncc3Cl)ccc2n(C(C)C)c1=O] | 2-({6-[(5-chloro-2-{4-[(1r,3r)-3-[(4S)-4- {2-[(3S)-2,6-dioxopiperidin-3-yl]-4- fluoro-1-oxo-2,3-dihydro-1H-isoindol-5- yl}-3,3-dimethylpiperidin-1- yl]cyclobutoxy]piperidin-1-yl}pyrimidin- 4-yl)amino]-2-oxo-1-(propan-2-yl)-1,2- dihydroquinolin-3-yl}oxy)-N- methylacetamide |
| 211 | 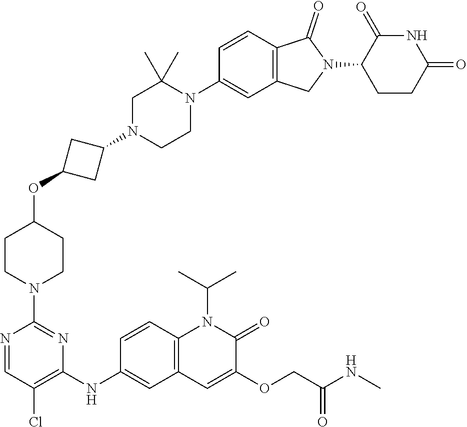 [SMILES: CNC(=O)COc1cc2cc(Nc3nc(N4CCC(O[C@H]5C[C@H](N6CCN(c7ccc8c(c7)CN([C@H]7CCC(=O)NC7=O)C8=O)C(C)(C)C6)C5)CC4)ncc3Cl)ccc2n(C(C)C)c1=O] | 2-({6-[(5-chloro-2-{4-[(1r,3r)-3-(4-{2- [(3S)-2,6-dioxopiperidin-3-yl]-1-oxo-2,3- dihydro-1H-isoindol-5-yl}-3,3- dimethylpiperazin-1- yl)cyclobutoxy]piperidin-1-yl}pyrimidin- 4-yl)amino]-2-oxo-1-(proapn-2-yl)-1,2- dihydroquinolin-3-yl}oxy)-N- methylacetamide |
| 212 | 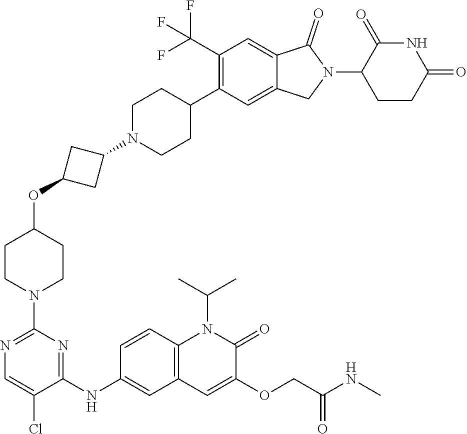 [SMILES: CNC(=O)COc1cc2cc(Nc3nc(N4CCC(O[C@H]5C[C@H](N6CCC(c7cc8c(cc7C(F)(F)F)C(=O)N(C7CCC(=O)NC7=O)C8)CC6)C5)CC4)ncc3Cl)ccc2n(C(C)C)c1=O] | 2-({6-[(5-chloro-2-{4-[(1r,3r)-3-{4-[2-(2,6- dioxopiperidin-3-yl)-1-oxo-6- (trifluoromethyl)-2,3-dihydro-1H-isoindol-5- yl]piperidin-1-yl}cyclobutoxy]piperidin-1- yl}pyrimidin-4-yl)amino]-2-oxo-1-(propan-2- yl)-1,2-dihydroquinolin-3-yl}oxy)-N- methylacetamide |
| 213 | 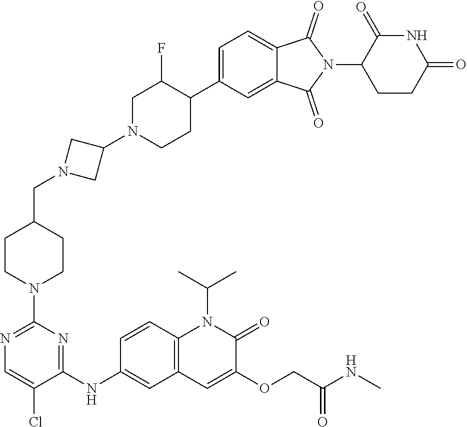 [SMILES: CNC(=O)COc1cc2cc(Nc3nc(N4CCC(CN5CC(N6CCC(c7ccc8c(c7)C(=O)N(C7CCC(=O)NC7=O)C8=O)C(F)C6)C5)CC4)ncc3Cl)ccc2n(C(C)C)c1=O] | 2-({6-[(5-chloro-2-{4-[(3-{4-[2-(2,6- dioxopiperidin-3-yl)-1,3-dioxo-2,3-dihydro- 1H-isoindol-5-yl]-3-fluoropiperidin-1- yl}azetidin-1-yl)methyl]piperidin-1- yl}pyrimidin-4-yl)amino]-2-oxo-1-(propan-2- yl)-1,2-dihydroquinolin-3-yl}oxy)-N- methylacetamide |
| 214 | 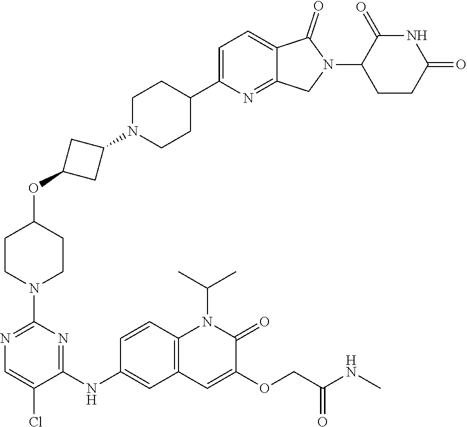 [SMILES: CNC(=O)COc1cc2cc(Nc3nc(N4CCC(O[C@H]5C[C@H](N6CCC(c7ccc8c(n7)CN(C7CCC(=O)NC7=O)C8=O)CC6)C5)CC4)ncc3Cl)ccc2n(C(C)C)c1=O] | 2-({6-[(5-chloro-2-{4-[(1r,3r)-3-{4-[6-(2,6- dioxopiperidin-3-yl)-5-oxo-5H,6H,7H- pyrrolo[3,4-b]pyridin-2-yl]piperidin-1- yl}cyclobutoxy]piperidin-1-yl}pyrimidin-4- yl)amino]-2-oxo-1-(propan-2-yl)-1,2- dihydroquinolin-3-yl}oxy)-N- methylacetamide |
| 215 | 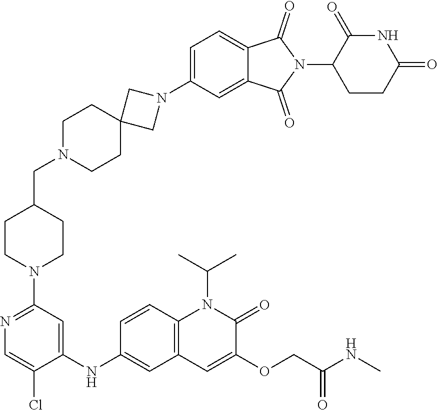 [SMILES: CNC(=O)COc1cc2cc(Nc3cc(N4CCC(CN5CCC6(CC5)CN(c5ccc7c(c5)C(=O)N(C5CCC(=O)NC5=O)C7=O)C6)CC4)ncc3Cl)ccc2n(C(C)C)c1=O] | 2-{[6-({5-chloro-2-[4-({2-[2-(2,6- dioxopiperidin-3-yl)-1,3-dioxo-2,3-dihydro- 1H-isoindol-5-yl]-2,7-diazaspiro[3.5]nonan-7- yl}methyl)piperidin-1-yl]pyridin-4- yl}amino)-2-oxo-1-(propan-2-yl)-1,2- dihydroquinolin-3-yl]oxy}-N- methylacetamide |
| 216 | 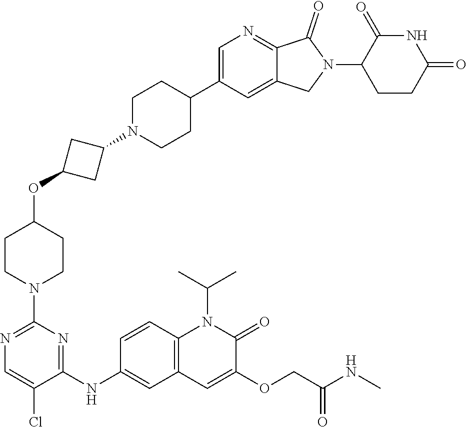 [SMILES: CNC(=O)COc1cc2cc(Nc3nc(N4CCC(O[C@H]5C[C@H](N6CCC(c7cnc8c(c7)CN(C7CCC(=O)NC7=O)C8=O)CC6)C5)CC4)ncc3Cl)ccc2n(C(C)C)c1=O] | 2-({6-[(5-chloro-2-{4-[(1r,3r)-3-{4-[6-(2,6- dioxopiperidin-3-yl)-7-oxo-5H,6H,7H- pyrrolo[3,4-b]pyridin-3-yl]piperidin-1- yl}cyclobutoxy]piepridin-1-yl}pyrimidin-4- yl)amino]-2-oxo-1-(propan-2-yl)-1,2- dihydroquinolin-3-yl}oxy)-N- methylacetamide |
| 217 | 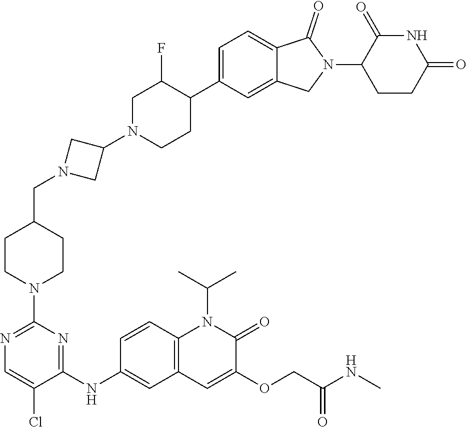 [SMILES: CNC(=O)COc1cc2cc(Nc3nc(N4CCC(CN5CC(N6CCC(c7ccc8c(c7)CN(C7CCC(=O)NC7=O)C8=O)C(F)C6)C5)CC4)ncc3Cl)ccc2n(C(C)C)c1=O] | 2-({6-[(5-chloro-2-{4-[(3-{4-[2-(2,6- dioxopiperidin-3-yl)-1-oxo-2,3-dihydro-1H- isoindol-5-yl]-3-fluoropiperidin-1-yl}azetidin- 1-yl)methyl]piperidin-1-yl}pyrimidin-4- yl)amino]-2-oxo-1-(propan-2-yl)-1,2- dihydroquinolin-3-yl}oxy)-N- methylacetamide |
| 218 | 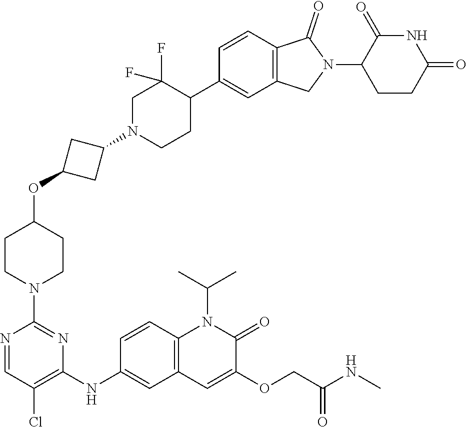 [SMILES: CNC(=O)COc1cc2cc(Nc3nc(N4CCC(O[C@H]5C[C@H](N6CCC(c7ccc8c(c7)CN(C7CCC(=O)NC7=O)C8=O)C(F)(F)C6)C5)CC4)ncc3Cl)ccc2n(C(C)C)c1=O] | 2-({6-[(5-chloro-2-{4-[(1r,3r)-3-{4-[2-(2,6- dioxopiperidin-3-yl)-1-oxo-2,3-dihydro-1H- isoindol-5-yl]-3,3-difluoropiperidin-1- yl}cyclobutoxy]piepridin-1-yl}pyrimidin-4- yl)amino]-2-oxo-1-(propan-2-yl)-1,2- dihydroquinolin-3-yl}oxy)-N- methylacetamide |
| 219 | 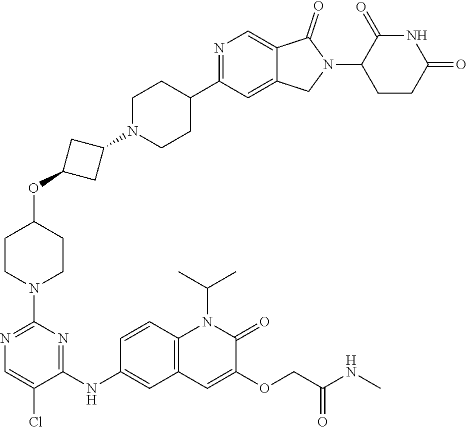 [SMILES: CNC(=O)COc1cc2cc(Nc3nc(N4CCC(O[C@H]5C[C@H](N6CCC(c7cc8c(cn7)C(=O)N(C7CCC(=O)NC7=O)C8)CC6)C5)CC4)ncc3Cl)ccc2n(C(C)C)c1=O] | 2-({6-[(5-chloro-2-{4-[(1r,3r)-3-{4-[2-(2,6- dioxopiperidin-3-yl)-3-oxo-1H,2H,3H- pyrrolo[3,4-c]pyridin-6-yl]piperidin-1- yl}cyclobutoxy]piperidin-1-yl}pyrimidin-4- yl)amino]-2-oxo-1-(propan-2-yl)-1,2- dihydroquinolin-3-yl}oxy)-N- methylacetamide |
| 220 | 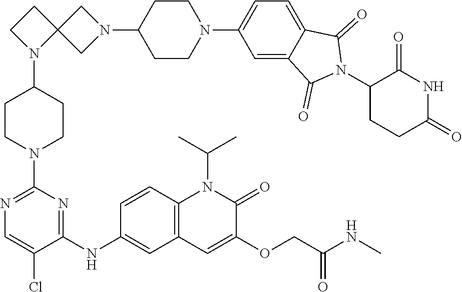 [SMILES: CNC(=O)COc1cc2cc(Nc3nc(N4CCC(N5CCC56CN(C5CCN(c7ccc8c(c7)C(=O)N(C7CCC(=O)NC7=O)C8=O)CC5)C6)CC4)ncc3Cl)ccc2n(C(C)C)c1=O] | 2-{[6-({5-chloro-2-[4-(6-{1-[2-(2,6- dioxopiperidin-3-yl)-1,3-dioxo-2,3-dihydro- 1H-isoindol-5-yl]piperidin-4-yl}-1,6- diazaspiro[3.3]heptan-1-yl)piperidin-1- yl]pyrimidin-4-yl}amino)-2-oxo-1-(propan-2- yl)-1,2-dihydroquinolin-3-yl]oxy}-N- methylacetamide |
| 221 | 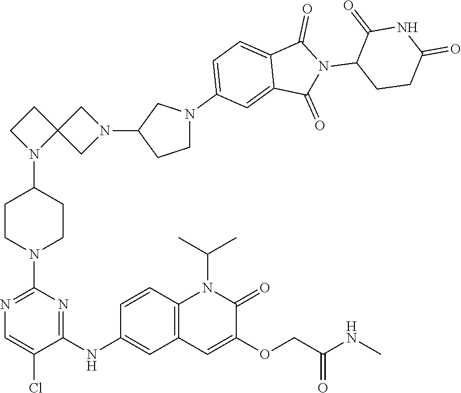 [SMILES: CNC(=O)COc1cc2cc(Nc3nc(N4CCC(N5CCC56CN(C5CCN(c7ccc8c(c7)C(=O)N(C7CCC(=O)NC7=O)C8=O)C5)C6)CC4)ncc3Cl)ccc2n(C(C)C)c1=O] | 2-{[6-({5-chloro-2-[4-(6-{1-[2-(2,6- dioxopiperidin-3-yl)-1,3-dioxo-2,3-dihydro- 1H-isoindol-5-yl]pyrrolidin-3-yl}-1,6- diazaspiro[3.3]heptan-1-yl)piperidin-1- yl]pyrimidin-4-yl}amino)-2-oxo-1-(propan-2- yl)-1,2-dihydroquinolin-3-yl]oxy}-N- methylacetamide |
| 222 | 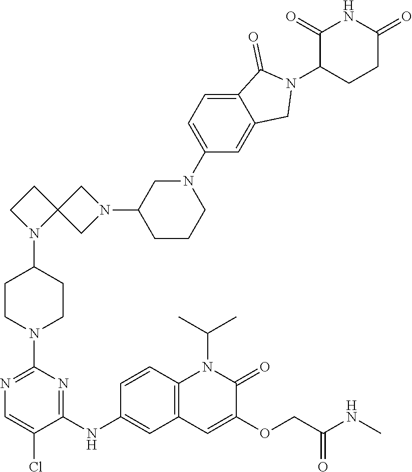 [SMILES: CNC(=O)COc1cc2cc(Nc3nc(N4CCC(N5CCC56CN(C5CCCN(c7ccc8c(c7)CN(C7CCC(=O)NC7=O)C8=O)C5)C6)CC4)ncc3Cl)ccc2n(C(C)C)c1=O] | 2-{[6-({5-chloro-2-[4-(6-{1-[2-(2,6- dioxopiperidin-3-yl)-1-oxo-2,3-dihydro-1H- isoindol-5-yl]piperidin-3-yl}-1,6- diazaspiro[3.3]heptan-1-yl)piperidin-1- yl]pyrimidin-4-yl}amino)-2-oxo-1-(propan-2- yl)-1,2-dihydroquinolin-3-yl]oxy}-N- methylacetamide |
| 223 | 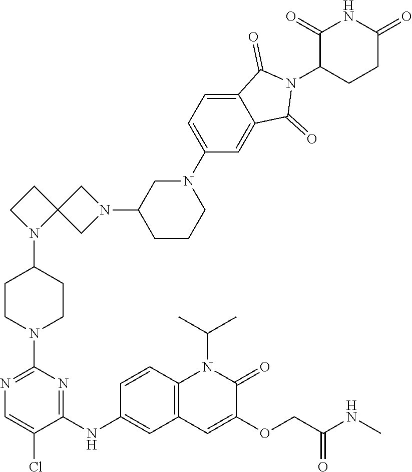 [SMILES: CNC(=O)COc1cc2cc(Nc3nc(N4CCC(N5CCC56CN(C5CCCN(c7ccc8c(c7)C(=O)N(C7CCC(=O)NC7=O)C8=O)C5)C6)CC4)ncc3Cl)ccc2n(C(C)C)c1=O] | 2-{[6-({5-chloro-2-[4-(6-{1-[2-(2,6- dioxopiperidin-3-yl)-1,3-dioxo-2,3-dihydro- 1H-isoindol-5-yl]piperidin-3-yl}-1,6- diazaspiro[3.3]heptan-1-yl)piperidin-1- yl]pyrimidin-4-yl}amino)-2-oxo-1-(propan-2- yl)-1,2-dihydroquinolin-3-yl]oxy}-N- methylacetamide |
| 224 | 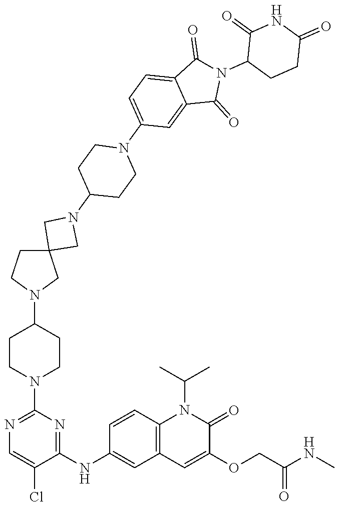 [SMILES: CNC(=O)COc1cc2cc(Nc3nc(N4CCC(N5CCC6(C5)CN(C5CCN(c7ccc8c(c7)C(=O)N(C7CCC(=O)NC7=O)C8=O)CC5)C6)CC4)ncc3Cl)ccc2n(C(C)C)c1=O] | 2-{[6-({5-chloro-2-[4-(2-{1-[2-(2,6- dioxopiperidin-3-yl)-1,3-dioxo-2,3-dihydro- 1H-isoindol-5-yl]piperidin-4-yl}-2,6- diazaspiro[3.4]octan-6-yl)piperidin-1- yl]pyrimidin-4-yl}amino)-2-oxo-1-(propan-2- yl)-1,2-dihydroquinolin-3-yl]oxy}-N- methylacetamide |
| 225 | 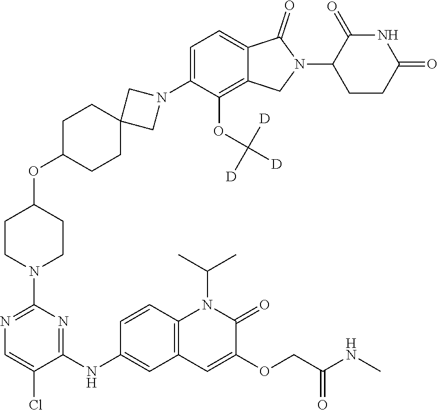 [SMILES: [2H]C([2H])([2H])Oc1c(N2CC3(CCC(OC4CCN(c5ncc(Cl)c(Nc6ccc7c(c6)cc(OCC(=O)NC)c(=O)n7C(C)C)n5)CC4)CC3)C2)ccc2c1CN(C1CCC(=O)NC1=O)C2=O] | 2-{[6-({5-chloro-2-[4-({2-[2-(2,6- dioxopiperidin-3-yl)-4-(²H₃)methoxy-1-oxo- 2,3-dihydro-1H-isoindol-5-yl]-2- azaspiro[3.5]nonan-7-yl}oxy)piperidin-1- yl]pyrimidin-4-yl}amino)-2-oxo-1-(propan-2- yl)-1,2-dihydroquinolin-3-yl]oxy}-N- methylacetamide |
| 226 | 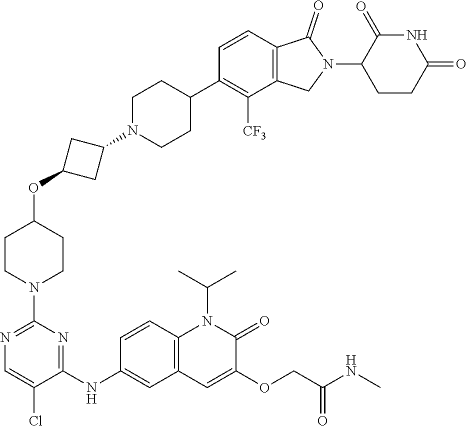 [SMILES: CNC(=O)COc1cc2cc(Nc3nc(N4CCC(O[C@H]5C[C@H](N6CCC(c7ccc8c(c7C(F)(F)F)CN(C7CCC(=O)NC7=O)C8=O)CC6)C5)CC4)ncc3Cl)ccc2n(C(C)C)c1=O] | 2-({6-[(5-chloro-2-{4-[(1r,3r)-3-{4-[2-(2,6- dioxopiperidin-3-yl)-1-oxo-4- (trifluoromethyl)-2,3-dihydro-1H-isoindol-5- yl]piperidin-1-yl}cyclobutoxy]piperidin-1- yl}pyrimidin-4-yl)amino]-2-oxo-1-(propan-2- yl)-1,2-dihydroquinolin-3-yl}oxy)-N- methylacetamide |
| 227 | 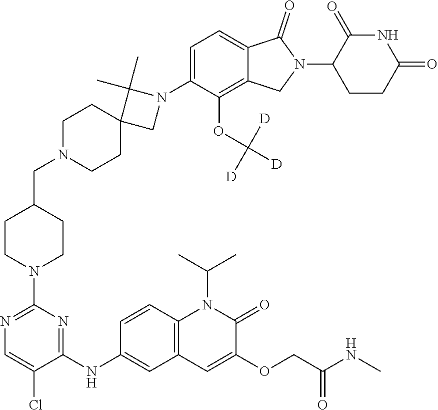 [SMILES: [2H]C([2H])([2H])Oc1c(N2CC3(CCN(CC4CCN(c5ncc(Cl)c(Nc6ccc7c(c6)cc(OCC(=O)NC)c(=O)n7C(C)C)n5)CC4)CC3)C2(C)C)ccc2c1CN(C1CCC(=O)NC1=O)C2=O] | 2-{[6-({5-chloro-2-[4-({2-[2-(2,6- dioxopiperidin-3-yl)-4-(²H₃)methoxy-1-oxo- 2,3-dihydro-1H-isoindol-5-yl]-1,1-dimethyl- 2,7-diazaspiro[3.5]nonan-7- yl}methyl)piperidin-1-yl]pyrimidin-4- yl}amino)-2-oxo-1-(propan-2-yl)-1,2- dihydroquinolin-3-yl]oxy}-N- methylacetamide |
| 228 | 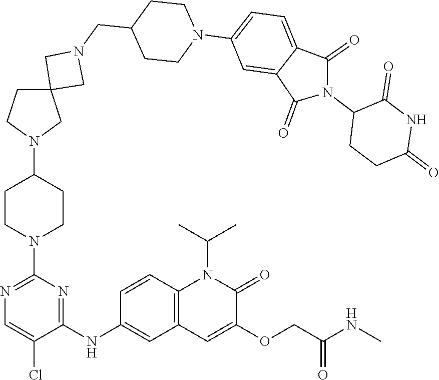 [SMILES: CNC(=O)COc1cc2cc(Nc3nc(N4CCC(N5CCC6(CN(CC7CCN(c8ccc9c(c8)C(=O)N(C8CCC(=O)NC8=O)C9=O)CC7)C6)C5)CC4)ncc3Cl)ccc2n(C(C)C)c1=O] | 2-({6-[(5-chloro-2-{4-[2-({1-[2-(2,6- dioxopiperidin-3-yl)-1,3-dioxo-2,3-dihydro- 1H-isoindol-5-yl]piperidin-4-yl}methyl)-2,6- diazaspiro[3.4]octan-6-yl]piperidin-1- yl}pyrimidin-4-yl)amino]-2-oxo-1-(propan-2- yl)-1,2-dihydroquinolin-3-yl}oxy)-N- methylacetamide |
| 229 | 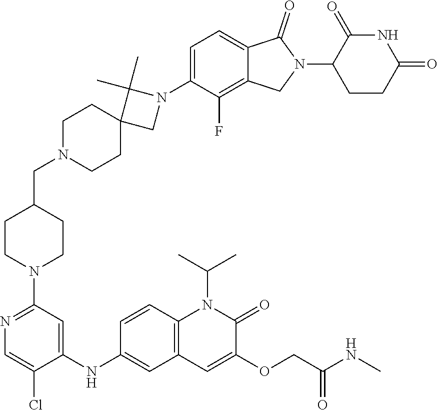 [SMILES: CNC(=O)COc1cc2cc(Nc3cc(N4CCC(CN5CCC6(CC5)CN(c5ccc7c(c5F)CN(C5CCC(=O)NC5=O)C7=O)C6(C)C)CC4)ncc3Cl)ccc2n(C(C)C)c1=O] | 2-{[6-({5-chloro-2-[4-({2-[2-(2,6- dioxopiperidin-3-yl)-4-fluoro-1-oxo-2,3- dihydro-1H-isoindol-5-yl]-1,1-dimethyl-2,7- diazaspiro[3.5]nonan-7-yl}methyl)piperidin-- 1-yl]pyridin-4-yl}amino)-2-oxo-1-(propan-2- yl)-1,2-dihydroquinolin-3-yl]oxy}-N- methylacetamide |
| 230 | 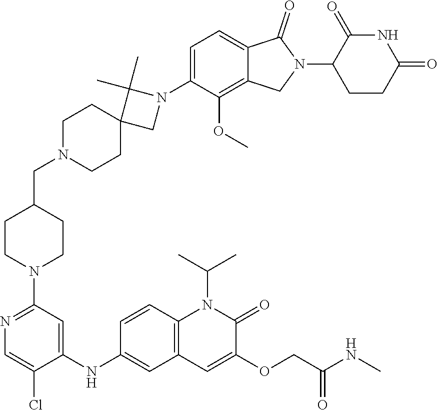 [SMILES: CNC(=O)COc1cc2cc(Nc3cc(N4CCC(CN5CCC6(CC5)CN(c5ccc7c(c5OC)CN(C5CCC(=O)NC5=O)C7=O)C6(C)C)CC4)ncc3Cl)ccc2n(C(C)C)c1=O] | 2-{[6-({5-chloro-2-[4-({2-[2-(2,6- dioxopiperidin-3-yl)-4-methoxy-1-oxo-2,3- dihydro-1H-isoindol-5-yl]-1,1-dimethyl-2,7- diazaspiro[3.5]nonan-7-yl}methyl)piperidin- 1-yl]pyridin-4-yl}amino)-2-oxo-1-(propan-2- yl)-1,2-dihydroquinolin-3-yl]oxy}-N- methylacetamide |
| 231 | 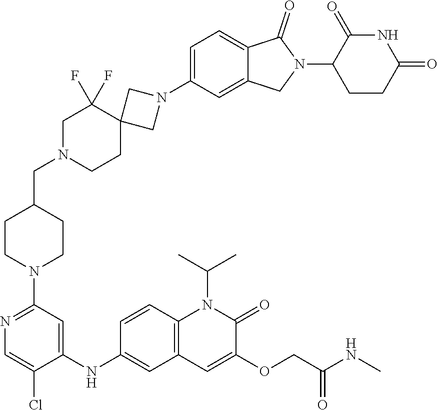 [SMILES: CNC(=O)COc1cc2cc(Nc3cc(N4CCC(CN5CCC6(CN(c7ccc8c(c7)CN(C7CCC(=O)NC7=O)C8=O)C6)C(F)(F)C5)CC4)ncc3Cl)ccc2n(C(C)C)c1=O] | 2-{[6-({5-chloro-2-[4-({2-[2-(2,6- dioxopiperidin-3-yl)-1-oxo-2,3-dihydro-1H- isoindol-5-yl]-5,5-difluoro-2,7- diazaspiro[3.5]nonan-7-yl}methyl)piperidin- 1-yl]pyridin-4-yl}amino)-2-oxo-1-(propan-2- yl)-1,2-dihydroquinolin-3-yl]oxy}-N- methylacetamide |
| 232 | 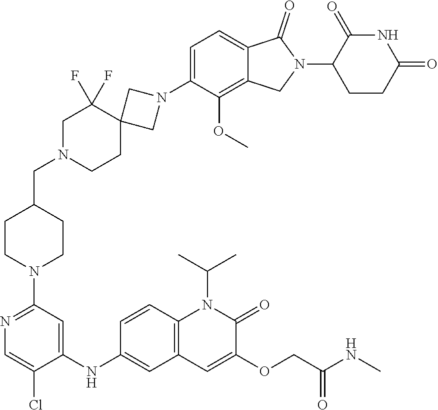 [SMILES: CNC(=O)COc1cc2cc(Nc3cc(N4CCC(CN5CCC6(CN(c7ccc8c(c7OC)CN(C7CCC(=O)NC7=O)C8=O)C6)C(F)(F)C5)CC4)ncc3Cl)ccc2n(C(C)C)c1=O] | 2-{[6-({5-chloro-2-[4-({2-[2-(2,6- dioxopiperidin-3-yl)-4-methoxy-1-oxo-2,3- dihydro-1H-isoindol-5-yl]-5,5-difluoro-2,7- diazaspiro[3.5]nonan-7-yl}methyl)piperidin- 1-yl]pyridin-4-yl}amino)-2-oxo-1-(propan-2- yl)-1,2-dihydroquinolin-3-yl]oxy}-N- methylacetamide |
| 233 | 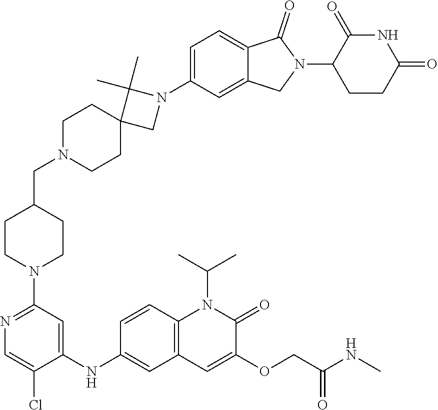 [SMILES: CNC(=O)COc1cc2cc(Nc3cc(N4CCC(CN5CCC6(CC5)CN(c5ccc7c(c5)CN(C5CCC(=O)NC5=O)C7=O)C6(C)C)CC4)ncc3Cl)ccc2n(C(C)C)c1=O] | 2-{[6-({5-chloro-2-[4-({2-[2-(2,6- dioxopiperidin-3-yl)-1-oxo-2,3-dihydro-1H- isoindol-5-yl]-1,1-dimethyl-2,7- diazaspiro[3.5]nonan-7-yl}methyl)piperidin- 1-yl]pyridin-4-yl}amino)-2-oxo-1-(propan-2- yl)-1,2-dihydroquinolin-3-yl]oxy}-N- methylactamide |
| 234 | 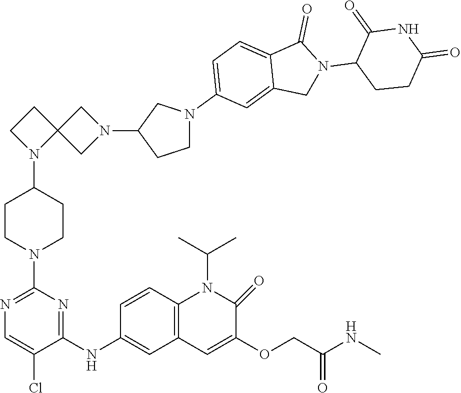 [SMILES: CNC(=O)COc1cc2cc(Nc3nc(N4CCC(N5CCC56CN(C5CCN(c7ccc8c(c7)CN(C7CCC(=O)NC7=O)C8=O)C5)C6)CC4)ncc3Cl)ccc2n(C(C)C)c1=O] | 2-{[6-({5-chloro-2-[4-(6-{1-[2-(2,6- dioxopiperidin-3-yl)-1-oxo-2,3-dihydro-1H- isoindol-5-yl]pyrrolidin-3-yl}-1,6- diazaspiro[3.3]heptan-1-yl)piperidin-1- yl]pyrimidin-4-yl}amino)-2-oxo-1-(propan-2- yl)-1,2-dihydroquinolin-3-yl]oxy}-N- methylacetamide |
| 235 | 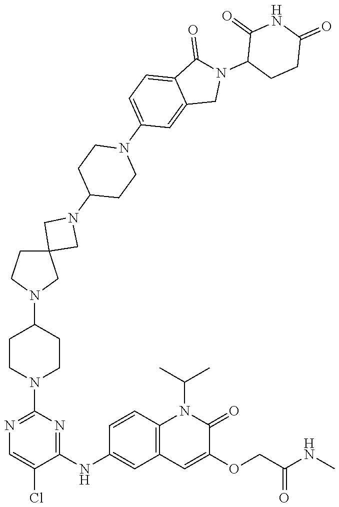 [SMILES: CNC(=O)COc1cc2cc(Nc3nc(N4CCC(N5CCC6(C5)CN(C5CCN(c7ccc8c(c7)CN(C7CCC(=O)NC7=O)C8=O)CC5)C6)CC4)ncc3Cl)ccc2n(C(C)C)c1=O] | 2-{[6-({5-chloro-2-[4-(2-{1-[2-(2,6- dioxopiperidin-3-yl)-1-oxo-2,3-dihydro-1H- isoindol-5-yl]piperidin-4-yl}-2,6- diazaspiro[3.4]octan-6-yl)piperidin-1- yl]pyrimidin-4-yl}amino)-2-oxo-1-(propan-2- yl)-1,2-dihydroquinolin-3-yl]oxy}-N- methylacetamide |
| 236 | 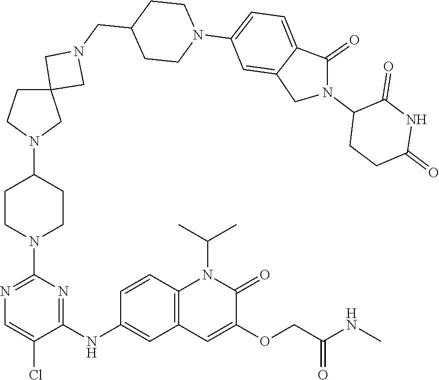 [SMILES: CNC(=O)COc1cc2cc(Nc3nc(N4CCC(N5CCC6(CN(CC7CCN(c8ccc9c(c8)CN(C8CCC(=O)NC8=O)C9=O)CC7)C6)C5)CC4)ncc3Cl)ccc2n(C(C)C)c1=O] | 2-({6-[(5-chloro-2-{4-[2-({1-[2-(2,6- dioxopiperidin-3-yl)-1-oxo-2,3-dihydro-1H- isoindol-5-yl]piperidin-4-yl}methyl)-2,6- diazaspiro[3.4]octan-6-yl]piperidin-1- yl}pyrimidin-4-yl)amino]-2-oxo-1-(propan-2- yl)-1,2-dihydroquinolin-3-yl}oxy)-N- methylacetamide |
| 237 | 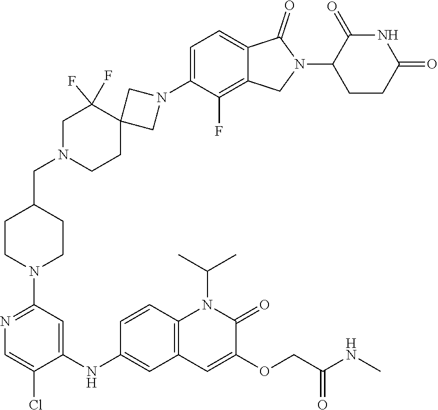 [SMILES: CNC(=O)COc1cc2cc(Nc3cc(N4CCC(CN5CCC6(CN(c7ccc8c(c7F)CN(C7CCC(=O)NC7=O)C8=O)C6)C(F)(F)C5)CC4)ncc3Cl)ccc2n(C(C)C)c1=O] | 2-{[6-({5-chloro-2-[4-({2-[2-(2,6- dioxopiperidin-3-yl)-4-fluoro-1-oxo-2,3- dihydro-1H-isoindol-5-yl]-5,5-difluoro-2,7- diazaspiro[3.5]nonan-7-yl}methyl)piperidin- 1-yl]pyridin-4-yl}amino)-2-oxo-1-(propan-2- yl)-1,2-dihydroquinolin-3-yl]oxy}-N- methylacetamide |
| 238 | 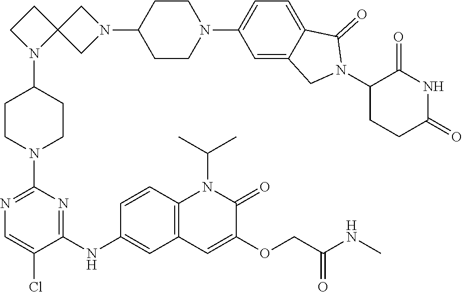 [SMILES: CNC(=O)COc1cc2cc(Nc3nc(N4CCC(N5CCC56CN(C5CCN(c7ccc8c(c7)CN(C7CCC(=O)NC7=O)C8=O)CC5)C6)CC4)ncc3Cl)ccc2n(C(C)C)c1=O] | 2-{[6-({5-chloro-2-[4-(6-{1-[2-(2,6- dioxopiperidin-3-yl)-1-oxo-2,3-dihydro-1H- isoindol-5-yl]piperidin-4-yl}-1,6- diazaspiro[3.3]heptan-1-yl)piperidin-1- yl]pyrimidin-4-yl}amino)-2-oxo-1-(propan-2- yl)-1,2-dihydroquinolin-3-yl]oxy}-N- methylacetamide |
| 239 | 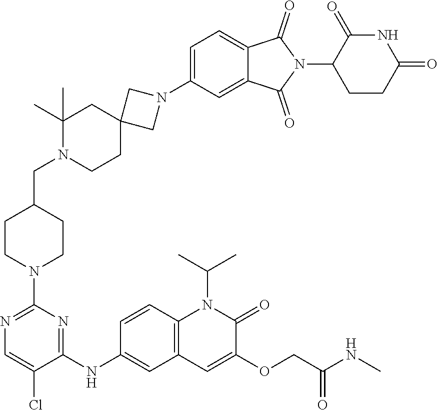 [SMILES: CNC(=O)COc1cc2cc(Nc3nc(N4CCC(CN5CCC6(CN(c7ccc8c(c7)C(=O)N(C7CCC(=O)NC7=O)C8=O)C6)CC5(C)C)CC4)ncc3Cl)ccc2n(C(C)C)c1=O] | 2-{[6-({5-chloro-2-[4-({2-[2-(2,6- dioxopiperidin-3-yl)-1,3-dioxo-2,3-dihydro- 1H-isoindol-5-yl]-6,6-dimethyl-2,7- diazaspiro[3.5]nonan-7-yl}methyl)piperidin- 1-yl]pyrimidin-4-yl}amino)-2-oxo-1-(propan- 2-yl)-1,2-dihydroquinolin-3-yl]oxy}-N- methylacetamide |
| 240 | 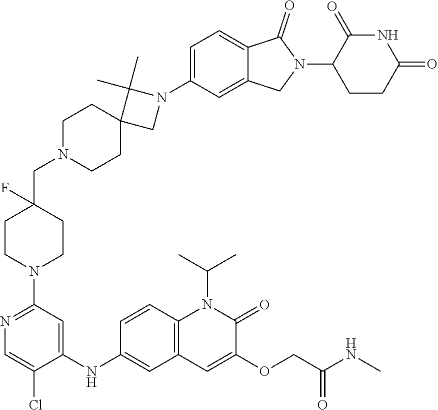 [SMILES: CNC(=O)COc1cc2cc(Nc3cc(N4CCC(F)(CN5CCC6(CC5)CN(c5ccc7c(c5)CN(C5CCC(=O)NC5=O)C7=O)C6(C)C)CC4)ncc3Cl)ccc2n(C(C)C)c1=O] | 2-{[6-({5-chloro-2-[4-({2-[2-(2,6- dioxopiperidin-3-yl)-1-oxo-2,3-dihydro-1H- isoindol-5-yl]-1,1-dimethyl-2,7- diazaspiro[3.5]nonan-7-yl}methyl)-4- fluoropiperidin-1-yl]pyridin-4-yl}amino)-2- oxo-1-(propan-2-yl)-1,2-dihydroquinolin-3- yl]oxy}-N-methylacetamide |
| 241 | 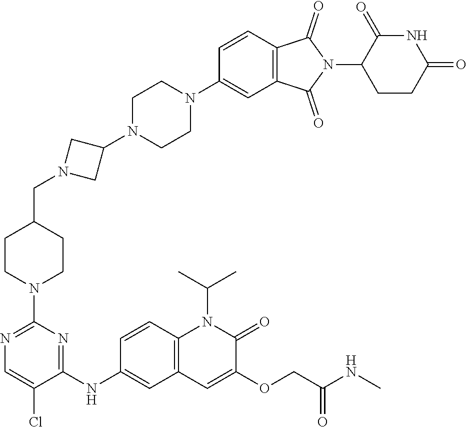 [SMILES: CNC(=O)COc1cc2cc(Nc3nc(N4CCC(CN5CC(N6CCN(c7ccc8c(c7)C(=O)N(C7CCC(=O)NC7=O)C8=O)CC6)C5)CC4)ncc3Cl)ccc2n(C(C)C)c1=O] | 2-({6-[(5-chloro-2-{4-[(3-{4-[2-(2,6- dioxopiperidin-3-yl)-1,3-dioxo-2,3-dihydro- 1H-isoindol-5-yl]piperazin-1-yl}azetidin-1- yl)methyl]piperidin-1-yl}pyrimidin-4- yl)amino]-2-oxo-1-(propan-2-yl)-1,2- dihydroquinolin-3-yl}oxy)-N- methylacetamide |
| 242 | 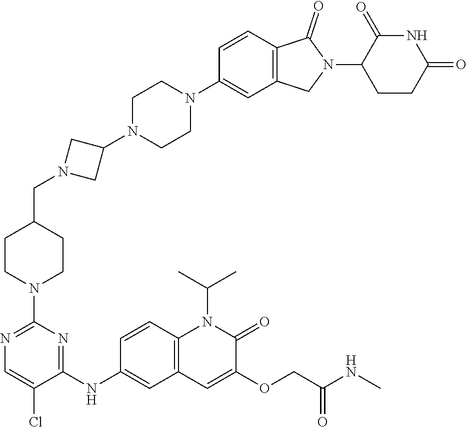 [SMILES: CNC(=O)COc1cc2cc(Nc3nc(N4CCC(CN5CC(N6CCN(c7ccc8c(c7)CN(C7CCC(=O)NC7=O)C8=O)CC6)C5)CC4)ncc3Cl)ccc2n(C(C)C)c1=O] | 2-({6-[(5-chloro-2-{4-[(3-{4-[2-(2,6- dioxopiperidin-3-yl)-1-oxo-2,3-dihydro-1H- isoindol-5-yl]piperazin-1-yl}azetidin-1- yl)methyl]piperidin-1-yl}pyrimidin-4- yl)amino]-2-oxo-1-(propan-2-yl)-1,2- dihydroquinolin-3-yl}oxy)-N- methylacetamide |
| 243 | 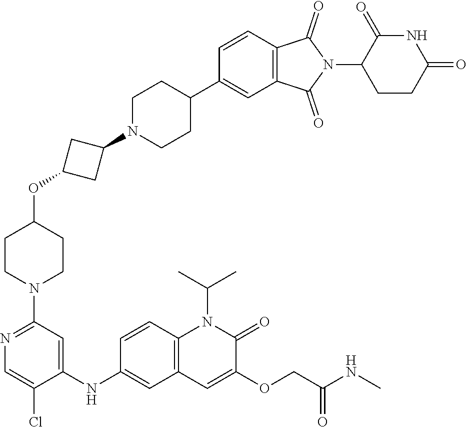 [SMILES: CNC(=O)COc1cc2cc(Nc3cc(N4CCC(O[C@H]5C[C@H](N6CCC(c7ccc8c(c7)C(=O)N(C7CCC(=O)NC7=O)C8=O)CC6)C5)CC4)ncc3Cl)ccc2n(C(C)C)c1=O] | 2-({6-[(5-chloro-2-{4-[(1r,3r)-3-{4-[2-(2,6- dioxopiperidin-3-yl)-1,3-dioxo-2,3-dihydro- 1H-isoindol-5-yl]piperidin-1- yl}cyclobutoxy]piperidin-1-yl}pyridin-4- yl)amino]-2-oxo-1-(propan-2-yl)-1,2- dihydroquinolin-3-yl}oxy)-N- methylacetamide |
| 244 | 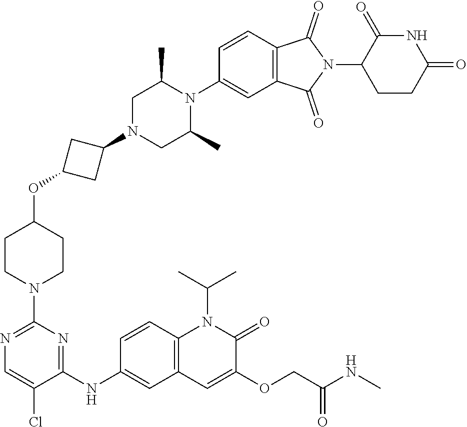 [SMILES: CNC(=O)COc1cc2cc(Nc3nc(N4CCC(O[C@H]5C[C@H](N6C[C@@H](C)N(c7ccc8c(c7)C(=O)N(C7CCC(=O)NC7=O)C8=O)[C@@H](C)C6)C5)CC4)ncc3Cl)ccc2n(C(C)C)c1=O] | 2-({6-[(5-chloro-2-{4-[(1r,3r)-3-[(3R,5S)-4- [2-(2,6-dioxopiperidin-3-yl)-1,3-dioxo-2,3- dihydro-1H-isoindol-5-yl]-3,5- dimethylpiperazin-1- yl]cyclobutoxy]piperidin-1-yl}pyrimidin-4- yl)amino]-2-oxo-1-(propan-2-yl)-1,2- dihydroquinolin-3-yl}oxy)-N- methylacetamide |
| 245 | 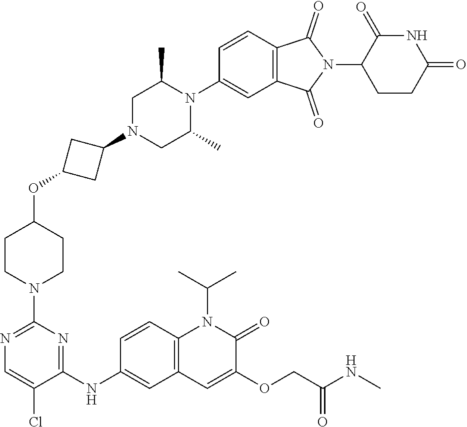 [SMILES: CNC(=O)COc1cc2cc(Nc3nc(N4CCC(O[C@H]5C[C@H](N6C[C@@H](C)N(c7ccc8c(c7)C(=O)N(C7CCC(=O)NC7=O)C8=O)[C@H](C)C6)C5)CC4)ncc3Cl)ccc2n(C(C)C)c1=O] | 2-({6-[(5-chloro-2-{4-[(1r,3r)-3-[(3R,5R)-4- [2-(2,6-dioxopiperidin-3-yl)-1,3-dioxo-2,3- dihydro-1H-isoindol-5-yl]-3,5- dimethylpiperazin-1- yl]cyclobutoxy]piperidin-1-yl}pyrimidin-4- yl)amino]-2-oxo-1-(propan-2-yl)-1,2- dihydroquinolin-3-yl}oxy)-N- methylacetamide |
| 246 | 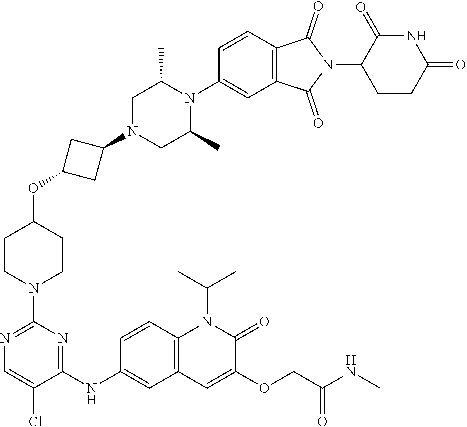 [SMILES: CNC(=O)COc1cc2cc(Nc3nc(N4CCC(O[C@H]5C[C@H](N6C[C@H](C)N(c7ccc8c(c7)C(=O)N(C7CCC(=O)NC7=O)C8=O)[C@@H](C)C6)C5)CC4)ncc3Cl)ccc2n(C(C)C)c1=O] | 2-({6-[(5-chloro-2-{4-[(1r,3r)-3-[(3S,5S)-4- [2-(2,6-dioxopiperidin-3-yl)-1,3-dioxo-2,3- dihydro-1H-isoindol-5-yl]-3,5- dimethylpiperazin-1- yl]cyclobutoxy]piperidin-1-yl}pyrimidin-4- yl)amino]-2-oxo-1-(propan-2-yl)-1,2- dihydroquinolin-3-yl}oxy)-N- methylacetamide |
| 247 | 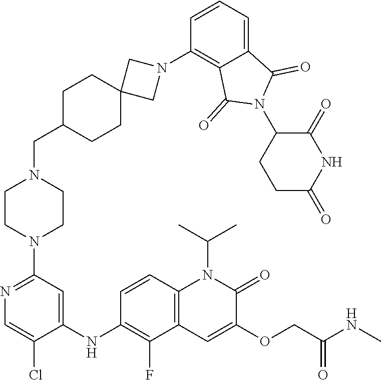 [SMILES: CNC(=O)COc1cc2c(F)c(Nc3cc(N4CCN(CC5CCC6(CC5)CN(c5cccc7c5C(=O)N(C5CCC(=O)NC5=O)C7=O)C6)CC4)ncc3Cl)ccc2n(C(C)C)c1=O] | 2-{[6-({5-chloro-2-[4-({2-[2-(2,6- dioxopiperidin-3-yl)-1,3-dioxo-2,3-dihydro- 1H-isoindol-4-yl]-2-azaspiro[3.5]nonan-7- yl}methyl)piperazin-1-yl]pyridin-4- yl}amino)-5-fluoro-2-oxo-1-(propan-2-yl)- 1,2-dihydroquinolin-3-yl]oxy}-N- methylacetamide |
| 248 | 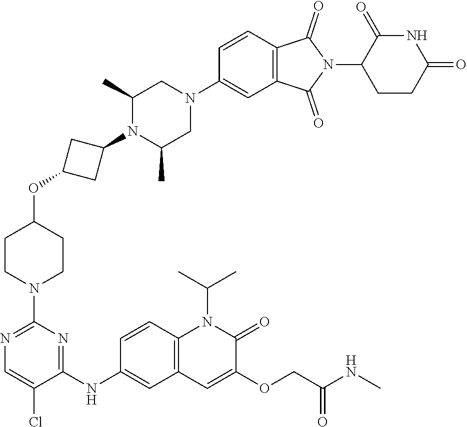 [SMILES: CNC(=O)COc1cc2cc(Nc3nc(N4CCC(O[C@H]5C[C@H](N6[C@H](C)CN(c7ccc8c(c7)C(=O)N(C7CCC(=O)NC7=O)C8=O)C[C@@H]6C)C5)CC4)ncc3Cl)ccc2n(C(C)C)c1=O] | 2-({6-[(5-chloro-2-{4-[(1r,3r)-3-[(2R,6S)-4- [2-(2,6-dioxopiperidin-3-yl)-1,3-dioxo-2,3- dihydro-1H-isoindol-5-yl]-2,6- dimethylpiperazin-1- yl]cyclobutoxy]piperidin-1-yl}pyrimidin-4- yl)amino]-2-oxo-1-(propan-2-yl)-1,2- dihydroquinolin-3-yl}oxy)-N- methylacetamide |
| 249 | 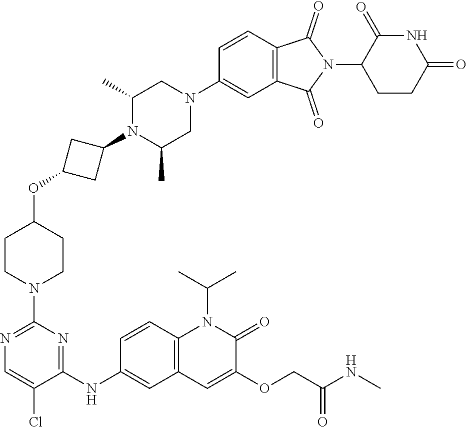 [SMILES: CNC(=O)COc1cc2cc(Nc3nc(N4CCC(O[C@H]5C[C@H](N6[C@H](C)CN(c7ccc8c(c7)C(=O)N(C7CCC(=O)NC7=O)C8=O)C[C@H]6C)C5)CC4)ncc3Cl)ccc2n(C(C)C)c1=O] | 2-({6-[(5-chloro-2-{4-[(1r,3r)-3-[(2R,6R)-4- [2-(2,6-dioxopiperidin-3-yl)-1,3-dioxo-2,3- dihydro-1H-isoindol-5-yl]-2,6- dimethylpiperazin-1- yl]cyclobutoxy]piperidin-1-yl}pyrimidin-4- yl)amino]-2-oxo-1-(propan-2-yl)-1,2- dihydroquinolin-3-yl}oxy)-N- methylacetamide |
| 250 | 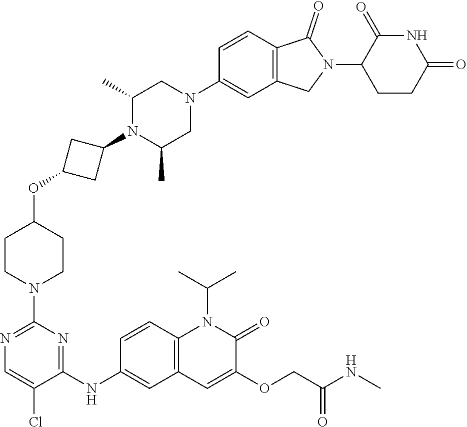 [SMILES: CNC(=O)COc1cc2cc(Nc3nc(N4CCC(O[C@H]5C[C@H](N6[C@H](C)CN(c7ccc8c(c7)CN(C7CCC(=O)NC7=O)C8=O)C[C@H]6C)C5)CC4)ncc3Cl)ccc2n(C(C)C)c1=O] | 2-({6-[(5-chloro-2-{4-[(1r,3r)-3-[(2R,6R)-4- [2-(2,6-dioxopiperidin-3-yl)-1-oxo-2,3- dihydro-1H-isoindol-5-yl]-2,6- dimethylpiperazin-1- yl]cyclobutoxy]piperidin-1-yl}pyrimidin-4- yl)amino]-2-oxo-1-(propan-2-yl)-1,2- dihydroquinolin-3-yl}oxy)-N- methylacetamide |
| 251 | 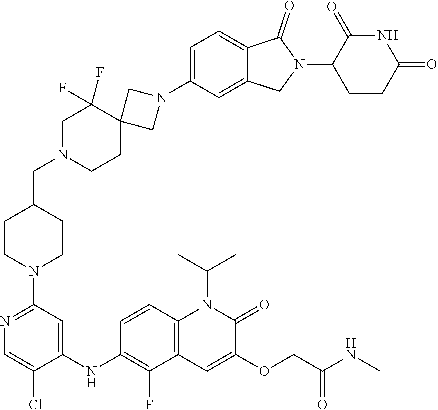 [SMILES: CNC(=O)COc1cc2c(F)c(Nc3cc(N4CCC(CN5CCC6(CN(c7ccc8c(c7)CN(C7CCC(=O)NC7=O)C8=O)C6)C(F)(F)C5)CC4)ncc3Cl)ccc2n(C(C)C)c1=O] | 2-{[6-({5-chloro-2-[4-({2-[2-(2,6- dioxopiperidin-3-yl)-1-oxo-2,3-dihydro-1H- isoindol-5-yl]-5,5-difluoro-2,7- diazaspiro[3.5]nonan-7-yl}methyl)piperidin- 1-yl]pyridin-4-yl}amino)-5-fluoro-2-oxo-1- (propan-2-yl)-1,2-dihydroquinolin-3-yl]oxy}- N-methylacetamide |
| 252 | 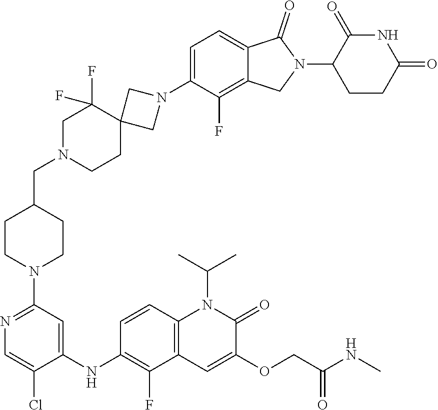 [SMILES: CNC(=O)COc1cc2c(F)c(Nc3cc(N4CCC(CN5CCC6(CN(c7ccc8c(c7F)CN(C7CCC(=O)NC7=O)C8=O)C6)C(F)(F)C5)CC4)ncc3Cl)ccc2n(C(C)C)c1=O] | 2-{[6-({5-chloro-2-[4-({2-[2-(2,6- dioxopiperidin-3-yl)-4-fluoro-1-oxo-2,3- dihydro-1H-isoindol-5-yl]-5,5-difluoro-2,7- diazaspiro[3.5]nonan-7-yl}methyl)piperidin- 1-yl]pyridin-4-yl}amino)-5-fluoro-2-oxo-1- (propan-2-yl)-1,2-dihydroquinolin-3-yl]oxy}- N-methylacetamide |
| 253 | 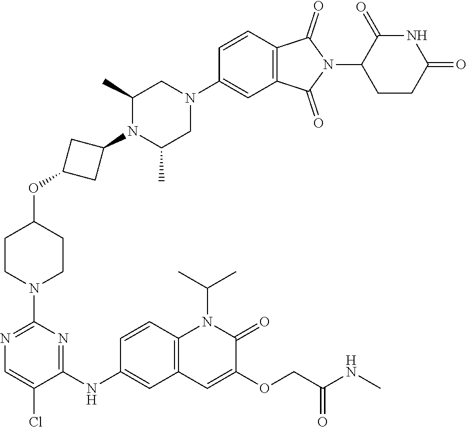 [SMILES: CNC(=O)COc1cc2cc(Nc3nc(N4CCC(O[C@H]5C[C@H](N6[C@@H](C)CN(c7ccc8c(c7)C(=O)N(C7CCC(=O)NC7=O)C8=O)C[C@@H]6C)C5)CC4)ncc3Cl)ccc2n(C(C)C)c1=O] | 2-({6-[(5-chloro-2-{4-[(1r,3r)-3-[(2S,6S)-4- [2-(2,6-dioxopiperidin-3-yl)-1,3-dioxo-2,3- dihydro-1H-isoindol-5-yl]-2,6- dimethylpiperazin-1- yl]cyclobutoxy]piperidin-1-yl}pyrimidin-4- yl)amino]-2-oxo-1-(propan-2-yl)-1,2- dihydroquinolin-3-yl}oxy)-N- methylacetamide |
| 254 | 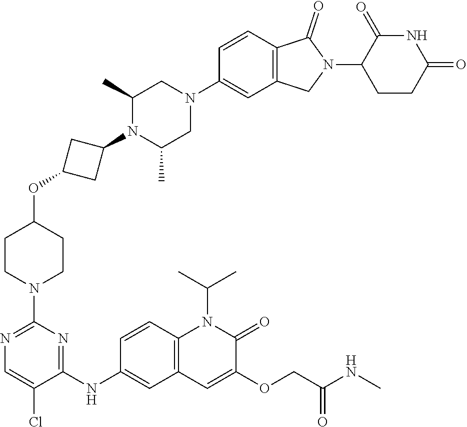 [SMILES: CNC(=O)COc1cc2cc(Nc3nc(N4CCC(O[C@H]5C[C@H](N6[C@@H](C)CN(c7ccc8c(c7)CN(C7CCC(=O)NC7=O)C8=O)C[C@@H]6C)C5)CC4)ncc3Cl)ccc2n(C(C)C)c1=O] | 2-({6-[(5-chloro-2-{4-[(1r,3r)-3-[(2S,6S)-4- [2-(2,6-dioxopiperidin-3-yl)-1-oxo-2,3- dihydro-1H-isoindol-5-yl]-2,6- dimethylpiperazin-1- yl]cyclobutoxy]piperidin-1-yl}pyrimidin-4- yl)aminio]-2-oxo-1-(propan-2-yl)-1,2- dihydroquinolin-3-yl}oxy)-N- methylacetamide |
| 255 | 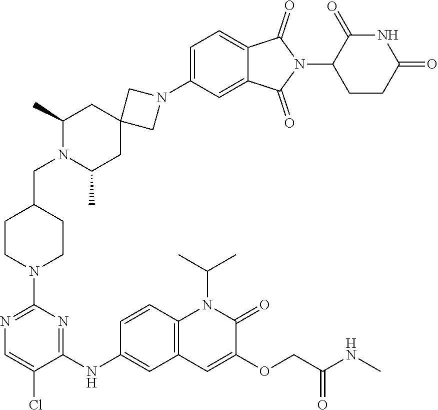 [SMILES: CNC(=O)COc1cc2cc(Nc3nc(N4CCC(CN5[C@@H](C)CC6(C[C@@H]5C)CN(c5ccc7c(c5)C(=O)N(C5CCC(=O)NC5=O)C7=O)C6)CC4)ncc3Cl)ccc2n(C(C)C)c1=O] | 2-[(6-{[5-chloro-2-(4-{[(6S,8S)-2-[2-(2,6- dioxopiperidin-3-yl)-1,3-dioxo-2,3-dihydro- 1H-isoindol-5-yl]-6,8-dimethyl-2,7- diazaspiro[3.5]nonan-7-yl]methyl}piperidin- 1-yl)pyrimidin-4-yl]amino}-2-oxo-1-(propan- 2-yl)-1,2-dihydroquinolin-3-yl)oxy]-N- methylacetamide |
| 256 | 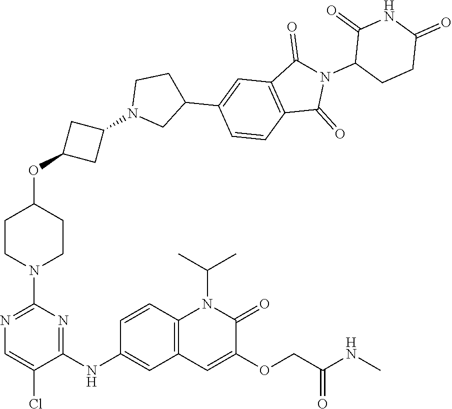 [SMILES: CNC(=O)COc1cc2cc(Nc3nc(N4CCC(O[C@H]5C[C@H](N6CCC(c7ccc8c(c7)C(=O)N(C7CCC(=O)NC7=O)C8=O)C6)C5)CC4)ncc3Cl)ccc2n(C(C)C)c1=O] | 2-({6-[(5-chloro-2-{4-[(1r,3r)-3-{3-[2-(2,6- dioxopiperidin-3-yl)-1,3-dioxo-2,3-dihydro- 1H-isoindol-5-yl]pyrrolidin-1- yl}cyclobutoxy]piperidin-1-yl}pyrimidin-4- yl)amino]-2-oxo-1-(propan-2-yl)-1,2- dihydroquinolin-3-yl}oxy)-N- methylacetamide |
| 257 | 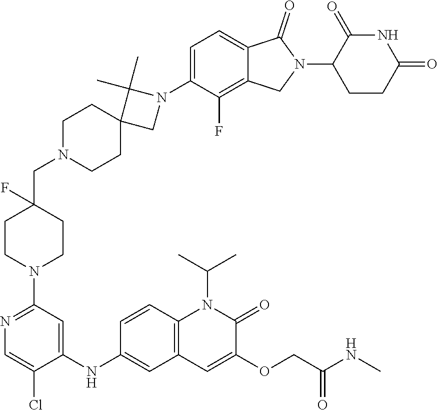 [SMILES: CNC(=O)COc1cc2cc(Nc3cc(N4CCC(F)(CN5CCC6(CC5)CN(c5ccc7c(c5F)CN(C5CCC(=O)NC5=O)C7=O)C6(C)C)CC4)ncc3Cl)ccc2n(C(C)C)c1=O] | 2-{[6-({5-chloro-2-[4-({2-[2-(2,6- dioxopiepridin-3-yl)-4-fluoro-1-oxo-2,3- dihydro-1H-isoindol-5-yl]-1,1-dimethyl-2,7- diazaspiro[3.5]nonan-7-yl}methyl)-4- fluoropiperidin-1-yl]pyridin-4-yl}amino)-2- oxo-1-(propan-2-yl)-1,2-dihydroquinolin-3- yl]oxy}-N-methylacetamide |
| 258 | 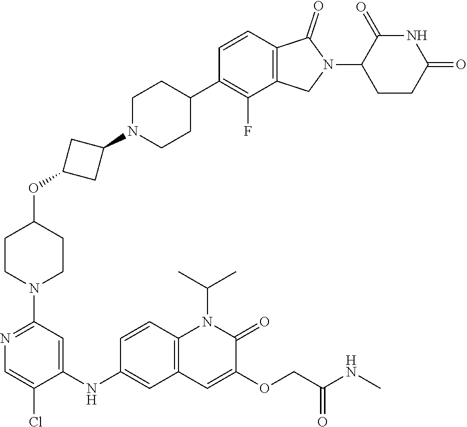 [SMILES: CNC(=O)COc1cc2cc(Nc3cc(N4CCC(O[C@H]5C[C@H](N6CCC(c7ccc8c(c7F)CN(C7CCC(=O)NC7=O)C8=O)CC6)C5)CC4)ncc3Cl)ccc2n(C(C)C)c1=O] | 2-({6-[(5-chloro-2-{4-[(1r,3r)-3-{4-[2-(2,6- dioxopiperidin-3-yl)-4-fluoro-1-oxo-2,3- dihydro-1H-isoindol-5-yl]piperidin-1- yl}cyclobutoxy]piperidin-1-yl}pyridin-4- yl)amino]-2-oxo-1-(propan-2-yl)-1,2- dihydroquinolin-3-yl}oxy)-N- methylacetamide |
| 259 | 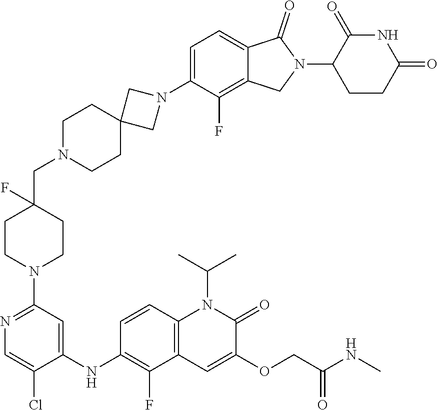 [SMILES: CNC(=O)COc1cc2c(F)c(Nc3cc(N4CCC(F)(CN5CCC6(CC5)CN(c5ccc7c(c5F)CN(C5CCC(=O)NC5=O)C7=O)C6)CC4)ncc3Cl)ccc2n(C(C)C)c1=O] | 2-{[6-({5-chloro-2-[4-({2-[2-(2,6- dioxopiperidin-3-yl)-4-fluoro-1-oxo-2,3- dihydro-1H-isoindol-5-yl]-2,7- diazaspiro[3.5]nonan-7-yl}methyl)-4- fluoropiperidin-1-yl]pyridin-4-yl}amino)-5- fluoro-2-oxo-1-(propan-2-yl)-1,2- dihydroquinolin-3-yl]oxy}-N- methylacetamide |
| 260 | 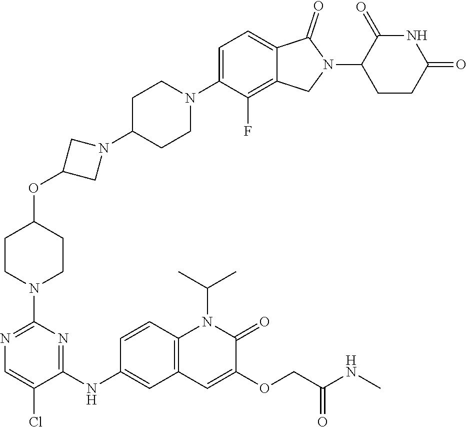 [SMILES: CNC(=O)COc1cc2cc(Nc3nc(N4CCC(OC5CN(C6CCN(c7ccc8c(c7F)CN(C7CCC(=O)NC7=O)C8=O)CC6)C5)CC4)ncc3Cl)ccc2n(C(C)C)c1=O] | 2-({6-[(5-chloro-2-{4-[(1-{1-[2-(2,6- dioxopiperidin-3-yl)-4-fluoro-1-oxo-2,3- dihydro-1H-isoindol-5-yl]piperidin-4- yl}azetidin-3-yl)oxy]piperidin-1- yl}pyrimidin-4-yl)amino]-2-oxo-1-(propan-2- yl)-1,2-dihydroquinolin-3-yl}oxy)-N- methylacetamide |
| 261 | 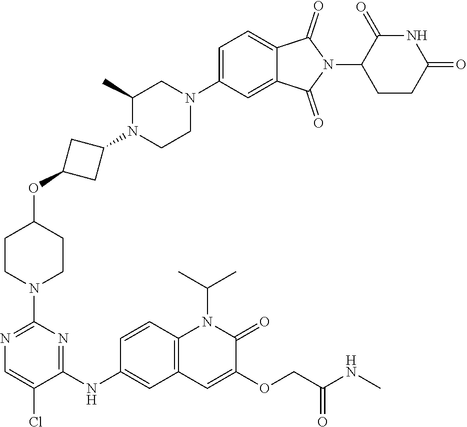 [SMILES: CNC(=O)COc1cc2cc(Nc3nc(N4CCC(O[C@H]5C[C@H](N6CCN(c7ccc8c(c7)C(=O)N(C7CCC(=O)NC7=O)C8=O)C[C@@H]6C)C5)CC4)ncc3Cl)ccc2n(C(C)C)c1=O] | 2-({6-[(5-chloro-2-{4-[(1r,3r)-3-[(2S)-4-[2- (2,6-dioxopiperidin-3-yl)-1,3-dioxo-2,3- dihydro-1H-isoindol-5-yl]-2-methylpiperazin- 1-yl]cyclobutoxy]piperidin-1-yl}pyrimidin-4- yl)amino]-2-oxo-1-(propan-2-yl)-1,2- dihydroquinolin-3-yl}oxy)-N- methylacetamide |
| 262 | 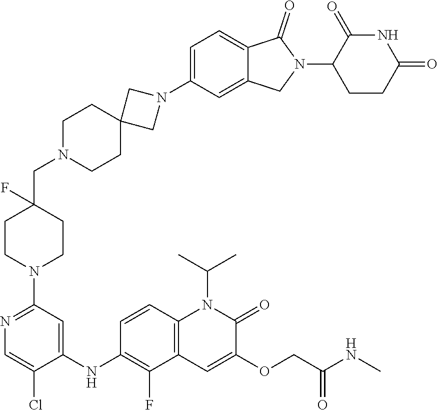 [SMILES: CNC(=O)COc1cc2c(F)c(Nc3cc(N4CCC(F)(CN5CCC6(CC5)CN(c5ccc7c(c5)CN(C5CCC(=O)NC5=O)C7=O)C6)CC4)ncc3Cl)ccc2n(C(C)C)c1=O] | 2-{[6-({5-chloro-2-[4-({2-[2-(2,6- dioxopiperidin-3-yl)-1-oxo-2,3-dihydro-1H- isoindol-5-yl]-2,7-diazaspiro[3.5]nonan-7- yl}methyl)-4-fluoropiperidin-1-yl]pyridin-4- yl}amino)-5-fluoro-2-oxo-1-(propan-2-yl)- 1,2-dihydroquinolin-3-yl]oxy}-N- methylacetamide |
| 263 | 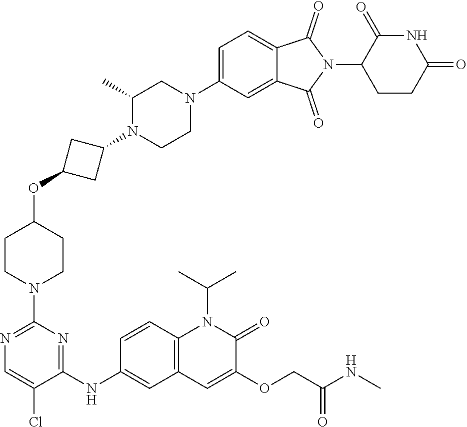 [SMILES: CNC(=O)COc1cc2cc(Nc3nc(N4CCC(O[C@H]5C[C@H](N6CCN(c7ccc8c(c7)C(=O)N(C7CCC(=O)NC7=O)C8=O)C[C@H]6C)C5)CC4)ncc3Cl)ccc2n(C(C)C)c1=O] | 2-({6-[(5-chloro-2-{4-[(1r,3r)-3-[(2R)-4-[2- (2,6-dioxopiperidin-3-yl)-1,3-dioxo-2,3- dihydro-1H-isoindol-5-yl]-2-methylpiperazin- 1-yl]cyclobutoxy]piperidin-1-yl}pyrimidin-4- yl)amino]-2-oxo-1-(proapn-2-yl)-1,2- dihydroquinolin-3-yl}oxy)-N- methylacetamide |
| 264 | 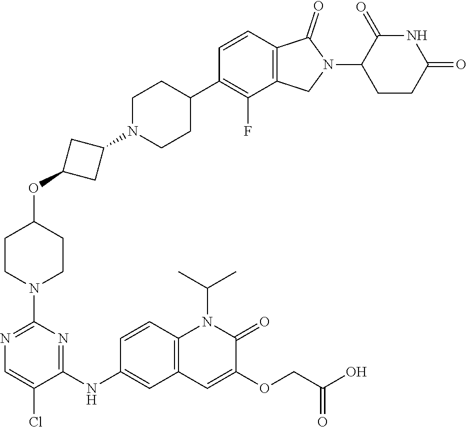 [SMILES: CC(C)n1c(=O)c(OCC(=O)O)cc2cc(Nc3nc(N4CCC(O[C@H]5C[C@H](N6CCC(c7ccc8c(c7F)CN(C7CCC(=O)NC7=O)C8=O)CC6)C5)CC4)ncc3Cl)ccc21] | 2-({6-[(5-chloro-2-{4-[(1r,3R)-3-{4-[2-(2,6- dioxopiperidin-3-yl)-4-fluoro-1-oxo-2,3- dihydro-1H-isoindol-5-yl]piperidin-1- yl}cyclobutoxy]piperidin-1-yl}pyrimidin-4- yl)amino]-2-oxo-1-(propan-2-yl)-1,2- dihydroquinolin-3-yl}oxy)acetic acid |
| 265 | 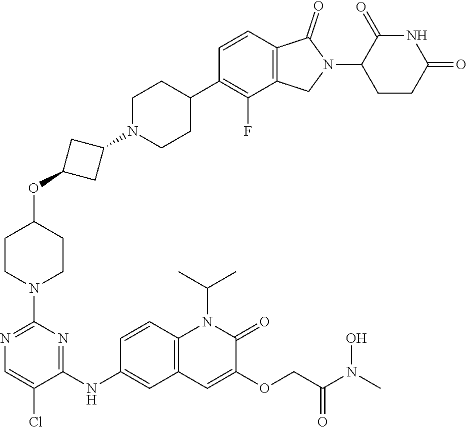 [SMILES: CC(C)n1c(=O)c(OCC(=O)N(C)O)cc2cc(Nc3nc(N4CCC(O[C@H]5C[C@H](N6CCC(c7ccc8c(c7F)CN(C7CCC(=O)NC7=O)C8=O)CC6)C5)CC4)ncc3Cl)ccc21] | 2-({6-[(5-chloro-2-{4-[(1r,3r)-3-{4-[2-(2,6- dioxopiperidin-3-yl)-4-fluoro-1-oxo-2,3- dihydro-1H-isoindol-5-yl]piperidin-1- yl}cyclobutoxy]piperidin-1-yl}pyrimidin-4- yl)amino]-2-oxo-1-(propan-2-yl)-1,2- dihydroquinolin-3-yl}oxy)-N-hydroxy-N- methylacetamide |
| 266 | 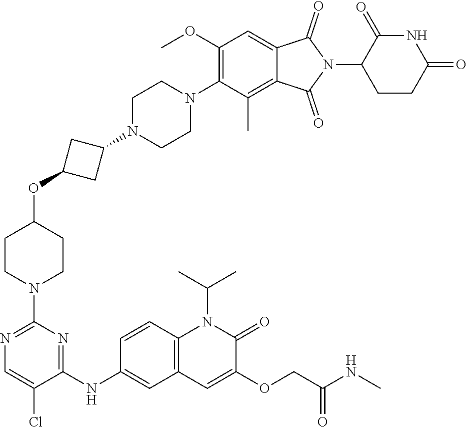 [SMILES: CNC(=O)COc1cc2cc(Nc3nc(N4CCC(O[C@H]5C[C@H](N6CCN(c7c(OC)cc8c(c7C)C(=O)N(C7CCC(=O)NC7=O)C8=O)CC6)C5)CC4)ncc3Cl)ccc2n(C(C)C)c1=O] | 2-({6-[(5-chloro-2-{4-[(1r,3r)-3-{4-[2-(2,6- dioxopiperidin-3-yl)-6-methoxy-4-methyl- 1,3-dioxo-2,3-dihydro-1H-isoindol-5- yl]piperazin-1-yl}cyclobutoxy]piperidin-1- yl}pyrimidin-4-yl)amino]-2-oxo-1-(propan-2- yl)-1,2-dihydroquinolin-3-yl}oxy)-N- methylacetamide |
| 267 | 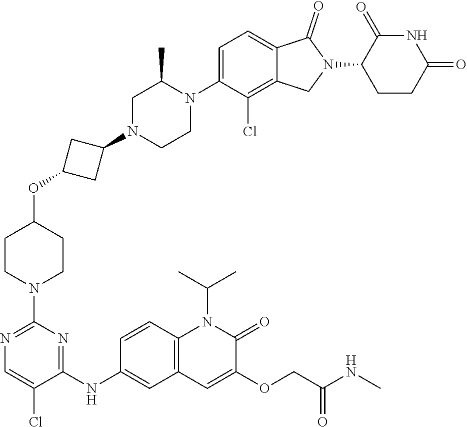 [SMILES: CNC(=O)COc1cc2cc(Nc3nc(N4CCC(O[C@H]5C[C@H](N6CCN(c7ccc8c(c7Cl)CN([C@H]7CCC(=O)NC7=O)C8=O)[C@H](C)C6)C5)CC4)ncc3Cl)ccc2n(C(C)C)c1=O] | 2-({6-[(5-chloro-2-{4-[(1r,3r)-3-[(3R)-4-{4- chloro-2-[(3S)-2,6-dioxopiperidin-3-yl]-1- oxo-2,3-dihydro-1H-isoindol-5-yl}-3- methylpiperazin-1-yl]cyclobutoxy]piperidin- 1-yl}pyrimidin-4-yl)amino]-2-oxo-1-(propan- 2-yl)-1,2-dihydroquinolin-3-yl}oxy)-N- methylacetamide |
| 268 | 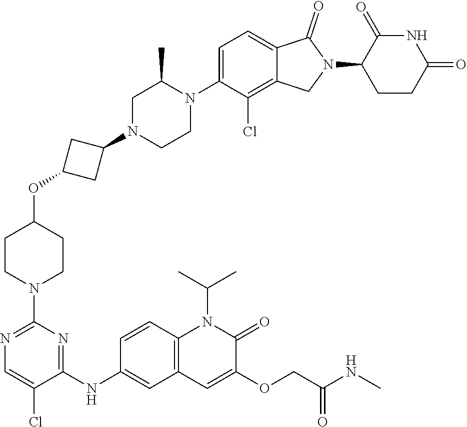 [SMILES: CNC(=O)COc1cc2cc(Nc3nc(N4CCC(O[C@H]5C[C@H](N6CCN(c7ccc8c(c7Cl)CN([C@@H]7CCC(=O)NC7=O)C8=O)[C@H](C)C6)C5)CC4)ncc3Cl)ccc2n(C(C)C)c1=O] | 2-({6-[(5-chloro-2-{4-[(1r,3r)-3-[(3R)-2-{4- chloro-2-[(3R)-2,6-dioxopiperidin-3-yl]-1- oxo-2,3-dihydro-1H-isoindol-5-yl}-3- methylpiperazin-1-yl]cyclobutoxy]piperidin- 1-yl}pyrimidin-4-yl)amino]-2-oxo-1-(propan- 2-yl)-1,2-dihydroquinolin-3-yl}oxy)-N- methylacetamide |
| 269 | 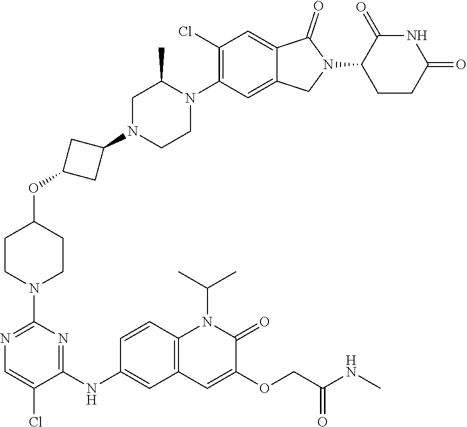 [SMILES: CNC(=O)COc1cc2cc(Nc3nc(N4CCC(O[C@H]5C[C@H](N6CCN(c7cc8c(cc7Cl)C(=O)N([C@H]7CCC(=O)NC7=O)C8)[C@H](C)C6)C5)CC4)ncc3Cl)ccc2n(C(C)C)c1=O] | 2-({6-[(5-chloro-2-{4-[(1r,3r)-3-[(3R)-4-{6- chloro-2-[(3S)-2,6-dioxopiperidin-3-yl]-1- oxo-2,3-dihydro-1H-isoindol-5-yl}-3- methylpiperazin-1-yl]cyclobutoxy]piperidn- 1-yl}pyrimidin-4-yl)amino]-2-oxo-1-(propan- 2-yl)-1,2-dihydroquinolin-3-yl}oxy)-N- methylacetamide |
| 270 | 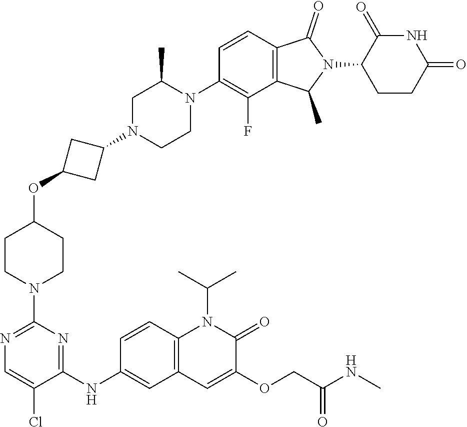 [SMILES: CNC(=O)COc1cc2cc(Nc3nc(N4CCC(O[C@H]5C[C@H](N6CCN(c7ccc8c(c7F)[C@H](C)N([C@H]7CCC(=O)NC7=O)C8=O)[C@H](C)C6)C5)CC4)ncc3Cl)ccc2n(C(C)C)c1=O] | 2-({6-[(5-chloro-2-{4-[(1r,3r)-3-[(3R)-4- [(3S)-2-[(3S)-2,6-dioxopiperidin-3-yl]-4- fluoro-3-methyl-1-oxo-2,3-dihydro-1H- isoindol-5-yl]-3-methylpiperazin-1- yl]cyclobutoxy]piperidin-1-yl}pyrimidin-4- yl)amino]-2-oxo-1-(propan-2-yl)-1,2- dihydroquinolin-3-yl}oxy)-N- methylacetamide |
| 271 | 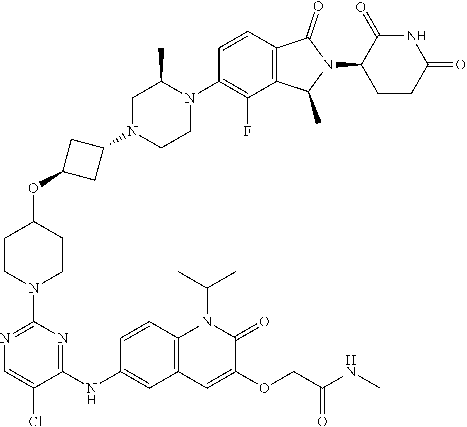 [SMILES: CNC(=O)COc1cc2cc(Nc3nc(N4CCC(O[C@H]5C[C@H](N6CCN(c7ccc8c(c7F)[C@H](C)N([C@@H]7CCC(=O)NC7=O)C8=O)[C@H](C)C6)C5)CC4)ncc3Cl)ccc2n(C(C)C)c1=O] | 2-({6-[(5-chloro-2-{4-[(1r,3r)-3-[(3R)-4- [(3S)-2-[(3R)-2,6-dioxopiperidin-3-yl]-4- fluoro-3-methyl-1-oxo-2,3-dihydro-1H- isoindol-5-yl]-3-methylpiperazin-1- yl]cyclobutoxy]piperidin-1-yl}pyrimidin-4- yl)amino]-2-oxo-1-(propan-2-yl)-1,2- dihydroquinolin-3-yl}oxy)-N- methylacetamide |
| 272 | 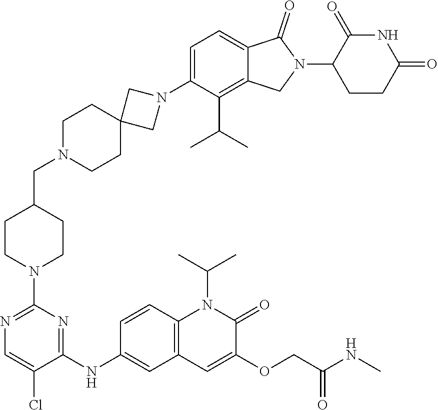 [SMILES: CNC(=O)COc1cc2cc(Nc3nc(N4CCC(CN5CCC6(CC5)CN(c5ccc7c(c5C(C)C)CN(C5CCC(=O)NC5=O)C7=O)C6)CC4)ncc3Cl)ccc2n(C(C)C)c1=O] | 2-{[6-({5-chloro-2-[4-({2-[2-(2,6- dioxopiperidin-3-yl)-1-oxo-4-(propan-2-yl)- 2,3-dihydro-1H-isoindol-5-yl]-2,7- diazaspiro[3.5]nonan-7-yl}methyl)piperidin- 1-yl]pyrimidin-4-yl}amino)-2-oxo-1-(propan- 2-yl)-1,2-dihydroquinolin-3-yl]oxy}-N- methylacetamide |
| 273 | 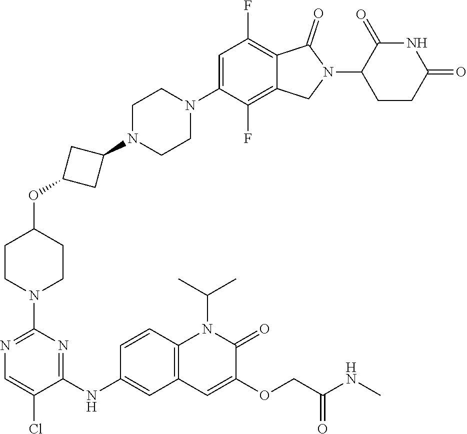 [SMILES: CNC(=O)COc1cc2cc(Nc3nc(N4CCC(O[C@H]5C[C@H](N6CCN(c7cc(F)c8c(c7F)CN(C7CCC(=O)NC7=O)C8=O)CC6)C5)CC4)ncc3Cl)ccc2n(C(C)C)c1=O] | 2-({6-[(5-chloro-2-{4-[(1r,3r)-3-{4-[2-(2,6- dioxopiperidin-3-yl)-4,7-difluoro-1-oxo-2,3- dihydro-1H-isoindol-5-yl]piperazin-1- yl}cyclobutoxy]piperidin-1-yl}pyrimidin-4- yl)amino]-2-oxo-1-(propan-2-yl)-1,2- dihydroquinolin-3-yl}oxy)-N- methylacetamide |
| 274 | 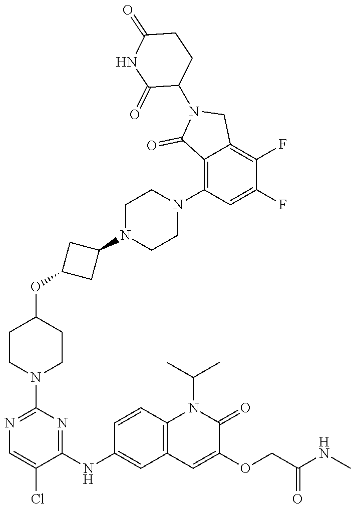 [SMILES: CNC(=O)COc1cc2cc(Nc3nc(N4CCC(O[C@H]5C[C@H](N6CCN(c7cc(F)c(F)c8c7C(=O)N(C7CCC(=O)NC7=O)C8)CC6)C5)CC4)ncc3Cl)ccc2n(C(C)C)c1=O] | 2-({6-[(5-chloro-2-{4-[(1r,3r)-3-{4-[2-(2,6- dioxopiperidin-3-yl)-6,7-difluoro-3-oxo-2,3- dihydro-1H-isoindol-4-yl]piperazin-1- yl}cyclobutoxy]piperidin-1-yl}pyrimidin-4- yl)amino]-2-oxo-1-(propan-2-yl)-1,2- dihydroquinolin-3-yl}oxy)-N- methylacetamide |
| 275 | 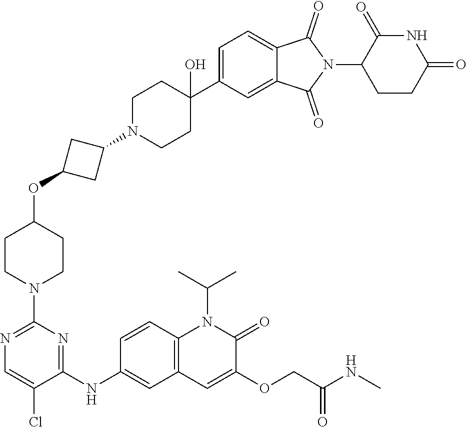 [SMILES: CNC(=O)COc1cc2cc(Nc3nc(N4CCC(O[C@H]5C[C@H](N6CCC(O)(c7ccc8c(c7)C(=O)N(C7CCC(=O)NC7=O)C8=O)CC6)C5)CC4)ncc3Cl)ccc2n(C(C)C)c1=O] | 2-({6-[(5-chloro-2-{4-[(1r,3r)-3-{4-[2-(2,6- dioxopiperidin-3-yl)-1,3-dioxo-2,3-dihydro- 1H-isoindol-5-yl]-4-hydroxypipeiridn-1- yl}cyclobutoxy]piperidin-1-yl}pyrimidin-4- yl)amino]-2-oxo-1-(propan-2-yl)-1,2- dihydroquinolin-3-yl}oxy)-N- methylacetamide |
| 276 | 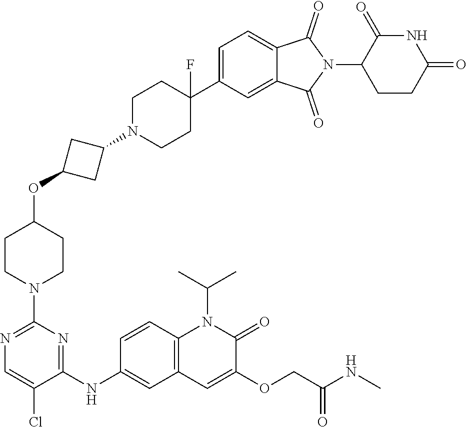 [SMILES: CNC(=O)COc1cc2cc(Nc3nc(N4CCC(O[C@H]5C[C@H](N6CCC(F)(c7ccc8c(c7)C(=O)N(C7CCC(=O)NC7=O)C8=O)CC6)C5)CC4)ncc3Cl)ccc2n(C(C)C)c1=O] | 2-({6-[(5-chloro-2-{4-[(1r,3r)-3-{4-[2-(2,6- dioxopiperidin-3-yl)-1,3-dioxo-2,3-dihydro- 1H-isoindol-5-yl]-4-fluoropiperidin-1- yl}cyclobutoxy]piperidin-1-yl}pyrimidin-4- yl)amino]-2-oxo-1-(propan-2-yl)-1,2- dihydroquinolin-3-yl}oxy)-N- methylacetamide |
| 277 | 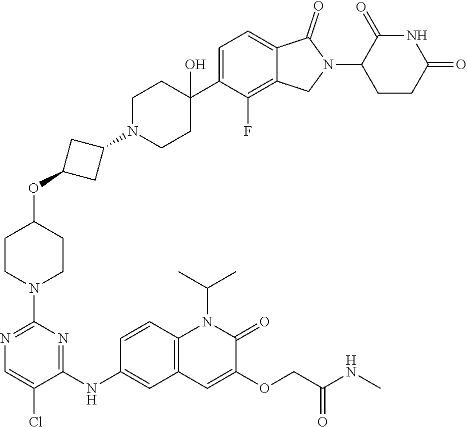 [SMILES: CNC(=O)COc1cc2cc(Nc3nc(N4CCC(O[C@H]5C[C@H](N6CCC(O)(c7ccc8c(c7F)CN(C7CCC(=O)NC7=O)C8=O)CC6)C5)CC4)ncc3Cl)ccc2n(C(C)C)c1=O] | 2-({6-[(5-chloro-2-{4-[(1r,3r)-3-{4-[2-(2,6- dioxopiperidin-3-yl)-4-fluoro-1-oxo-2,3- dihydro-1H-isoindol-5-yl]-4- hydroxypiperidin-1-yl}cyclobutoxy]piperidin- 1-yl}pyrimidin-4-yl)amino]-2-oxo-1-(propan- 2-yl)-1,2-dihydroquinolin-3-yl}oxy)-N- methylacetamide |
| 278 | 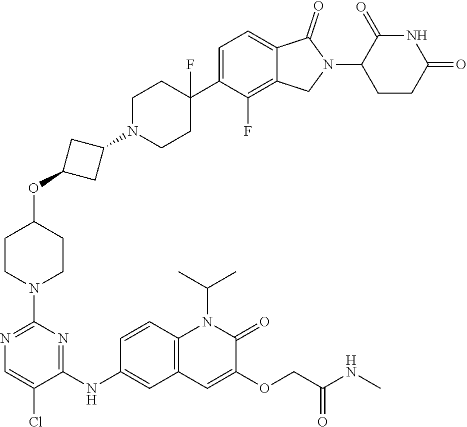 [SMILES: CNC(=O)COc1cc2cc(Nc3nc(N4CCC(O[C@H]5C[C@H](N6CCC(F)(c7ccc8c(c7F)CN(C7CCC(=O)NC7=O)C8=O)CC6)C5)CC4)ncc3Cl)ccc2n(C(C)C)c1=O] | 2-({6-[(5-chloro-2-{4-[(1r,3r)-3-{4-[2-(2,6- dioxopiperidin-3-yl)-4-fluoro-1-oxo-2,3- dihydro-1H-isoindol-5-yl]-4-fluoropiperidin- 1-yl}cyclobutoxy]piperidin-1-yl}pyrimidin-4- yl)amino]-2-oxo-1-(propan-2-yl)-1,2- dihydroquinolin-3-yl}oxy)-N- methylacetamide |
| 279 | 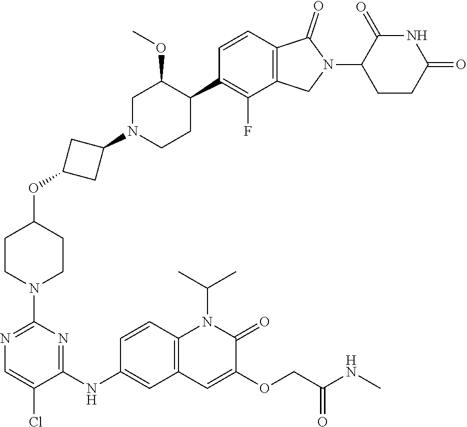 [SMILES: CNC(=O)COc1cc2cc(Nc3nc(N4CCC(O[C@H]5C[C@H](N6CC[C@H](c7ccc8c(c7F)CN(C7CCC(=O)NC7=O)C8=O)[C@H](OC)C6)C5)CC4)ncc3Cl)ccc2n(C(C)C)c1=O] | 2-({6-[(5-chloro-2-{4-[(1r,3r)-3-[(3S,4R)-4- [2-(2,6-dioxopiperidin-3-yl)-4-fluoro-1-oxo- 2,3-dihydro-1H-isoindol-5-yl]-3- methoxypiperidin-1-yl]cyclobutoxy]piperidin- 1-yl}pyrimidin-4-yl)amino]-2-oxo-1-(propan- 2-yl)-1,2-dihydroquinolin-3-yl}oxy)-N- methylacetamide |
| 280 | 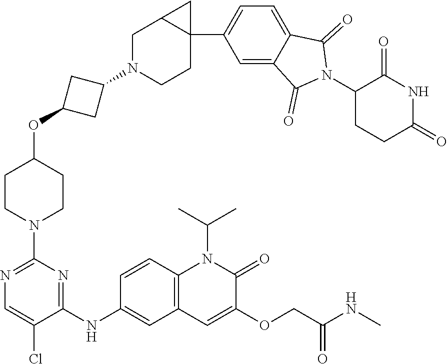 [SMILES: CNC(=O)COc1cc2cc(Nc3nc(N4CCC(O[C@H]5C[C@H](N6CCC7(c8ccc9c(c8)C(=O)N(C8CCC(=O)NC8=O)C9=O)CC7C6)C5)CC4)ncc3Cl)ccc2n(C(C)C)c1=O] | 2-({6-[(5-chloro-2-{4-[(1r,3r)-3-{6-[2-(2,6- dioxopiperidin-3-yl)-1,3-dioxo-2,3-dihydro- 1H-isoindol-5-yl]-3-azabicyclo[4.1.0]heptan- 3-yl}cyclobutoxy]piperidin-1-yl}pyrimidin-4- yl)amino]-2-oxo-1-(propan-2-yl)-1,2- dihydroquinolin-3-yl}oxy)-N- methylacetamide |
| 281 | 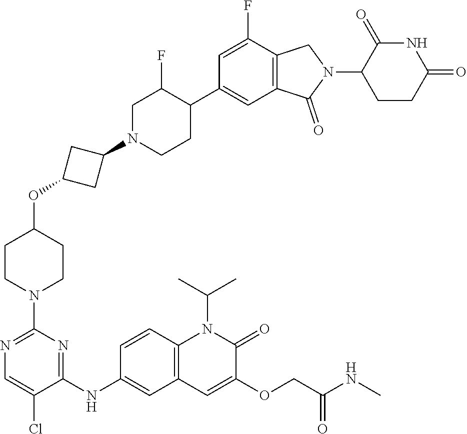 [SMILES: CNC(=O)COc1cc2cc(Nc3nc(N4CCC(O[C@H]5C[C@H](N6CCC(c7cc(F)c8c(c7)C(=O)N(C7CCC(=O)NC7=O)C8)C(F)C6)C5)CC4)ncc3Cl)ccc2n(C(C)C)c1=O] | 2-({6-[(5-chloro-2-{4-[(1r,3r)-3-{4-[2-(2,6- dioxopiperidin-3-yl)-7-fluoro-3-oxo-2,3- dihydro-1H-isoindol-5-yl]-3-fluoropiperidin- 1-yl}cyclobutoxy]piperidin-1-yl}pyrimidin-4- yl)amino]-2-oxo-1-(propan-2-yl)-1,2- dihydroquinolin-3-yl}oxy)-N- methylacetamide |
| 282 | 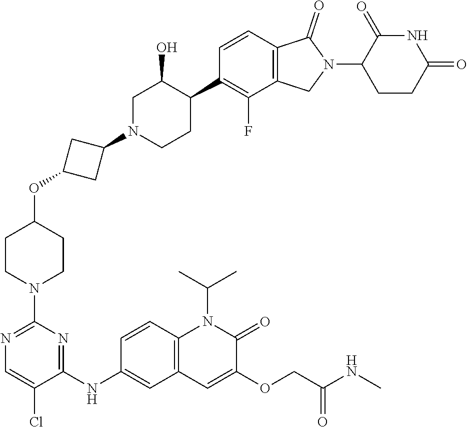 [SMILES: CNC(=O)COc1cc2cc(Nc3nc(N4CCC(O[C@H]5C[C@H](N6CC[C@H](c7ccc8c(c7F)CN(C7CCC(=O)NC7=O)C8=O)[C@H](O)C6)C5)CC4)ncc3Cl)ccc2n(C(C)C)c1=O] | 2-({6-[(5-chloro-2-{4-[(1r,3r)-3-[(3S,4R)-4- [2-(2,6-dioxopiperidin-3-yl)-4-fluoro-1-oxo- 2,3-dihydro-1H-isoindol-5-yl]-3- hydroxypriperidin-1-yl]cyclobutoxy]piperidin- 1-yl}pyrimidin-4-yl)amino]-2-oxo-1-(propan- 2-yl)-1,2-dihydroquinolin-3-yl}oxy)-N- methylacetamide |
| 283 | 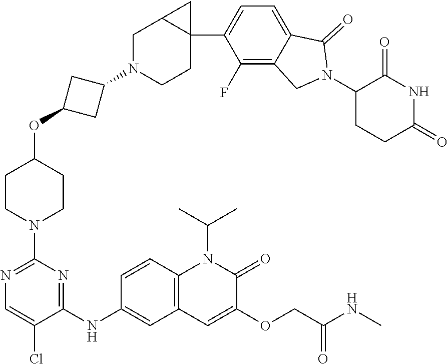 [SMILES: CNC(=O)COc1cc2cc(Nc3nc(N4CCC(O[C@H]5C[C@H](N6CCC7(c8ccc9c(c8F)CN(C8CCC(=O)NC8=O)C9=O)CC7C6)C5)CC4)ncc3Cl)ccc2n(C(C)C)c1=O] | 2-({6-[(5-chloro-2-{4-[(1r,3r)-3-{6-[2-(2,6- dioxopiperidin-3-yl)-4-fluoro-1-oxo-2,3- dihydro-1H-isoindol-5-yl]-3- azabicyclo[4.1.0]heptan-3- yl}cyclobutoxy]piperidin-1-yl}pyrimidin-4- yl)amino]-2-oxo-1-(propan-2-yl)-1,2- dihydroquinolin-3-yl}oxy)-N- methylacetamide |
| 284 | 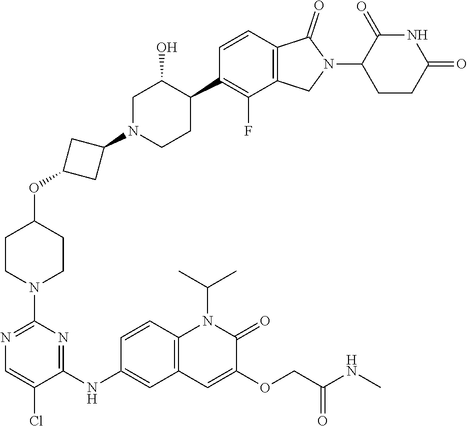 [SMILES: CNC(=O)COc1cc2cc(Nc3nc(N4CCC(O[C@H]5C[C@H](N6CC[C@H](c7ccc8c(c7F)CN(C7CCC(=O)NC7=O)C8=O)[C@@H](O)C6)C5)CC4)ncc3Cl)ccc2n(C(C)C)c1=O] | 2-({6-[(5-chloro-2-{4-[(1r,3r)-3-[(3R,4R)-4- [2-(2,6-dioxopiperidin-3-yl)-4-fluoro-1-oxo- 2,3-dihydro-1H-isoindol-5-yl]-3- hydroxypiperidin-1-yl]cyclobutoxy]piperidin- 1-yl}pyrimidin-4-yl)amino]-2-oxo-1-(propan- 2-yl)-1,2-dihydroquinolin-3-yl}oxy)-N- methylacetamide |
| 285 | 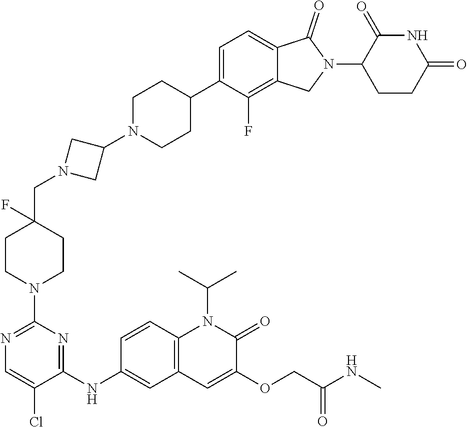 [SMILES: CNC(=O)COc1cc2cc(Nc3nc(N4CCC(F)(CN5CC(N6CCC(c7ccc8c(c7F)CN(C7CCC(=O)NC7=O)C8=O)CC6)C5)CC4)ncc3Cl)ccc2n(C(C)C)c1=O] | 2-({6-[(5-chloro-2-{4-[(3-{4-[2-(2,6- dioxopiperidin-3-yl)-4-fluoro-1-oxo-2,3- dihydro-1H-isoindol-5-yl]piperidin-1- yl}azetidin-1-yl)methyl]-4-fluoropiperidin-1- yl}pyrimidin-4-yl)amino]-2-oxo-1-(propan-2- yl)-1,2-dihydroquinolin-3-yl}oxy)-N- methylacetamide |
| 286 | 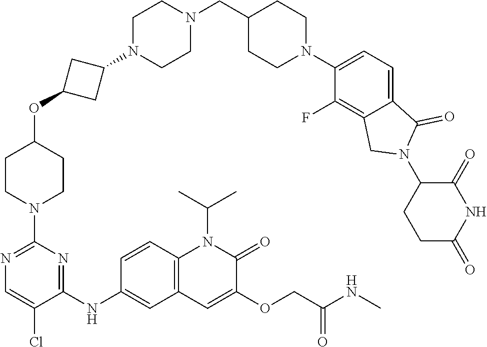 [SMILES: CNC(=O)COc1cc2cc(Nc3nc(N4CCC(O[C@H]5C[C@H](N6CCN(CC7CCN(c8ccc9c(c8F)CN(C8CCC(=O)NC8=O)C9=O)CC7)CC6)C5)CC4)ncc3Cl)ccc2n(C(C)C)c1=O] | 2-({6-[(5-chloro-2-{4-[(1r,3r)-3-[4-({1-[2- (2,6-dioxopiperidin-3-yl)-4-fluoro-1-oxo-2,3- dihdryo-1H-isoindol-5-yl]piperidin-4- yl}methyl)piperazin-1- yl]cyclobutoxy]piperidin-1-yl}pyrimidin-4- yl)amino]-2-oxo-1-(propan-2-yl)-1,2- dihydroquinolin-3-yl}oxy)-N- methylacetamide |
| 287 | 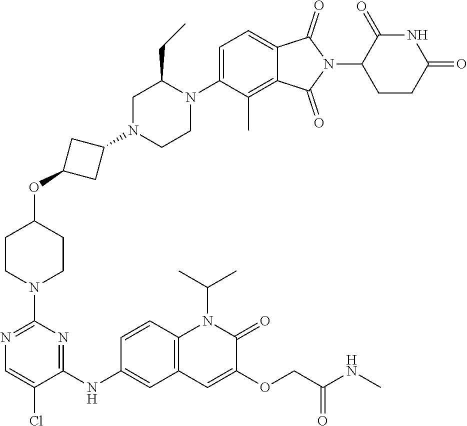 [SMILES: CC[C@@H]1CN([C@H]2C[C@H](OC3CCN(c4ncc(Cl)c(Nc5ccc6c(c5)cc(OCC(=O)NC)c(=O)n6C(C)C)n4)CC3)C2)CCN1c1ccc2c(c1C)C(=O)N(C1CCC(=O)NC1=O)C2=O] | 2-({6-[(5-chloro-2-{4-[(1r,3r)-3-[(3R)-4-[2- (2,6-dioxopiperidin-3-yl)-4-methyl-1,3-dioxo- 2,3-dihydro-1H-isoindol-5-yl]-3- ethylpiperazin-1-yl]cyclobutoxy]piperidin-1- yl}pyrimidin-4-yl)amino]-2-oxo-1-(propan-2- yl)-1,2-dihydroquinolin-3-yl}oxy)-N- methylacetamide |
| 288 | 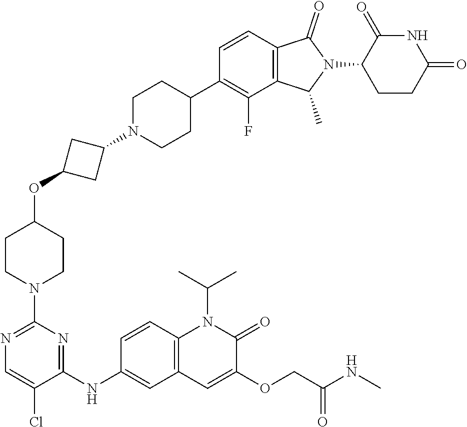 [SMILES: CNC(=O)COc1cc2cc(Nc3nc(N4CCC(O[C@H]5C[C@H](N6CCC(c7ccc8c(c7F)[C@@H](C)N([C@H]7CCC(=O)NC7=O)C8=O)CC6)C5)CC4)ncc3Cl)ccc2n(C(C)C)c1=O] | 2-({6-[(5-chloro-2-{4-[(1r,3r)-3-{4-[(3R)-2- [(3S)-2,6-dioxopiperidin-3-yl]-4-fluoro-3- methyl-1-oxo-2,3-dihydro-1H-isoindol-5- yl]piperidin-1-yl}cyclobutoxy]piperidin-1- yl}pyrimidin-4-yl)amino]-2-oxo-1-(propan-2- yl)-1,2-dihydroquinolin-3-yl}oxy)-N- methylacetamide |
| 289 | 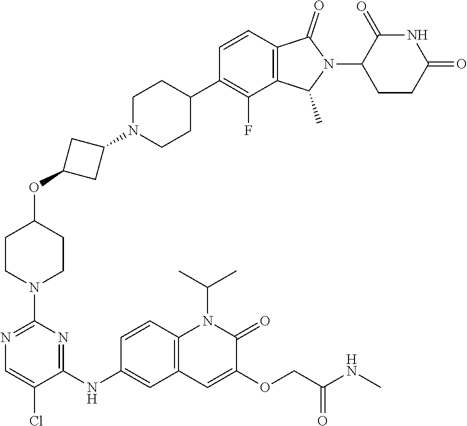 [SMILES: CNC(=O)COc1cc2cc(Nc3nc(N4CCC(O[C@H]5C[C@H](N6CCC(c7ccc8c(c7F)[C@@H](C)N(C7CCC(=O)NC7=O)C8=O)CC6)C5)CC4)ncc3Cl)ccc2n(C(C)C)c1=O] | 2-({6-[(5-chloro-2-{4-[(1r,3r)-3-{4-[(3R)-2- (2,6-dioxopiperidin-3-yl)-4-fluoro-3-methyl- 1-oxo-2,3-dihydro-1H-isoindol-5- yl]piperidin-1-yl}cyclobutoxy]piperidin-1- yl}pyrimidin-4-yl)amino]-2-oxo-1-(propan-2- yl)-1,2-dihydroquinolin-3-yl}oxy)-N- methylacetamide |
| 290 | 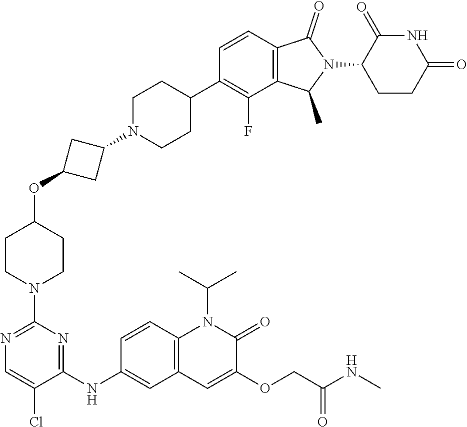 [SMILES: CNC(=O)COc1cc2cc(Nc3nc(N4CCC(O[C@H]5C[C@H](N6CCC(c7ccc8c(c7F)[C@H](C)N([C@H]7CCC(=O)NC7=O)C8=O)CC6)C5)CC4)ncc3Cl)ccc2n(C(C)C)c1=O] | 2-({6-[(5-chloro-2-{4-[(1r,3r)-3-{4-[(3S)-2- [(3S)-2,6-dioxopiperidin-3-yl]-4-fluoro-3- methyl-1-oxo-2,3-dihydro-1H-isoindol-5- yl]piperidin-1-yl}cyclobutoxy]piperidin-1- yl}pyrimidin-4-yl)amino]-2-oxo-1-(propan-2- yl)-1,2-dihydroquinolin-3-yl}oxy)-N- methylacetamide |
| 291 | 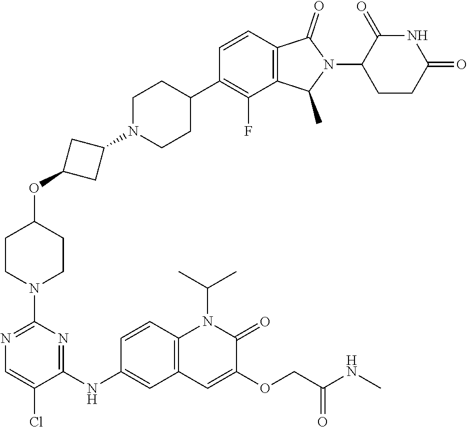 [SMILES: CNC(=O)COc1cc2cc(Nc3nc(N4CCC(O[C@H]5C[C@H](N6CCC(c7ccc8c(c7F)[C@H](C)N(C7CCC(=O)NC7=O)C8=O)CC6)C5)CC4)ncc3Cl)ccc2n(C(C)C)c1=O] | 2-({6-[(5-chloro-2-{4-[(1r,3r)-3-{4-[(3S)-2- (2,6-dioxopiperidin-3-yl)-4-fluoro-3-methyl- 1-oxo-2,3-dihydro-1H-isoindol-5- yl]piperidin-1-yl}cyclobutoxy]piperidin-1- yl}pyrimidin-4-yl)amino]-2-oxo-1-(propan-2- yl)-1,2-dihydroquinolin-3-yl}oxy)-N- methylacetamide |
| 292 | 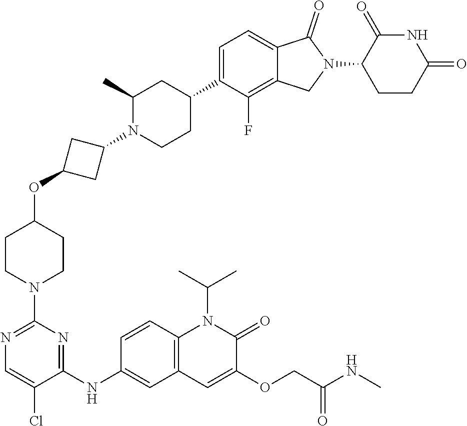 [SMILES: CNC(=O)COc1cc2cc(Nc3nc(N4CCC(O[C@H]5C[C@H](N6CC[C@@H](c7ccc8c(c7F)CN([C@H]7CCC(=O)NC7=O)C8=O)C[C@@H]6C)C5)CC4)ncc3Cl)ccc2n(C(C)C)c1=O] | 2-({6-[(5-chloro-2-{4-[(1r,3r)-3-[(2S,4R)-4- {2-[(3S)-2,6-dioxopiperidin-3-yl]-4-fluoro-1- oxo-2,3-dihydro-1H-isoindol-5-yl}-2- methylpiperidin-1-yl]cyclobutoxy]piperidin- 1-yl}pyrimidin-4-yl)amino]-2-oxo-1-(propan- 2-yl)-1,2-dihydroquinolin-3-yl}oxy)-N- methylacetamide |
| 293 | 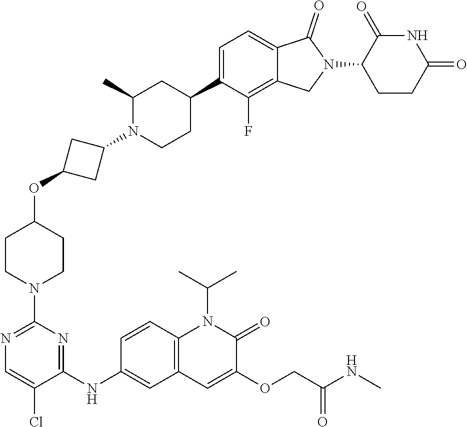 [SMILES: CNC(=O)COc1cc2cc(Nc3nc(N4CCC(O[C@H]5C[C@H](N6CC[C@H](c7ccc8c(c7F)CN([C@H]7CCC(=O)NC7=O)C8=O)C[C@@H]6C)C5)CC4)ncc3Cl)ccc2n(C(C)C)c1=O] | 2-({6-[(5-chloro-2-{4-[(1r,3r)-3-[(2S,4S)-4- {2-[(3S)-2,6-dioxopiperidin-3-yl]-4-fluoro-1- oxo-2,3-dihydro-1H-isoindol-5-yl}-2- methylpiperidin-1-yl]cyclobutoxy]piperidin- 1-yl}pyrimidin-4-yl)amino]-2-oxo-1-(propan- 2-yl)-1,2-dihydroquinolin-3-yl}oxy)-N- methylacetamide |
| 294 | 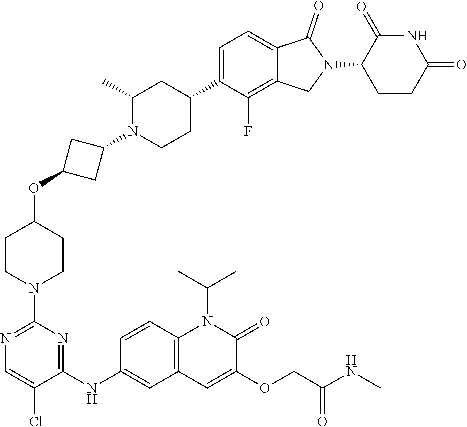 [SMILES: CNC(=O)COc1cc2cc(Nc3nc(N4CCC(O[C@H]5C[C@H](N6CC[C@@H](c7ccc8c(c7F)CN([C@H]7CCC(=O)NC7=O)C8=O)C[C@H]6C)C5)CC4)ncc3Cl)ccc2n(C(C)C)c1=O] | 2-({6-[(5-chloro-2-{4-[(1r,3r)-3-[(2R,4R)-4- {2-[(3S)-2,6-dioxopiperidin-3-yl]-4-fluoro-1- oxo-2,3-dihydro-1H-isoindol-5-yl}-2- methylpiperidin-1-yl]cyclobutoxy]piperidin- 1-yl}pyrimidin-4-yl)amino]-2-oxo-1-(propan- 2-yl)-1,2-dihydroquinolin-3-yl}oxy)-N- methylacetamide |
| 295 | 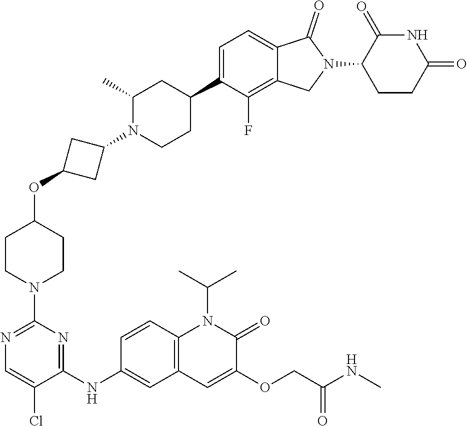 [SMILES: CNC(=O)COc1cc2cc(Nc3nc(N4CCC(O[C@H]5C[C@H](N6CC[C@H](c7ccc8c(c7F)CN([C@H]7CCC(=O)NC7=O)C8=O)C[C@H]6C)C5)CC4)ncc3Cl)ccc2n(C(C)C)c1=O] | 2-({6-[(5-chloro-2-{4-[(1r,3r)-3-[(R,4S)-4- {2-[(3S)-2,6-dioxopiperidin-3-yl]-4-fluoro-1- oxo-2,3-dihydro-1H-isoindol-5-yl}-2- methylpiperidin-1-yl]cyclobutoxy]piperidin- 1-yl}pyrimidin-4-yl)amino]-2-oxo-1-(propan- 2-yl)-1,2-dihydroquinolin-3-yl}oxy)-N- methylacetamide |
| 296 | 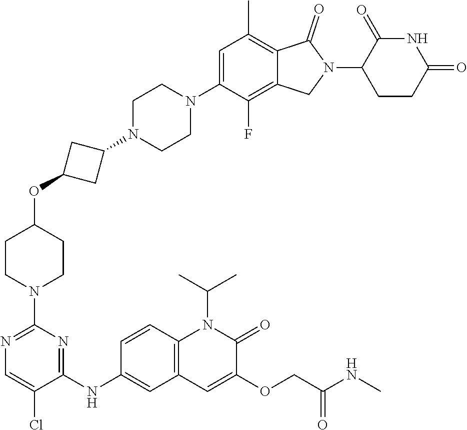 [SMILES: CNC(=O)COc1cc2cc(Nc3nc(N4CCC(O[C@H]5C[C@H](N6CCN(c7cc(C)c8c(c7F)CN(C7CCC(=O)NC7=O)C8=O)CC6)C5)CC4)ncc3Cl)ccc2n(C(C)C)c1=O] | 2-({6-[(5-chloro-2-{4-[(1r,3r)-3-{4-[2-(2,6- dioxopiperidin-3-yl)-4-fluoro-7-methyl-1- oxo-2,3-dihydro-1H-isoindol-5-yl]piperazin- 1-yl}cyclobutoxy]piperidin-1-yl}pyrimidin-4- yl)amino]-2-oxo-1-(propan-2-yl)-1,2- dihydroquinolin-3-yl}oxy)-N- methylacetamide |
| 297 | 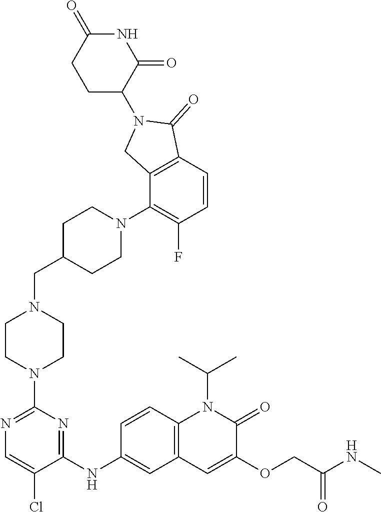 [SMILES: CNC(=O)COc1cc2cc(Nc3nc(N4CCN(CC5CCN(c6c(F)ccc7c6CN(C6CCC(=O)NC6=O)C7=O)CC5)CC4)ncc3Cl)ccc2n(C(C)C)c1=O] | 2-{6-({5-chloro-2-[4-({1-[2-(2,6- dioxopiperidin-3-yl)-5-fluoro-1-oxo-2,3- dihydro-1H-isoindol-4-yl]piperidin-4- yl}methyl)piperazin-1-yl]pyrimdiin-4- yl}amino)-2-oxo-1-(propan-2-yl)-1,2- dihydroquinolin-3-yl]oxy}-N- methylacetamide |
| 298 | 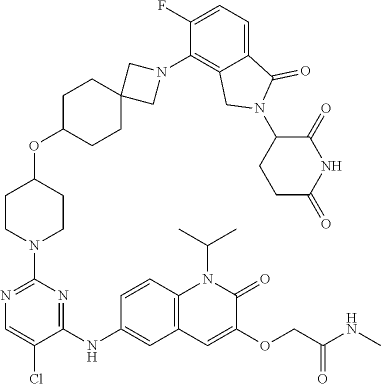 [SMILES: CNC(=O)COc1cc2cc(Nc3nc(N4CCC(OC5CCC6(CC5)CN(c5c(F)ccc7c5CN(C5CCC(=O)NC5=O)C7=O)C6)CC4)ncc3Cl)ccc2n(C(C)C)c1=O] | 2-{[6-({5-chloro-2-[4-({2-[2-(2,6- dioxopiperidin-3-yl)-5-fluoro-1-oxo-2,3- dihydro-1H-isoindol-4-yl]-2- azaspiro[3.5]nonan-7-yl}oxy)piperidin-1- yl]pyrimidin-4-yl}amino)-2-oxo-1-(propan-2- yl)-1,2-dihydroquinolin-3-yl]oxy}-N- methylacetamide |
| 299 | 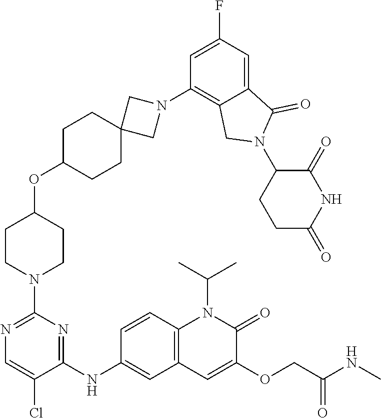 [SMILES: CNC(=O)COc1cc2cc(Nc3nc(N4CCC(OC5CCC6(CC5)CN(c5cc(F)cc7c5CN(C5CCC(=O)NC5=O)C7=O)C6)CC4)ncc3Cl)ccc2n(C(C)C)c1=O] | 2-{[6-({5-chloro-2-[4-({2-[2-(2,6- dioxopiperidin-3-yl)-6-fluoro-1-oxo-2,3- dihydro-1H-isoindol-4-yl]-2- azaspiro[3.5]nonan-7-yl}oxy)piperidin-1- yl]pyrimidin-4-yl}amino)-2-oxo-1-(propan-2- yl)-1,2-dihydroquinolin-3-yl]oxy}-N- methylacetamide |
| 300 | 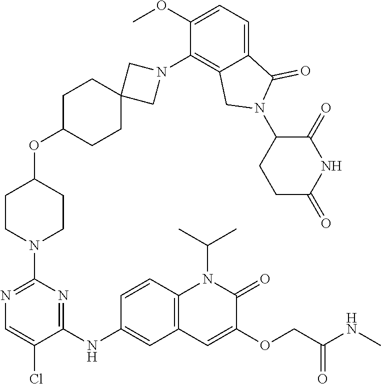 [SMILES: CNC(=O)COc1cc2cc(Nc3nc(N4CCC(OC5CCC6(CC5)CN(c5c(OC)ccc7c5CN(C5CCC(=O)NC5=O)C7=O)C6)CC4)ncc3Cl)ccc2n(C(C)C)c1=O] | 2-{[6-({5-chloro-2-[4-({2-[2-(2,6- dioxopiperidin-3-yl)-5-methoxy-1-oxo-2,3- dihydro-1H-isoindol-4-yl]-2- azaspiro[3.5]nonan-7-yl}oxy)piperidin-1- yl]pyrimidin-4-yl}amino)-2-oxo-1-(propan-2- yl)-1,2-dihydroquinolin-3-yl]oxy}-N- methylacetamide |
| 301 | 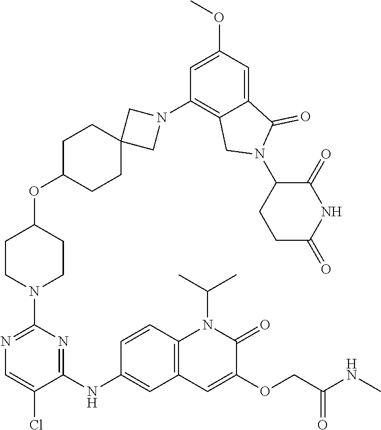 [SMILES: CNC(=O)COc1cc2cc(Nc3nc(N4CCC(OC5CCC6(CC5)CN(c5cc(OC)cc7c5CN(C5CCC(=O)NC5=O)C7=O)C6)CC4)ncc3Cl)ccc2n(C(C)C)c1=O] | 2-{[6-({5-chloro-2-[4-({2-[2-(2,6- dioxopiperidin-3-yl)-6-methoxy-1-oxo-2,3- dihydro-1H-isoindol-4-yl]-2- azaspiro[3.5]nonan-7-yl}oxy)piperidin-1- yl]pyrimdin-4-yl}amino)-2-oxo-1-(propan-2- yl)-1,2-dihydroquinolin-3-yl]oxy}-N- methylacetamide |
| 302 | 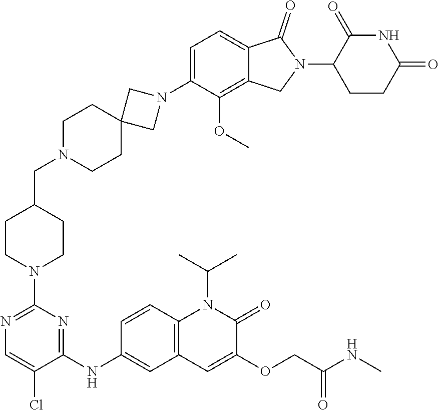 [SMILES: CNC(=O)COc1cc2cc(Nc3nc(N4CCC(CN5CCC6(CC5)CN(c5ccc7c(c5OC)CN(C5CCC(=O)NC5=O)C7=O)C6)CC4)ncc3Cl)ccc2n(C(C)C)c1=O] | 2-{[6-({5-chloro-2-[4-({2-[2-(2,6- dioxopiperidin-3-yl)-4-methoxy-1-oxo-2,3- dihydro-1H-isoindol-5-yl]-2,7- diazaspiro[3.5]nonan-7-yl}methyl)piperidin- 1-yl]pyrimidin-4-yl}amino)-2-oxo-1-(propan- 2-yl)-1,2-dihydroquinolin-3-yl]oxy}-N- methylacetamide |
| 303 | 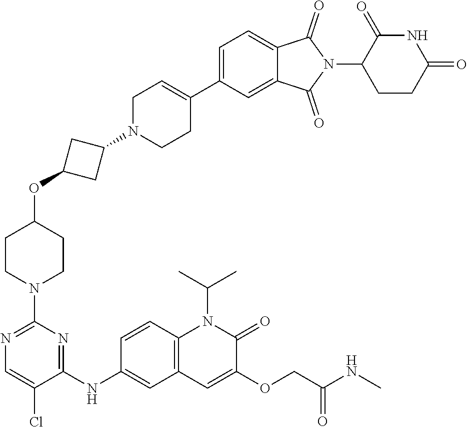 [SMILES: CNC(=O)COc1cc2cc(Nc3nc(N4CCC(O[C@H]5C[C@H](N6CC=C(c7ccc8c(c7)C(=O)N(C7CCC(=O)NC7=O)C8=O)CC6)C5)CC4)ncc3Cl)ccc2n(C(C)C)c1=O] | 2-({6-[(5-chloro-2-{4-[(1r,3r)-3-{4-[2-(2,6- dioxopiperidin-3-yl)-1,3-dioxo-2,3-dihydro- 1H-isoindol-5-yl]-1,2,3,6-tetrahydropyridin-1- yl}cyclobutoxy]piperidin-1-yl}pyrimidin-4- yl)amino]-2-oxo-1-(propan-2-yl)-1,2- dihydroquinolin-3-yl}oxy)-N- methylacetamide |
| 304 | 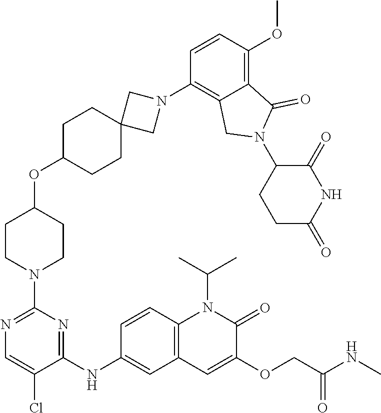 [SMILES: CNC(=O)COc1cc2cc(Nc3nc(N4CCC(OC5CCC6(CC5)CN(c5ccc(OC)c7c5CN(C5CCC(=O)NC5=O)C7=O)C6)CC4)ncc3Cl)ccc2n(C(C)C)c1=O] | 2-{[6-({5-chloro-2-[4-({2-[2-(2,6- dioxopiperidin-3-yl)-7-methoxy-1-oxo-2,3- dihydro-1H-isoindol-4-yl]-2- azaspiro[3.5]nonan-7-yl}oxy)piperidin-1- yl]pyrimidin-4-yl}amino)-2-oxo-1-(propan-2- yl)-1,2-dihydroquinolin-3-yl]oxy}-N- methylacetamide |
| 305 | 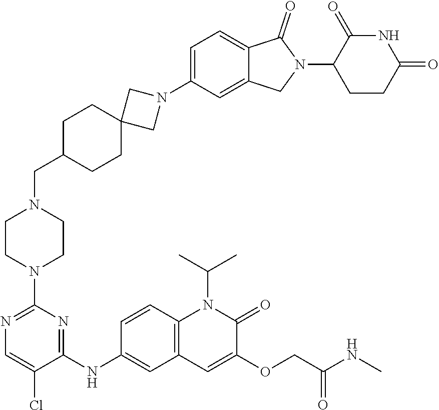 [SMILES: CNC(=O)COc1cc2cc(Nc3nc(N4CCN(CC5CCC6(CC5)CN(c5ccc7c(c5)CN(C5CCC(=O)NC5=O)C7=O)C6)CC4)ncc3Cl)ccc2n(C(C)C)c1=O] | 2-{[6-({5-chloro-2-[4-({2-[2-(2,6- dioxopiperidin-3-yl)-1-oxo-2,3-dihydro-1H- isoindol-5-yl]-2-azaspiro[3.5]nonan-7- yl}methyl)piperazin-1-yl]pyrimidin-4- yl}amino)-2-oxo-1-(propan-2-yl)-1,2- dihydroquinolin-3-yl]oxy}-N- methylacetamide |
| 306 | 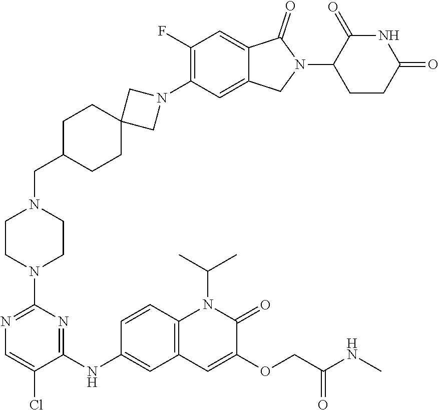 [SMILES: CNC(=O)COc1cc2cc(Nc3nc(N4CCN(CC5CCC6(CC5)CN(c5cc7c(cc5F)C(=O)N(C5CCC(=O)NC5=O)C7)C6)CC4)ncc3Cl)ccc2n(C(C)C)c1=O] | 2-{[6-({5-chloro-2-[4-({2-[2-(2,6- dioxopiperidin-3-yl)-6-fluoro-1-oxo-2,3- dihydro-1H-isoindol-5-yl]-2- azaspiro[3.5]nonan-7-yl}methyl)piperazin-1- yl]pyrimidin-4-yl}amino)-2-oxo-1-(propan-2- yl)-1,2-dihydroquinolin-3-yl]oxy}-N- methylacetamide |
| 307 | 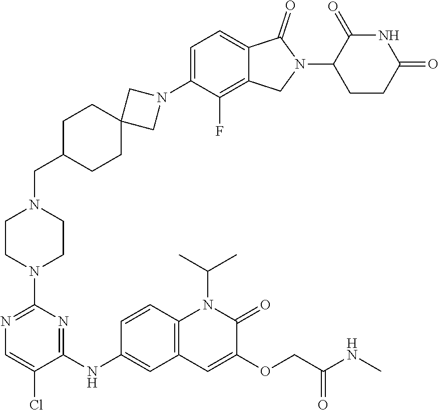 [SMILES: CNC(=O)COc1cc2cc(Nc3nc(N4CCN(CC5CCC6(CC5)CN(c5ccc7c(c5F)CN(C5CCC(=O)NC5=O)C7=O)C6)CC4)ncc3Cl)ccc2n(C(C)C)c1=O] | 2-{[6-({5-chloro-2-[4-({2-[2-(2,6- dioxopiperidin-3-yl)-4-fluoro-1-oxo-2,3- dihydro-1H-isoindol-5-yl]-2- azaspiro[3.5]nonan-7-yl}methyl)piperazin-1- yl]pyrimidin-4-yl}amino)-2-oxo-1-(propan-2- yl)-1,2-dihydroquinolin-3-yl]oxy}-N- methylacetamide |
| 308 | 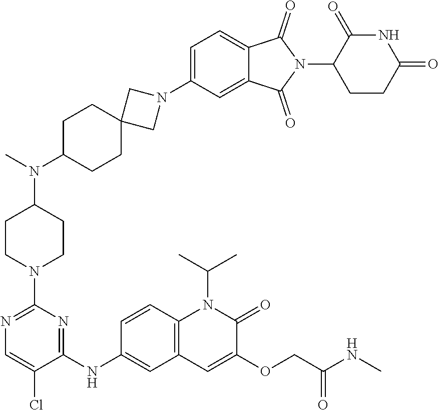 [SMILES: CNC(=O)COc1cc2cc(Nc3nc(N4CCC(N(C)C5CCC6(CC5)CN(c5ccc7c(c5)C(=O)N(C5CCC(=O)NC5=O)C7=O)C6)CC4)ncc3Cl)ccc2n(C(C)C)c1=O] | 2-{[6-({5-chloro-2-[4-({2-[2-(2,6- dioxopiperidin-3-yl)-1,3-dioxo-2,3-dihydro- 1H-isoindol-5-yl]-2-azaspiro[3.5]nonan-7- yl}(methyl)amino)piperidin-1-yl]pyrimidin-4- yl}amino)-2-oxo-1-(propan-2-yl)-1,2- dihydroquinolin-3-yl]oxy}-N- methylacetamide |
| 309 | 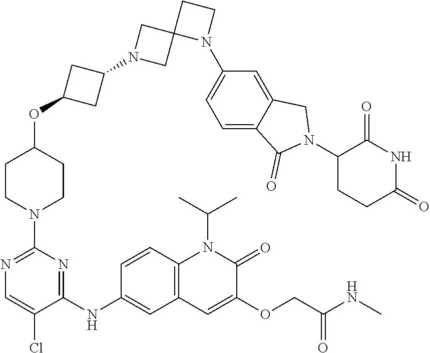 [SMILES: CNC(=O)COc1cc2cc(Nc3nc(N4CCC(O[C@H]5C[C@H](N6CC7(CCN7c7ccc8c(c7)CN(C7CCC(=O)NC7=O)C8=O)C6)C5)CC4)ncc3Cl)ccc2n(C(C)C)c1=O] | 2-({6-[(5-chloro-2-{4-[(1r,3r)-3-{1-[2-(2,6- dioxopiperidin-3-yl)-1-oxo-2,3-dihydro-1H- isoindol-5-yl]-1,6-diazaspiro[3.3]heptan-6- yl}cyclobutoxy]piperidin-1-yl}pyrimidin-4- yl)amino]-2-oxo-1-(propan-2-yl)-1,2- dihydroquinolin-3-yl}oxy)-N- methylacetamide |
| 310 | 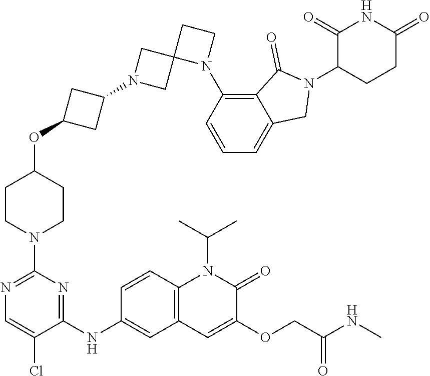 [SMILES: CNC(=O)COc1cc2cc(Nc3nc(N4CCC(O[C@H]5C[C@H](N6CC7(CCN7c7cccc8c7C(=O)N(C7CCC(=O)NC7=O)C8)C6)C5)CC4)ncc3Cl)ccc2n(C(C)C)c1=O] | 2-({6-[(5-chloro-2-{4-[(1r,3r)-3-{1-[2-(2,6- dioxopiperidin-3-yl)-3-oxo-2,3-dihydro-1H- isoindol-4-yl]-1,6-diazaspiro[3.3]heptan-6- yl}cyclobutoxy]piperidin-1-yl}pyrimidin-4- yl)amino]-2-oxo-1-(propan-2-yl)-1,2- dihydroquinolin-3-yl}oxy)-N- methylacetamide |
| 311 | 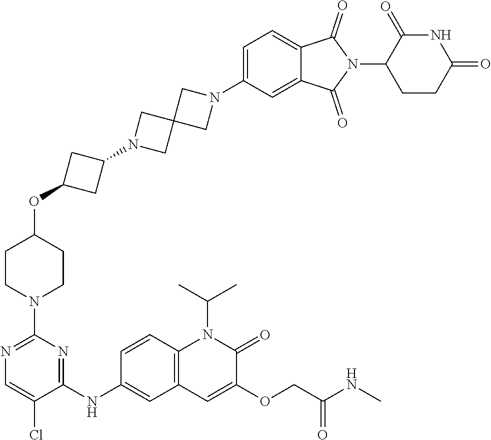 [SMILES: CNC(=O)COc1cc2cc(Nc3nc(N4CCC(O[C@H]5C[C@H](N6CC7(CN(c8ccc9c(c8)C(=O)N(C8CCC(=O)NC8=O)C9=O)C7)C6)C5)CC4)ncc3Cl)ccc2n(C(C)C)c1=O] | 2-({6-[(5-chloro-2-{4-[(1r,3r)-3-{6-[2-(2,6- dioxopiperidin-3-yl)-1,3-dioxo-2,3-dihydro- 1H-isoindol-5-yl]-2,6-diazaspiro[3.3]heptan- 2-yl}cyclobutoxy]piperidin-1-yl}pyrimidin-4- yl)amino]-2-oxo-1-(propan-2-yl)-1,2- dihydroquinolin-3-yl}oxy)-N- methylacetamide |
| 312 | 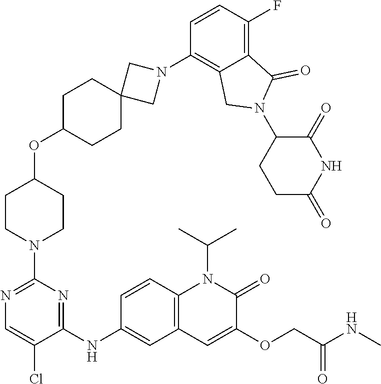 [SMILES: CNC(=O)COc1cc2cc(Nc3nc(N4CCC(OC5CCC6(CC5)CN(c5ccc(F)c7c5CN(C5CCC(=O)NC5=O)C7=O)C6)CC4)ncc3Cl)ccc2n(C(C)C)c1=O] | 2-{[6-({5-chloro-2-[4-({2-[2-(2,6- dioxopiperidin-3-yl)-7-fluoro-1-oxo-2,3- dihydro-1H-isoindol-4-yl]-2- azaspiro[3.5]nonan-7-yl}oxy)piperidin-1- yl]pyrimidin-4-yl}amino)-2-oxo-1-(propan-2- yl)-1,2-dihydroquinolin-3-yl]oxy}-N- methylacetamide |
| 313 | 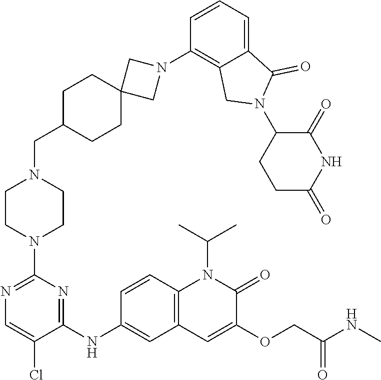 [SMILES: CNC(=O)COc1cc2cc(Nc3nc(N4CCN(CC5CCC6(CC5)CN(c5cccc7c5CN(C5CCC(=O)NC5=O)C7=O)C6)CC4)ncc3Cl)ccc2n(C(C)C)c1=O] | 2-{[6-({5-chloro-2-[4-({2-[2-(2,6- dioxopiperidin-3-yl)-1-oxo-2,3-dihydro-1H- isoindol-4-yl]-2-azaspiro[3.5]nonan-7- yl}methyl)piperazin-1-yl]pyrimidin-4- yl}amino)-2-oxo-1-(propan-2-yl)-1,2- dihydroquinolin-3-yl]oxy}-N- methylacetamide |
| 314 | 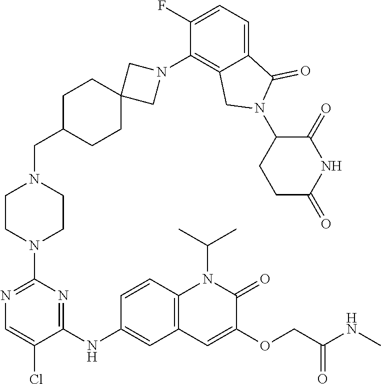 [SMILES: CNC(=O)COc1cc2cc(Nc3nc(N4CCN(CC5CCC6(CC5)CN(c5c(F)ccc7c5CN(C5CCC(=O)NC5=O)C7=O)C6)CC4)ncc3Cl)ccc2n(C(C)C)c1=O] | 2-{[6-({5-chloro-2-[4-({2-[2-(2,6- dioxopiperidin-3-yl)-5-fluoro-1-oxo-2,3- dihydro-1H-isoindol-4-yl]-2- azaspiro[3.5]nonan-7-yl}methyl)piperazin-1- yl]pyrimidin-4-yl}amino)-2-oxo-1-(propan-2- yl)-1,2-dihydroquinolin-3-yl]oxy}-N- methylacetamide |
| 315 | 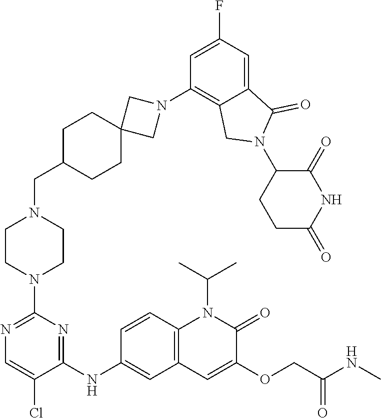 [SMILES: CNC(=O)COc1cc2cc(Nc3nc(N4CCN(CC5CCC6(CC5)CN(c5cc(F)cc7c5CN(C5CCC(=O)NC5=O)C7=O)C6)CC4)ncc3Cl)ccc2n(C(C)C)c1=O] | 2-{[6-({5-chloro-2-[4-({2-[2-(2,6- dioxopiperidin-3-yl)-6-fluoro-1-oxo-2,3- dihydro-1H-isoindol-4-yl]-2- azaspiro[3.5]nonan-7-yl}methyl)piperazin-1- yl]pyrimidin-4-yl}amino)-2-oxo-1-(propan-2- yl)-1,2-dihydroquinolin-3-yl]oxy}-N- methylacetamide |
| 316 | 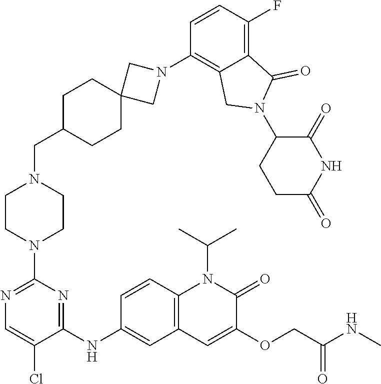 [SMILES: CNC(=O)COc1cc2cc(Nc3nc(N4CCN(CC5CCC6(CC5)CN(c5ccc(F)c7c5CN(C5CCC(=O)NC5=O)C7=O)C6)CC4)ncc3Cl)ccc2n(C(C)C)c1=O] | 2-{[6-({5-chloro-2-[4-({2-[2-(2,6- dioxopiperidin-3-yl)-7-fluoro-1-oxo-2,3- dihydro-1H-isoindol-4-yl]-2- azaspiro[3.5]nonan-7-yl}methyl)piperazin-1- yl]pyrimidin-4-yl}amino)-2-oxo-1-(propan-2- yl)-1,2-dihydroquinolin-3-yl]oxy}-N- methylacetamide |
| 317 | 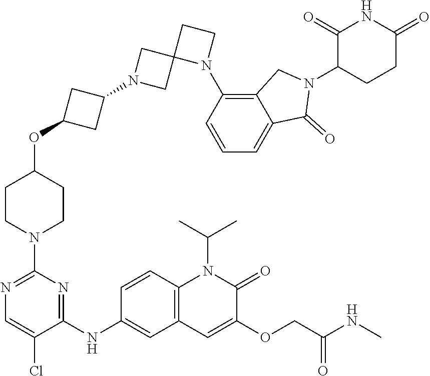 [SMILES: CNC(=O)COc1cc2cc(Nc3nc(N4CCC(O[C@H]5C[C@H](N6CC7(CCN7c7cccc8c7CN(C7CCC(=O)NC7=O)C8=O)C6)C5)CC4)ncc3Cl)ccc2n(C(C)C)c1=O] | 2-({6-[(5-chloro-2-{4-[(1r,3r)-3-{1-[2-(2,6- dioxopiperidin-3-yl)-1-oxo-2,3-dihydro-1H- isoindol-4-yl]-1,6-diazaspiro[3.3]heptan-6- yl}cyclobutoxy]piepridin-1-yl}pyrimidin-4- yl)amino]-2-oxo-1-(propan-2-yl)-1,2- dihydroquinolin-3-yl}oxy)-N- methylacetamide |
| 318 | 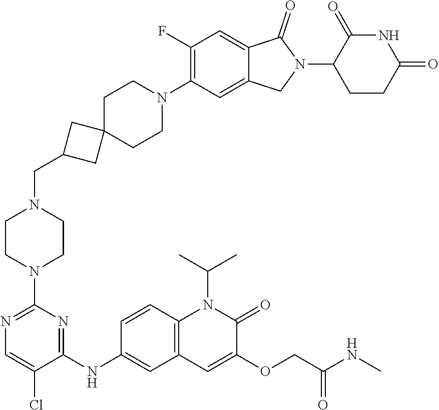 [SMILES: CNC(=O)COc1cc2cc(Nc3nc(N4CCN(CC5CC6(CCN(c7cc8c(cc7F)C(=O)N(C7CCC(=O)NC7=O)C8)CC6)C5)CC4)ncc3Cl)ccc2n(C(C)C)c1=O] | 2-{[6-({5-chloro-2-[4-({7-[2-(2,6- dioxopiperidin-3-yl)-6-fluoro-1-oxo-2,3- dihydro-1H-isoindol-5-yl]-7- azaspiro[3.5]nonan-2-yl}methyl)piperazin-1- yl]pyrimidin-4-yl}amino)-2-oxo-1-(propan-2- yl)-1,2-dihydroquinolin-3-yl]oxy}-N- methylacetamide |
| 319 | 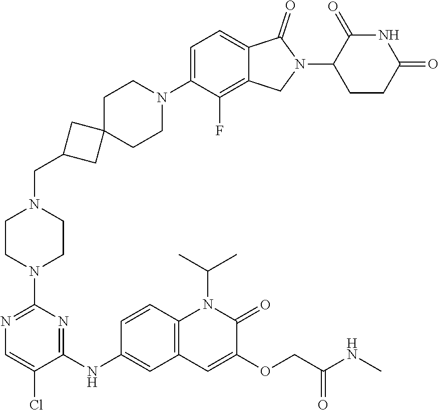 [SMILES: CNC(=O)COc1cc2cc(Nc3nc(N4CCN(CC5CC6(CCN(c7ccc8c(c7F)CN(C7CCC(=O)NC7=O)C8=O)CC6)C5)CC4)ncc3Cl)ccc2n(C(C)C)c1=O] | 2-{[6-({5-chloro-2-[4-({7-[2-(2,6- dioxopiperidin-3-yl)-4-fluoro-1-oxo-2,3- dihydro-1H-isoindol-5-yl]-7- azaspiro[3.5]nonan-2-yl}methyl)piperazin-1- yl]pyrimidin-4-yl}amino)-2-oxo-1-(propan-2- yl)-1,2-dihydroquinolin-3-yl]oxy}-N- methylacetamide |
| 320 | 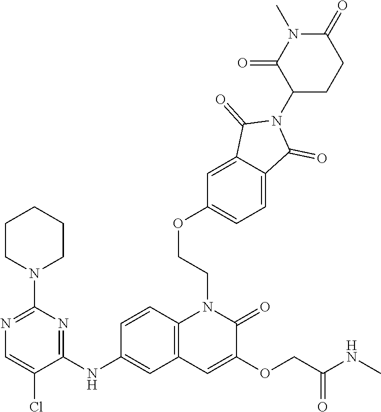 [SMILES: CNC(=O)COc1cc2cc(Nc3nc(N4CCCCC4)ncc3Cl)ccc2n(CCOc2ccc3c(c2)C(=O)N(C2CCC(=O)N(C)C2=O)C3=O)c1=O] | 2-[(6-{[5-chloro-2-(piperidin-1-yl)pyrimidin- 4-yl]amino}-1-(2-{[2-(1-methyl-2,6- dioxopiperidin-3-yl)-1,3-dioxo-2,3-dihydro- 1H-isoindol-5-yl]oxy}ethyl)-2-oxo-1,2- dihydroquinolin-3-yl)oxy]-N- methylacetamide |
| 321 | 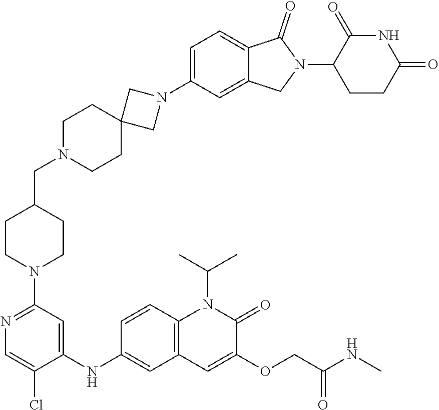 [SMILES: CNC(=O)COc1cc2cc(Nc3cc(N4CCC(CN5CCC6(CC5)CN(c5ccc7c(c5)CN(C5CCC(=O)NC5=O)C7=O)C6)CC4)ncc3Cl)ccc2n(C(C)C)c1=O] | 2-{[6-({5-chloro-2-[4-({2-[2-(2,6- dioxopiperidin-3-yl)-1-oxo-2,3-dihydro-1H- isoindol-5-yl]-2,7-diazaspiro[3.5]nonan-7- yl}methyl)piperidin-1-yl]pyridin-4- yl}amino)-2-oxo-1-(propan-2-yl)-1,2- dihydroquinolin-3-yl]oxy}-N- methylacetamide |
| 322 | 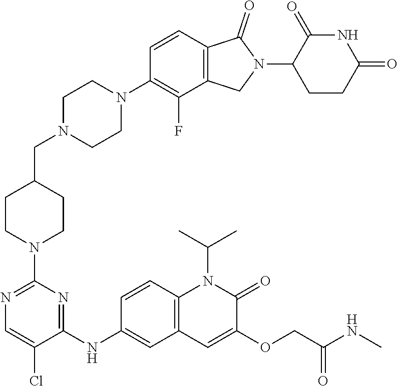 [SMILES: CNC(=O)COc1cc2cc(Nc3nc(N4CCC(CN5CCN(c6ccc7c(c6F)CN(C6CCC(=O)NC6=O)C7=O)CC5)CC4)ncc3Cl)ccc2n(C(C)C)c1=O] | 2-{[6-({5-chloro-2-[4-({4-[2-(2,6- dioxopiperidin-3-yl)-4-fluoro-1-oxo-2,3- dihydro-1H-isoindol-5-yl]piperazin-1- yl}methyl)piperidin-1-yl]pyrimidin-4- yl}amino)-2-oxo-1-(propan-2-yl)-1,2- dihydroquinolin-3-yl]oxy}-N- methylacetamide |
| 323 | 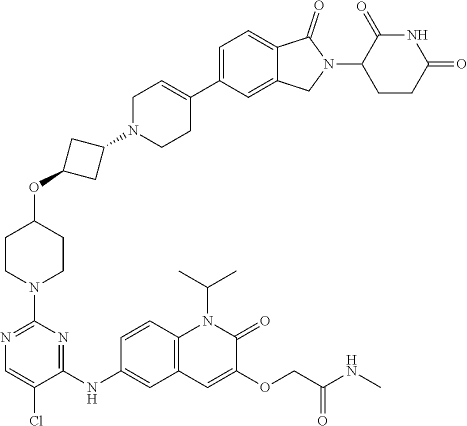 [SMILES: CNC(=O)COc1cc2cc(Nc3nc(N4CCC(O[C@H]5C[C@H](N6CC=C(c7ccc8c(c7)CN(C7CCC(=O)NC7=O)C8=O)CC6)C5)CC4)ncc3Cl)ccc2n(C(C)C)c1=O] | 2-({6-[(5-chloro-2-{4-[(1r,3r)-3-{4-[2-(2,6- dioxopiperidin-3-yl)-1-oxo-2,3-dihydro-1H- isoindol-5-yl]-1,2,3,6-tetrahydropyrridin-1- yl}cyclobutoxy]piperidin-1-yl}pyrimidin-4- yl)amino]-2-oxo-1-(propan-2-yl)-1,2- dihydroquinolin-3-yl}oxy)-N- methylacetamide |
| 324 | 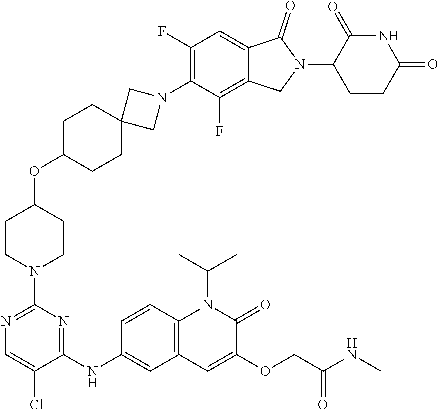 [SMILES: CNC(=O)COc1cc2cc(Nc3nc(N4CCC(OC5CCC6(CC5)CN(c5c(F)cc7c(c5F)CN(C5CCC(=O)NC5=O)C7=O)C6)CC4)ncc3Cl)ccc2n(C(C)C)c1=O] | 2-{[6-({5-chloro-2-[4-({2-[2-(2,6- dioxopiperidin-3-yl)-4,6-difluoro-1-oxo-2,3- dihydro-1H-isoindol-5-yl]-2- azaspiro[3.5]nonan-7-yl}oxy)piperidin-1- yl]pyrimidin-4-yl}amino)-2-oxo-1-(propan-2- yl)-1,2-dihydroquinolin-3-yl]oxy}-N- methylacetamide |
| 325 | 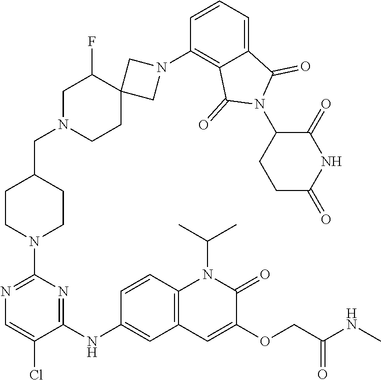 [SMILES: CNC(=O)COc1cc2cc(Nc3nc(N4CCC(CN5CCC6(CN(c7cccc8c7C(=O)N(C7CCC(=O)NC7=O)C8=O)C6)C(F)C5)CC4)ncc3Cl)ccc2n(C(C)C)c1=O] | 2-{[6-({5-chloro-2-[4-({2-[2-(2,6- dioxopiperidin-3-yl)-1,3-dioxo-2,3-dihydro- 1H-isoindol-4-yl]-5-fluoro-2,7- diazaspiro[3.5]nonan-7-yl}methyl)piperidin- 1-yl]pyrimidin-4-yl}amino)-2-oxo-1-(propan- 2-yl)-1,2-dihydroquinolin-3-yl]oxy}-N- methylacetamide |
| 326 | 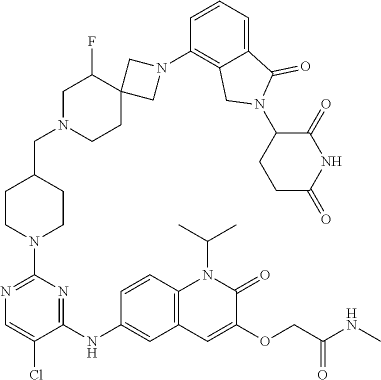 [SMILES: CNC(=O)COc1cc2cc(Nc3nc(N4CCC(CN5CCC6(CN(c7cccc8c7CN(C7CCC(=O)NC7=O)C8=O)C6)C(F)C5)CC4)ncc3Cl)ccc2n(C(C)C)c1=O] | 2-{[6-({5-chloro-2-[4-({2-[2-(2,6- dioxopiperidin-3-yl)-1-oxo-2,3-dihydro-1H- isoindol-4-yl]-5-fluoro-2,7- diazaspiro[3.5[nonan-7-yl}methyl)piperidin- 1-yl]pyrimidin-4-yl}amino)-2-oxo-1-(propan- 2-yl)-1,2-dihydroquinolin-3-yl]oxy}-N- methylacetamide |
| 327 | 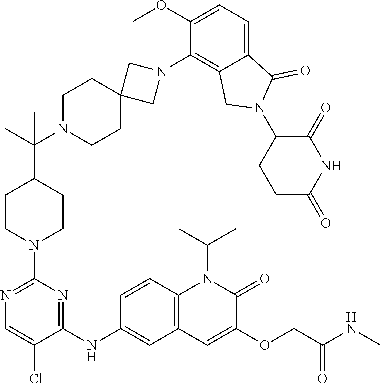 [SMILES: CNC(=O)COc1cc2cc(Nc3nc(N4CCC(C(C)(C)N5CCC6(CC5)CN(c5c(OC)ccc7c5CN(C5CCC(=O)NC5=O)C7=O)C6)CC4)ncc3Cl)ccc2n(C(C)C)c1=O] | 2-{[6-({5-chloro-2-[4-(2-{2-[2-(2,6- dioxopiperidin-3-yl)-5-methoxy-1-oxo-2,3- dihydro-1H-isoindol-4-yl]-2,7- diazaspiro[3.5]nonan-7-yl}propan-2- y)piperidin-1-yl]pyrimidin-4-yl}amino)-2- oxo-1-(propan-2-yl)-1,2-dihydroquinolin-3- yl]oxy}-N-methylacetamide |
| 328 | 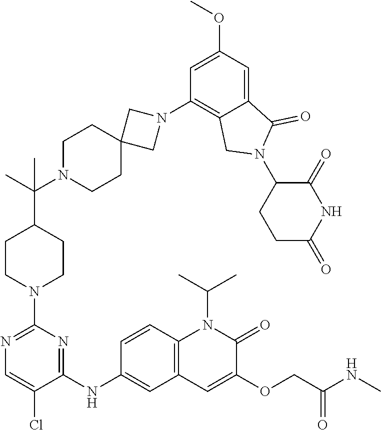 [SMILES: CNC(=O)COc1cc2cc(Nc3nc(N4CCC(C(C)(C)N5CCC6(CC5)CN(c5cc(OC)cc7c5CN(C5CCC(=O)NC5=O)C7=O)C6)CC4)ncc3Cl)ccc2n(C(C)C)c1=O] | 2-{[6-({5-chloro-2-[4-(2-{2-[2-(2,6- dioxopiperidin-3-yl)-6-methoxy-1-oxo-2,3- dihydro-1H-isoindol-4-yl]-2,7- diazaspiro[3.5]nonan-7-yl}propan-2- yl)piperidin-1-yl]pyrimidin-4-yl}amino)-2- oxo-1-(propan-2-yl)-1,2-dihydroquinolin-3- yl]oxy}-N-methylacetamide |
| 329 | 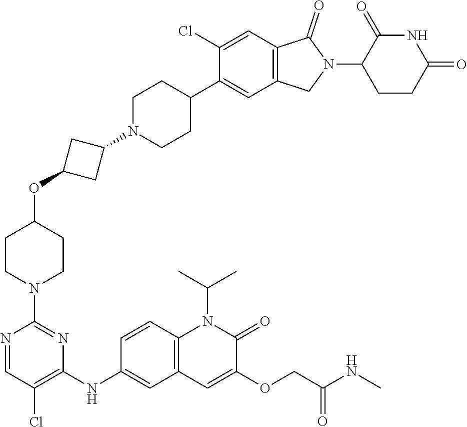 [SMILES: CNC(=O)COc1cc2cc(Nc3nc(N4CCC(O[C@H]5C[C@H](N6CCC(c7cc8c(cc7Cl)C(=O)N(C7CCC(=O)NC7=O)C8)CC6)C5)CC4)ncc3Cl)ccc2n(C(C)C)c1=O] | 2-({6-[(5-chloro-2-{4-[(1r,3r)-3-{4-[6-chloro- 2-(2,6-dioxopiperidin-3-yl)-1-oxo-2,3- dihydro-1H-isoindol-5-yl]piperidin-1- yl}cyclobutoxy]piperidin-1-yl}pyrimidin-4- yl)amino]-2-oxo-1-(propan-2-yl)-1,2- dihydroquinolin-3-yl}oxy)-N- methylacetamide |
| 330 | 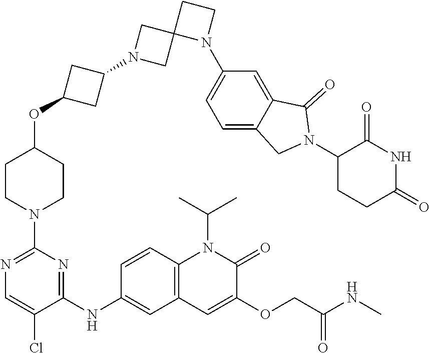 [SMILES: CNC(=O)COc1cc2cc(Nc3nc(N4CCC(O[C@H]5C[C@H](N6CC7(CCN7c7ccc8c(c7)C(=O)N(C7CCC(=O)NC7=O)C8)C6)C5)CC4)ncc3Cl)ccc2n(C(C)C)c1=O] | 2-({6-[(5-chloro-2-{4-[(1r,3r)-3-{1-[2-(2,6- dioxopiperidin-3-yl)-3-oxo-2,3-dihydro-1H- isoindol-5-yl]-1,6-diazaspiro[3.3]heptan-6- yl}cyclobutoxy]piperidin-1-yl}pyrimidin-4- yl)amino]-2-oxo-1-(propan-2-yl)-1,2- dihydroquinolin-3-yl}oxy)-N- methylacetamide |
| 331 | 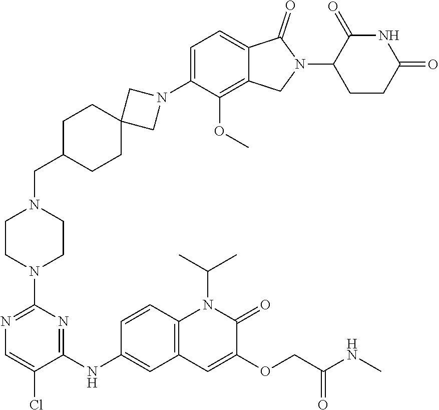 [SMILES: CNC(=O)COc1cc2cc(Nc3nc(N4CCN(CC5CCC6(CC5)CN(c5ccc7c(c5OC)CN(C5CCC(=O)NC5=O)C7=O)C6)CC4)ncc3Cl)ccc2n(C(C)C)c1=O] | 2-{[6-({5-chloro-2-[4-({2-[2-(2,6- dioxopiperidin-3-yl)-4-methoxy-1-oxo-2,3- dihydro-1H-isoindol-5-yl]-2- azaspiro[3.5]nonan-7-yl}methyl)piperazin-1- yl]pyrimidin-4-yl}amino)-2-oxo-1-(propan-2- yl)-1,2-dihydroquinolin-3-yl]oxy}-N- methylacetamide |
| 332 | 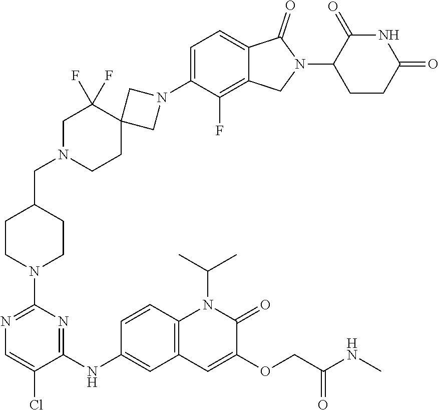 [SMILES: CNC(=O)COc1cc2cc(Nc3nc(N4CCC(CN5CCC6(CN(c7ccc8c(c7F)CN(C7CCC(=O)NC7=O)C8=O)C6)C(F)(F)C5)CC4)ncc3Cl)ccc2n(C(C)C)c1=O] | 2-{[6-({5-chloro-2-[4-({2-[2-(2,6- dioxopiperidin-3-yl)-4-fluoro-1-oxo-2,3- dihydro-1H-isoindol-5-yl]-5,5-difluoro-2,7- diazaspiro[3.5]nonan-7-yl}methyl)piperidin- 1-yl]pyrimidin-4-yl}amino)-2-oxo-1-(propan- 2-yl)-1,2-dihydroquinolin-3-yl]oxy}-N- methylacetamide |
| 333 | 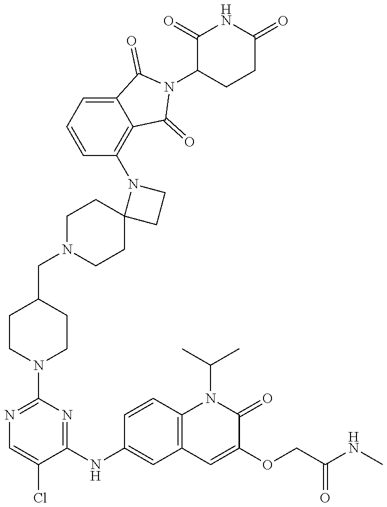 [SMILES: CNC(=O)COc1cc2cc(Nc3nc(N4CCC(CN5CCC6(CC5)CCN6c5cccc6c5C(=O)N(C5CCC(=O)NC5=O)C6=O)CC4)ncc3Cl)ccc2n(C(C)C)c1=O] | 2-{[6-({5-chloro-2-[4-({1-[2-(2,6- dioxopiperidin-3-yl)-1,3-dioxo-2,3-dihydro- 1H-isoindol-4-yl]-1,7-diazaspiro[3.5]nonan-7- yl}methyl)piperidin-1-yl]pyrimidin-4- yl}amino)-2-oxo-1-(propan-2-yl)-1,2- dihydroquinolin-3-yl]oxy}-N- methylacetamide |
| 334 | 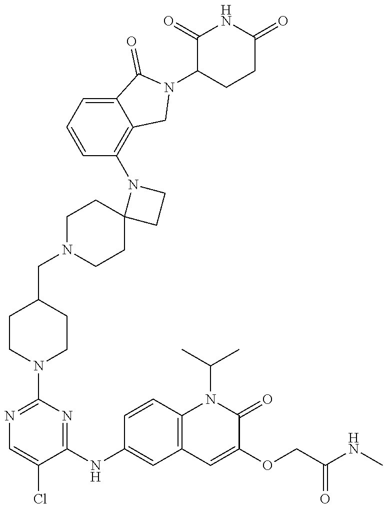 [SMILES: CNC(=O)COc1cc2cc(Nc3nc(N4CCC(CN5CCC6(CC5)CCN6c5cccc6c5CN(C5CCC(=O)NC5=O)C6=O)CC4)ncc3Cl)ccc2n(C(C)C)c1=O] | 2-{[6-({5-chloro-2-[4-({1-[2-(2,6- dioxopiperidin-3-yl)-1-oxo-2,3-dihydro-1H- isoindol-4-yl]-1,7-diazaspiro[3.5]nonan-7- yl}methyl)piperidin-1-yl]pyrimidin-4- yl}amino)-2-oxo-1-(propan-2-yl)-1,2- dihydroquinolin-3-yl]oxy}-N- methylacetamide |
| 335 | 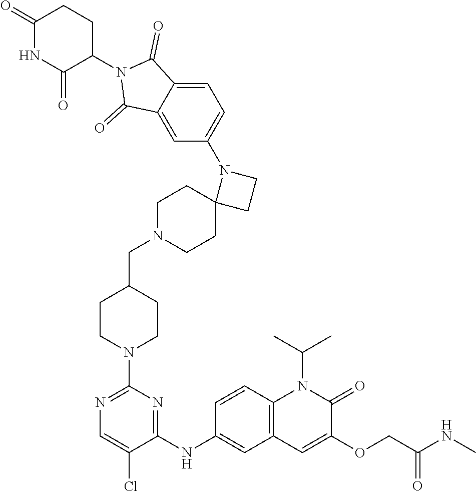 [SMILES: CNC(=O)COc1cc2cc(Nc3nc(N4CCC(CN5CCC6(CC5)CCN6c5ccc6c(c5)C(=O)N(C5CCC(=O)NC5=O)C6=O)CC4)ncc3Cl)ccc2n(C(C)C)c1=O] | 2-{[6-({5-chloro-2-[4-({1-[2-(2,6- dioxopiperidin-3-yl)-1,3-dioxo-2,3-dihydro- 1H-isoindol-5-yl]-1,7-diazaspiro[3.5]nonan-7- yl}methyl)piperidin-1-yl]pyrimidin-4- yl}amino)-2-oxo-1-(propan-2-yl)-1,2- dihydroquinolin-3-yl]oxy}-N- methylacetamide |
| 336 | 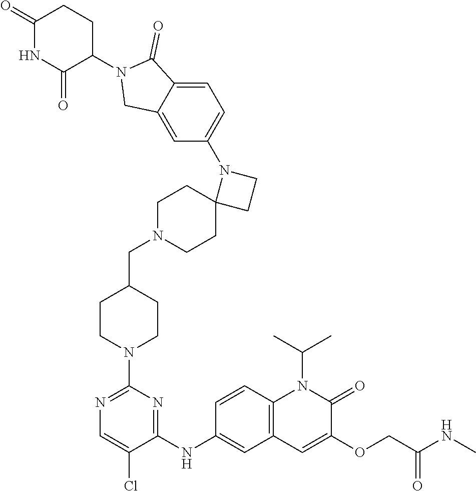 [SMILES: CNC(=O)COc1cc2cc(Nc3nc(N4CCC(CN5CCC6(CC5)CCN6c5ccc6c(c5)CN(C5CCC(=O)NC5=O)C6=O)CC4)ncc3Cl)ccc2n(C(C)C)c1=O] | 2-{[6-({5-chloro-2-[4-({1-[2-(2,6- dioxopiperidin-3-yl)-1-oxo-2,3-dihydro-1H- isoindol-5-yl]-1,7-diazaspiro[3.5]nonan-7- yl}methyl)piperidin-1-yl]pyrimidin-4- yl}amino)-2-oxo-1-(propan-2-yl)-1,2- dihydroquinolin-3-yl]oxy}-N- methylacetamide |
| 337 | 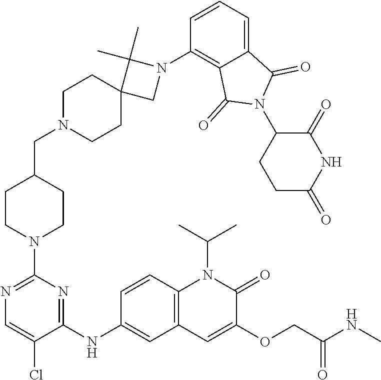 [SMILES: CNC(=O)COc1cc2cc(Nc3nc(N4CCC(CN5CCC6(CC5)CN(c5cccc7c5C(=O)N(C5CCC(=O)NC5=O)C7=O)C6(C)C)CC4)ncc3Cl)ccc2n(C(C)C)c1=O] | 2-{[6-({5-chloro-2-[4-({2-[2-(2,6- dioxopiperidin-3-yl)-1,3-dioxo-2,3-dihydro- 1H-isoindol-4-yl]-1,1-dimethyl-2,7- diazaspiro[3.5]nonan-7-yl}methyl)piperidin- 1-yl]pyrimidin-4-yl}amino)-2-oxo-1-(propan- 2-yl)-1,2-dihydroquinolin-3-yl]oxy}-N- methylacetamide |
| 338 | 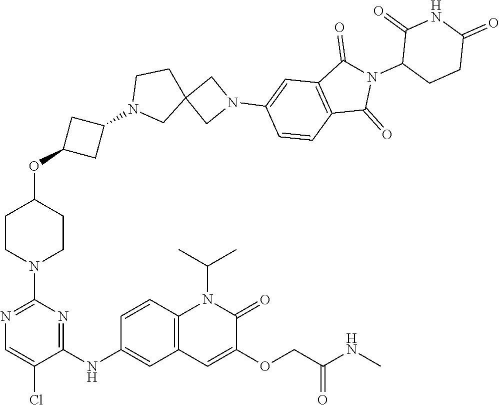 [SMILES: CNC(=O)COc1cc2cc(Nc3nc(N4CCC(O[C@H]5C[C@H](N6CCC7(CN(c8ccc9c(c8)C(=O)N(C8CCC(=O)NC8=O)C9=O)C7)C6)C5)CC4)ncc3Cl)ccc2n(C(C)C)c1=O] | 2-({6-[(5-chloro-2-{4-[(1r,3r)-3-{2-[2-(2,6- dioxopiperidin-3-yl)-1,3-dioxo-2,3-dihydro- 1H-isoindol-5-yl]-2,6-diazaspiro[3.4]octan-6- yl}cyclobutoxy]piperidin-1-yl}pyrimidin-4- yl)amino]-2-oxo-1-(propan-2-yl)-1,2- dihydroquinolin-3-yl}oxy)-N- methylacetamide |
| 339 | 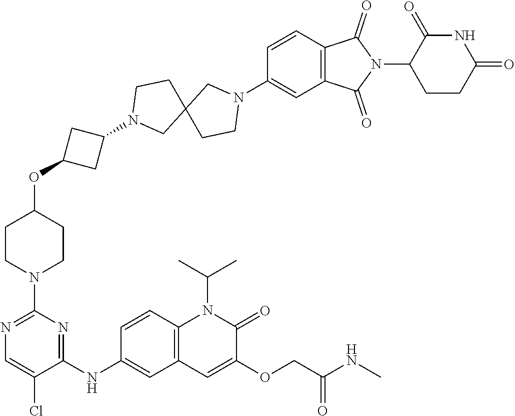 [SMILES: CNC(=O)COc1cc2cc(Nc3nc(N4CCC(O[C@H]5C[C@H](N6CCC7(CCN(c8ccc9c(c8)C(=O)N(C8CCC(=O)NC8=O)C9=O)C7)C6)C5)CC4)ncc3Cl)ccc2n(C(C)C)c1=O] | 2-({6-[(5-chloro-2-{4-[(1r,3r)-3-{7-[2-(2,6- dioxopiperidin-3-yl)-1,3-dioxo-2,3-dihydro- 1H-isoindol-5-yl]-2,7-diazaspiro[4.4]nonan-2- yl}cyclobutoxy]pieperidin-1-yl}pyrimidin-4- yl)amino]-2-oxo-1-(propan-2-yl)-1,2- dihydroquinolin-3-yl}oxy)-N- methylacetamide |
| 340 | 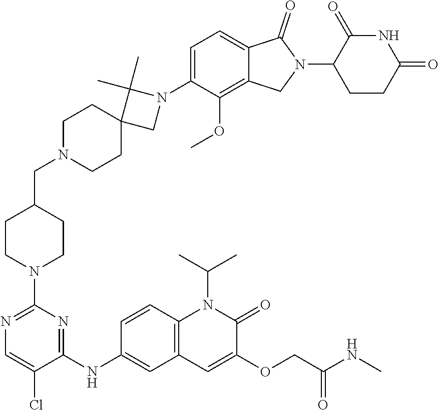 [SMILES: CNC(=O)COc1cc2cc(Nc3nc(N4CCC(CN5CCC6(CC5)CN(c5ccc7c(c5OC)CN(C5CCC(=O)NC5=O)C7=O)C6(C)C)CC4)ncc3Cl)ccc2n(C(C)C)c1=O] | 2-{[6-({5-chloro-2-[4-({2-[2-(2,6- dioxopiperidin-3-yl)-4-methoxy-1-oxo-2,3- dihydro-1H-isoindol-5-yl]-1,1-dimethyl-2,7- diazaspiro[3.5]nonan-7-yl}methyl)piperidin- 1-yl]pyrimidin-4-yl}amino)-2-oxo-1-(propan- 2-yl)-1,2-dihydroquinolin-3-yl]oxy}-N- methylacetamide |
| 341 | 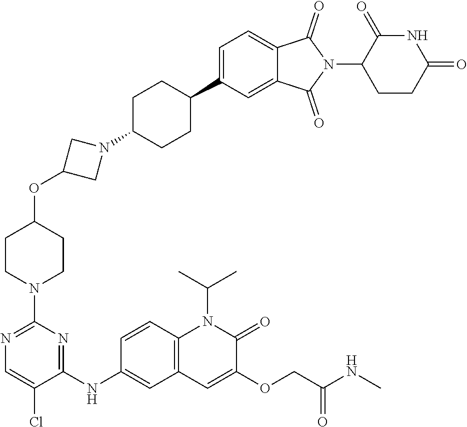 [SMILES: CNC(=O)COc1cc2cc(Nc3nc(N4CCC(OC5CN([C@H]6CC[C@H](c7ccc8c(c7)C(=O)N(C7CCC(=O)NC7=O)C8=O)CC6)C5)CC4)ncc3Cl)ccc2n(C(C)C)c1=O] | 2-{[6-({5-chloro-2-[4-({1-[(1r,4r)-4-[2-(2,6- dioxopiperidin-3-yl)-1,3-dioxo-2,3-dihydro- 1H-isoindol-5-yl]cyclohexyl]azetidin-3- yl}oxy)piperidin-1-yl]pyrimidin-4-yl}amino)- 2-oxo-1-(propan-2-yl)-1,2-dihydroquinolin-3- yl]oxy}-N-methylacetamide |
| 342 | 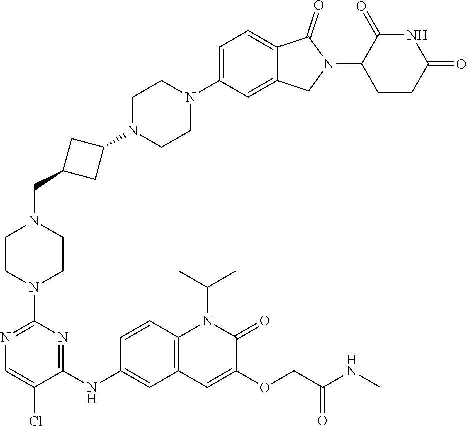 [SMILES: CNC(=O)COc1cc2cc(Nc3nc(N4CCN(C[C@H]5C[C@H](N6CCN(c7ccc8c(c7)CN(C7CCC(=O)NC7=O)C8=O)CC6)C5)CC4)ncc3Cl)ccc2n(C(C)C)c1=O] | 2-[(6-{[5-chloro-2-(4-{[(1r,3r)-3-{4-[2-(2,6- dioxopiperidin-3-yl)-1-oxo-2,3-dihydro-1H- isoindol-5-yl]piperazin-1- yl}cyclobutyl]methyl}piperazin-1- yl)pyrimidin-4-yl]amino}-2-oxo-1-(propan-2- yl)-1,2-dihydroquinolin-3-yl)oxy]-N- methylacetamide |
| 343 | 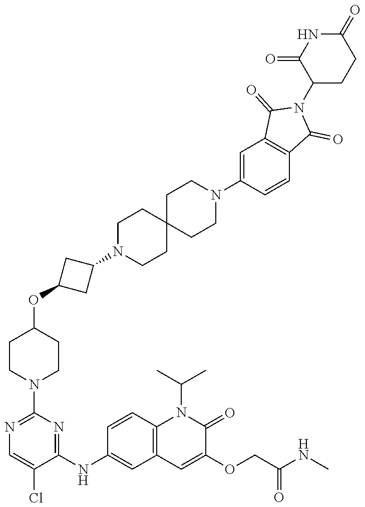 [SMILES: CNC(=O)COc1cc2cc(Nc3nc(N4CCC(O[C@H]5C[C@H](N6CCC7(CCN(c8ccc9c(c8)C(=O)N(C8CCC(=O)NC8=O)C9=O)CC7)CC6)C5)CC4)ncc3Cl)ccc2n(C(C)C)c1=O] | 2-({6-[(5-chloro-2-{4-[(1r,3r)-3-{9-[2-(2,6- dioxopiperidin-3-yl)-1,3-dioxo-2,3-dihydro- 1H-isoindol-5-yl]-3,9- diazaspiro[5.5]undecan-3- yl}cyclobutoxy]piperidin-1-yl}pyrimidin-4- yl)amino]-2-oxo-1-(propan-2-yl)-1,2- dihydroquinolin-3-yl}oxy)-N- methylacetamide |
| 344 | 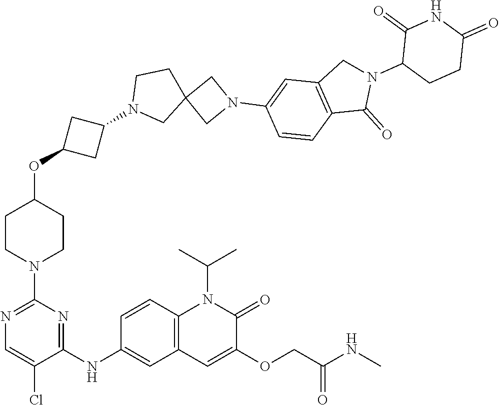 [SMILES: CNC(=O)COc1cc2cc(Nc3nc(N4CCC(O[C@H]5C[C@H](N6CCC7(CN(c8ccc9c(c8)CN(C8CCC(=O)NC8=O)C9=O)C7)C6)C5)CC4)ncc3Cl)ccc2n(C(C)C)c1=O] | 2-({6-[(5-chloro-2-{4-[(1r,3r)-3-{2-[2-(2,6- dioxopiperidin-3-yl)-1-oxo-2,3-dihydro-1H- isoindol-5-yl]-2,6-diazaspiro[3.4]octan-6- yl}cyclobutoxy]piperidni-1-yl}pyrimidin-4- yl)amino]-2-oxo-1-(propan-2-yl)-1,2- dihydroquinolin-3-yl}oxy)-N- methylacetamide |
| 345 | 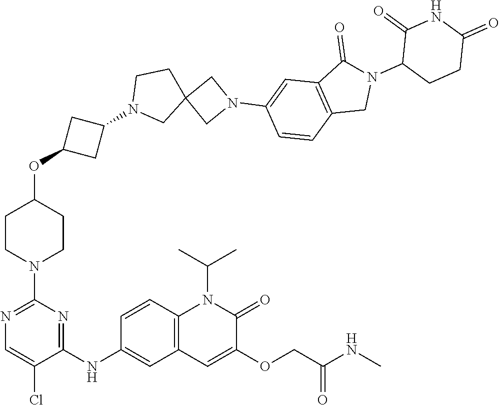 [SMILES: CNC(=O)COc1cc2cc(Nc3nc(N4CCC(O[C@H]5C[C@H](N6CCC7(CN(c8ccc9c(c8)C(=O)N(C8CCC(=O)NC8=O)C9)C7)C6)C5)CC4)ncc3Cl)ccc2n(C(C)C)c1=O] | 2-({6-[(5-chloro-2-{4-[(1r,3r)-3-{2-[2-(2,6- dioxopiperidin-3-yl)-3-oxo-2,3-dihydro-1H- isoindol-5-yl]-2,6-diazaspiro[3.4]octan-6- yl}cyclobutoxy]piperidin-1-yl}pyrimidin-4- yl)amino]-2-oxo-1-(propan-2-yl)-1,2- dihydroquinolin-3-yl}oxy)-N- methylacetamide |
| 346 | 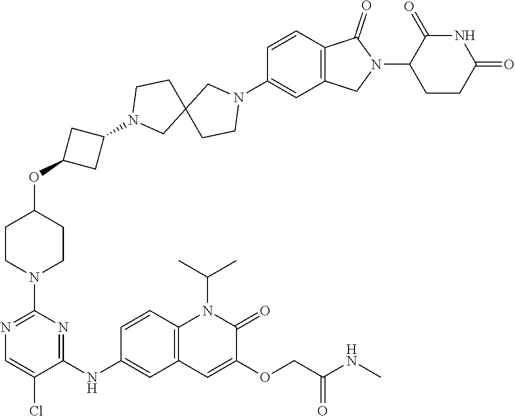 [SMILES: CNC(=O)COc1cc2cc(Nc3nc(N4CCC(O[C@H]5C[C@H](N6CCC7(CCN(c8ccc9c(c8)CN(C8CCC(=O)NC8=O)C9=O)C7)C6)C5)CC4)ncc3Cl)ccc2n(C(C)C)c1=O] | 2-({6-[(5-chloro-2-{4-[(1r,3r)-3-{7-[2-(2,6- dioxopiperidin-3-yl)-1-oxo-2,3-dihydro-1H- isoindol-5-yl]-2,7-diazaspiro[4.4]nonan-2- yl}cyclobutoxy]piperidin-1-yl}pyrimdiin-4- yl)amino]-2-oxo-1-(propan-2-yl)-1,2- dihydroquinolin-3-yl}oxy)-N- methylacetamide |
| 347 | 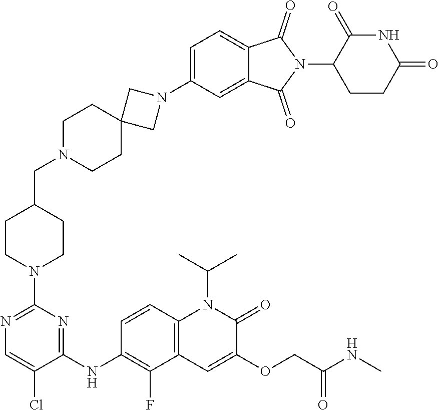 [SMILES: CNC(=O)COc1cc2c(F)c(Nc3nc(N4CCC(CN5CCC6(CC5)CN(c5ccc7c(c5)C(=O)N(C5CCC(=O)NC5=O)C7=O)C6)CC4)ncc3Cl)ccc2n(C(C)C)c1=O] | 2-{[6-({5-chloro-2-[4-({2-[2-(2,6- dioxopiperidin-3-yl)-1,3-dioxo-2,3-dihydro- 1H-isoindol-5-yl]-2,7-diazaspiro[3.5]nonan-7- yl}methyl)piperidin-1-yl]pyrimidin-4- yl}amino)-5-fluoro-2-oxo-1-(propan-2-yl)- 1,2-dihydroquinolin-3-yl]oxy}-N- methylacetamide |
| 348 | 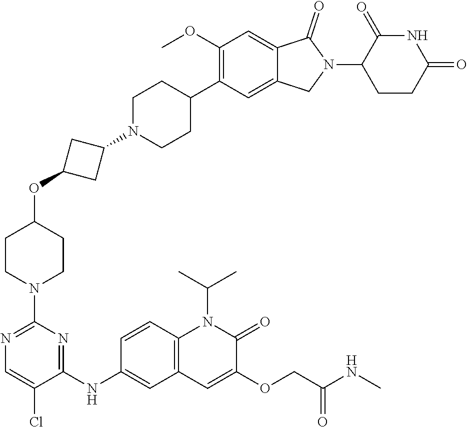 [SMILES: CNC(=O)COc1cc2cc(Nc3nc(N4CCC(O[C@H]5C[C@H](N6CCC(c7cc8c(cc7OC)C(=O)N(C7CCC(=O)NC7=O)C8)CC6)C5)CC4)ncc3Cl)ccc2n(C(C)C)c1=O] | 2-({6-[(5-chloro-2-{4-[(1r,3r)-3-{4-[2-(,26- dioxopiperidin-3-yl)-6-methoxy-1-oxo-2,3- dihydro-1H-isoindol-5-yl]piperidin-1- yl}cyclobutoxy]piperidin-1-yl}pyrimdiin-4- yl)amino]-2-oxo-1-(propan-2-yl)-1,2- dihydroquinolin-3-yl}oxy)-N- methylacetamide |
| 349 | 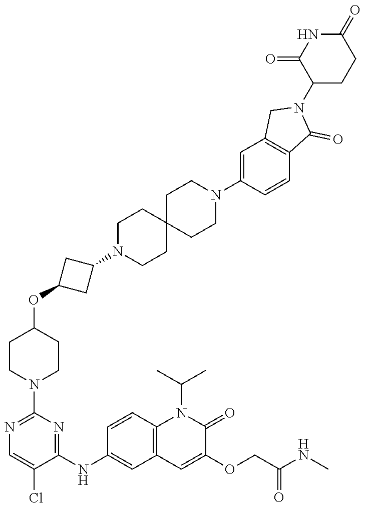 [SMILES: CNC(=O)COc1cc2cc(Nc3nc(N4CCC(O[C@H]5C[C@H](N6CCC7(CCN(c8ccc9c(c8)CN(C8CCC(=O)NC8=O)C9=O)CC7)CC6)C5)CC4)ncc3Cl)ccc2n(C(C)C)c1=O] | 2-({6-[(5-chloro-2-{4-[(1r,3r)-3-{9-[2-(2,6- dioxopiperidin-3-yl)-1-oxo-2,3-dihydro-1H- isoindol-5-yl]-3,9-diazaspiro[5.5]undecna-3- yl}cyclobutoxy]piperidin-1-yl}pyrimidin-4- yl)amino]-2-oxo-1-(propan-2-yl)-1,2- dihydroquinolin-3-yl}oxy)-N- methylacetamide |
| 350 | 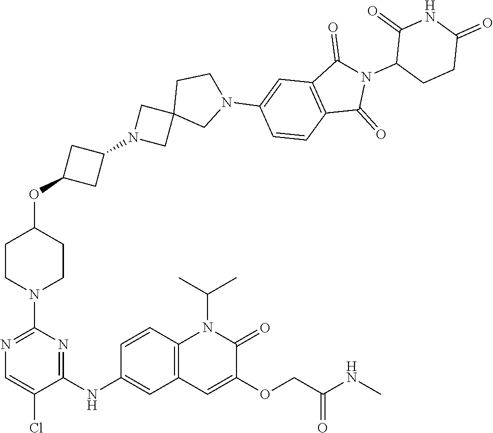 [SMILES: CNC(=O)COc1cc2cc(Nc3nc(N4CCC(O[C@H]5C[C@H](N6CC7(CCN(c8ccc9c(c8)C(=O)N(C8CCC(=O)NC8=O)C9=O)C7)C6)C5)CC4)ncc3Cl)ccc2n(C(C)C)c1=O] | 2-({6-[(5-chloro-2-{4-[(1r,3r)-3-{6-[2-(2,6- dioxopiperidin-3-yl)-1,3-dioxo-2,3-dihydro- 1H-isoindol-5-yl]-2,6-diazaspiro[3.4]octan-2- yl}cyclobutoxy]piperidin-1-yl}pyrimidin-4- yl)amino]-2-oxo-1-(propan-2-yl)-1,2- dihydroquinolin-3-yl}oxy)-N- methylacetamide |
| 351 | 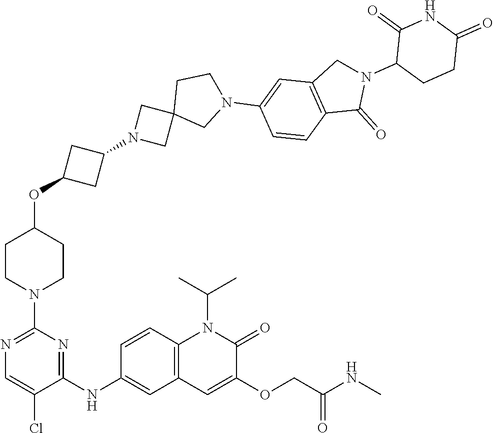 [SMILES: CNC(=O)COc1cc2cc(Nc3nc(N4CCC(O[C@H]5C[C@H](N6CC7(CCN(c8ccc9c(c8)CN(C8CCC(=O)NC8=O)C9=O)C7)C6)C5)CC4)ncc3Cl)ccc2n(C(C)C)c1=O] | 2-({6-[(5-chloro-2-{4-[(1r,3r)-3-{6-[2-(2,6- dioxopiperidin-3-yl)-1-oxo-2,3-dihydro-1H- isoindol-5-yl]-2,6-diazaspiro[3.4]octan-2- yl}cyclobutoxy]piperidin-1-yl}pyrimidin-4- yl)amino]-2-oxo-1-(propan-2-yl)-1,2- dihydroquinolin-3-yl}oxy)-N- methylacetamide |
| 352 | 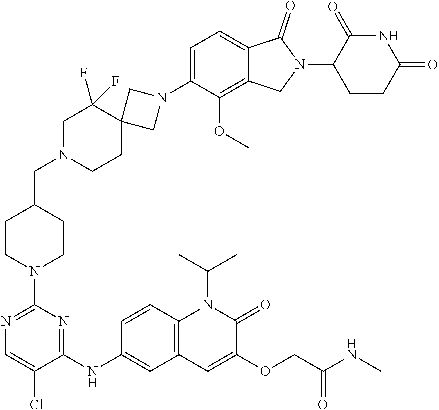 [SMILES: CNC(=O)COc1cc2cc(Nc3nc(N4CCC(CN5CCC6(CN(c7ccc8c(c7OC)CN(C7CCC(=O)NC7=O)C8=O)C6)C(F)(F)C5)CC4)ncc3Cl)ccc2n(C(C)C)c1=O] | 2-{[6-({5-chloro-2-[4-({2-[2-(2,6- dioxopiperidin-3-yl)-4-methoxy-1-oxo-2,3- dihydro-1H-isoindol-5-yl]-5,5-difluoro-2,7- diazaspiro[3.5]nonan-7-yl}methyl)piperidin- 1-yl]pyrimidin-4-yl}amino)-2-oxo-1-(propan- 2-yl)-1,2-dihydroquinolin-3-yl]oxy}-N- methylacetamide |
| 353 | 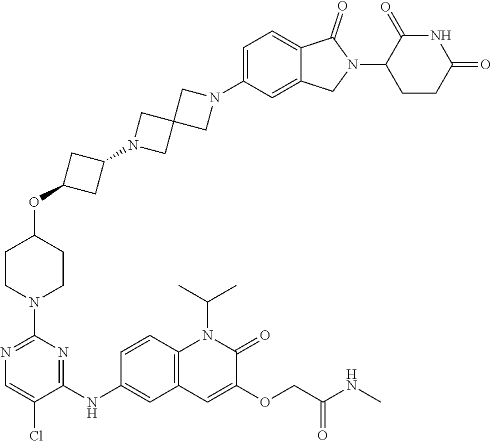 [SMILES: CNC(=O)COc1cc2cc(Nc3nc(N4CCC(O[C@H]5C[C@H](N6CC7(CN(c8ccc9c(c8)CN(C8CCC(=O)NC8=O)C9=O)C7)C6)C5)CC4)ncc3Cl)ccc2n(C(C)C)c1=O] | 2-({6-[(5-chloro-2-{4-[(1r,3r)-3-{6-[2-(2,6- dioxopiperidin-3-yl)-1-oxo-2,3-dihydro-1H- isoindol-5-yl]-2,6-diazaspiro[3.3]heptan-2- yl}cyclobutoxy]piperidin-1-yl}pyrimidin-4- yl)amino]-2-oxo-1-(propan-2-yl)-1,2- dihydroquinolin-3-yl}oxy)-N- methylacetamide |
| 354 | 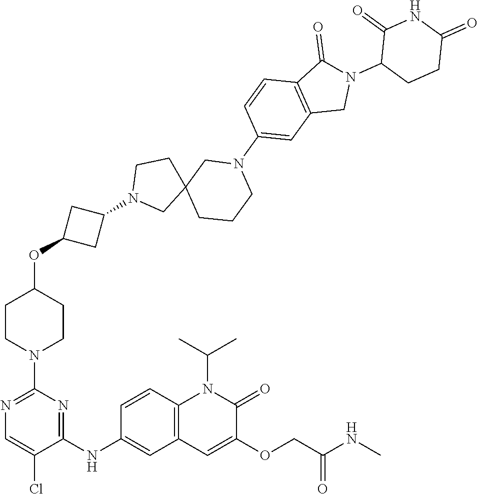 [SMILES: CNC(=O)COc1cc2cc(Nc3nc(N4CCC(O[C@H]5C[C@H](N6CCC7(CCCN(c8ccc9c(c8)CN(C8CCC(=O)NC8=O)C9=O)C7)C6)C5)CC4)ncc3Cl)ccc2n(C(C)C)c1=O] | 2-({6-[(5-chloro-2-{4-[(1r,3r)-3-{7-[2-(2,6- dioxopiperidin-3-yl)-1-oxo-2,3-dihydro-1H- isoindol-5-yl]-2,7-diazaspiro[4.5]decan-2- yl}cyclobutoxy]piperidin-1-yl}pyrimidin-4- yl)amino]-2-oxo-1-(propan-2-yl)-1,2- dihydroquinolin-3-yl}oxy)-N- methylacetamide |
| 355 | 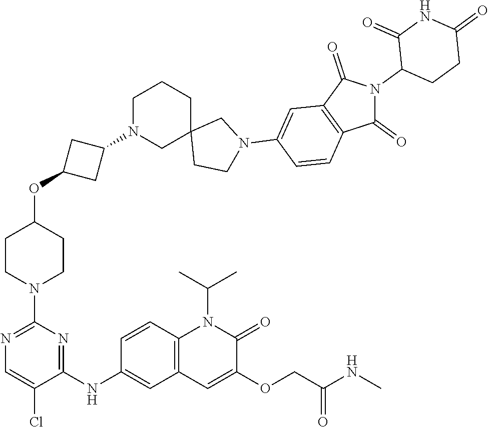 [SMILES: CNC(=O)COc1cc2cc(Nc3nc(N4CCC(O[C@H]5C[C@H](N6CCCC7(CCN(c8ccc9c(c8)C(=O)N(C8CCC(=O)NC8=O)C9=O)C7)C6)C5)CC4)ncc3Cl)ccc2n(C(C)C)c1=O] | 2-({6-[(5-chloro-2-{4-[(1r,3r)-3-{2-[2-(2,6- dioxopiperidin-3-yl)-1,3-dioxo-2,3-dihydro- 1H-isoindol-5-yl]-2,7-diazaspiro[4.5]decan-7- yl}cyclobutoxy]piperidin-1-yl}pyrimidin-4- yl)amino]-2-oxo-1-(propan-2-yl)-1,2- dihydroquinolin-3-yl}oxy)-N- methylacetamide |
| 356 | 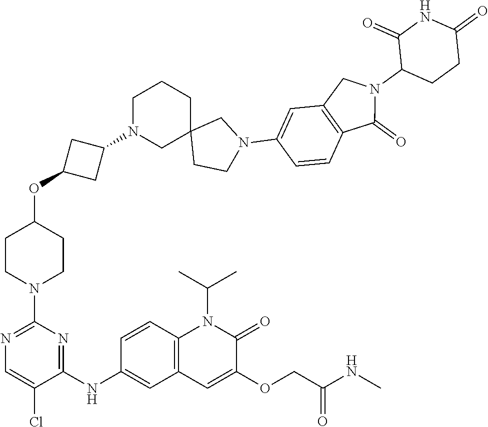 [SMILES: CNC(=O)COc1cc2cc(Nc3nc(N4CCC(O[C@H]5C[C@H](N6CCCC7(CCN(c8ccc9c(c8)CN(C8CCC(=O)NC8=O)C9=O)C7)C6)C5)CC4)ncc3Cl)ccc2n(C(C)C)c1=O] | 2-({6-[(5-chloro-2-{4-[(1r,3r)-3-{2-[2-(2,6- dioxopiperidin-3-yl)-1-oxo-2,3-dihydro-1H- isoindol-5-yl]-2,7-diazaspiro[4.5]decan-7- yl}cyclobutoxy]piperidin-1-yl}pyrimidin-4- yl)amino]-2-oxo-1-(propan-2-yl)-1,2- dihydroquinolin-3-yl}oxy)-N- methylacetamide |
| 357 | 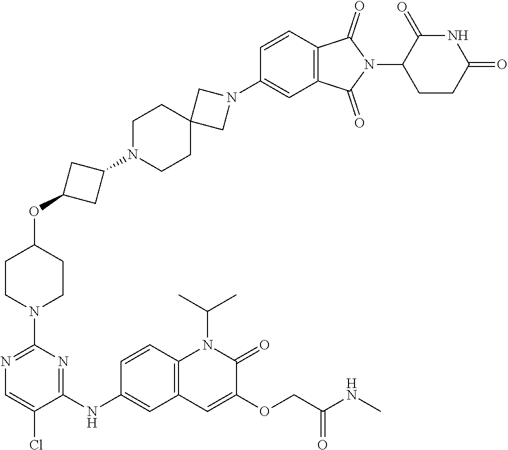 [SMILES: CNC(=O)COc1cc2cc(Nc3nc(N4CCC(O[C@H]5C[C@H](N6CCC7(CC6)CN(c6ccc8c(c6)C(=O)N(C6CCC(=O)NC6=O)C8=O)C7)C5)CC4)ncc3Cl)ccc2n(C(C)C)c1=O] | 2-({6-[(5-chloro-2-{4-[(1r,3r)-3-{2-[2-(2,6- dioxopiperidin-3-yl)-1,3-dioxo-2,3-dihydro- 1H-isoindol-5-yl]-2,7-diazaspiro[3.5]nonan-7- yl}cyclobutoxy]piperidin-1-yl}pyrimidin-4- yl)amino]-2-oxo-1-(propan-2-yl)-1,2- dihydroquinolin-3-yl}oxy)-N- methylacetamide |
| 358 | 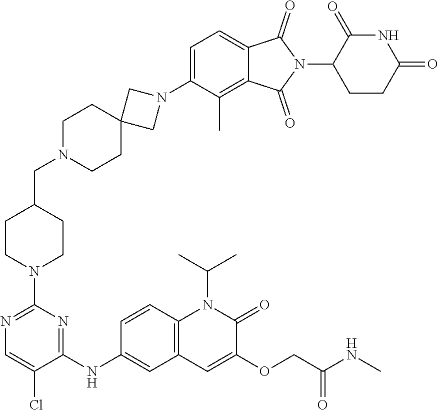 [SMILES: CNC(=O)COc1cc2cc(Nc3nc(N4CCC(CN5CCC6(CC5)CN(c5ccc7c(c5C)C(=O)N(C5CCC(=O)NC5=O)C7=O)C6)CC4)ncc3Cl)ccc2n(C(C)C)c1=O] | 2-{[6-({5-chloro-2-[4-({2-[2-(2,6- dioxopiperidin-3-yl)-4-methyl-1,3-dioxo-2,3- dihydro-1H-isoindol-5-yl]-2,7- diazaspiro[3.5]nonan-7-yl}methyl)piperidin- 1-yl]pyrimidin-4-yl}amino)-2-oxo-1-(propan- 2-yl)-1,2-dihydroquinolin-3-yl]oxy}-N- methylacetamide |
| 359 | 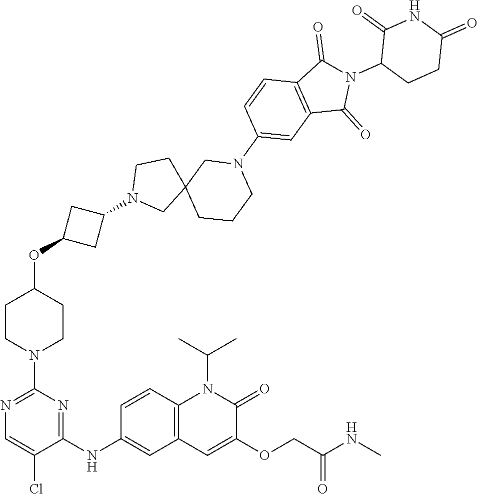 [SMILES: CNC(=O)COc1cc2cc(Nc3nc(N4CCC(O[C@H]5C[C@H](N6CCC7(CCCN(c8ccc9c(c8)C(=O)N(C8CCC(=O)NC8=O)C9=O)C7)C6)C5)CC4)ncc3Cl)ccc2n(C(C)C)c1=O] | 2-({6-[(5-chloro-2-{4-[(1r,3r)-3-{7-[2-(2,6- dioxopiperidin-3-yl)-1,3-dioxo-2,3-dihydro- 1H-isoindol-5-yl]-2,7-diazaspiro[4.5]decan-2- yl}cyclobutoxy]piperidin-1-yl}pyrimidin-4- yl)amino]-2-oxo-1-(propan-2-yl)-1,2- dihydroquinolin-3-yl}oxy)-N- methylacetamide |
| 360 | 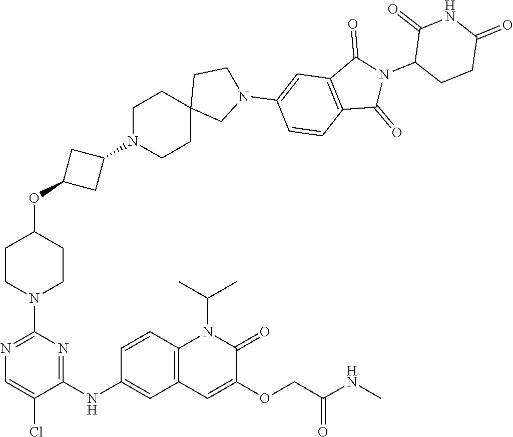 [SMILES: CNC(=O)COc1cc2cc(Nc3nc(N4CCC(O[C@H]5C[C@H](N6CCC7(CCN(c8ccc9c(c8)C(=O)N(C8CCC(=O)NC8=O)C9=O)C7)CC6)C5)CC4)ncc3Cl)ccc2n(C(C)C)c1=O] | 2-({6-[(5-chloro-2-{4-[(1r,3r)-3-{2-[2-(2,6- dioxopiperidin-3-yl)-1,3-dioxo-2,3-dihydro- 1H-isoindol-5-yl]-2,8-diazaspiro[4.5]decan-8- yl}cyclobutoxy]piperidin-1-yl}pyrimidin-4- yl)amino]-2-oxo-1-(propan-2-yl)-1,2- dihydroquinolin-3-yl}oxy)-N- methylacetamide |
| 361 | 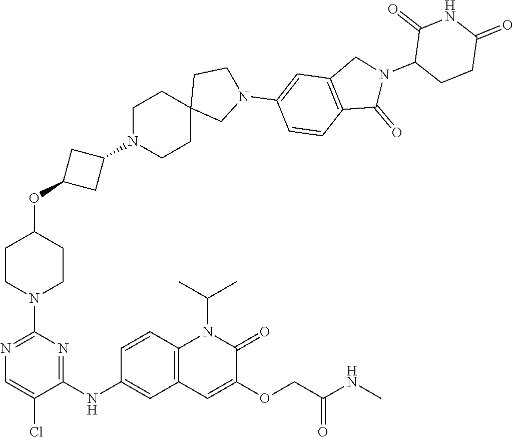 [SMILES: CNC(=O)COc1cc2cc(Nc3nc(N4CCC(O[C@H]5C[C@H](N6CCC7(CCN(c8ccc9c(c8)CN(C8CCC(=O)NC8=O)C9=O)C7)CC6)C5)CC4)ncc3Cl)ccc2n(C(C)C)c1=O] | 2-({6-[(5-chloro-2-{4-[(1r,3r)-3-{2-[2-(2,6- dioxopiperidin-3-yl)-1-oxo-2,3-dihydro-1H- isoindol-5-yl]-2,8-diazaspiro[4.5]decan-8- yl}cyclobutoxy]piperidin-1-yl}pyrimidin-4- yl)amino]-2-oxo-1-(propan-2-yl)-1,2- dihydroquinolin-3-yl}oxy)-N- methylacetamide |
| 362 | 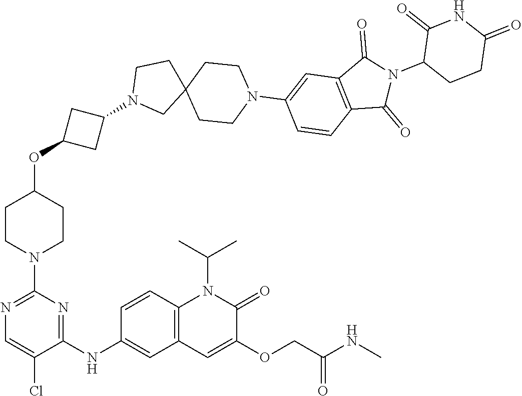 [SMILES: CNC(=O)COc1cc2cc(Nc3nc(N4CCC(O[C@H]5C[C@H](N6CCC7(CCN(c8ccc9c(c8)C(=O)N(C8CCC(=O)NC8=O)C9=O)CC7)C6)C5)CC4)ncc3Cl)ccc2n(C(C)C)c1=O] | 2-({6-[(5-chloro-2-{4-[(1r,3r)-3-{8-[2-(2,6- dioxopiperidin-3-yl)-1,3-dioxo-2,3-dihydro- 1H-isoindol-5-yl]-2,8-diazaspiro[4.5]decan-2- yl}cyclobutoxy]piperidin-1-yl}pyrimidin-4- yl)amino]-2-oxo-1-(propan-2-yl)-1,2- dihydroquinolin-3-yl}oxy)-N- methylacetamide |
| 363 | 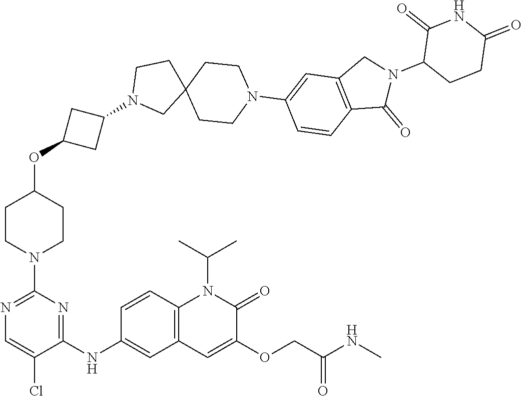 [SMILES: CNC(=O)COc1cc2cc(Nc3nc(N4CCC(O[C@H]5C[C@H](N6CCC7(CCN(c8ccc9c(c8)CN(C8CCC(=O)NC8=O)C9=O)CC7)C6)C5)CC4)ncc3Cl)ccc2n(C(C)C)c1=O] | 2-({6-[(5-chloro-2-{4-[(1r,3r)-3-{8-[2-(2,6- dioxopiperidin-3-yl)-1-oxo-2,3-dihydro-1H- isoindol-5-yl]-2,8-diazaspiro[4.5]decan-2- yl}cyclobutoxy]piperidin-1-yl}pyrimidin-4- yl)amino]-2-oxo-1-(propan-2-yl)-1,2- dihydroquinolin-3-yl}oxy)-N- methylacetamide |
| 364 | 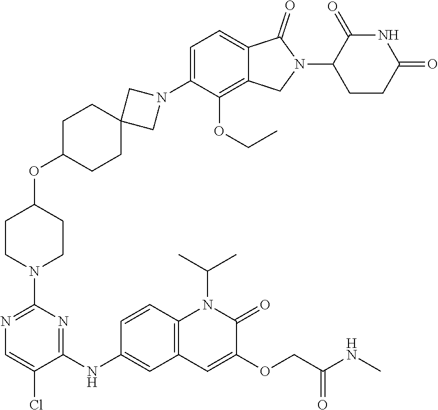 [SMILES: CCOc1c(N2CC3(CCC(OC4CCN(c5ncc(Cl)c(Nc6ccc7c(c6)cc(OCC(=O)NC)c(=O)n7C(C)C)n5)CC4)CC3)C2)ccc2c1CN(C1CCC(=O)NC1=O)C2=O] | 2-{[6-({5-chloro-2-[4-({2-[2-(2,6- dioxopiperidin-3-yl)-4-ethoxy-1-oxo-2,3- dihydro-1H-isoindol-5-yl]-2- azaspiro[3.5]nonan-7-yl}oxy)piperidin-1- yl]pyrimidin-4-yl}amino)-2-oxo-1-(proapn-2- yl)-1,2-dihydroquinolin-3-yl]oxy}-N- methylacetamide |
| 365 | 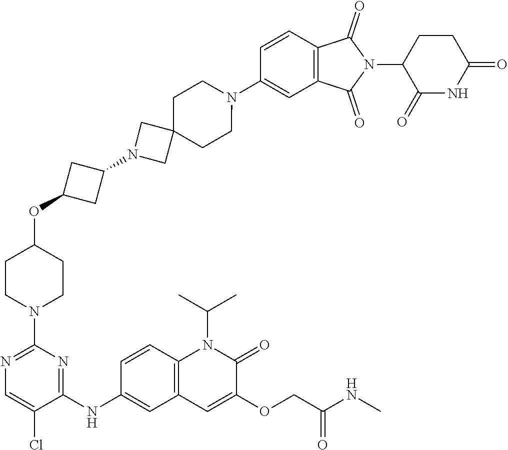 [SMILES: CNC(=O)COc1cc2cc(Nc3nc(N4CCC(O[C@H]5C[C@H](N6CC7(CCN(c8ccc9c(c8)C(=O)N(C8CCC(=O)NC8=O)C9=O)CC7)C6)C5)CC4)ncc3Cl)ccc2n(C(C)C)c1=O] | 2-({6-[(5-chloro-2-{4-[(1r,3r)-3-{7-[2-(2,6- dioxopiperidin-3-yl)-1,3-dioxo-2,3-dihydro- 1H-isoindol-5-yl]-2,7-diazaspiro[3.5]nonan-2- yl}cyclobutoxy]piperidin-1-yl}pyrimidin-4- yl)amino]-2-oxo-1-(propan-2-yl)-1,2- dihydroquinolin-3-yl}oxy)-N- methylacetamide |
| 366 | 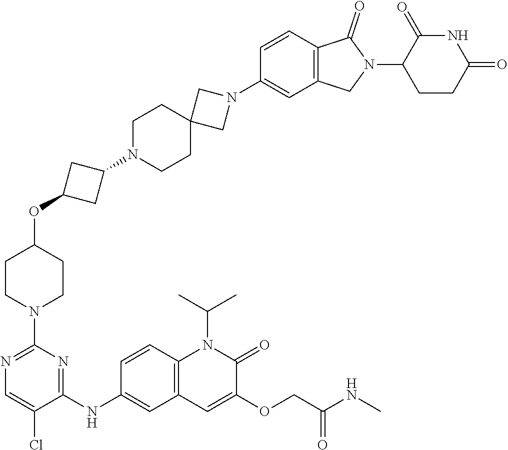 [SMILES: CNC(=O)COc1cc2cc(Nc3nc(N4CCC(O[C@H]5C[C@H](N6CCC7(CC6)CN(c6ccc8c(c6)CN(C6CCC(=O)NC6=O)C8=O)C7)C5)CC4)ncc3Cl)ccc2n(C(C)C)c1=O] | 2-({6-[(5-chloro-2-{4-[(1r,3r)-3-{2-[2-(2,6- dioxopiperidin-3-yl)-1-oxo-2,3-dihydro-1H- isoindol-5-yl]-2,7-diazaspiro[3.5]nonan-7- yl}cyclobutoxy]piperidin-1-yl}pyrimidin-4- yl)amino]-2-oxo-1-(propan-2-yl)-1,2- dihydroquinolin-3-yl}oxy)-N- methylacetamide |
| 367 | 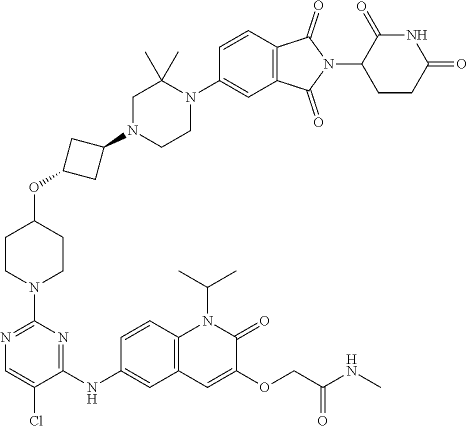 [SMILES: CNC(=O)COc1cc2cc(Nc3nc(N4CCC(O[C@H]5C[C@H](N6CCN(c7ccc8c(c7)C(=O)N(C7CCC(=O)NC7=O)C8=O)C(C)(C)C6)C5)CC4)ncc3Cl)ccc2n(C(C)C)c1=O] | 2-({6-[(5-chloro-2-{4-[(1r,3r)-3-{4-[2-(2,6- dioxopiperidin-3-yl)-1,3-dioxo-2,3-dihydro- 1H-isoindol-5-yl]-3,3-dimethylpiperazin-1- yl}cyclobutoxy]piperidin-1-yl}pyrimidin-4- yl)amino]-2-oxo-1-(propan-2-yl)-1,2- dihydroquinolin-3-yl}oxy)-N- methylacetamide |
| 368 | 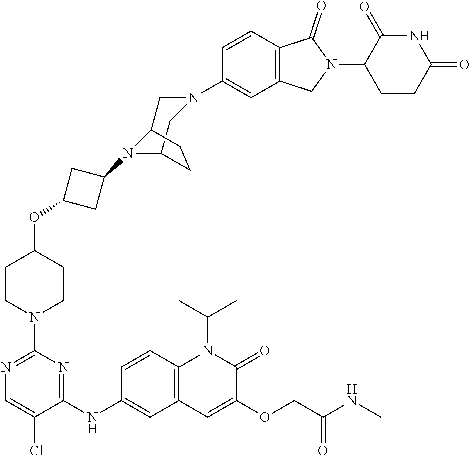 [SMILES: CNC(=O)COc1cc2cc(Nc3nc(N4CCC(O[C@H]5C[C@H](N6C7CCC6CN(c6ccc8c(c6)CN(C6CCC(=O)NC6=O)C8=O)C7)C5)CC4)ncc3Cl)ccc2n(C(C)C)c1=O] | 2-({6-[(5-chloro-2-{4-[(1r,3r)-3-{3-[2-(2,6- dioxopiperidin-3-yl)-1-oxo-2,3-dihydro-1H- isoindol-5-yl]-3,8-diazabicyclo[3.2.1]octan-8- yl}cyclobutoxy]piperidin-1-yl}pyrimidin-4- yl)amino]-2-oxo-1-(propan-2-yl)-1,2- dihydroquinolin-3-yl}oxy)-N- methylacetamide |
| 369 | 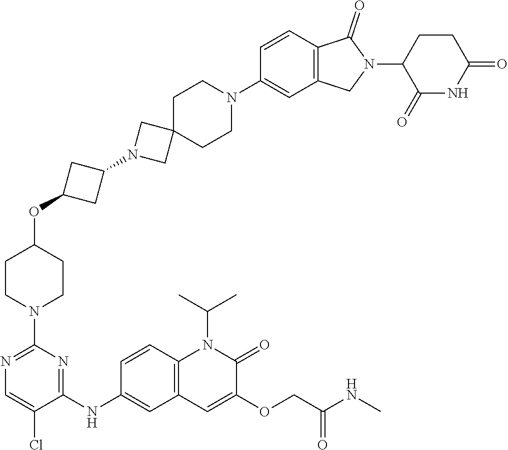 [SMILES: CNC(=O)COc1cc2cc(Nc3nc(N4CCC(O[C@H]5C[C@H](N6CC7(CCN(c8ccc9c(c8)CN(C8CCC(=O)NC8=O)C9=O)CC7)C6)C5)CC4)ncc3Cl)ccc2n(C(C)C)c1=O] | 2-({6-[(5-chloro-2-{4-[(1r,3r)-3-{7-[2-(2,6- dioxopiperidin-3-yl)-1-oxo-2,3-dihydro-1H- isoindol-5-yl]-2,7-diazaspiro[3.5]nonan-2- yl}cyclobutoxy]piperidin-1-yl}pyrimdiin-4- yl)amino]-2-oxo-1-(propan-2-yl)-1,2- dihydroquinolin-3-yl}oxy)-N- methylacetamide |
| 370 | 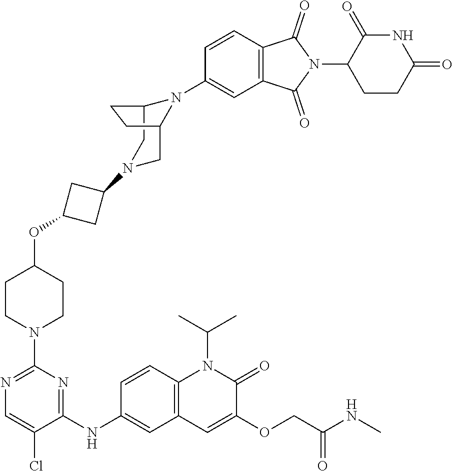 [SMILES: CNC(=O)COc1cc2cc(Nc3nc(N4CCC(O[C@H]5C[C@H](N6CC7CCC(C6)N7c6ccc7c(c6)C(=O)N(C6CCC(=O)NC6=O)C7=O)C5)CC4)ncc3Cl)ccc2n(C(C)C)c1=O] | 2-({6-[(5-chloro-2-{4-[(1r,3r)-3-{8-[2-(2,6- dioxopiperidin-3-yl)-1,3-dioxo-2,3-dihydro- 1H-isoindol-5-yl]-3,8- diazabicyclo[3.2.1]octan-3- yl}cyclobutoxy]piperidin-1-yl}pyrimidin-4- yl)amino]-2-oxo-1-(propan-2-yl)-1,2- dihydroquinolin-3-yl}oxy)-N- methylacetamide |
| 371 | 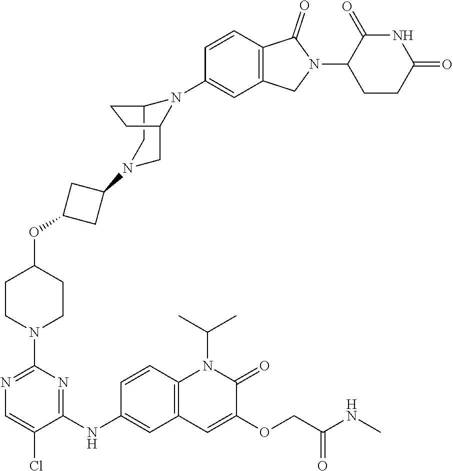 [SMILES: CNC(=O)COc1cc2cc(Nc3nc(N4CCC(O[C@H]5C[C@H](N6CC7CCC(C6)N7c6ccc7c(c6)CN(C6CCC(=O)NC6=O)C7=O)C5)CC4)ncc3Cl)ccc2n(C(C)C)c1=O] | 2-({6-[(5-chloro-2-{4-[(1r,3r)-3-{8-[2-(2,6- dioxopiperidin-3-yl)-1-oxo-2,3-dihydro-1H- isoindol-5-yl]-3,8-diazabicyclo[3.2.1]octan-3- yl}cyclobutoxy]piperidin-1-yl}pyrimidin-4- yl)amino]-2-oxo-1-(propan-2-yl)-1,2- dihydroquinolin-3-yl}oxy)-N- methylacetamide |
| 372 | 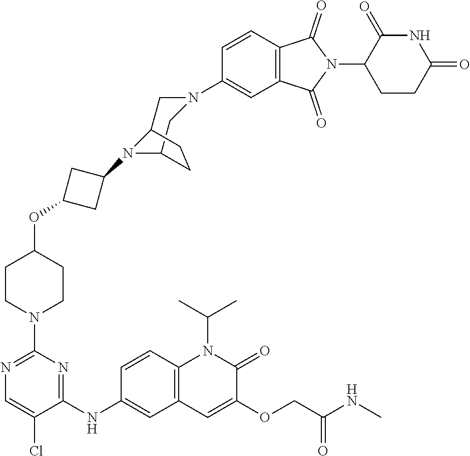 [SMILES: CNC(=O)COc1cc2cc(Nc3nc(N4CCC(O[C@H]5C[C@H](N6C7CCC6CN(c6ccc8c(c6)C(=O)N(C6CCC(=O)NC6=O)C8=O)C7)C5)CC4)ncc3Cl)ccc2n(C(C)C)c1=O] | 2-({6-[(5-chloro-2-{4-[(1r,3r)-3-{3-[2-(2,6- dioxopiperidin-3-yl)-1,3-dioxo-2,3-dihydro- 1H-isoindol-5-yl]-3,8- diazabicyclo[3.2.1]octan-8- yl}cyclobutoxy]piperidin-1-yl}pyrimidin-4- yl)amino]-2-oxo-1-(propan-2-yl)-1,2- dihydroquinolin-3-yl}oxy)-N- methylacetamide |
| 373 | 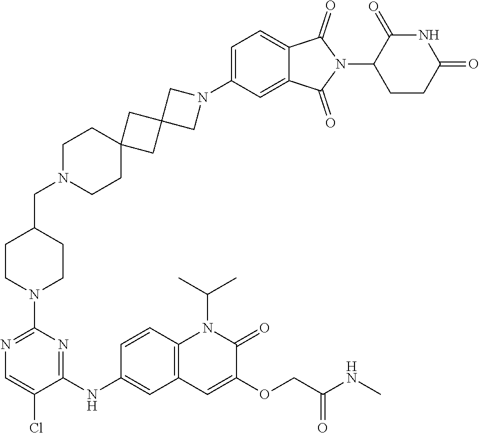 [SMILES: CNC(=O)COc1cc2cc(Nc3nc(N4CCC(CN5CCC6(CC5)CC5(CN(c7ccc8c(c7)C(=O)N(C7CCC(=O)NC7=O)C8=O)C5)C6)CC4)ncc3Cl)ccc2n(C(C)C)c1=O] | 2-{[6-({5-chloro-2-[4-({2-[2-(2,6- dioxopiperidin-3-yl)-1,3-dioxo-2,3-dihydro- 1H-isoindol-5-yl]-2,9- diazadispiro[3.1.5⁶.1⁴]dodecan-9- yl}methyl)piperidin-1-yl]pyrimidin-4- yl}amino)-2-oxo-1-(propan-2-yl)-1,2- dihydroquinolin-3-yl]oxy}-N- methylacetamide |
| 374 | 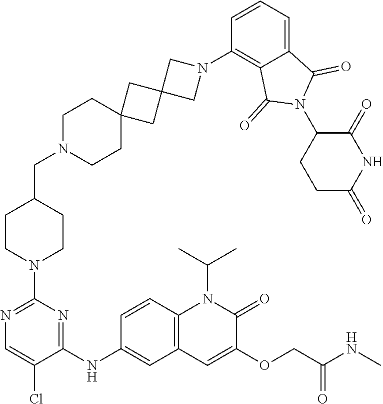 [SMILES: CNC(=O)COc1cc2cc(Nc3nc(N4CCC(CN5CCC6(CC5)CC5(CN(c7cccc8c7C(=O)N(C7CCC(=O)NC7=O)C8=O)C5)C6)CC4)ncc3Cl)ccc2n(C(C)C)c1=O] | 2-{[6-({5-chloro-2-[4-({2-[2-(2,6- dioxopiperidin-3-yl)-1,3-dioxo-2,3-dihydro- 1H-isoindol-4-yl]-2,9- diazadispiro[3.1.5⁶.1⁴]dodecan-9- yl}methyl)piperidin-1-yl]pyrimidin-4- yl}amino)-2-oxo-1-(propan-2-yl)-1,2- dihydroquinolin-3-yl]oxy}-N- methylacetamide |
| 375 | 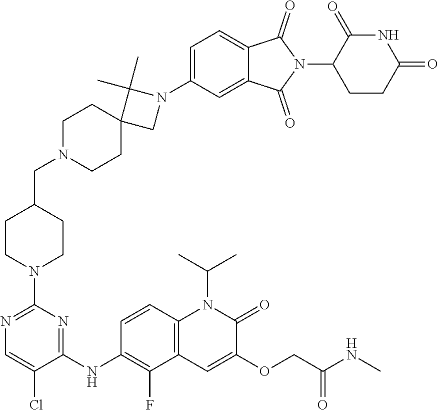 [SMILES: CNC(=O)COc1cc2c(F)c(Nc3nc(N4CCC(CN5CCC6(CC5)CN(c5ccc7c(c5)C(=O)N(C5CCC(=O)NC5=O)C7=O)C6(C)C)CC4)ncc3Cl)ccc2n(C(C)C)c1=O] | 2-{[6-({5-chloro-2-[4-({2-[2-(2,6- dioxopiperidin-3-yl)-1,3-dioxo-2,3-dihydro- 1H-isoindol-5-yl]-1,1-dimethyl-2,7- diazaspiro[3.5]nonan-7-yl}methyl)piperidin- 1-yl]pyrimidin-4-yl}amino)-5-fluoro-2-oxo-1- (propan-2-yl)-1,2-dihydroquinolin-3-yl]oxy}- N-methylacetamide |
| 376 | 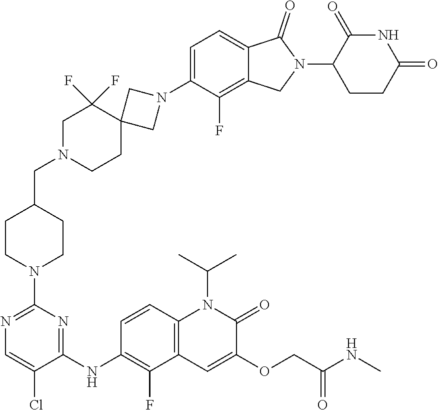 [SMILES: CNC(=O)COc1cc2c(F)c(Nc3nc(N4CCC(CN5CCC6(CN(c7ccc8c(c7F)CN(C7CCC(=O)NC7=O)C8=O)C6)C(F)(F)C5)CC4)ncc3Cl)ccc2n(C(C)C)c1=O] | 2-{[6-({5-chloro-2-[4-({2-[2-(2,6- dioxopiperidin-3-yl)-4-fluoro-1-oxo-2,3- dihydro-1H-isoindol-5-yl]-5,5-difluoro-2,7- diazaspiro[3.5]nonan-7-yl}methyl)piperidin- 1-yl]pyrimidin-4-yl}amino)-5-fluoro-2-oxo-1- (propan-2-yl)-1,2-dihydroquinolin-3-yl]oxy}- N-methylacetamide |
| 377 | 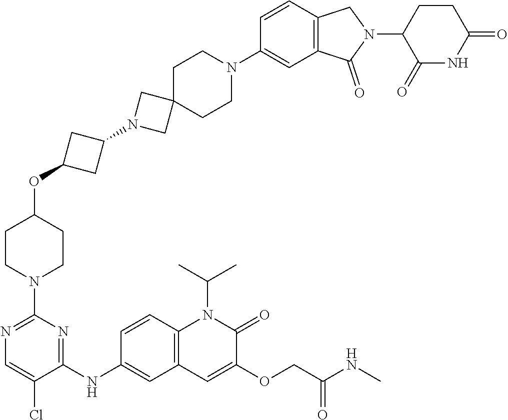 [SMILES: CNC(=O)COc1cc2cc(Nc3nc(N4CCC(O[C@H]5C[C@H](N6CC7(CCN(c8ccc9c(c8)C(=O)N(C8CCC(=O)NC8=O)C9)CC7)C6)C5)CC4)ncc3Cl)ccc2n(C(C)C)c1=O] | 2-({6-[(5-chloro-2-{4-[(1r,3r)-3-{7-[2-(2,6- dioxopiperidin-3-yl)-3-oxo-2,3-dihydro-1H- isoindol-5-yl]-2,7-diazaspiro[3.5]nonan-2- yl}cyclobutoxy]piperidin-1-yl}pyrimidin-4- yl)amino]-2-oxo-1-(propan-2-yl)-1,2- dihydroquinolin-3-yl}oxy)-N- methylacetamide |
| 378 | 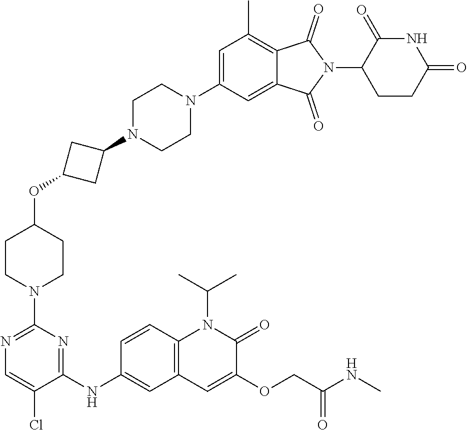 [SMILES: CNC(=O)COc1cc2cc(Nc3nc(N4CCC(O[C@H]5C[C@H](N6CCN(c7cc(C)c8c(c7)C(=O)N(C7CCC(=O)NC7=O)C8=O)CC6)C5)CC4)ncc3Cl)ccc2n(C(C)C)c1=O] | 2-({6-[(5-chloro-2-{4-[(1r,3r)-3-{4-[2-(2,6- dioxopiperidin-3-yl)-7-methyl-1,3-dioxo-2,3- dihydro-1H-isoindol-5-yl]piperazin-1- yl}cyclobutoxy]piperidin-1-yl}pyrimidin-4- yl)amino]-2-oxo-1-(propan-2-yl)-1,2- dihydroquinolin-3-yl}oxy)-N- methylacetamide |
| 379 | 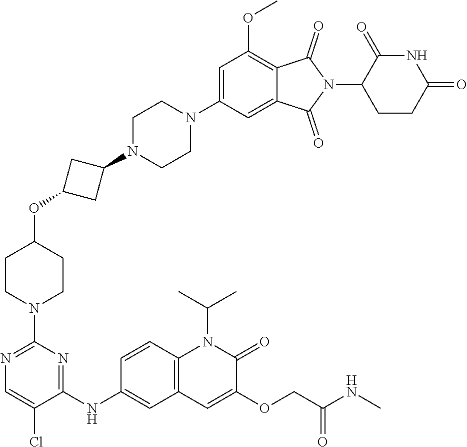 [SMILES: CNC(=O)COc1cc2cc(Nc3nc(N4CCC(O[C@H]5C[C@H](N6CCN(c7cc(OC)c8c(c7)C(=O)N(C7CCC(=O)NC7=O)C8=O)CC6)C5)CC4)ncc3Cl)ccc2n(C(C)C)c1=O] | 2-({6-[(5-chloro-2-{4-[(1r,3r)-3-{4-[2-(2,6- dioxopiperidin-3-yl)-7-methoxy-1,3-dioxo- 2,3-dihydro-1H-isoindol-5-yl]piperazin-1- yl}cyclobutoxy]piperidin-1-yl}pyrimidin-4- yl)amino]-2-oxo-1-(propan-2-yl)-1,2- dihydroquinolin-3-yl}oxy)-N- methylacetamide |
| 380 | 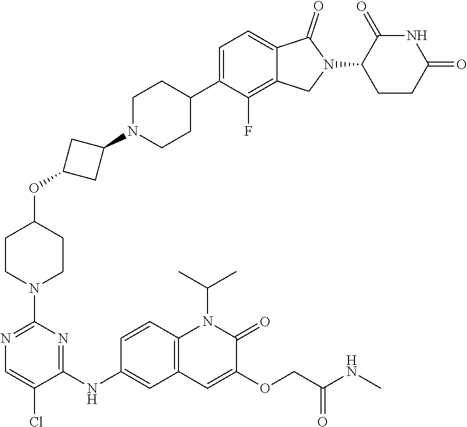 [SMILES: CNC(=O)COc1cc2cc(Nc3nc(N4CCC(O[C@H]5C[C@H](N6CCC(c7ccc8c(c7F)CN([C@H]7CCC(=O)NC7=O)C8=O)CC6)C5)CC4)ncc3Cl)ccc2n(C(C)C)c1=O] | 2-({6-[(5-chloro-2-{4-[(1r,3r)-3-(4-{2-[(3S)- 2,6-dioxopiperidin-3-yl]-4-fluoro-1-oxo-2,3- dihydro-1H-isoindol-5-yl}piperidin-1- yl)cyclobutoxy]piperidin-1-yl}pyrimidin-4- yl)amino]-5-fluoro-2-oxo-1-(propan-2-yl)- 1,2-dihydroquinolin-3-yl}oxy)-N- methylacetamide |
| 381 | 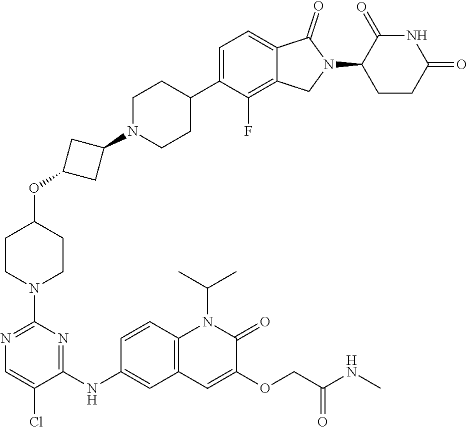 [SMILES: CNC(=O)COc1cc2cc(Nc3nc(N4CCC(O[C@H]5C[C@H](N6CCC(c7ccc8c(c7F)CN([C@@H]7CCC(=O)NC7=O)C8=O)CC6)C5)CC4)ncc3Cl)ccc2n(C(C)C)c1=O] | 2-({6-[(5-chloro-2-{4-[(1r,3r)-3-(4-{2-[(3R)- 2,6-dioxopiperidin-3-yl]-4-fluoro-1-oxo-2,3- dihydro-1H-isoindol-5-yl}piperidin-1- yl)cyclobutoxy]piperidin-1-yl}pyrimidin-4- yl)amino]-5-fluoro-2-oxo-1-(propan-2-yl)- 1,2-dihydroquinolin-3-yl}oxy)-N- methylacetamide |
| 382 | 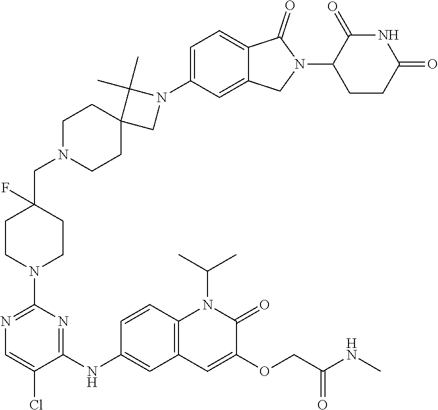 [SMILES: CNC(=O)COc1cc2cc(Nc3nc(N4CCC(F)(CN5CCC6(CC5)CN(c5ccc7c(c5)CN(C5CCC(=O)NC5=O)C7=O)C6(C)C)CC4)ncc3Cl)ccc2n(C(C)C)c1=O] | 2-{[6-({5-chloro-2-[4-({2-[2-(2,6- dioxopiperidin-3-yl)-1-oxo-2,3-dihydro-1H- isoindol-5-yl]-1,1-dimethyl-2,7- diazaspiro[3.5]nonan-7-yl}methyl)-4- fluoropiperidin-1-yl]pyrimidin-4-yl}amino)- 2-oxo-1-(propan-2-yl)-1,2-dihydroquinolin-3- yl]oxy}-N-methylacetamide |
| 383 | 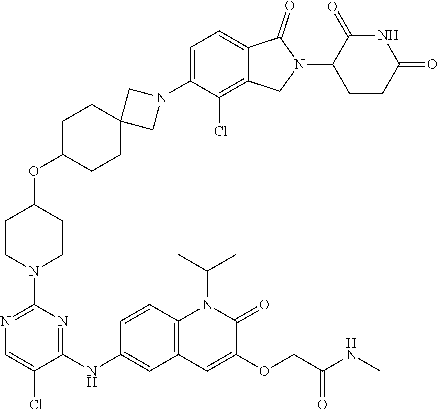 [SMILES: CNC(=O)COc1cc2cc(Nc3nc(N4CCC(OC5CCC6(CC5)CN(c5ccc7c(c5Cl)CN(C5CCC(=O)NC5=O)C7=O)C6)CC4)ncc3Cl)ccc2n(C(C)C)c1=O] | 2-{[6-({5-chloro-2-[4-({2-[4-chloro-2-(2,6- dioxopiperidin-3-yl)-1-oxo-2,3-dihydro-1H- isoindol-5-yl]-2-azaspiro[3.5]nonan-7- yl}oxy)piperidin-1-yl]pyrimidin-4-yl}amino)- 2-oxo-1-(propan-2-yl)-1,2-dihydroquinolin-3- yl]oxy}-N-methylacetamide |
| 384 | 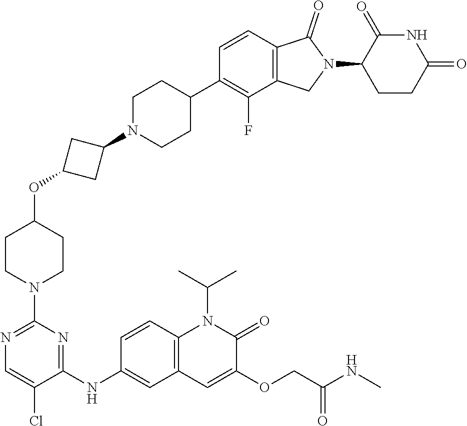 [SMILES: CNC(=O)COc1cc2cc(Nc3nc(N4CCC(O[C@H]5C[C@H](N6CCC(c7ccc8c(c7F)CN([C@@H]7CCC(=O)NC7=O)C8=O)CC6)C5)CC4)ncc3Cl)ccc2n(C(C)C)c1=O] | 2-({6-[(5-chloro-2-{4-[(1r,3r)-3-(4-{2-[(3R)- 2,6-dioxopiperidin-3-yl]-4-fluoro-1-oxo-2,3- dihydro-1H-isoindol-5-yl}piperidin-1- yl)cyclobutoxy]piperidin-1-yl}pyrimidin-4- yl)amino]-2-oxo-1-(propan-2-yl)-1,2- dihydroquinolin-3-yl}oxy)-N- methylacetamide |
| 385 | 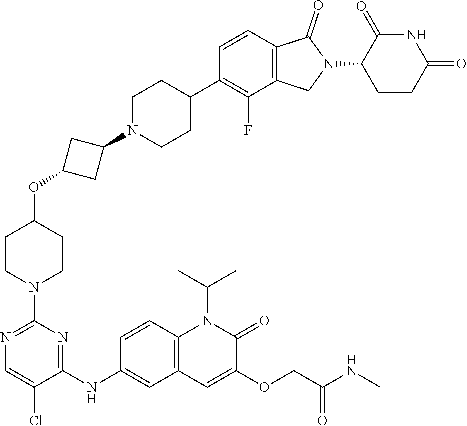 [SMILES: CNC(=O)COc1cc2cc(Nc3nc(N4CCC(O[C@H]5C[C@H](N6CCC(c7ccc8c(c7F)CN([C@H]7CCC(=O)NC7=O)C8=O)CC6)C5)CC4)ncc3Cl)ccc2n(C(C)C)c1=O] | 2-({6-[(5-chloro-2-{4-[(1r,3r)-3-(4-{2-[(3S)- 2,6-dioxopiperidin-3-yl]-4-fluoro-1-oxo-2,3- dihydro-1H-isoindol-5-yl}piperidin-1- yl)cyclobutoxy]piperidin-1-yl}pyrimidin-4- yl)amino]-2-oxo-1-(propan-2-yl)-1,2- dihydroquinolin-3-yl}oxy)-N- methylacetamide |
| 386 | 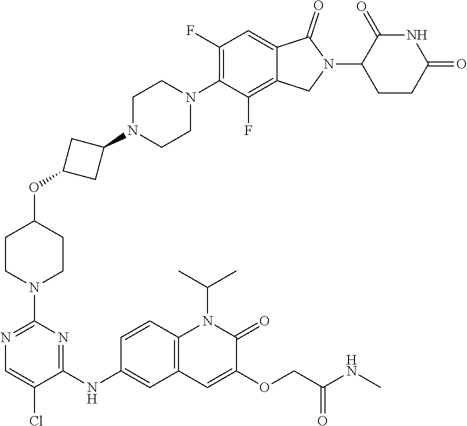 [SMILES: CNC(=O)COc1cc2cc(Nc3nc(N4CCC(O[C@H]5C[C@H](N6CCN(c7c(F)cc8c(c7F)CN(C7CCC(=O)NC7=O)C8=O)CC6)C5)CC4)ncc3Cl)ccc2n(C(C)C)c1=O] | 2-({6-[(5-chloro-2-{4-[(1r,3r)-3-{4-[2-(2,6- dioxopiperidin-3-yl)-4,6-difluoro-1-oxo-2,3- dihydro-1H-isoindol-5-yl]piperazin-1- yl}cyclobutoxy]piperidin-1-yl}pyrimidin-4- yl)amino]-2-oxo-1-(propan-2-yl)-1,2- dihydroquinolin-3-yl}oxy)-N- methylacetamide |
| 387 | 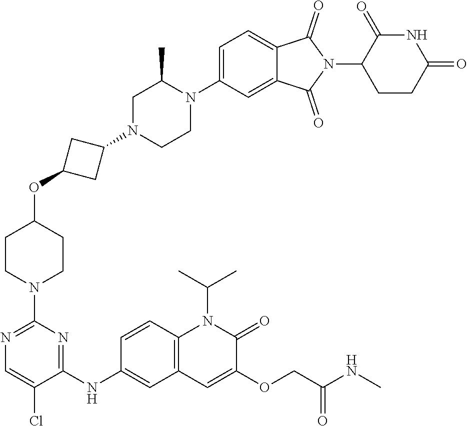 [SMILES: CNC(=O)COc1cc2cc(Nc3nc(N4CCC(O[C@H]5C[C@H](N6CCN(c7ccc8c(c7)C(=O)N(C7CCC(=O)NC7=O)C8=O)[C@H](C)C6)C5)CC4)ncc3Cl)ccc2n(C(C)C)c1=O] | 2-({6-[(5-chloro-2-{4-[(1r,3r)-3-[(3R)-4-[2- (2,6-dioxopiperidin-3-yl)-1,3-dioxo-2,3- dihydro-1H-isoindol-5-yl]-3-methylpiperazin- 1-yl]cyclobutoxy]piperidin-1-yl}pyrimidin-4- yl)amino]-2-oxo-1-(propan-2-yl)-1,2- dihydroquinolin-3-yl}oxy)-N- methylacetamide |
| 388 | 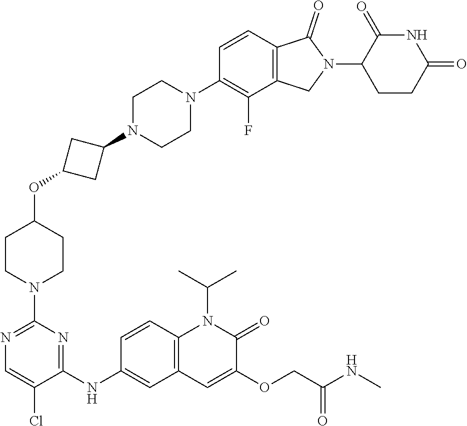 [SMILES: CNC(=O)COc1cc2cc(Nc3nc(N4CCC(O[C@H]5C[C@H](N6CCN(c7ccc8c(c7F)CN(C7CCC(=O)NC7=O)C8=O)CC6)C5)CC4)ncc3Cl)ccc2n(C(C)C)c1=O] | 2-({6-[(5-chloro-2-{4-[(1r,3r)-3-(4-{2-[(3S)- 2,6-dioxopiperidin-3-yl]-4-fluoro-1-oxo-2,3- dihydro-1H-isoindol-5-yl}piperazin-1- yl)cyclobutoxy]piperidin-1-yl}pyrimidin-4- yl)amino]-2-oxo-1-(propan-2-yl)-1,2- dihydroquinolin-3-yl}oxy)-N- methylacetamide |
| 389 | 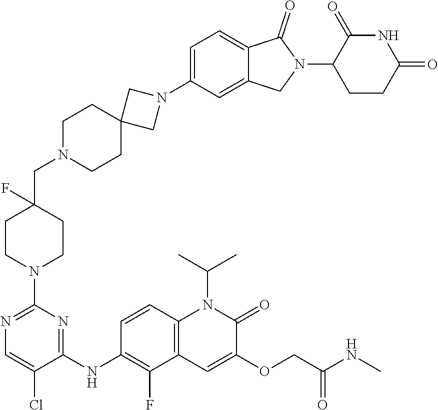 [SMILES: CNC(=O)COc1cc2c(F)c(Nc3nc(N4CCC(F)(CN5CCC6(CC5)CN(c5ccc7c(c5)CN(C5CCC(=O)NC5=O)C7=O)C6)CC4)ncc3Cl)ccc2n(C(C)C)c1=O] | 2-{[6-({5-chloro-2-[4-({2-[2-(2,6- dioxopiperidin-3-yl)-1-oxo-2,3-dihydro-1H- isoindol-5-yl]-2,7-diazaspiro[3.5]nonan-7- yl}methyl)-4-fluoropiperidin-1-yl]pyrimidin- 4-yl}amino)-5-fluoro-2-oxo-1-(propan-2-yl)- 1,2-dihydroquinolin-3-yl]oxy}-N- methylacetamide |
| 390 | 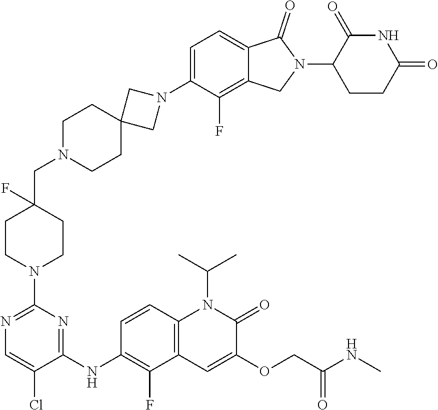 [SMILES: CNC(=O)COc1cc2c(F)c(Nc3nc(N4CCC(F)(CN5CCC6(CC5)CN(c5ccc7c(c5F)CN(C5CCC(=O)NC5=O)C7=O)C6)CC4)ncc3Cl)ccc2n(C(C)C)c1=O] | 2-{[6-({5-chloro-2-[4-({2-[2-(2,6- dioxopiperidin-3-yl)-4-fluoro-1-oxo-2,3- dihydro-1H-isoindol-5-yl]-2,7- diazaspiro[3.5]nonan-7-yl}methyl)-4- fluoropiperidin-1-yl]pyrimidin-4-yl}amino)- 5-fluoro-2-oxo-1-(propan-2-yl)-1,2- dihydroquinolin-3-yl]oxy}-N- methylacetamide |
| 391 | 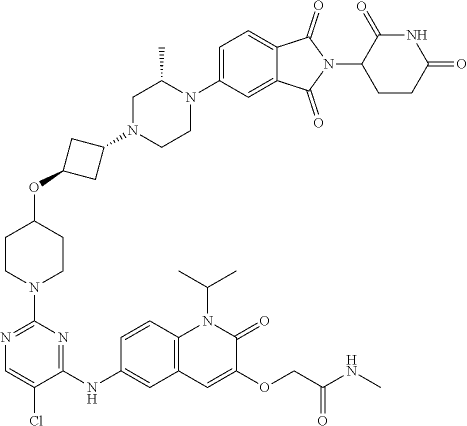 [SMILES: CNC(=O)COc1cc2cc(Nc3nc(N4CCC(O[C@H]5C[C@H](N6CCN(c7ccc8c(c7)C(=O)N(C7CCC(=O)NC7=O)C8=O)[C@@H](C)C6)C5)CC4)ncc3Cl)ccc2n(C(C)C)c1=O] | 2-({6-[(5-chloro-2-{4-[(1r,3r)-3-[(3S)-4-[2- (2,6-dioxopiperidin-3-yl)-1,3-dioxo-2,3- dihydro-1H-isoindol-5-yl]-3-methylpiperazin- 1-yl]cyclobutoxy]piperidin-1-yl}pyrimidin-4- yl)amino]-2-oxo-1-(propan-2-yl)-1,2- dihydroquinolin-3-yl}oxy)-N- methylacetamide |
| 392 | 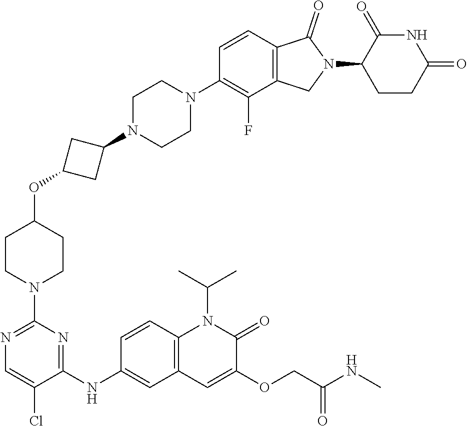 [SMILES: CNC(=O)COc1cc2cc(Nc3nc(N4CCC(O[C@H]5C[C@H](N6CCN(c7ccc8c(c7F)CN([C@@H]7CCC(=O)NC7=O)C8=O)CC6)C5)CC4)ncc3Cl)ccc2n(C(C)C)c1=O] | 2-({6-[(5-chloro-2-{4-[(1r,3r)-3-(4-{2-[(3R)- 2,6-dioxopiperidin-3-yl]-4-fluoro-1-oxo-2,3- dihydro-1H-isoindol-5-yl}piperazin-1- yl)cyclobutoxy]piperidin-1-yl}pyrimidin-4- yl)amino]-2-oxo-1-(propan-2-yl)-1,2- dihydroquinolin-3-yl}oxy)-N- methylacetamide |
| 393 | 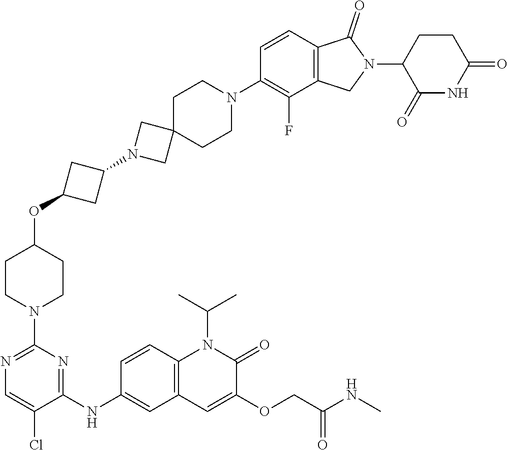 [SMILES: CNC(=O)COc1cc2cc(Nc3nc(N4CCC(O[C@H]5C[C@H](N6CC7(CCN(c8ccc9c(c8F)CN(C8CCC(=O)NC8=O)C9=O)CC7)C6)C5)CC4)ncc3Cl)ccc2n(C(C)C)c1=O] | 2-({6-[(5-chloro-2-{4-[(1r,3r)-3-{7-[2-(2,6- dioxopiperidin-3-yl)-4-fluoro-1-oxo-2,3- dihydro-1H-isoindol-5-yl]-2,7- diazaspiro[3.5]nonan-2- yl}cyclobutoxy]piperidin-1-yl}pyrimidin-4- yl)amino]-2-oxo-1-(propan-2-yl)-1,2- dihydroquinolin-3-yl}oxy)-N- methylacetamide |
| 394 | 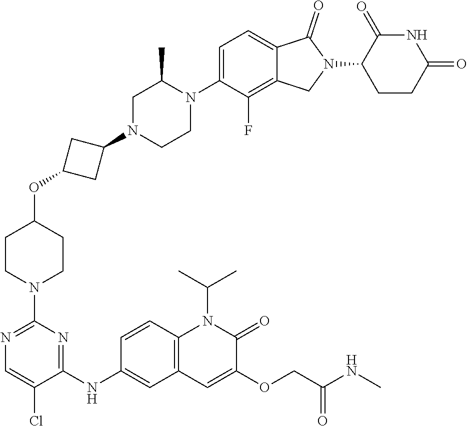 [SMILES: CNC(=O)COc1cc2cc(Nc3nc(N4CCC(O[C@H]5C[C@H](N6CCN(c7ccc8c(c7F)CN([C@H]7CCC(=O)NC7=O)C8=O)[C@H](C)C6)C5)CC4)ncc3Cl)ccc2n(C(C)C)c1=O] | 2-({6-[(5-chloro-2-{4-[(1r,3r)-3-[(3R)-4-{2- [(3S)-2,6-dioxopiperidin-3-yl]-4-fluoro-1- oxo-2,3-dihydro-1H-isoindol-5-yl}-3- methylpiperazin-1-yl]cyclobutoxy]piperidin- 1-yl}pyrimidin-4-yl)amino]-2-oxo-1-(propan- 2-yl)-1,2-dihydroquinolin-3-yl}oxy)-N- methylacetamide |
| 395 | 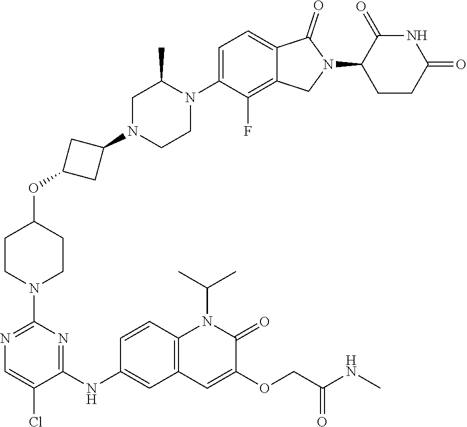 [SMILES: CNC(=O)COc1cc2cc(Nc3nc(N4CCC(O[C@H]5C[C@H](N6CCN(c7ccc8c(c7F)CN([C@@H]7CCC(=O)NC7=O)C8=O)[C@H](C)C6)C5)CC4)ncc3Cl)ccc2n(C(C)C)c1=O] | 2-({6-[(5-chloro-2-{4-[(1r,3r)-3-[(3R)-4-{2- [(3R)-2,6-dioxopiperidin-3-yl]-4-fluoro-1- oxo-2,3-dihydro-1H-isoindol-5-yl}-3- methylpiperazin-1-yl]cyclobutoxy]piperidin- 1-yl}pyrimidin-4-yl)amino]-2-oxo-1-(propan- 2-yl)-1,2-dihydroquinolin-3-yl}oxy)-N- methylacetamide |
| 396 | 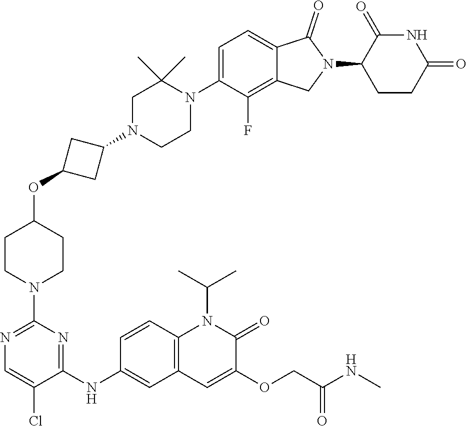 [SMILES: CNC(=O)COc1cc2cc(Nc3nc(N4CCC(O[C@H]5C[C@H](N6CCN(c7ccc8c(c7F)CN([C@@H]7CCC(=O)NC7=O)C8=O)C(C)(C)C6)C5)CC4)ncc3Cl)ccc2n(C(C)C)c1=O] | 2-({6-[(5-chloro-2-{4-[(1r,3r)-3-[(4R)-4-{2- [(3R)-2,6-dioxopiperidin-3-yl]-4-fluoro-1- oxo-2,3-dihydro-1H-isoindol-5-yl}-3,3- dimethylpiperidin-1- yl]cyclobutoxy]piperidin-1-yl}pyrimidin-4- yl)amino]-2-oxo-1-(propan-2-yl)-1,2- dihydroquinolin-3-yl}oxy)-N- methylacetamide |
| 397 | 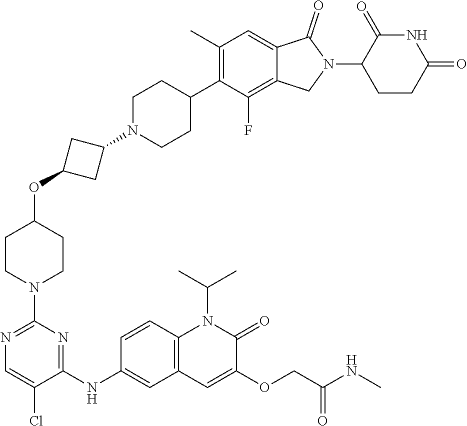 [SMILES: CNC(=O)COc1cc2cc(Nc3nc(N4CCC(O[C@H]5C[C@H](N6CCC(c7c(C)cc8c(c7F)CN(C7CCC(=O)NC7=O)C8=O)CC6)C5)CC4)ncc3Cl)ccc2n(C(C)C)c1=O] | 2-({6-[(5-chloro-2-{4-[(1r,3r)-3-{4-[2-(2,6- dioxopiperidin-3-yl)-4-fluoro-6-methyl-1- oxo-2,3-dihydro-1H-isoindol-5-yl]piperidin- 1-yl}cyclobutoxy]piperidin-1-yl}pyrimidin-4- yl)amino]-2-oxo-1-(propan-2-yl)-1,2- dihydroquinolin-3-yl}oxy)-N- methylacetamide |
| 398 | 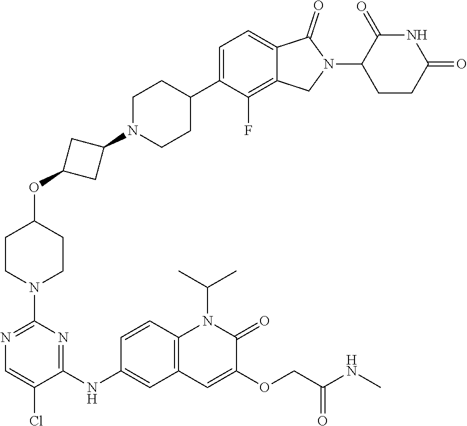 [SMILES: CNC(=O)COc1cc2cc(Nc3nc(N4CCC(O[C@H]5C[C@@H](N6CCC(c7ccc8c(c7F)CN(C7CCC(=O)NC7=O)C8=O)CC6)C5)CC4)ncc3Cl)ccc2n(C(C)C)c1=O] | 2-({6-[(5-chloro-2-{4-[(1s,3s)-3-{4-[2-(2,6- dioxopiperidin-3-yl)-4-fluoro-1-oxo-2,3- dihydro-1H-isoindol-5-yl]piperidin-1- yl}cyclobutoxy]piperidin-1-yl}pyrimidin-4- yl)amino]-2-oxo-1-(propan-2-yl)-1,2- dihydroquinolin-3-yl}oxy)-N- methylacetamide |
| 399 | 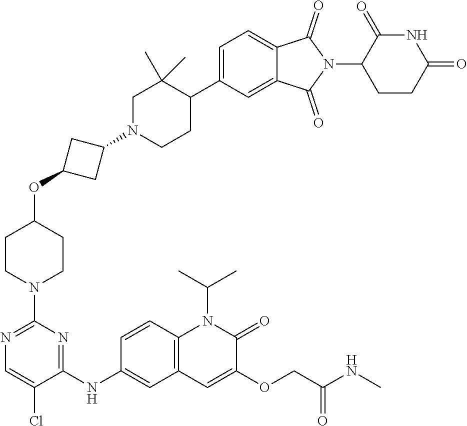 [SMILES: CNC(=O)COc1cc2cc(Nc3nc(N4CCC(O[C@H]5C[C@H](N6CCC(c7ccc8c(c7)C(=O)N(C7CCC(=O)NC7=O)C8=O)C(C)(C)C6)C5)CC4)ncc3Cl)ccc2n(C(C)C)c1=O] | 2-({6-[(5-chloro-2-{4-[(1r,3r)-3-{4-[2-(2,6- dioxopiperidin-3-yl)-1,3-dioxo-2,3-dihydro- 1H-isoindol-5-yl]-3,3-dimethylpiperidin-1- yl}cyclobutoxy]piperidin-1-yl}pyrimidin-4- yl)amino]-2-oxo-1-(propan-2-yl)-1,2- dihydroquinolin-3-yl}oxy)-N- methylacetamide |
| 400 | 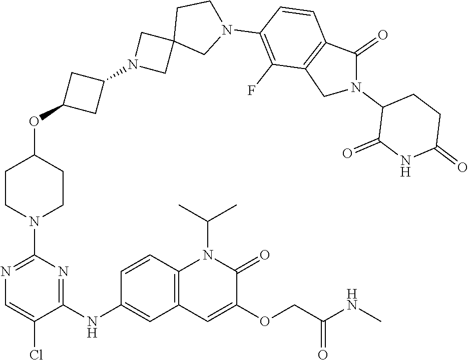 [SMILES: CNC(=O)COc1cc2cc(Nc3nc(N4CCC(O[C@H]5C[C@H](N6CC7(CCN(c8ccc9c(c8F)CN(C8CCC(=O)NC8=O)C9=O)C7)C6)C5)CC4)ncc3Cl)ccc2n(C(C)C)c1=O] | 2-({6-[(5-chloro-2-{4-[(1r,3r)-3-{6-[2-(2,6- dioxopiperidin-3-yl)-4-fluoro-1-oxo-2,3- dihydro-1H-isoindol-5-yl]-2,6- diazaspiro[3.4]octan-2- yl}cyclobutoxy]piperidin-1-yl}pyrimdiin-4- yl)amino]-2-oxo-1-(propan-2-yl)-1,2- dihydroquinolin-3-yl}oxy)-N- methylacetamide |
| 401 | 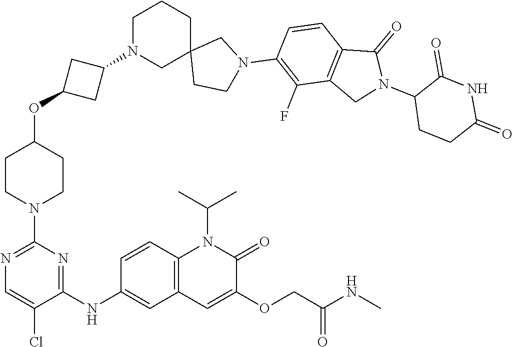 [SMILES: CNC(=O)COc1cc2cc(Nc3nc(N4CCC(O[C@H]5C[C@H](N6CCCC7(CCN(c8ccc9c(c8F)CN(C8CCC(=O)NC8=O)C9=O)C7)C6)C5)CC4)ncc3Cl)ccc2n(C(C)C)c1=O] | 2-({6-[(5-chloro-2-{4-[(1r,3r)-3-{2-[2-(2,6- dioxopiperidin-3-yl)-4-fluoro-1-oxo-2,3- dihydro-1H-isoindol-5-yl]-2,7- diazaspiro[4.5]decan-7- yl}cyclobutoxy]piperidin-1-yl}pyrimidin-4- yl)amino]-2-oxo-1-(propan-2-yl)-1,2- dihydroquinolin-3-yl}oxy)-N- methylacetamide |
| 402 | 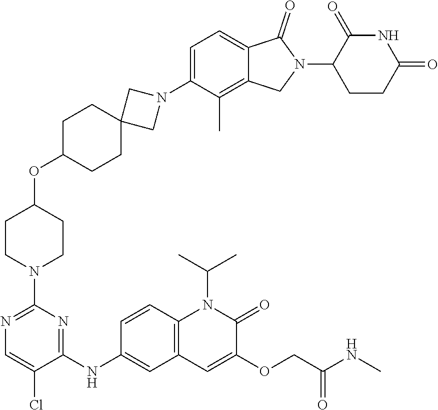 [SMILES: CNC(=O)COc1cc2cc(Nc3nc(N4CCC(OC5CCC6(CC5)CN(c5ccc7c(c5C)CN(C5CCC(=O)NC5=O)C7=O)C6)CC4)ncc3Cl)ccc2n(C(C)C)c1=O] | 2-{[6-({5-chloro-2-[4-({2-[2-(2,6- dioxopiperidin-3-yl)-4-methyl-1-oxo-2,3- dihydro-1H-isoindol-5-yl]-2- azaspiro[3.5]nonan-7-yl}oxy)piperidin-1- yl]pyrimidin-4-yl}amino)-2-oxo-1-(propan-2- yl)-1,2-dihydroquinolin-3-yl]oxy}-N- methylacetamide |
| 403 | 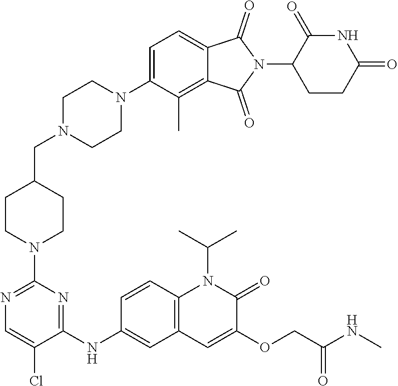 [SMILES: CNC(=O)COc1cc2cc(Nc3nc(N4CCC(CN5CCN(c6ccc7c(c6C)C(=O)N(C6CCC(=O)NC6=O)C7=O)CC5)CC4)ncc3Cl)ccc2n(C(C)C)c1=O] | 2-{[6-({5-chloro-2-[4-({4-[2-(2,6- dioxopiperidin-3-yl)-4-methyl-1,3-dioxo-2,3- dihydro-1H-isoindol-5-yl]piperazin-1- yl}methyl)piperidin-1-yl]pyrimidin-4- yl}amino)-2-oxo-1-(proapn-2-yl)-1,2- dihydroquinolin-3-yl]oxy}-N- methylacetamide |
| 404 | 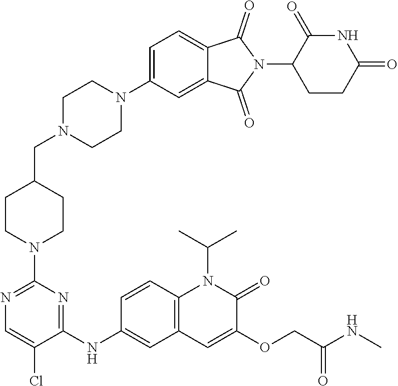 [SMILES: CNC(=O)COc1cc2cc(Nc3nc(N4CCC(CN5CCN(c6ccc7c(c6)C(=O)N(C6CCC(=O)NC6=O)C7=O)CC5)CC4)ncc3Cl)ccc2n(C(C)C)c1=O] | 2-{[6-({5-chloro-2-[4-({4-[2-(2,6- dioxopiperidin-3-yl)-1,3-dioxo-2,3-dihydro- 1H-isoindol-5-yl]piperazin-1- yl}methyl)piperidin-1-yl]pyrimidin-4- yl}amino)-2-oxo-1-(propan-2-yl)-1,2- dihydroquinolin-3-yl]oxy}-N- methylacetamide |
| 405 | 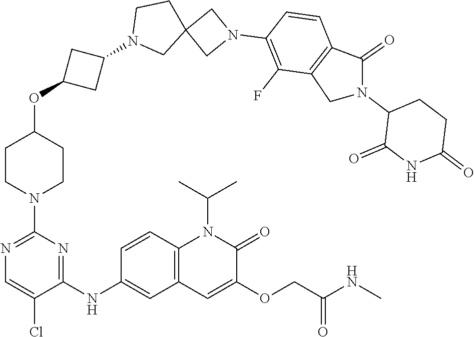 [SMILES: CNC(=O)COc1cc2cc(Nc3nc(N4CCC(O[C@H]5C[C@H](N6CCC7(CN(c8ccc9c(c8F)CN(C8CCC(=O)NC8=O)C9=O)C7)C6)C5)CC4)ncc3Cl)ccc2n(C(C)C)c1=O] | 2-({6-[(5-chloro-2-{4-[(1r,3r)-3-{2-[2-(2,6- dioxopiperidin-3-yl)-4-fluoro-1-oxo-2,3- dihydro-1H-isoindol-5-yl]-2,6- diazaspiro[3.4]octan-6- yl}cyclobutoxy]piperidin-1-yl}pyrimidin-4- yl)amino]-2-oxo-1-(propan-2-yl)-1,2- dihydroquinolin-3-yl}oxy)-N- methylacetamide |
| 406 | 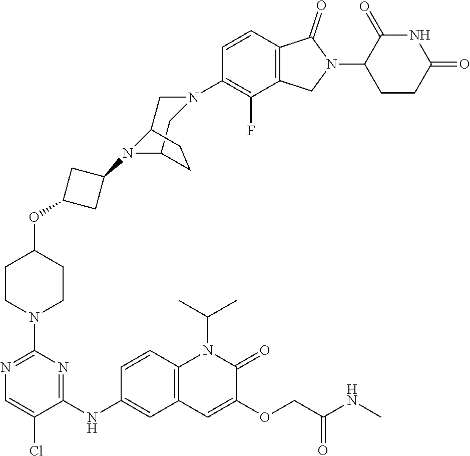 [SMILES: CNC(=O)COc1cc2cc(Nc3nc(N4CCC(O[C@H]5C[C@H](N6C7CCC6CN(c6ccc8c(c6F)CN(C6CCC(=O)NC6=O)C8=O)C7)C5)CC4)ncc3Cl)ccc2n(C(C)C)c1=O] | 2-({6-[(5-chloro-2-{4-[(1r,3r)-3-{3-[2-(2,6- dioxopiperidin-3-yl)-4-fluoro-1-oxo-2,3- dihydro-1H-isoindol-5-yl]-3,8- diazabicyclo[3.2.1]octan-8- yl}cyclobutoxy]piperidin-1-yl}pyrimidin-5- yl)amino]-2-oxo-1-(propan-2-yl)-1,2- dihydroquinolin-3-yl}oxy)-N- methylacetamide |
| 407 | 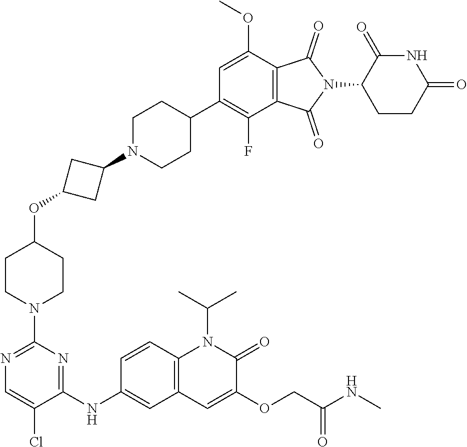 [SMILES: CNC(=O)COc1cc2cc(Nc3nc(N4CCC(O[C@H]5C[C@H](N6CCC(c7cc(OC)c8c(c7F)C(=O)N([C@H]7CCC(=O)NC7=O)C8=O)CC6)C5)CC4)ncc3Cl)ccc2n(C(C)C)c1=O] | 2-({6-[(5-chloro-2-{4-[(1r,3r)-3-(4-{2-[(3S)- 2,6-dioxopiperidin-3-yl]-4-fluoro-7-methoxy- 1-oxo-2,3-dihydro-1H-isoindol-5- yl}piperidin-1-yl)cyclobutoxy]piperidin-1- yl}pyrimidin-4-yl)amino]-2-oxo-1-(propan-2- yl)-1,2-dihydroquinolin-3-yl}oxy)-N- methylacetamide |
| 408 | 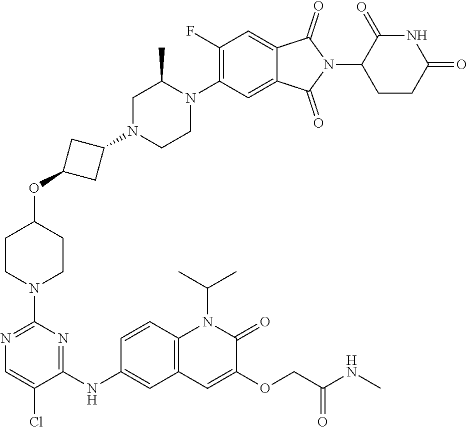 [SMILES: CNC(=O)COc1cc2cc(Nc3nc(N4CCC(O[C@H]5C[C@H](N6CCN(c7cc8c(cc7F)C(=O)N(C7CCC(=O)NC7=O)C8=O)[C@H](C)C6)C5)CC4)ncc3Cl)ccc2n(C(C)C)c1=O] | 2-({6-[(5-chloro-2-{4-[(1r,3r)-3-[(3R)-4-[2- (2,6-dioxopiperidin-3-yl)-6-fluoro-1,3-dioxo- 2,3-dihydro-1H-isoindol-5-yl]-3- methylpiperazin-1-yl]cyclobutoxy]piperidin- 1-yl}pyrimidin-4-yl)amino]-2-oxo-1-(propan- 2-yl)-1,2-dihydroquinolin-3-yl}oxy)-N- methylacetamide |
| 409 | 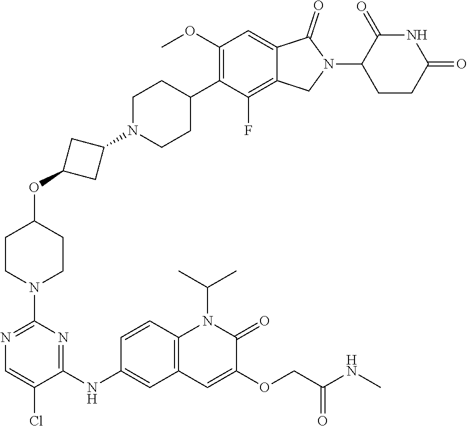 [SMILES: CNC(=O)COc1cc2cc(Nc3nc(N4CCC(O[C@H]5C[C@H](N6CCC(c7c(OC)cc8c(c7F)CN(C7CCC(=O)NC7=O)C8=O)CC6)C5)CC4)ncc3Cl)ccc2n(C(C)C)c1=O] | 2-({6-[(5-chloro-2-{4-[(1r,3r)-3-{4-[2-(2,6- dioxopiperidin-3-yl)-4-fluoro-6-methoxy-1- oxo-2,3-dihydro-1H-isoindol-5-yl]piperidin- 1-yl}cyclobutoxy]piperidin-1-yl}pyrimidin-4- yl)amino]-2-oxo-1-(propan-2-yl)-1,2- dihydroquinolin-3-yl}oxy)-N- methylacetamide |
| 410 | 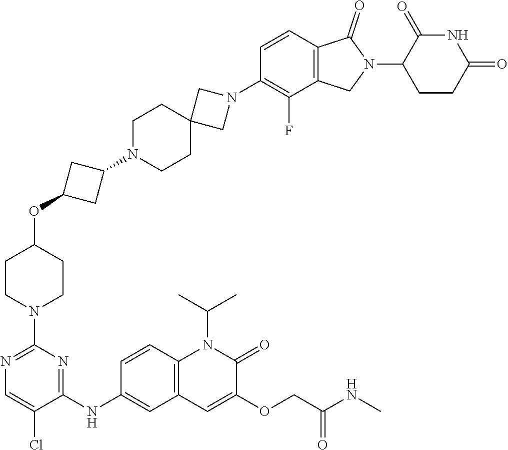 [SMILES: CNC(=O)COc1cc2cc(Nc3nc(N4CCC(O[C@H]5C[C@H](N6CCC7(CC6)CN(c6ccc8c(c6F)CN(C6CCC(=O)NC6=O)C8=O)C7)C5)CC4)ncc3Cl)ccc2n(C(C)C)c1=O] | 2-({6-[(5-chloro-2-{4-[(1r,3r)-3-{2-[2-(2,6- dioxopiperidin-3-yl)-4-fluoro-1-oxo-2,3- dihydro-1H-isoindol-5-yl]-2,7- diazaspiro[3.5]nonan-7- yl}cyclobutoxy]piperidin-1-yl}pyrimidin-4- yl)amino]-2-oxo-1-(propan-2-yl)-1,2- dihydroquinolin-3-yl}oxy)-N- methylacetamide |
| 411 | 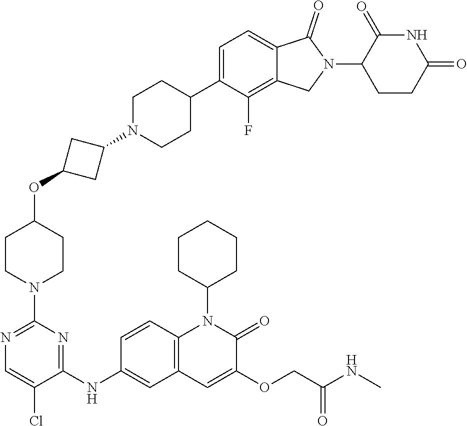 [SMILES: CNC(=O)COc1cc2cc(Nc3nc(N4CCC(O[C@H]5C[C@H](N6CCC(c7ccc8c(c7F)CN(C7CCC(=O)NC7=O)C8=O)CC6)C5)CC4)ncc3Cl)ccc2n(C2CCCCC2)c1=O] | 2-({6-[(5-chloro-2-{4-[(1r,3r)-3-{4-[2-(2,6- dioxopiperidin-3-yl)-4-fluoro-1-oxo-2,3- dihydro-1H-isoindol-5-yl]piperidin-1- yl}cyclobutoxy]piperidin-1-yl}pyrimidin-4- yl)amino]-1-cyclohexyl-2-oxo-1,2- dihydroquinolin-3-yl}oxy)-N- methylacetamide |
| 412 | 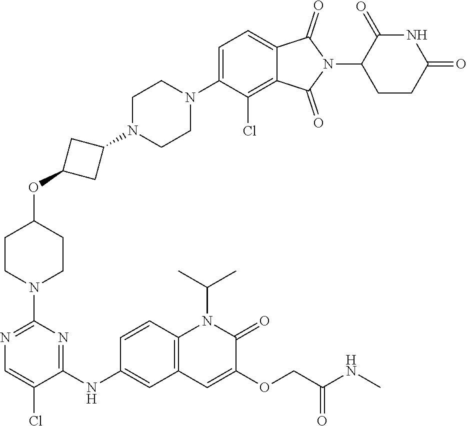 [SMILES: CNC(=O)COc1cc2cc(Nc3nc(N4CCC(O[C@H]5C[C@H](N6CCN(c7ccc8c(c7Cl)C(=O)N(C7CCC(=O)NC7=O)C8=O)CC6)C5)CC4)ncc3Cl)ccc2n(C(C)C)c1=O] | 2-({6-[(5-chloro-2-{4-[(1r,3r)-3-{4-[4-chloro- 2-(2,6-dioxopiperidin-3-yl)-1,3-dioxo-2,3- dihydro-1H-isoindol-5-yl]piperazin-1- yl}cyclobutoxy]piperidin-1-yl}pyrimidin-4- yl)amino]-2-oxo-1-(propan-2-yl)-1,2- dihydroquinolin-3-yl}oxy)-N- methylacetamide |
| 413 | 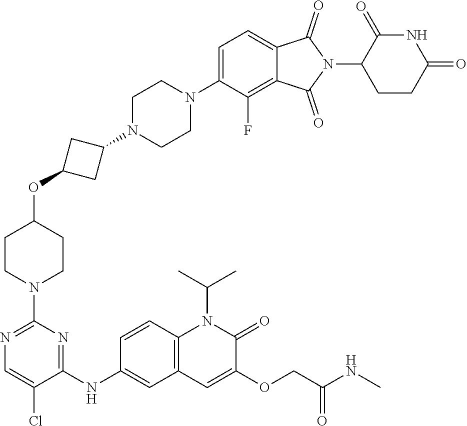 [SMILES: CNC(=O)COc1cc2cc(Nc3nc(N4CCC(O[C@H]5C[C@H](N6CCN(c7ccc8c(c7F)C(=O)N(C7CCC(=O)NC7=O)C8=O)CC6)C5)CC4)ncc3Cl)ccc2n(C(C)C)c1=O] | 2-({6-[(5-chloro-2-{4-[(1r,3r)-3-{4-[2-(2,6- dioxopiperidin-3-yl)-4-fluoro-1,3-dioxo-2,3- dihydro-1H-isoindol-5-yl]piperazin-1- yl}cyclobutoxy]piperidin-1-yl}pyrimidin-4- yl)amino]-2-oxo-1-(propan-2-yl)-1,2- dihydroquinolin-3-yl}oxy)-N- methylacetamide |
| 414 | 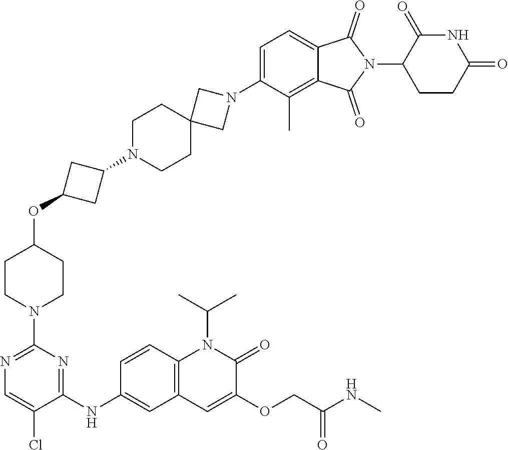 [SMILES: CNC(=O)COc1cc2cc(Nc3nc(N4CCC(O[C@H]5C[C@H](N6CCC7(CC6)CN(c6ccc8c(c6C)C(=O)N(C6CCC(=O)NC6=O)C8=O)C7)C5)CC4)ncc3Cl)ccc2n(C(C)C)c1=O] | 2-({6-[(5-chloro-2-{4-[(1r,3r)-3-{2-[2-(2,6- dioxopiperidin-3-yl)-4-methyl-1,3-dioxo-2,3- dihydro-1H-isoindol-5-yl]-2,7- diazaspiro[3.5]nonan-7- yl}cyclobutoxy]piperidin-1-yl}pyrimidin-4- yl)amino]-2-oxo-1-(propan-2-yl)-1,2- dihydroquinolin-3-yl}oxy)-N- methylacetamide |
| 415 | 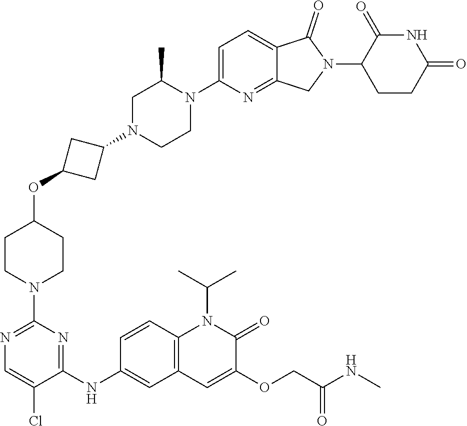 [SMILES: CNC(=O)COc1cc2cc(Nc3nc(N4CCC(O[C@H]5C[C@H](N6CCN(c7ccc8c(n7)CN(C7CCC(=O)NC7=O)C8=O)[C@H](C)C6)C5)CC4)ncc3Cl)ccc2n(C(C)C)c1=O] | 2-({6-[(5-chloro-2-{4-[(1r,3r)-3-[(3R)-4-[6- (2,6-dioxopiperidin-3-yl)-5-oxo-5H,6H,7H- pyrrolo[3,4-b]pyridin-2-yl]-3- methylpiperazin-1-yl]cyclobutoxy]piperidin- 1-yl}pyrimidin-4-yl)amino]-2-oxo-1-(propan- 2-yl)-1,2-dihydroquinolin-3-yl}oxy)-N- methylacetamide |
| 416 | 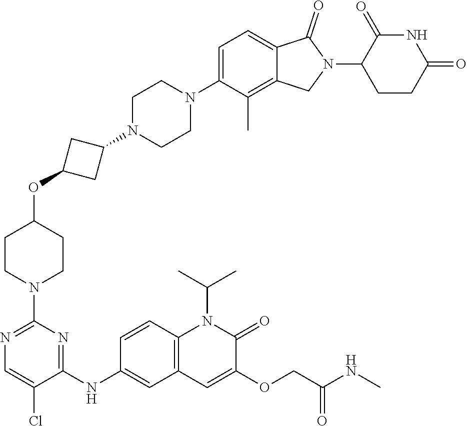 [SMILES: CNC(=O)COc1cc2cc(Nc3nc(N4CCC(O[C@H]5C[C@H](N6CCN(c7ccc8c(c7C)CN(C7CCC(=O)NC7=O)C8=O)CC6)C5)CC4)ncc3Cl)ccc2n(C(C)C)c1=O] | 2-({6-[(5-chloro-2-{4-[(1r,3r)-3-{4-[2-(2,6- dioxopiperidin-3-yl)-4-methyl-1-oxo-2,3- dihydro-1H-isoindol-5-yl]piperazin-1- yl}cyclobutoxy]piperidin-1-yl}pyrimidin-4- yl)amino]-2-oxo-1-(propan-2-yl)-1,2- dihydroquinolin-3-yl}oxy)-N- methylacetamide |
| 417 | 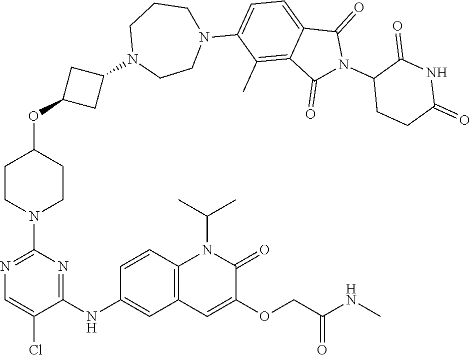 [SMILES: CNC(=O)COc1cc2cc(Nc3nc(N4CCC(O[C@H]5C[C@H](N6CCCN(c7ccc8c(c7C)C(=O)N(C7CCC(=O)NC7=O)C8=O)CC6)C5)CC4)ncc3Cl)ccc2n(C(C)C)c1=O] | 2-({6-[(5-chloro-2-{4-[(1r,3r)-3-{4-[2-(2,6- dioxopiperidin-3-yl)-4-methyl-1,3-dioxo-2,3- dihydro-1H-isoindol-5-yl]-1,4-diazepan-1- yl}cyclobutoxy]piperidin-1-yl}pyrimidin-4- yl)amino]-2-oxo-1-(propan-2-yl)-1,2- dihydroquinolin-3-yl}oxy)-N- methylacetamide |
| 418 | 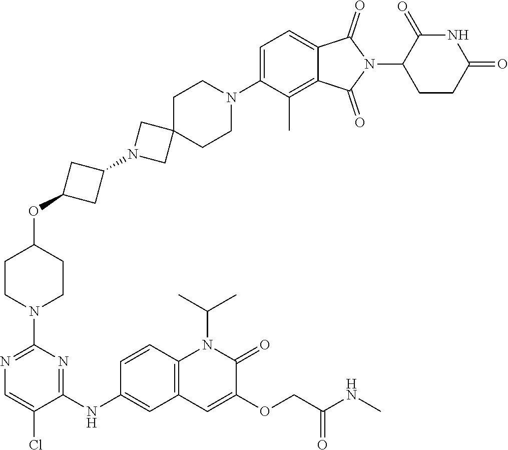 [SMILES: CNC(=O)COc1cc2cc(Nc3nc(N4CCC(O[C@H]5C[C@H](N6CC7(CCN(c8ccc9c(c8C)C(=O)N(C8CCC(=O)NC8=O)C9=O)CC7)C6)C5)CC4)ncc3Cl)ccc2n(C(C)C)c1=O] | 2-({6-[(5-chloro-2-{4-[(1r,3r)-3-{7-[2-(2,6- dioxopiperidin-3-yl)-4-methyl-1,3-dioxo-2,3- dihydro-1H-isoindol-5-yl]-2,7- diazaspiro[3.5]nonan-2- yl}cyclobutoxy]piperidin-1-yl}pyrimidin-4- yl)amino]-2-oxo-1-(propan-2-yl)-1,2- dihydroquinolin-3-yl}oxy)-N- methylacetamide |
| 419 | 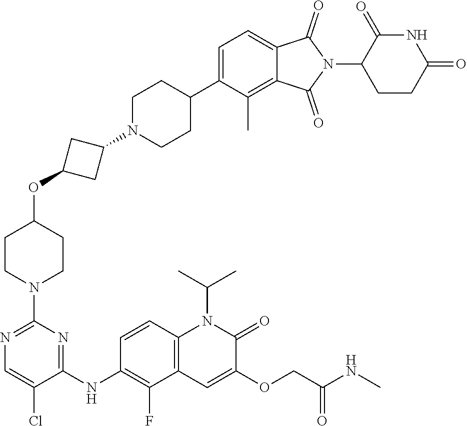 [SMILES: CNC(=O)COc1cc2c(F)c(Nc3nc(N4CCC(O[C@H]5C[C@H](N6CCC(c7ccc8c(c7C)C(=O)N(C7CCC(=O)NC7=O)C8=O)CC6)C5)CC4)ncc3Cl)ccc2n(C(C)C)c1=O] | 2-({6-[(5-chloro-2-{4-[(1r,3r)-3-{4-[2-(2,6- dioxopiperidin-3-yl)-4-methyl-1,3-dioxo-2,3- dihydro-1H-isoindol-5-yl]piperidin-1- yl}cyclobutoxy]piperidin-1-yl}pyrimidin-4- yl)amino]-5-fluoro-2-oxo-1-(propan-2-yl)- 1,2-dihydroquinolin-3-yl}oxy)-N- methylacetamide |
| 420 | 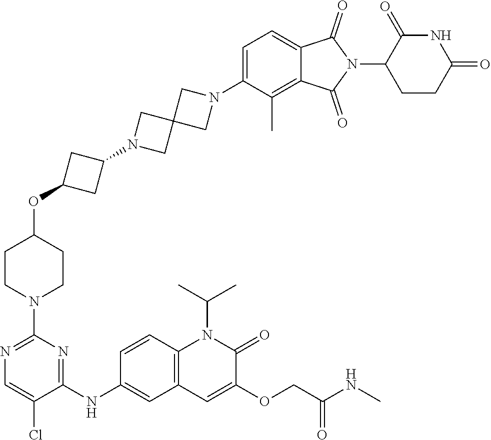 [SMILES: CNC(=O)COc1cc2cc(Nc3nc(N4CCC(O[C@H]5C[C@H](N6CC7(CN(c8ccc9c(c8C)C(=O)N(C8CCC(=O)NC8=O)C9=O)C7)C6)C5)CC4)ncc3Cl)ccc2n(C(C)C)c1=O] | 2-({6-[(5-chloro-2-{4-[(1r,3r)-3-{6-[2-(2,6- dioxopiperidin-3-yl)-4-methyl-1,3-dioxo-2,3- dihydro-1H-isoindol-5-yl]-2,6- diazaspiro[3.3]heptan-2- yl}cyclobutoxy]piperidin-1-yl}pyrimidin-4- yl)amino]-2-oxo-1-(propan-2-yl)-1,2- dihydroquinolin-3-yl}oxy)-N- methylacetamide |
| 421 | 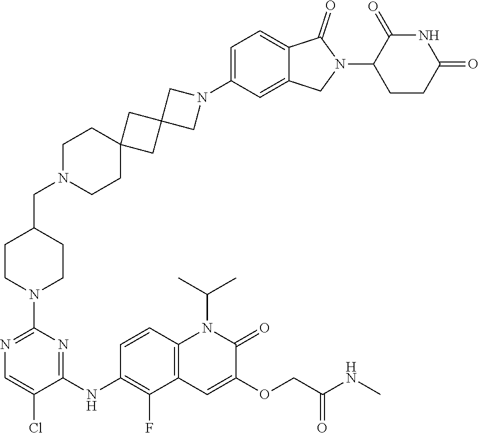 [SMILES: CNC(=O)COc1cc2c(F)c(Nc3nc(N4CCC(CN5CCC6(CC5)CC5(CN(c7ccc8c(c7)CN(C7CCC(=O)NC7=O)C8=O)C5)C6)CC4)ncc3Cl)ccc2n(C(C)C)c1=O] | 2-{[6-({5-chloro-2-[4-({2-[2-(2,6- dioxopiperidin-3-yl)-1-oxo-2,3-dihydro-1H- isoindol-5-yl]-2,9- diazadispiro[3.1.5⁶.1⁴]dodecan-9- yl}methyl)piperidin-1-yl]pyrimidin-4- yl}amino)-5-fluoro-2-oxo-1-(propan-2-yl)- 1,2-dihydroquinolin-3-yl]oxy}-N- methylacetamide |
| 422 | 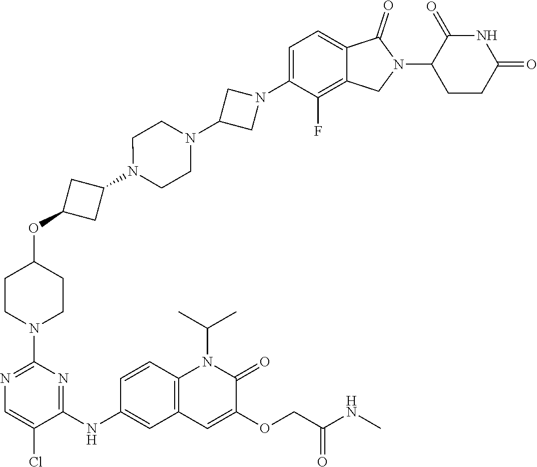 [SMILES: CNC(=O)COc1cc2cc(Nc3nc(N4CCC(O[C@H]5C[C@H](N6CCN(C7CN(c8ccc9c(c8F)CN(C8CCC(=O)NC8=O)C9=O)C7)CC6)C5)CC4)ncc3Cl)ccc2n(C(C)C)c1=O] | 2-({6-[(5-chloro-2-{4-[(1r,3r)-3-(4-{1-[2-(2,6- dioxopiperidin-3-yl)-4-fluoro-1-oxo-2,3- dihydro-1H-isoindol-5-yl]azetidin-3- yl}piperazin-1-yl)cyclobutoxy]piperidin-1- yl}pyrimidin-4-yl)amino]-2-oxo-1-(propan-2- yl)-1,2-dihydroquinolin-3-yl}oxy)-N- methylacetamide |
| 423 | 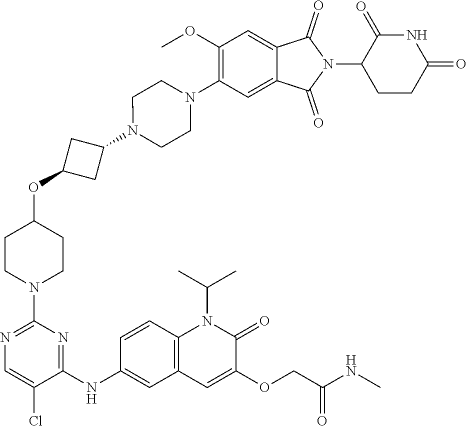 [SMILES: CNC(=O)COc1cc2cc(Nc3nc(N4CCC(O[C@H]5C[C@H](N6CCN(c7cc8c(cc7OC)C(=O)N(C7CCC(=O)NC7=O)C8=O)CC6)C5)CC4)ncc3Cl)ccc2n(C(C)C)c1=O] | 2-({6-[(5-chloro-2-{4-[(1r,3r)-3-{4-[2-(2,6- dioxopiperidin-3-yl)-6-methoxy-1,3-dioxo- 2,3-dihydro-1H-isoindol-5-yl]piperazin-1- yl}cyclobutoxy]piperidin-1-yl}pyrimdiin-4- yl)amino]-2-oxo-1-(propan-2-yl)-1,2- dihydroquinolin-3-yl}oxy)-N- methylacetamide |
| 424 | 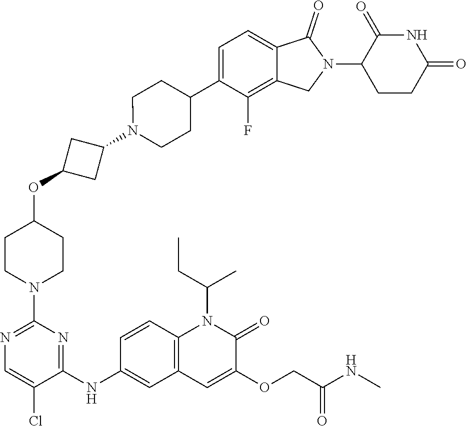 [SMILES: CCC(C)n1c(=O)c(OCC(=O)NC)cc2cc(Nc3nc(N4CCC(O[C@H]5C[C@H](N6CCC(c7ccc8c(c7F)CN(C7CCC(=O)NC7=O)C8=O)CC6)C5)CC4)ncc3Cl)ccc21] | 2-{1-(butan-2-yl)-6-[(5-chloro-2-{4-[(1r,3r)- 3-{4-[2-(2,6-dioxopiperidin-3-yl)-4-fluoro-1- oxo-2,3-dihydro-1H-isoindol-5-yl]piperidin- 1-yl}cyclobutoxy]piperidin-1-yl}pyrimidin-4- yl)amino]-2-oxo-1,2-dihydroquinolin-3- yl]oxy}-N-methylacetamide |
| 425 | 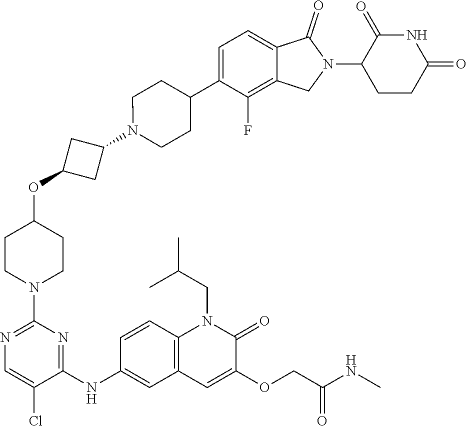 [SMILES: CNC(=O)COc1cc2cc(Nc3nc(N4CCC(O[C@H]5C[C@H](N6CCC(c7ccc8c(c7F)CN(C7CCC(=O)NC7=O)C8=O)CC6)C5)CC4)ncc3Cl)ccc2n(CC(C)C)c1=O] | 2-({6-[(5-chloro-2-{4-[(1r,3r)-3-{4-[2-(2,6- dioxopiperidin-3-yl)-4-fluoro-1-oxo-2,3- dihydro-1H-isoindol-5-yl]piperidin-1- yl}cyclobutoxy]piperidin-1-yl}pyrimidin-4- yl)amino]-1-(2-methylpropyl)-2-oxo-1,2- dihydroquinolin-3-yl}oxy)-N- methylacetamide |
| 426 | 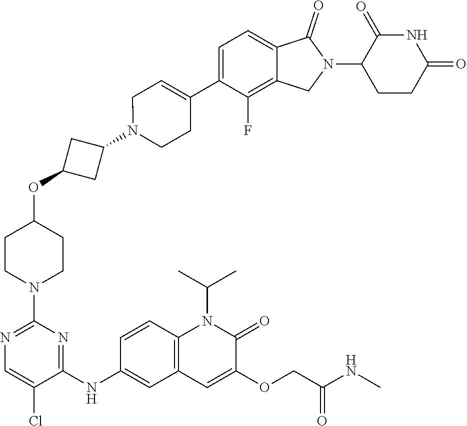 [SMILES: CNC(=O)COc1cc2cc(Nc3nc(N4CCC(O[C@H]5C[C@H](N6CC=C(c7ccc8c(c7F)CN(C7CCC(=O)NC7=O)C8=O)CC6)C5)CC4)ncc3Cl)ccc2n(C(C)C)c1=O] | 2-({6-[(5-chloro-2-{4-[(1r,3r)-3-{4-[2-(2,6- dioxopiperidin-3-yl)-4-fluoro-1-oxo-2,3- dihydro-1H-isoindol-5-yl]-1,2,3,6- tetrahydropyridin-1-yl}cyclobutoxy]piperidin- 1-yl}pyrimidin-4-yl)amino]-2-oxo-1-(propan- 2-yl)-1,2-dihydroquinolin-3-yl}oxy)-N- methylacetamide |
| 427 | 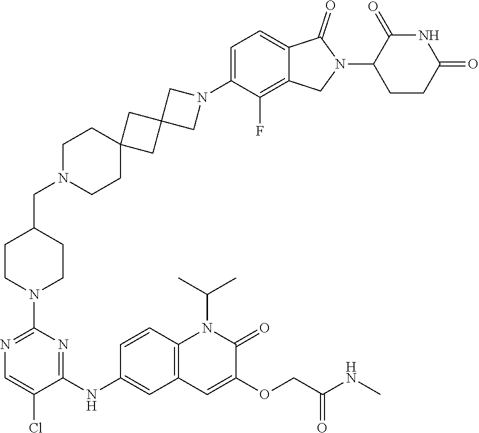 [SMILES: CNC(=O)COc1cc2cc(Nc3nc(N4CCC(CN5CCC6(CC5)CC5(CN(c7ccc8c(c7F)CN(C7CCC(=O)NC7=O)C8=O)C5)C6)CC4)ncc3Cl)ccc2n(C(C)C)c1=O] | |
| 428 | 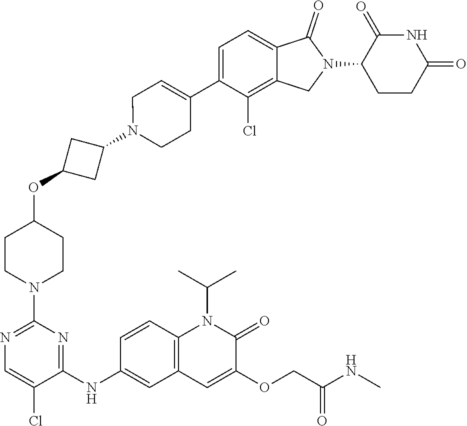 [SMILES: CNC(=O)COc1cc2cc(Nc3nc(N4CCC(O[C@H]5C[C@H](N6CC=C(c7ccc8c(c7Cl)CN([C@H]7CCC(=O)NC7=O)C8=O)CC6)C5)CC4)ncc3Cl)ccc2n(C(C)C)c1=O] | |
| 429 | 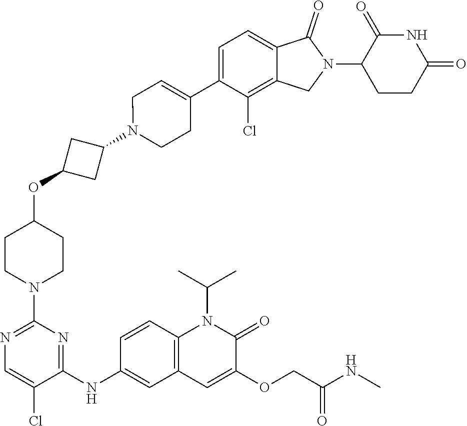 [SMILES: CNC(=O)COc1cc2cc(Nc3nc(N4CCC(O[C@H]5C[C@H](N6CC=C(c7ccc8c(c7Cl)CN(C7CCC(=O)NC7=O)C8=O)CC6)C5)CC4)ncc3Cl)ccc2n(C(C)C)c1=O] | 2-({6-[5-chloro-2-{4-[(1r,3r)-3-{4-[4-chloro- 2-(2,6-dioxopiperidin-3-yl)-1-oxo-2,3- dihydro-1H-isoindol-5-yl]-1,2,3,6- tetrahydropyridin-1-yl}cyclobutoxy]piperidin- 1-yl}pyrimidin-4-yl)amino]-2-oxo-1-(propan- 2-yl)-1,2-dihydroquinolin-3-yl}oxy)-N- methylacetamide |
| 430 | 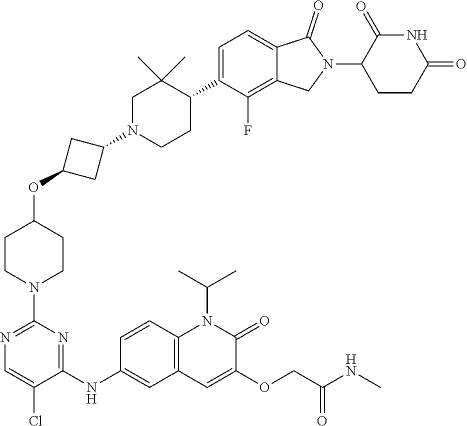 [SMILES: CNC(=O)COc1cc2cc(Nc3nc(N4CCC(O[C@H]5C[C@H](N6CC[C@@H](c7ccc8c(c7F)CN(C7CCC(=O)NC7=O)C8=O)C(C)(C)C6)C5)CC4)ncc3Cl)ccc2n(C(C)C)c1=O] | 2-({6-[(5-chloro-2-{4-[(1r,3r)-3-[(4R)-4-[2- (2,6-dioxopiperidin-3-yl)-4-fluoro-1-oxo-2,3- dihydro-1H-isoindol-5-yl]-3,3- dimethylpiperidin-1- yl]cyclobutoxy]piperidin-1-yl}pyrimidin-4- yl)amino]-2-oxo-1-(propan-2-yl)-1,2- dihydroquinolin-3-yl}oxy)-N- methylacetamide |
| 431 | 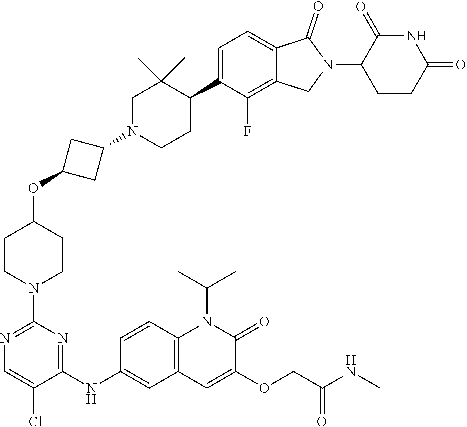 [SMILES: CNC(=O)COc1cc2cc(Nc3nc(N4CCC(O[C@H]5C[C@H](N6CC[C@H](c7ccc8c(c7F)CN(C7CCC(=O)NC7=O)C8=O)C(C)(C)C6)C5)CC4)ncc3Cl)ccc2n(C(C)C)c1=O] | 2-({6-[(5-chloro-2-{4-[(1r,3r)-3-[(4S)-4-[2- (2,6-dioxopiperidin-3-yl)-4-fluoro-1-oxo-2,3- dihydro-1H-isoindol-5-yl]-3,3- dimethylpiperidin-1- yl]cyclobutoxy]piperidin-1-yl}pyrimidin-4- yl)amino]-2-oxo-1-(propan-2-yl)-1,2- dihydroquinolin-3-yl}oxy)-N- methylacetamide |

In some embodiments, the compound of the disclosure is Compound No. 81 (Compound A), Compound No. 163, Compound No. 208, Compound No. 209, Compound No. 211, or a pharmaceutically acceptable salt thereof.

In some embodiments, the compound of the disclosure is:

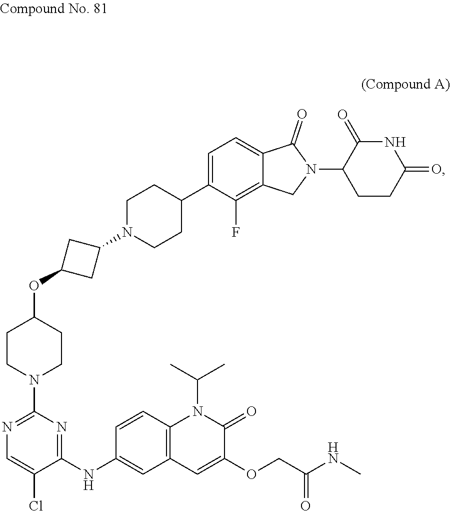 [SMILES: CNC(=O)COc1cc2cc(Nc3nc(N4CCC(O[C@H]5C[C@H](N6CCC(c7ccc8c(c7F)CN(C7CCC(=O)NC7=O)C8=O)CC6)C5)CC4)ncc3Cl)ccc2n(C(C)C)c1=O]

or a pharmaceutically acceptable salt thereof.

In some embodiments, the compound of the disclosure is:

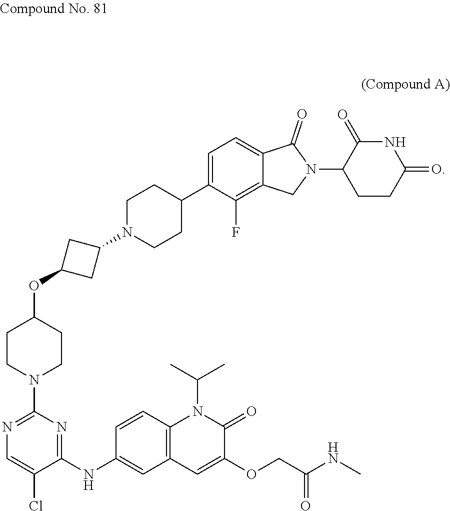 [SMILES: CNC(=O)COc1cc2cc(Nc3nc(N4CCC(O[C@H]5C[C@H](N6CCC(c7ccc8c(c7F)CN(C7CCC(=O)NC7=O)C8=O)CC6)C5)CC4)ncc3Cl)ccc2n(C(C)C)c1=O]

In some embodiments, the compound of the disclosure is Compound No. 163 or a pharmaceutically acceptable salt thereof.

In some embodiments, the compound of the disclosure is Compound No. 163.

In some embodiments, the compound of the disclosure is Compound No. 208 or a pharmaceutically acceptable salt thereof.

In some embodiments, the compound of the disclosure is Compound No. 208.

In some embodiments, the compound of the disclosure is Compound No. 209 or a pharmaceutically acceptable salt thereof.

In some embodiments, the compound of the disclosure is Compound No. 209.

In some embodiments, the compound of the disclosure is Compound No. 211 or a pharmaceutically acceptable salt thereof.

In some embodiments, the compound of the disclosure is Compound No. 211.

A compound of the disclosure may be synthesized using standard synthetic methods and procedures for the preparation of organic molecules and functional group transformations and manipulations, including the use of protective groups, as can be obtained from the relevant scientific literature or from standard reference textbooks in the field in view of this disclosure. Although not limited to any one or several sources, recognized reference textbooks of organic synthesis include: Smith, M. B.; March, J. March's Advanced Organic Chemistry: Reactions, Mechanisms, and Structure, 5^{th} ed.; John Wiley & Sons: New York, 2001; and Greene, T. W.; Wuts, P. G. M. Protective Groups in Organic Synthesis, 3^{rd}; John Wiley & Sons: New York, 1999. The synthetic methods described in U.S. Patent Application Publication No. 2022/0395576 and International Publication No. 2022/221673 are incorporated herein by reference in their entireties.

The term “independently” is used herein to indicate that the variable, which is independently applied, varies independently from application to application.

The term “alkyl” shall mean within its context a linear, branch-chained or cyclic fully saturated hydrocarbon radical or alkyl group, preferably a C₁-C₁₀, more preferably a C₁-C₆, alternatively a C₁-C₃ alkyl group, which may be optionally substituted. Examples of alkyl groups are methyl, ethyl, n-butyl, sec-butyl, n-hexyl, n-heptyl, n-octyl, n-nonyl, n-decyl, isopropyl, 2-methylpropyl, cyclopropyl, cyclopropylmethyl, cyclobutyl, cyclopentyl, cyclopentylethyl, cyclohexylethyl and cyclohexyl, among others. In certain embodiments, the alkyl group is end-capped with a halogen group (At, Br, Cl, F, or I). In certain preferred embodiments, compounds according to the present disclosure which may be used to covalently bind to dehalogenase enzymes. These compounds generally contain a side chain (often linked through a polyethylene glycol group) which terminates in an alkyl group which has a halogen substituent (often chlorine or bromine) on its distal end which results in covalent binding of the compound containing such a moiety to the protein.

The term “alkenyl” refers to linear, branch-chained or cyclic C₂-C₁₀ (preferably C₂-C₆) hydrocarbon radicals containing at least one C═C bond.

The term “alkynyl” refers to linear, branch-chained or cyclic C₂-C₁₀ (preferably C₂-C₆) hydrocarbon radicals containing at least one C≡C bond.

The term “alkylene” when used, refers to a —(CH₂)ₙ— group (n is an integer generally from 0-6), which may be optionally substituted. When substituted, the alkylene group preferably is substituted on one or more of the methylene groups with a C₁-C₆ alkyl group (including a cyclopropyl group or a t-butyl group), but may also be substituted with one or more halo groups, preferably from 1 to 3 halo groups or one or two hydroxyl groups, O—(C₁-C₆ alkyl) groups or amino acid sidechains as otherwise disclosed herein. In certain embodiments, an alkylene group may be substituted with a urethane or alkoxy group (or other group) which is further substituted with a polyethylene glycol chain (of from 1 to 10, preferably 1 to 6, often 1 to 4 ethylene glycol units) to which is substituted (preferably, but not exclusively on the distal end of the polyethylene glycol chain) an alkyl chain substituted with a single halogen group, preferably a chlorine group. In still other embodiments, the alkylene (often, a methylene) group, may be substituted with an amino acid sidechain group such as a sidechain group of a natural or unnatural amino acid, for example, alanine, β-alanine, arginine, asparagine, aspartic acid, cysteine, cystine, glutamic acid, glutamine, glycine, phenylalanine, histidine, isoleucine, lysine, leucine, methionine, proline, serine, threonine, valine, tryptophan or tyrosine.

The term “unsubstituted” shall mean substituted only with hydrogen atoms. A range of carbon atoms which includes C₀ means that carbon is absent and is replaced with H. Thus, a range of carbon atoms which is C₀-C6 includes carbons atoms of 1, 2, 3, 4, 5 and 6 and for C₀, H stands in place of carbon.

The term “substituted” or “optionally substituted” shall mean independently (i.e., where more than substituent occurs, each substituent is independent of another substituent) one or more substituents (independently up to five substituents, preferably up to three substituents, often 1 or 2 substituents on a moiety in a compound according to the present disclosure and may include substituents which themselves may be further substituted) at a carbon (or nitrogen) position anywhere on a molecule within context, and includes as substituents hydroxyl, thiol, carboxyl, cyano (C≡N), nitro (NO₂), halogen (preferably, 1, 2 or 3 halogens, especially on an alkyl, especially a methyl group such as a trifluoromethyl), an alkyl group (preferably, C₁-C₁₀, more preferably, C₁-C₆), aryl (especially phenyl and substituted phenyl for example benzyl or benzoyl), alkoxy group (preferably, C₁-C₆ alkyl or aryl, including phenyl and substituted phenyl), thioether (C₁-C₆ alkyl or aryl), acyl (preferably, C₁-C₆ acyl), ester or thioester (preferably, C₁-C₆ alkyl or aryl) including alkylene ester (such that attachment is on the alkylene group, rather than at the ester function which is preferably substituted with a C₁-C₆ alkyl or aryl group), preferably, C₁-C₆ alkyl or aryl, halogen (preferably, F or Cl), amine (including a five- or six-membered cyclic alkylene amine, further including a C₁-C₆ alkyl amine or a C₁-C₆ dialkyl amine which alkyl groups may be substituted with one or two hydroxyl groups) or an optionally substituted —N(C₀-C₆ alkyl)C(O)(O—C₁-C₆ alkyl) group (which may be optionally substituted with a polyethylene glycol chain to which is further bound an alkyl group containing a single halogen, preferably chlorine substituent), hydrazine, amido, which is preferably substituted with one or two C₁-C₆ alkyl groups (including a carboxamide which is optionally substituted with one or two C₁-C₆ alkyl groups), alkanol (preferably, C₁-C₆ alkyl or aryl), or alkanoic acid (preferably, C₁-C₆ alkyl or aryl). Substituents according to the present disclosure may include, for example —SiR₁R₂R₃ groups where each of R₁ and R₂ is as otherwise described herein and R₃ is H or a C₁-C₆ alkyl group, preferably R₁, R₂, R₃ in this context is a C₁-C₃ alkyl group (including an isopropyl or t-butyl group). Each of the above-described groups may be linked directly to the substituted moiety or alternatively, the substituent may be linked to the substituted moiety (preferably in the case of an aryl or heteroaryl moiety) through an optionally substituted —(CH₂)ₘ— or alternatively an optionally substituted —(OCH₂)ₘ—, —(OCH₂CH₂)ₘ— or —(CH₂CH₂O)ₘ— group, which may be substituted with any one or more of the above-described substituents. Alkylene groups —(CH₂)ₘ— or —(CH₂)ₙ— groups or other chains such as ethylene glycol chains, as identified above, may be substituted anywhere on the chain. Preferred substituents on alkylene groups include halogen or C₁-C₆ (preferably C₁-C₃) alkyl groups, which may be optionally substituted with one or two hydroxyl groups, one or two ether groups (O—C₁-C₆ groups), up to three halo groups (preferably F), or a sidechain of an amino acid as otherwise described herein and optionally substituted amide (preferably carboxamide substituted as described above) or urethane groups (often with one or two C₀-C₆ alkyl substituents, which group(s) may be further substituted). In certain embodiments, the alkylene group (often a single methylene group) is substituted with one or two optionally substituted C₁-C₆ alkyl groups, preferably C₁-C₄ alkyl group, most often methyl or O-methyl groups or a sidechain of an amino acid as otherwise described herein. In the present disclosure, a moiety in a molecule may be optionally substituted with up to five substituents, preferably up to three substituents. Most often, in the present disclosure moieties which are substituted are substituted with one or two substituents.

The term “substituted” (each substituent being independent of any other substituent) shall also mean within its context of use C₁-C₆ alkyl, C₁-C₆ alkoxy, halogen, amido, carboxamido, sulfone, including sulfonamide, keto, carboxy, C₁-C₆ ester (oxyester or carbonylester), C₁-C₆ keto, urethane —O—C(O)—NR₁R₂ or —N(R₁)—C(O)—O—R₁, nitro, cyano and amine (especially including a C₁-C₆ alkylene-NR₁R₂, a mono- or di-C₁-C₆ alkyl substituted amines which may be optionally substituted with one or two hydroxyl groups). Each of these groups contain unless otherwise indicated, within context, between 1 and 6 carbon atoms. In certain embodiments, preferred substituents will include for example, —NH—, —NHC(O)—, —O—, ═O, —(CH₂)ₘ— (here, m and n are in context, 1, 2, 3, 4, 5 or 6), —S—, —S(O)—, SO₂— or —NH—C(O)—NH—, —(CH₂)ₙOH, —(CH₂)ₙSH, —(CH₂)ₙCOOH, C₁-C₆ alkyl, —(CH₂)ₙO—(C₁-C₆ alkyl), —(CH₂)ₙC(O)—(C₁-C₆ alkyl), —(CH₂)ₙOC(O)—(C₁-C₆ alkyl), —(CH₂)ₙC(O)O—(C₁-C₆ alkyl), —(CH₂)ₙNHC(O)—R₁, —(CH₂)ₙC(O)—NR₁R₂, —(OCH₂)ₙOH, —(CH₂O)ₙCOOH, C₁-C₆ alkyl, —(OCH₂)ₙO—(C₁-C₆ alkyl), —(CH₂O)ₙC(O)—(C₁-C₆ alkyl), —(OCH₂)ₙNHC(O)—R₁, —(CH₂O)ₙC(O)—NR₁R₂, —S(O)₂-R_{S}, —S(O)—R_{S} (R_{S} is C₁-C₆ alkyl or a —(CH₂)ₘ—NR₁R₂ group), NO₂, CN or halogen (F, Cl, Br, I, preferably F or Cl), depending on the context of the use of the substituent. R₁ and R₂ are each, within context, H or a C₁-C₆ alkyl group (which may be optionally substituted with one or two hydroxyl groups or up to three halogen groups, preferably fluorine). The term “substituted” shall also mean, within the chemical context of the compound defined and substituent used, an optionally substituted aryl or heteroaryl group or an optionally substituted heterocyclic group as otherwise described herein. Alkylene groups may also be substituted as otherwise disclosed herein, preferably with optionally substituted C₁-C₆ alkyl groups (methyl, ethyl or hydroxymethyl or hydroxyethyl is preferred, thus providing a chiral center), a sidechain of an amino acid group as otherwise described herein, an amido group as described hereinabove, or a urethane group O—C(O)—NR₁R₂ group where R₁ and R₂ are as otherwise described herein, although numerous other groups may also be used as substituents. Various optionally substituted moieties may be substituted with 3 or more substituents, preferably no more than 3 substituents and preferably with 1 or 2 substituents. It is noted that in instances where, in a compound at a particular position of the molecule substitution is required (principally, because of valency), but no substitution is indicated, then that substituent is construed or understood to be H, unless the context of the substitution suggests otherwise.

The term “aryl” or “aromatic”, in context, refers to a substituted (as otherwise described herein) or unsubstituted monovalent aromatic radical having a single ring (e.g., benzene, phenyl, benzyl) or condensed rings (e.g., naphthyl, anthracenyl, phenanthrenyl, etc.) and can be bound to the compound according to the present disclosure at any available stable position on the ring(s) or as otherwise indicated in the chemical structure presented. Other examples of aryl groups, in context, may include heterocyclic aromatic ring systems, “heteroaryl” groups having one or more nitrogen, oxygen, or sulfur atoms in the ring (monocyclic) such as imidazole, furyl, pyrrole, furanyl, thiene, thiazole, pyridine, pyrimidine, pyrazine, triazole, oxazole or fused ring systems such as indole, quinoline, indolizine, azaindolizine, benzofurazan, etc., among others, which may be optionally substituted as described above. Among the heteroaryl groups which may be mentioned include nitrogen-containing heteroaryl groups such as pyrrole, pyridine, pyridone, pyridazine, pyrimidine, pyrazine, pyrazole, imidazole, triazole, triazine, tetrazole, indole, isoindole, indolizine, azaindolizine, purine, indazole, quinoline, dihydroquinoline, tetrahydroquinoline, isoquinoline, dihydroisoquinoline, tetrahydroisoquinoline, quinolizine, phthalazine, naphthyridine, quinoxaline, quinazoline, cinnoline, pteridine, imidazopyridine, imidazotriazine, pyrazinopyridazine, acridine, phenanthridine, carbazole, carbazoline, pyrimidine, phenanthroline, phenacene, oxadiazole, benzimidazole, pyrrolopyridine, pyrrolopyrimidine and pyridopyrimidine; sulfur-containing aromatic heterocycles such as thiophene and benzothiophene; oxygen-containing aromatic heterocycles such as furan, pyran, cyclopentapyran, benzofuran and isobenzofuran; and aromatic heterocycles comprising 2 or more hetero atoms selected from among nitrogen, sulfur and oxygen, such as thiazole, thiadizole, isothiazole, benzoxazole, benzothiazole, benzothiadiazole, phenothiazine, isoxazole, furazan, phenoxazine, pyrazoloxazole, imidazothiazole, thienofuran, furopyrrole, pyridoxazine, furopyridine, furopyrimidine, thienopyrimidine and oxazole, among others, all of which may be optionally substituted.

The term “substituted aryl” refers to an aromatic carbocyclic group comprised of at least one aromatic ring or of multiple condensed rings at least one of which being aromatic, wherein the ring(s) are substituted with one or more substituents. For example, an aryl group can comprise a substituent(s) selected from: —(CH₂)ₙOH, —(CH₂)ₙ—O—(C₁-C₆)alkyl, —(CH₂)ₙ—O—(CH₂)ₙ—(C₁-C₆)alkyl, —(CH₂)ₙ—C(O)(C₀-C₆) alkyl, —(CH₂)ₙ—C(O)O(C₀-C₆)alkyl, —(CH₂)ₙ—OC(O)(C₀-C₆)alkyl, amine, mono- or di-(C₁-C₆ alkyl) amine wherein the alkyl group on the amine is optionally substituted with 1 or 2 hydroxyl groups or up to three halo (preferably F, Cl) groups, OH, COOH, C₁-C₆ alkyl, preferably CH₃, CF₃, OMe, OCF₃, NO₂, or CN group (each of which may be substituted in ortho-, meta- and/or para-positions of the phenyl ring, preferably para-), an optionally substituted phenyl group (the phenyl group itself is preferably connected to a PTM group, including a ULM group, via a linker group), and/or at least one of F, Cl, OH, COOH, CH₃, CF₃, OMe, OCF₃, NO₂, or CN group (in ortho-, meta- and/or para-positions of the phenyl ring, preferably para-), a naphthyl group, which may be optionally substituted, an optionally substituted heteroaryl, preferably an optionally substituted isoxazole including a methylsubstituted isoxazole, an optionally substituted oxazole including a methylsubstituted oxazole, an optionally substituted thiazole including a methyl substituted thiazole, an optionally substituted isothiazole including a methyl substituted isothiazole, an optionally substituted pyrrole including a methylsubstituted pyrrole, an optionally substituted imidazole including a methylimidazole, an optionally substituted benzimidazole or methoxybenzylimidazole, an optionally substituted oximidazole or methyloximidazole, an optionally substituted diazole group, including a methyldiazole group, an optionally substituted triazole group, including a methylsubstituted triazole group, an optionally substituted pyridine group, including a halo- (preferably, F) or methylsubstitutedpyridine group or an oxapyridine group (where the pyridine group is linked to the phenyl group by an oxygen), an optionally substituted furan, an optionally substituted benzofuran, an optionally substituted dihydrobenzofuran, an optionally substituted indole, indolizine or azaindolizine (2, 3, or 4-azaindolizine), an optionally substituted quinoline, and combinations thereof.

“Carboxyl” denotes the group —C(O)OR, where R is hydrogen, alkyl, substituted alkyl, aryl, substituted aryl, heteroaryl or substituted heteroaryl, whereas these generic substituents have meanings which are identical with definitions of the corresponding groups defined herein.

The term “heteroaryl” or “hetaryl” can mean but is in no way limited to an optionally substituted quinoline (which may be attached to the pharmacophore or substituted on any carbon atom within the quinoline ring), an optionally substituted indole (including dihydroindole), an optionally substituted indolizine, an optionally substituted azaindolizine (2, 3 or 4-azaindolizine) an optionally substituted benzimidazole, benzodiazole, benzoxofuran, an optionally substituted imidazole, an optionally substituted isoxazole, an optionally substituted oxazole (preferably methyl substituted), an optionally substituted diazole, an optionally substituted triazole, a tetrazole, an optionally substituted benzofuran, an optionally substituted thiophene, an optionally substituted thiazole (preferably methyl and/or thiol substituted), an optionally substituted isothiazole, an optionally substituted triazole (preferably a 1,2,3-triazole substituted with a methyl group, a triisopropylsilyl group, an optionally substituted —(CH₂)ₘ—O—C₁-C₆ alkyl group or an optionally substituted —(CH₂)ₘ—C(O)—O—C₁-C₆ alkyl group), an optionally substituted pyridine (2-, 3, or 4-pyridine) or a group according to the chemical structure:

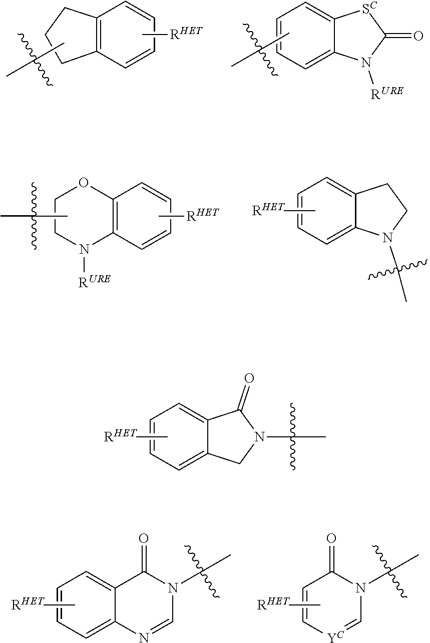

wherein:
S^{c} is CHR^{SS}, NR^{URE}, or O;
R^{HET} is H, CN, NO₂, halo (preferably Cl or F), optionally substituted C₁-C₆ alkyl (preferably substituted with one or two hydroxyl groups or up to three halo groups (e.g. CF₃), optionally substituted O(C₁-C₆ alkyl) (preferably substituted with one or two hydroxyl groups or up to three halo groups) or an optionally substituted acetylenic group —C≡C—Rₐ where Rₐ is H or a C₁-C₆ alkyl group (preferably C₁-C₃ alkyl);
R^{SS} is H, CN, NO₂, halo (preferably F or Cl), optionally substituted C₁-C₆ alkyl (preferably substituted with one or two hydroxyl groups or up to three halo groups), optionally substituted O—(C₁-C₆ alkyl) (preferably substituted with one or two hydroxyl groups or up to three halo groups) or an optionally substituted —C(O)(C₁-C₆ alkyl) (preferably substituted with one or two hydroxyl groups or up to three halo groups);
R^{URE} is H, a C₁-C₆ alkyl (preferably H or C₁-C₃ alkyl) or a —C(O)(C₁-C₆ alkyl), each of which groups is optionally substituted with one or two hydroxyl groups or up to three halogen, preferably fluorine groups, or an optionally substituted heterocycle, for example piperidine, morpholine, pyrrolidine, tetrahydrofuran, tetrahydrothiophene, piperidine, piperazine, each of which is optionally substituted, and
Y^{C} is N or C—R^{YC}, where R^{YC} is H, OH, CN, NO₂, halo (preferably Cl or F), optionally substituted C₁-C₆ alkyl (preferably substituted with one or two hydroxyl groups or up to three halo groups (e.g. CF₃), optionally substituted O(C₁-C₆ alkyl) (preferably substituted with one or two hydroxyl groups or up to three halo groups) or an optionally substituted acetylenic group —C≡C—Rₐ where Rₐ is H or a C₁-C₆ alkyl group (preferably C₁-C₃ alkyl).

The terms “aralkyl” and “heteroarylalkyl” refer to groups that comprise both aryl or, respectively, heteroaryl as well as alkyl and/or heteroalkyl and/or carbocyclic and/or heterocycloalkyl ring systems according to the above definitions.

The term “arylalkyl” as used herein refers to an aryl group as defined above appended to an alkyl group defined above. The arylalkyl group is attached to the parent moiety through an alkyl group wherein the alkyl group is one to six carbon atoms. The aryl group in the arylalkyl group may be substituted as defined above.

The term “heterocycle” refers to a cyclic group which contains at least one heteroatom, e.g., N, O or S, and may be aromatic (heteroaryl) or non-aromatic. Thus, the heteroaryl moieties are subsumed under the definition of heterocycle, depending on the context of its use. Exemplary heteroaryl groups are described hereinabove.

Exemplary heterocyclics include: azetidinyl, benzimidazolyl, 1,4-benzodioxanyl, 1,3-benzodioxolyl, benzoxazolyl, benzothiazolyl, benzothienyl, dihydroimidazolyl, dihydropyranyl, dihydrofuranyl, dioxanyl, dioxolanyl, ethyleneurea, 1,3-dioxolane, 1,3-dioxane, 1,4-dioxane, furyl, homopiperidinyl, imidazolyl, imidazolinyl, imidazolidinyl, indolinyl, indolyl, isoquinolinyl, isothiazolidinyl, isothiazolyl, isoxazolidinyl, isoxazolyl, morpholinyl, naphthyridinyl, oxazolidinyl, oxazolyl, pyridone, 2-pyrrolidone, pyridine, piperazinyl, N-methylpiperazinyl, piperidinyl, phthalimide, succinimide, pyrazinyl, pyrazolinyl, pyridyl, pyrimidinyl, pyrrolidinyl, pyrrolinyl, pyrrolyl, quinolinyl, tetrahydrofuranyl, tetrahydropyranyl, tetrahydroquinoline, thiazolidinyl, thiazolyl, thienyl, tetrahydrothiophene, oxane, oxetanyl, oxathiolanyl, thiane among others.

Heterocyclic groups can be optionally substituted with a member selected from the group consisting of alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxy, carboxyalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl, oxo (═O), and —SO₂-heteroaryl. Such heterocyclic groups can have a single ring or multiple condensed rings. Examples of nitrogen heterocycles and heteroaryls include, but are not limited to, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, morpholino, piperidinyl, tetrahydrofuranyl, and the like as well as N-alkoxy-nitrogen containing heterocycles. The term “heterocyclic” also includes bicyclic groups in which any of the heterocyclic rings is fused to a benzene ring or a cyclohexane ring or another heterocyclic ring (for example, indolyl, quinolyl, isoquinolyl, tetrahydroquinolyl, and the like).

The term “cycloalkyl” can mean but is in no way limited to univalent groups derived from monocyclic or polycyclic alkyl groups or cycloalkanes, as defined herein, e.g., saturated monocyclic hydrocarbon groups having from three to twenty carbon atoms in the ring, including, but not limited to, cyclopropyl, cyclobutyl, cyclopentyl, cyclohexyl, cycloheptyl and the like. The term “substituted cycloalkyl” can mean but is in no way limited to a monocyclic or polycyclic alkyl group and being substituted by one or more substituents, for example, amino, halogen, alkyl, substituted alkyl, carbyloxy, carbylmercapto, aryl, nitro, mercapto or sulfo, whereas these generic substituent groups have meanings which are identical with definitions of the corresponding groups as defined in this legend.

“Heterocycloalkyl” refers to a monocyclic or polycyclic alkyl group in which at least one ring carbon atom of its cyclic structure being replaced with a heteroatom selected from the group consisting of N, O, S or P. “Substituted heterocycloalkyl” refers to a monocyclic or polycyclic alkyl group in which at least one ring carbon atom of its cyclic structure being replaced with a heteroatom selected from the group consisting of N, O, S or P and the group is containing one or more substituents selected from the group consisting of halogen, alkyl, substituted alkyl, carbyloxy, carbylmercapto, aryl, nitro, mercapto or sulfo, whereas these generic substituent group have meanings which are identical with definitions of the corresponding groups as defined in this legend.

The term “hydrocarbyl” shall mean a compound which contains carbon and hydrogen and which may be fully saturated, partially unsaturated or aromatic and includes aryl groups, alkyl groups, alkenyl groups and alkynyl groups.

The term “independently” is used herein to indicate that the variable, which is independently applied, varies independently from application to application.

The term “lower alkyl” refers to methyl, ethyl or propyl

The term “lower alkoxy” refers to methoxy, ethoxy or propoxy.

### Therapeutic Compositions

Therapeutic compositions comprising a compound of the disclosure, or a pharmaceutically acceptable salt thereof, are described herein.

Compounds of the disclosure may be administered in single or divided doses by the oral, parenteral or topical routes. Administration of the active compound may range from continuous (intravenous drip) to several oral administrations per day (for example, Q.I.D.) and may include oral, topical, parenteral, intramuscular, intravenous, sub-cutaneous, transdermal (which may include a penetration enhancement agent), buccal, sublingual and suppository administration, among other routes of administration. Enteric coated oral tablets may also be used to enhance bioavailability of the compounds from an oral route of administration. The most effective dosage form will depend upon the pharmacokinetics of the particular agent chosen as well as the severity of disease in the patient. Compounds of the disclosure may also be administered as sprays, mists, or aerosols for intra-nasal, intra-tracheal or pulmonary administration. The present disclosure therefore also is directed to pharmaceutical compositions comprising an effective amount of a compound of the disclosure or a pharmaceutically acceptable salt thereof as described herein, optionally in combination with a pharmaceutically acceptable carrier, additive or excipient. Compounds of the disclosure may also be administered in immediate release, intermediate release or sustained or controlled release forms. Sustained or controlled release forms are preferably administered orally, but also in suppository and transdermal or other topical forms. Intramuscular injections in liposomal form may also be used to control or sustain the release of compound at an injection site.

The compositions as described herein may be formulated in a conventional manner using one or more pharmaceutically acceptable carriers and may also be administered in controlled-release formulations.

The compositions as described herein may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques.

Sterile injectable forms of the compositions as described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent.

The pharmaceutical compositions as described herein may be orally administered in any orally acceptable dosage form including, but not limited to, capsules, tablets, aqueous suspensions or solutions.

Alternatively, the pharmaceutical compositions as described herein may be administered in the form of suppositories for rectal administration. These can be prepared by mixing the agent with a suitable non-irritating excipient, which is solid at room temperature but liquid at rectal temperature and therefore will melt in the rectum to release the drug.

The amount of compound in a pharmaceutical composition as described herein that may be combined with the carrier materials to produce a single dosage form will vary depending upon the host and disease treated, the particular mode of administration. In some embodiments, the compositions should be formulated to contain between about 0.05 milligram to about 750 milligrams or more, about 1 milligram to about 600 milligrams, or about 10 milligrams to about 500 milligrams of active ingredient, alone or in combination with at least one other compound according to the present disclosure.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease or condition being treated.

A compound of the disclosure be administered by any appropriate route, for example, orally, parenterally, intravenously, intradermally, subcutaneously, or topically, including transdermally, in liquid, cream, gel, or solid form, or by aerosol form.

The active compound is included in the pharmaceutically acceptable carrier or diluent in an amount sufficient to deliver to a patient an effective amount for the desired indication, without causing serious toxic effects in the patient treated. A preferred dose of the active compound for all of the herein-mentioned conditions is in the range from about 10 ng/kg to about 300 mg/kg, about 0.1 to about 100 mg/kg per day, or about 0.5 to about 25 mg per kilogram body weight of the recipient/patient per day. A typical topical dosage will range from 0.01-5% wt/wt in a suitable carrier.

The compound is conveniently administered in any suitable unit dosage form, including but not limited to one containing less than 1 mg, 1 mg to 3000 mg, 5 to 500 mg of active ingredient per unit dosage form.

The active ingredient is preferably administered to achieve peak plasma concentrations of the active compound of about 0.00001-30 mM, or about 0.1-30 PM. This may be achieved, for example, by the intravenous injection of a solution or formulation of the active ingredient, optionally in saline, or an aqueous medium or administered as a bolus of the active ingredient. Oral administration is also appropriate to generate effective plasma concentrations of active agent.

The concentration of active compound in the drug composition will depend on absorption, distribution, inactivation, and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient may be administered at once, or may be divided into a number of smaller doses to be administered at varying intervals of time.

Oral compositions will generally include an inert diluent or an edible carrier. They may be enclosed in gelatin capsules or compressed into tablets. For the purpose of oral therapeutic administration, the active compound or its prodrug derivative can be incorporated with excipients and used in the form of tablets, troches, or capsules. Pharmaceutically compatible binding agents, and/or adjuvant materials can be included as part of the composition.

The active compound or pharmaceutically acceptable salt thereof can be administered as a component of an elixir, suspension, syrup, wafer, chewing gum or the like.

Solutions or suspensions used for parenteral, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parental preparation can be enclosed in ampoules, disposable syringes or multiple dose vials made of glass or plastic.

In one embodiment, the active compounds are prepared with carriers that will protect the compound against rapid elimination from the body, such as a controlled release formulation, including implants and microencapsulated delivery systems. Methods for preparation of such formulations will be apparent to those skilled in the art.

Liposomal suspensions may also be pharmaceutically acceptable carriers. These may be prepared according to methods known to those skilled in the art, for example, as described in U.S. Pat. No. 4,522,811 (which is incorporated herein by reference in its entirety). For example, liposome formulations may be prepared by dissolving appropriate lipid(s) in an inorganic solvent that is then evaporated, leaving behind a thin film of dried lipid on the surface of the container. An aqueous solution of the active compound are then introduced into the container. The container is then swirled by hand to free lipid material from the sides of the container and to disperse lipid aggregates, thereby forming the liposomal suspension.

The active compound or pharmaceutically acceptable salt thereof can also be mixed with other active materials that do not impair the desired action, or with materials that supplement the desired action, such as anti-cancer agents, as described herein among others. In certain preferred aspects of the disclosure, one or more compounds according to the present disclosure are coadministered with another bioactive agent, such as an anti-cancer agent or a wound-healing agent, including an antibiotic, as otherwise described herein.

### Therapeutic Methods

In some embodiments, the present disclosure provides a method of treating or ameliorating a disease or disorder disclosed herein in a subject in need thereof, comprising administering to the subject an effective amount of a compound of the present disclosure or a pharmaceutically acceptable salt thereof. In some embodiments, the compound is selected from the group listed in Table 1, or a pharmaceutically acceptable salt thereof. In some embodiments, the compound is selected from Compound No. 81 (Compound A), Compound No. 163, Compound No. 208, Compound No. 209, Compound No. 211, or a pharmaceutically acceptable salt thereof.

In some embodiments, the compound is

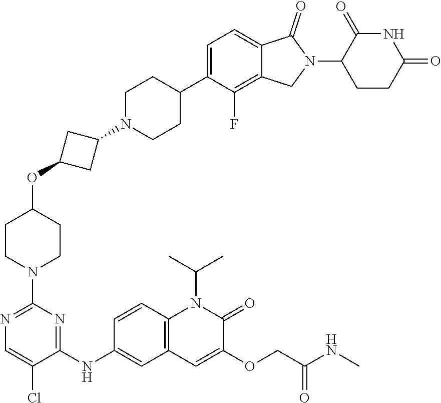 [SMILES: CNC(=O)COc1cc2cc(Nc3nc(N4CCC(O[C@H]5C[C@H](N6CCC(c7ccc8c(c7F)CN(C7CCC(=O)NC7=O)C8=O)CC6)C5)CC4)ncc3Cl)ccc2n(C(C)C)c1=O]

or a pharmaceutically acceptable salt thereof. In some embodiments, the compound is Compound A.

In some embodiments, the present disclosure provides a method of treating or ameliorating a disease or disorder disclosed herein in a subject in need thereof, comprising administering to the subject an effective amount of a compound of the present disclosure. In some embodiments, the compound is

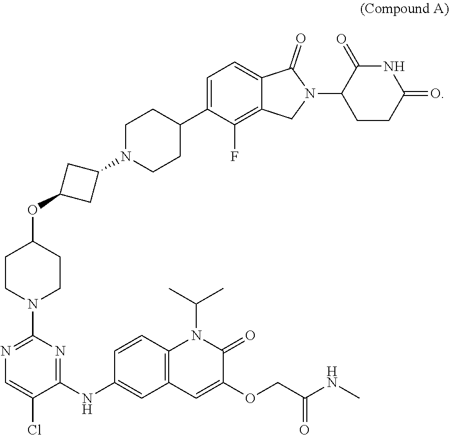 [SMILES: CNC(=O)COc1cc2cc(Nc3nc(N4CCC(O[C@H]5C[C@H](N6CCC(c7ccc8c(c7F)CN(C7CCC(=O)NC7=O)C8=O)CC6)C5)CC4)ncc3Cl)ccc2n(C(C)C)c1=O]

In some embodiments, the present disclosure provides a method of treating or ameliorating a disease or disorder disclosed herein in a subject in need thereof, comprising administering to the subject an effective amount of a compound of the present disclosure. In some embodiments, the compound is

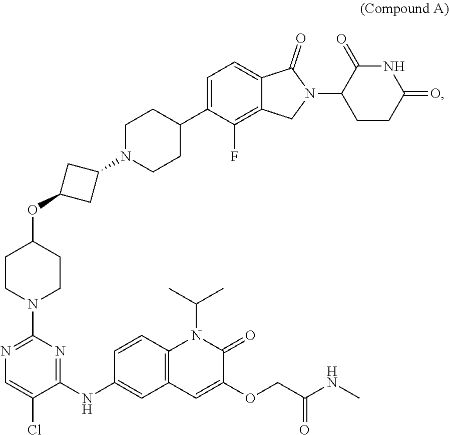 [SMILES: CNC(=O)COc1cc2cc(Nc3nc(N4CCC(O[C@H]5C[C@H](N6CCC(c7ccc8c(c7F)CN(C7CCC(=O)NC7=O)C8=O)CC6)C5)CC4)ncc3Cl)ccc2n(C(C)C)c1=O]

or a pharmaceutically acceptable salt thereof.

The terms “treat”, “treating”, and “treatment”, etc., as used herein, refer to any action providing a benefit to a patient for which the present compounds may be administered, including the treatment of any disease state or condition which is modulated through the protein to which the present compounds bind. Disease states or conditions, including cancer, which may be treated using compounds according to the present disclosure are set forth hereinabove.

In some embodiments, the disease or disorder is associated with aberrant BCL6 expression and or activity.

In some embodiments, the disease or disorder is a cancer associated with aberrant BCL6 expression and or activity.

In some embodiments, the disease or disorder is associated with BCL6 accumulation and aggregation.

In some embodiments, the disease or disorder is a cancer associated with BCL6 accumulation and aggregation.

In some embodiments, the disease or disorder is cancer.

In some embodiments, the cancer is angioimmunoblastic T-cell lymphoma, diffuse large B-cell lymphoma (DLBCL), mature B-cell neoplasm, transformed follicular lymphoma, high grade B-cell lymphoma, germinal center B-cell (GCB) DLBCL, activated B-cell (ABC) DLBCL, non-Hodgkin lymphoma not otherwise specified, or solid tumors.

In some embodiments, the cancer is angioimmunoblastic T-cell lymphoma, diffuse large B-cell lymphoma (DLBCL), transformed follicular lymphoma, high grade B-cell lymphoma, germinal center B-cell (GCB) DLBCL, activated B-cell (ABC) DLBCL, and non-Hodgkin lymphoma not otherwise specified, or solid tumors.

As used herein, “angioimmunoblastic T-cell lymphoma” is also known as “AITL” or “Nodal T-follicular helper (TFH) cell lymphoma, angioimmunoblastic-type.”

In some embodiments, the cancer is angioimmunoblastic T-cell lymphoma, diffuse large B-cell lymphoma (DLBCL), high grade B-cell lymphoma, germinal center B-cell (GCB) DLBCL, activated B-cell (ABC) DLBCL, non-Hodgkin lymphoma not otherwise specified, or solid tumors.

In some embodiments, the cancer is angioimmunoblastic T-cell lymphoma, diffuse large B-cell lymphoma (DLBCL), transformed follicular lymphoma, high grade B-cell lymphoma, non-Hodgkin lymphoma not otherwise specified, or solid tumors.

In some embodiments, the cancer is angioimmunoblastic T-cell lymphoma, diffuse large B-cell lymphoma (DLBCL), or solid tumors.

In some embodiments, the cancer is angioimmunoblastic T-cell lymphoma.

In some embodiments, the cancer is diffuse large B-cell lymphoma (DLBCL).

In some embodiments, the cancer is transformed follicular lymphoma.

In some embodiments, the cancer is high grade B-cell lymphoma.

In some embodiments, the cancer is diffuse large B-cell lymphoma (DLBCL), wherein the diffuse large B-cell lymphoma (DLBCL) is selected from germinal center B-cell (GCB) DLBCL and activated B-cell (ABC) DLBCL

In some embodiments, the cancer is germinal center B-cell (GCB) DLBCL.

In some embodiments, the cancer is activated B-cell (ABC) DLBCL.

In some embodiments, the cancer is non-Hodgkin lymphoma not otherwise specified.

In some embodiments, the cancer is mature B-cell neoplasm.

In some embodiments, the cancer is solid tumors.

In some embodiments, the cancer is solid tumors, wherein the solid tumors are selected from breast cancer, lung cancer, ovarian cancer, neuroblastoma, and glioblastoma.

In some embodiments, the cancer is breast cancer.

In some embodiments, the cancer is lung cancer.

In some embodiments, the cancer is ovarian cancer.

In some embodiments, the cancer is neuroblastoma.

In some embodiments, the cancer is glioblastoma.

The term “neoplasia” or “cancer” is used throughout the specification to refer to the pathological process that results in the formation and growth of a cancerous or malignant neoplasm, i.e., abnormal tissue that grows by cellular proliferation, often more rapidly than normal and continues to grow after the stimuli that initiated the new growth cease. Malignant neoplasms show partial or complete lack of structural organization and functional coordination with the normal tissue and most invade surrounding tissues, metastasize to several sites, and are likely to recur after attempted removal and to cause the death of the patient unless adequately treated. As used herein, the term neoplasia is used to describe all cancerous disease states and embraces or encompasses the pathological process associated with malignant hematogenous, ascitic and solid tumors. Exemplary cancers which may be treated by the present compounds either alone or in combination with at least one additional anti-cancer agent include diffuse large B-cell lymphoma (DLBCL), angioimmunoblastic T-cell lymphoma, mature B-cell neoplasm, transformed follicular lymphoma, high grade B-cell lymphoma, germinal center B-cell (GCB) DLBCL, activated B-cell (ABC) DLBCL, non-Hodgkin lymphoma not otherwise specified, or solid tumors, including, but not limited to, breast, lung and ovarian cancers, and neuroblastoma and glioblastoma.

In some embodiments, the present disclosure provides a method of treating or ameliorating diffuse large B-cell lymphoma (DLBCL) in a subject in need thereof, comprising administering to the subject an effective amount of a compound of the present disclosure. In some embodiments, the compound is selected from the group listed in Table 1, or a pharmaceutically acceptable salt thereof. In some embodiments, the compound is

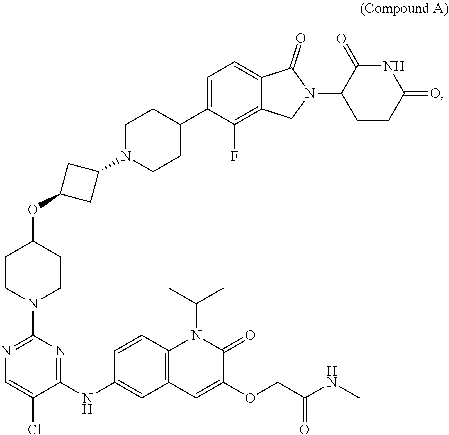 [SMILES: CNC(=O)COc1cc2cc(Nc3nc(N4CCC(O[C@H]5C[C@H](N6CCC(c7ccc8c(c7F)CN(C7CCC(=O)NC7=O)C8=O)CC6)C5)CC4)ncc3Cl)ccc2n(C(C)C)c1=O]

or a pharmaceutically acceptable salt thereof. In some embodiments, the compound is Compound A. In some embodiments, the compound of the disclosure is Compound No. 163 or a pharmaceutically acceptable salt thereof. In some embodiments, the compound of the disclosure is Compound No. 163. In some embodiments, the compound of the disclosure is Compound No. 208 or a pharmaceutically acceptable salt thereof. In some embodiments, the compound of the disclosure is Compound No. 208. In some embodiments, the compound of the disclosure is Compound No. 209 or a pharmaceutically acceptable salt thereof. In some embodiments, the compound of the disclosure is Compound No. 209. In some embodiments, the compound of the disclosure is Compound No. 211 or a pharmaceutically acceptable salt thereof. In some embodiments, the compound of the disclosure is Compound No. 211.

### EXAMPLES

### Example 1. Compound a Degrades BCL6 Protein in GCB and ABC DLBCL Cell Lines

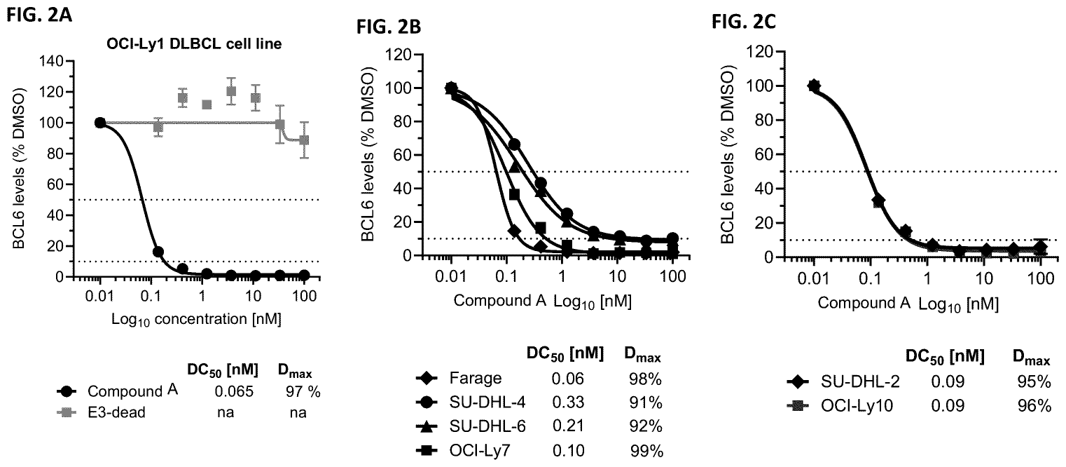

Degradation of BCL6 protein in DLBCL cell lines were measured by ELISA. OCI-Ly1 cell line was treated with Compound A and its E3-binding deficient analogue for 24-hours (FIG. 2A). GCB DLBCL cell lines Farage, SU-DHL-4, SU-DHL-6 and OCI-Ly7 (FIG. 2B) and ABC DLBCL cell lines SU-DHL-2 and OCI-Ly10 (FIG. 2C) were treated for 24 hours with Compound A. FIG. 2A showed that Compound A treatment demonstrated potent BCL6 protein degradation in OCI-Ly1 cell line compared to its E3-binding deficient analogue. FIG. 2B and FIG. 2C showed that Compound A treatment demonstrated potent BCL6 protein degradation in GCB and ABC DLBCL cell lines.

### Example 2. Compound a Inhibits the Proliferation of DLBCL Cell Lines

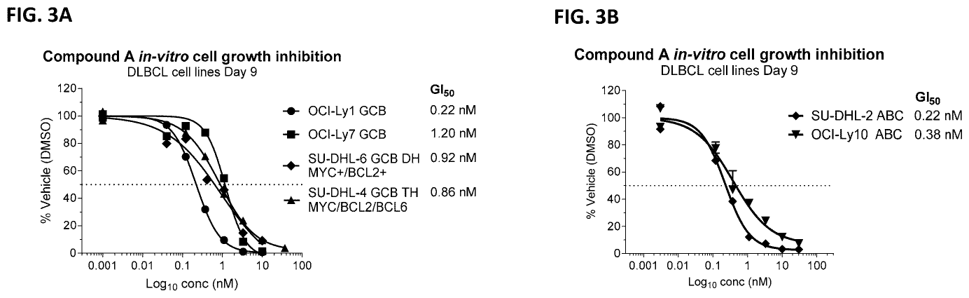

Antiproliferative effects of Compound A on DLBCL-derived cell lines were tested. Nine-day cell growth inhibition assays were conducted using GCB lines OCI-Ly1, OCI-Ly7, SU-DHL-4 and SU-DHL-6 (FIG. 3A), and ABC lines SU-DHL-2 and OCI-Ly10 (FIG. 3B) dosed with a 7-point 3-fold serial dilution of Compound A at a top dose of 30 nM. On days 3 and 6-7, samples were split to new 48-well plates for re-treatment, and 20% to a 96-well plate to measure cell viability using CellTiter-Glo (CTG). Split cells in the 48-well plates were retreated and cultured out to day 9, when 20% of each sample was analyzed by CTG. Dose response was plotted as a percentage of vehicle (DMSO) control (FIG. 3A and FIG. 3B). Graphs are representative of at least two independent proliferation assays per cell line. Each Compound A treatment demonstrated a significant decrease on DLBCL cells growth, the effect was concentration dependent.

### Example 3. Compound a Inhibits the Tumor Growth of DLBCL Cell Line Derived Xenograft Model OCI-Ly1

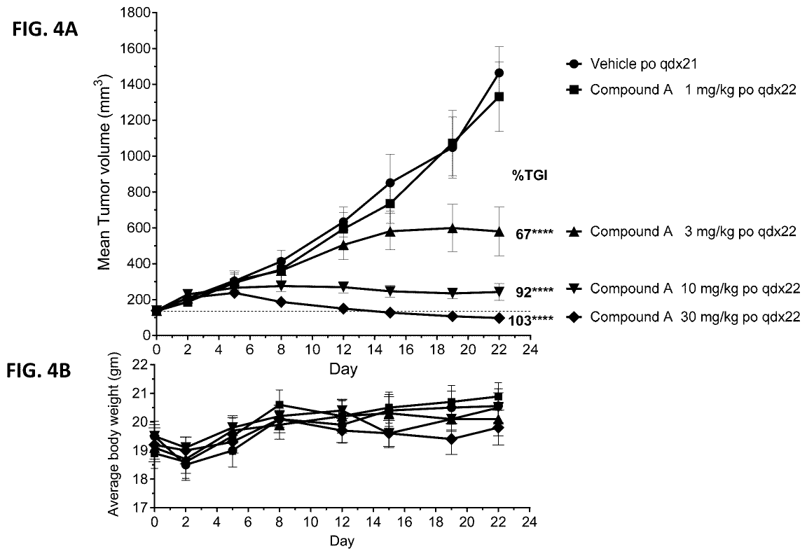

Mice bearing subcutaneous tumors were orally (po) administered vehicle or Compound A at 1, 3, 10, or 30 mg/kg daily for 22 days (qd×22). Dosing began with tumors at an average of 140 mm³. FIG. 4A shows average OCI-Ly1 tumor growth. FIG. 4B shows average body weights of treated mice. Statistical analyses were performed using 2-way ANOVA: p<0.0001 (****). Error bars represent standard error of the mean (±SEM). The data indicate that the treatment with compound A inhibits the tumor growth of DLBCL cell line derived xenograft model OCI-Ly1, the effect was dosing dependent.

### Example 4. Tumor Lysate BCL6 Protein Levels in OCI-Ly1 Cell Line Xenograft Tumor Tissue Following a Treatment Time-Course with Compound A

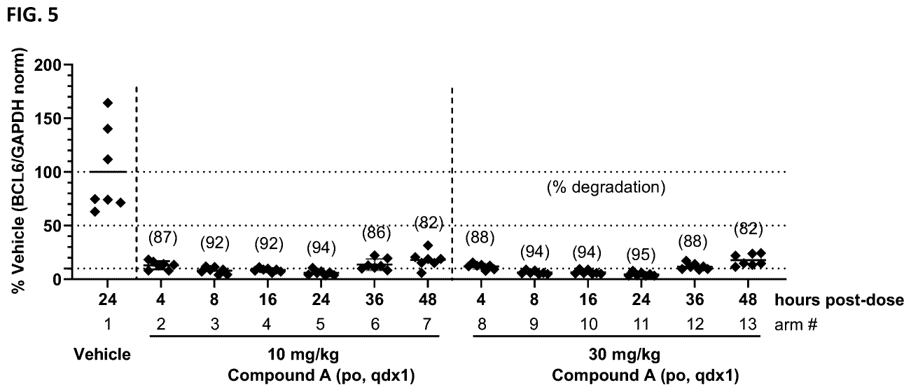

Tumor lysate BCL6 protein levels in OCI-Ly1 cell line xenograft tumor tissue was measured following a treatment time-course with Compound A. A single dose of Compound A was orally administered in a vehicle of 40% hydroxypropyl-b-cyclodextrin in pH 3.0 citrate buffer when tumors reached 200-400 mm³. Tumors were harvested at the indicated timepoints. Tumor tissue lysates were analyzed using western immunoblotting procedures and densitometry. BCL6 levels are normalized to a GAPDH loading control and presented as a percentage (%) of the average of BCL6 levels in the vehicle control group. Percent BCL6 degradation (%) relative to vehicle is shown above each arm. Cells (1×10⁷ cells in 50% matrigel+50% RMPI-1640 (phenol red-free)/100 μl/mouse) were implanted on the right flank subcutaneously. FIG. 5 showed that Compound A treatment degraded BCL6 protein rapidly and kept them down for up to 36 hours in the OCI-Ly1 model.

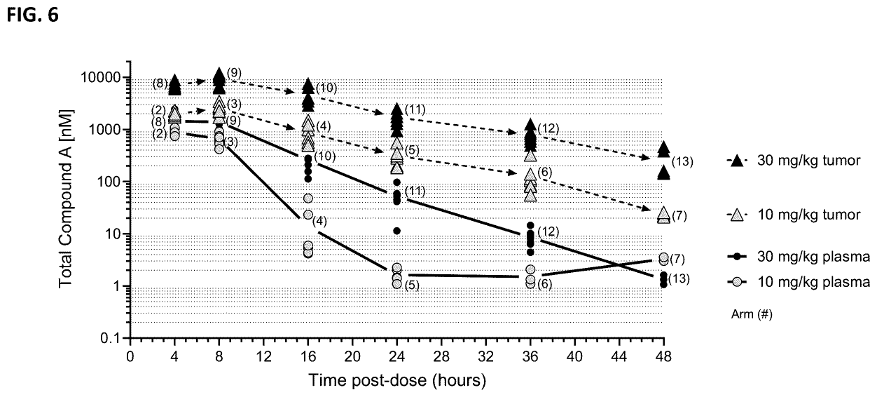

Additionally, FIG. 6 showed the total Compound A plasma and tumor levels following a treatment time-course with Compound A. Curves represented average drug levels within each dose group for each tissue type over time. Individual data points were shown to demonstrate spread of the data within each group of seven mice per condition assayed. Compound A achieved maximum concentration (Cₘₐₓ) at 4-hours in plasma and at 8-hours in tumor tissues.

### Example 5. Compound a Inhibits the Tumor Growth of DLBCL Cell Line Derived Xenograft Model OCI-Ly1

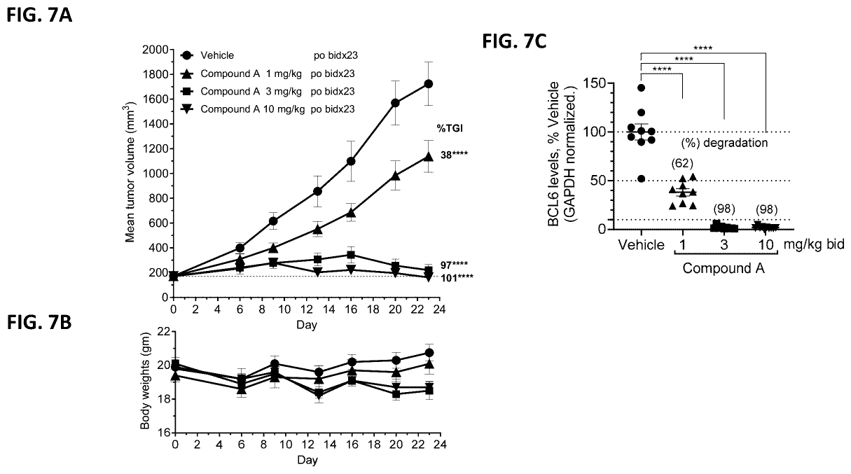

Mice bearing subcutaneous tumors were orally (po) administered vehicle or Compound A at 1, 3, or 10 mg/kg bi-daily for 23 days (bid×23). Dosing began with tumors at an average of 170 mm³. FIG. 7A and FIG. 7B indicate that the treatment with compound A inhibits the tumor growth of DLBCL cell line derived xenograft model OCI-Ly1, the effect was dosing dependent. FIG. 7C depicts tumor lysate BCL6 protein levels 16-hours post-last dose analyzed by western blotting. Individual tumor BCL6 levels are shown in a scatter plot, mean is represented by the bar, determined by densitometry, normalized to GAPDH loading controls, and shown as a percentage of vehicle. Percent BCL6 degradation (%) relative to vehicle is shown above each arm. Statistical analyses were performed using 2-way ANOVA: p<0.0001 (****). Error bars represent standard error of the mean (±SEM). FIG. 7C showed that tumor growth inhibition by Compound A treatment was associated with a dose-dependent degradation of BCL6 protein in the OCI-Ly1 model.

### Example 6. Compound a Inhibits the Tumor Growth of DLBCL Cell Line Derived Xenograft Model OCI-Ly7

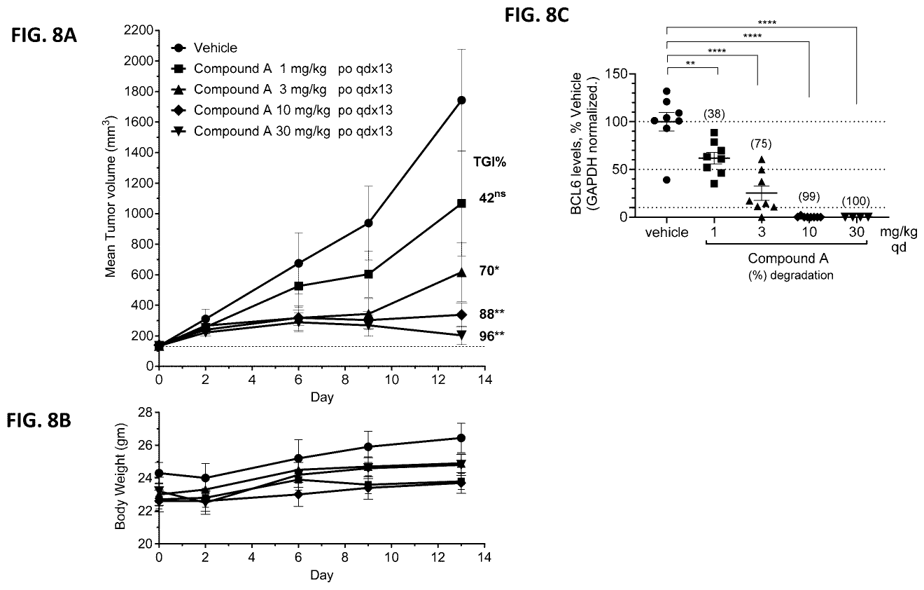

Mice bearing subcutaneous tumors were orally (po) administered vehicle or Compound A at 1, 3, 10, or 30 mg/kg daily for 13 days (qd×13). Dosing began with tumors at an average of 130 mm³. FIG. 8A and FIG. 8B indicate that the treatment with compound A inhibits the tumor growth of DLBCL cell line derived xenograft model OCI-Ly7, the effect was dosing dependent. FIG. 8C depicts tumor lysate BCL6 protein levels 16-hours post-last dose analyzed by western blotting. Individual tumor BCL6 levels are shown in a scatter plot, mean is represented by the bar, determined by densitometry, normalized to GAPDH loading controls, and shown as a percentage of vehicle. Percent BCL6 degradation (%) relative to vehicle is shown above each arm. Statistical analyses were performed using 2-way ANOVA: p<0.0001 (****). Error bars represent standard error of the mean (±SEM). FIG. 8C showed that tumor growth inhibition by Compound A treatment was associated with a dose-dependent degradation of BCL6 protein in the OCI-Ly7 model.

### Example 7. Compound a Inhibits the Tumor Growth of DLBCL Cell Line Derived Xenograft Model SU-DHL-2

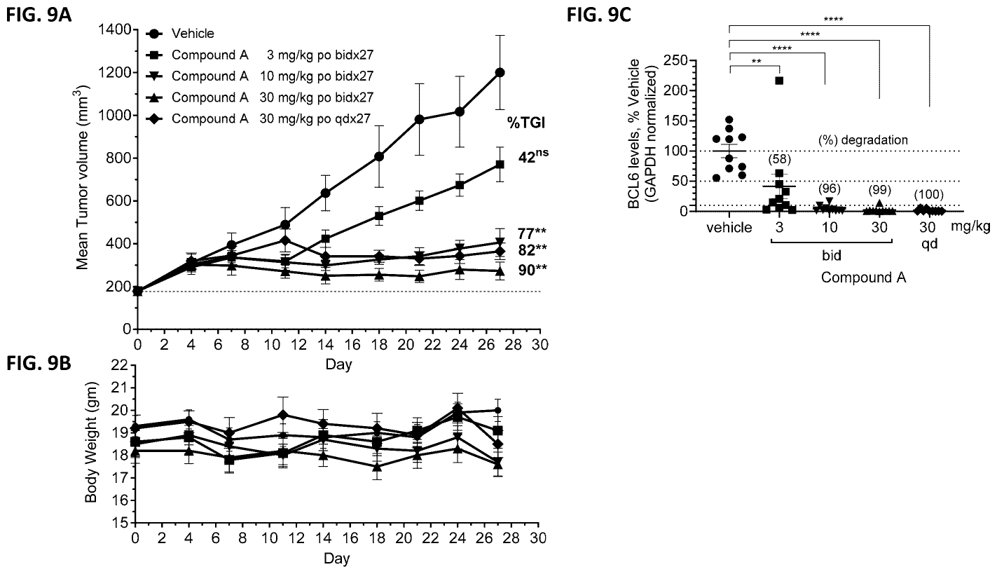

Mice bearing subcutaneous tumors were orally (po) administered vehicle or Compound A at 3, 10, or 30 mg/kg bi-daily for 27 days (bid×27) or 30 mg/kg daily for 27 days (qd×27). Dosing began with tumors at an average of 180 mm³. FIG. 9A and FIG. 9B indicate that the treatment with compound A inhibits the tumor growth of DLBCL cell line derived xenograft model SU-DHL-2, the effect was dosing dependent. FIG. 9C depicts tumor lysate BCL6 protein levels 16-hours post-last dose analyzed by western blotting. Individual tumor BCL6 levels are shown in a scatter plot, mean is represented by the bar, determined by densitometry, normalized to GAPDH loading controls, and shown as a percentage of vehicle. Percent BCL6 degradation (%) relative to vehicle is shown above each arm. Statistical analyses were performed using 2-way ANOVA: p<0.0001 (****). Error bars represent standard error of the mean (±SEM). FIG. 8C showed that tumor growth inhibition by Compound A treatment was associated with a dose-dependent degradation of BCL6 protein in the SU-DHL-2 model.

### Example 8. Compound a Inhibits the Tumor Growth of DLBCL Cell Line Derived Xenograft Model OCI-Ly10

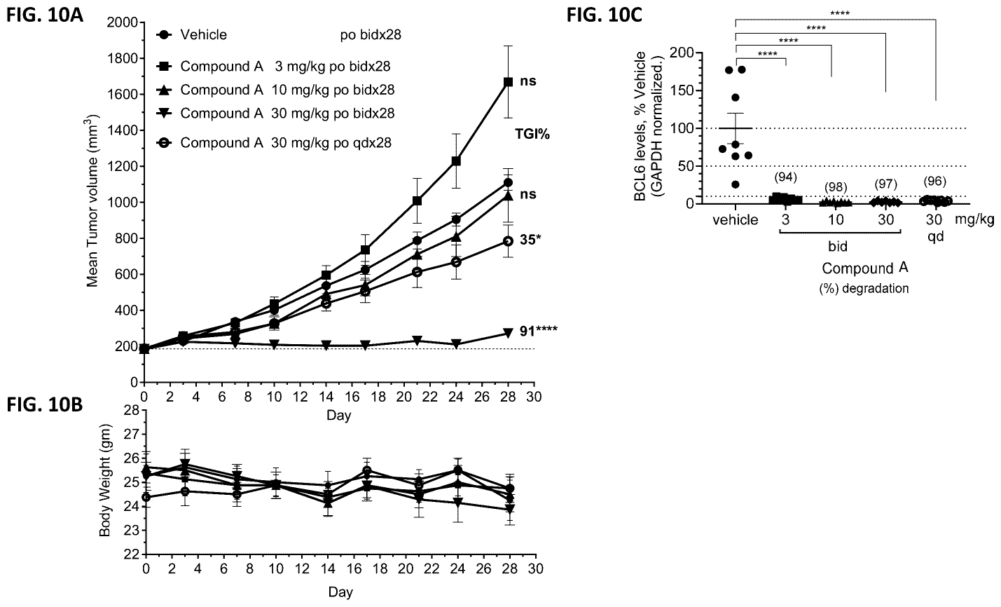

Mice bearing subcutaneous tumors were orally (po) administered vehicle or Compound A at 3, 10, or 30 mg/kg bi-daily for 28 days (bid×28) or 30 mg/kg daily for 28 days (qd×28). Dosing began with tumors at an average of 190 mm³. FIG. 10A and FIG. 10B indicate that the treatment with compound A inhibits the tumor growth of DLBCL cell line derived xenograft model OCI-Ly10, the effect was dosing dependent. FIG. 10C depicts tumor lysate BCL6 protein levels 16-hours post-last dose analyzed by western blotting. Individual tumor BCL6 levels are shown in a scatter plot, mean is represented by the bar, determined by densitometry, normalized to GAPDH loading controls, and shown as a percentage of vehicle. Percent BCL6 degradation (%) relative to vehicle is shown above each arm. Statistical analyses were performed using 2-way ANOVA: p<0.05 (*), p<0.0001 (****). Error bars represent standard error of the mean (±SEM). FIG. 10C showed that Compound A treatment at all dose levels degraded BCL6 protein in the OCI-Ly10 model.

### Example 9: TGI Table

TABLE 2
Compounds of the invention that display >75% TGI
in a BCL6 sensitive tumor xenograft model (OCI-Ly1).

| Compound No. | Ly-1 TGI @ 10 mg/kg (BID) |
|---|---|
| Compound No. 81 (Compound A) | 100% |
| Compound No. 163 | 95% |
| Compound No. 208 | 77% |
| Compound No. 209 | 104% |
| Compound No. 211 | 87% |

### Example 10: Change in Mean Tumor Volume for PDX Mouse Models of DLBCL, Burkitt's Lymphoma, and NHL not Otherwise Specified, Treated with Compound A or Vehicle

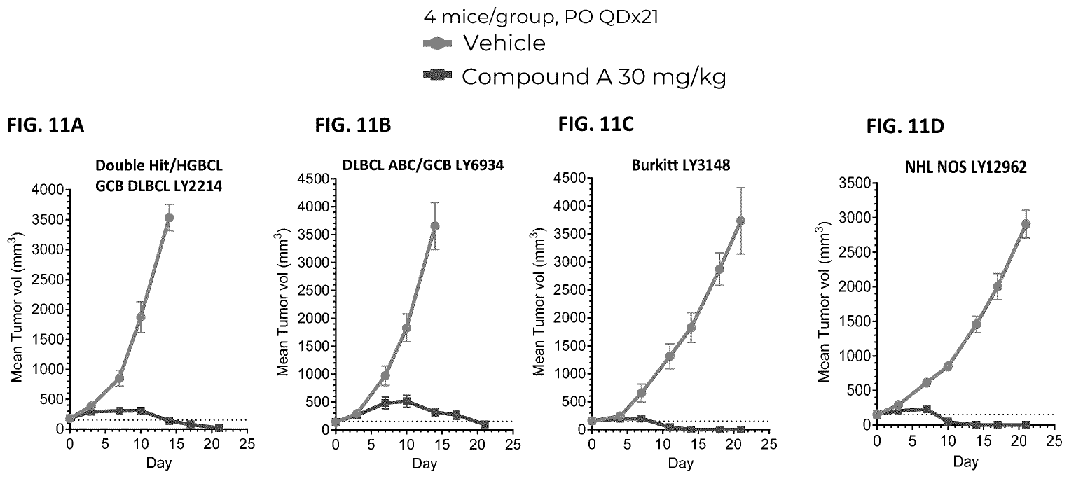

Compound A orally dosed at 30 mg/kg induces in-vivo tumor regressions in patient derived xenograft (PDX) models of DLBCL, Burkitt's lymphoma and NHL not otherwise specified (NOS). The results are shown in FIGS. 11A-11D. The DLBCL models representing two germinal center derived B-cell lymphomas of 1) High-grade B-cell lymphoma (HGBCL, LY2214) and 2) ABC/GCB (LY6934) subtypes, show sensitivity to Compound A resulting in tumor regression compared to vehicle dosed controls. Compound A also induces tumor regressions in models of Burkitt's lymphoma (LY3148) and NHL NOS (LY12962). HuPrime® Lymphoma Xenograft Models in NOD/SCID or BALB/c Nude Mice, 4/group, were dosed orally (per os, PO) daily for 21 days (QD×21) and tumor measurements were acquired twice per week. No significant body weight loss was observed (not shown). Data points represent the mean per group and error bars the standard error of the mean.

Example 11: Preparation of 2-[[6-[[5-chloro-2-[4-[3-[4-[2-(2,6-dioxo-3-piperidyl)-4-methoxy-1-oxo-isoindolin-5-yl]-1-piperidyl]cyclobutoxy]-1-piperidyl]pyrimidin-4-yl]amino]-1-isopropyl-2-oxo-3-quinolyl]oxy]-N-methyl-acetamide (Compound 72)

### Step 1: Preparation of methyl 4-bromo-3-hydroxy-2-methyl-benzoate

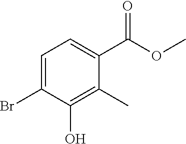

To a solution of 2-methylpropan-2-amine (440 mg, 6.02 mmol, 0.6 mL, 1 eq) in dichloromethane (40 mL) at −70° C. was added a solution of bromine (961 mg, 6.02 mmol, 0.3 mL, 1 eq) in dichloromethane (2 mL) drop wise and the mixture was stirred at −70° C. for 1 hour. A solution of methyl 3-hydroxy-2-methyl-benzoate (1 g, 6.02 mmol, 1 eq) in dichloromethane (2 mL) was then added drop wise and the resulting mixture allowed to warm to 25° C. and stirred for 11 h. The reaction mixture was diluted with water (200 mL) and extracted with dichloromethane (200 mL×2). The combined organic phase was dried with anhydrous sodium sulfate, filtered and concentrated in vacuum. The residue was purified by silica gel chromatography (Petroleum ether/Ethyl acetate=1/0 to 150/1). Compound methyl 4-bromo-3-hydroxy-2-methyl-benzoate (780 mg, 3.18 mmol, 52% yield) was obtained as a white solid. ¹H NMR (400 MHz, DMSO-d6) δ: 9.38 (s, 1H), 7.46 (d, J=8.4 Hz, 1H), 7.18 (d, J=8.4 Hz, 1H), 3.81 (s, 3H), 2.38 (s, 3H). MS (ESI) m/z: 246.9 [M+1]⁺

### Step 2: Preparation of methyl 4-bromo-3-methoxy-2-methyl-benzoate

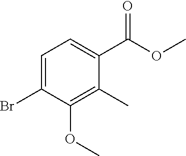

To a solution of methyl 4-bromo-3-hydroxy-2-methyl-benzoate (780 mg, 3.18 mmol, 1 eq) in acetonitrile (6 mL) was added potassium carbonate (527 mg, 3.82 mmol, 1.2 eq) and iodomethane (1.36 g, 9.55 mmol, 0.5 mL, 3 eq). The mixture was stirred at 50° C. for 5 hr. Several new peaks were shown on LCMS and the desired compound was detected. The reaction mixture was filtered and diluted with water (100 mL) and extracted with ethyl acetate (100 mL×2). The combined organic phase was washed with saturated brine (200 mL), dried with anhydrous sodium sulfate, filtered and concentrated in vacuum. The residue was purified by silica gel chromatography (Petroleum ether/Ethyl acetate=1/0 to 50/1). Compound methyl 4-bromo-3-methoxy-2-methyl-benzoate (740 mg, 2.86 mmol, 89% yield) was obtained as a white solid. ¹H NMR (400 MHz, CDCl₃) δ: 7.58-7.50 (m, 1H), 7.50-7.43 (m, 1H), 3.91 (s, 3H), 3.82 (s, 3H), 2.58 (s, 3H). MS (ESI) m/z: 259.0 [M+1]⁺

### Step 3: Preparation of methyl 4-bromo-2-(bromomethyl)-3-methoxy-benzoate

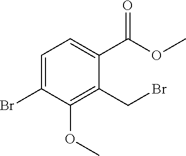

To a solution of methyl 4-bromo-3-methoxy-2-methyl-benzoate (145 mg, 0.55 mmol, 1 eq) in carbon tetrachloride (1 mL) was added n-bromosuccinimide (119 mg, 0.67 mmol, 1.2 eq) and AIBN (2 mg, 0.02 mmol, 0.03 eq). The mixture was stirred at 70° C. for 3 hr. under nitrogen atmosphere. The reaction mixture was diluted with water (50 mL) and extracted with ethyl acetate (50 mL×2). The combined organic phase was washed with saturated brine (100 mL), dried with anhydrous sodium sulfate, filtered and concentrated in vacuum. The residue was purified by silica gel chromatography (Petroleum ether/Ethyl acetate=1/0 to 10/1). The title compound was obtained as a white solid (170 mg, 0.50 mmol, 89% yield). ¹H NMR (400 MHz, CDCl₃) δ: 7.62 (dd, J=8.4, 13.6 Hz, 2H), 5.11 (s, 2H), 4.04 (s, 3H), 3.06 (s, 3H)

### Step 4: Preparation of tert-butyl 5-amino-4-(5-bromo-4-methoxy-1-oxo-isoindolin-2-yl)-5-oxo-pentanoate

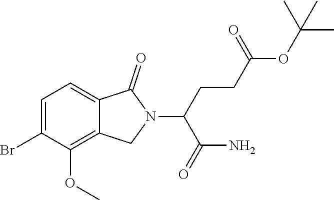 [SMILES: COc1c(Br)ccc2c1CN(C(CCC(=O)OC(C)(C)C)C(N)=O)C2=O]

To a solution of methyl 4-bromo-2-(bromomethyl)-3-methoxy-benzoate (750 mg, 2.22 mmol, 1 eq) and tert-butyl 4,5-diamino-5-oxo-pentanoate (673 mg, 3.33 mmol, 1.5 eq) in N,N-dimethylformamide (7 mL) was added N,N-diisopropylethylamine (860 mg, 6.66 mmol, 1.16 mL, 3 eq). The mixture was stirred at 110° C. for 1 h. The reaction mixture was diluted with water (100 mL) and extracted with ethyl acetate (100 mL×2). The combined organic phase was washed with saturated brine (200 mL), dried with anhydrous sodium sulfate, filtered and concentrated in vacuum. The residue was purified by silica gel chromatography (Petroleum ether/Ethyl acetate=1/0 to 1/2). Compound tert-butyl 5-amino-4-(5-bromo-4-methoxy-1-oxo-isoindolin-2-yl)-5-oxo-pentanoate (880 mg, 2.06 mmol, 92% yield) was obtained as a white solid. MS (ESI) m/z: 427.1 [M+1]⁺.

### Step 5: Preparation of benzyl 4-[2-(4-tert-butoxy-1-carbamoyl-4-oxo-butyl)-4-methoxy-1-oxo-isoindolin-5-yl]-3,6-dihydro-2H-pyridine-1-carboxylate

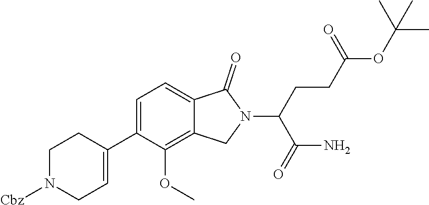 [SMILES: COc1c(C2=CCN(C(=O)OCc3ccccc3)CC2)ccc2c1CN(C(CCC(=O)OC(C)(C)C)C(N)=O)C2=O]

A mixture of tert-butyl 5-amino-4-(5-bromo-4-methoxy-1-oxo-isoindolin-2-yl)-5-oxo-pentanoate (780 mg, 1.83 mmol, 1 eq), benzyl 4-(4,4,5,5-tetramethyl-1,3,2-dioxaborolan-2-yl)-3,6-dihydro-2H-pyridine-1-carboxylate (751 mg, 2.19 mmol, 1.2 eq), ditert-butyl (cyclopentyl)phosphane;dichloropalladium;iron (118 mg, 0.18 mmol, 0.1 eq) and cesium fluoride (831 mg, 5.48 mmol, 0.2 mL, 3 eq) in dioxane (10 mL) and water (1 mL) was degassed and purged with nitrogen for 3 times, and then the mixture was stirred at 90° C. for 6 h under nitrogen atmosphere. The reaction mixture was diluted with water (200 mL). The organic layer was extracted with ethyl acetate (100 mL×2). The combined organic layer was washed with brine (200 mL). The organic layer was dried over anhydrous sodium sulfate, filtered and concentrated under vacuum to get the residue. The residue was purified by prep-HPLC (column: Phenomenex luna C18 (250*70 mm, 10 um); mobile phase: [water (0.225% FA)—ACN]; B %: 50%-75%, 17 min). Compound benzyl 4-[2-(4-tert-butoxy-1-carbamoyl-4-oxo-butyl)-4-methoxy-1-oxo-isoindolin-5-yl]-3,6-dihydro-2H-pyridine-1-carboxylate (900 mg, 1.60 mmol, 87% yield) was obtained as a white solid.). ¹H NMR (400 MHz, CDCl₃) δ: 7.56-7.49 (m, 1H), 7.46-7.32 (m, 5H), 7.30-7.23 (m, 1H), 6.54 (s, 1H), 5.96-5.78 (m, 1H), 5.69 (br s, 1H), 5.21 (m, 2H), 4.93 (dd, J=6.4, 8.4 Hz, 1H), 4.58 (d, J=17.2 Hz, 2H), 4.23-4.14 (m, 2H), 3.84 (s, 3H), 3.72 (t, J=5.2 Hz, 2H), 2.53 (br s, 2H), 2.42-2.14 (m, 4H), 1.42 (s, 9H). MS (ESI) m/z: 564.4 [M+1]⁺.

### Step 6: Preparation of tert-butyl 5-amino-4-[4-methoxy-1-oxo-5-(4-piperidyl)isoindolin-2-yl]-5-oxo-pentanoate

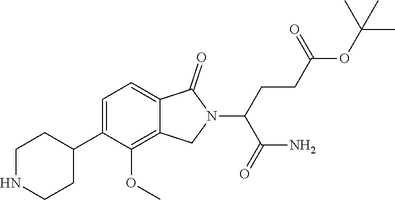 [SMILES: COc1c(C2CCNCC2)ccc2c1CN(C(CCC(=O)OC(C)(C)C)C(N)=O)C2=O]

To a solution of benzyl 4-[2-(4-tert-butoxy-1-carbamoyl-4-oxo-butyl)-4-methoxy-1-oxo-isoindolin-5-yl]-3,6-dihydro-2H-pyridine-1-carboxylate (900 mg, 1.60 mmol, 1 eq) in 2,2,2-trifluoroethanol (10 mL) and tetrahydrofuran (10 mL) was added palladium on activated carbon catalyst (200 mg, 10% purity) and palladium hydroxide on activated carbon catalyst (200 mg, 20% purity) under nitrogen atmosphere. The suspension was degassed and purged with hydrogen for three times. The mixture was stirred under hydrogen (50 Psi) at 30° C. for 12 h. The reaction mixture was filtered and concentrated under reduced pressure to give a residue. The crude product was used into the next step without further purification. Compound tert-butyl 5-amino-4-[4-methoxy-1-oxo-5-(4-piperidyl)isoindolin-2-yl]-5-oxo-pentanoate (680 mg, 1.58 mmol, 98% yield) was obtained as a white solid.). ¹H NMR (400 MHz, CDCl₃) δ: 7.47 (br d, J=7.6 Hz, 1H), 7.28 (d, J=7.6 Hz, 1H), 4.82 (br t, J=7.2 Hz, 1H), 4.62-4.41 (m, 2H), 3.96-3.96 (m, 2H), 3.85 (s, 3H), 3.65 (q, J=7.2 Hz, 2H), 2.81-2.63 (m, 2H), 2.36-2.08 (m, 4H), 1.75-1.65 (m, 2H), 1.62-1.56 (m, 1H), 1.34 (s, 9H).

### Step 7: Preparation of benzyl 4-((1s,3s)-3-(benzyloxy)cyclobutoxy)piperidine-1-carboxylate

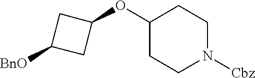 [SMILES: O=C(OCc1ccccc1)N1CCC(O[C@H]2C[C@@H](OCc3ccccc3)C2)CC1]

A mixture of cis-3-benzyloxycyclobutanol (100 g, 561.08 mmol, 1 eq) and benzyl 4-oxopiperidine-1-carboxylate (143.97 g, 617.19 mmol, 123.1 mL, 1.1 eq) in acetonitrile (2000 mL) was degassed and purged with nitrogen for 3 times, and then was added chloro(dimethyl)silane (53.09 g, 561.08 mmol, 1 eq) at 0° C. The mixture was stirred at 25° C. for 12 h under nitrogen atmosphere. LCMS showed the desired mass was detected. The reaction mixture was diluted with water (2 L). The organic layer was extracted with ethyl acetate (1 L×2). The combined organic layer was washed with brine (500 mL). The organic layer was dried over anhydrous sodium sulfate, filtered and concentrated under vacuum to get the residue. The residue was purified by silica gel chromatography (Petroleum ether/Ethyl acetate=30/1, 20/1) to get the product. The title compound was obtained as a colorless oil (89 g, 225.04 mmol, 40% yield). MS (ESI) m/z: 396.3 [M+1]⁺.

### Step 8: Preparation of benzyl 4-((1s,3s)-3-hydroxycyclobutoxy)piperidine-1-carboxylate

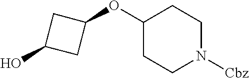 [SMILES: O=C(OCc1ccccc1)N1CCC(O[C@H]2C[C@@H](O)C2)CC1]

To a solution of benzyl 4-((1s,3s)-3-(benzyloxy)cyclobutoxy)piperidine-1-carboxylate (65 g, 164.35 mmol, 1 eq) in ethanol (300 mL) and tetrahydrofuran (300 mL) was added palladium on activated carbon catalyst (6 g, 1.44 mmol, 10% purity), palladium hydroxide on activated carbon catalyst (6 g, 8.54 mmol, 20% purity) and di-tert-butyl dicarbonate (53.80 g, 246.53 mmol, 56.6 mL, 1.5 eq) under nitrogen atmosphere. The suspension was degassed and purged with hydrogen for three times. The mixture was stirred under hydrogen (50 Psi) at 40° C. for 16 h. Thin-Layer Chromatography (Petroleum ether:Ethyl acetate=1:1) indicated the starting material was consumed completely and two new spot formed. The reaction mixture was filtered and concentrated under reduced pressure to give a residue. The crude product was purified by silica gel chromatography (Petroleum ether/Ethyl acetate=50/1, 0/1). The title product was obtained as a white (30.8 g, 113.51 mmol, 69% yield). ¹H NMR (400 MHz, CDCl₃) δ: 3.94-3.85 (m, 1H), 3.82-3.71 (m, 2H), 3.68-3.57 (m, 1H), 3.49-3.35 (m, 1H), 3.08-2.92 (m, 2H), 2.76-2.64 (m, 2H), 1.96-1.88 (m, 2H), 1.84-1.72 (m, 2H), 1.54-1.37 (m, 11H)

### Step 9: Preparation of benzyl 4-((1s,3s)-3-((tert-butylsulfonyl)oxy)cyclobutoxy)piperidine-1-carboxylate

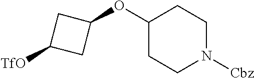 [SMILES: O=C(OCc1ccccc1)N1CCC(O[C@H]2C[C@@H](OS(=O)(=O)C(F)(F)F)C2)CC1]

To a solution of benzyl 4-((1s,3s)-3-hydroxycyclobutoxy)piperidine-1-carboxylate (4 g, 14.74 mmol, 1 eq) and triethylamine (4.47 g, 44.22 mmol, 6.16 mL, 3 eq) in dichloromethane (120 mL) was added trifluoromethanesulfonyl anhydride (4.57 g, 16.22 mmol, 2.68 mL, 1.1 eq) at 0° C. The mixture was stirred at 25° C. for 0.5 h. TLC showed the reaction was completed. The reaction was quenched with water (20 mL). The solution was extracted with dichloromethane (20 mL×2). The organic layers were combined, dried over anhydrous sodium sulfate, filtered and concentrated under reduced pressure to give a residue. The residue was purified by silica gel chromatography (2-5% ethyl acetate in petroleum ether). The title compound was obtained as a yellow solid (2.5 g, 6.20 mmol, 42% yield). ¹H NMR (400 MHz, CDCl₃) δ: 4.83 (quin, J=7.2 Hz, 1H), 3.76-3.61 (m, 3H), 3.43-3.34 (m, 1H), 2.99 (ddd, J=3.6, 9.6, 13.2 Hz, 2H), 2.88-2.74 (m, 2H), 2.48-2.21 (m, 2H), 1.74-1.65 (m, 2H), 1.45-1.36 (m, 11H).

### Step 10: Preparation of tert-butyl 4-((1r,3r)-3-(4-(2-(1-amino-5-(tert-butoxy)-1,5-dioxopentan-2-yl)-4-methoxy-1-oxoisoindolin-5-yl)piperidin-1-yl)cyclobutoxy)piperidine-1-carboxylate

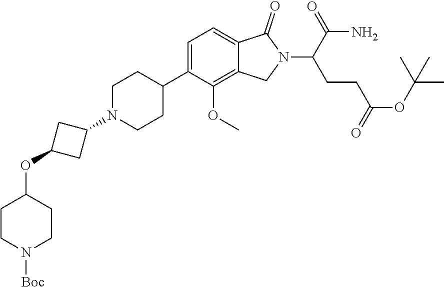 [SMILES: COc1c(C2CCN([C@H]3C[C@H](OC4CCN(C(=O)OC(C)(C)C)CC4)C3)CC2)ccc2c1CN(C(CCC(=O)OC(C)(C)C)C(N)=O)C2=O]

To a solution of tert-butyl 5-amino-4-[4-methoxy-1-oxo-5-(4-piperidyl)isoindolin-2-yl]-5-oxo-pentanoate (330 mg, 0.76 mmol, 1 eq) and benzyl 4-((1s,3s)-3-((tert-butylsulfonyl)oxy)cyclobutoxy)piperidine-1-carboxylate (339 mg, 0.84 mmol, 1.1 eq) in acetonitrile (10 mL) was added N,N-diisopropylethylamine (296 mg, 2.29 mmol, 0.3 mL, 3 eq). The mixture was stirred at 25° C. for 12 h. The reaction mixture was diluted with water (100 mL). The organic layer was extracted with ethyl acetate (100 mL×2). The combined organic layer was washed with brine (100 mL). The organic layer was dried over anhydrous sodium sulfate, filtered and concentrated under vacuum to get the residue. The residue was purified by prep-HPLC (column: Phenomenex luna C18 (250*70 mm, 10 um); mobile phase: [water (0.225% FA)—ACN]; B %: 10%-40%, 20 min) to obtain the title compound as a yellow oil (290 mg, 0.42 mmol, 55% yield). MS (ESI) m/z: 685.3 [M+1]⁺.

### Step 11: Preparation of 3-(4-methoxy-1-oxo-5-(1-((1r,3r)-3-(piperidin-4-yloxy)cyclobutyl)piperidin-4-yl)isoindolin-2-yl)piperidine-2,6-dione

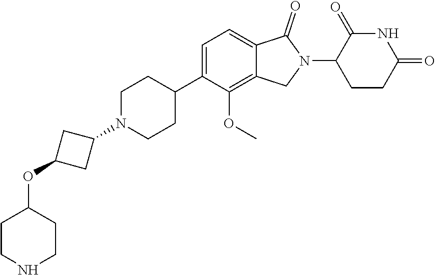 [SMILES: COc1c(C2CCN([C@H]3C[C@H](OC4CCNCC4)C3)CC2)ccc2c1CN(C1CCC(=O)NC1=O)C2=O]

A mixture of tert-butyl 4-((1r,3r)-3-(4-(2-(1-amino-5-(tert-butoxy)-1,5-dioxopentan-2-yl)-4-methoxy-1-oxoisoindolin-5-yl)piperidin-1-yl)cyclobutoxy)piperidine-1-carboxylate (290 mg, 0.42 mmol, 1 eq) and [(1R,4S)-7,7-dimethyl-2-oxo-norbornan-1-yl]methanesulfonic acid (245 mg, 1.06 mmol, 2.5 eq) in acetonitrile (10 mL) was stirred at 80° C. for 12 h. The reaction mixture was basified by N,N-diisopropylethylamine and then concentrated under reduced pressure to give a residue. The crude product was used into the next step without further purification. The title compound was obtained as a colorless gum (260 mg, 0.41 mmol, 98% yield, trifluoroacetate). MS (ESI) m/z: 529.3 [M+18]⁺.

Step 12: Preparation of 2-({6-[(5-chloro-2-{4-[(1r,3r)-3-{4-[2-(2,6-dioxopiperidin-3-yl)-1,3-dioxo-2,3-dihydro-1H-isoindol-5-yl]piperidin-1-yl}cyclobutoxy]piperidin-1-yl}pyrimidin-4-yl)amino]-1-ethyl-2-oxo-1,2-dihydroquinolin-3-yl}oxy)-N-methylacetamide

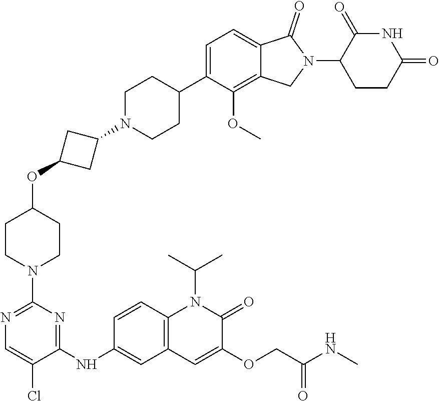 [SMILES: CNC(=O)COc1cc2cc(Nc3nc(N4CCC(O[C@H]5C[C@H](N6CCC(c7ccc8c(c7OC)CN(C7CCC(=O)NC7=O)C8=O)CC6)C5)CC4)ncc3Cl)ccc2n(C(C)C)c1=O]

To a solution of 3-(4-methoxy-1-oxo-5-(1-((1r,3r)-3-(piperidin-4-yloxy)cyclobutyl)piperidin-4-yl)isoindolin-2-yl)piperidine-2,6-dione (260 mg, 0.41 mmol, 1 eq, trifluoroacetate) and 2-[[6-[(2,5-dichloropyrimidin-4-yl)amino]-1-isopropyl-2-oxo-3-quinolyl]oxy]-N-methyl-acetamide (181 mg, 0.41 mmol, 1 eq) in dimethyl sulfoxide (3 mL) was added N,N-diisopropylethylamine (161 mg, 1.25 mmol, 0.2 mL, 3 eq). The mixture was stirred at 120° C. for 1 h. The reaction mixture was filtered and concentrated under reduced pressure to give a residue. The residue was purified by prep-HPLC (column: Phenomenex Synergi C18 150*25 mm*10 um; mobile phase: [water (0.1% TFA)—ACN]; B %: 18%-48%, 10 min). The title compound was obtained as a white solid (47.8 mg, 0.05 mmol, 12% yield, 98% purity). ¹H NMR (400 MHz, DMSO-d₆) δ: 10.9 (s, 1H), 8.83 (s, 1H), 8.05 (s, 1H), 8.01-7.92 (m, 2H), 7.69 (s, 2H), 7.40 (s, 2H), 7.03 (s, 1H), 5.59-5.19 (m, 1H), 5.10 (dd, J=5.2, 13.2 Hz, 1H), 4.70-4.60 (m, 1H), 4.55 (s, 2H), 4.50-4.42 (m, 1H), 4.24-4.11 (m, 2H), 3.91 (s, 3H), 3.54 (br s, 1H), 3.30 (s, 3H), 3.27-3.19 (m, 2H), 3.05-2.86 (m, 4H), 2.68 (d, J=4.4 Hz, 3H), 2.65-2.59 (m, 2H), 2.16 (br s, 2H), 2.04-19.6 (m, 3H), 1.88-1.76 (m, 4H), 1.72-1.66 (m, 3H), 1.58 (d, J=7.2 Hz, 6H), 1.43-1.33 (m, 2H). MS (ESI) m/z: 910.2 [M+1]⁺.

Example 12: Preparation of 2-({6-[(5-chloro-2-{4-[(1r,3r)-3-{4-[2-(2,6-dioxopiperidin-3-yl)-6-fluoro-1-oxo-2,3-dihydro-1H-isoindol-5-yl]piperidin-1-yl}cyclobutoxy]piperidin-1-yl}pyrimidin-4-yl)amino]-2-oxo-1-(propan-2-yl)-1,2-dihydroquinolin-3-yl}oxy)-N-methylacetamide (Compound 79)

### Step 1: Preparation of methyl 4-bromo-5-fluoro-2-methyl-benzoate

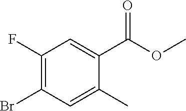

To a solution of 4-bromo-5-fluoro-2-methyl-benzoic acid (10.50 g, 45.06 mmol, 1.00 eq) in dimethyl formamide (110 mL) were added potassium carbonate (15.57 g, 112.64 mmol, 2.50 eq) and iodomethane (19.19 g, 135.17 mmol, 8.4 mL, 3.00 eq) at 20° C. and the mixture was stirred at 20° C. for 2 h. Thin layer chromatography (dichloromethane:methanol=10:1) showed the reaction was completed. The mixture was filtered, and the filtrate was diluted with water (600 mL) and extracted with ethyl acetate (50 mL). The organic layer was washed with water (600 mL×2), brine (600 mL×2), dried over sodium sulfate and then concentrated under reduced pressure to give methyl 4-bromo-5-fluoro-2-methyl-benzoate (11.00 g, 44.52 mmol, 99% yield) as a yellow oil. ¹H NMR (400 MHz, CDCl₃) δ: 7.68 (d, J=9.2 Hz, 1H), 7.46 (d, J=6.4 Hz, 1H), 3.91 (s, 3H), 2.56 (s, 3H).

### Step 2: Preparation of methyl 4-bromo-2-(bromomethyl)-5-fluoro-benzoate

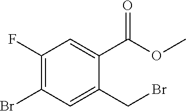

To a solution of methyl 4-bromo-5-fluoro-2-methyl-benzoate (11 g, 44.52 mmol, 1.00 eq) in dichloroethane (150 mL) were added N-Bromosuccinimide (8.72 g, 48.98 mmol, 1.10 eq) and 2, 2-azobisisobutyronitrile (731 mg, 4.45 mmol, 0.10 eq) at 20° C. and the mixture was warmed to 80° C. The mixture was stirred at 80° C. for 6 h. Thin layer chromatography (petroleum ether:ethyl acetate=3:1) showed the reaction was completed. The mixture was filtered, and the filtrate was diluted with saturated sodium thiosulfate (500 mL) and extracted with dichloromethane (300 mL). The organic layer was washed with water (500 mL×2), brine (500 mL×2), dried over sodium sulfate and then concentrated under reduced pressure to give a residue. The residue was purified by column chromatography on silica gel (petroleum ether:ethyl acetate=30:1 to 20:1) to give methyl 4-bromo-2-(bromomethyl)-5-fluoro-benzoate (13.00 g, 39.88 mmol, 90% yield) as a colorless oil. ¹H NMR (400 MHz, CDCl₃) δ: 7.75-7.67 (m, 2H), 4.89 (s, 2H), 3.95 (s, 3H).

### Step 3: Preparation of tert-butyl 5-amino-4-(5-bromo-6-fluoro-1-oxo-isoindolin-2-yl)-5-oxo-pentanoate

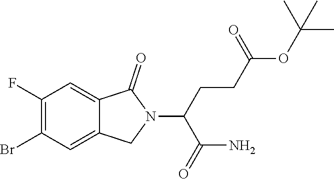 [SMILES: CC(C)(C)OC(=O)CCC(C(N)=O)N1Cc2cc(Br)c(F)cc2C1=O]

To a solution of methyl 4-bromo-2-(bromomethyl)-5-fluoro-benzoate (2.00 g, 6.14 mmol, 1.00 eq) in dimethyl formamide (20 mL) were added diisopropylethylamine (3.17 g, 24.54 mmol, 4.3 mL, 4.00 eq) and tert-butyl 4,5-diamino-5-oxo-pentanoate (1.24 g, 6.14 mmol, 1.00 eq) at 80° C. and the mixture was stirred at 80° C. for 12 h. Thin layer chromatography (dichloromethane:methanol=20:1) showed the reaction was completed. The mixture was diluted with water (100 mL) and extracted with ethyl acetate (100 mL). The organic layer was washed with water (100 mL×2 mL), brine (100 mL), dried over sodium sulfate and then concentrated under reduced pressure to give a light-yellow solid. The solid was triturated with petroleum ether:ethyl acetate (80 mL, 3:1) to give tert-butyl 5-amino-4-(5-bromo-6-fluoro-1-oxo-isoindolin-2-yl)-5-oxo-pentanoate (4.50 g, 10.84 mmol, 88% yield) as a white solid. δ: 8.02 (d, J=6.0 Hz, 1H), 7.72-7.54 (m, 2H), 7.24 (s, 1H), 4.79-4.67 (m, 1H), 4.65-4.55 (m, 1H), 4.52-4.35 (m, 1H), 2.23-2.09 (m, 3H), 2.05-1.90 (m, 1H), 1.33 (s, 9H).

### Step 4: Preparation of benzyl 4-[2-(4-tert-butoxy-1-carbamoyl-4-oxo-butyl)-6-fluoro-1-oxo-isoindolin-5-yl]-3,6-dihydro-2H-pyridine-1-carboxylate

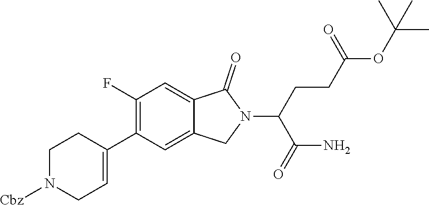 [SMILES: CC(C)(C)OC(=O)CCC(C(N)=O)N1Cc2cc(C3=CCN(C(=O)OCc4ccccc4)CC3)c(F)cc2C1=O]

The title compound was prepared analogously to Example 11, Step 5. The crude product was purified by column chromatography on silica gel (petroleum ether:ethyl acetate=5:1 to 0:1) to give benzyl 4-[2-(4-tert-butoxy-1-carbamoyl-4-oxo-butyl)-6-fluoro-1-oxo-isoindolin-5-yl]-3,6-dihydro-2H-pyridine-1-carboxylate (2.20 g, 3.91 mmol, 81% yield, 98% purity) as a light brown solid. ¹H NMR (400 MHz, CDCl₃) δ: 7.47 (d, J=9.6 Hz, 1H), 7.44-7.28 (m, 6H), 6.40 (s, 1H), 5.97 (d, J=9.6 Hz, 1H), 5.54 (s, 1H), 5.19 (s, 2H), 4.90 (dd, J=6.4, 8.4 Hz, 1H), 4.58-4.48 (m, 1H), 4.45-4.36 (m, 1H), 4.18 (d, J=2.4 Hz, 2H), 3.72 (t, J=5.2 Hz, 2H), 2.53 (br s, 2H), 2.43-2.09 (m, 4H), 1.42 (s, 9H). MS (ESI) m/z: 552.2 [M+1]⁺.

### Step 5: Preparation of tert-butyl 5-amino-4-[6-fluoro-1-oxo-5-(4-piperidyl)isoindolin-2-yl]-5-oxo-pentanoate

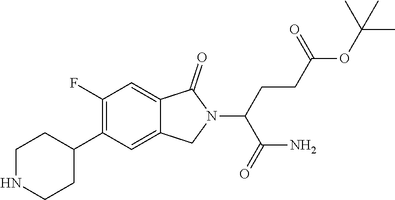 [SMILES: CC(C)(C)OC(=O)CCC(C(N)=O)N1Cc2cc(C3CCNCC3)c(F)cc2C1=O]

The title compound was prepared analogously to Example 11, Step 6. The crude product (720 mg, 1.72 mmol, 95% yield) was used in the next step without further purification. ¹H NMR (400 MHz, DMSO-d₆) δ: 7.66-7.51 (m, 2H), 7.42 (d, J=9.2 Hz, 1H), 7.20 (s, 1H), 4.76-4.67 (m, 1H), 4.62-4.51 (m, 1H), 4.47-4.36 (m, 1H), 3.12-2.93 (m, 3H), 2.66 (t, J=11.2 Hz, 2H), 2.16 (s, 3H), 2.00-1.94 (m, 1H), 1.78-1.55 (m, 4H), 1.32 (s, 9H). MS (ESI) m/z: 420.2 [M+1]⁺.

### Step 6: Preparation of tert-butyl 4-((1r,3r)-3-(4-(2-(1-amino-5-(tert-butoxy)-1,5-dioxopentan-2-yl)-6-fluoro-1-oxoisoindolin-5-yl)piperidin-1-yl)cyclobutoxy)piperidine-1-carboxylate

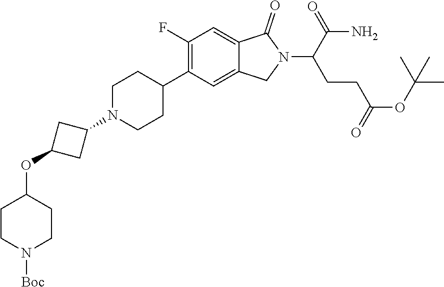 [SMILES: CC(C)(C)OC(=O)CCC(C(N)=O)N1Cc2cc(C3CCN([C@H]4C[C@H](OC5CCN(C(=O)OC(C)(C)C)CC5)C4)CC3)c(F)cc2C1=O]

The title compound was prepared analogously to Example 11, step 10. The crude product was purified by column chromatography on silica gel (petroleum ether:ethyl acetate=1:1 to dichloromethane:methanol=20:1) to afford the title product as a light yellow oil (600 mg, 0.89 mmol, 53% yield). MS (ESI) m/z: 673.3 [M+1]⁺.

### Step 7: Preparation of 3-(6-fluoro-1-oxo-5-(1-((1r,3r)-3-(piperidin-4-yloxy)cyclobutyl)piperidin-4-yl)isoindolin-2-yl)piperidine-2,6-dione

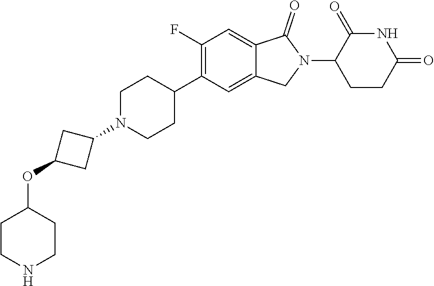 [SMILES: O=C1CCC(N2Cc3cc(C4CCN([C@H]5C[C@H](OC6CCNCC6)C5)CC4)c(F)cc3C2=O)C(=O)N1]

The title compound was prepared analogously to Example 11, Step 11. The crude product was purified by preparative high performance liquid chromatography (column: 3_Phenomenex Luna C18 75*30 mm*3 um; mobile phase: [water(0.1% TFA)-ACN];B %: 2%-32%, 7 min) to give the title product as a white solid (200 mg, 0.33 mmol, 56% yield, trifluoroacetate). MS (ESI) m/z: 613.2 [M+1]⁺.

Step 8: Preparation of 2-({6-[(5-chloro-2-{4-[(1r,3r)-3-{4-[2-(2,6-dioxopiperidin-3-yl)-6-fluoro-1-oxo-2,3-dihydro-1H-isoindol-5-yl]piperidin-1-yl}cyclobutoxy]piperidin-1-yl}pyrimidin-4-yl)amino]-2-oxo-1-(propan-2-yl)-1,2-dihydroquinolin-3-yl}oxy)-N-methylacetamide

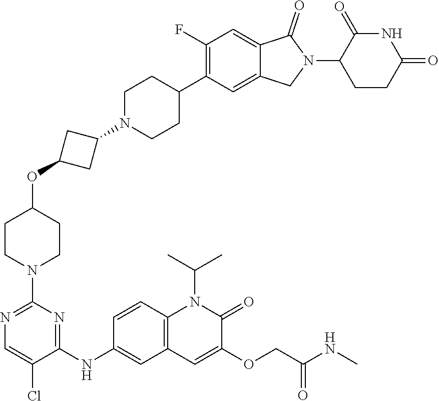 [SMILES: CNC(=O)COc1cc2cc(Nc3nc(N4CCC(O[C@H]5C[C@H](N6CCC(c7cc8c(cc7F)C(=O)N(C7CCC(=O)NC7=O)C8)CC6)C5)CC4)ncc3Cl)ccc2n(C(C)C)c1=O]

The title compound was prepared analogously to Example 11, Step 12. The crude product was purified by preparative high performance liquid chromatography (column: Phenomenex Synergi C18 150*25 mm*10 um; mobile phase: [water(0.225% FA)-ACN];B %: 11%-41%, 10 min) to give the title product as a white solid (77.4 mg, 25% yield). ¹H NMR (400 MHz, DMSO-d₆) δ: 11.00 (s, 1H), 8.83 (s, 1H), 8.16 (s, 1H), 8.05 (s, 1H), 7.99-7.90 (m, 2H), 7.69 (s, 2H), 7.62 (d, J=6.0 Hz, 1H), 7.50-7.43 (m, 1H), 7.03 (s, 1H), 5.64-5.18 (m, 1H), 5.16-5.05 (m, 1H), 4.61-4.50 (m, 2H), 4.47-4.39 (m, 1H), 4.33-4.26 (m, 1H), 4.22-4.16 (m, 1H), 4.15-4.07 (m, 2H), 3.57-3.50 (m, 1H), 3.24 (t, J=10.4 Hz, 2H), 3.02 (d, J=10.4 Hz, 2H), 2.94-2.86 (m, 2H), 2.68 (d, J=4.8 Hz, 3H), 2.64-2.58 (m, 2H), 2.42-2.36 (m, 2H), 2.21-2.14 (m, 2H), 2.03-1.98 (m, 2H), 1.86-1.79 (m, 4H), 1.77-1.69 (m, 4H), 1.57 (d, J=6.8 Hz, 6H), 1.43-1.34 (m, 2H).

Example 13: Preparation of Compound A, i.e.: 2-({6-[(5-chloro-2-{4-[(1r,3r)-3-{4-[2-(2,6-dioxopiperidin-3-yl)-4-fluoro-1-oxo-2,3-dihydro-1H-isoindol-5-yl]piperidin-1-yl}cyclobutoxy]piperidin-1-yl}pyrimidin-4-yl)amino]-2-oxo-1-(propan-2-yl)-1,2-dihydroquinolin-3-yl}oxy)-N-methylacetamide (Compound 81)

### Step 1: Preparation of 4-bromo-3-fluoro-2-methyl-benzoic acid

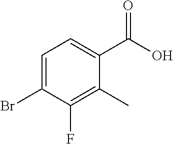

To a solution of 4-bromo-3-fluoro-benzoic acid (20.00 g, 91.32 mmol, 1.00 eq) in tetrahydrofuran (200 mL) was added lithium diisopropylamide (2 M, 96.0 mL, 2.10 eq) at −70° C. and the mixture was stirred at −70° C. for 1 h. Then iodomethane (38.89 g, 273.96 mmol, 17.1 mL, 3.00 eq) was added at −70° C. Then the mixture was warmed to 20° C. and the mixture was stirred at 20° C. for 12 h. The mixture was quenched with saturated ammonium chloride solution (400 mL) and extracted with ethyl acetate (400 mL). The organic layer was dried over sodium sulfate and then concentrated under reduced pressure to give 4-bromo-3-fluoro-2-methyl-benzoic acid as a yellow solid (16.00 g, 68.66 mmol, 75% yield). ¹H NMR (400 MHz, DMSO-d₆) δ 7.55-7.47 (m, 1H), 7.46-7.36 (m, 1H), 2.42 (d, J=2.0 Hz, 3H).

### Step 2: Preparation of methyl 4-bromo-3-fluoro-2-methyl-benzoate

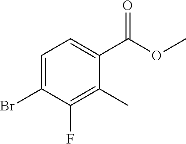

To a solution of 4-bromo-3-fluoro-2-methyl-benzoic acid (14.00 g, 60.08 mmol, 1.00 eq) in methanol (100 mL) was added thionyl chloride (42.88 g, 360.46 mmol, 26.1 mL, 6.00 eq) at 20° C. and the mixture was stirred at 20° C. for 1 h. The mixture was concentrated under reduced pressure to give a residue. The residue was quenched with saturated sodium bicarbonate solution (1000 mL) and extracted with ethyl acetate (500 mL). The organic layer was dried over sodium sulfate and then concentrated under reduced pressure to give a residue. The residue was purified by column chromatography on silica gel (petroleum ether:ethyl acetate=10:1) to give methyl 4-bromo-3-fluoro-2-methyl-benzoate (6.00 g, 24.09 mmol, 40% yield) as a colorless oil. ¹H NMR (400 MHz, CDCl₃) δ 7.58-7.52 (m, 1H), 7.45-7.39 (m, 1H), 3.90 (s, 3H), 2.53 (d, J=2.6 Hz, 3H).

### Step 3: Preparation of methyl 4-bromo-2-(bromomethyl)-3-fluoro-benzoate

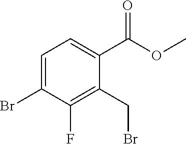

To a solution of methyl 4-bromo-3-fluoro-2-methyl-benzoate (6.20 g, 25.10 mmol, 1.00 eq) in dichloroethane (70 mL) were added N-Bromosuccinimide (4.91 g, 27.60 mmol, 1.10 eq) and 2, 2-azobisisobutyronitrile (412.09 mg, 2.51 mmol, 0.10 eq) at 20° C. and the mixture was warmed to 80° C. The mixture was stirred at 80° C. for 6 h. The mixture was filtered, and the filtrate was diluted with saturated sodium thiosulfate solution (100 mL) and extracted with dichloromethane (50 mL). The organic layer was washed with water (100 mL×2), brine (100 mL), dried over sodium sulfate and then concentrated under reduced pressure to give a residue. The residue was purified by column chromatography on silica gel (petroleum ether:ethyl acetate=30:1 to 20:1) to give methyl 4-bromo-2-(bromomethyl)-3-fluoro-benzoate (7.00 g, 21.48 mmol, 86% yield) as a colorless oil. ¹H NMR (400 MHz, CDCl₃) δ 7.71-7.62 (m, 1H), 7.62-7.51 (m, 1H), 5.00 (s, 2H), 3.96 (s, 3H).

Steps 4-9: Preparation of 2-({6-[(5-chloro-2-{4-[(1r,3r)-3-{4-[2-(2,6-dioxopiperidin-3-yl)-4-fluoro-1-oxo-2,3-dihydro-1H-isoindol-5-yl]piperidin-1-yl}cyclobutoxy]piperidin-1-yl}pyrimidin-4-yl)amino]-2-oxo-1-(propan-2-yl)-1,2-dihydroquinolin-3-yl}oxy)-N-methylacetamide

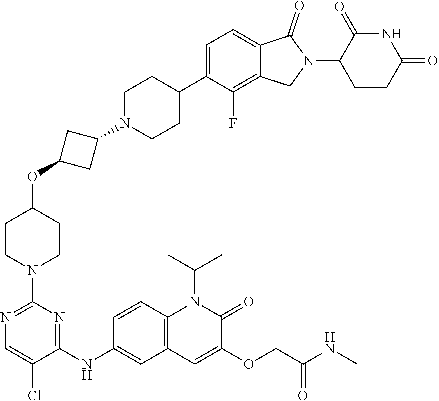 [SMILES: CNC(=O)COc1cc2cc(Nc3nc(N4CCC(O[C@H]5C[C@H](N6CCC(c7ccc8c(c7F)CN(C7CCC(=O)NC7=O)C8=O)CC6)C5)CC4)ncc3Cl)ccc2n(C(C)C)c1=O]

Compound A was prepared analogously to Example 12 following Steps 3-8 with the material made in step 3 of this Example. The crude product was purified by preparative high performance liquid chromatography (column: Phenomenex Synergi C18 150*25 mm*10 um; mobile phase: [water(0.225% FA)-ACN];B %: 11%-41%, 10 min) to afford the title product as a white solid (83.5 mg, 22% yield, formate). ¹H NMR (400 MHz, DMSO-d₆) δ: 11.00 (s, 1H), 8.83 (s, 1H), 8.18 (s, 1H), 8.04 (s, 1H), 7.99-7.91 (m, 2H), 7.69 (s, 2H), 7.61-7.47 (m, 2H), 7.03 (s, 1H), 5.58-5.15 (m, 1H), 5.11 (dd, J=5.2, 13.2 Hz, 1H), 4.59-4.50 (m, 3H), 4.37 (d, J=17.4 Hz, 1H), 4.24-4.06 (m, 3H), 3.56-3.51 (m, 1H), 3.24 (t, J=10.4 Hz, 2H), 3.01 (d, J=10.0 Hz, 2H), 2.97-2.82 (m, 3H), 2.68 (d, J=4.8 Hz, 3H), 2.60 (d, J=16.0 Hz, 1H), 2.46-2.39 (m, 1H), 2.21-2.13 (m, 2H), 2.05-1.96 (m, 3H), 1.87-1.79 (m, 4H), 1.78-1.69 (m, 4H), 1.57 (d, J=6.8 Hz, 6H), 1.43-1.34 (m, 2H). MS (ESI) m/z: 748.2 [M+1]⁺.

The contents of all references, patents, pending patent applications and published patents, cited throughout this application are hereby expressly incorporated by reference.

Those skilled in the art will recognize, or be able to ascertain using no more than routine experimentation, many equivalents to the specific embodiments of the disclosure described herein. Such equivalents are intended to be encompassed by the following claims. It is understood that the detailed examples and embodiments described herein are given by way of example for illustrative purposes only, and are in no way considered to be limiting to the disclosure. Various modifications or changes in light thereof will be suggested to persons skilled in the art and are included within the spirit and purview of this application and are considered within the scope of the appended claims. For example, the relative quantities of the ingredients may be varied to optimize the desired effects, additional ingredients may be added, and/or similar ingredients may be substituted for one or more of the ingredients described. Additional advantageous features and functionalities associated with the systems, methods, and processes of the present disclosure will be apparent from the appended claims. Moreover, those skilled in the art will recognize, or be able to ascertain using no more than routine experimentation, many equivalents to the specific embodiments of the disclosure described herein. Such equivalents are intended to be encompassed by the following claims.

1. A method of treating or ameliorating diffuse large B-cell lymphoma (DLBCL), angioimmunoblastic T-cell lymphoma, transformed follicular lymphoma, high grade B-cell lymphoma, non-Hodgkin lymphoma not otherwise specified, or solid tumors in a subject in need thereof, comprising administering to the subject an effective amount of a compound, wherein the compound is: 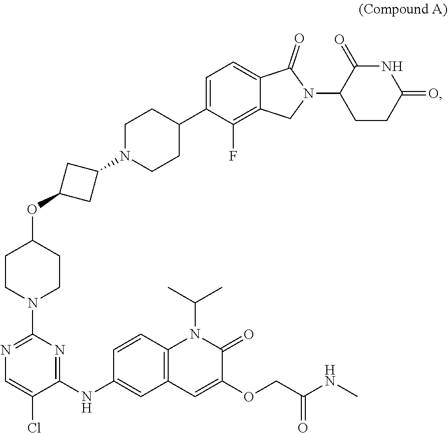 [SMILES: CNC(=O)COc1cc2cc(Nc3nc(N4CCC(O[C@H]5C[C@H](N6CCC(c7ccc8c(c7F)CN(C7CCC(=O)NC7=O)C8=O)CC6)C5)CC4)ncc3Cl)ccc2n(C(C)C)c1=O]
or a pharmaceutically acceptable salt thereof.

2. (canceled)

3. A method of treating or ameliorating diffuse large B-cell lymphoma (DLBCL) in a subject in need thereof, comprising administering to the subject an effective amount of Compound A: 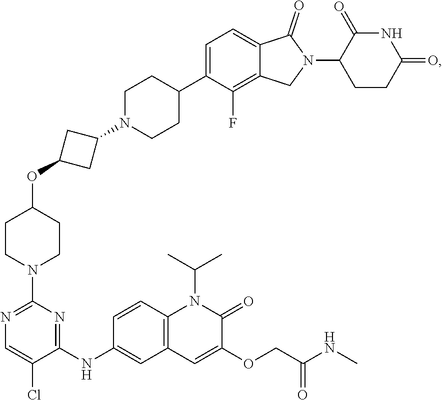 [SMILES: CNC(=O)COc1cc2cc(Nc3nc(N4CCC(O[C@H]5C[C@H](N6CCC(c7ccc8c(c7F)CN(C7CCC(=O)NC7=O)C8=O)CC6)C5)CC4)ncc3Cl)ccc2n(C(C)C)c1=O]
or a pharmaceutically acceptable salt thereof.

4. (canceled)

5. A method of treating or ameliorating angioimmunoblastic T-cell lymphoma in a subject in need thereof, comprising administering to the subject an effective amount of Compound A: 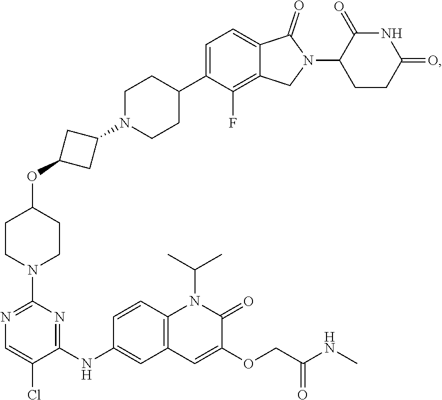 [SMILES: CNC(=O)COc1cc2cc(Nc3nc(N4CCC(O[C@H]5C[C@H](N6CCC(c7ccc8c(c7F)CN(C7CCC(=O)NC7=O)C8=O)CC6)C5)CC4)ncc3Cl)ccc2n(C(C)C)c1=O]
or a pharmaceutically acceptable salt thereof.

6. (canceled)

7. A method of treating or ameliorating transformed follicular lymphoma in a subject in need thereof, comprising administering to the subject an effective amount of Compound A: 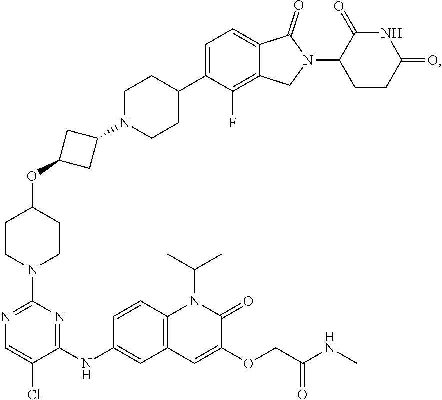 [SMILES: CNC(=O)COc1cc2cc(Nc3nc(N4CCC(O[C@H]5C[C@H](N6CCC(c7ccc8c(c7F)CN(C7CCC(=O)NC7=O)C8=O)CC6)C5)CC4)ncc3Cl)ccc2n(C(C)C)c1=O]
or a pharmaceutically acceptable salt thereof.

8. (canceled)

9. The method of claim 1, wherein the method is for treating or ameliorating diffuse large B-cell lymphoma (DLBCL), angioimmunoblastic T-cell lymphoma, transformed follicular lymphoma, high grade B-cell lymphoma, or non-Hodgkin lymphoma not otherwise specified.

10. The method of claim 1, wherein the method is for treating or ameliorating diffuse large B-cell lymphoma (DLBCL), angioimmunoblastic T-cell lymphoma, transformed follicular lymphoma, or high grade B-cell lymphoma.

11. The method of claim 1, wherein the method is for treating or ameliorating diffuse large B-cell lymphoma (DLBCL).

12. The method of claim 1, wherein the method is for treating or ameliorating diffuse large B-cell lymphoma (DLBCL), wherein the diffuse large B-cell lymphoma (DLBCL) is selected from germinal center B-cell (GCB) DLBCL and activated B-cell (ABC) DLBCL.

13. The method of claim 1, wherein the method is for treating or ameliorating angioimmunoblastic T-cell lymphoma.

14. The method of claim 1, wherein the method is for treating or ameliorating transformed follicular lymphoma.

15. The method of claim 1, wherein the method is for treating or ameliorating high grade B-cell lymphoma.

16. The method of claim 1, wherein the method is for treating or ameliorating non-Hodgkin lymphoma not otherwise specified.

17. The method of claim 1, wherein the method is for treating or ameliorating solid tumors, wherein the solid tumors are selected from breast cancer, lung cancer, ovarian cancer, neuroblastoma, and glioblastoma.

18. (canceled)

19. (canceled)

20. A method of treating or ameliorating high grade B-cell lymphoma in a subject in need thereof, comprising administering to the subject an effective amount of Compound A: 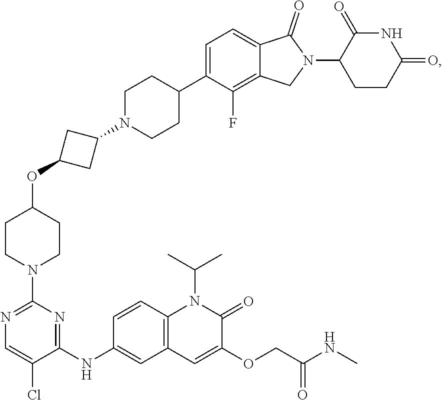 [SMILES: CNC(=O)COc1cc2cc(Nc3nc(N4CCC(O[C@H]5C[C@H](N6CCC(c7ccc8c(c7F)CN(C7CCC(=O)NC7=O)C8=O)CC6)C5)CC4)ncc3Cl)ccc2n(C(C)C)c1=O]
or a pharmaceutically acceptable salt thereof.

21. A method of treating or ameliorating diffuse large B-cell lymphoma (DLBCL), angioimmunoblastic T-cell lymphoma, transformed follicular lymphoma, high grade B-cell lymphoma, non-Hodgkin lymphoma not otherwise specified, or solid tumors in a subject in need thereof, comprising administering to the subject an effective amount of a compound, wherein the compound is: 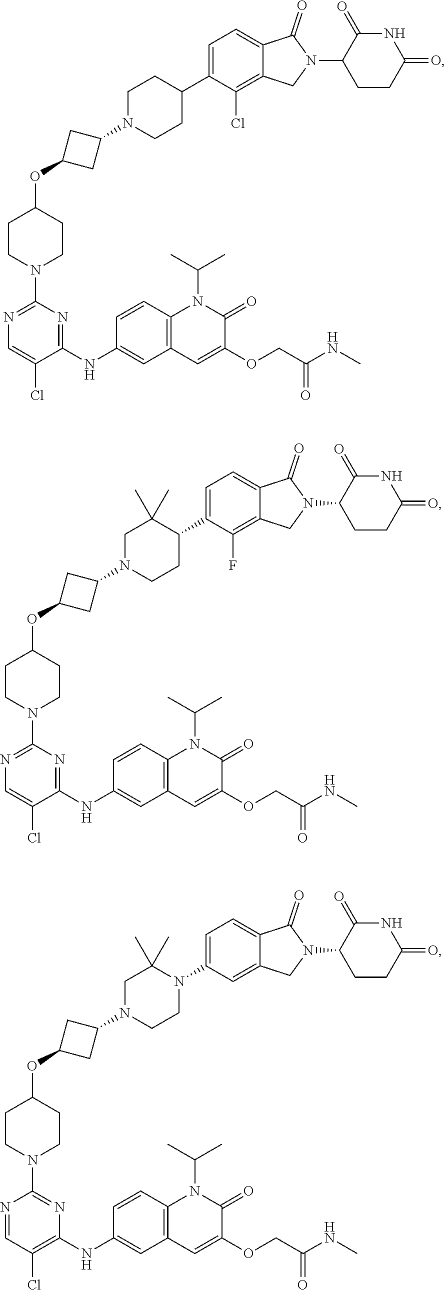 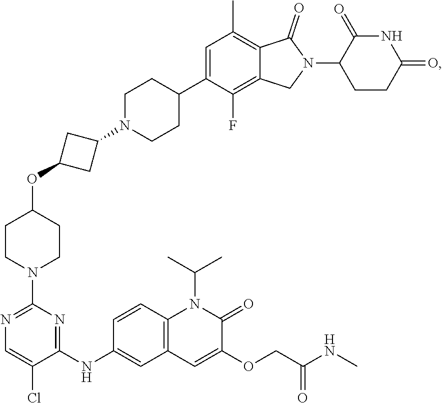 [SMILES: CNC(=O)COc1cc2cc(Nc3nc(N4CCC(O[C@H]5C[C@H](N6CCC(c7cc(C)c8c(c7F)CN(C7CCC(=O)NC7=O)C8=O)CC6)C5)CC4)ncc3Cl)ccc2n(C(C)C)c1=O]
or a pharmaceutically acceptable salt thereof.
